(19) United States
(12) Patent Application Publication
(10) Pub. No.: US 2011/0312996 A1
(43) Pub. Date: Dec. 22, 2011

(54) NOVEL INHIBITORS OF HEPATITIS C VIRUS REPLICATION

(75) Inventors: Brad Buckman, Oakland, CA (US); John B. Nicholas, Redwood City, CA (US); Vladimir Serebryany, Burlingame, CA (US); Scott D. Seiwert, Pacifica, CA (US)

(73) Assignee: InterMune, Inc., Brisbane, CA (US)

(21) Appl. No.: 13/108,211

(22) Filed: May 16, 2011

Related U.S. Application Data
(60) Provisional application No. 61/345,222, filed on May 17, 2010.
(60) Provisional application No. 61/345,553, filed on May 17, 2010.
(60) Provisional application No. 61/354,671, filed on Jun. 14, 2010.
(60) Provisional application No. 61/361,328, filed on Jul. 2, 2010.
(60) Provisional application No. 61/382,872, filed on Sep. 14, 2010.
(60) Provisional application No. 61/405,138, filed on Oct. 20, 2010.
(60) Provisional application No. 61/425,718, filed on Dec. 21, 2010.
(60) Provisional application No. 61/454,438, filed on Mar. 18, 2011.

(51) Int. Cl. A61K 31/4709; C07D 401/12; C07D 233/28; C07D 403/12; C07D 403/14; C07D 417/14; C07D 409/14; C07D 413/14; C07D 405/14; C07D 401/14; C07D 487/04; C07D 471/04; A61K 31/4184; A61K 31/4178; A61K 31/428; A61K 31/423; A61P 31/14; A61P 1/16; C07D 403/04

(52) U.S. Cl. 514/314; 548/314.7; 546/279.1; 548/334.1; 544/372; 548/305.7; 548/455; 548/156; 548/311.4; 548/219; 548/313.1; 548/518; 546/167; 544/284; 544/328; 544/280; 548/312.1; 548/305.1; 546/112; 540/593; 548/217; 548/159; 544/119; 544/296; 514/394; 514/397; 514/367; 514/375

(57) ABSTRACT

The embodiments provide compounds of the general Formulae VI, VII, VIII, IX, X, XI, XII, XIII, XIV, and XV as well as compositions, including pharmaceutical compositions, comprising a subject compound. The embodiments further provide treatment methods, including methods of treating a hepatitis C virus infection and methods of treating liver fibrosis, the methods generally involving administering to an individual in need thereof an effective amount of a subject compound or composition.

### RELATED APPLICATIONS

This application claims the benefit of U.S. Provisional Application Nos. 61/345,222, filed May 17, 2010; 61/345,553, filed May 17, 2010; 61/354,671, filed Jun. 14, 2010; 61/361,328, filed Jul. 2, 2010; 61/382,872, filed Sep. 14, 2010; 61/405,138, filed Oct. 20, 2010; 61/425,718, filed Dec. 21, 2010; and 61/454,438, filed Mar. 18, 2011; all of which are incorporated herein by reference in their entirety.

### BACKGROUND OF THE INVENTION

1. Field of the Invention

The embodiments described herein relate to compounds, processes for their synthesis, compositions and methods for the therapeutic use of the compounds, such as for treating hepatitis C virus (HCV) infection.

2. Description of the Related Art

Hepatitis C virus (HCV) infection is the most common chronic blood borne infection in the United States. Although the numbers of new infections have declined, the burden of chronic infection is substantial, with Centers for Disease Control estimates of 3.9 million (1.8%) infected persons in the United States. Chronic liver disease is the tenth leading cause of death among adults in the United States, and accounts for approximately 25,000 deaths annually, or approximately 1% of all deaths. Studies indicate that 40% of chronic liver disease is HCV-related, resulting in an estimated 8,000-10,000 deaths each year. HCV-associated end-stage liver disease is the most frequent indication for liver transplantation among adults.

Antiviral therapy of chronic hepatitis C has evolved rapidly over the last decade, with significant improvements seen in the efficacy of treatment. Nevertheless, even with combination therapy using pegylated IFN-α plus ribavirin, 40% to 50% of patients fail therapy; they are nonresponders or relapsers. These patients currently have no effective therapeutic alternative. In particular, patients who have advanced fibrosis or cirrhosis on liver biopsy are at significant risk of developing complications of advanced liver disease, including ascites, jaundice, variceal bleeding, encephalopathy, and progressive liver failure, as well as a markedly increased risk of hepatocellular carcinoma.

The high prevalence of chronic HCV infection has important public health implications for the future burden of chronic liver disease in the United States. Data derived from the National Health and Nutrition Examination Survey (NHANES III) indicate that a large increase in the rate of new HCV infections occurred from the late 1960s to the early 1980s, particularly among persons between 20 to 40 years of age. It is estimated that the number of persons with long-standing HCV infection of 20 years or longer could more than quadruple from 1990 to 2015, from 750,000 to over 3 million. The proportional increase in persons infected for 30 or 40 years would be even greater. Since the risk of HCV-related chronic liver disease is related to the duration of infection, with the risk of cirrhosis progressively increasing for persons infected for longer than 20 years, this will result in a substantial increase in cirrhosis-related morbidity and mortality among patients infected between the years of 1965-1985.

HCV is an enveloped positive strand RNA virus in the Flaviviridae family. The single strand HCV RNA genome is believed to be approximately 9500 nucleotides in length and has a single open reading frame (ORF) encoding a single large polyprotein of about 3000 amino acids. In infected cells, it is believed that this polyprotein is cleaved at multiple sites by cellular and viral proteases to produce the structural and non-structural (NS) proteins of the virus. In the case of HCV, the generation of mature nonstructural proteins (NS2, NS3, NS4, NS4A, NS4B, NS5A, and NS5B) is believed to be effected by two viral proteases. The first viral protease is believed to cleave at the NS2-NS3 junction of the polyprotein. The second viral protease is believed to be a serine protease contained within the N-terminal region of NS3 (herein referred to as “NS3 protease”). NS3 protease is believed to mediate all of the subsequent cleavage events at sites downstream relative to the position of NS3 in the polyprotein (i.e., sites located between the C-terminus of NS3 and the C-terminus of the polyprotein). NS3 protease exhibits activity both in cis, at the NS3-NS4 cleavage site, and in trans, for the remaining NS4A-NS4B, NS4B-NS5A, and NS5A-NS5B sites. The NS4A protein is believed to serve multiple functions, acting as a cofactor for the NS3 protease and possibly assisting in the membrane localization of NS3 and other viral replicase components. Apparently, the formation of the complex between NS3 and NS4A may be necessary for NS3-mediated processing events and enhances proteolytic efficiency at all sites recognized by NS3. The NS3 protease also appears to exhibit nucleoside triphosphatase and RNA helicase activities. NS5B is believed to be an RNA-dependent RNA polymerase involved in the replication of HCV RNA. In addition, compounds that inhibit the action of NS5A in viral replication are potentially useful for the treatment of HCV.

### SUMMARY OF THE INVENTION

Some embodiments include a compound having the structure of Formula VI:

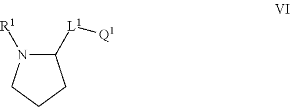

or a pharmaceutically acceptable salt thereof,

wherein:

R¹ is selected from the group consisting of hydrogen, R^{1a}C(═O)— and R^{1a}C(═S)—; each R^{1a} is separately selected from the group consisting of —C(R^{2a})₂NR^{3a}R^{3b}, alkoxyalkyl, C₁₋₆alkylOC(═O)—, C₁₋₆alkylOC(═O)C₁₋₆alkyl, C₁₋₆alkylC(═O)C₁₋₆alkyl, aryl, aryl(CH═CH)ₘ—, arylalkylO—, arylalkyl, arylOalkyl, cycloalkyl, (cycloalkyl)(CH═CH)ₘ—, (cycloalkyl)alkyl, cycloalkylOalkyl, heterocyclyl, heterocyclyl(CH═CH)ₘ—, heterocyclylalkoxy, heterocyclylalkyl, heterocyclylOalkyl, hydroxyalkyl, R^{c}R^{d}N—, (R^{c}R^{d}N)(CH═CH)ₘ—, (R^{c}R^{d}N)alkyl, (R^{c}R^{d}N)C(═O)—, C₁₋₆alkoxy optionally substituted with up to 9 halo, and C₁₋₆alkyl optionally substituted with up to 9 halo;

each R^{c}R^{d}N is separately selected, wherein R^{c} and R^{d} are each independently selected from hydrogen, alkoxyC(═O)—, C₁₋₆alkyl, C₁₋₆alkylC(═O)—, C₁₋₆alkylsulfonyl, arylalkylOC(═O)—, arylalkyl, arylalkylC(═O)—, arylC(═O)—, arylsulfonyl, heterocyclylalkyl, heterocyclylalkylC(═O)—, heterocyclylC(═O)—, (R^{e}R^{f}N)alkyl, (R^{e}R^{f}N)alkylC(═O)—, and (R^{e}R^{f}N)C(═O)—, wherein the alkyl part of arylalkyl, arylalkylC(═O)—, heterocyclylalkyl, and heterocyclylalkylC(═O)— are each optionally substituted with one R^{e}R^{f}N— group; and wherein the aryl part of arylalkyl, arylalkylC(═O)—, arylC(═O)—, and arylsulfonyl, and the heterocyclyl part of heterocyclylalkyl, heterocyclylalkylC(═O)—, and heterocyclylC(═O)— are each optionally substituted with up to three substituents each independently selected from the group consisting of cyano, halo, nitro, C₁₋₆alkoxy optionally substituted with up to 9 halo, and C₁₋₆alkyl optionally substituted with up to 9 halo;

each R^{e}R^{f}N is separately selected, wherein R^{e} and R^{f} are each separately selected from hydrogen, C₁₋₆alkyl, aryl, arylalkyl, cycloalkyl, (cyclolalkyl)alkyl, heterocyclyl, heterocyclylalkyl, (R^{x}R^{y}N)alkyl, and (R^{x}R^{y}N)C(═O)—;

each R^{x}R^{y}N is separately selected, wherein R^{x} and R^{y} are each separately selected from hydrogen, alkylOC(═O)—, alkyl, alkylC(═O)—, aryl, arylalkyl, cycloalkyl, and heterocyclyl;

each R^{2a} is separately selected from the group consisting of hydrogen, C₁₋₆alkyl, aryl(CH₂)ₙ—, and heteroaryl(CH₂)ₙ—;

each R^{3a} is separately selected from the group consisting of hydrogen, and optionally substituted C₁₋₆alkyl;

each R^{3b} is separately selected from the group consisting of optionally substituted C₁₋₆alkyl, heteroaryl, —(CH₂)ₙC(═O)NR^{4a}R^{4b}, —(CH₂)ₙC(═O)OR^{5a}, and —(CH₂)ₙC(═O)R^{6a} said heteroaryl optionally substituted with cyano, halo, nitro, hydroxyl, C₁₋₆alkoxy optionally substituted with up to 9 halo, and C₁₋₆alkyl optionally substituted with up to 9 halo;

each R^{4a}R^{4b}N is separately selected, wherein R^{4a} and R^{4b} are each separately selected from the group consisting of hydrogen, optionally substituted C₁₋₆alkyl, and aryl(CH₂)ₙ—;

each R^{5a} is separately selected from the group consisting of optionally substituted C₁₋₆alkyl, and aryl(CH₂)ₙ—;

each R^{6a} is separately selected from the group consisting of optionally substituted C₁₋₆alkyl, and aryl(CH₂)ₙ—;

L¹ is selected from the group consisting of

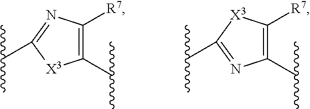

—C(═O)(CH₂)ₘOC(═O)—, —C(CF₃)₂NR^{2c}—, and

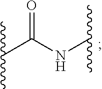

R^{2c} is selected from the group consisting of hydrogen, C₁₋₆alkyl, C₂₋₆alkenyl, C₂₋₆alkynyl, C₃₋₇cycloalkyl, aryl, arylalkyl, heteroaryl, heteroarylalkyl, heterocyclyl, and heterocyclylalkyl, said alkyl optionally substituted with R^{e}R^{f}N—, alkoxy, or C₁₋₆alkylS—;

each X³ is separately selected from the group consisting of NH, NC₁₋₆alkyl, O (oxygen), and S (sulfur);

each m separately is 1 or 2;

each n separately is 0, 1 or 2;

each R⁷ is separately selected from the group consisting of hydrogen, C₁₋₆alkylOC(═O)—, arylalkylOC(═O)—, —COOH, (R^{a}R^{b}N)C(═O)—, trialkylsilylalkylOalkyl, and C₁₋₆alkyl optionally substituted with up to 9 halo;

Q¹ is selected from the group consisting of L² and L³-L⁴.

L² is selected from the group consisting of

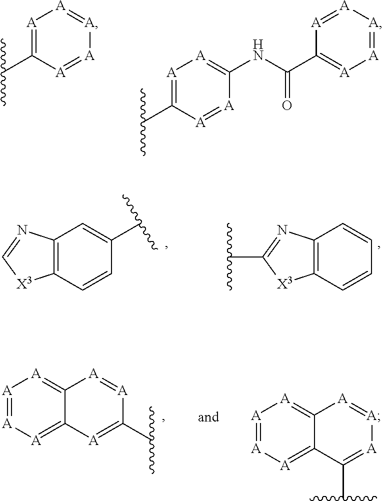

each A is separately selected from the group consisting of CR³ and N (nitrogen);

each R³ is separately selected from the group consisting of hydrogen, C₁₋₆alkoxy, C₁₋₆alkylOC₁₋₆alkyl, C₁₋₆alkylOC(═O)—, arylalkylOC(═O)—, —COOH, halo, hydroxy, R^{a}R^{b}N—, (R^{a}R^{b}N)alkyl, (R^{a}R^{b}N)C(═O)—, and C₁₋₆alkyl optionally substituted with up to 9 halo and up to 5 hydroxy;

each R^{a}R^{b}N is separately selected, wherein R^{a} and R^{b} are each separately selected from the group consisting of hydrogen, C₂₋₆alkenyl, and C₁₋₆alkyl.

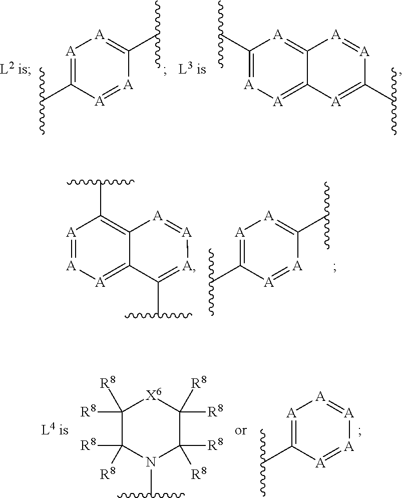

each R⁸ is separately selected from the group consisting of hydrogen, C₁₋₆alkoxy, C₁₋₆alkylOC₁₋₆alkyl, C₁₋₆alkylOC(═O)—, arylalkylOC(═O)—, —COOH, halo, hydroxy, R^{a}R^{b}N—, (R^{a}R^{b}N)alkyl, (R^{a}R^{b}N)C(═O)—, C₁₋₆alkyl optionally substituted with up to 9 halo and C₁₋₆alkyl optionally substituted with up to 5 hydroxy, or optionally two geminal R⁸ are together oxo;

X⁶ is selected from the group consisting of O (oxygen), NR⁹ (nitrogen), and C(R⁸)₂; and

R⁹ is separately selected from the group consisting of hydrogen, aryl(CH₂)ₙ—, C₁₋₆alkylO(CH₂)ₙ, C₁₋₆alkylOC(═O)—, C₁₋₆alkylNHC(═O)—, C₁₋₆alkylC(═O)—, arylC(═O)—, arylOC(═O)—, arylNHC(═O)—, arylalkylOC(═O)—, (R^{a}R^{b}N)(CH₂)ₙ, (R^{a}R^{b}N)C(═O)—, and C₁₋₆alkyl optionally substituted with up to 9 halo, said aryl(CH₂)ₙ—, arylC(═O)—, arylOC(═O)—, and arylNHC(═O)—, each optionally substituted with up to 5 substituents each individually selected from the group consisting of halo, hydroxy, cyano, nitro, C₁₋₆alkyl optionally substituted with up to 9 halo, and C₁₋₆alkoxy optionally substituted with up to 9 halo.

In some embodiments, the compound Formula VI has the structure

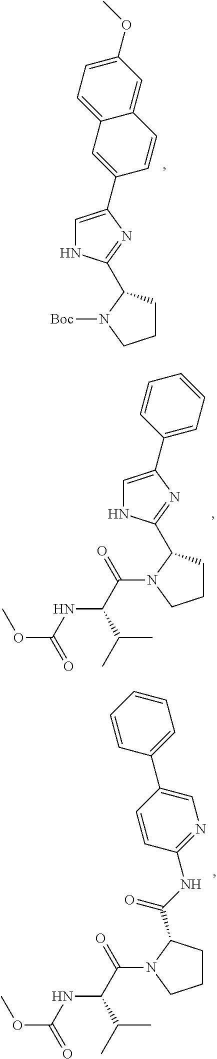

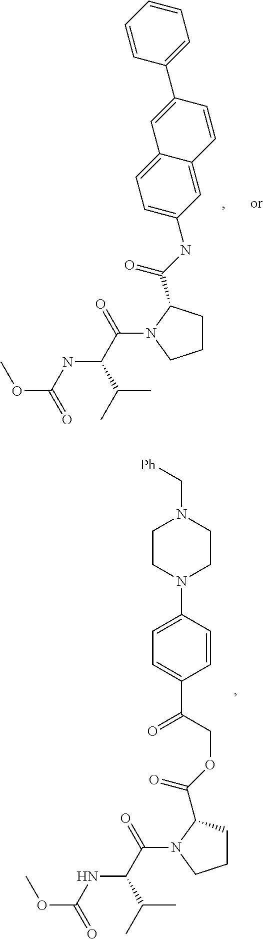

or a pharmaceutically acceptable salt thereof.

Some embodiments include a compound having the structure of Formula VII:

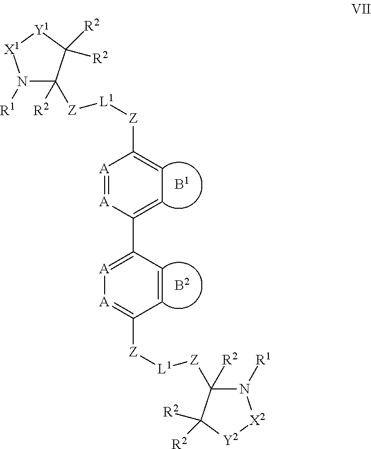

or a pharmaceutically acceptable salt thereof,

wherein:

each R¹ is separately selected from the group consisting of hydrogen, R^{1a}C(═O)— and R^{1a}C(═S)—;

each R^{1a} is separately selected from the group consisting of —C(R^{2a})₂NR^{3a}R^{3b}, alkoxyalkyl, C₁₋₆alkylOC(═O)—, C₁₋₆alkylOC(═O)C₁₋₆alkyl, C₁₋₆alkylC(═O)C₁₋₆alkyl, aryl, aryl(CH₂)ₙ—, aryl(CH₂)ₙO, aryl(CH═CH)ₘ—, arylalkylO—, arylalkyl, arylOalkyl, cycloalkyl, (cycloalkyl)(CH═CH)ₘ—, (cycloalkyl)alkyl, cycloalkylOalkyl, heterocyclyl, heterocyclyl(CH═CH)ₘ—, heterocyclylalkoxy, heterocyclylalkyl, heterocyclylOalkyl, hydroxyalkyl, R^{c}R^{d}N—, R^{c}R^{d}N(CH₂)ₙ—, (R^{c}R^{d}N)(CH═CH)ₘ—, (R^{c}R^{d}N)alkyl, (R^{c}R^{d}N)C(═O)—, C₁₋₆alkoxy optionally substituted with up to 9 halo, and C₁₋₆alkyl optionally substituted with up to 9 halo, said aryl and heteroaryl each optionally substituted with cyano, halo, nitro, hydroxyl, C₁₋₆alkoxy optionally substituted with up to 9 halo, and C₁₋₆alkyl optionally substituted with up to 9 halo;

each R^{c}R^{d}N is separately selected, wherein R^{c} and R^{d} are each separately selected from the group consisting of hydrogen, alkoxyC(═O)—, C₁₋₆alkyl, C₁₋₆alkylC(═O)—, C₁₋₆alkylsulfonyl, arylalkylOC(═O)—, arylalkyl, arylalkylC(═O)—, arylC(═O)—, arylsulfonyl, heterocyclylalkyl, heterocyclylalkylC(═O)—, heterocyclylC(═O)—, (R^{e}R^{f}N)alkyl, (R^{e}R^{f}N)alkylC(═O)—, and (R^{e}R^{f}N)C(═O)—, wherein the alkyl part of arylalkyl, arylalkylC(═O)—, heterocyclylalkyl, and heterocyclylalkylC(═O)— are each optionally substituted with one R^{e}R^{f}N— group; and wherein the aryl part of arylalkyl, arylalkylC(═O)—, arylC(═O)—, and arylsulfonyl, and the heterocyclyl part of heterocyclylalkyl, heterocyclylalkylC(═O)—, and heterocyclylC(═O)— are each optionally substituted with up to three substituents each independently selected from the group consisting of cyano, halo, nitro, C₁₋₆alkoxy optionally substituted with up to 9 halo, and C₁₋₆alkyl optionally substituted with up to 9 halo;

each R^{e}R^{f}N is separately selected, wherein R^{e} and R^{f} are each separately selected from the group consisting of hydrogen, C₁₋₆alkyl, aryl, arylalkyl, cycloalkyl, (cyclolalkyl)alkyl, heterocyclyl, heterocyclylalkyl, (R^{x}R^{y}N)alkyl, and (R^{x}R^{y}N)C(═O)—;

each R^{x}R^{y}N is separately selected, wherein R^{x} and R^{y} are each separately selected from the group consisting of hydrogen, alkylOC(═O)—, C₁₋₆alkyl, C₁₋₆alkylC(═O)—, aryl, arylalkyl, cycloalkyl, and heterocyclyl;

each C(R^{2a})₂ is separately selected, wherein each R^{2a} is separately selected from the group consisting of hydrogen, C₁₋₆alkyl optionally substituted with up to 9 halo, aryl(CH₂)ₙ—, and heteroaryl(CH₂)ₙ—, said aryl and heteroaryl each optionally substituted with cyano, halo, nitro, hydroxyl, C₁₋₆alkoxy optionally substituted with up to 9 halo, and C₁₋₆alkyl optionally substituted with up to 9 halo, or C(R^{2a})₂ is

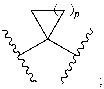

each R^{3a} is separately selected from the group consisting of hydrogen, and optionally substituted C₁₋₆alkyl;

each R^{3b} is separately selected from the group consisting of optionally substituted said heteroaryl optionally substituted with cyano, halo, nitro, hydroxyl, C₁₋₆alkoxy optionally substituted with up to 9 halo, and C₁₋₆alkyl optionally substituted with up to 9 halo;

each R^{4a}R^{4b}N is separately selected, wherein R^{4a} and R^{4b} are each separately selected from the group consisting of hydrogen, optionally substituted C₁₋₆alkyl, and aryl(CH₂)ₙ—;

each R^{5a} is separately selected from the group consisting of optionally substituted C₁₋₆alkyl, and aryl(CH₂)ₙ—;

each R^{6a} is separately selected from the group consisting of optionally substituted C₁₋₆alkyl, and aryl(CH₂)ₙ—;

X¹ is (C(R²)₂)_{q},

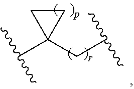

or X¹ is null;

Y¹ is selected from O (oxygen), S (sulfur), S(O), SO₂, NR², and C(R²)₂ with the proviso that when X¹ is null Y¹ is C(R²)₂;

X² is (C(R²)₂)_{q},

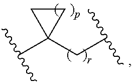

or X² is null;

Y² is selected from O (oxygen), S (sulfur), S(O), SO₂, NR², and C(R²)₂ with the proviso that when X² is null Y² is C(R²)₂;

each R² is separately selected, wherein R² is selected from the group consisting of hydrogen, C₁₋₆alkoxy, C₁₋₆alkyl, aryl, halo, hydroxy, R^{a}R^{b}N—, and C₁₋₆alkyl optionally substituted with up to 9 halo, or optionally two vicinal R² and the carbons to which they are attached are together a fused three- to six-membered carbocyclic ring optionally substituted with up to two C₁₋₆alkyl groups;

each A is separately selected from the group consisting of CR³ and N (nitrogen);

each R³ is separately selected from the group consisting of hydrogen, C₁₋₆alkoxy, C₁₋₆alkylOC₁₋₆alkyl, C₁₋₆alkylOC(═O)—, arylalkylOC(═O)—, —COOH, halo, hydroxy, R^{a}R^{b}N—, (R^{a}R^{b}N)alkyl, (R^{a}R^{b}N)C(═O)—, and C₁₋₆alkyl optionally substituted with up to 9 halo and up to 5 hydroxy;

each L¹ is separately selected from the group consisting of

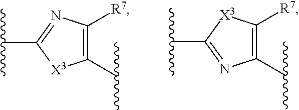

—C(═O)(CH₂)ₘOC(═O)—, —C(CF₃)₂NR^{2c}—, and

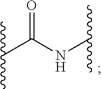

R^{2c} is selected from the group consisting of hydrogen, C₁₋₆alkyl, C₂₋₆alkenyl, C₂₋₆alkynyl, C₃₋₇cycloalkyl, aryl, arylalkyl, heteroaryl, heteroarylalkyl, heterocyclyl, and heterocyclylalkyl, said alkyl optionally substituted with R^{e}R^{f}N—, alkoxy, or C₁₋₆alkylS—;

each X³ is separately selected from the group consisting of NH, NC₁₋₆alkyl, O (oxygen), and S (sulfur);

each R⁷ is separately selected from the group consisting of hydrogen, C₁₋₆alkylOC(═O)—, arylalkylOC(═O)—, —COOH, (R^{a}R^{b}N)C(═O)—, trialkylsilylalkylOalkyl, and C₁₋₆alkyl optionally substituted with up to 9 halo;

each R^{a}R^{b}N is separately selected, wherein R^{a} and R^{b} are each separately selected from the group consisting of hydrogen, C₂₋₆alkenyl, and C₁₋₆alkyl;

each Z is separately selected, wherein Z is selected from the group consisting of O (oxygen) and CH₂, or Z is null;

each m separately is 1 or 2;

each n separately is 0, 1 or 2;

each p separately is 1, 2, 3 or 4;

each q separately is 1, 2, 3, 4 or 5;

each r separately is 0, 1, 2, 3, or 4;

B¹ is a fused optionally substituted saturated or unsaturated three- to seven-membered carbocyclic ring or a fused optionally substituted saturated or unsaturated three- to seven-membered heterocyclic ring, each optionally substituted with one or more R⁴;

B² is a fused optionally substituted saturated or unsaturated three- to seven-membered carbocyclic ring or a fused optionally substituted saturated or unsaturated three- to seven-membered heterocyclic ring, each optionally substituted with one or more R⁴; and

each R⁴ is separately selected from the group consisting of C₁₋₆alkoxy, C₁₋₆alkylOC₁₋₆alkyl, C₁₋₆alkylOC(═O)—, arylalkylOC(═O)—, —COOH, halo, C₁₋₆haloalkyl, hydroxy, R^{a}R^{b}N—, (R^{a}R^{b}N)alkyl, (R^{a}R^{b}N)C(═O)—, and C₁₋₆alkyl optionally substituted with up to 9 halo and up to 5 hydroxy, or optionally two geminal R⁴ are together oxo.

In some embodiments of Formula VII,

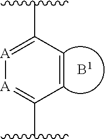

is selected from the group consisting of:

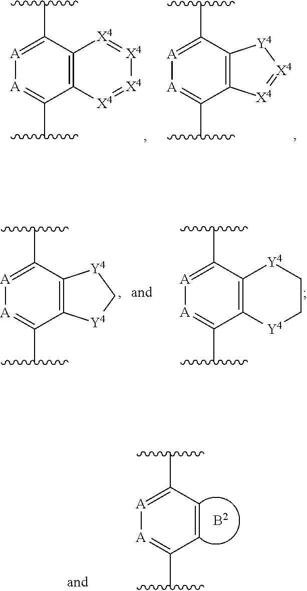

is selected from the group consisting of:

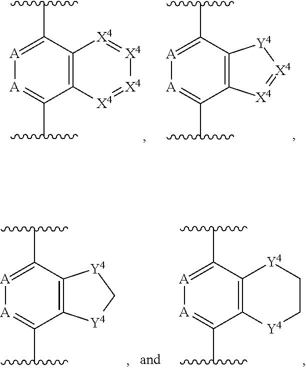

wherein,

each X⁴ is separately selected from the group consisting of CR⁴ and N (nitrogen); and

each Y⁴ is separately selected from the group consisting of C(R⁴)₂, NR⁴, O (oxygen), and S (sulfur). In some embodiments of Formula VII, each Z is null.

In some embodiments of Formula VII, B¹ is a fused saturated or unsaturated three- to seven-membered carbocyclic ring optionally substituted with one or more R⁴. In some embodiments of Formula VII, B² is a fused saturated or unsaturated three- to seven-membered carbocyclic ring optionally substituted with one or more R⁴. In some embodiments of Formula VII, B¹ is a fused saturated or unsaturated three- to seven-membered heterocyclic ring optionally substituted with one or more R⁴. In some embodiments of Formula VII, B² is a fused saturated or unsaturated three- to seven-membered heterocyclic ring optionally substituted with one or more R⁴.

In some embodiments of Formula VII,

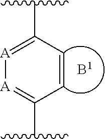

is selected from the group consisting of:

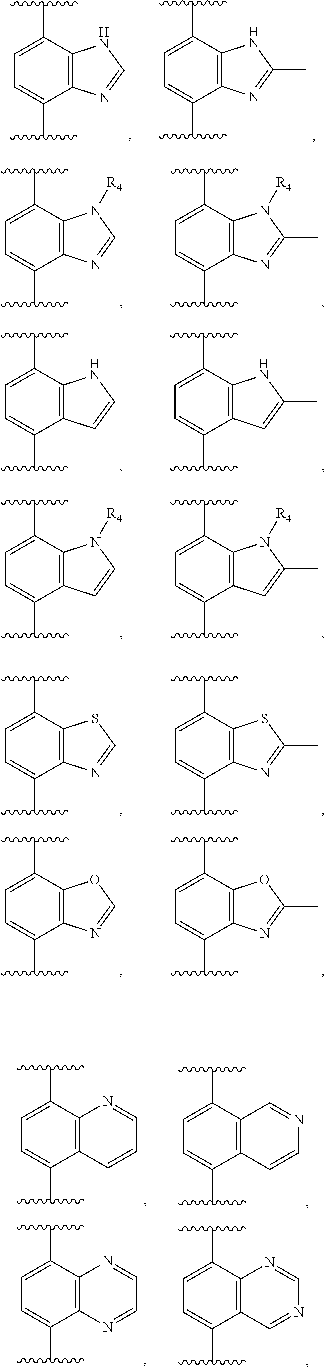

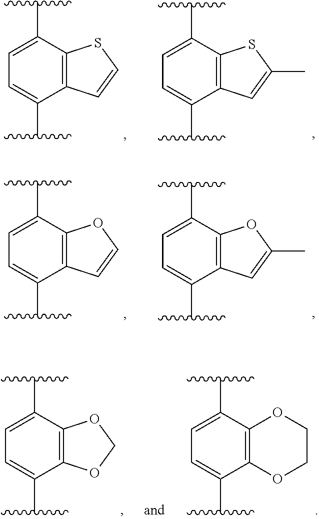

In some embodiments of Formula VII,

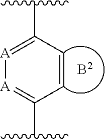

is selected from the group consisting of:

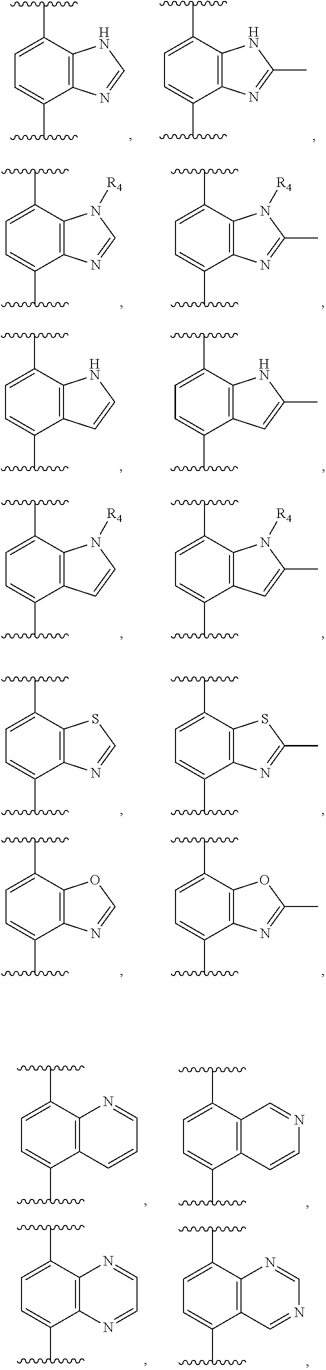

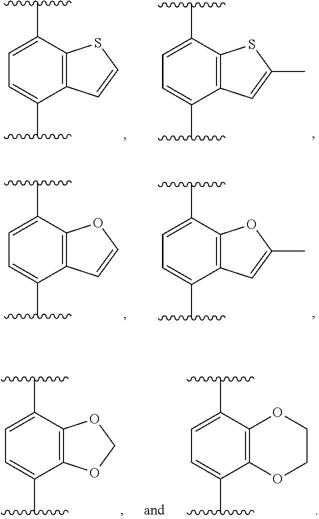

In some embodiments of Formula VII,

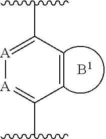

is selected from the group consisting of:

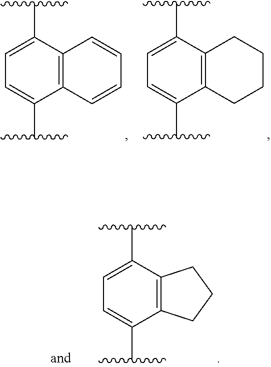

In some embodiments of Formula VII,

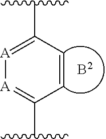

is selected from the group consisting of:

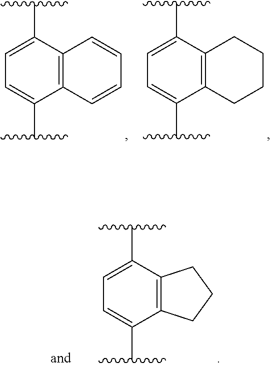

In some embodiments, the compound of Formula VII has the structure of Formula VIIa:

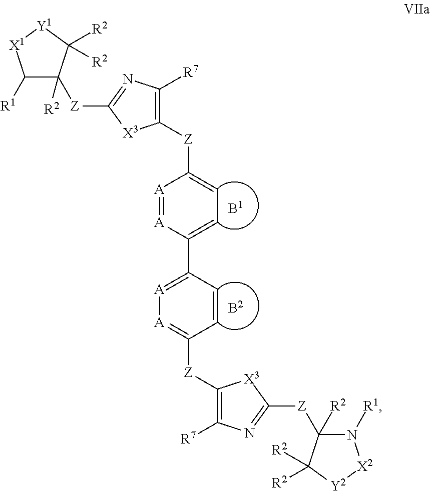

or a pharmaceutically acceptable salt thereof.

In some embodiments, the compound of Formula VII has the structure of Formula VIIb:

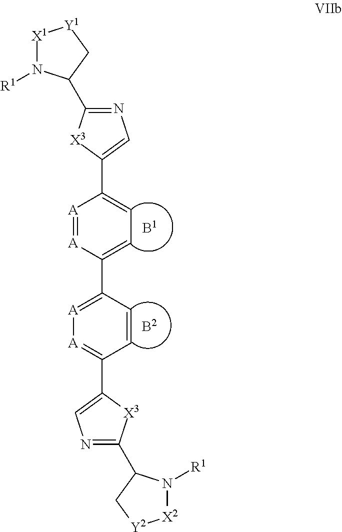

or a pharmaceutically acceptable salt thereof.

In some embodiments of Formula VIIb, each R¹ is R^{1a}C(═O)—. In some embodiments of Formula VIIb, each R^{1a} is —CHR^{2a}NHR^{3b}. In some embodiments of Formula VIIb, each R^{2a} is C₁₋₆alkyl; each R^{3b} is —C(═O)OR⁵; and each R⁵ is C₁₋₆alkyl.

In some embodiments, the compound Formula VIIb has the structure

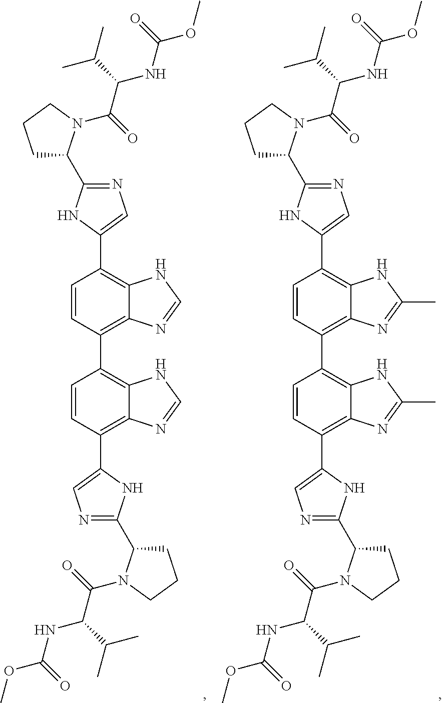

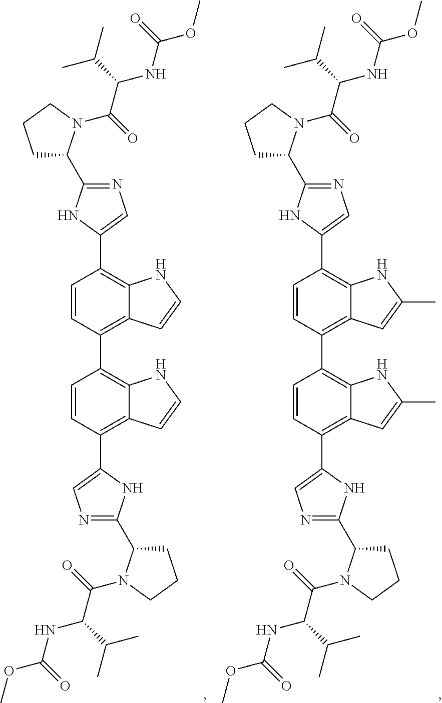

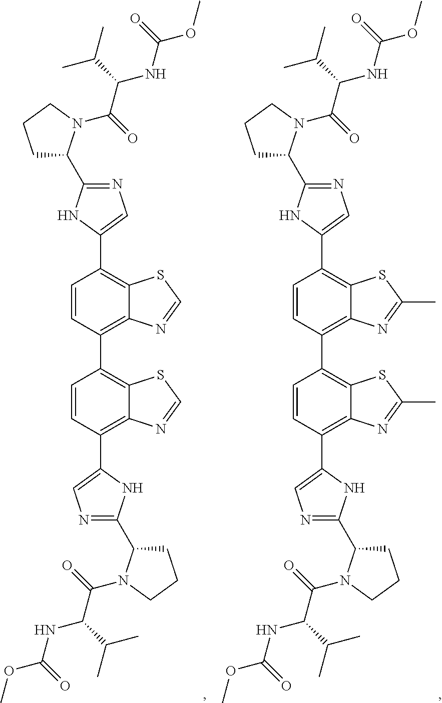

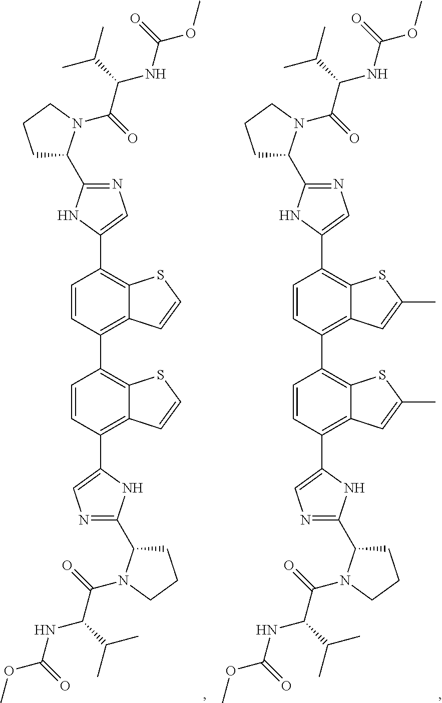

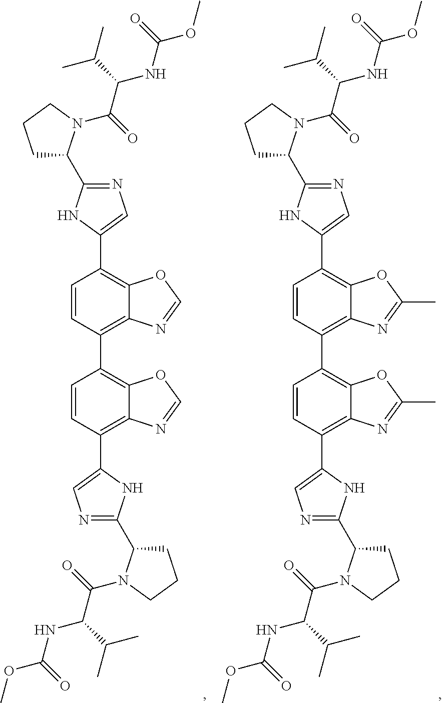

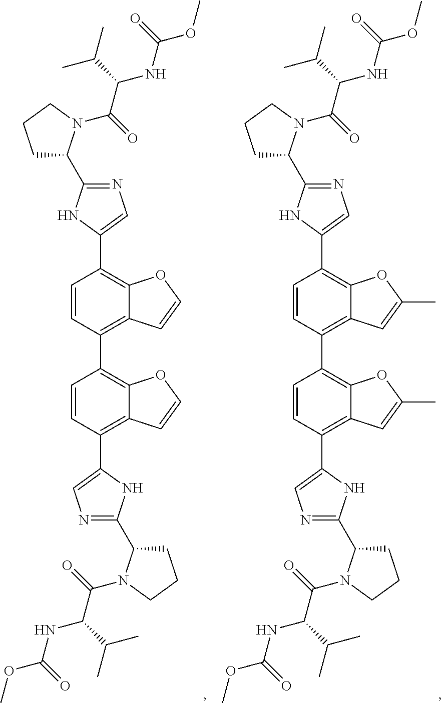

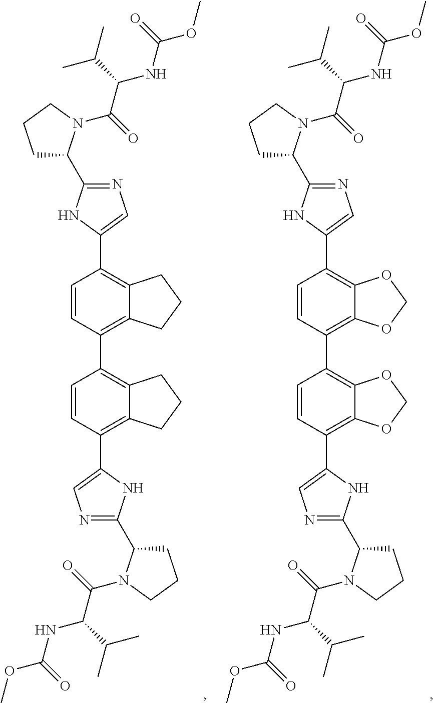

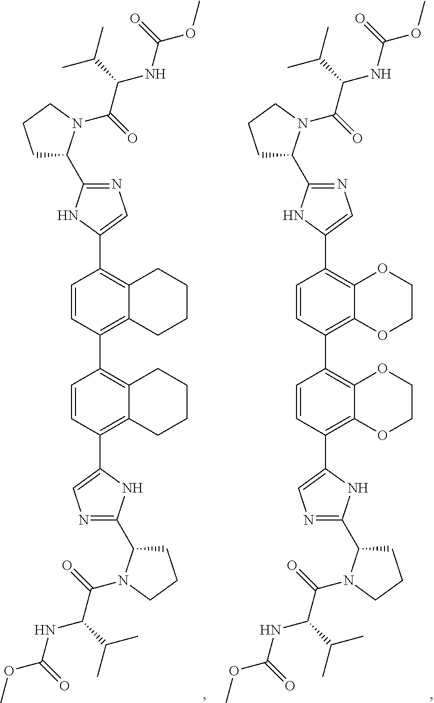

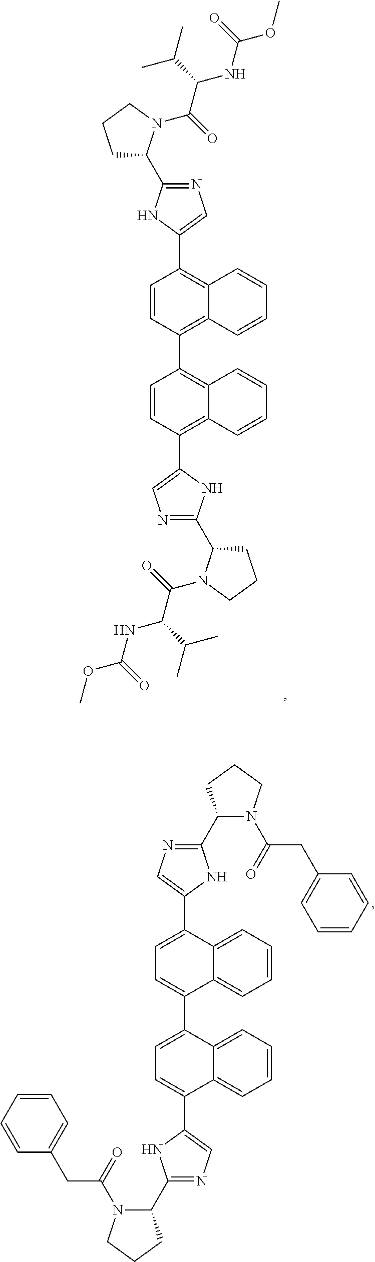

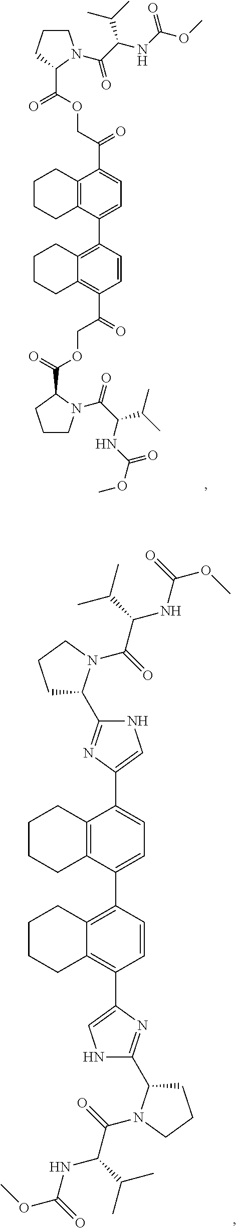

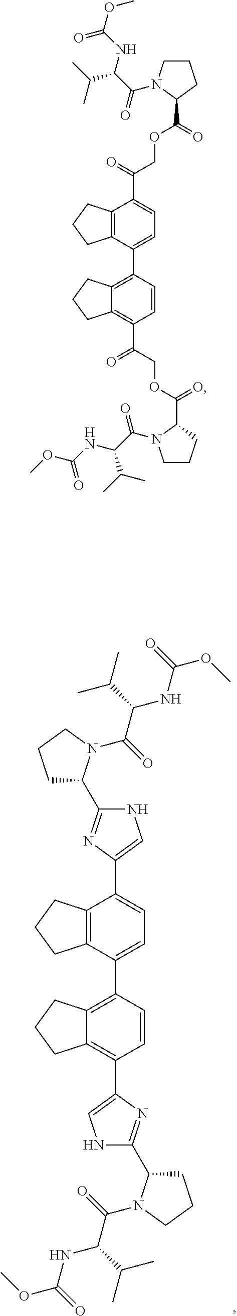

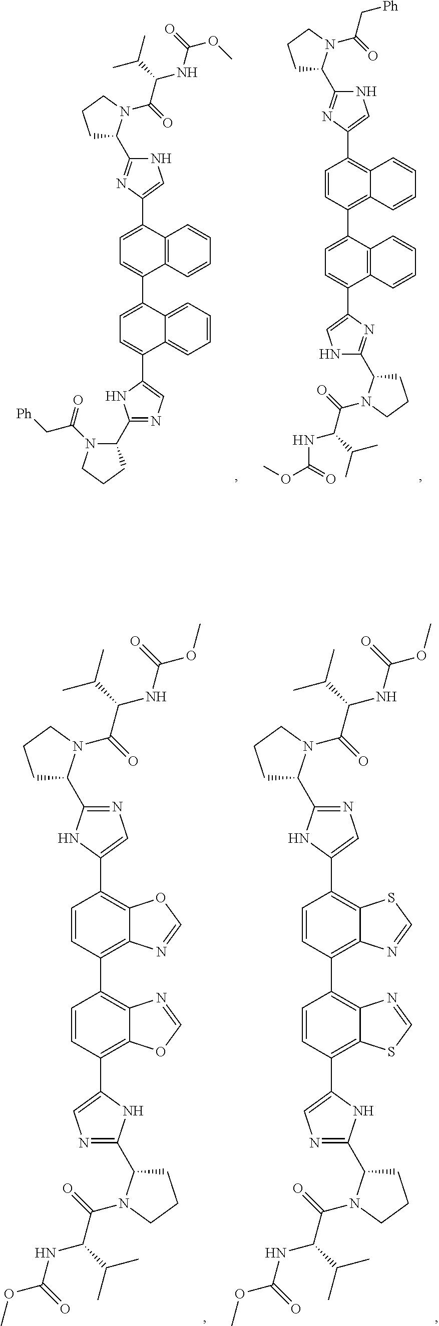

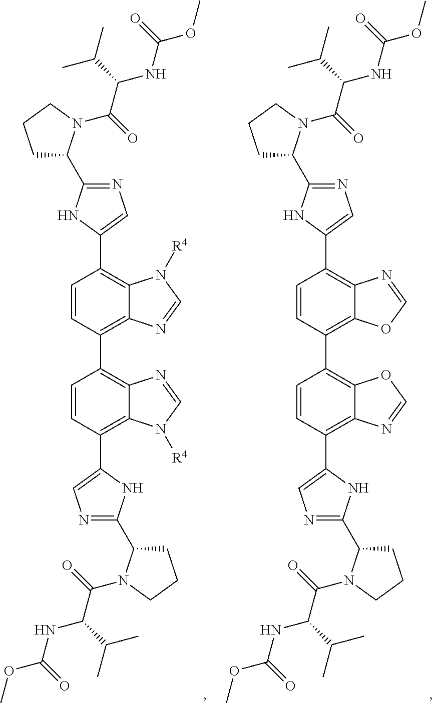

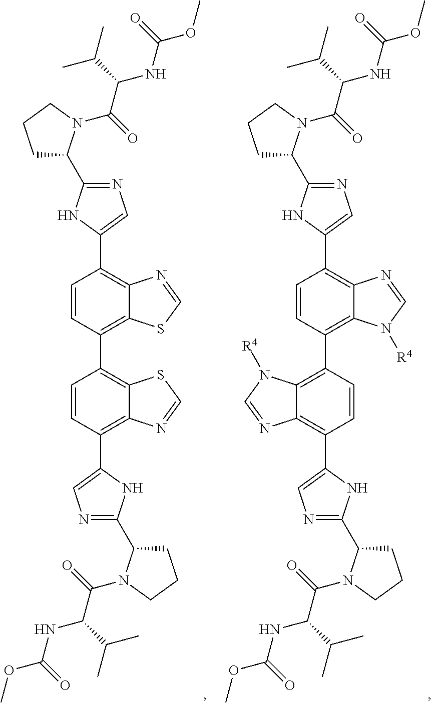

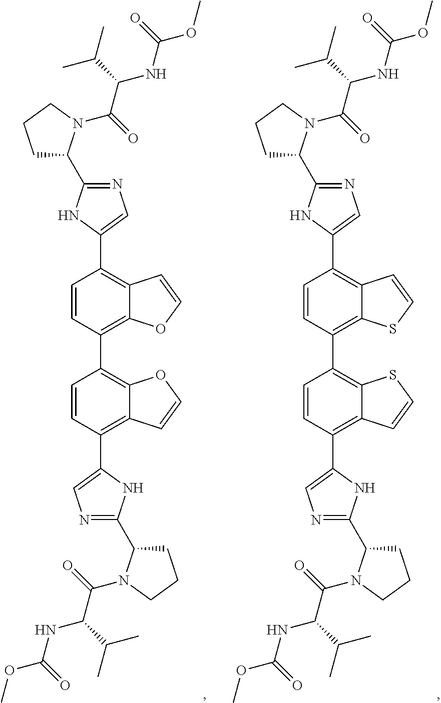

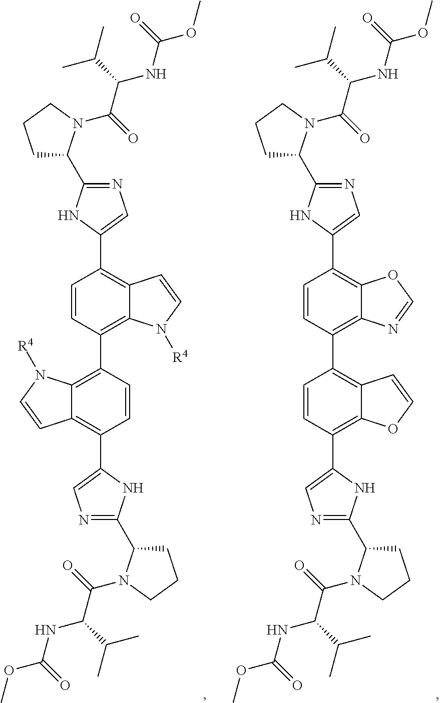

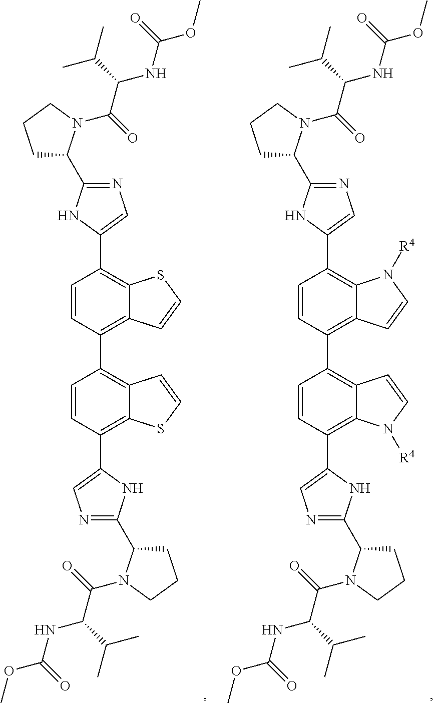

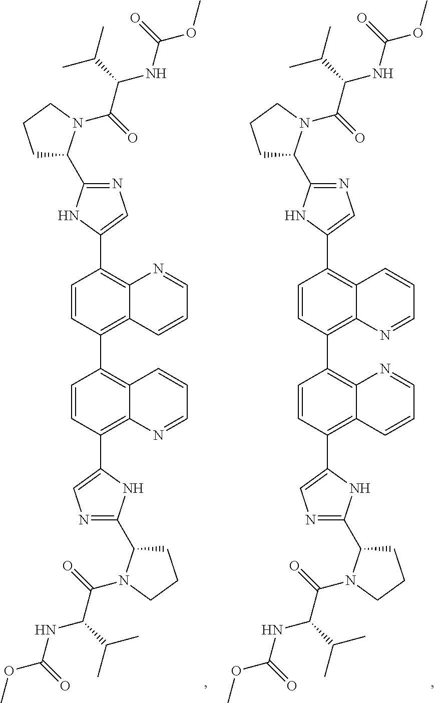

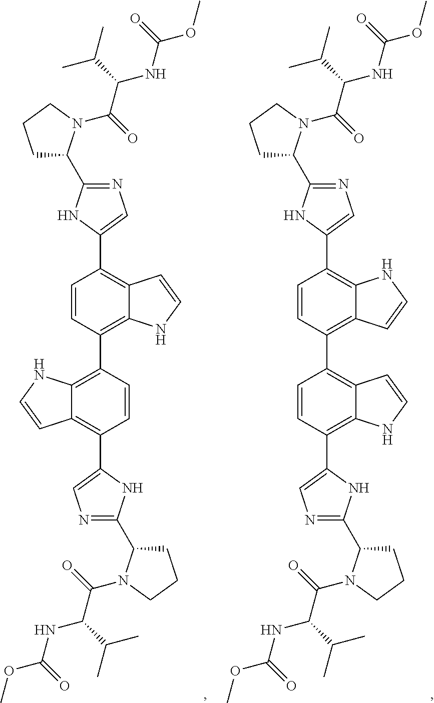

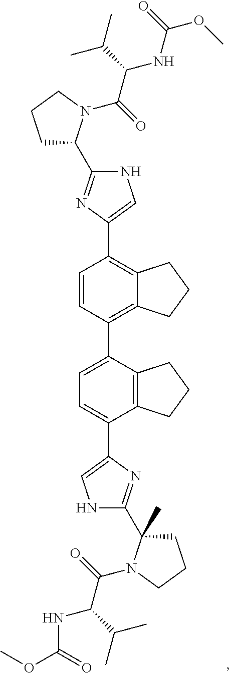

or a pharmaceutically acceptable salt thereof.

In some embodiments, the compound of Formula VII has the structure of Formula VIIc:

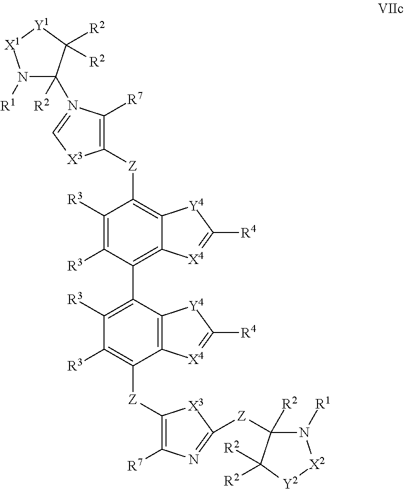

or a pharmaceutically acceptable salt thereof wherein:

each X⁴ is separately selected from the group consisting of CR⁴ and N (nitrogen); and

each Y⁴ is separately selected from the group consisting of C(R⁴)₂, NR⁴, O (oxygen), and S (sulfur).

In some embodiments, the compound of Formula VII has the structure of Formula VIId:

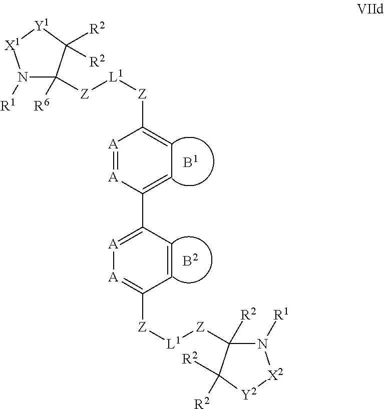

or a pharmaceutically acceptable salt thereof, wherein R⁶ is C₁₋₆alkyl optionally substituted with up to 9 halo. In some embodiments, R⁶ is methyl.

Some embodiments include a compound having the structure of Formula VIII:

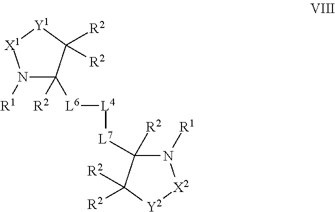

or a pharmaceutically acceptable salt thereof,

wherein:

each R¹ is separately selected from the group consisting of hydrogen, R^{1a}C(═O)— and R^{1a}C(═S)—;

each R^{1a} is separately selected from the group consisting of —C(R^{2a})₂NR^{3a}R^{3b}, alkoxyalkyl, C₁₋₆alkylOC(═O)—, C₁₋₆alkylOC(═O)C₁₋₆alkyl, C₁₋₆alkylC(═O)C₁₋₆alkyl, aryl, aryl(CH₂)ₙ—, aryl(CH₂)ₙO—, aryl(CH═CH)ₘ—, arylalkylO—, arylalkyl, arylOalkyl, cycloalkyl, (cycloalkyl)(CH═CH)ₘ—, (cycloalkyl)alkyl, cycloalkylOalkyl, heterocyclyl, heterocyclyl(CH═CH)ₘ—, heterocyclylalkoxy, heterocyclylalkyl, heterocyclylOalkyl, hydroxyalkyl, R^{c}R^{d}N—, R^{c}R^{d}N(CH₂)ₙ—, (R^{c}R^{d}N)(CH═CH)ₘ—, (R^{c}R^{d}N)alkyl, (R^{c}R^{d}N)C(═O)—, C₁₋₆alkoxy optionally substituted with up to 9 halo, and C₁₋₆alkyl optionally substituted with up to 9 halo, said aryl and heteroaryl each optionally substituted with cyano, halo, nitro, hydroxyl, C₁₋₆alkoxy optionally substituted with up to 9 halo, and C₁₋₆alkyl optionally substituted with up to 9 halo;

each R^{c}R^{d}N is separately selected, wherein R^{c} and R^{d} are each separately selected from hydrogen, alkoxyC(═O)—, C₁₋₆alkyl, C₁₋₆alkylC(═O)—, C₁₋₆alkylsulfonyl, arylalkylOC(═O)—, arylalkyl, arylalkylC(═O)—, arylC(═O)—, arylsulfonyl, heterocyclylalkyl, heterocyclylalkylC(═O)—, heterocyclylC(═O)—, (R^{e}R^{f}N)alkyl, (R^{e}R^{f}N)alkylC(═O)—, and (R^{e}R^{f}N)C(═O)—, wherein the alkyl part of arylalkyl, arylalkylC(═O)—, heterocyclylalkyl, and heterocyclylalkylC(═O)— are each optionally substituted with one R^{e}R^{f}N— group; and wherein the aryl part of arylalkyl, arylalkylC(═O)—, arylC(═O)—, and arylsulfonyl, and the heterocyclyl part of heterocyclylalkyl, heterocyclylalkylC(═O)—, and heterocyclylC(═O)— are each optionally substituted with up to three substituents each independently selected from the group consisting of cyano, halo, nitro, C₁₋₆alkoxy optionally substituted with up to 9 halo, and C₁₋₆alkyl optionally substituted with up to 9 halo;

each R^{e}R^{f}N is separately selected, wherein R^{e} and R^{f} are each separately selected from hydrogen, C₁₋₆alkyl, aryl, arylalkyl, cycloalkyl, (cyclolalkyl)alkyl, heterocyclyl, heterocyclylalkyl, (R^{x}R^{y}N)alkyl, and (R^{x}R^{Y}N)C(═O)—;

each R^{x}R^{y}N is separately selected, wherein R^{x} and R^{y} are each separately selected from hydrogen, C₁₋₆alkylOC(═O)—, C₁₋₆alkyl, C₁₋₆alkylC(═O)—, aryl, arylalkyl, cycloalkyl, and heterocyclyl;

each C(R^{2a})₂ is separately selected, wherein each R^{2a} is separately selected from the group consisting of hydrogen, C₁₋₆alkyl optionally substituted with up to 9 halo, aryl(CH₂)ₙ—, and heteroaryl(CH₂)ₙ—, said aryl and heteroaryl each optionally substituted with cyano, halo, nitro, hydroxyl, C₁₋₆alkoxy optionally substituted with up to 9 halo, and C₁₋₆alkyl optionally substituted with up to 9 halo, or C(R^{2a})₂ is

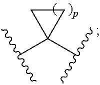

each R^{3a} is separately selected from the group consisting of hydrogen, and optionally substituted C₁₋₆alkyl;

each R^{3b} is separately selected from the group consisting of optionally substituted C₁₋₆alkyl, heteroaryl, —(CH₂)ₙC(═O)NR^{4a}R^{4b}, —(CH₂)ₙC(═O)OR^{5a}, and —(CH₂)ₙC(═O)R^{6a} said heteroaryl optionally substituted with cyano, halo, nitro, hydroxyl, C₁₋₆alkoxy optionally substituted with up to 9 halo, and C₁₋₆alkyl optionally substituted with up to 9 halo;

each R^{4a}R^{4b}N is separately selected, wherein R^{4a} and R^{4b} are each separately selected from the group consisting of hydrogen, optionally substituted C₁₋₆alkyl, and aryl(CH₂)ₙ—;

each R^{5a} is separately selected from the group consisting of optionally substituted C₁₋₆alkyl, and aryl(CH₂)ₙ—;

each R^{6a} is separately selected from the group consisting of optionally substituted C₁₋₆alkyl, and aryl(CH₂)ₙ—;

X¹ is (C(R²)₂)_{q},

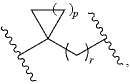

or X¹ is null;

Y¹ is selected from O (oxygen), S (sulfur), S(O), SO₂, NR², and C(R²)₂ with the proviso that when X¹ is null Y¹ is C(R²)₂;

X² is (C(R²)₂)_{q},

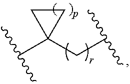

or X² is null;

Y² is selected from O (oxygen), S (sulfur), S(O), SO₂, NR², and C(R²)₂ with the proviso that when X² is null Y² is C(R²)₂;

each R² is separately selected, wherein R² is selected from the group consisting of hydrogen, C₁₋₆alkoxy, C₁₋₆alkyl, aryl, halo, hydroxy, R^{a}R^{b}N—, and C₁₋₆alkyl optionally substituted with up to 9 halo, or optionally two vicinal R² and the carbons to which they are attached are together a fused three- to six-membered carbocyclic ring optionally substituted with up to two C₁₋₆alkyl groups;

each R^{a}R^{b}N is separately selected, wherein R^{a} and R^{b} are each separately selected from the group consisting of hydrogen, C₂₋₆alkenyl, and C₁₋₆alkyl;

L⁴ is selected from the group consisting of

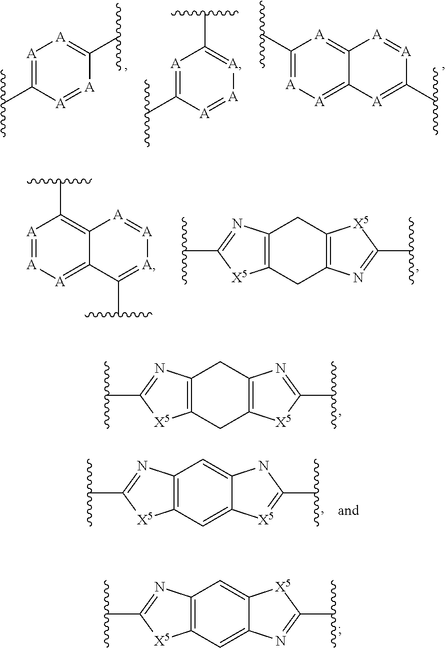

each X⁵ is separately selected from the group consisting of —NH—, O (oxygen), S (sulfur), and —CH₂—;

each A is separately selected from the group consisting of CR³ and N (nitrogen);

each R³ is separately selected from the group consisting of hydrogen, C₁₋₆alkoxy, C₁₋₆alkylOC₁₋₆alkyl, C₁₋₆alkylOC(═O)—, arylalkylOC(═O)—, —COOH, halo, hydroxy, R^{a}R^{b}N—, (R^{a}R^{b}N)alkyl, (R^{a}R^{b}N)C(═O)—, C₁₋₆alkyl optionally substituted with up to 9 halo and C₁₋₆alkyl optionally substituted with up to 5 hydroxy;

L⁶ is selected from the group consisting of

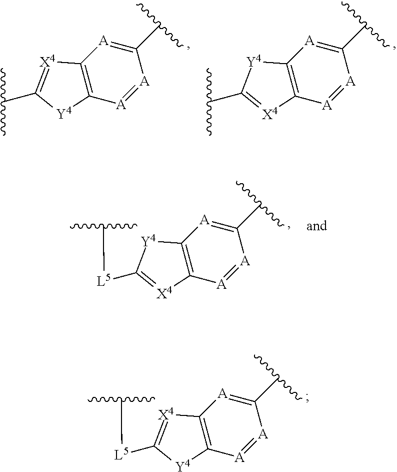

L⁷ is selected from the group consisting of

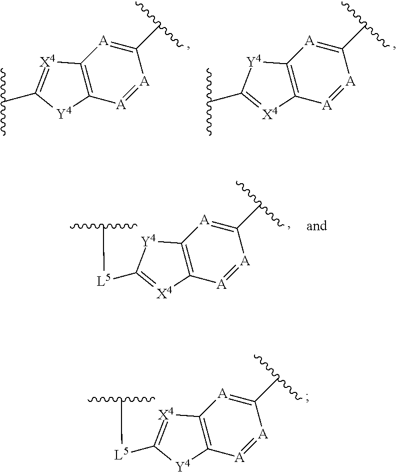

each L⁵ is separately selected from the group consisting of

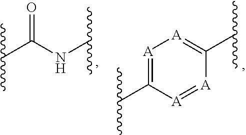

### and —(CH═CH)—;

each X⁴ is separately selected from the group consisting of CR⁴ and N (nitrogen);

each Y⁴ is separately selected from the group consisting of C(R⁴)₂, NR⁴, O (oxygen), and S (sulfur);

each R⁴ is separately selected from the group consisting of C₁₋₆alkoxy, C₁₋₆alkylOC₁₋₆alkyl, C₁₋₆alkylOC(═O)—, arylalkylOC(═O)—, —COOH, halo, C₁₋₆haloalkyl, hydroxy, R^{a}R^{b}N—, (R^{a}R^{b}N)alkyl, (R^{a}R^{b}N)C(═O)—, C₁₋₆alkyl optionally substituted with up to 9 halo and C₁₋₆alkyl optionally substituted with up to 5 hydroxy, or optionally two geminal R⁴ are together oxo;

each m separately is 1 or 2;

each n separately is 0, 1 or 2;

each p separately is 1, 2, 3 or 4;

each q separately is 1, 2, 3, 4 or 5; and

each r separately is 0, 1, 2, 3, or 4,

with the proviso that L⁶-L⁴-L⁷ is not

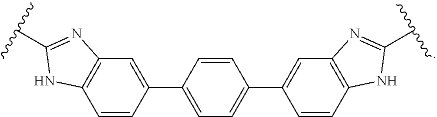

In some embodiments of Formula VIII, each R¹ is R^{1a}C(═O)—. In some embodiments of Formula VIII, each R^{1a} is —CHR^{2a}NHR^{3b}. In some embodiments of Formula VIII, each R^{2a} is C₁₋₆alkyl; each R^{3b} is —C(═O)OR⁵; and each R⁵ is C₁₋₆alkyl. In some embodiments of Formula VIII, L⁶-L⁴-L⁷ is selected from the group consisting of

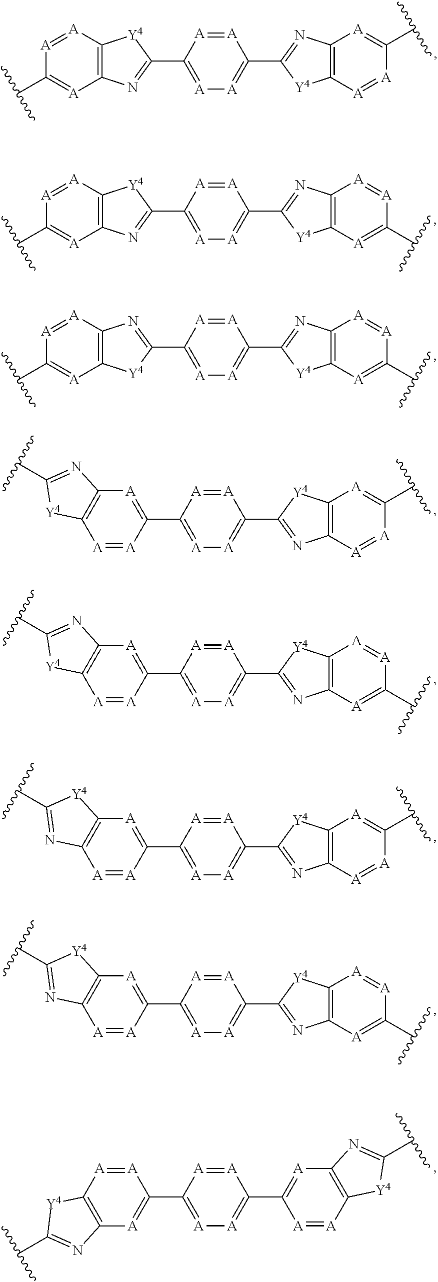

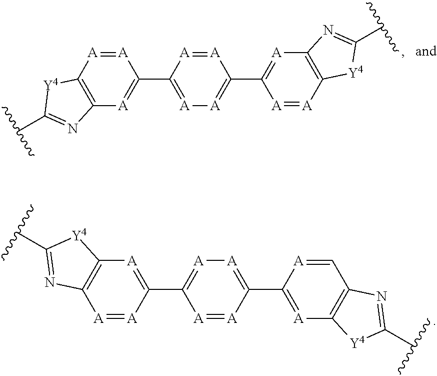

In some embodiments of Formula VIII, each L⁶ is selected from the group consisting of

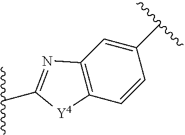

and

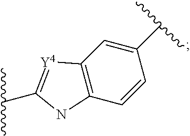

L⁷ is selected from the group consisting of

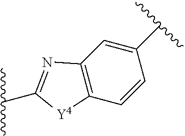

and

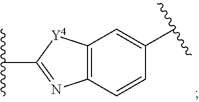

### and L⁴ is

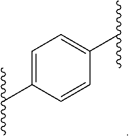

In some embodiments of Formula VIII the compound is not selected from the group consisting of:

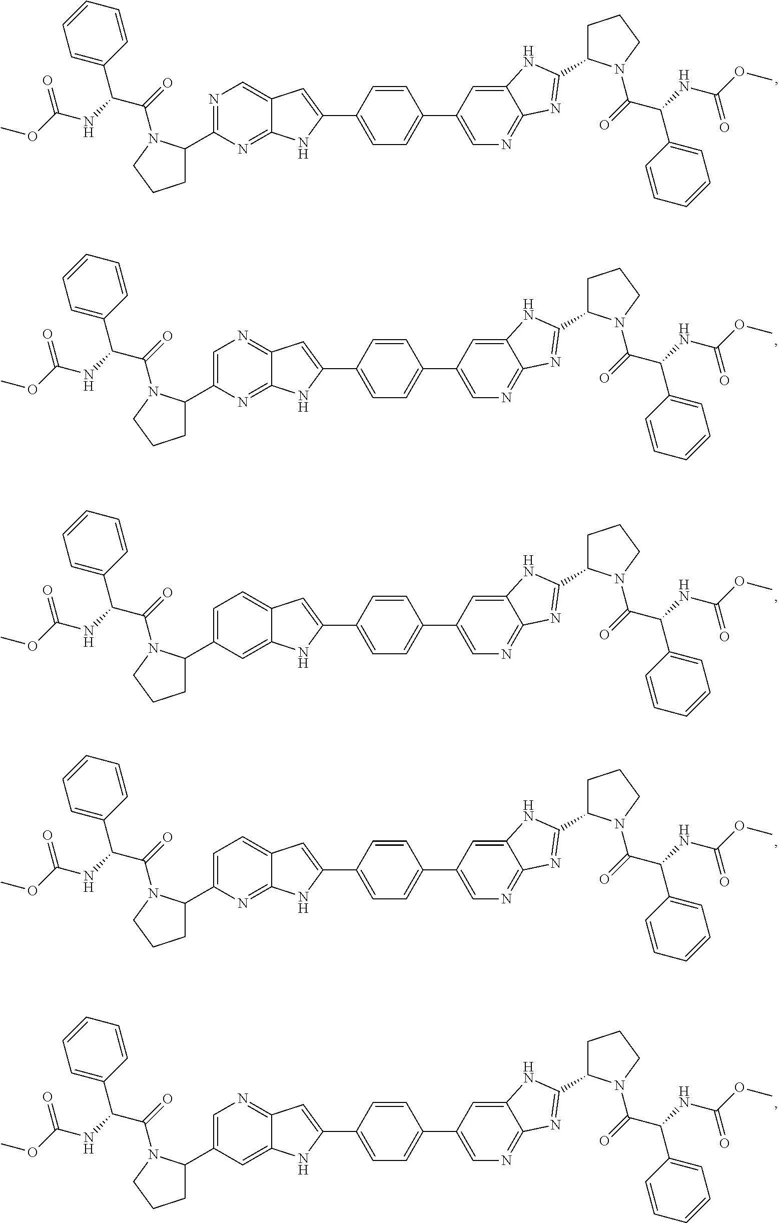

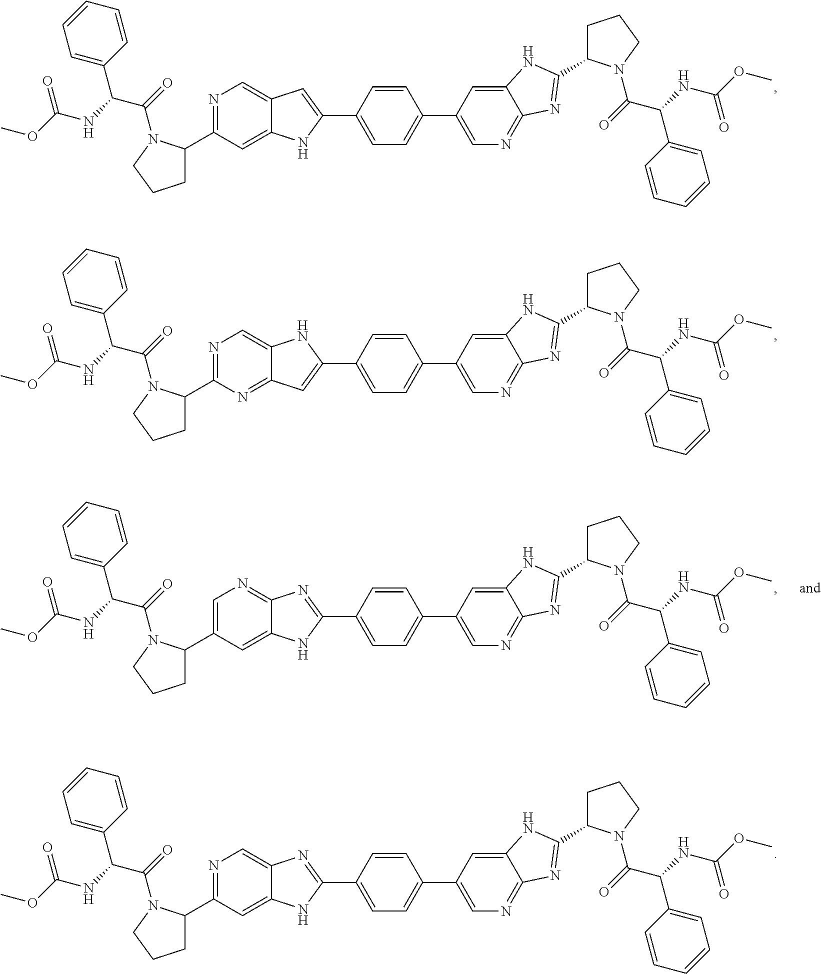

In some embodiments, the compound of Formula VIII has the structure of Formula VIIIa:

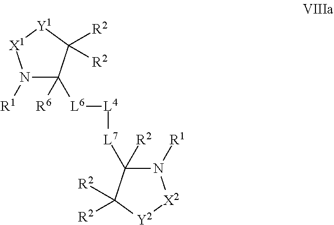

or a pharmaceutically acceptable salt thereof, wherein R⁶ is C₁₋₆alkyl optionally substituted with up to 9 halo. In some embodiments, R⁶ is methyl.

Some embodiments include a compound having the structure of Formula IX:

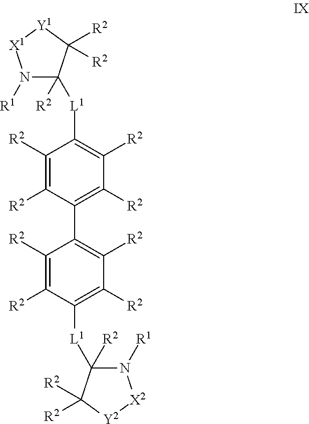

or a pharmaceutically acceptable salt thereof,

wherein:

each R¹ is separately selected from the group consisting of hydrogen, R^{1a}C(═O)— and R^{1a}C(═S)—;

each R^{1a} is separately selected from the group consisting of —C(R^{2a})₂NR^{3a}R^{3b}, alkoxyalkyl, C₁₋₆alkylOC(═O)—, C₁₋₆alkylOC(═O)C₁₋₆alkyl, C₁₋₆alkylC(═O)C₁₋₆alkyl, aryl, aryl(CH₂)ₙ—, aryl(CH₂)ₙO—, aryl(CH═CH)ₘ—, arylalkylO—, arylalkyl, arylOalkyl, cycloalkyl, (cycloalkyl)(CH═CH)ₘ—, (cycloalkyl)alkyl, cycloalkylOalkyl, heterocyclyl, heterocyclyl(CH═CH)ₘ—, heterocyclylalkoxy, heterocyclylalkyl, heterocyclylOalkyl, hydroxyalkyl, R^{c}R^{d}N—, R^{c}R^{d}N(CH₂)ₙ—, (R^{c}R^{d}N)(CH═CH)ₘ—, (R^{c}R^{d}N)alkyl, (R^{c}R^{d}N)C(═O)—, C₁₋₆alkoxy optionally substituted with up to 9 halo, and C₁₋₆alkyl optionally substituted with up to 9 halo, said aryl and heteroaryl each optionally substituted with cyano, halo, nitro, hydroxyl, C₁₋₆alkoxy optionally substituted with up to 9 halo, and C₁₋₆alkyl optionally substituted with up to 9 halo;

each R^{c}R^{d}N is separately selected, wherein R^{c} and R^{d} are each separately selected from the group consisting of hydrogen, alkoxyC(═O)—, C₁₋₆alkyl, C₁₋₆alkylC(═O)—, C₁₋₆alkylsulfonyl, arylalkylOC(═O)—, arylalkyl, arylalkylC(═O)—, arylC(═O)—, arylsulfonyl, heterocyclylalkyl, heterocyclylalkylC(═O)—, heterocyclylC(═O)—, (R^{e}R^{f}N)alkyl, (R^{e}R^{f}N)alkylC(═O)—, and (R^{e}R^{f}N)C(═O)—, wherein the alkyl part of arylalkyl, arylalkylC(═O)—, heterocyclylalkyl, and heterocyclylalkylC(═O)— are each optionally substituted with one R^{e}R^{f}N— group; and wherein the aryl part of arylalkyl, arylalkylC(═O)—, arylC(═O)—, and arylsulfonyl, and the heterocyclyl part of heterocyclylalkyl, heterocyclylalkylC(═O)—, and heterocyclylC(═O)— are each optionally substituted with up to three substituents each independently selected from the group consisting of cyano, halo, nitro, C₁₋₆alkoxy optionally substituted with up to 9 halo, and C₁₋₆alkyl optionally substituted with up to 9 halo;

each R^{e}R^{f}N is separately selected, wherein R^{e} and R^{f} are each separately selected from the group consisting of hydrogen, C₁₋₆alkyl, aryl, arylalkyl, cycloalkyl, (cyclolalkyl)alkyl, heterocyclyl, heterocyclylalkyl, (R^{x}R^{y}N)alkyl, and (R^{x}R^{y}N)C(═O)—;

each R^{x}R^{y}N is separately selected, wherein R^{x} and R^{y} are each separately selected from the group consisting of hydrogen, C₁₋₆alkylOC(═O)—, C₁₋₆alkyl, C₁₋₆alkylC(═O)—, aryl, arylalkyl, cycloalkyl, and heterocyclyl;

each C(R^{2a})₂ is separately selected, wherein each R^{2a} is separately selected from the group consisting of hydrogen, C₁₋₆alkyl optionally substituted with up to 9 halo, aryl(CH₂)ₙ—, and heteroaryl(CH₂)ₙ—, said aryl and heteroaryl each optionally substituted with cyano, halo, nitro, hydroxyl, C₁₋₆alkoxy optionally substituted with up to 9 halo, and C₁₋₆alkyl optionally substituted with up to 9 halo, or C(R^{2a})₂ is

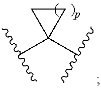

each R^{3a} is separately selected from the group consisting of hydrogen, and optionally substituted C₁₋₆alkyl;

each R^{3b} is separately selected from the group consisting of optionally substituted C₁₋₆alkyl, heteroaryl, —(CH₂)ₙC(═O)NR^{4a}R^{4b}, —(CH₂)ₙC(═O)OR^{5a}, and —(CH₂)ₙC(═O)R^{6a} said heteroaryl optionally substituted with cyano, halo, nitro, hydroxyl, C₁₋₆alkoxy optionally substituted with up to 9 halo, and C₁₋₆alkyl optionally substituted with up to 9 halo;

each R^{4a}R^{4b}N is separately selected, wherein R^{4a} and R^{4b} are each separately selected from the group consisting of hydrogen, optionally substituted C₁₋₆alkyl, and aryl(CH₂)ₙ—;

each R^{5a} is separately selected from the group consisting of C₁₋₆alkyl optionally substituted with up to five R² groups, and aryl(CH₂)ₙ—;

each R^{5a} is separately selected from the group consisting of optionally substituted C₁₋₆alkyl, and aryl(CH₂)ₙ—;

X¹ is (C(R²)₂)_{q},

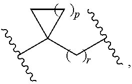

or X¹ is null;

Y¹ is selected from O (oxygen), S (sulfur), S(O), SO₂, NR², and C(R²)₂ with the proviso that when X¹ is null Y¹ is C(R²)₂;

X² is (C(R²)₂)_{q},

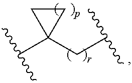

or X² is null;

Y² is selected from O (oxygen), S (sulfur), S(O), SO₂, NR², and C(R²)₂ with the proviso that when X² is null Y² is C(R²)₂;

each R² is separately selected, wherein R² is selected from the group consisting of hydrogen, deuterium, C₁₋₆alkoxy, C₁₋₆alkyl, aryl, halo, hydroxy, R^{a}R^{b}N—, and C₁₋₆alkyl optionally substituted with up to 9 halo, or optionally two R² and the carbons to which they are attached are together a fused three- to eight-membered carbocyclic ring optionally containing one or two heteroatoms each independently selected from O (oxygen), N (nitrogen), and S (sulfur); wherein the three- to eight-membered carbocyclic ring is optionally substituted with one or more substituents selected from the group consisting of deuterium, halo, hydroxyl, oxo, R^{x}R^{y}N, R^{x}R^{y}NC(═O), R^{x}R^{y}NC₁₋₆alkyl, heteroaryl, aryl, C₁₋₆alkyl optionally substituted with up to 9 halo, and C₁₋₆alkoxy optionally substituted with up to 9 halo, wherein at least one R² is deuterium or at least one of R^{2a}, R^{3a}, R^{4a}, R^{4b}, R^{5a}, and R^{6a} is a substituted C₁₋₆alkyl substituted with at least one substituent that is not alkyl or substituted aryl substituted with at least one substituent that is not alkyl;

each R^{a}R^{b}N is separately selected, wherein R^{a} and R^{b} are each separately selected from the group consisting of hydrogen, C₂₋₆alkenyl, and C₁₋₆alkyl;

each L¹ is separately selected from the group consisting of

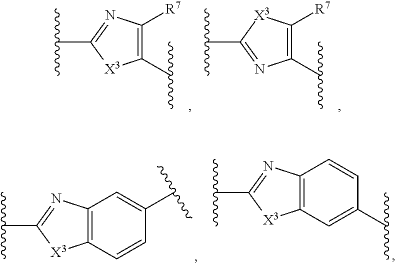

—C(═O)(CH₂)ₘOC(═O)—, —C(CF₃)₂NR^{2c}—, and

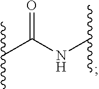

R^{2c} is selected from the group consisting of hydrogen, C₁₋₆alkyl, C₂₋₆alkenyl, C₂₋₆alkynyl, C₃₋₇cycloalkyl, aryl, arylalkyl, heteroaryl, heteroarylalkyl, heterocyclyl, and heterocyclylalkyl, said alkyl optionally substituted with R^{e}R^{f}N—, alkoxy, or C₁₋₆alkylS—;

each X³ is separately selected from the group consisting of NH, NC₁₋₆alkyl, O (oxygen), and S (sulfur);

each m separately is 1 or 2;

each n separately is 0, 1 or 2;

each p separately is 1, 2, 3 or 4;

each q separately is 1, 2, 3, 4 or 5;

each r separately is 0, 1, 2, 3, or 4; and

each R⁷ is separately selected from the group consisting of hydrogen, C₁₋₆alkylOC(═O)—, arylalkylOC(═O)—, —COOH, (R^{a}R^{b}N)C(═O)—, trialkylsilylalkylOalkyl, and C₁₋₆alkyl optionally substituted with up to 9 halo.

In some embodiments of Formula IX, each R¹ is R^{1a}C(═O)—. In some embodiments of Formula IX, each R^{1a} is —CHR^{2a}NHR^{3b}. In some embodiments of Formula IX, each R^{2a} is C₁₋₆alkyl; each R^{3b} is —C(═O)OR⁵; and each R⁵ is C₁₋₆alkyl.

In some embodiments of Formula IX, each at least one R^{1a} is —C(R^{2a})₂NR^{3a}R^{3b} and within said at least one R^{1a}, at least one R^{2a} is a substituted C₁₋₆alkyl substituted with at least one substituent that is not alkyl or substituted aryl substituted with at least one substituent that is not alkyl. In some embodiments of Formula IX, at least one R^{1a} is —C(R^{2a})₂NR^{3a}R^{3b} wherein R^{3b} is —(CH₂)ₙC(═O)OR^{5a} and R^{5a} is a substituted C₁₋₆alkyl substituted with at least one substituent that is not alkyl or substituted aryl substituted with at least one substituent that is not alkyl. In some embodiments of Formula IX, at least one of R^{2a}, R^{3a}, R^{4a}, R^{4b}, R^{5a}, and R^{6a} is C₁₋₆alkyl substituted with up to 9 halogen or aryl substituted with up to 9 halogen. In some embodiments of Formula IX, each R² is separately selected from the group consisting of hydrogen, C₁₋₆alkoxy, aryl, halo, hydroxy, R^{a}R^{b}N—, and C₁₋₆alkyl optionally substituted with up to 9 halo. In some embodiments of Formula IX, at least one of R^{2a}, R^{3a}, R^{4a}, and R^{6a} is C₁₋₆alkyl substituted with up to 9 halogen or aryl substituted with up to 9 halogen.

In some embodiments, the compound of Formula IX has the structure of Formula IXa:

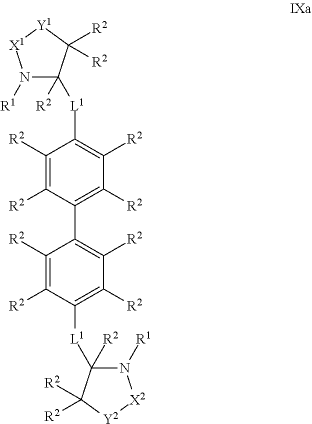

or a pharmaceutically acceptable salt thereof, wherein R⁶ is C₁₋₆alkyl optionally substituted with up to 9 halo. In some embodiments, R⁶ is methyl.

Some embodiments include a compound having the structure of Formula X:

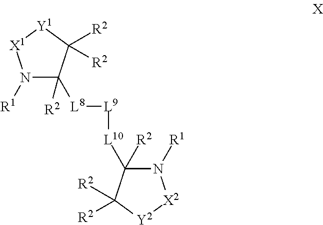

or a pharmaceutically acceptable salt thereof,

wherein:

each R¹ is separately selected from the group consisting of hydrogen, R^{1a}C(═O)— and R^{1a}C(═S)—;

each R^{1a} is separately selected from the group consisting of —C(R^{2a})₂NR^{3a}R^{3b}, alkoxyalkyl, C₁₋₆alkylOC(═O)—, C₁₋₆alkylOC(═O)C₁₋₆alkyl, C₁₋₆alkylC(═O)C₁₋₆alkyl, aryl, aryl(CH₂)ₙ—, aryl(CH₂)ₙO—, aryl(CH═CH)ₘ—, arylalkylO—, arylalkyl, arylOalkyl, cycloalkyl, (cycloalkyl)(CH═CH)ₘ—, (cycloalkyl)alkyl, cycloalkylOalkyl, heterocyclyl, heterocyclyl(CH═CH)ₘ—, heterocyclylalkoxy, heterocyclylalkyl, heterocyclylOalkyl, hydroxyalkyl, R^{c}R^{d}N—, R^{c}R^{d}N(CH₂)ₙ—, (R^{c}R^{d}N)(CH═CH)ₘ—, (R^{c}R^{d}N)alkyl, (R^{c}R^{d}N)C(═O)—, C₁₋₆alkoxy optionally substituted with up to 9 halo, and C₁₋₆alkyl optionally substituted with up to 9 halo, said aryl and heteroaryl each optionally substituted with cyano, halo, nitro, hydroxyl, C₁₋₆alkoxy optionally substituted with up to 9 halo, and C₁₋₆alkyl optionally substituted with up to 9 halo;

each R^{c}R^{d}N is separately selected, wherein R^{c} and R^{d} are each separately selected from hydrogen, alkoxyC(═O)—, C₁₋₆alkyl, C₁₋₆alkylC(═O)—, C₁₋₆alkylsulfonyl, arylalkylOC(═O)—, arylalkyl, arylalkylC(═O)—, arylC(═O)—, arylsulfonyl, heterocyclylalkyl, heterocyclylalkylC(═O)—, heterocyclylC(═O)—, (R^{e}R^{f}N)alkyl, (R^{e}R^{f}N)alkylC(═O)—, and (R^{e}R^{f}N)C(═O)—, wherein the alkyl part of arylalkyl, arylalkylC(═O)—, heterocyclylalkyl, and heterocyclylalkylC(═O)— are each optionally substituted with one R^{e}R^{f}N— group; and wherein the aryl part of arylalkyl, arylalkylC(═O)—, arylC(═O)—, and arylsulfonyl, and the heterocyclyl part of heterocyclylalkyl, heterocyclylalkylC(═O)—, and heterocyclylC(═O)— are each optionally substituted with up to three substituents each independently selected from the group consisting of cyano, halo, nitro, C₁₋₆alkoxy optionally substituted with up to 9 halo, and C₁₋₆alkyl optionally substituted with up to 9 halo;

each R^{e}R^{f}N is separately selected, wherein R^{e} and R^{f} are each separately selected from hydrogen, C₁₋₆alkyl, aryl, arylalkyl, cycloalkyl, (cyclolalkyl)alkyl, heterocyclyl, heterocyclylalkyl, (R^{x}R^{y}N)alkyl, and (R^{x}R^{y}N)C(═O)—;

each R^{x}R^{y}N is separately selected, wherein R^{x} and R^{y} are each separately selected from hydrogen, C₁₋₆alkylOC(═O)—, C₁₋₆alkyl, C₁₋₆alkylC(═O)—, aryl, arylalkyl, cycloalkyl, and heterocyclyl;

each C(R^{2a})₂ is separately selected, wherein each R^{2a} is separately selected from the group consisting of hydrogen, optionally substituted C₁₋₆alkyl, aryl(CH₂)ₙ—, and heteroaryl(CH₂)ₙ—, said aryl and heteroaryl each optionally substituted with cyano, halo, nitro, hydroxyl, C₁₋₆alkoxy optionally substituted with up to 9 halo, and C₁₋₆alkyl optionally substituted with up to 9 halo, or C(R^{2a})₂ is

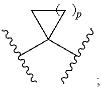

each R^{3a} is separately selected from the group consisting of hydrogen, and optionally substituted C₁₋₆alkyl;

each R^{3b} is separately selected from the group consisting of optionally substituted C₁₋₆alkyl, heteroaryl, —(CH₂)ₙC(═O)NR^{4a}R^{4b}, —(CH₂)ₙC(═O)OR^{5a}, and —(CH₂)ₙC(═O)R^{6a} said heteroaryl optionally substituted with cyano, halo, nitro, hydroxyl, C₁₋₆alkoxy optionally substituted with up to 9 halo, and C₁₋₆alkyl optionally substituted with up to 9 halo;

each R^{4a}R^{4b}N is separately selected, wherein R^{4a} and R^{4b} are each separately selected from the group consisting of hydrogen, optionally substituted C₁₋₆alkyl, and aryl(CH₂)ₙ—;

each R^{5a} is separately selected from the group consisting of optionally substituted C₁₋₆alkyl and aryl(CH₂)ₙ—;

each R^{6a} is separately selected from the group consisting of optionally substituted C₁₋₆alkyl, and aryl(CH₂)ₙ—;

X¹ is (C(R²)₂)_{q},

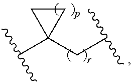

or X¹ is null;

Y¹ is selected from O (oxygen), S (sulfur), S(O), SO₂, NR², and C(R²)₂ with the proviso that when X¹ is null Y¹ is C(R²)₂;

X² is (C(R²)₂)_{q},

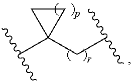

or X² is null;

Y² is selected from O (oxygen), S (sulfur), S(O), SO₂, NR², and C(R²)₂ with the proviso that when X² is null Y² is C(R²)₂;

each R² is separately selected, wherein R² is selected from the group consisting of hydrogen, C₁₋₆alkoxy, aryl, halo, hydroxy, R^{a}R^{b}N—, and C₁₋₆alkyl optionally substituted with up to 9 halo, or optionally two R² and the carbons to which they are attached are together a fused three- to eight-membered carbocyclic ring optionally containing one or two heteroatoms each independently selected from O (oxygen), N (nitrogen), and S (sulfur); wherein the three- to eight-membered carbocyclic ring is optionally substituted with one or more substituents selected from the group consisting of halo, hydroxyl, oxo, R^{x}R^{y}N, R^{x}R^{y}NC(═O), R^{x}R^{y}NC₁₋₆alkyl, heteroaryl, aryl, C₁₋₆alkyl optionally substituted with up to 9 halo, and C₁₋₆alkoxy optionally substituted with up to 9 halo;

each R^{a}R^{b}N is separately selected, wherein R^{a} and R^{b} are each separately selected from the group consisting of hydrogen, C₂₋₆alkenyl, and C₁₋₆alkyl;

L⁸ is selected from the group consisting of

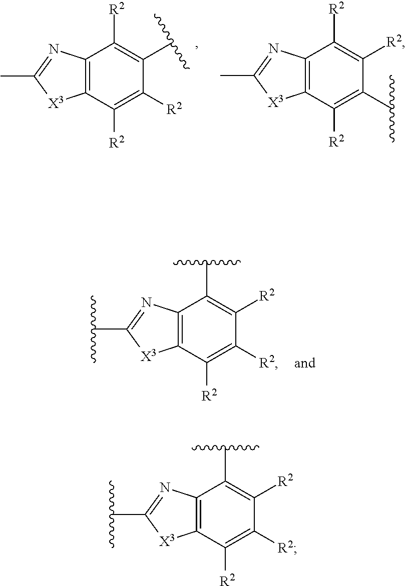

L⁹ is selected from the group consisting of

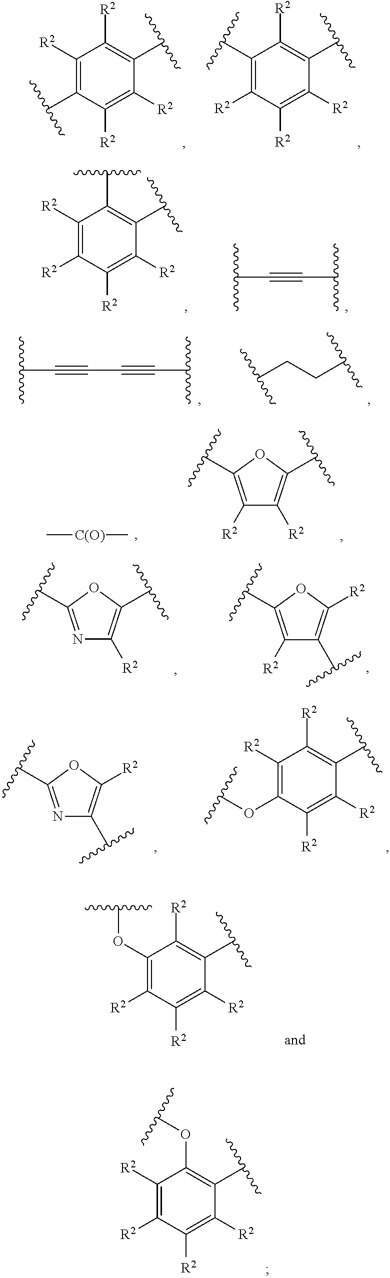

L¹⁰ is selected from the group consisting of

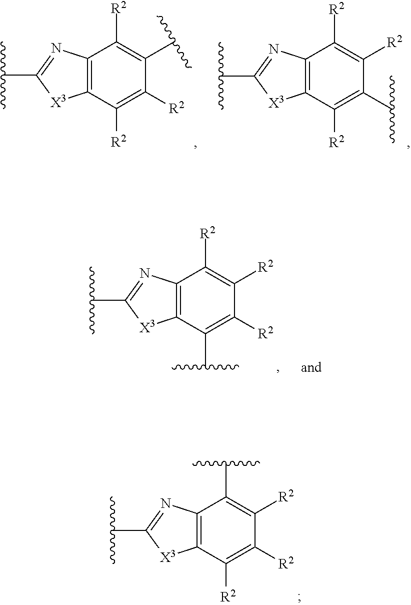

each X³ is separately selected from the group consisting of NH, NC₁₋₆alkyl, O (oxygen), and S (sulfur);

each m separately is 1 or 2;

each n separately is 0, 1 or 2;

each p separately is 1, 2, 3 or 4;

each q separately is 1, 2, 3, 4 or 5; and

each r separately is 0, 1, 2, 3, or 4,

wherein at least one of R^{2a}, R^{3a}, R^{4a}, R^{4b}, R^{5a}, and R^{6a} is a substituted C₁₋₆alkyl.

In some embodiments of Formula X, each R¹ is R^{1a}C(═O)—. In some embodiments of Formula X, each R^{1a} is —CHR^{2a}NHR^{3b}. In some embodiments of Formula X, each R^{2a} is C₁₋₆alkyl; each R^{3b} is —C(═O)OR⁵; and each R⁵ is C₁₋₆alkyl.

In some embodiments of Formula X, at least one R^{1a} is —C(R^{2a})₂NR^{3a}R^{3b} and within said at least one R^{1a}, at least one R^{2a} is a substituted C₁₋₆alkyl. In some embodiments of Formula X, at least one R^{1a} is —C(R^{2a})₂NR^{3a}R^{3b} wherein R^{3b} is —(CH₂)ₙC(═O)OR^{5a} and R^{5a} is a substituted C₁₋₆alkyl. In some embodiments of Formula X, at least one of R^{2a}, R^{3a}, R^{4a}, R^{4b}, R^{5a}, and R^{6a} is C₁₋₆alkyl substituted with up to 9 halogen or aryl substituted with up to 9 halogen. In some embodiments of Formula X, each R² is separately selected from the group consisting of hydrogen, C₁₋₆alkoxy, aryl, halo, hydroxy, R^{a}R^{b}N—, and C₁₋₆alkyl optionally substituted with up to 9 halo. In some embodiments of Formula X, at least one of R^{2a}, R^{3a}, R^{4a}, R^{4b}, and R^{6a} is C₁₋₆alkyl substituted with up to 9 halogen or aryl substituted with up to 9 halogen. In some embodiments of Formula X, L⁹ is selected from the group consisting of:

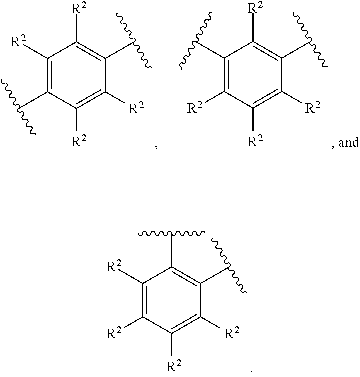

In some embodiments, the compound of Formula X has the structure of Formula Xa:

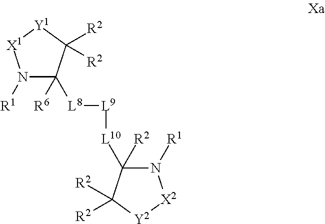

or a pharmaceutically acceptable salt thereof, wherein R⁶ is C₁₋₆alkyl optionally substituted with up to 9 halo. In some embodiments, R⁶ is methyl.

Some embodiments include a compound having the structure of Formula XI:

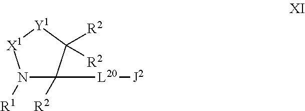

or a pharmaceutically acceptable salt thereof,

wherein:

each R¹ is separately selected from the group consisting of hydrogen, R^{1a}C(═O)— and R^{1a}C(═S)—;

R^{1a} is selected from the group consisting of —C(R^{2a})₂NR^{3a}R^{3b}, alkoxyalkyl, C₁₋₆alkylOC(═O)—, C₁₋₆alkylOC(═O)C₁₋₆alkyl, C₁₋₆alkylC(═O)C₁₋₆alkyl, aryl, aryl(CH₂)ₙ—, aryl(CH₂)ₙO—, aryl(CH═CH)ₘ—, arylalkylO—, arylalkyl, arylOalkyl, cycloalkyl, (cycloalkyl)(CH═CH)ₘ—, (cycloalkyl)alkyl, cycloalkylOalkyl, heterocyclyl, heterocyclyl(CH═CH)ₘ—, heterocyclylalkoxy, heterocyclylalkyl, heterocyclylOalkyl, hydroxyalkyl, R^{c}R^{d}N—, R^{c}R^{d}N(CH₂)ₙ—, (R^{c}R^{d}N)(CH═CH)ₘ—, (R^{c}R^{d}N)alkyl, (R^{c}R^{d}N)C(═O)—, C₁₋₆alkoxy optionally substituted with up to 9 halo, and C₁₋₆alkyl optionally substituted with up to 9 halo, said aryl and heteroaryl each optionally substituted with cyano, halo, nitro, hydroxyl, C₁₋₆alkoxy optionally substituted with up to 9 halo, and C₁₋₆alkyl optionally substituted with up to 9 halo;

R^{c} and R^{d} are each separately selected from the group consisting of hydrogen, alkoxyC(═O)—, C₁₋₆alkyl, C₁₋₆alkylC(═O)—, C₁₋₆alkylsulfonyl, arylalkylOC(═O)—, arylalkyl, arylalkylC(═O)—, arylC(═O)—, arylsulfonyl, heterocyclylalkyl, heterocyclylalkylC(═O)—, heterocyclylC(═O)—, (R^{e}R^{f}N)alkyl, (R^{e}R^{f}N)alkylC(═O)—, and (R^{e}R^{f}N)C(═O)—, wherein the alkyl part of arylalkyl, arylalkylC(═O)—, heterocyclylalkyl, and heterocyclylalkylC(═O)— are each optionally substituted with one R^{e}R^{f}N— group; and wherein the aryl part of arylalkyl, arylalkylC(═O)—, arylC(═O)—, and arylsulfonyl, and the heterocyclyl part of heterocyclylalkyl, heterocyclylalkylC(═O)—, and heterocyclylC(═O)— are each optionally substituted with up to three substituents each independently selected from the group consisting of cyano, halo, nitro, C₁₋₆alkoxy optionally substituted with up to 9 halo, and C₁₋₆alkyl optionally substituted with up to 9 halo;

each R^{e}R^{f}N is separately selected, wherein R^{e} and R^{f} are each separately selected from the group consisting of hydrogen, C₁₋₆alkyl, aryl, arylalkyl, cycloalkyl, (cyclolalkyl)alkyl, heterocyclyl, heterocyclylalkyl, (R^{x}R^{y}N)alkyl, and (R^{x}R^{y}N)C(═O);

each R^{x}R^{y}N is separately selected, wherein R^{x} and R^{y} are each separately selected from hydrogen, C₁₋₆alkylOC(═O)—, C₁₋₆alkyl, C₁₋₆alkylC(═O)—, aryl, arylalkyl, cycloalkyl, and heterocyclyl;

R^{2a} is selected from the group consisting of hydrogen, optionally substituted C₁₋₆alkyl, aryl(CH₂)ₙ—, and heteroaryl(CH₂)ₙ—, said aryl and heteroaryl each optionally substituted with cyano, halo, nitro, hydroxyl, C₁₋₆alkoxy optionally substituted with up to 9 halo, and C₁₋₆alkyl optionally substituted with up to 9 halo, or C(R^{2a})₂ is

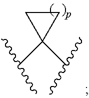

R^{3a} is selected from the group consisting of hydrogen, and optionally substituted C₁₋₆alkyl;

R^{3b} is selected from the group consisting of optionally substituted C₁₋₆alkyl, heteroaryl, —(CH₂)ₙC(═O)NR^{4a}R^{4b}, —(CH₂)ₙC(═O)OR^{5a}, and —(CH₂)ₙC(═O)R^{6a} said heteroaryl optionally substituted with one or more substituents selected from the group consisting of cyano, halo, nitro, hydroxyl, C₁₋₆alkoxy optionally substituted with up to 9 halo, and C₁₋₆alkyl optionally substituted with up to 9 halo;

R^{4a} and R^{4b} are each separately selected from the group consisting of hydrogen, optionally substituted C₁₋₆alkyl, and aryl(CH₂)ₙ—;

each R^{5a} is separately selected from the group consisting of optionally substituted C₁₋₆alkyl and aryl(CH₂)ₙ—;

each R^{6a} is separately selected from the group consisting of optionally substituted C₁₋₆alkyl, and aryl(CH₂)ₙ—;

X¹ is (C(R²)₂)_{q},

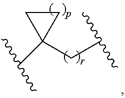

or X¹ is null;

Y¹ is selected from O (oxygen), S (sulfur), S(O), SO₂, NR², and C(R²)₂ with the proviso that when X¹ is null Y¹ is C(R²)₂;

each R² is separately selected, wherein R² is selected from the group consisting of hydrogen, C₁₋₆alkoxy, aryl, halo, hydroxy, R^{a}R^{b}N—, and C₁₋₆alkyl optionally substituted with up to 9 halo, or optionally two R² and the carbons to which they are attached are together a fused three- to eight-membered carbocyclic ring optionally containing one or two heteroatoms each independently selected from O (oxygen), N (nitrogen), and S (sulfur); wherein the three- to eight-membered carbocyclic ring is optionally substituted with one or more substituents selected from the group consisting of halo, hydroxyl, oxo, R^{x}R^{y}N, R^{x}R^{y}NC(═O), R^{x}R^{y}NC₁₋₆alkyl, heteroaryl, aryl, C₁₋₆alkyl optionally substituted with up to 9 halo, and C₁₋₆alkoxy optionally substituted with up to 9 halo, or optionally two geminal R² and the carbon to which they are attached are together carbonyl, or optionally two geminal R² and the carbon to which they are attached are together a three- to six-membered carbocyclic ring optionally substituted with up to two C₁₋₆alkyl groups;

each R^{a}R^{b}N is separately selected, wherein R^{a} and R^{b} are each separately selected from the group consisting of hydrogen, C₂₋₆alkenyl, and C₁₋₆alkyl;

L²⁰ is selected from the group consisting of Q¹-Q², Q³-Q⁴,

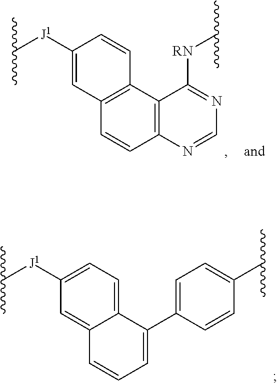

Q¹ is selected from the group consisting of J⁵, J⁴-J⁵, J¹-J⁵-J¹⁰, J¹-J⁵-J³,

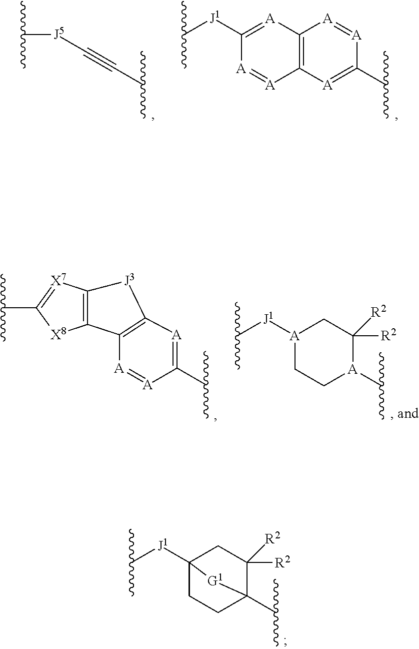

Q² is

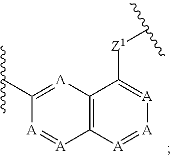

Q³ is selected from the group consisting of, J⁵, J¹-J⁵, J¹-J⁵-J¹⁰, J¹-J⁵-J³,

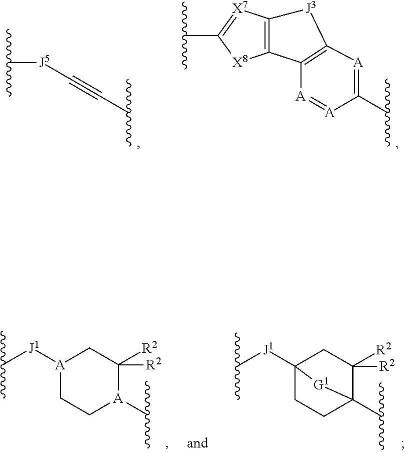

Q⁴ is selected from the group consisting of

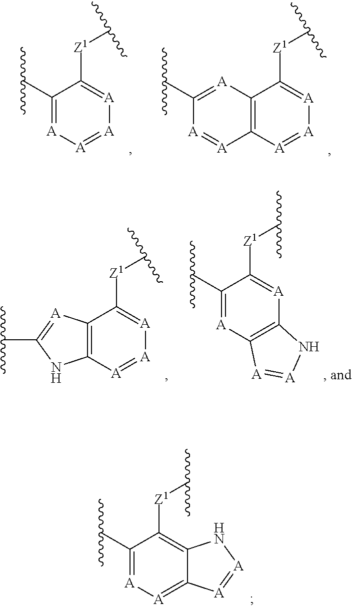

Z¹ is selected from the group consisting of O (oxygen), S (sulfur), NR, and C(R²)₂;

each A is separately selected from the group consisting of CR³ and N (nitrogen);

each R³ is separately selected from the group consisting of hydrogen, C₁₋₆alkoxy, C₁₋₆alkylOC₁₋₆alkyl, C₁₋₆alkylOC(═O)—, arylalkylOC(═O)—, —COOH, halo, hydroxy, R^{a}R^{b}N—, (R^{a}R^{b}N)alkyl, (R^{a}R^{b}N)C(═O)—, and C₁₋₆alkyl optionally substituted with up to 9 halo and up to 5 hydroxy;

G¹ is —CH₂— or —CH₂CH₂—;

J¹ is

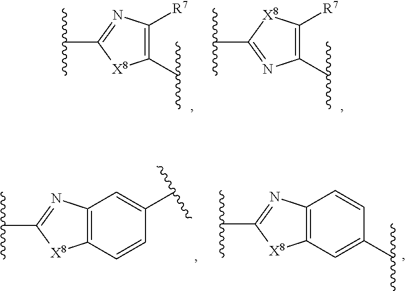

### —C(CF₃)₂NR^{2c}—, or

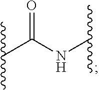

R^{2c} is selected from the group consisting of hydrogen, C₁₋₆alkyl, C₂₋₆alkenyl, C₂₋₆alkynyl, C₃₋₇cycloalkyl, aryl, arylalkyl, heteroaryl, heteroarylalkyl, heterocyclyl, and heterocyclylalkyl, said alkyl optionally substituted with R^{e}R^{f}N—, alkoxy, or C₁₋₆alkylS—;

J² is aryl, heteroaryl, heterocyclyl, or polycyclic hydrocarbon, each optionally substituted one or more substituents independently selected from the group consisting of halo, hydroxyl, R^{x}R^{y}N, R^{x}R^{y}NC(═O), R^{x}R^{y}NC₁₋₆alkyl, heteroaryl, aryl, C₁₋₆alkyl optionally substituted with up to 9 halo, and C₁₋₆alkoxy optionally substituted with up to 9 halo, said substituent aryl and heteroaryl are each optionally substituted with one or more R¹⁴;

each R¹⁴ is separately selected from the group consisting of hydroxy, C₁₋₆alkoxy optionally substituted with up to 9 fluoro, C₁₋₆alkylOC₁₋₆alkyl, C₁₋₆alkylOC(═O)—, arylalkylOC(═O)—, —COOH, halo, and C₁₋₆alkyl optionally substituted with up to 9 halo;

J³ is C₂₋₄ alkyl, NH, O (oxygen), —NHC(O)—, S (sulfur), —(CH₂)ₙX⁸(CH₂)ₘ—, or —X⁷═X⁷;

J⁴ is

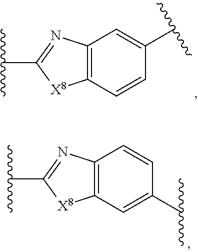

### —C(CF₃)₂NR^{2c}—, or

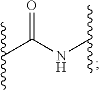

J⁵ is aryl, heteroaryl, heterocyclyl, or polycyclic hydrocarbon, each optionally substituted one or more R³;

each X⁷ is separately selected from the group consisting of N (nitrogen), and CR²;

each X⁸ is separately selected from the group consisting of NH, NC₁₋₆alkyl, O (oxygen), and S (sulfur);

J¹⁰ is —C(R²)₂—, —NR—, oxygen (O), or sulfur (S);

each m separately is 1 or 2;

each n separately is 0, 1 or 2;

each p separately is 1, 2, 3 or 4;

each q separately is 1, 2, 3, 4 or 5;

each r separately is 0, 1, 2, 3, or 4;

each R is separately selected from the group consisting of hydrogen, C₁₋₆alkylOC(═O)—, arylalkylOC(═O)—, (R^{a}R^{b}N)C(═O)—, and C₁₋₆alkyl optionally substituted with up to 9 halo; and

R⁷ is selected from the group consisting of hydrogen, halo, C₁₋₆alkylOC(═O)—, arylalkylOC(═O)—, —COOH, (R^{a}R^{b}N)C(═O)—, trialkylsilylalkylOalkyl, and C₁₋₆alkyl optionally substituted with up to 9 halo.

In some embodiments of Formula XI, L²⁰ is selected from the group consisting of

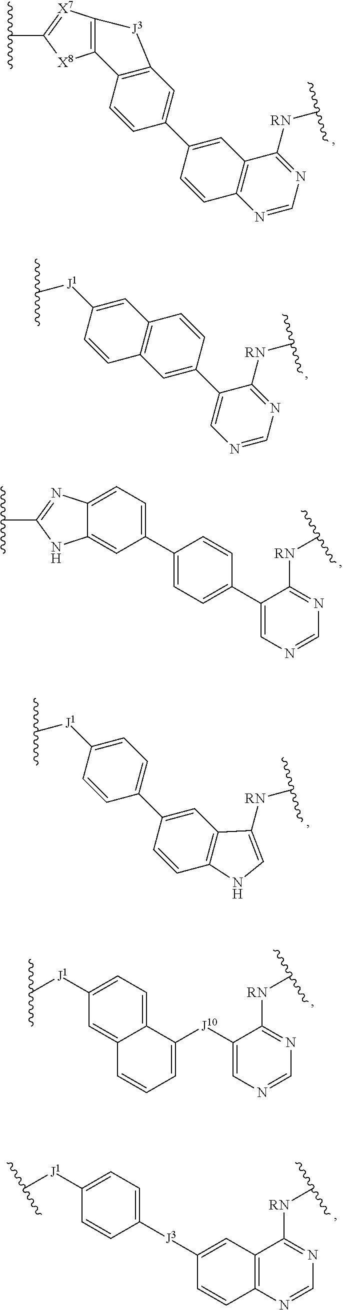

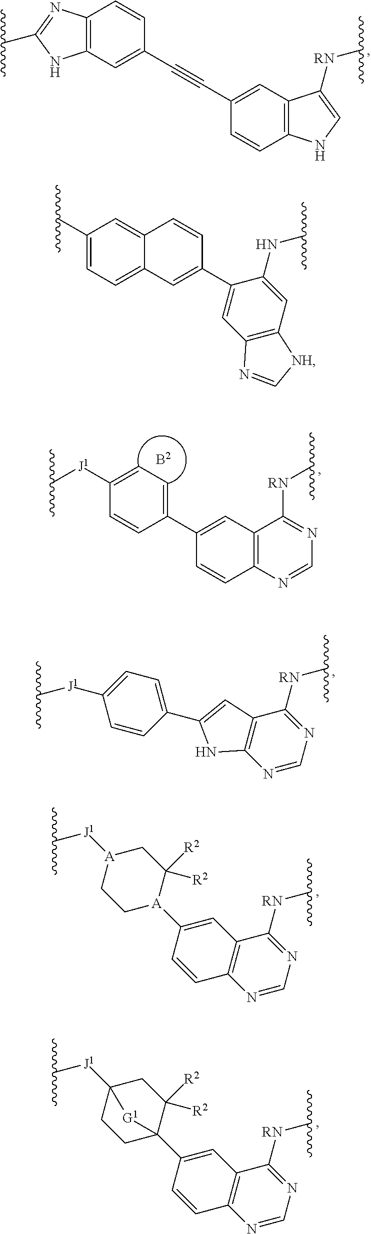

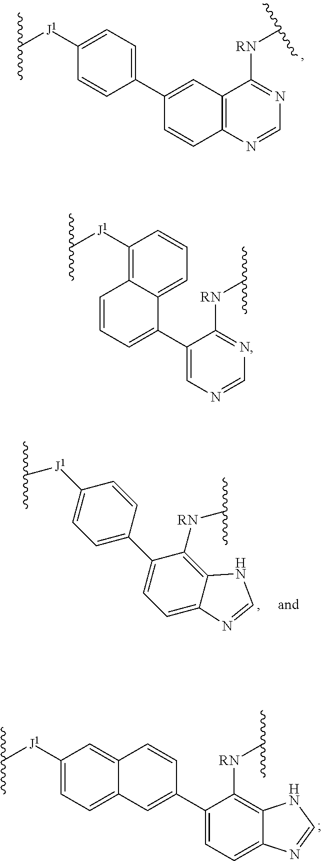

B² is an aliphatic ring optionally including O (oxygen), S (sulfur), or NH, or an aromatic ring optionally including N (nitrogen), said aliphatic or aromatic ring in the definition of B₂ is optionally substituted with one or more R^{g}; and

each R^{g} is separately selected from the group consisting of halo, hydroxy, C₁₋₆alkoxy optionally substituted with up to 9 fluoro, and C₁₋₆alkyl optionally substituted with up to 9 halo.

In some embodiments of Formula XI, Q¹ is selected from the group consisting of

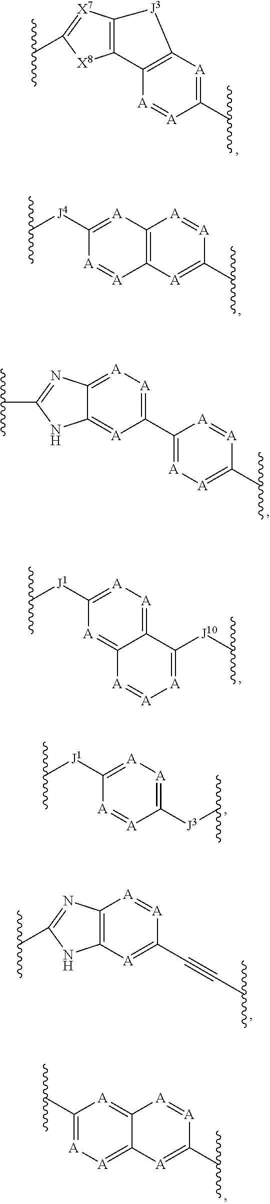

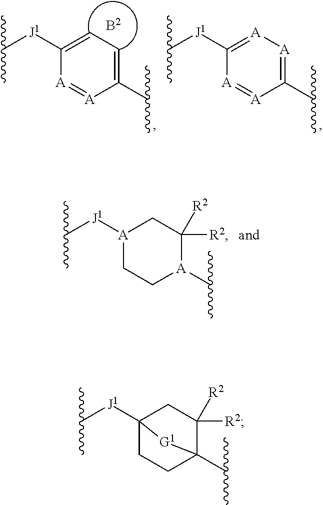

B² is an aliphatic ring optionally including O (oxygen), S (sulfur), or NH, or an aromatic ring optionally including N (nitrogen), said aliphatic or aromatic ring in the definition of B² is optionally substituted with one or more R^{g}; and

each R^{g} is separately selected from the group consisting of halo, hydroxy, C₁₋₆alkoxy optionally substituted with up to 9 fluoro, and C₁₋₆alkyl optionally substituted with up to 9 halo.

In some embodiments of Formula XI, Q³ is selected from the group consisting of

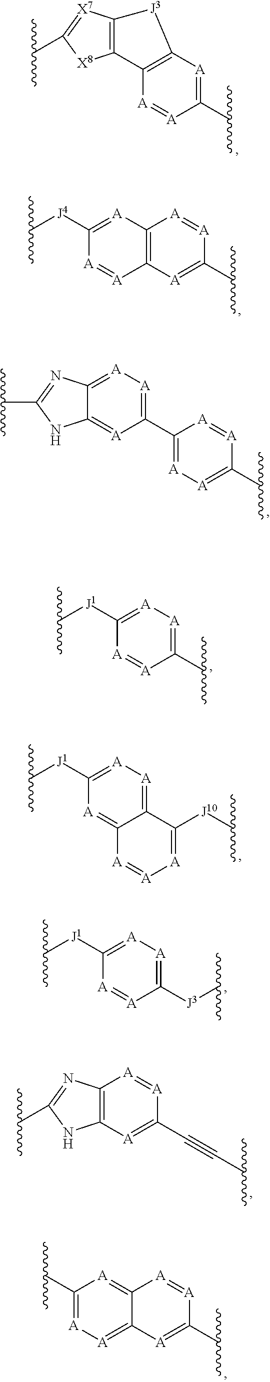

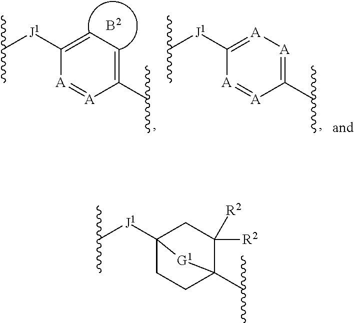

B² is an aliphatic ring optionally including O (oxygen), S (sulfur), or NH, or an aromatic ring optionally including N (nitrogen), said aliphatic or aromatic ring in the definition of B² is optionally substituted with one or more R^{g}; and

each R^{g} is separately selected from the group consisting of halo, hydroxy, C₁₋₆alkoxy optionally substituted with up to 9 fluoro, and C₁₋₆alkyl optionally substituted with up to 9 halo.

In some embodiments, the compound Formula XI has the structure

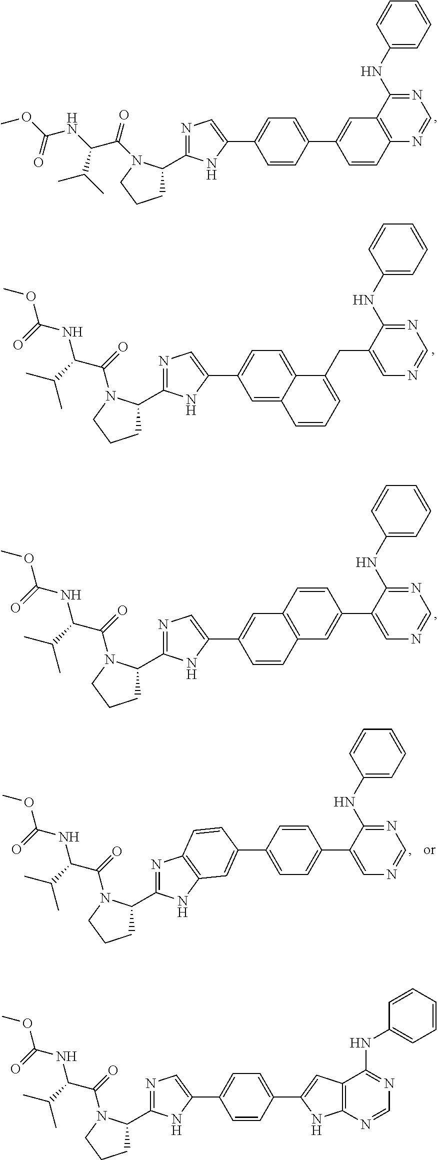

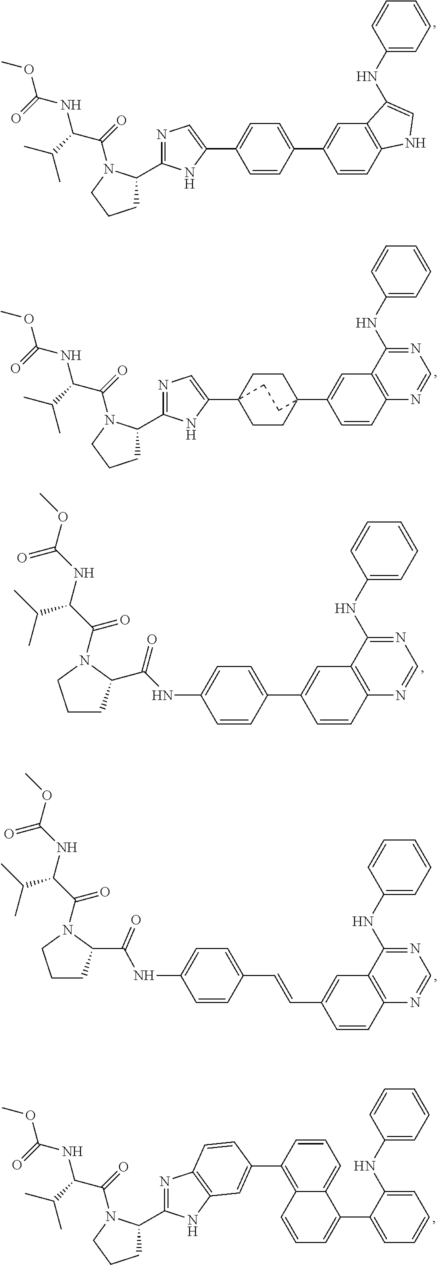

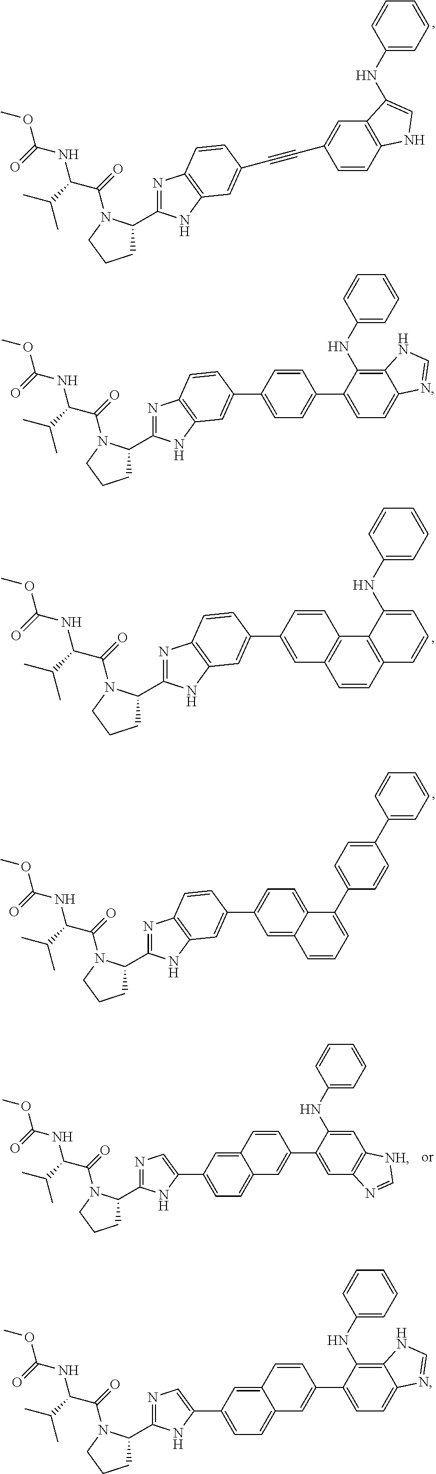

or a pharmaceutically acceptable salt thereof.

Some embodiments include a compound having the structure of Formula XII:

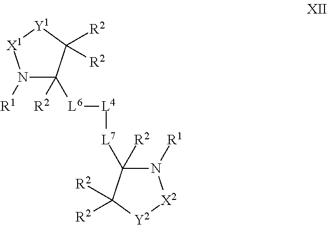

or a pharmaceutically acceptable salt thereof,

wherein:

each R¹ is separately selected from the group consisting of hydrogen, R^{1a}C(═O)— and R^{1a}C(═S)—;

each R^{1a} is separately selected from the group consisting of —C(R^{2a})₂NR^{3a}R^{3b}, alkoxyalkyl, C₁₋₆alkylOC(═O)—, C₁₋₆alkylOC(═O)C₁₋₆alkyl, C₁₋₆alkylC(═O)C₁₋₆alkyl, aryl, aryl(CH₂)ₙ—, aryl(CH₂)ₙO—, aryl(CH═CH)ₘ—, arylalkylO—, arylalkyl, arylOalkyl, cycloalkyl, (cycloalkyl)(CH═CH)ₘ—, (cycloalkyl)alkyl, cycloalkylOalkyl, heterocyclyl, heterocyclyl(CH═CH)ₘ—, heterocyclylalkoxy, heterocyclylalkyl, heterocyclylOalkyl, hydroxyalkyl, R^{c}R^{d}N—, R^{c}R^{d}N(CH₂)ₙ—, (R^{c}R^{d}N)(CH═CH)ₘ—, (R^{c}R^{d}N)alkyl, (R^{c}R^{d}N)C(═O)—, C₁₋₆alkoxy optionally substituted with up to 9 halo, and C₁₋₆alkyl optionally substituted with up to 9 halo, said aryl and heteroaryl each optionally substituted with cyano, halo, nitro, hydroxyl, C₁₋₆alkoxy optionally substituted with up to 9 halo, and C₁₋₆alkyl optionally substituted with up to 9 halo;

each R^{c}R^{d}N is separately selected, wherein R^{c} and R^{d} are each separately selected from hydrogen, alkoxyC(═O)—, C₁₋₆alkyl, C₁₋₆alkylC(═O)—, C₁₋₆alkylsulfonyl, arylalkylOC(═O)—, arylalkyl, arylalkylC(═O)—, arylC(═O)—, arylsulfonyl, heterocyclylalkyl, heterocyclylalkylC(═O)—, heterocyclylC(═O)—, (R^{e}R^{f}N)alkyl, (R^{e}R^{f}N)alkylC(═O)—, and (R^{e}R^{f}N)C(═O)—, wherein the alkyl part of arylalkyl, arylalkylC(═O)—, heterocyclylalkyl, and heterocyclylalkylC(═O)— are each optionally substituted with one R^{e}R^{f}N— group; and wherein the aryl part of arylalkyl, arylalkylC(═O)—, arylC(═O)—, and arylsulfonyl, and the heterocyclyl part of heterocyclylalkyl, heterocyclylalkylC(═O)—, and heterocyclylC(═O)— are each optionally substituted with up to three substituents each independently selected from the group consisting of cyano, halo, nitro, C₁₋₆alkoxy optionally substituted with up to 9 halo, and C₁₋₆alkyl optionally substituted with up to 9 halo;

each R^{e}R^{f}N is separately selected, wherein R^{e} and R^{f} are each separately selected from hydrogen, C₁₋₆alkyl, aryl, arylalkyl, cycloalkyl, (cyclolalkyl)alkyl, heterocyclyl, heterocyclylalkyl, (R^{x}R^{y}N)alkyl, and (R^{x}R^{y}N)C(═O)—;

each R^{x}R^{y}N is separately selected, wherein R^{x} and R^{y} are each separately selected from hydrogen, C₁₋₆alkylOC(═O)—, C₁₋₆alkyl, C₁₋₆alkylC(═O)—, aryl, arylalkyl, cycloalkyl, and heterocyclyl;

each C(R^{2a})₂ is separately selected, wherein each R^{2a} is separately selected from the group consisting of hydrogen, C₁₋₆alkyl optionally substituted with up to 9 halo, aryl(CH₂)ₙ—, and heteroaryl(CH₂)ₙ—, said aryl and heteroaryl each optionally substituted with cyano, halo, nitro, hydroxyl, C₁₋₆alkoxy optionally substituted with up to 9 halo, and C₁₋₆alkyl optionally substituted with up to 9 halo, or C(R^{2a})₂ is

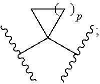

each R^{3a} is separately selected from the group consisting of hydrogen, and optionally substituted C₁₋₆alkyl;

each R^{3b} is separately selected from the group consisting of optionally substituted C₁₋₆alkyl, heteroaryl, —(CH₂)ₙC(═O)NR^{4a}R^{4b}, —(CH₂)ₙC(═O)OR^{5a}, and —(CH₂)ₙC(═O)R^{6a} said heteroaryl optionally substituted with cyano, halo, nitro, hydroxyl, C₁₋₆alkoxy optionally substituted with up to 9 halo, and C₁₋₆alkyl optionally substituted with up to 9 halo;

each R^{4a}R^{4b}N is separately selected, wherein R^{4a} and R^{4b} are each separately selected from the group consisting of hydrogen, optionally substituted C₁₋₆alkyl, and aryl(CH₂)ₙ—;

each R^{5a} is separately selected from the group consisting of optionally substituted C₁₋₆alkyl, and aryl(CH₂)ₙ—;

each R^{6a} is separately selected from the group consisting of optionally substituted C₁₋₆alkyl, and aryl(CH₂)ₙ—;

X¹ is (C(R²)₂)_{q},

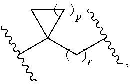

or X¹ is null;

Y¹ is selected from O (oxygen), S (sulfur), S(O), SO₂, NR², and C(R²)₂ with the proviso that when X¹ is null Y¹ is C(R²)₂;

X² is (C(R²)₂)_{q},

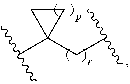

or X² is null;

Y² is selected from O (oxygen), S (sulfur), S(O), SO₂, NR², and C(R²)₂ with the proviso that when X² is null Y² is C(R²)₂;

each R² is separately selected, wherein R² is selected from the group consisting of hydrogen, C₁₋₆alkoxy, C₁₋₆alkyl, aryl, halo, hydroxy, R^{a}R^{b}N—, and C₁₋₆alkyl optionally substituted with up to 9 halo, or optionally two vicinal R² and the carbons to which they are attached are together a fused three- to six-membered carbocyclic ring optionally substituted with up to two C₁₋₆alkyl groups;

each R^{a}R^{b}N is separately selected, wherein R^{a} and R^{b} are each separately selected from the group consisting of hydrogen, C₂₋₆alkenyl, and C₁₋₆alkyl;

each A is separately selected from the group consisting of CR³ and N (nitrogen);

L⁴ is selected from the group consisting of -(J²)ₛ-(L⁵)ₛ-(J²)ₛ-(L⁵)ₛ-J²-,

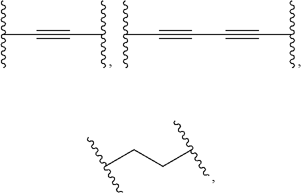

—C(═O)—, O (oxygen), —OC(R²)₂—,

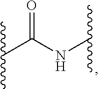

### —C(CF₃)₂NR^{2c}—, NH, and —(CH═CH)—;

J² is aryl, heteroaryl, heterocyclyl, cycloalkyl, cycloalkenyl, or polycyclic hydrocarbon, each optionally substituted with one or more R¹⁵;

each R¹⁴ is separately selected from the group consisting of hydroxy, C₁₋₆alkoxy optionally substituted with up to 9 fluoro, C₁₋₆alkylOC₁₋₆alkyl, C₁₋₆alkylOC(═O)—, arylalkylOC(═O)—, —COOH, halo, and C₁₋₆alkyl optionally substituted with up to 9 halo;

each R¹⁵ is separately selected from the group consisting of halo, hydroxy, C₁₋₆alkylOC₁₋₆alkyl, C₁₋₆alkylOC(═O)—, arylalkylOC(═O)—, —COOH, R⁹, R^{x}R^{y}N, R^{x}R^{y}NC(═O), R^{x}R^{y}NC₁₋₆alkyl, heteroaryl, aryl, C₁₋₆alkyl optionally substituted with up to 9 halo, C₁₋₆alkyl substituted with up to 5 hydroxy, C₁₋₆alkoxy optionally substituted with up to 9 halo, C₁₋₆haloalkyl, R^{a}R^{b}N—, (R^{a}R^{b}N)alkyl, (R^{a}R^{b}N)C(═O)—, C₁₋₆alkyl substituted with up to 5 hydroxy, said substituent aryl and heteroaryl are each optionally substituted with one or more R¹⁴, or optionally two vicinal R¹⁵ and the carbons to which they are attached are together a fused three- to six-membered carbocyclic ring optionally substituted with up to two C₁₋₆alkyl groups, or optionally two geminal R¹⁵ and the carbon to which they are attached are together a three- to six-membered carbocyclic ring optionally substituted with up to two C₁₋₆alkyl groups, or optionally two geminal R¹⁵ are together oxo;

each L⁵ is separately selected from the group consisting of

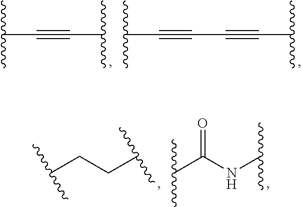

### —C(CF₃)₂NR^{2c}—,

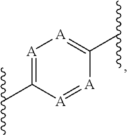

C(R²)₂, —C(R²)₂O—, —C(═O)—, O (oxygen), NH, and —(CH═CH)—;

R^{2c} is selected from the group consisting of hydrogen, C₁₋₆alkyl, C₂₋₆alkenyl, C₂₋₆alkynyl, C₃₋₇cycloalkyl, aryl, arylalkyl, heteroaryl, heteroarylalkyl, heterocyclyl, and heterocyclylalkyl, said alkyl optionally substituted with R^{e}R^{f}N—, alkoxy, or C₁₋₆alkylS—;

L⁶ is selected from the group consisting of

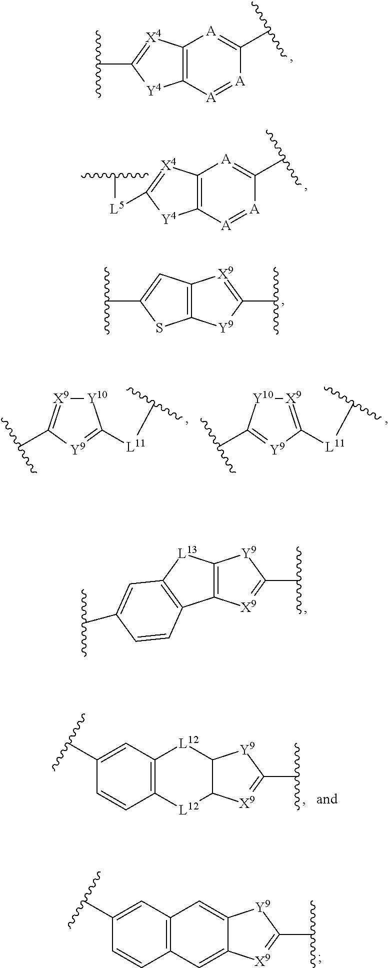

L⁷ is selected from the group consisting of

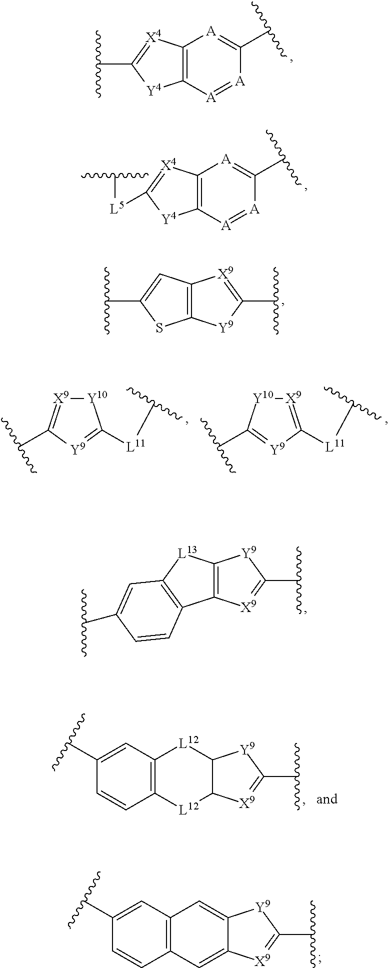

each X⁴ is separately selected from the group consisting of CR⁴ and N (nitrogen), wherein if X₄ is N (nitrogen) then Y₄ is not NH;

each X⁶ is separately selected from the group consisting of N (nitrogen), and CR⁸;

each Y⁴ is separately selected from the group consisting of C(R⁴)₂, NR⁴, O (oxygen), and S (sulfur);

each Y⁹ is separately selected from the group consisting of —NH—, O (oxygen), and S (sulfur);

each X⁹ is separately selected from the group consisting of CH and N (nitrogen), wherein if X⁹ is N (nitrogen) then Y⁹ is not NH;

each Y¹⁰ is separately selected from the group consisting of —CH₂— and —NH—;

each L¹¹ is separately selected from the group consisting of

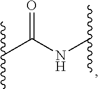

### —C(CF₃)₂NR^{2c}—, and NH;

each L¹² is separately selected from the group consisting of —CH₂— and —CH₂CH₂—;

each L¹³ is separately selected from the group consisting of —CH₂—, —N═CH—, —CH═CH—, —CH₂CH₂—, —(CH₂)ₘNR⁴(CH₂)ₙ— and —(CH₂)ₘO(CH₂)ₙ—;

each m separately is 1 or 2;

each n separately is 0, 1 or 2;

each p separately is 1, 2, 3 or 4;

each q separately is 1, 2, 3, 4 or 5;

each r separately is 0, 1, 2, 3, or 4;

each s separately is 0 or 1;

each R³ is separately selected from the group consisting of hydrogen, C₁₋₆alkoxy, C₁₋₆alkylOC₁₋₆alkyl, C₁₋₆alkylOC(═O)—, arylalkylOC(═O)—, —COOH, halo, hydroxy, R^{a}R^{b}N—, (R^{a}R^{b}N)alkyl, (R^{a}R^{b}N)C(═O)—, and C₁₋₆alkyl optionally substituted with up to 9 halo and up to 5 hydroxy;

each R⁴ is separately selected from the group consisting of H (hydrogen), C₁₋₆alkoxy, C₁₋₆alkylOC₁₋₆alkyl, C₁₋₆alkylOC(═O)—, arylalkylOC(═O)—, —COOH, halo, C₁₋₆haloalkyl, hydroxy, R^{a}R^{b}N—, (R^{a}R^{b}N)alkyl, (R^{a}R^{b}N)C(═O)—, and C₁₋₆alkyl optionally substituted with up to 9 halo and up to 5 hydroxy, or optionally two geminal R⁴ are together oxo;

R⁹ is selected from the group consisting of hydrogen and —C(═O)R^{9a};

R^{9a} is selected from the group consisting of —NR^{9b}R^{9c}, —OR^{9d}, C₁₋₆alkyl optionally substituted with up to 9 halo, and optionally substituted aryl;

R^{9b} is selected from the group consisting of hydrogen, C₁₋₆alkyl optionally substituted with up to 9 halo, and optionally substituted aryl;

R^{9c} is selected from the group consisting of C₁₋₆alkyl optionally substituted with up to 9 halo, and optionally substituted aryl; and

R^{9d} is selected from the group consisting of C₁₋₆alkyl optionally substituted with up to 9 halo, and optionally substituted aryl,

with the proviso that L⁶-L⁴-L⁷ is not

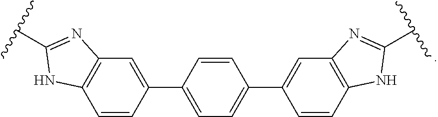

In some embodiments of Formula XII, L⁴ is selected from the group consisting of

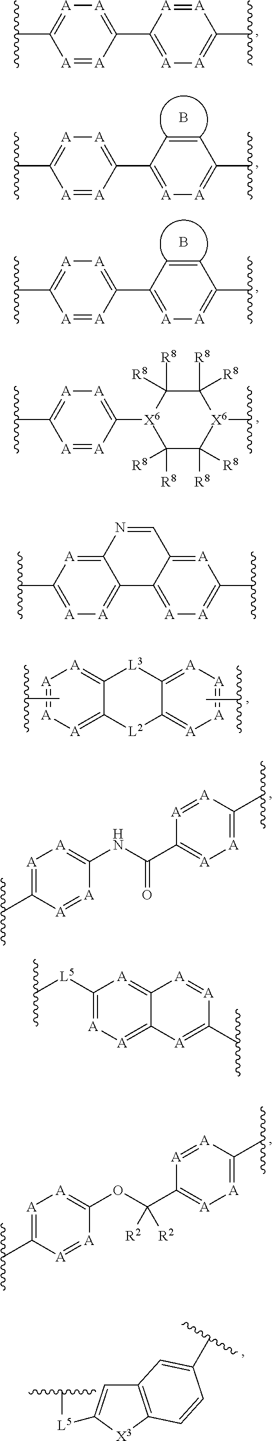

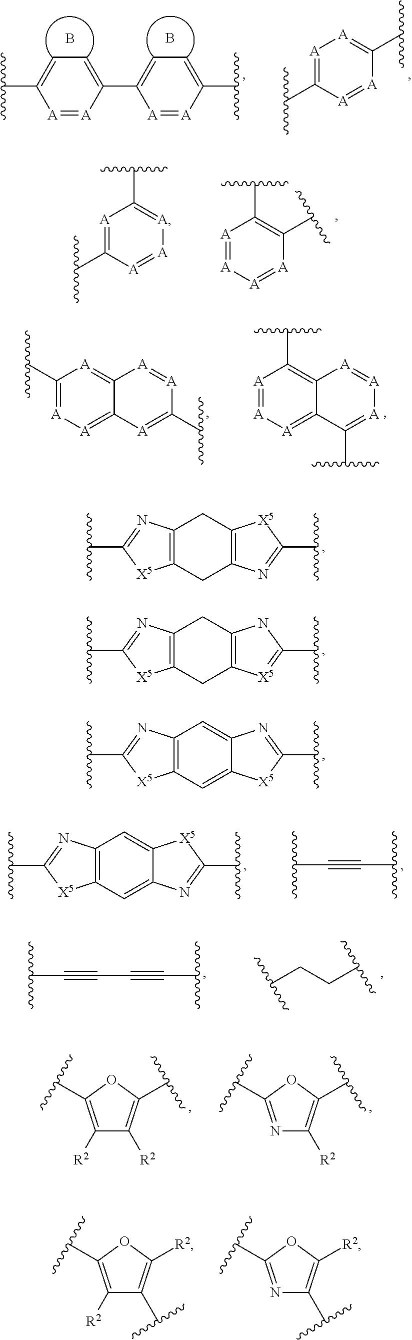

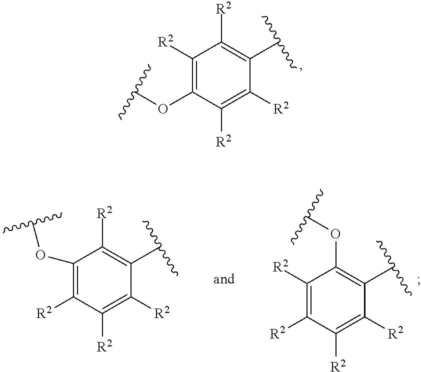

L² is selected from the group consisting of —C(═O)—, —(CH₂CH₂)—, —(CH₂O)—, —(CH₂S)—, —(CH═CH)—, —(CH═N)—, —NH—, O (oxygen), S (sulfur), and —CH₂—;

L³ is selected from the group consisting of

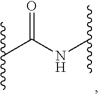

—(NR⁹)—, O (oxygen), S (sulfur), and —CH₂—;

L⁵ is selected from the group consisting of

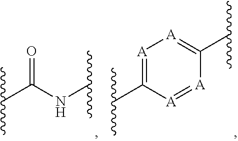

O (oxygen), NH, and —(CH═CH)—;

each X³ is separately selected from the group consisting of NH, O (oxygen), and S (sulfur);

each X⁵ is separately selected from the group consisting of —NH—, O (oxygen), S (sulfur), and —CH₂—;

each X⁶ is separately selected from the group consisting of N (nitrogen), and CR⁸;

each R⁸ is separately selected from the group consisting of hydrogen, C₁₋₆alkylOC₁₋₆alkyl, C₁₋₆alkylOC(═O)—, arylalkylOC(═O)—, halo, (R^{a}R^{b}N)alkyl, (R^{a}R^{b}N)C(═O)—, and C₁₋₆alkyl optionally substituted with up to 9 halo and up to 5 hydroxy;

each B is separately selected, wherein B is a fused optionally substituted saturated or unsaturated three- to seven-membered carbocyclic ring or a fused optionally substituted saturated or unsaturated three- to seven-membered heterocyclic ring, each optionally substituted with one or more R⁴; and

each R⁴ is separately selected from the group consisting of C₁₋₆alkoxy, C₁₋₆alkylOC₁₋₆alkyl, C₁₋₆alkylOC(═O)—, arylalkylOC(═O)—, —COOH, halo, C₁₋₆haloalkyl, hydroxy, R^{a}R^{b}N—, (R^{a}R^{b}N)alkyl, (R^{a}R^{b}N)C(═O)—, and C₁₋₆alkyl optionally substituted with up to 9 halo and up to 5 hydroxy, or optionally two geminal R⁴ are together oxo.

In some embodiments, the compound of Formula XII has the structure of Formula XIIa:

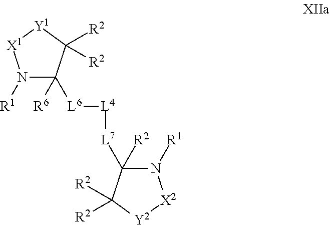

or a pharmaceutically acceptable salt thereof, wherein R⁶ is C₁₋₆alkyl optionally substituted with up to 9 halo. In some embodiments, R⁶ is methyl.

Some embodiments include a compound having the structure of Formula XIII:

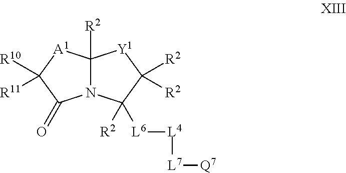

or a pharmaceutically acceptable salt thereof,

wherein:

each R¹ is separately selected from the group consisting of hydrogen, R^{1a}C(═O)— and R^{1a}C(═S)—;

each R^{1a} is separately selected from the group consisting of —C(R^{2a})₂NR^{3a}R^{3b}, alkoxyalkyl, C₁₋₆alkylOC(═O)—, C₁₋₆alkylOC(═O)C₁₋₆alkyl, C₁₋₆alkylC(═O)C₁₋₆alkyl, aryl, aryl(CH₂)ₙ—, aryl(CH₂)ₙO—, aryl(CH═CH)ₘ—, arylalkylO—, arylalkyl, arylOalkyl, cycloalkyl, (cycloalkyl)(CH═CH)ₘ—, (cycloalkyl)alkyl, cycloalkylOalkyl, heterocyclyl, heterocyclyl(CH═CH)ₘ—, heterocyclylalkoxy, heterocyclylalkyl, heterocyclylOalkyl, hydroxyalkyl, R^{c}R^{d}N—, R^{c}R^{d}N(CH₂)ₙ—, (R^{c}R^{d}N)(CH═CH)ₘ—, (R^{c}R^{d}N)alkyl, (R^{c}R^{d}N)C(═O)—, C₁₋₆alkoxy optionally substituted with up to 9 halo, and C₁₋₆alkyl optionally substituted with up to 9 halo, said aryl and heteroaryl each optionally substituted with cyano, halo, nitro, hydroxyl, C₁₋₆alkoxy optionally substituted with up to 9 halo, and C₁₋₆alkyl optionally substituted with up to 9 halo;

each C(R^{2a})₂ is separately selected, wherein each R^{2a} is separately selected from the group consisting of hydrogen, C₁₋₆alkyl optionally substituted with up to 9 halo, aryl(CH₂)ₙ—, and heteroaryl(CH₂)ₙ—, said aryl and heteroaryl each optionally substituted with cyano, halo, nitro, hydroxyl, C₁₋₆alkoxy optionally substituted with up to 9 halo, and C₁₋₆alkyl optionally substituted with up to 9 halo, or C(R^{2a})₂ is

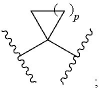

each R^{3a} is separately selected from the group consisting of hydrogen, and optionally substituted C₁₋₆alkyl;

each R^{3b} is separately selected from the group consisting of optionally substituted C₁₋₆alkyl, heteroaryl, —(CH₂)ₙC(═O)NR^{4a}R^{4b}, —(CH₂)ₙC(═O)OR^{5a}, and —(CH₂)ₙC(═O)R^{6a} said heteroaryl optionally substituted with cyano, halo, nitro, hydroxyl, C₁₋₆alkoxy optionally substituted with up to 9 halo, and C₁₋₆alkyl optionally substituted with up to 9 halo;

each R^{4a}R^{4b}N is separately selected, wherein R^{4a} and R^{4b} are each separately selected from the group consisting of hydrogen, optionally substituted C₁₋₆alkyl, and aryl(CH₂)ₙ—;

each R^{5a} is separately selected from the group consisting of optionally substituted C₁₋₆alkyl, and aryl(CH₂)ₙ—;

each R^{6a} is separately selected from the group consisting of optionally substituted C₁₋₆alkyl, and aryl(CH₂)ₙ—;

each R¹⁰ is R^{c}R^{d}N—;

each R¹¹ is separately selected from the group consisting of H (hydrogen), alkoxyalkyl, C₁₋₆alkylOC(═O)C₁₋₆alkyl, C₁₋₆alkylC(═O)C₁₋₆alkyl, aryl(CH₂)ₙ—, arylalkyl, arylOalkyl, cycloalkyl, (cycloalkyl)alkyl, cycloalkylOalkyl, heterocyclylalkyl, heterocyclylOalkyl, hydroxyalkyl, R^{c}R^{d}N(CH₂)ₙ—, (R^{c}R^{d}N)alkyl, and C₁₋₆alkyl optionally substituted with up to 9 halo;

each R^{c}R^{d}N is separately selected, wherein R^{c} and R^{d} are each separately selected from hydrogen, alkoxyC(═O)—, C₁₋₆alkyl, C₁₋₆alkylC(═O)—, C₁₋₆alkylsulfonyl, arylalkylOC(═O)—, arylalkyl, arylalkylC(═O)—, arylC(═O)—, arylsulfonyl, heterocyclylalkyl, heterocyclylalkylC(═O)—, heterocyclylC(═O)—, (R^{e}R^{f}N)alkyl, (R^{e}R^{f}N)alkylC(═O)—, and (R^{e}R^{f}N)C(═O)—, wherein the alkyl part of arylalkyl, arylalkylC(═O)—, heterocyclylalkyl, and heterocyclylalkylC(═O)— are each optionally substituted with one R^{e}R^{f}N— group; and wherein the aryl part of arylalkyl, arylalkylC(═O)—, arylC(═O)—, and arylsulfonyl, and the heterocyclyl part of heterocyclylalkyl, heterocyclylalkylC(═O)—, and heterocyclylC(═O)— are each optionally substituted with up to three substituents each independently selected from the group consisting of cyano, halo, nitro, C₁₋₆alkoxy optionally substituted with up to 9 halo, and C₁₋₆alkyl optionally substituted with up to 9 halo;

each R^{e}R^{f}N is separately selected, wherein R^{e} and R^{f} are each separately selected from hydrogen, C₁₋₆alkyl, aryl, arylalkyl, cycloalkyl, (cyclolalkyl)alkyl, heterocyclyl, heterocyclylalkyl, (R^{x}R^{y}N)alkyl, and (R^{x}R^{y}N)C(═O)—;

each R^{x}R^{y}N is separately selected, wherein R^{x} and R^{y} are each separately selected from hydrogen, C₁₋₆alkylOC(═O)—, C₁₋₆alkyl, C₁₋₆alkylC(═O)—, aryl, arylalkyl, cycloalkyl, and heterocyclyl;

Y¹ is selected from O (oxygen), S (sulfur), S(O), SO₂, NR², and C(R²)₂;

each A¹ is separately selected from the group consisting of C₂₋₆alkenyl, C₁₋₆alkyl, and —(CH₂)ₙ—O—(CH₂)ₘ—, each optionally substituted with one or more R²;

each R² is separately selected, wherein R² is selected from the group consisting of hydrogen, C₁₋₆alkoxy, C₁₋₆alkyl, aryl, halo, hydroxy, R^{a}R^{b}N—, and C₁₋₆alkyl optionally substituted with up to 9 halo, or optionally two vicinal R² and the carbons to which they are attached are together a fused three- to six-membered carbocyclic ring optionally substituted with up to two C₁₋₆alkyl groups, or optionally two geminal R² and the carbon to which they are attached are together a three- to six-membered carbocyclic ring optionally substituted with up to two C₁₋₆alkyl groups;

L⁴ is selected from the group consisting of -(J²)ₛ-(L⁵)ₛ-(J²)ₛ-(L⁵)ₛ-J²-,

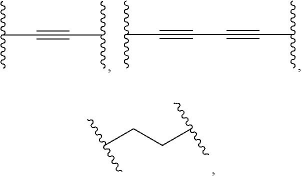

, —C(═O)—, O (oxygen), —OC(R²)₂—,

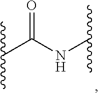

### —C(CF₃)₂NR^{2c}—, NH, and —(CH═CH)—;

J² is aryl, heteroaryl, heterocyclyl, cycloalkyl, cycloalkenyl, or polycyclic hydrocarbon, each optionally substituted with one or more R¹⁵;

each R¹⁴ is separately selected from the group consisting of hydroxy, C₁₋₆alkoxy optionally substituted with up to 9 fluoro, C₁₋₆alkylOC₁₋₆alkyl, C₁₋₆alkylOC(═O)—, arylalkylOC(═O)—, —COOH, halo, and C₁₋₆alkyl optionally substituted with up to 9 halo;

each R¹⁵ is separately selected from the group consisting of halo, hydroxy, C₁₋₆alkylOC₁₋₆alkyl, C₁₋₆alkylOC(═O)—, arylalkylOC(═O)—, —COOH, R⁹, R^{x}R^{y}N, R^{x}R^{y}NC(═O), R^{x}R^{y}NC₁₋₆alkyl, heteroaryl, aryl, C₁₋₆alkyl optionally substituted with up to 9 halo, C₁₋₆alkoxy optionally substituted with up to 9 halo, C₁₋₆haloalkyl, R^{a}R^{b}N—, (R^{a}R^{b}N)alkyl, (R^{a}R^{b}N)C(═O)—, and C₁₋₆alkyl substituted with up to 5 hydroxy, said substituent aryl and heteroaryl are each optionally substituted with one or more R¹⁴, or optionally two vicinal R¹⁵ and the carbons to which they are attached are together a fused three- to six-membered carbocyclic ring optionally substituted with up to two C₁₋₆alkyl groups, or optionally two geminal R¹⁵ and the carbon to which they are attached are together a three- to six-membered carbocyclic ring optionally substituted with up to two C₁₋₆alkyl groups, or optionally two geminal R¹⁵ are together oxo;

each R^{a}R^{b}N is separately selected, wherein R^{a} and R^{b} are each separately selected from the group consisting of hydrogen, C₂₋₆alkenyl, and C₁₋₆alkyl;

each L⁵ is separately selected from the group consisting of

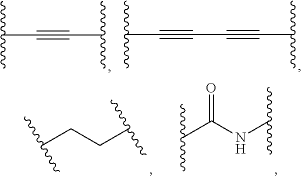

### —C(CF₃)₂NR^{2c}—,

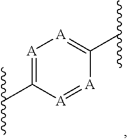

—C(R²)₂, —C(R²)₂O—, —C(═O)—, O (oxygen), NH, and —(CH═CH)—;

L⁶ is selected from the group consisting of

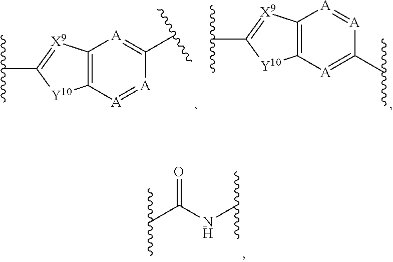

### —C(CF₃)₂NR^{2c}—,

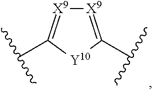

and

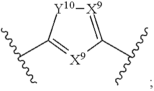

L⁷ is selected from the group consisting of

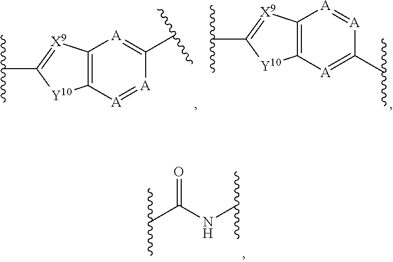

### —C(CF₃)₂NR^{2c}—,

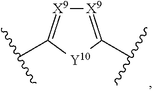

and

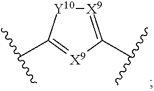

each A is separately selected from the group consisting of CR³ and N (nitrogen);

each R³ is separately selected from the group consisting of hydrogen, C₁₋₆alkoxy, C₁₋₆alkylOC₁₋₆alkyl, C₁₋₆alkylOC(═O)—, arylalkylOC(═O)—, —COOH, halo, hydroxy, R^{a}R^{b}N—, (R^{a}R^{b}N)alkyl, (R^{a}R^{b}N)C(═O)—, and C₁₋₆alkyl optionally substituted with up to 9 halo and up to 5 hydroxy;

R^{2c} is selected from the group consisting of hydrogen, C₁₋₆alkyl, C₂₋₆alkenyl, C₂₋₆alkynyl, C₃₋₇cycloalkyl, aryl, arylalkyl, heteroaryl, heteroarylalkyl, heterocyclyl, and heterocyclylalkyl, said alkyl optionally substituted with R^{e}R^{f}N—, alkoxy, or C₁₋₆alkylS—;

each X⁹ is separately selected from the group consisting of CH and N (nitrogen);

each X¹⁰ is (C(R²)₂)_{q};

each Y¹⁰ is separately selected from the group consisting of —CH₂— and —NH—;

each Y¹¹ is separately selected from the group consisting of —O(C(R²)₂)ₙ—, —S(C(R²)₂)ₙ—, —S(O)(C(R²)₂)ₙ—, —SO₂(C(R²)₂)ₙ—, —NR²(C(R²)₂)ₙ—, and (C(R²)₂)_{q};

each m separately is 1 or 2;

each n separately is 0, 1 or 2;

each p separately is 1, 2, 3 or 4;

each q separately is 1, 2, 3, 4 or 5;

each r separately is 0, 1, 2, 3, or 4;

each s separately is 0 or 1;

Q⁷ is selected from the group consisting of J²,

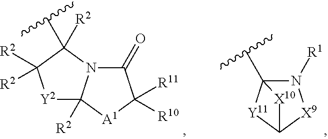

and

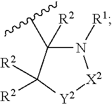

X² is (C(R²)₂)_{q},

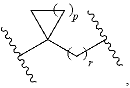

or X₂ is null;

Y² is selected from O (oxygen), S (sulfur), S(O), SO₂, NR², and C(R²)₂;

R⁹ is selected from the group consisting of hydrogen and —C(═O)R^{9a};

R^{9a} is selected from the group consisting of —NR^{9b}R^{9c}, —OR^{9d}, C₁₋₆alkyl optionally substituted with up to 9 halo, and optionally substituted aryl;

R^{9b} is selected from the group consisting of hydrogen, C₁₋₆alkyl optionally substituted with up to 9 halo, and optionally substituted aryl;

R^{9c} is selected from the group consisting of C₁₋₆alkyl optionally substituted with up to 9 halo, and optionally substituted aryl; and

R^{9d} is selected from the group consisting of C₁₋₆alkyl optionally substituted with up to 9 halo, and optionally substituted aryl.

In some embodiments of Formula XIII the compound is not selected from the group consisting of:

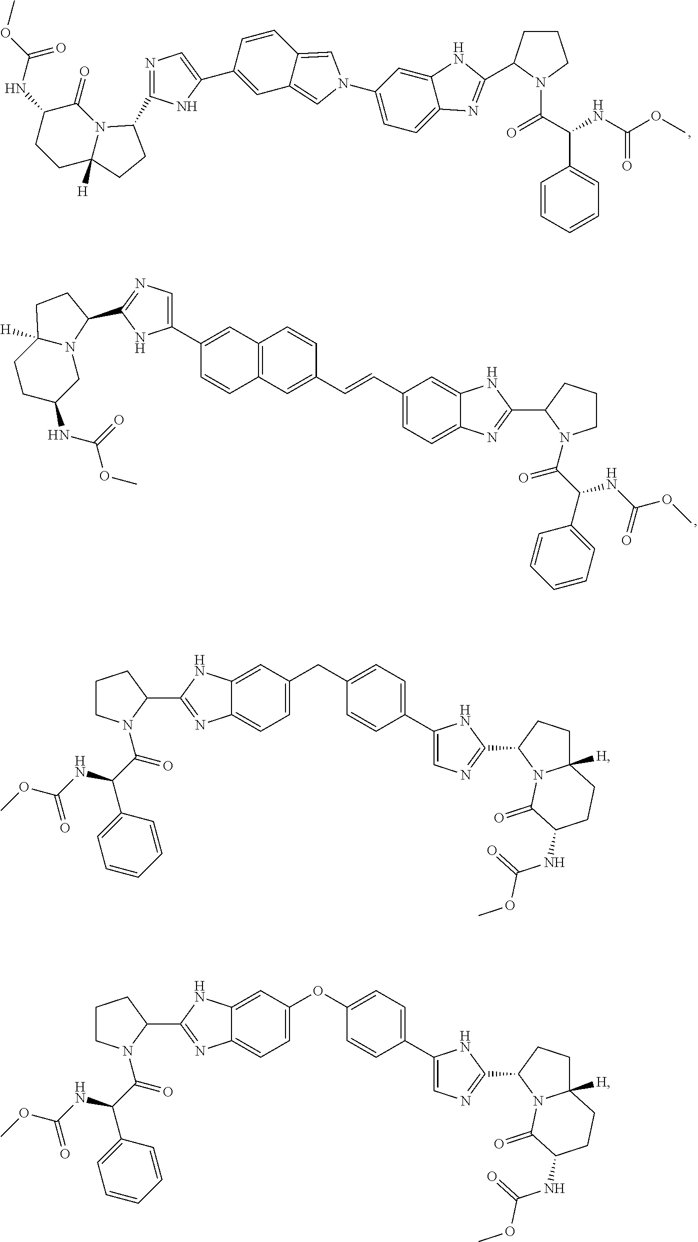

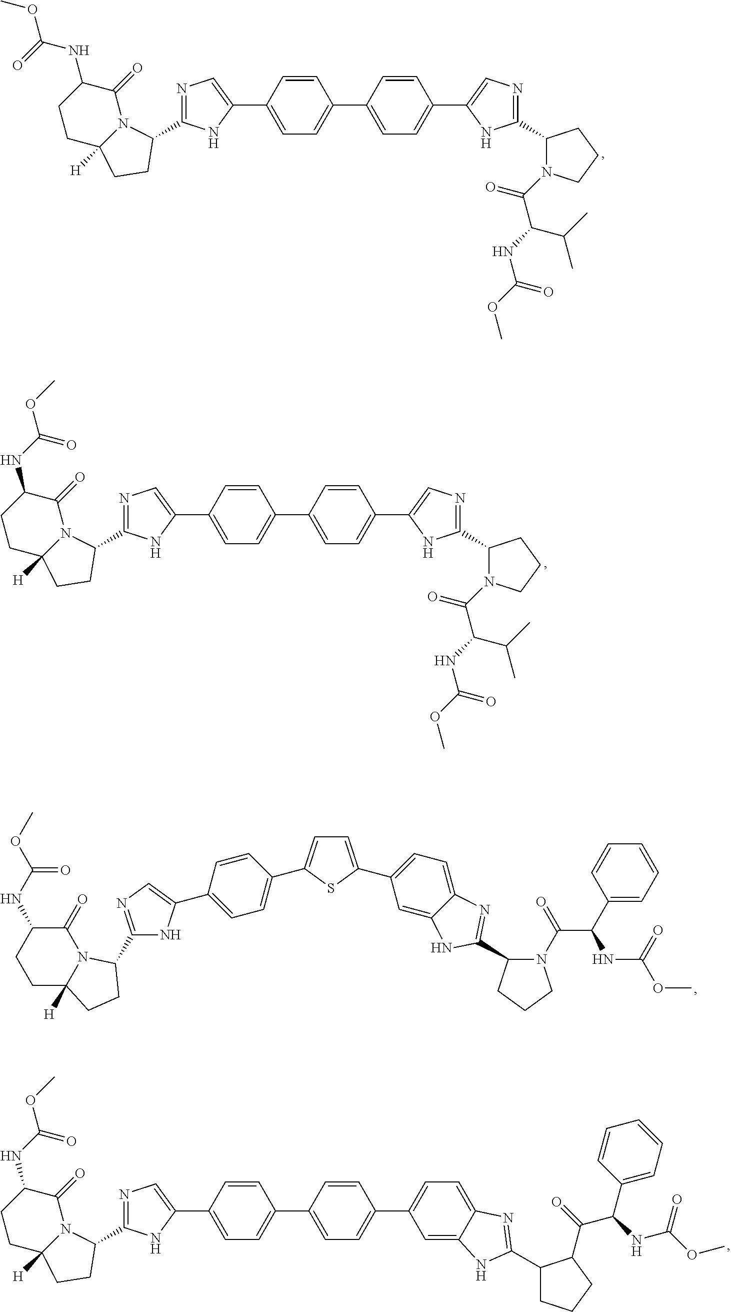

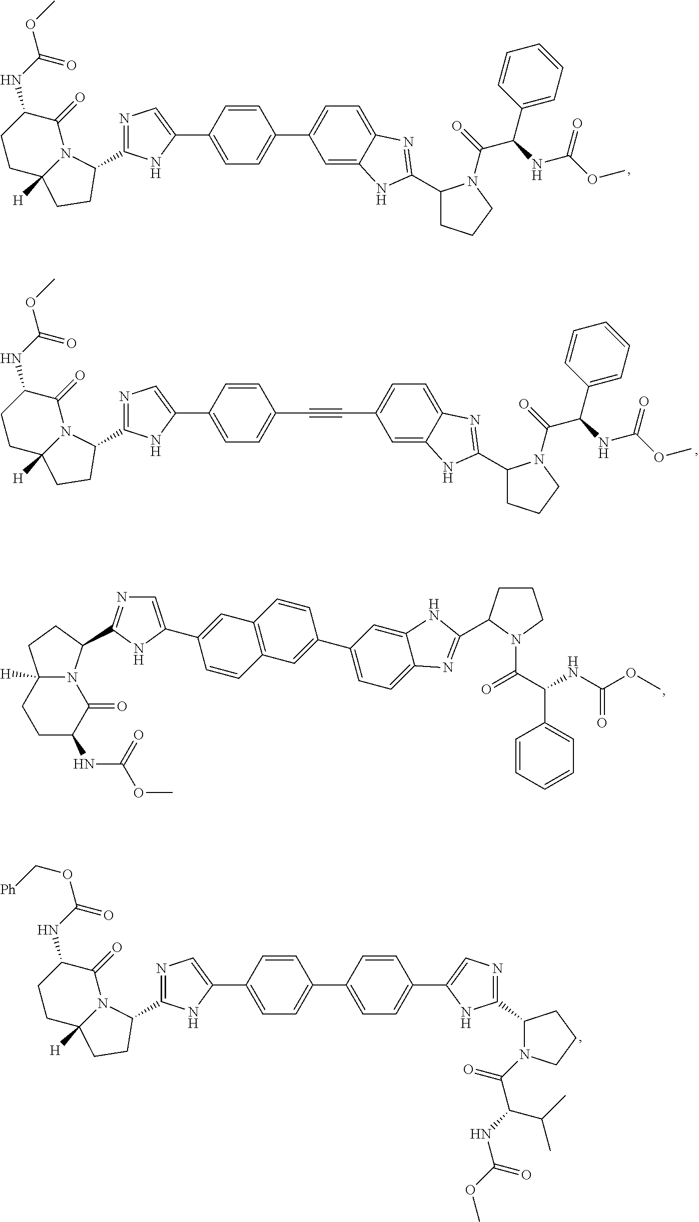

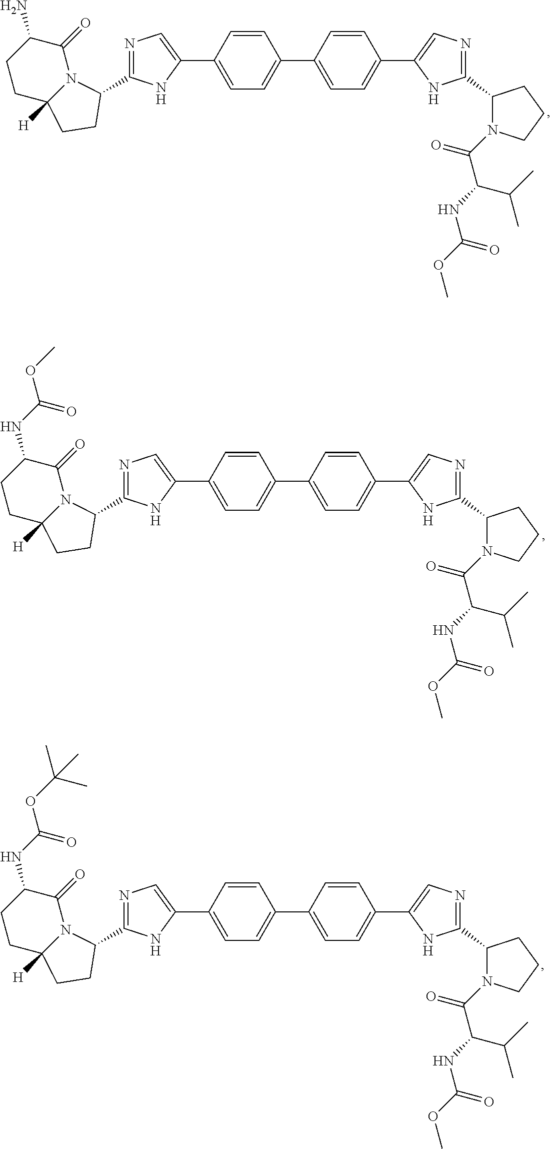

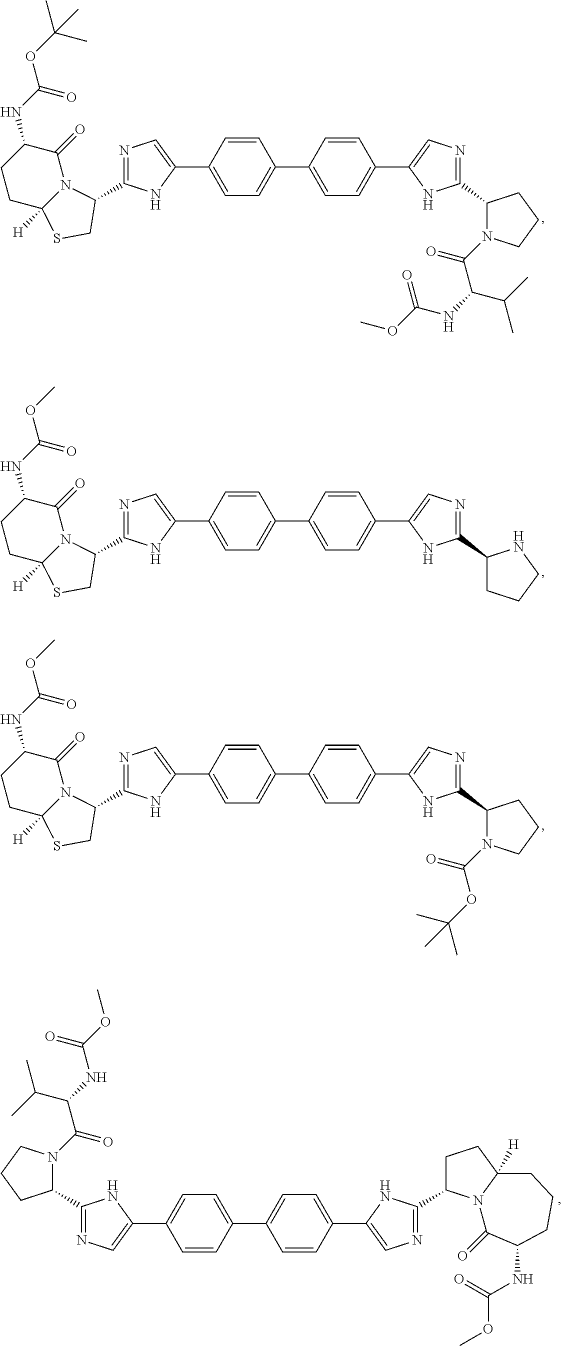

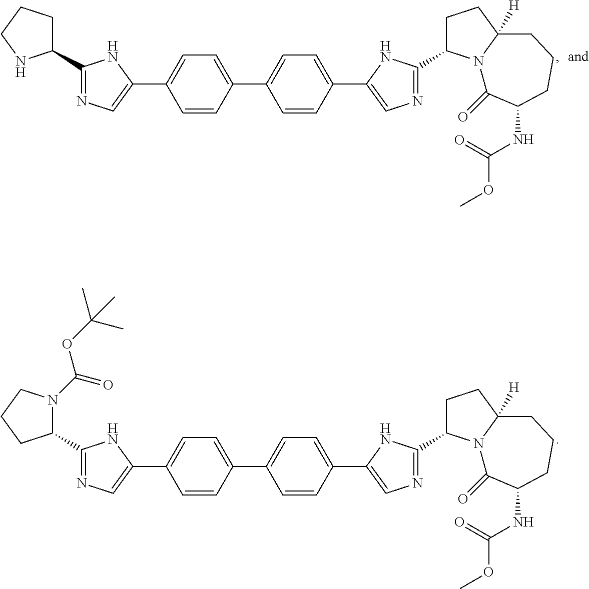

In some embodiments of Formula XIII, L⁴ is selected from the group consisting of

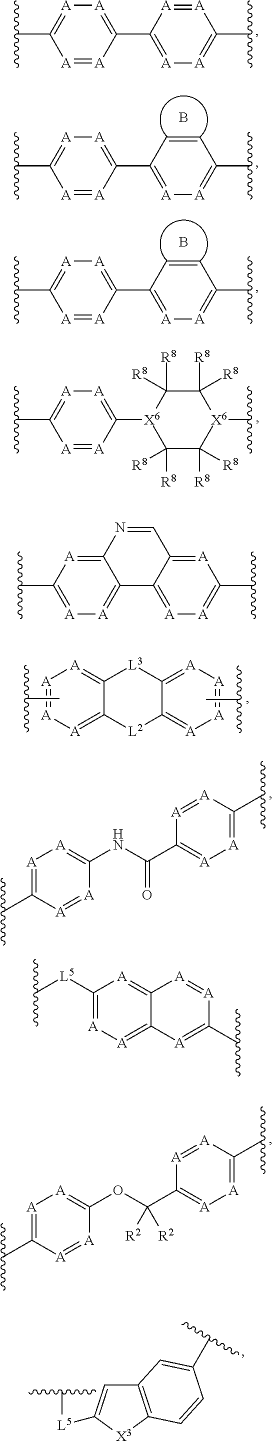

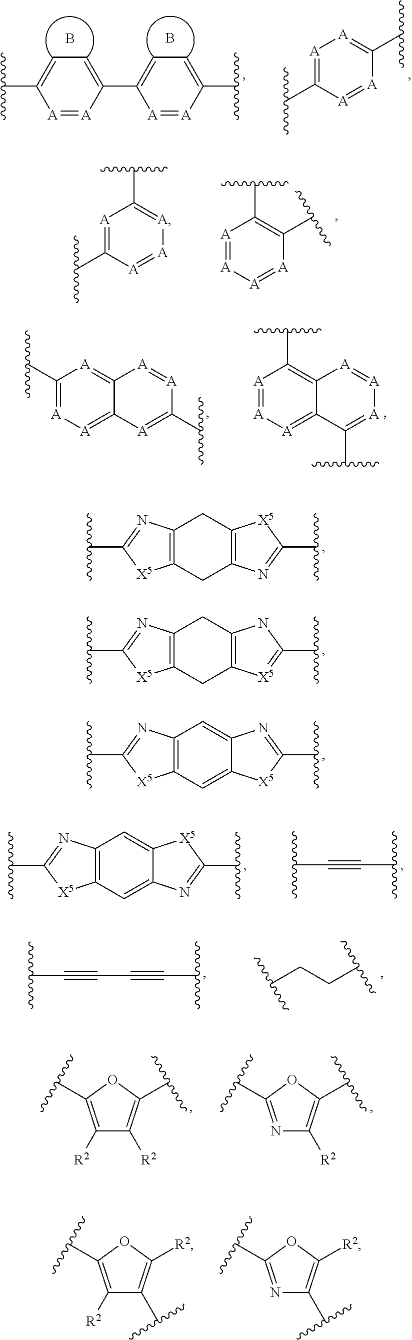

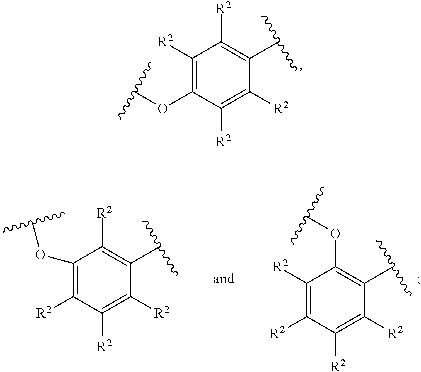

L² is selected from the group consisting of —C(═O)—, —(CH₂CH₂)—, —(CH₂O)—, —(CH₂S)—, —(CH═CH)—, —(CH═N)—, —NH—, O (oxygen), S (sulfur), and —CH₂—;

L³ is selected from the group consisting of

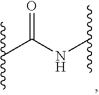

—(NR⁹)—, O (oxygen), S (sulfur), and —CH₂—;

L⁵ is selected from the group consisting of

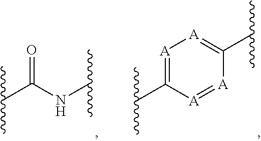

O (oxygen), NH, and —(CH═CH)—;

each X³ is separately selected from the group consisting of NH, O (oxygen), and S (sulfur);

each X⁵ is separately selected from the group consisting of —NH—, O (oxygen), S (sulfur), and —CH₂—;

each X⁶ is separately selected from the group consisting of N (nitrogen), and CR⁸;

each R⁸ is separately selected from the group consisting of hydrogen, C₁₋₆alkylOC₁₋₆alkyl, C₁₋₆alkylOC(═O)—, arylalkylOC(═O)—, halo, (R^{a}R^{b}N)alkyl, (R^{a}R^{b}N)C(═O)—, and C₁₋₆alkyl optionally substituted with up to 9 halo and up to 5 hydroxy;

each B is separately selected, wherein B is a fused optionally substituted saturated or unsaturated three- to seven-membered carbocyclic ring or a fused optionally substituted saturated or unsaturated three- to seven-membered heterocyclic ring, each optionally substituted with one or more R⁴; and

each R⁴ is separately selected from the group consisting of C₁₋₆alkoxy, C₁₋₆alkylOC₁₋₆alkyl, C₁₋₆alkylOC(═O)—, arylalkylOC(═O)—, —COOH, halo, C₁₋₆haloalkyl, hydroxy, R^{a}R^{b}N—, (R^{a}R^{b}N)alkyl, (R^{a}R^{b}N)C(═O)—, and C₁₋₆alkyl optionally substituted with up to 9 halo and up to 5 hydroxy, or optionally two geminal R⁴ are together oxo.

In some embodiments of Formula XIII, Q⁷ is

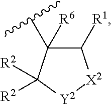

and R⁶ is C₁₋₆alkyl optionally substituted with up to 9 halo. In some embodiments, R⁶ is methyl.

In some embodiments of Formula XIII, L⁴ is selected from the group consisting of O (oxygen), —OC(R²)₂—,

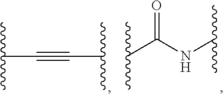

### —C(CF₃)₂NR^{2c}—,

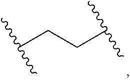

### NH, —(CH═CH)—, Y⁶, Y⁶—Y⁶, and Y⁶—Y⁶—Y⁶;

each Y⁶ is separately selected from the group consisting of aryl, heteroaryl, heterocyclyl, and polycyclic hydrocarbon, each optionally substituted with one or more substituents selected from the groups consisting of R², R³, R⁴, and R⁸;

each R² is separately selected, wherein R² is selected from the group consisting of C₁₋₆alkoxy, C₁₋₆alkyl, aryl, halo, hydroxy, R^{a}R^{b}N—, and C₁₋₆alkyl optionally substituted with up to 9 halo, or optionally two vicinal R² and the carbons to which they are attached are together a fused three- to six-membered carbocyclic ring optionally substituted with up to two C₁₋₆alkyl groups, or optionally two geminal R² and the carbon to which they are attached are together a three- to six-membered carbocyclic ring optionally substituted with up to two C₁₋₆alkyl groups;

each R³ is separately selected from the group consisting of C₁₋₆alkoxy, C₁₋₆alkylOC₁₋₆alkyl, C₁₋₆alkylOC(═O)—, arylalkylOC(═O)—, —COOH, halo, hydroxy, R^{a}R^{b}N—, (R^{a}R^{b}N)alkyl, (R^{a}R^{b}N)C(═O)—, and C₁₋₆alkyl optionally substituted with up to 9 halo and up to 5 hydroxy;

each R⁴ is separately selected from the group consisting of C₁₋₆alkoxy, C₁₋₆alkylOC₁₋₆alkyl, C₁₋₆alkylOC(═O)—, arylalkylOC(═O)—, —COOH, halo, C₁₋₆haloalkyl, hydroxy, R^{a}R^{b}N—, (R^{a}R^{b}N)alkyl, (R^{a}R^{b}N)C(═O)—, and C₁₋₆alkyl optionally substituted with up to 9 halo and up to 5 hydroxy, or optionally two geminal R⁴ are together oxo; and

each R⁸ is separately selected from the group consisting of C₁₋₆alkylOC₁₋₆alkyl, C₁₋₆alkylOC(═O)—, arylalkylOC(═O)—, halo, (R^{a}R^{b}N)alkyl, (R^{a}R^{b}N)C(═O)—, and C₁₋₆alkyl optionally substituted with up to 9 halo and up to 5 hydroxy.

In some embodiments, the compound Formula XIII has the structure

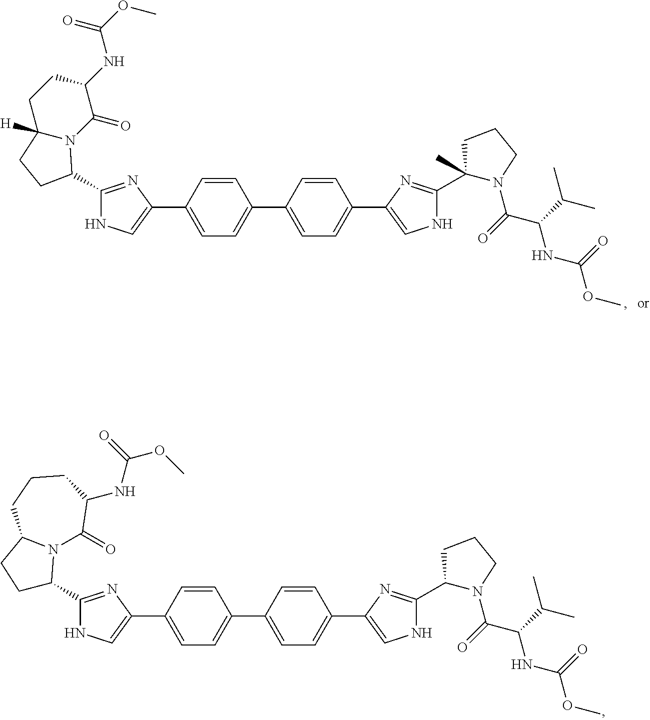

or a pharmaceutically acceptable salt thereof.

Some embodiments include a compound having the structure of Formula XIV:

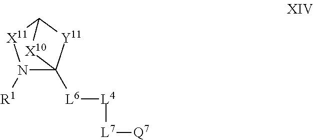

or a pharmaceutically acceptable salt thereof,

wherein:

Q⁷ is selected from the group consisting of J²,

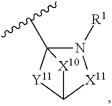

and

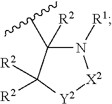

each X¹⁰ is (C(R²)₂)_{q};

each X¹¹ is separately selected from the group consisting of (C(R²)₂)_{q}, and

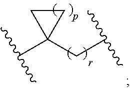

each Y¹¹ is separately selected from the group consisting of —O(C(R²)₂)ₙ—, —S(C(R²)₂)ₙ—, —S(O)(C(R²)₂)ₙ—, —SO₂(C(R²)₂)ₙ—, —NR²(C(R²)₂)ₙ—, and (C(R²)₂)_{q};

each R¹ is separately selected from the group consisting of hydrogen, R^{1a}C(═O)— and R^{1a}C(═S)—;

each R^{1a} is separately selected from the group consisting of —C(R^{2a})₂NR^{3a}R^{3b}, alkoxyalkyl, C₁₋₆alkylOC(═O)—, C₁₋₆alkylOC(═O)C₁₋₆alkyl, C₁₋₆alkylC(═O)C₁₋₆alkyl, aryl, aryl(CH₂)ₙ—, aryl(CH₂)ₙO—, aryl(CH═CH)ₘ—, arylalkylO—, arylalkyl, arylOalkyl, cycloalkyl, (cycloalkyl)(CH═CH)ₘ—, (cycloalkyl)alkyl, cycloalkylOalkyl, heterocyclyl, heterocyclyl(CH═CH)ₘ—, heterocyclylalkoxy, heterocyclylalkyl, heterocyclylOalkyl, hydroxyalkyl, R^{c}R^{d}N—, R^{c}R^{d}N(CH₂)ₙ—, (R^{c}R^{d}N)(CH═CH)ₘ—, (R^{c}R^{d}N)alkyl, (R^{c}R^{d}N)C(═O)—, C₁₋₆alkoxy optionally substituted with up to 9 halo, and C₁₋₆alkyl optionally substituted with up to 9 halo, said aryl and heteroaryl each optionally substituted with cyano, halo, nitro, hydroxyl, C₁₋₆alkoxy optionally substituted with up to 9 halo, and C₁₋₆alkyl optionally substituted with up to 9 halo;

each R^{c}R^{d}N is separately selected, wherein R^{c} and R^{d} are each separately selected from hydrogen, alkoxyC(═O)—, C₁₋₆alkyl, C₁₋₆alkylC(═O)—, C₁₋₆alkylsulfonyl, arylalkylOC(═O)—, arylalkyl, arylalkylC(═O)—, arylC(═O)—, arylsulfonyl, heterocyclylalkyl, heterocyclylalkylC(═O)—, heterocyclylC(═O)—, (R^{e}R^{f}N)alkyl, (R^{e}R^{f}N)alkylC(═O)—, and (R^{e}R^{f}N)C(═O)—, wherein the alkyl part of arylalkyl, arylalkylC(═O)—, heterocyclylalkyl, and heterocyclylalkylC(═O)— are each optionally substituted with one R^{e}R^{f}N— group; and wherein the aryl part of arylalkyl, arylalkylC(═O)—, arylC(═O)—, and arylsulfonyl, and the heterocyclyl part of heterocyclylalkyl, heterocyclylalkylC(═O)—, and heterocyclylC(═O)— are each optionally substituted with up to three substituents each independently selected from the group consisting of cyano, halo, nitro, C₁₋₆alkoxy optionally substituted with up to 9 halo, and C₁₋₆alkyl optionally substituted with up to 9 halo;

each R^{e}R^{f}N is separately selected, wherein R^{e} and R^{f} are each separately selected from hydrogen, C₁₋₆alkyl, aryl, arylalkyl, cycloalkyl, (cyclolalkyl)alkyl, heterocyclyl, heterocyclylalkyl, (R^{x}R^{y}N)alkyl, and (R^{x}R^{y}N)C(═O)—;

each R^{x}R^{y}N is separately selected, wherein R^{x} and R^{y} are each separately selected from hydrogen, C₁₋₆alkylOC(═O)—, C₁₋₆alkyl, C₁₋₆alkylC(═O)—, aryl, arylalkyl, cycloalkyl, and heterocyclyl;

each C(R^{2a})₂ is separately selected, wherein each R^{2a} is separately selected from the group consisting of hydrogen, C₁₋₆alkyl optionally substituted with up to 9 halo, aryl(CH₂)ₙ—, and heteroaryl(CH₂)ₙ—, said aryl and heteroaryl each optionally substituted with cyano, halo, nitro, hydroxyl, C₁₋₆alkoxy optionally substituted with up to 9 halo, and C₁₋₆alkyl optionally substituted with up to 9 halo, or C(R^{2a})₂ is

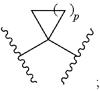

each R^{3a} is separately selected from the group consisting of hydrogen, and optionally substituted C₁₋₆alkyl;

each R^{3b} is separately selected from the group consisting of optionally substituted C₁₋₆alkyl, heteroaryl, —(CH₂)ₙC(═O)NR^{4a}R^{4b}, —(CH₂)ₙC(═O)OR^{5a}, and —(CH₂)ₙC(═O)R^{6a} said heteroaryl optionally substituted with cyano, halo, nitro, hydroxyl, C₁₋₆alkoxy optionally substituted with up to 9 halo, and C₁₋₆alkyl optionally substituted with up to 9 halo;

each R^{4a}R^{4b}N is separately selected, wherein R^{4a} and R^{4b} are each separately selected from the group consisting of hydrogen, optionally substituted C₁₋₆alkyl, and aryl(CH₂)ₙ—;

each R^{5a} is separately selected from the group consisting of optionally substituted C₁₋₆alkyl, and aryl(CH₂)ₙ—;

each R^{6a} is separately selected from the group consisting of optionally substituted C₁₋₆alkyl, and aryl(CH₂)ₙ—;

X¹ is (C(R²)₂)_{q},

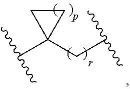

or X¹ is null;

Y¹ is selected from O (oxygen), S (sulfur), S(O), SO₂, NR², and C(R²)₂ with the proviso that when X¹ is null Y¹ is C(R²)₂;

X² is (C(R²)₂)_{q},

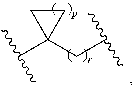

or X² is null;

Y² is selected from O (oxygen), S (sulfur), S(O), SO₂, NR², and C(R²)₂ with the proviso that when X² is null Y² is C(R²)₂;

each R² is separately selected, wherein R² is selected from the group consisting of hydrogen, C₁₋₆alkoxy, C₁₋₆alkyl, aryl, halo, hydroxy, R^{a}R^{b}N—, and C₁₋₆alkyl optionally substituted with up to 9 halo, or optionally two vicinal R² and the carbons to which they are attached are together a fused three- to six-membered carbocyclic ring optionally substituted with up to two C₁₋₆alkyl groups;

each R^{a}R^{b}N is separately selected, wherein R^{a} and R^{b} are each separately selected from the group consisting of hydrogen, C₂₋₆alkenyl, and C₁₋₆alkyl;

L⁴ is selected from the group consisting of -(J²)ₛ-(L⁵)ₛ-(J²)ₛ-(L⁵)ₛ-J²-,

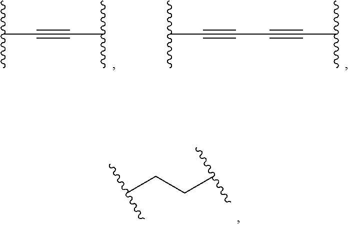

—C(═O)—, O (oxygen), —OC(R²)₂—,

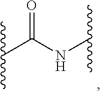

### —C(CF₃)₂NR^{2c}—, NH, and —(CH═CH)—;

J² is aryl, heteroaryl, heterocyclyl, cycloalkyl, cycloalkenyl, or polycyclic hydrocarbon, each optionally substituted with one or more R¹⁵;

each R¹⁴ is separately selected from the group consisting of hydroxy, C₁₋₆alkoxy optionally substituted with up to 9 fluoro, C₁₋₆alkylOC₁₋₆alkyl, C₁₋₆alkylOC(═O)—, arylalkylOC(═O)—, —COOH, halo, and C₁₋₆alkyl optionally substituted with up to 9 halo;

each R¹⁵ is separately selected from the group consisting of halo, hydroxy, C₁₋₆alkylOC₁₋₆alkyl, C₁₋₆alkylOC(═O)—, arylalkylOC(═O)—, —COOH, R⁹, R^{x}R^{y}N, R^{x}R^{y}NC(═O), R^{x}R^{y}NC₁₋₆alkyl, heteroaryl, aryl, C₁₋₆alkyl optionally substituted with up to 9 halo, C₁₋₆alkyl substituted with up to 5 hydroxy, C₁₋₆alkoxy optionally substituted with up to 9 halo, C₁₋₆haloalkyl, R^{a}R^{b}N—, (R^{a}R^{b}N)alkyl, (R^{a}R^{b}N)C(═O)—, and C₁₋₆alkyl substituted with up to 5 hydroxy, said substituent aryl and heteroaryl are each optionally substituted with one or more R¹⁴, or optionally two vicinal R¹⁵ and the carbons to which they are attached are together a fused three- to six-membered carbocyclic ring optionally substituted with up to two C₁₋₆alkyl groups, or optionally two geminal R¹⁵ and the carbon to which they are attached are together a three- to six-membered carbocyclic ring optionally substituted with up to two C₁₋₆alkyl groups, or optionally two geminal R¹⁵ are together oxo;

each L⁵ is separately selected from the group consisting of

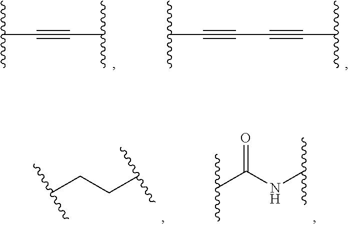

### —C(CF₃)₂NR^{2c}—,

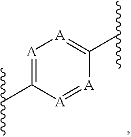

—C(R²)₂, —C(R²)₂O—, —C(═O)—, O (oxygen), NH, and —(CH═CH)—;

each A is separately selected from the group consisting of CR³ and N (nitrogen);

R^{2c} is selected from the group consisting of hydrogen, C₁₋₆alkyl, C₂₋₆alkenyl, C₂₋₆alkynyl, C₃₋₇cycloalkyl, aryl, arylalkyl, heteroaryl, heteroarylalkyl, heterocyclyl, and heterocyclylalkyl, said alkyl optionally substituted with R^{e}R^{f}N—, alkoxy, or C₁₋₆alkylS—;

L⁶ is selected from the group consisting of

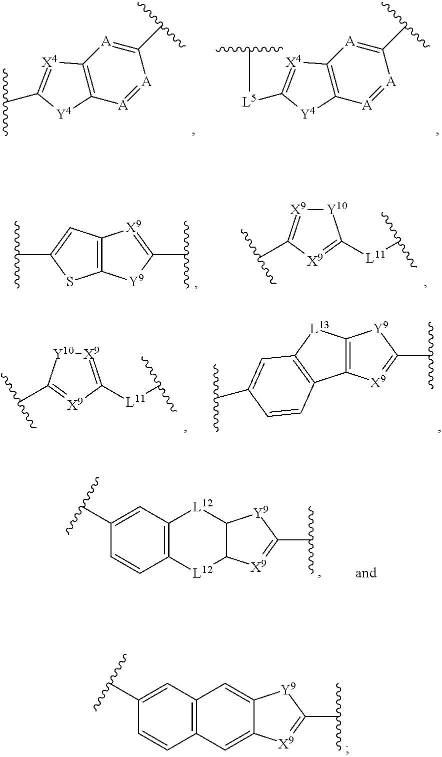

L⁷ is selected from the group consisting of

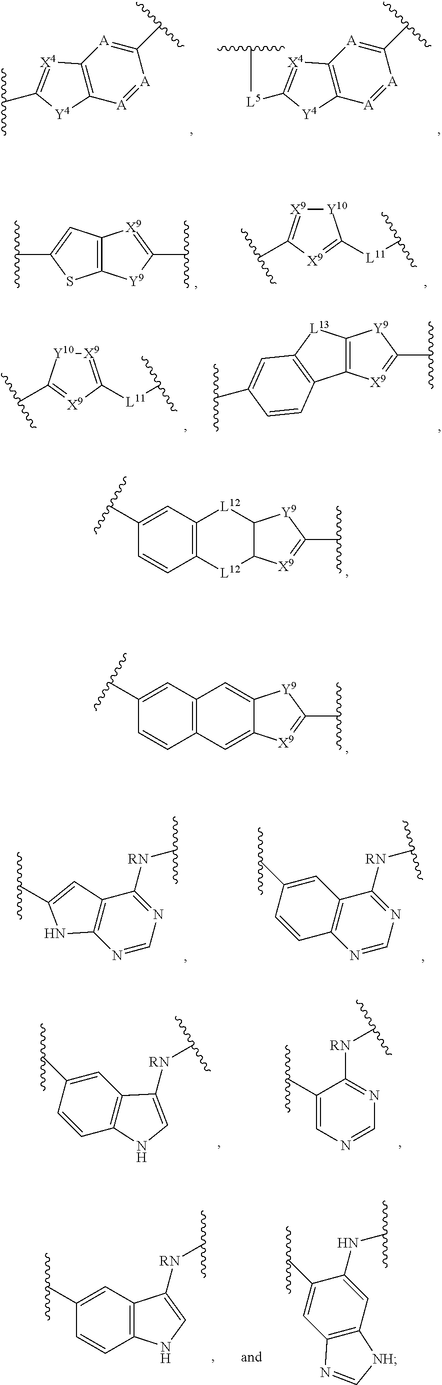

each R is separately selected from the group consisting of hydrogen, C₁₋₆alkylOC(═O)—, arylalkylOC(═O)—, (R^{a}R^{b}N)C(═O)—, and C₁₋₆alkyl optionally substituted with up to 9 halo;

each X⁴ is separately selected from the group consisting of CR⁴ and N (nitrogen), wherein if X⁴ is N (nitrogen) then Y⁴ is not NH;

each Y⁴ is separately selected from the group consisting of C(R⁴)₂, NR⁴, O (oxygen), and S (sulfur);

each Y⁹ is separately selected from the group consisting of —NH—, O (oxygen), and S (sulfur);

each X⁹ is separately selected from the group consisting of CH and N (nitrogen), wherein if X⁹ is N (nitrogen) then Y⁹ is not NH;

each Y¹⁰ is separately selected from the group consisting of —CH₂— and —NH—;

each L¹¹ is separately selected from the group consisting of

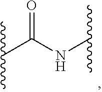

### and NH;

each L¹² is separately selected from the group consisting of —CH₂— and —CH₂CH₂—;

each L¹³ is separately selected from the group consisting of —CH₂—, —N═CH—, —CH═CH—, —CH₂CH₂—, —(CH₂)ₘNR⁴(CH₂)ₙ— and —(CH₂)ₘO(CH₂)ₙ—;

each m separately is 1 or 2;

each n separately is 0, 1 or 2;

each p separately is 1, 2, 3 or 4;

each q separately is 1, 2, 3, 4 or 5;

each r separately is 0, 1, 2, 3, or 4;

each s separately is 0 or 1;

each R³ is separately selected from the group consisting of hydrogen, C₁₋₆alkoxy, C₁₋₆alkylOC₁₋₆alkyl, C₁₋₆alkylOC(═O)—, arylalkylOC(═O)—, —COOH, halo, hydroxy, R^{a}R^{b}N—, (R^{a}R^{b}N)alkyl, (R^{a}R^{b}N)C(═O)—, and C₁₋₆alkyl optionally substituted with up to 9 halo and up to 5 hydroxy; and

each R⁴ is separately selected from the group consisting of H (hydrogen), C₁₋₆alkoxy, C₁₋₆alkylOC₁₋₆alkyl, C₁₋₆alkylOC(═O)—, arylalkylOC(═O)—, —COOH, halo, C₁₋₆haloalkyl, hydroxy, R^{a}R^{b}N—, (R^{a}R^{b}N)alkyl, (R^{a}R^{b}N)C(═O)—, and C₁₋₆alkyl optionally substituted with up to 9 halo and up to 5 hydroxy, or optionally two geminal R⁴ are together oxo;

R⁹ is selected from the group consisting of hydrogen and —C(═O)R^{9a};

R^{9a} is selected from the group consisting of —NR^{9b}R^{9c}, —OR^{9d}, C₁₋₆alkyl optionally substituted with up to 9 halo, and optionally substituted aryl;

R^{9b} is selected from the group consisting of hydrogen, C₁₋₆alkyl optionally substituted with up to 9 halo, and optionally substituted aryl;

R^{9c} is selected from the group consisting of C₁₋₆alkyl optionally substituted with up to 9 halo, and optionally substituted aryl; and

R^{9d} is selected from the group consisting of C₁₋₆alkyl optionally substituted with up to 9 halo, and optionally substituted aryl.

In some embodiments, the compound Formula XIV has the structure

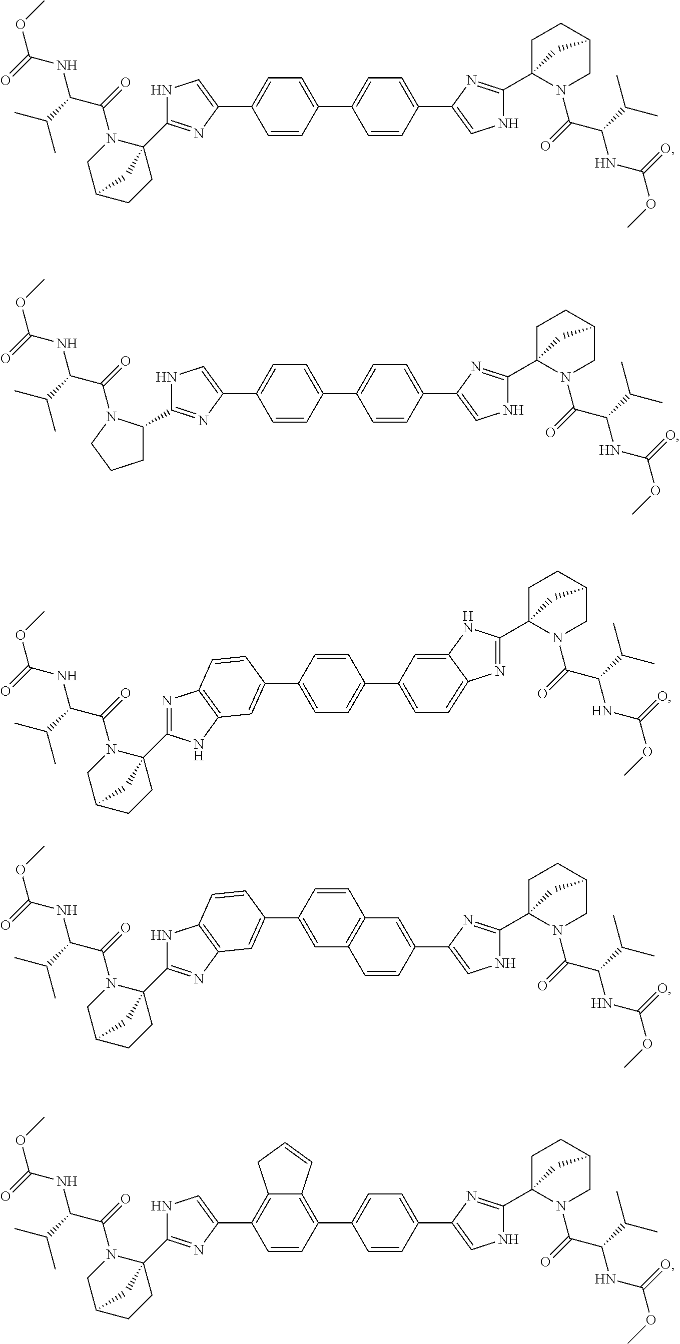

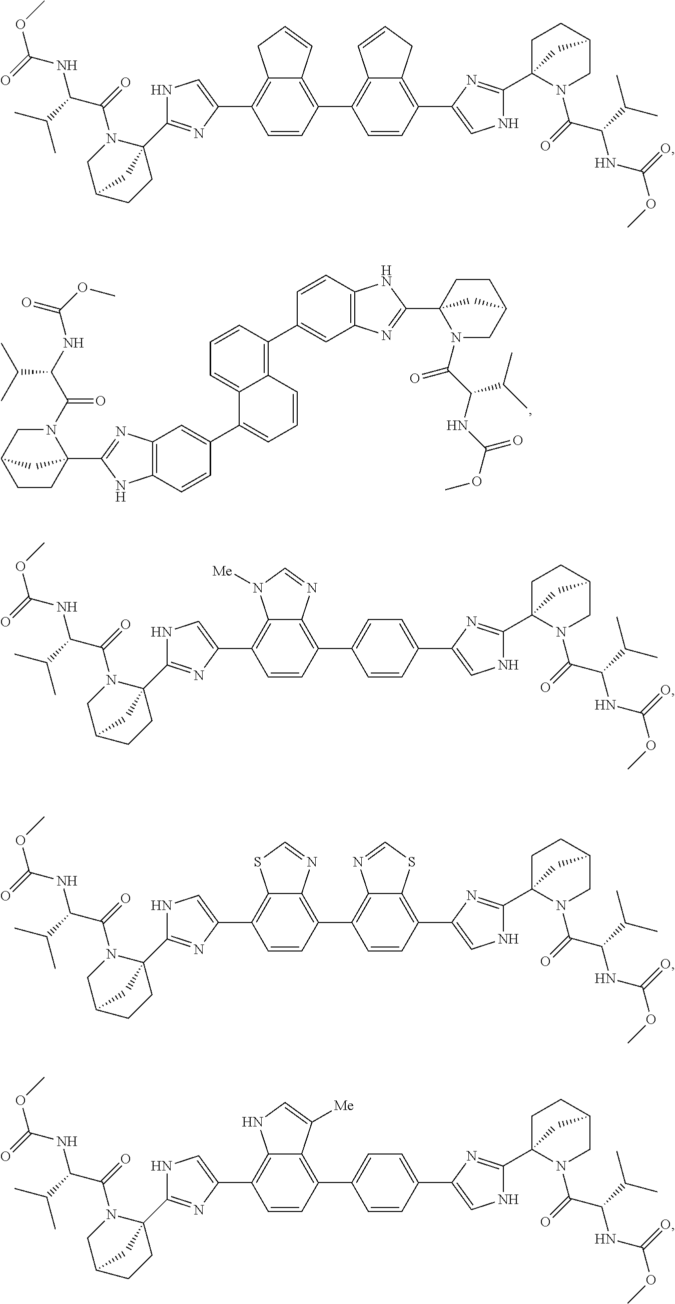

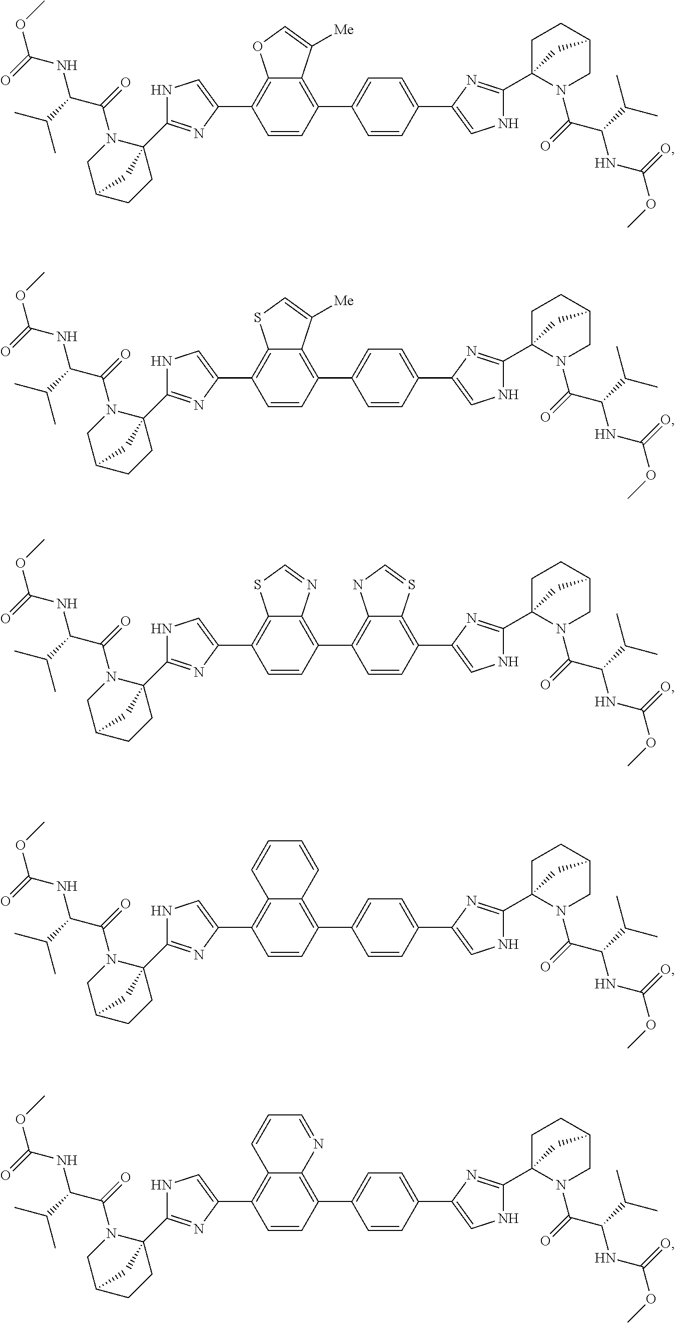

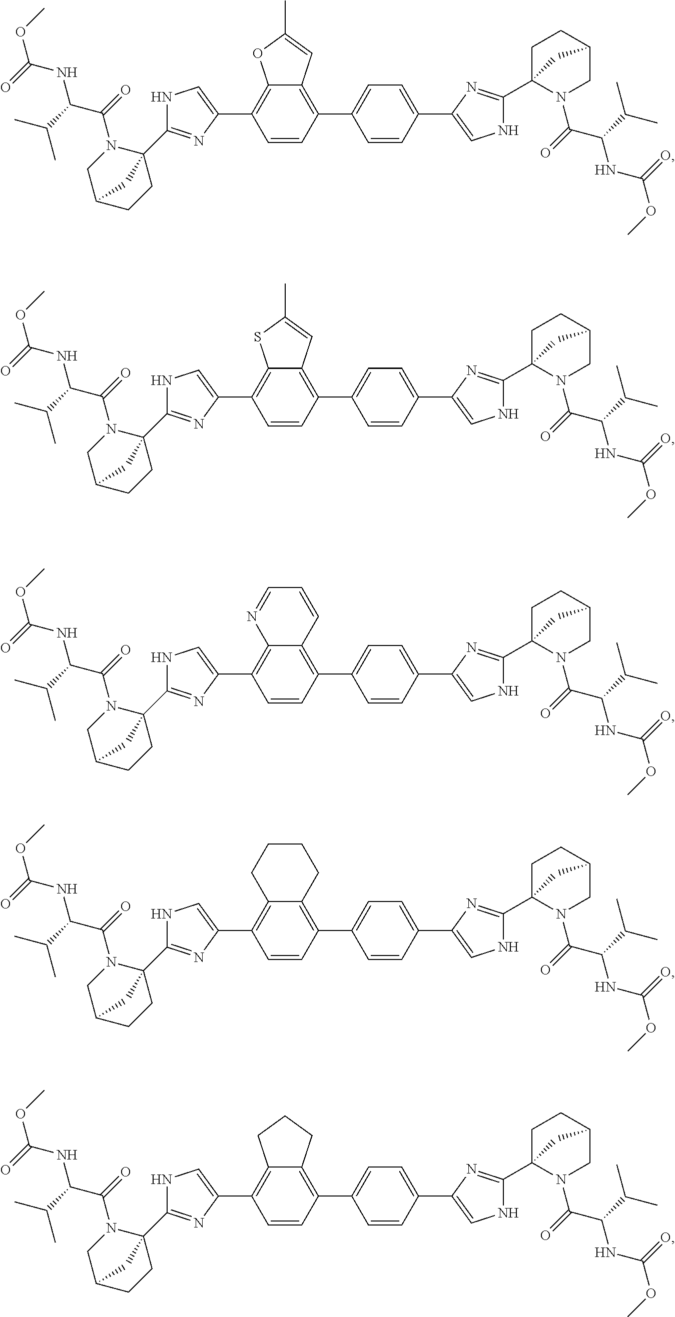

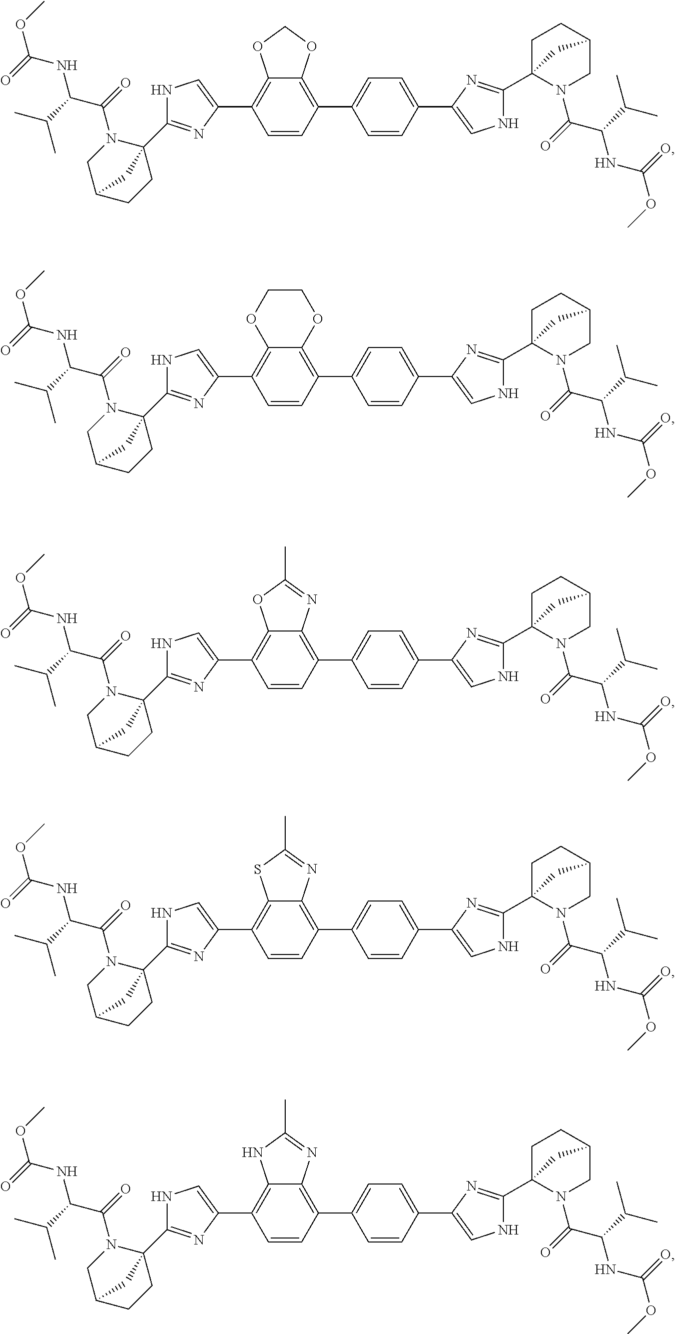

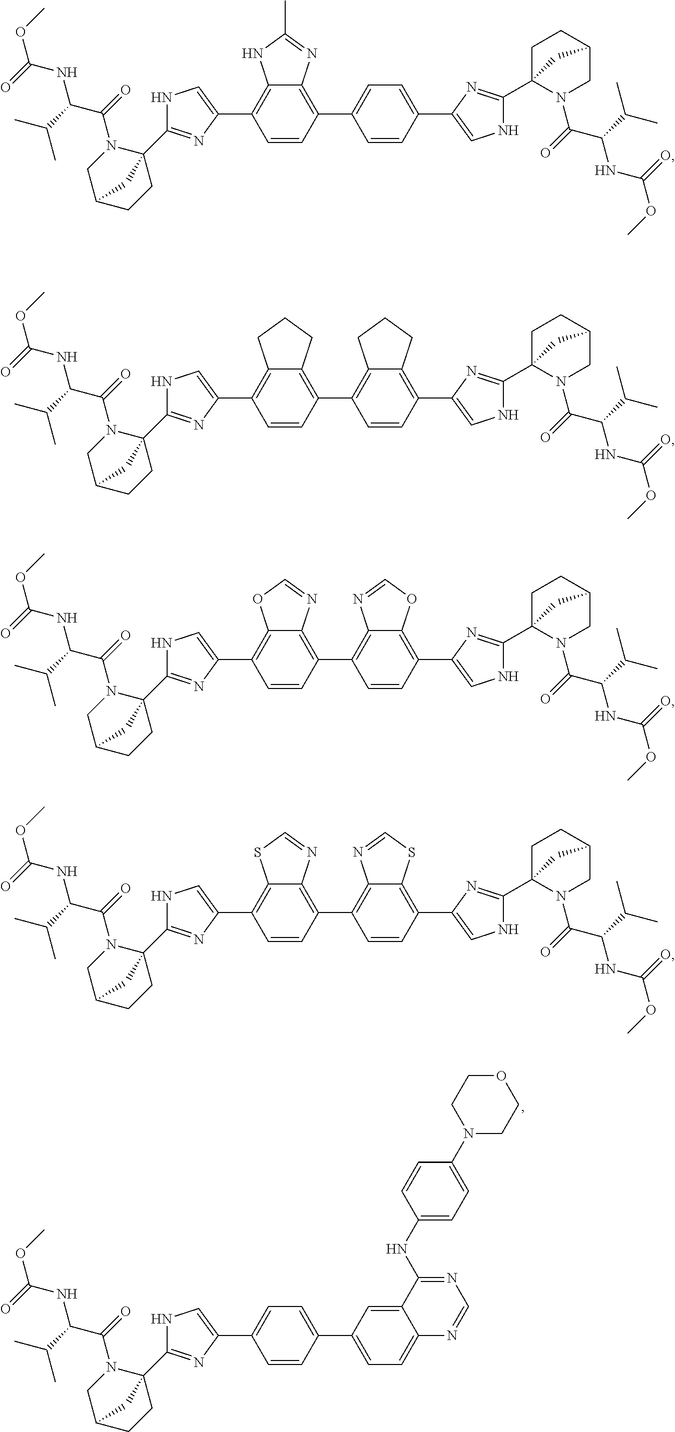

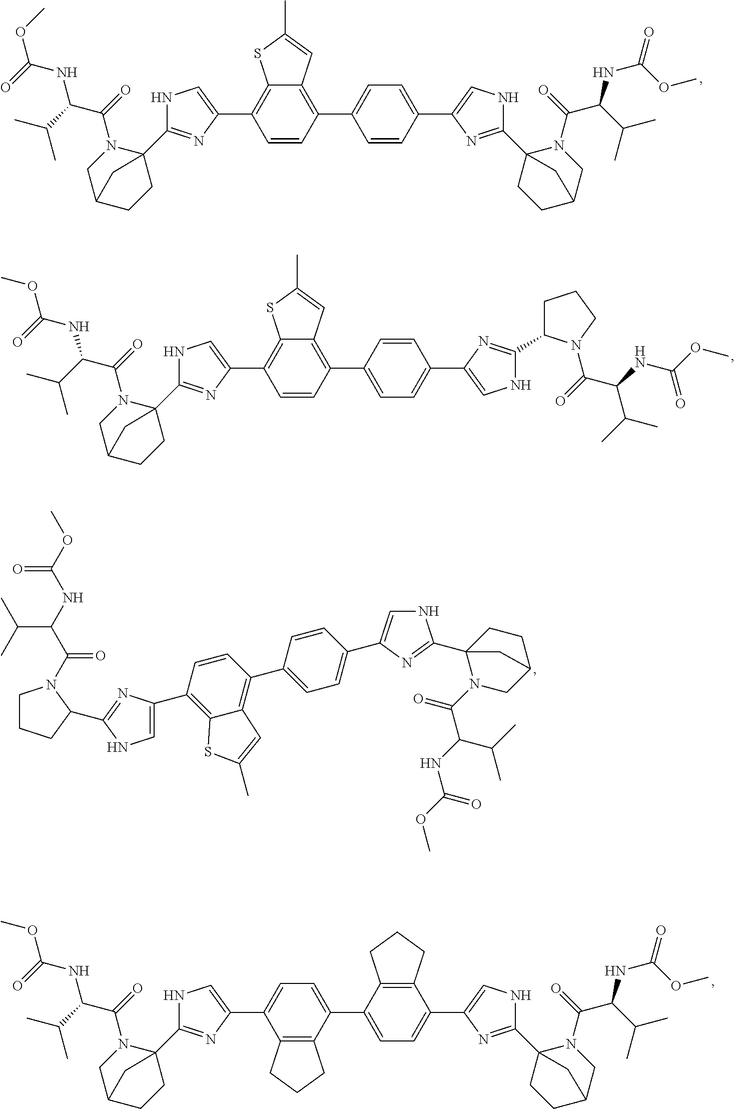

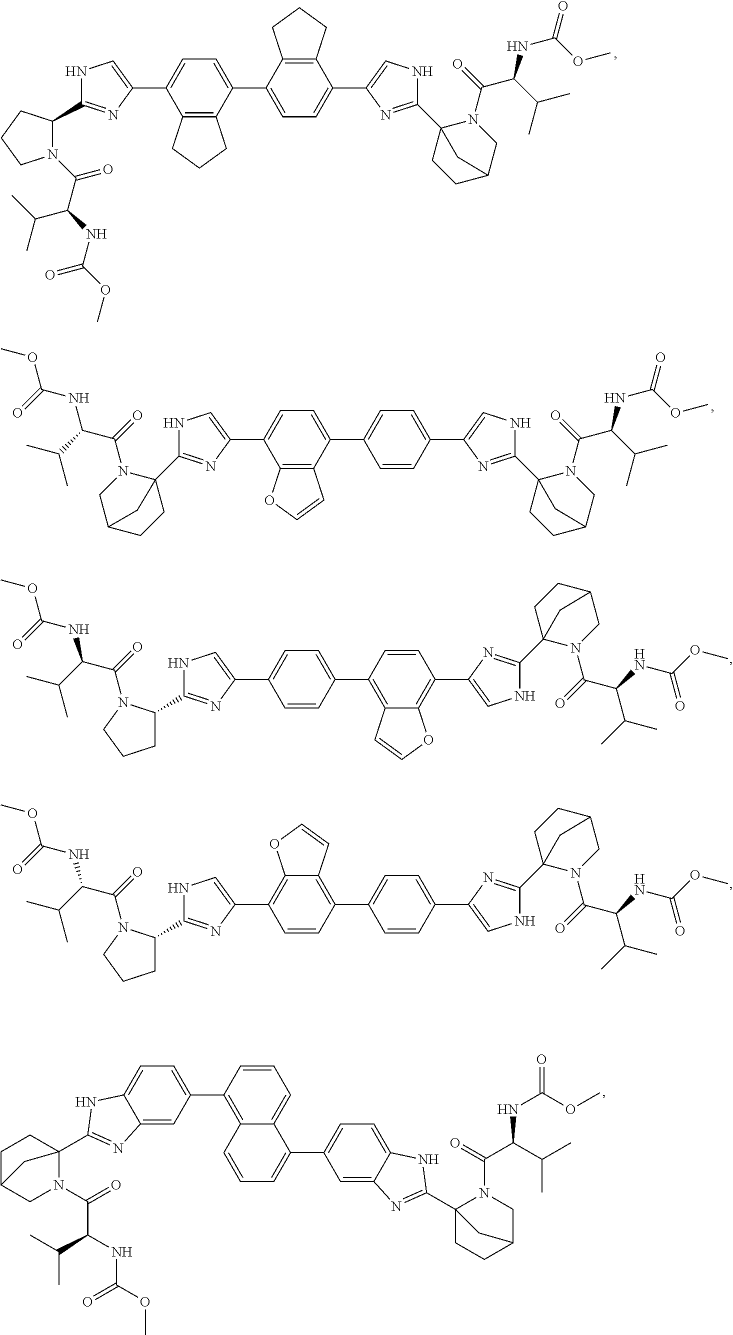

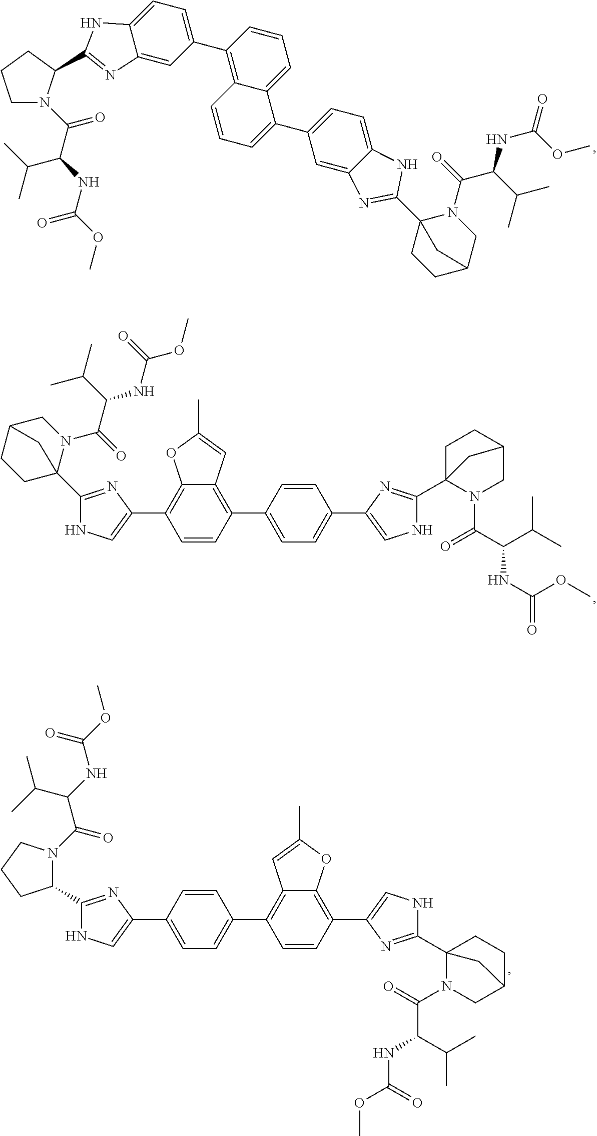

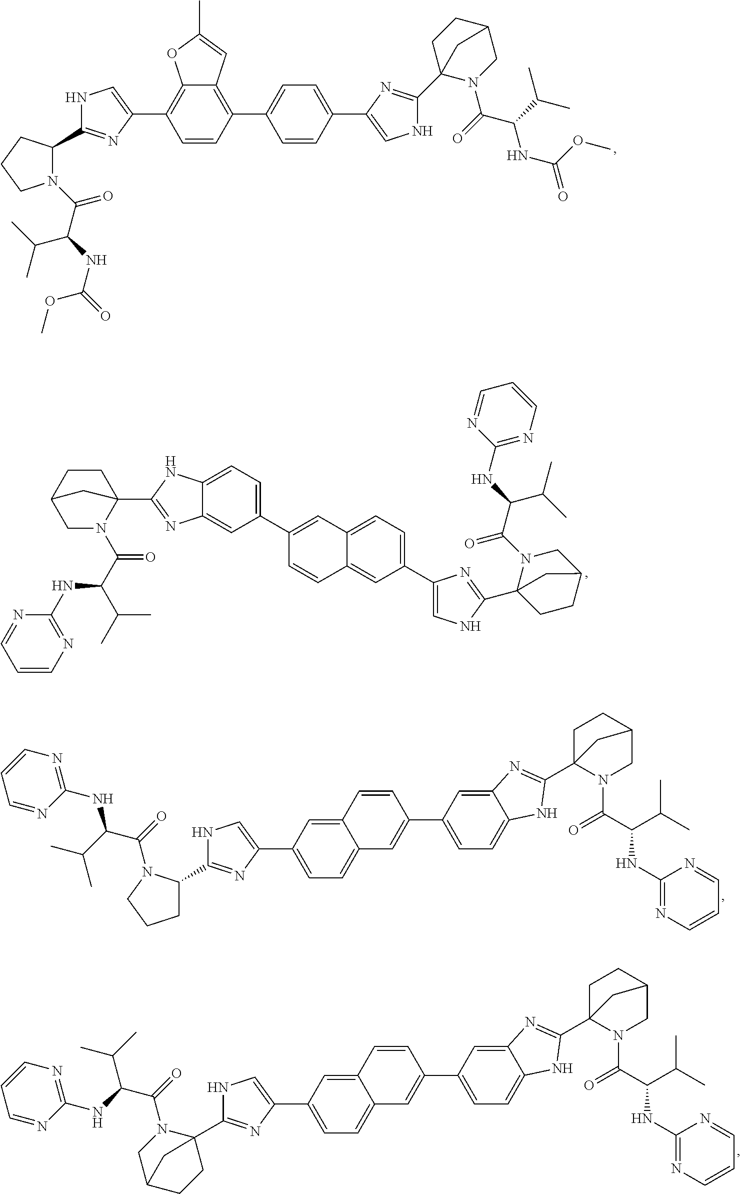

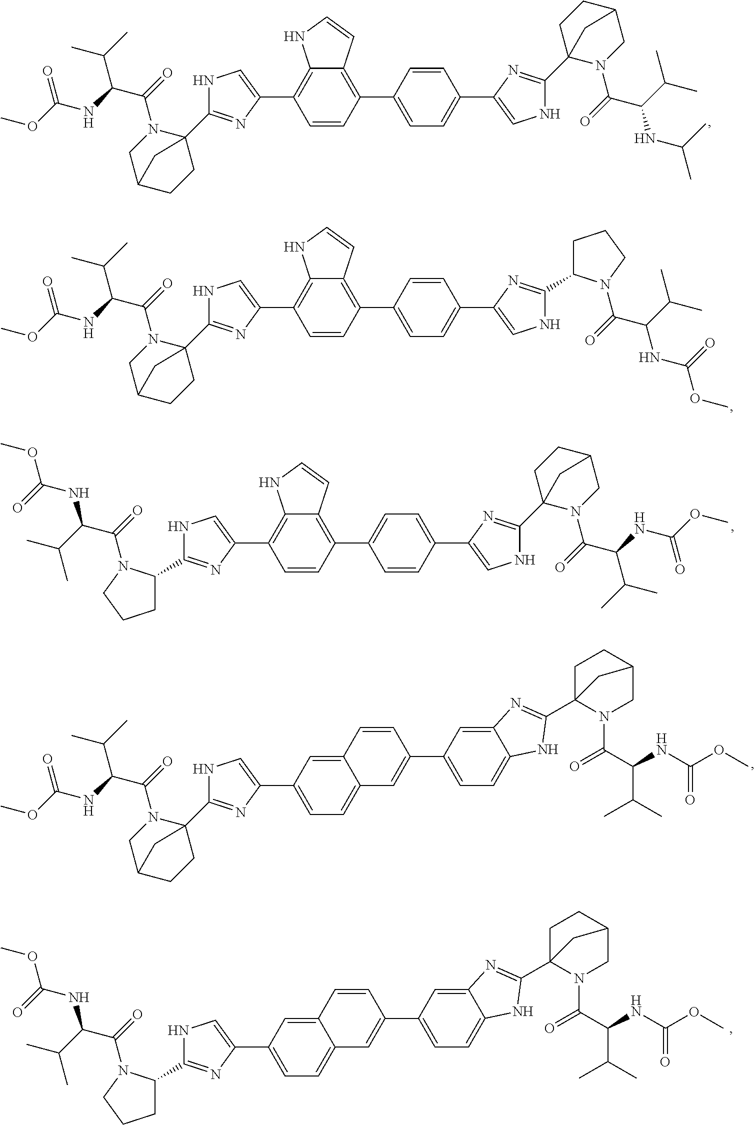

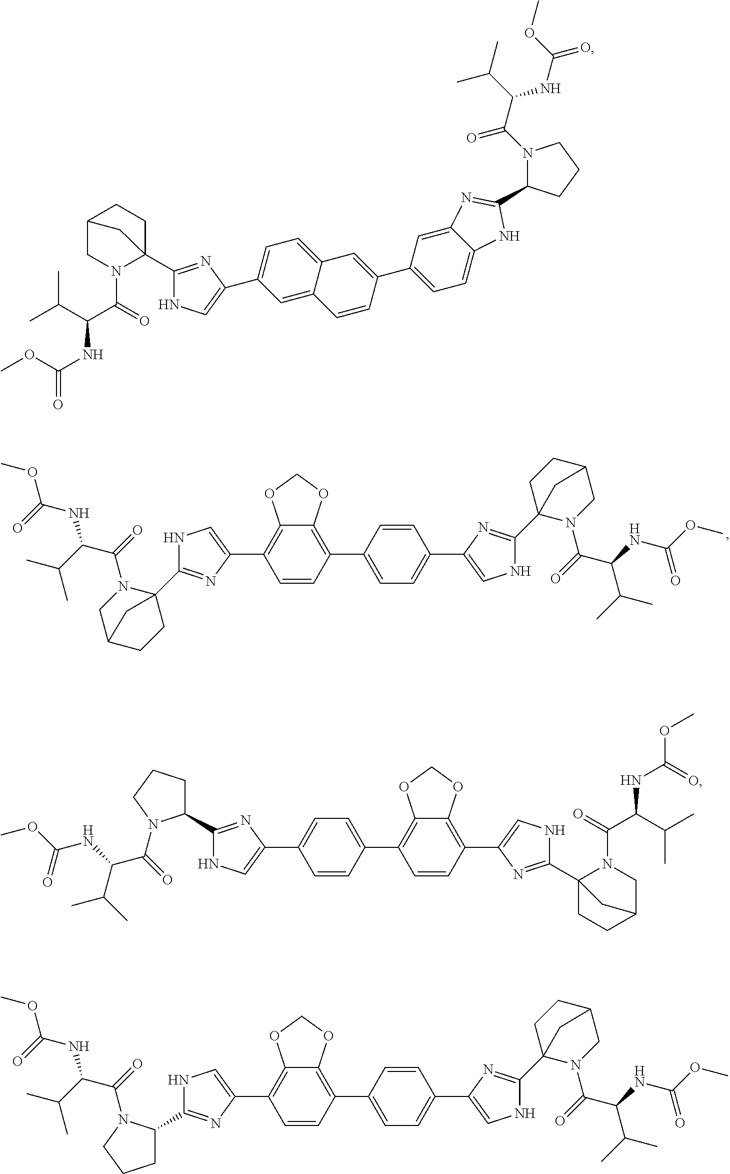

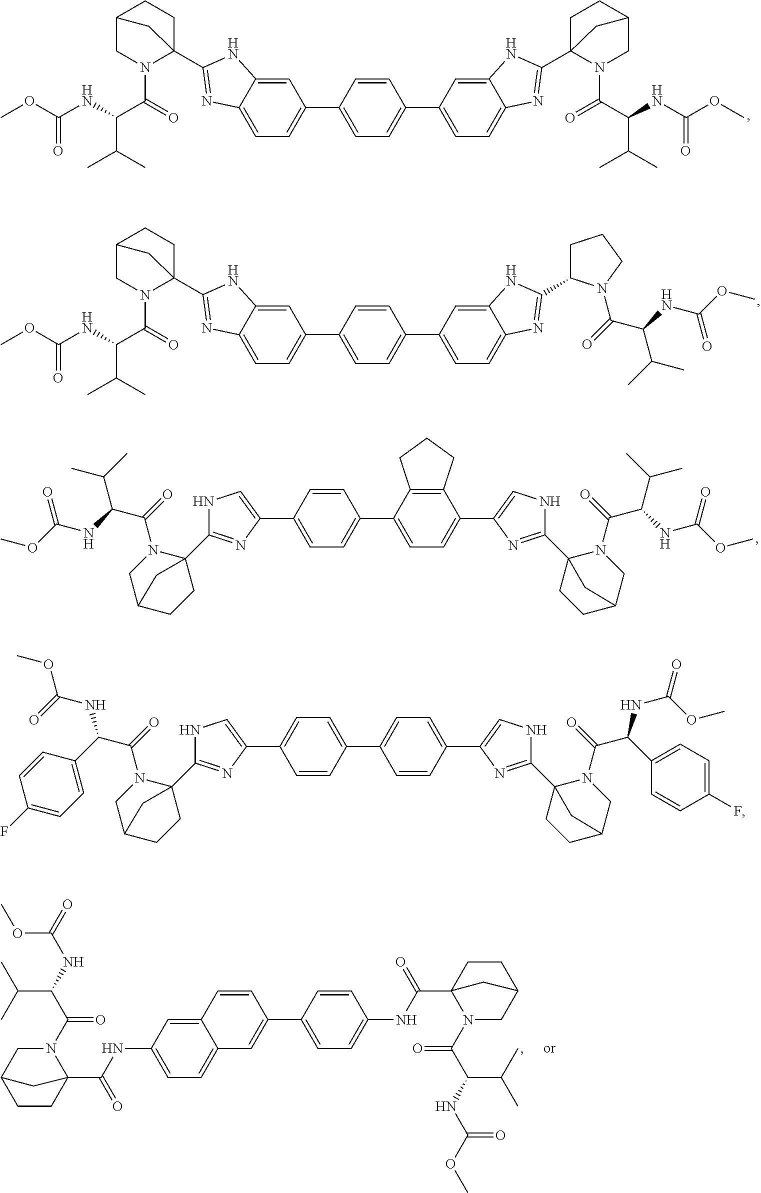

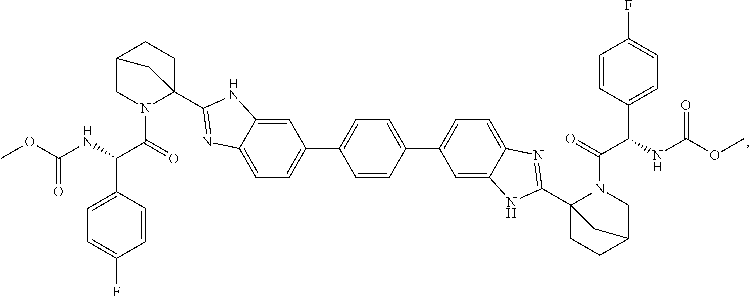

or a pharmaceutically acceptable salt thereof.

In some embodiments of Formula XIV, Q⁷ is

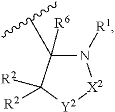

and R⁶ is C₁₋₆alkyl optionally substituted with up to 9 halo. In some embodiments, R⁶ is methyl.

In some embodiments of Formula XIV, L⁴ is selected from the group consisting of.

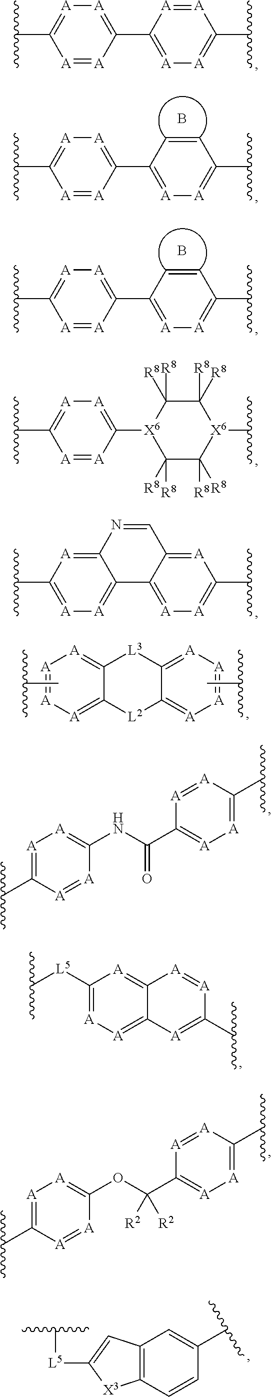

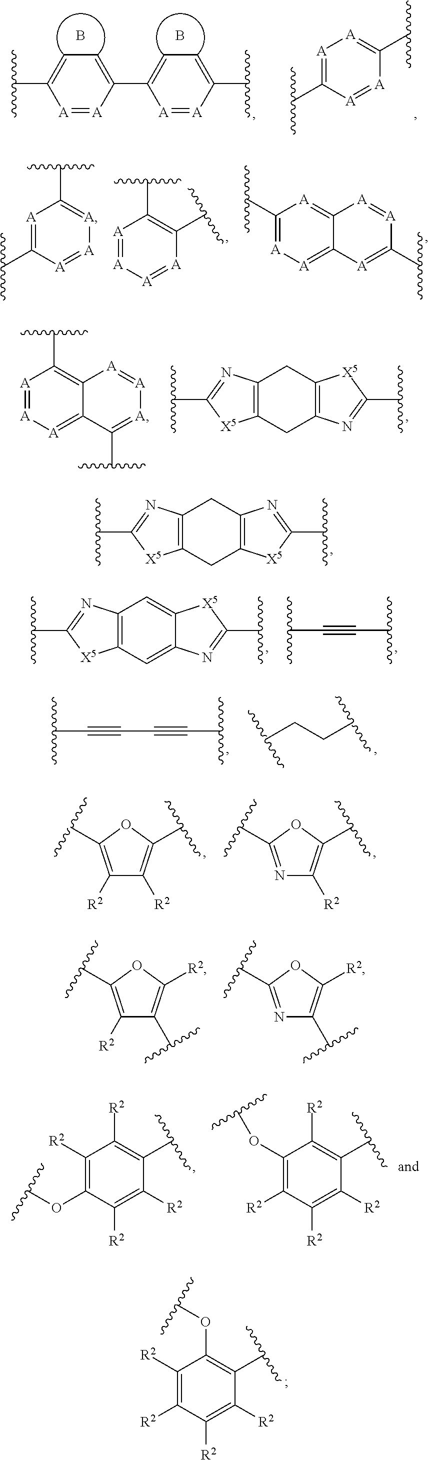

L² is selected from the group consisting of —C(═O)—, —(CH₂CH₂)—, —(CH₂O)—, —(CH₂S)—, —(CH═CH)—, —(CH═N)—, —NH—, O (oxygen), S (sulfur), and —CH₂—;

L³ is selected from the group consisting of

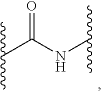

—(NR⁹)—, O (oxygen), S (sulfur), and —CH₂—;

L⁵ is selected from the group consisting of

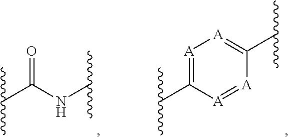

O (oxygen), NH, and —(CH═CH)—;

each X³ is separately selected from the group consisting of NH, O (oxygen), and S (sulfur);

each X⁵ is separately selected from the group consisting of —NH—, O (oxygen), S (sulfur), and —CH₂—;

each X⁶ is separately selected from the group consisting of N (nitrogen), and CR⁸;

each R⁸ is separately selected from the group consisting of hydrogen, C₁₋₆alkylOC₁₋₆alkyl, C₁₋₆alkylOC(═O)—, arylalkylOC(═O)—, halo, (R^{a}R^{b}N)alkyl, (R^{a}R^{b}N)C(═O)—, and C₁₋₆alkyl optionally substituted with up to 9 halo and up to 5 hydroxy;

each B is separately selected, wherein B is a fused optionally substituted saturated or unsaturated three- to seven-membered carbocyclic ring or a fused optionally substituted saturated or unsaturated three- to seven-membered heterocyclic ring, each optionally substituted with one or more R⁴ and;

each R⁴ is separately selected from the group consisting of C₁₋₆alkoxy, C₁₋₆alkylOC₁₋₆alkyl, C₁₋₆alkylOC(═O)—, arylalkylOC(═O)—, —COOH, halo, C₁₋₆haloalkyl, hydroxy, R^{a}R^{b}N—, (R^{a}R^{b}N)alkyl, (R^{a}R^{b}N)C(═O)—, and C₁₋₆alkyl optionally substituted with up to 9 halo and up to 5 hydroxy, or optionally two geminal R⁴ are together oxo.

Some embodiments include a compound having the structure of Formula XV:

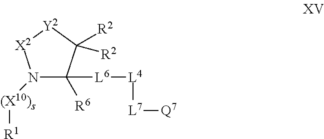

or a pharmaceutically acceptable salt thereof,

wherein:

Q⁷ is selected from the group consisting of

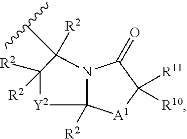

and

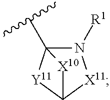

each X² is (C(R²)₂)_{q},

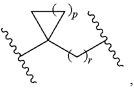

or X² is null;

each Y² is selected from O (oxygen), S (sulfur), S(O), SO₂, NR², and C(R²)₂ with the proviso that when X² is null Y² is C(R²)₂;

each X¹⁰ is (C(R²)₂)_{q};

each X¹¹ is separately selected from the group consisting of (C(R²)₂)_{q}, and

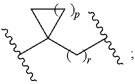

each Y¹¹ is separately selected from the group consisting of —O(C(R²)₂)ₙ—, —S(C(R²)₂)ₙ—, —S(O)(C(R²)₂)ₙ—, —SO₂(C(R²)₂)ₙ—, —NR²(C(R²)₂)ₙ—, and (C(R²)₂)_{q};

each R¹²R¹³N is separately selected, wherein R¹² and R¹³ are each separately selected from hydrogen, —[(Y¹⁴)(C(R²)₂)ᵣ(NR²)ₛ(C(R²)₂)ᵣ]—[Y¹⁴(C(R²)₂)ᵣ(NR²)ₛ(C(R²)₂)ᵣ]ₛ—(Y¹⁴)ₛ—R⁸⁰, —[(Y¹⁴)(C(R²)₂)ᵣ(NR²)ₛ(C(R²)₂)ᵣ]—Y¹⁴(C(R²)₂)ᵣO(C(R²)₂)ᵣ—(Y¹⁴)ₛ—R⁸⁰, alkoxyalkyl, alkoxyC(═O)—, C₁₋₆alkyl, C₂₋₆alkenyl, C₂₋₆alkynyl, C₃₋₇cycloalkyl, C₁₋₆alkylC(═O)—, C₃₋₇cycloalkylC(═O)—, C₁₋₆alkylsulfonyl, arylalkylOC(═O)—, aryl, arylalkyl, arylalkylC(═O)—, arylC(═O)—, arylsulfonyl, heterocyclyl, heteroaryl, heteroarylalkyl, heterocyclylalkyl, heterocyclylalkylC(═O)—, heterocyclylC(═O)—, heteroarylC(═O)—, heteroarylalkylC(═O)—, (R^{e}R^{f}N)alkyl, (R^{e}R^{f}N)alkylC(═O)—, and (R^{e}R^{f}N)C(═O)—,
said alkoxyalkyl, alkoxyC(═O)—, C₁₋₆alkyl, C₃₋₇cycloalkyl, C₁₋₆alkylC(═O)—, C₃₋₇cycloalkylC(═O)—, C₁₋₆alkylsulfonyl, arylalkylOC(═O)—, aryl, arylalkyl, arylalkylC(═O)—, arylC(═O)—, arylsulfonyl, heterocyclyl, heteroaryl, heteroarylalkyl, heterocyclylalkyl, heterocyclylalkylC(═O)—, heterocyclylC(═O)—, heteroarylC(═O)—, heteroarylalkylC(═O)—, and alkyl in (R^{e}R^{f}N)alkyl and (R^{e}R^{f}N)alkylC(═O)— are each optionally substituted with one or more R^{1ab};

or R¹²R¹³N is a heterocyclyl linked through a ring nitrogen atom optionally substituted with one or more of oxo, —[(Y¹⁴)(C(R²)₂)ᵣ(NR²)ₛ(C(R²)₂)ᵣ]—[Y¹⁴(C(R²)₂)ᵣ(NR²)ₛ(C(R²)₂)ᵣ]ₛ—(Y¹⁴)ₛ—R⁸⁰, —[(Y¹⁴)(C(R²)₂)ᵣ(NR²)ₛ(C(R²)₂)ᵣ]—Y¹⁴(C(R²)₂)ᵣO(C(R²)₂)ᵣ—(Y¹⁴)ₛ—R⁸⁰, alkoxyalkyl, alkoxyC(═O)—, C₁₋₆alkyl, C₂₋₆alkenyl, C₂₋₆alkynyl, C₃₋₇cycloalkyl, C₁₋₆alkylC(═O)—, C₃₋₇cycloalkylC(═O)—, C₁₋₆alkylsulfonyl, arylalkylOC(═O)—, aryl, arylalkyl, arylalkylC(═O)—, arylC(═O)—, arylsulfonyl, heterocyclyl, heteroaryl, heteroarylalkyl, heterocyclylalkyl, heterocyclylalkylC(═O)—, heterocyclylC(═O)—, heteroarylC(═O)—, heteroarylalkylC(═O)—, (R^{e}R^{f}N)alkyl, (R^{e}R^{f}N)alkylC(═O)—, and (R^{e}R^{f}N)C(═O)—,
said alkoxyalkyl, alkoxyC(═O)—, C₁₋₆alkyl, C₃₋₇cycloalkyl, C₁₋₆alkylC(═O)—, C₃₋₇cycloalkylC(═O)—, C₁₋₆alkylsulfonyl, arylalkylOC(═O)—, aryl, arylalkyl, arylalkylC(═O)—, arylC(═O)—, arylsulfonyl, heterocyclyl, heteroaryl, heteroarylalkyl, heterocyclylalkyl, heterocyclylalkylC(═O)—, heterocyclylC(═O)—, heteroarylC(═O)—, heteroarylalkylC(═O)—, and alkyl in (R^{e}R^{f}N)alkyl and (R^{e}R^{f}N)alkylC(═O)— are each optionally substituted with one or more R^{1ab};

each R^{1ab} is separately selected from the group consisting of —[(Y¹⁴)(C(R²)₂)ᵣ(NR²)ₛ(C(R²)₂)ᵣ]—[Y¹⁴(C(R²)₂)ᵣ(NR²)ₛ(C(R²)₂)ᵣ]ₛ—(Y¹⁴)ₛ—R⁸⁰, —[(Y¹⁴)(C(R²)₂)ᵣ(NR²)ₛ(C(R²)₂)ᵣ]—Y¹⁴(C(R²)₂)ᵣO(C(R²)₂)ᵣ—(Y¹⁴)ₛ—R⁸⁰, —C(R^{2a})₂NR^{3a}R^{3b}, alkoxyalkyl, C₁₋₆alkylOC(═O)—, C₁₋₆alkylOC(═O)C₁₋₆alkyl, C₁₋₆alkylC(═O)C₁₋₆alkyl, aryl, aryl(CH₂)ₙ—, aryl(CH₂)ₙO—, aryl(CH═CH)ₘ—, arylalkylO—, arylalkyl, arylOalkyl, cycloalkyl, (cycloalkyl)(CH═CH)ₘ—, (cycloalkyl)alkyl, cycloalkylOalkyl, heterocyclyl, heterocyclyl(CH═CH)ₘ—, heterocyclylalkoxy, heterocyclylalkyl, heterocyclylOalkyl, hydroxyalkyl, R^{c}R^{d}N—, R^{e}R^{d}N(CH₂)ₙ—, (R^{c}R^{d}N)(CH═CH)ₘ—, (R^{c}R^{d}N)alkyl, (R^{c}R^{d}N)C(═O)—, C₁₋₆alkoxy optionally substituted with up to 9 halo, and C₁₋₆alkyl optionally substituted with up to 9 halo, said aryl and heteroaryl each optionally substituted with cyano, halo, nitro, hydroxyl, C₁₋₆alkoxy optionally substituted with up to 9 halo, and C₁₋₆alkyl optionally substituted with up to 9 halo;

each R⁸⁰ is separately selected from the group consisting of hydrogen, alkoxyalkyl, C₁₋₆alkyl, C₃₋₇cycloalkyl, aryl, arylalkyl, heterocyclyl, heterocyclylalkyl, heteroaryl, heteroarylalkyl, heterocyclylalkyl, and (R^{e}R^{f}N)alkyl, said alkoxyalkyl, C₁₋₆alkyl, C₃₋₇cycloalkyl, aryl, arylalkyl, heterocyclyl, heterocyclylalkyl, heteroaryl, heteroarylalkyl, heterocyclylalkyl, and alkyl in (R^{e}R^{f}N)alkyl are each optionally substituted with one or more R^{1ac};

each R^{1ac} is separately selected from the group consisting of —C(R^{2a})₂NR^{3a}R^{3b}, alkoxyalkyl, C₁₋₆alkylOC(═O)—, C₁₋₆alkylOC(═O)C₁₋₆alkyl, C₁₋₆alkylC(═O)C₁₋₆alkyl, aryl, aryl(CH₂)ₙ—, aryl(CH₂)ₙO—, aryl(CH═CH)ₘ—, arylalkylO—, arylalkyl, arylOalkyl, cycloalkyl, (cycloalkyl)(CH═CH)ₘ—, (cycloalkyl)alkyl, cycloalkylOalkyl, heterocyclyl, heterocyclyl(CH═CH)ₘ—, heterocyclylalkoxy, heterocyclylalkyl, heterocyclylOalkyl, hydroxyalkyl, R^{c}R^{d}N—, R^{c}R^{d}N(CH₂)ₙ—, (R^{c}R^{d}N)(CH═CH)ₘ—, (R^{c}R^{d}N)alkyl, (R^{c}R^{d}N)C(═O)—, C₁₋₆alkoxy optionally substituted with up to 9 halo, and C₁₋₆alkyl optionally substituted with up to 9 halo, said aryl and heteroaryl each optionally substituted with cyano, halo, nitro, hydroxyl, C₁₋₆alkoxy optionally substituted with up to 9 halo, and C₁₋₆alkyl optionally substituted with up to 9 halo;

each Y¹⁴ is separately selected from the group consisting of —C(═O)—, —S(═O)—, —C(═S)—, —S(═O)₂—, —C(═O)O—, —C(═O)NR^{2c}—, —S(═O)₂NR^{2c}—, —C(═O)NR^{2c}C(═O)—, and —C(CF₃)₂NR^{2c}—,

R¹ is selected from the group consisting of R^{1aa}R^{1a}C(═O)— and R^{1a}C(═S)—;

each R^{1a} is separately selected from the group consisting of —C(R^{2a})₂NR^{3a}R^{3b}, alkoxyalkyl, C₁₋₆alkylOC(═O)—, C₁₋₆alkylOC(═O)C₁₋₆alkyl, C₁₋₆alkylC(═O)C₁₋₆alkyl, aryl, aryl(CH₂)ₙ—, aryl(CH₂)ₙO—, aryl(CH═CH)ₘ—, arylalkylO—, arylalkyl, arylOalkyl, cycloalkyl, (cycloalkyl)(CH═CH)ₘ—, (cycloalkyl)alkyl, cycloalkylOalkyl, heterocyclyl, heterocyclyl(CH═CH)ₘ—, heterocyclylalkoxy, heterocyclylalkyl, heterocyclylOalkyl, hydroxyalkyl, R^{c}R^{d}N—, R^{c}R^{d}N(CH₂)ₙ—, (R^{c}R^{d}N)(CH═CH)ₘ—, (R^{c}R^{d}N)alkyl, (R^{c}R^{d}N)C(═O)—, C₁₋₆alkoxy optionally substituted with up to 9 halo, and C₁₋₆alkyl optionally substituted with up to 9 halo, said aryl and heteroaryl each optionally substituted with cyano, halo, nitro, hydroxyl, C₁₋₆alkoxy optionally substituted with up to 9 halo, and C₁₋₆alkyl optionally substituted with up to 9 halo;

R^{1aa} is selected from the group consisting of —C(R^{2a})₂NR^{3a}R^{3b}, —[(Y¹⁴)(C(R²)₂)ᵣ(NR²)ₛ(C(R²)₂)ᵣ]—[Y¹⁴(C(R²)₂)ᵣ(NR²)ₛ(C(R²)₂)ᵣ]ₛ—(Y¹⁴)ₛ—R⁸⁰, —[(Y¹⁴)(C(R²)₂)ᵣ(NR²)ₛ(C(R²)₂)ᵣ]—Y¹⁴(C(R²)₂)ᵣO(C(R²)₂)ᵣ—(Y¹⁴)ₛ—R⁸⁰, alkoxyalkyl, alkoxyC(═O)—, C₁₋₆alkyl, C₂₋₆alkenyl, C₂₋₆alkynyl, C₃₋₇cycloalkyl, C₁₋₆alkylC(═O)—, C₃₋₇cycloalkylC(═O)—, C₁₋₆alkylsulfonyl, arylalkylOC(═O)—, aryl, arylalkyl, arylalkylC(═O)—, arylC(═O)—, arylsulfonyl, heterocyclyl, heteroaryl, heteroarylalkyl, heterocyclylalkyl, heterocyclylalkylC(═O)—, heterocyclylC(═O)—, heteroarylC(═O)—, heteroarylalkylC(═O)—, (R^{e}R^{f}N)alkyl, (R^{e}R^{f}N)alkylC(═O)—, and (R^{e}R^{f}N)C(═O)—,
said alkoxyalkyl, alkoxyC(═O)—, C₁₋₆alkyl, C₃₋₇cycloalkyl, C₁₋₆alkylC(═O)—, C₃₋₇cycloalkylC(═O)—, C₁₋₆alkylsulfonyl, arylalkylOC(═O)—, aryl, arylalkyl, arylalkylC(═O)—, arylC(═O)—, arylsulfonyl, heterocyclyl, heteroaryl, heteroarylalkyl, heterocyclylalkyl, heterocyclylalkylC(═O)—, heterocyclylC(═O)—, heteroarylC(═O)—, heteroarylalkylC(═O)—, and alkyl in (R^{e}R^{f}N)alkyl and (R^{e}R^{f}N)alkylC(═O)— are each optionally substituted with one or more R^{1ab};

each R¹⁰ is R^{c}R^{d}N—;

each R¹¹ is separately selected from the group consisting of H (hydrogen), alkoxyalkyl, C₁₋₆alkylOC(═O)C₁₋₆alkyl, C₁₋₆alkylC(═O)C₁₋₆alkyl, aryl(CH₂)ₙ—, arylalkyl, arylOalkyl, cycloalkyl, (cycloalkyl)alkyl, cycloalkylOalkyl, heterocyclylalkyl, heterocyclylOalkyl, hydroxyalkyl, R^{c}R^{d}N(CH₂)ₙ—, (R^{c}R^{d}N)alkyl, and C₁₋₆alkyl optionally substituted with up to 9 halo;

each R^{c}R^{d}N is separately selected, wherein R^{c} and R^{d} are each separately selected from hydrogen, alkoxyC(═O)—, C₁₋₆alkyl, C₁₋₆alkylC(═O)—, C₁₋₆alkylsulfonyl, arylalkylOC(═O)—, arylalkyl, arylalkylC(═O)—, arylC(═O)—, arylsulfonyl, heterocyclylalkyl, heterocyclylalkylC(═O)—, heterocyclylC(═O)—, (R^{e}R^{f}N)alkyl, (R^{e}R^{f}N)alkylC(═O)—, and (R^{e}R^{f}N)C(═O)—, wherein the alkyl part of arylalkyl, arylalkylC(═O)—, heterocyclylalkyl, and heterocyclylalkylC(═O)— are each optionally substituted with one R^{e}R^{f}N— group; and wherein the aryl part of arylalkyl, arylalkylC(═O)—, arylC(═O)—, and arylsulfonyl, and the heterocyclyl part of heterocyclylalkyl, heterocyclylalkylC(═O)—, and heterocyclylC(═O)— are each optionally substituted with up to three substituents each independently selected from the group consisting of cyano, halo, nitro, C₁₋₆alkoxy optionally substituted with up to 9 halo, and C₁₋₆alkyl optionally substituted with up to 9 halo;

each R^{e}R^{f}N is separately selected, wherein R^{e} and R^{f} are each separately selected from hydrogen, C₁₋₆alkyl, aryl, arylalkyl, cycloalkyl, (cyclolalkyl)alkyl, heterocyclyl, heterocyclylalkyl, (R^{x}R^{y}N)alkyl, and (R^{x}R^{y}N)C(═O)—;

each R^{x}R^{y}N is separately selected, wherein R^{x} and R^{y} are each separately selected from hydrogen, C₁₋₆alkylOC(═O)—, C₁₋₆alkyl, C₁₋₆alkylC(═O)—, aryl, arylalkyl, cycloalkyl, and heterocyclyl;

each C(R^{2a})₂ is separately selected, wherein each R^{2a} is separately selected from the group consisting of hydrogen, C₁₋₆alkyl optionally substituted with up to 9 halo, aryl(CH₂)ₙ—, and heteroaryl(CH₂)ₙ—, said aryl and heteroaryl each optionally substituted with cyano, halo, nitro, hydroxyl, C₁₋₆alkoxy optionally substituted with up to 9 halo, and C₁₋₆alkyl optionally substituted with up to 9 halo, or optionally C(R^{2a})₂ is

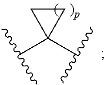

each R^{3a} is separately selected from the group consisting of hydrogen, and optionally substituted C₁₋₆alkyl;

each R^{3b} is separately selected from the group consisting of optionally substituted C₁₋₆alkyl, heteroaryl, —(CH₂)ₙC(═O)NR^{4a}R^{4b}, —(CH₂)ₙC(═O)OR^{5a}, and —(CH₂)ₙC(═O)R^{6a} said heteroaryl optionally substituted with cyano, halo, nitro, hydroxyl, C₁₋₆alkoxy optionally substituted with up to 9 halo, and C₁₋₆alkyl optionally substituted with up to 9 halo;

each R^{4a}R^{4b}N is separately selected, wherein R^{4a} and R^{4b} are each separately selected from the group consisting of hydrogen, optionally substituted C₁₋₆alkyl, and aryl(CH₂)ₙ—;

each R^{5a} is separately selected from the group consisting of optionally substituted C₁₋₆alkyl, and aryl(CH₂)ₙ—;

each R^{6a} is separately selected from the group consisting of optionally substituted C₁₋₆alkyl, and aryl(CH₂)ₙ—;

each A¹ is separately selected from the group consisting of C₂₋₆alkenyl, C₁₋₆alkyl, and —(CH₂)ₙ—O—(CH₂)ₘ—, each optionally substituted with one or more R²;

each R² is separately selected, wherein R² is selected from the group consisting of hydrogen, halo, hydroxy, C₁₋₆alkoxy, C₁₋₆alkyl, C₂₋₆alkenyl, C₂₋₆alkynyl, alkyoxyalkyl, C₃₋₇cycloalkyl, aryl, arylalkyl, heteroaryl, heteroarylalkyl, heterocyclyl, heterocyclylalkyl, (R^{e}R^{f}N)alkyl, R^{a}R^{b}N—, said C₁₋₆alkyl optionally substituted with one or more halo, —OR^{2b}, —C(═O)OR^{2b}, —C(═O)NHR^{2b}, —NHC(═NH)NHR^{2b}, —NHR^{2b}, SR^{2b}, imidazolyl, indolyl, —SCH₃, phenyl, and 4-hydroxyphenyl, said C₁₋₆alkoxy, alkoxyalkyl, aryl, C₂₋₆alkenyl, C₂₋₆alkynyl, C₃₋₇cycloalkyl, arylalkyl, heterocyclyl, heteroaryl, heteroarylalkyl, heterocyclylalkyl, and alkyl in (R^{e}R^{f}N)alkyl each optionally substituted with one or more R⁴, or optionally two vicinal R² and the carbons to which they are attached are together a fused three- to six-membered carbocyclic ring optionally substituted with up to two C₁₋₆alkyl groups;

R^{2b} is selected from the group consisting of hydrogen, C₁₋₆alkyl, C₂₋₆alkenyl, C₂₋₆alkynyl, C₃₋₇cycloalkyl, aryl, arylalkyl, heteroaryl, heteroarylalkyl, heterocyclyl, and heterocyclylalkyl;

each R^{2c} is selected from the group consisting of hydrogen, C₁₋₆alkyl, C₂₋₆alkenyl, C₂₋₆alkynyl, C₃₋₇cycloalkyl, aryl, arylalkyl, heteroaryl, heteroarylalkyl, heterocyclyl, and heterocyclylalkyl, said alkyl optionally substituted with R^{e}R^{f}N—, alkoxy, or C₁₋₆alkylS—;

each R^{a}R^{b}N is separately selected, wherein R^{a} and R^{b} are each separately selected from the group consisting of hydrogen, C₂₋₆alkenyl, and C₁₋₆alkyl;

L⁴ is selected from the group consisting of -(J²)ₛ-(L⁵)ₛ-(J²)ₛ-(L⁵)ₛ-J²-,

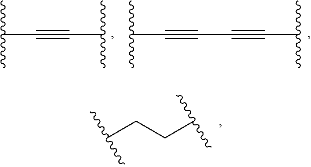

—C(═O)—, O (oxygen), —OC(R²)₂—,

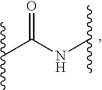

### —C(CF₃)₂NR^{2c}—, NH, and —(CH═CH)—;

each J² is separately selected from the group consisting of aryl, heteroaryl, heterocyclyl, cycloalkyl, cycloalkenyl, and polycyclic hydrocarbon, each optionally substituted with one or more R¹⁵;

each R¹⁴ is separately selected from the group consisting of hydroxy, C₁₋₆alkoxy optionally substituted with up to 9 fluoro, C₁₋₆alkylOC₁₋₆alkyl, C₁₋₆alkylOC(═O)—, arylalkylOC(═O)—, —COOH, halo, and C₁₋₆alkyl optionally substituted with up to 9 halo;

each R¹⁵ is separately selected from the group consisting of halo, hydroxy, C₁₋₆alkylOC₁₋₆alkyl, C₁₋₆alkylOC(═O)—, arylalkylOC(═O)—, —COOH, R⁹, R^{x}R^{y}N, R^{x}R^{Y}NC(═O), R^{x}R^{y}NC₁₋₆alkyl, heteroaryl, aryl, C₁₋₆alkyl optionally substituted with up to 9 halo, C₁₋₆alkoxy optionally substituted with up to 9 halo, C₁₋₆haloalkyl, R^{a}R^{b}N—, (R^{a}R^{b}N)alkyl, (R^{a}R^{b}N)C(═O)—, and C₁₋₆alkyl substituted with up to 5 hydroxy, said substituent aryl and heteroaryl are each optionally substituted with one or more R¹⁴, or optionally two vicinal R¹⁵ and the carbons to which they are attached are together a fused three- to six-membered carbocyclic ring optionally substituted with up to two C₁₋₆alkyl groups, or optionally two geminal R¹⁵ and the carbon to which they are attached are together a three- to six-membered carbocyclic ring optionally substituted with up to two C₁₋₆alkyl groups, or optionally two geminal R¹⁵ are together oxo;

each L⁵ is separately selected from the group consisting of

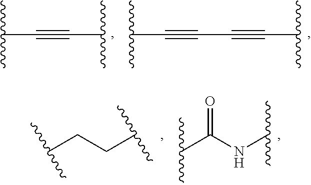

### —C(CF₃)₂NR^{2c}—,

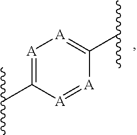

—C(R²)₂, —C(R²)₂O—, —C(═O)—, O (oxygen), NH, and —(CH═CH)—;

each A is separately selected from the group consisting of CR³ and N (nitrogen);

L⁶ is selected from the group consisting of

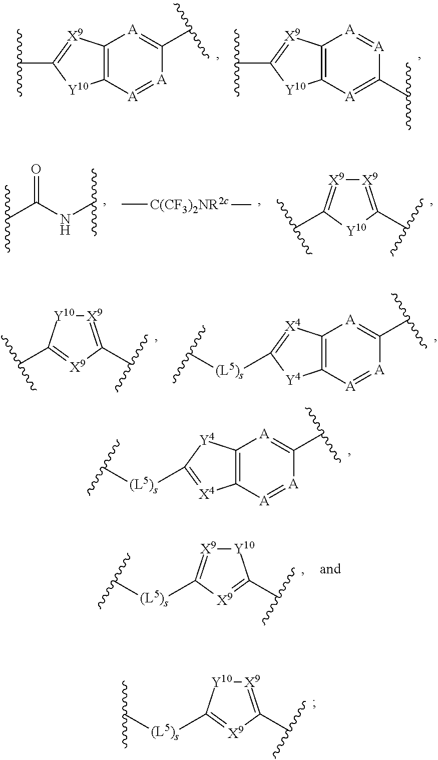

L⁷ is selected from the group consisting of

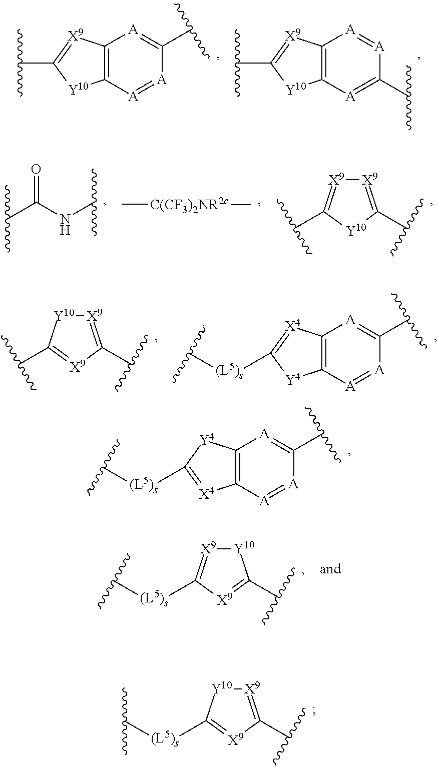

each X⁴ is separately selected from the group consisting of CR⁴ and N (nitrogen);

each Y⁴ is separately selected from the group consisting of C(R⁴)₂, NR⁴, O (oxygen), and S (sulfur);

each X⁹ is separately selected from the group consisting of CH and N (nitrogen);

each Y¹⁰ is separately selected from the group consisting of —CH₂— and —NH—;

each m separately is 1 or 2;

each n separately is 0, 1 or 2;

each p separately is 1, 2, 3 or 4;

each q separately is 1, 2, 3, 4 or 5;

each r separately is 0, 1, 2, 3, or 4;

each s separately is 0 or 1;

each R³ is separately selected from the group consisting of hydrogen, C₁₋₆alkoxy, C₁₋₆alkylOC₁₋₆alkyl, C₁₋₆alkylOC(═O)—, arylalkylOC(═O)—, —COOH, halo, hydroxy, R^{a}R^{b}N—, (R^{a}R^{b}N)alkyl, (R^{a}R^{b}N)C(═O)—, and C₁₋₆alkyl optionally substituted with up to 9 halo and up to 5 hydroxy;

each R⁴ is separately selected from the group consisting of H (hydrogen), C₁₋₆alkoxy, C₁₋₆alkylOC₁₋₆alkyl, C₁₋₆alkylOC(═O)—, arylalkylOC(═O)—, —COOH, halo, C₁₋₆haloalkyl, hydroxy, R^{a}R^{b}N—, (R^{a}R^{b}N)alkyl, (R^{a}R^{b}N)C(═O)—, and C₁₋₆alkyl optionally substituted with up to 9 halo and up to 5 hydroxy, or optionally two geminal R⁴ are together oxo;

R⁶ is selected from the group consisting of hydrogen, halo, hydroxy, C₁₋₆alkoxy, C₁₋₆alkyl, C₂₋₆alkenyl, C₂₋₆alkynyl, alkyoxyalkyl, C₃₋₇cycloalkyl, aryl, arylalkyl, heteroaryl, heteroarylalkyl, heterocyclyl, heterocyclylalkyl, (R^{e}R^{f}N)alkyl, and R^{a}R^{b}N—, said C₁₋₆alkyl optionally substituted with one or more halo, —OR^{2b}, —C(═O)OR^{2b}, —C(═O)NHR^{2b}, —NHC(═NH)NHR^{2b}, —NHR^{2b}, SR^{2b}, imidazolyl, indolyl, —SCH₃, phenyl, and 4-hydroxyphenyl, and said C₁₋₆alkoxy, alkoxyalkyl, aryl, C₂₋₆alkenyl, C₂₋₆alkynyl, C₃₋₇cycloalkyl, arylalkyl, heterocyclyl, heteroaryl, heteroarylalkyl, heterocyclylalkyl, and alkyl in (R^{e}R^{f}N)alkyl are each optionally substituted with one or more R⁴;

R⁹ is selected from the group consisting of hydrogen and —C(═O)R^{9a};

R^{9a} is selected from the group consisting of —NR^{9b}R^{9c}, —OR^{9d}, C₁₋₆alkyl optionally substituted with up to 9 halo, and optionally substituted aryl;

R^{9b} is selected from the group consisting of hydrogen, C₁₋₆alkyl optionally substituted with up to 9 halo, and optionally substituted aryl;

R^{9c} is selected from the group consisting of C₁₋₆alkyl optionally substituted with up to 9 halo, and optionally substituted aryl; and

R^{9d} is selected from the group consisting of C₁₋₆alkyl optionally substituted with up to 9 halo, and optionally substituted aryl.

In some embodiments of Formula XV, L⁴ is selected from the group consisting of.

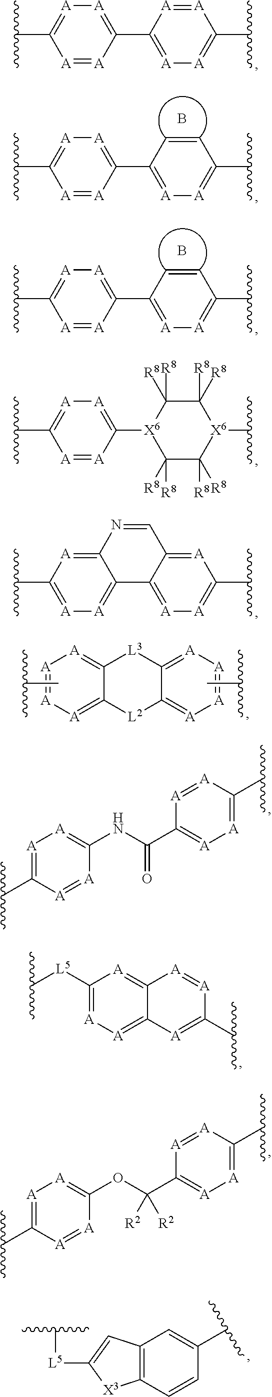

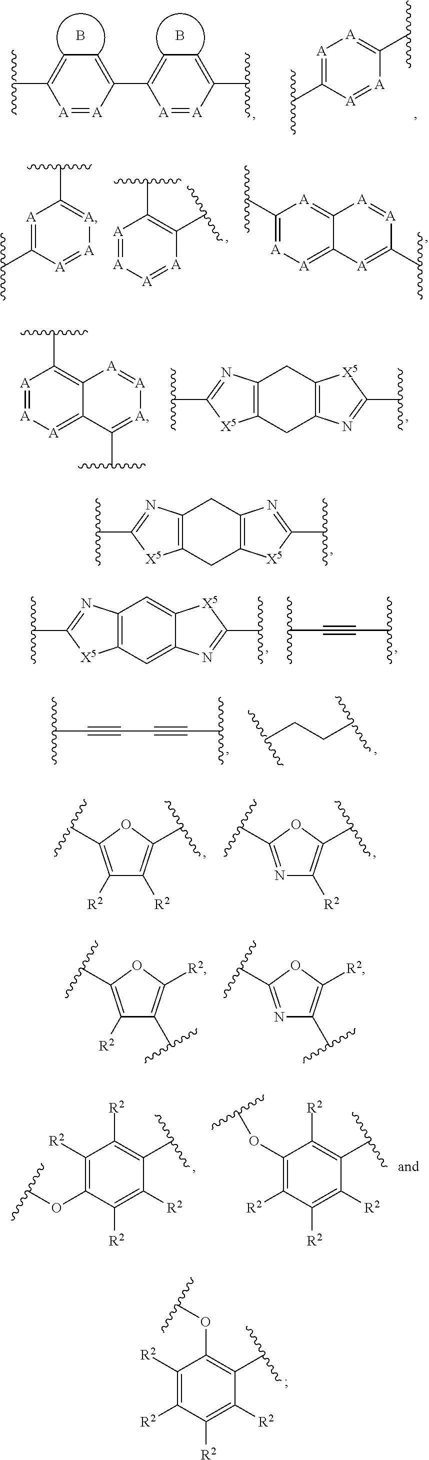

L² is selected from the group consisting of —C(═O)—, —(CH₂CH₂)—, —(CH₂O)—, —(CH₂S)—, —(CH═CH)—, —(CH═N)—, —NH—, O (oxygen), S (sulfur), and —CH₂—;

L³ is selected from the group consisting of

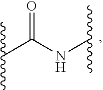

—(NR⁹)—, O (oxygen), S (sulfur), and —CH₂—;

L⁵ is selected from the group consisting of

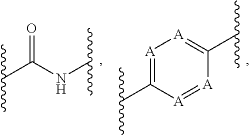

O (oxygen), NH, and —(CH═CH)—;

each X³ is separately selected from the group consisting of NH, O (oxygen), and S (sulfur);

each X⁵ is separately selected from the group consisting of —NH—, O (oxygen), S (sulfur), and —CH₂—;

each X⁶ is separately selected from the group consisting of N (nitrogen), and CR⁸; and

each B is separately selected, wherein B is a fused optionally substituted saturated or unsaturated three- to seven-membered carbocyclic ring or a fused optionally substituted saturated or unsaturated three- to seven-membered heterocyclic ring, each optionally substituted with one or more R⁴. In some embodiments, R⁶ is C₁₋₆alkyl optionally substituted with up to 9 halo.

In some embodiments of Formula XV, L⁶ is selected from the group consisting of.

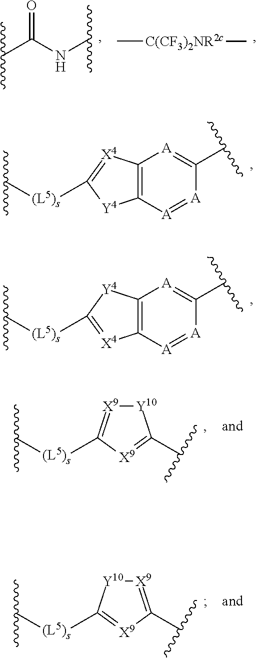

L⁷ is selected from the group consisting of

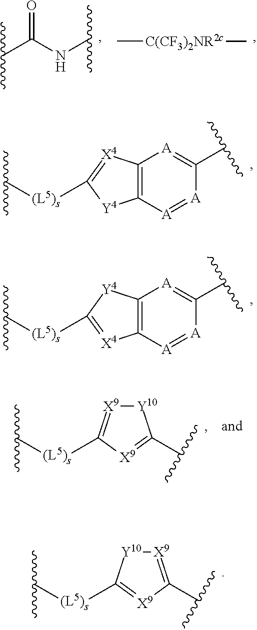

In some embodiments of Formula XV the compound is not selected from the group consisting of:

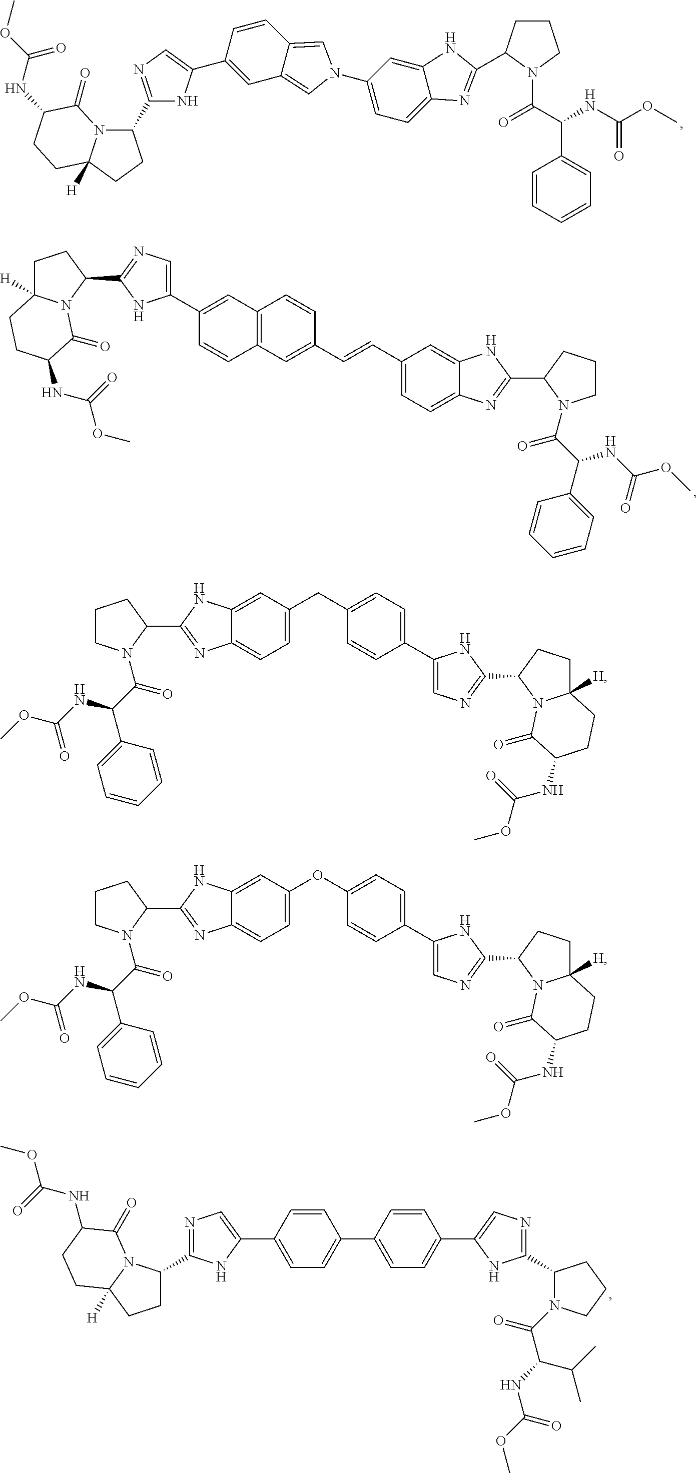

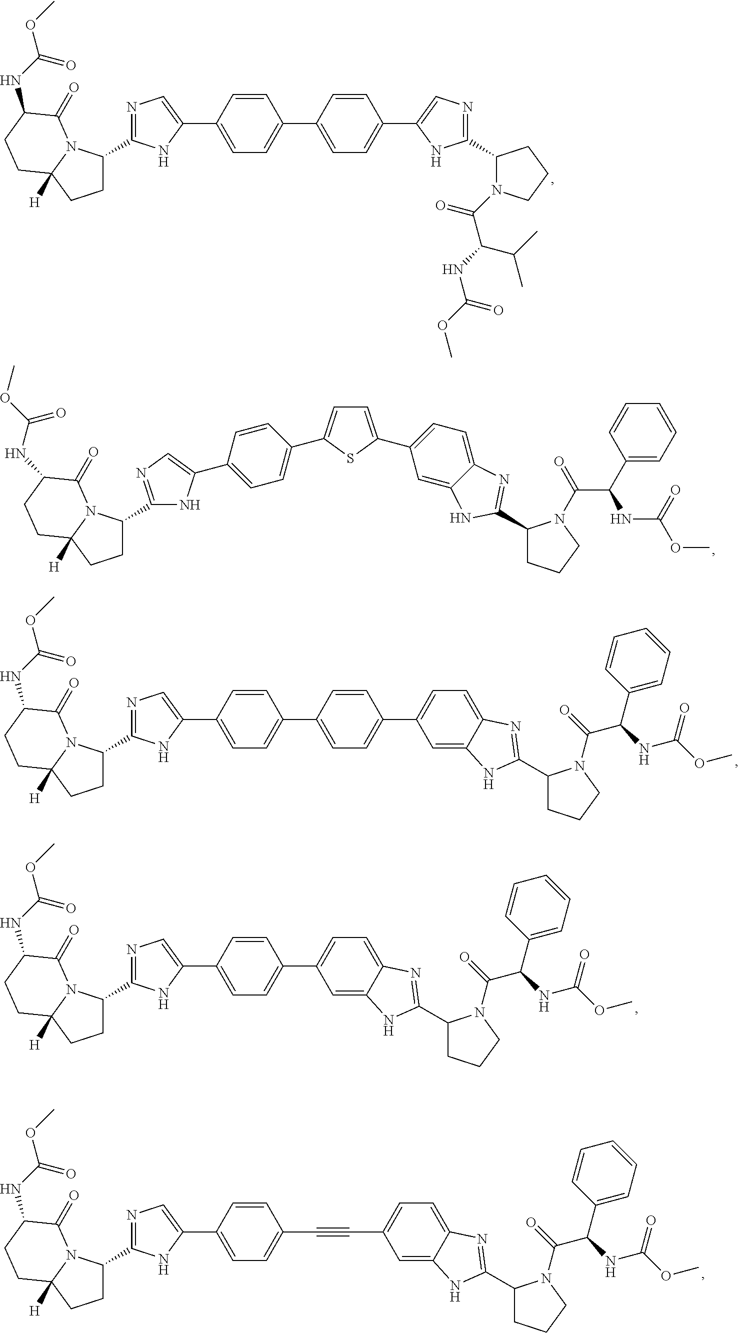

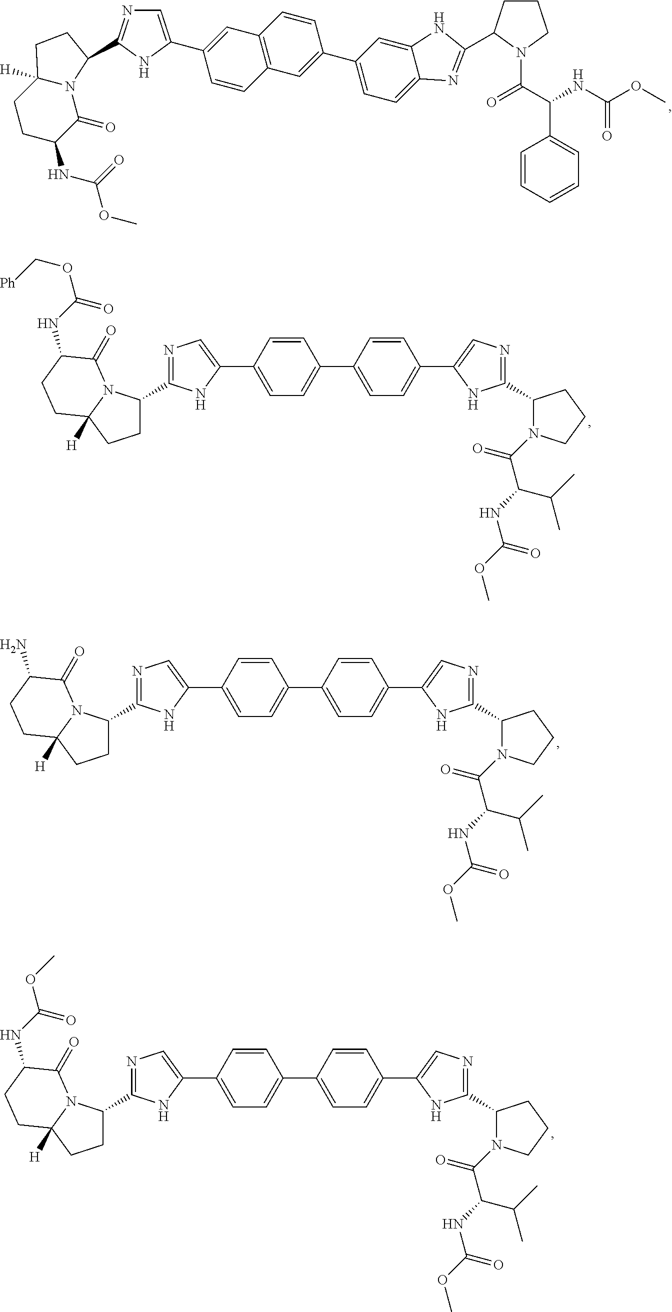

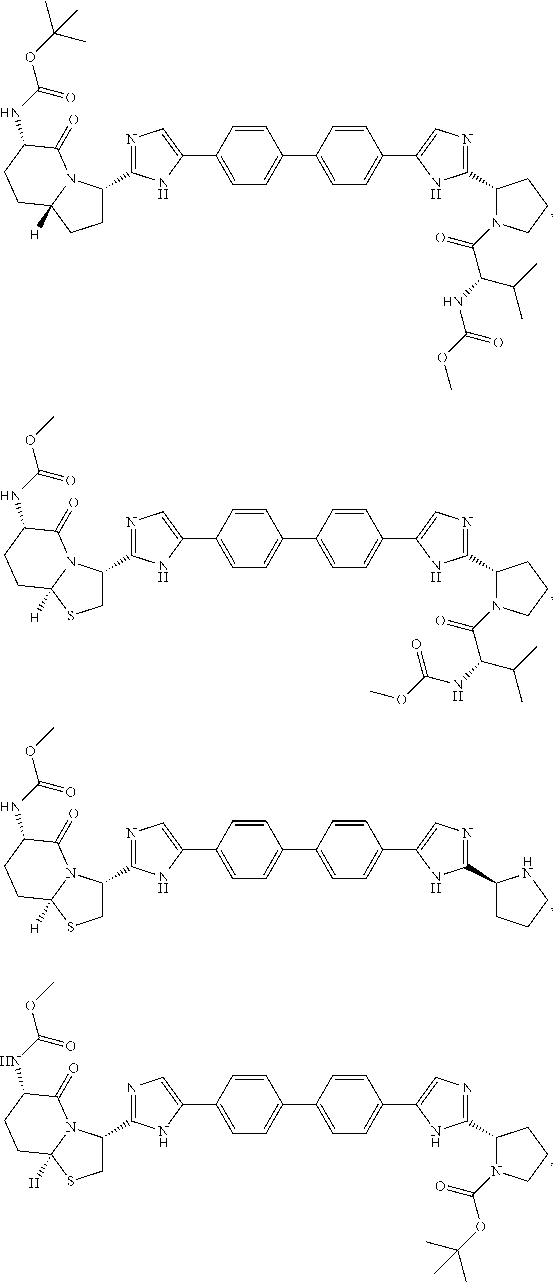

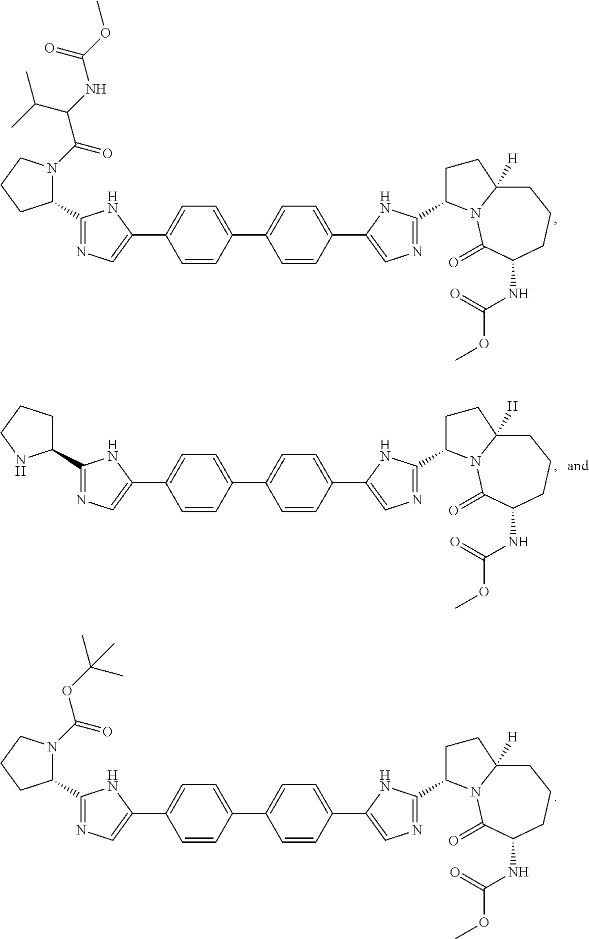

In some embodiments, the compound Formula XV has the structure

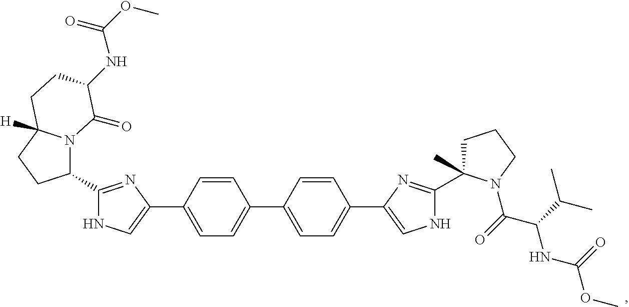

or a pharmaceutically acceptable salt thereof.

Some embodiments provide a pharmaceutical composition comprising a pharmaceutically acceptable excipient and a compound of Formulas VI, VII, VIII, IX, X, XI, XII, XIII, XIV, or XV.

Some embodiments provide a method of treating HCV infection in an individual, the method comprising administering to the individual an effective amount of a compound of Formulas VI, VII, VIII, IX, X, XI, XII, XIII, XIV, or XV or a pharmaceutical composition comprising a pharmaceutically acceptable excipient and a compound of Formulas VI, VII, VIII, IX, X, XI, XII, XIII, XIV, or XV.

Some embodiments provide a method of treating HCV infection in an individual, the method comprising administering to the individual an effective amount of a compound of Formulas VI, VII, VIII, IX, X, XI, XII, XIII, XIV, or XV or a pharmaceutical composition comprising a pharmaceutically acceptable excipient and a compound of Formulas VI, VII, VIII, IX, X, XI, XII, XIII, XIV, or XV. In some embodiments, the method further comprises identifying a subject suffering from a hepatitis C infection.

Some embodiments provide a method of treating liver fibrosis in an individual, the method comprising administering to the individual an effective amount of a compound of Formulas VI, VII, VIII, IX, X, XI, XII, XIII, XIV, or XV or a pharmaceutical composition comprising a pharmaceutically acceptable excipient and a compound of Formulas VI, VII, VIII, IX, X, XI, XII, XIII, XIV, or XV. In some embodiments, the method further comprises identifying a subject suffering from a hepatitis C infection.

Some embodiments provide a method of increasing liver function in an individual having a hepatitis C virus infection, the method comprising administering to the individual an effective amount of a compound of Formulas VI, VII, VIII, IX, X, XI, XII, XIII, XIV, or XV or a pharmaceutical composition comprising a pharmaceutically acceptable excipient and a compound of Formulas VI, VII, VIII, IX, X, XI, XII, XIII, XIV, or XV. In some embodiments, the method further comprises identifying a subject suffering from a hepatitis C infection.

### DETAILED DESCRIPTION OF THE PREFERRED EMBODIMENT

### Definitions

As used herein, common organic abbreviations are defined as follows:

| | |
|---|---|
| Ac | Acetyl |
| Ac₂O | Acetic anhydride |
| aq. | Aqueous |
| Bn | Benzyl |
| Bz | Benzoyl |
| BOC or Boc | tert-Butoxycarbonyl |
| Bu | n-Butyl |
| cat. | Catalytic |
| Cbz | Carbobenzyloxy |
| CDI | 1,1′-carbonyldiimidazole |
| Cy (c-C₆H₁₁) | Cyclohexyl |
| ° C. | Temperature in degrees Centigrade |
| DBU | 1,8-Diazabicyclo[5.4.0]undec-7-ene |
| DCE | 1,2-Dichloroethane |
| DCM | methylene chloride |
| DIEA | Diisopropylethylamine |
| DMA | Dimethylacetamide |
| DME | Dimethoxyethane |
| DMF | N,N′-Dimethylformamide |
| DMSO | Dimethylsulfoxide |
| Et | Ethyl |
| EtOAc | Ethyl acetate |
| g | Gram(s) |
| h | Hour (hours) |
| HATU | 2-(1H-7-azabenzotriazol-1-yl)-1,1,3,3-tetramethyl |
| | uronium hexafluorophosphate |
| HOBT | N-Hydroxybenzotriazole |
| iPr | Isopropyl |
| LCMS | Liquid chromatography-mass spectrometry |
| LDA | Lithium diisopropylamide |
| mCPBA | meta-Chloroperoxybenzoic Acid |
| MeOH | Methanol |
| MeCN | Acetonitrile |
| mL | Milliliter(s) |
| MTBE | Methyl tertiary-butyl ether |
| NH₄OAc | Ammonium acetate |
| PG | Protecting group |
| Pd/C | Palladium on activated carbon |
| Ph | Phenyl |
| ppt | Precipitate |
| RCM | Ring closing metathesis |
| rt | Room temperature |
| sBuLi | sec-Butylithium |
| TEA | Triethylamine |
| TCDI | 1,1′-Thiocarbonyl diimidazole |
| Tert, t | tertiary |
| TFA | Trifluoracetic acid |
| THF | Tetrahydrofuran |
| TLC | Thin-layer chromatography |
| TMEDA | Tetramethylethylenediamine |
| μL | Microliter(s) |

The terms “individual,” “host,” “subject,” and “patient” are used interchangeably herein, and refer to a mammal, including, but not limited to, primates, including simians and humans.

As used herein, the term “liver function” refers to a normal function of the liver, including, but not limited to, a synthetic function, including, but not limited to, synthesis of proteins such as serum proteins (e.g., albumin, clotting factors, alkaline phosphatase, aminotransferases (e.g., alanine transaminase, aspartate transaminase), 5′-nucleosidase, γ-glutaminyltranspeptidase, etc.), synthesis of bilirubin, synthesis of cholesterol, and synthesis of bile acids; a liver metabolic function, including, but not limited to, carbohydrate metabolism, amino acid and ammonia metabolism, hormone metabolism, and lipid metabolism; detoxification of exogenous drugs; a hemodynamic function, including splanchnic and portal hemodynamics; and the like.

The term “sustained viral response” (SVR; also referred to as a “sustained response” or a “durable response”), as used herein, refers to the response of an individual to a treatment regimen for HCV infection, in terms of serum HCV titer. Generally, a “sustained viral response” refers to no detectable HCV RNA (e.g., less than about 500, less than about 200, or less than about 100 genome copies per milliliter serum) found in the patient's serum for a period of at least about one month, at least about two months, at least about three months, at least about four months, at least about five months, or at least about six months following cessation of treatment.

“Treatment failure patients” as used herein generally refers to HCV-infected patients who failed to respond to previous therapy for HCV (referred to as “non-responders”) or who initially responded to previous therapy, but in whom the therapeutic response was not maintained (referred to as “relapsers”). The previous therapy generally can include treatment with IFN-α monotherapy or IFN-α combination therapy, where the combination therapy may include administration of IFN-α and an antiviral agent such as ribavirin.

As used herein, the terms “treatment,” “treating,” and the like, refer to obtaining a desired pharmacologic and/or physiologic effect. The effect may be prophylactic in terms of completely or partially preventing a disease or symptom thereof and/or may be therapeutic in terms of a partial or complete cure for a disease and/or adverse affect attributable to the disease. “Treatment,” as used herein, covers any treatment of a disease in a mammal, particularly in a human, and includes: (a) preventing the disease from occurring in a subject which may be predisposed to the disease but has not yet been diagnosed as having it; (b) inhibiting the disease, i.e., arresting its development; and (c) relieving the disease, i.e., causing regression of the disease.

As used herein, the term “alkyl” refers to a branched or unbranched fully saturated acyclic aliphatic hydrocarbon group (i.e. composed of carbon and hydrogen containing no double or triple bonds). In some embodiments, alkyls may be substituted or unsubstituted. Alkyls include, but are not limited to, methyl, ethyl, propyl, isopropyl, butyl, isobutyl, tertiary butyl, pentyl, hexyl, and the like, each of which may be optionally substituted in some embodiments.

As used herein, the term “heteroalkyl” refers to a branched or unbranched fully saturated acyclic aliphatic hydrocarbon group containing one or more heteroatoms in the carbon back bone (i.e., an alkyl group in which one or more carbon atoms is replaced with a heteroatom). In some embodiments, heteroalkyls may be substituted or unsubstituted. Heteroalkyls include, but are not limited to, ethers, thioethers, and alkyl-amino-alkyls.

The term “halo” used herein refers to fluoro, chloro, bromo, or iodo.

The term “alkoxy” used herein refers to straight or branched chain alkyl radical covalently bonded to the parent molecule through an —O— linkage. In some embodiments, alkoxys may be substituted or unsubstituted. Examples of alkoxy groups include, but are not limited to, methoxy, ethoxy, propoxy, isopropoxy, butoxy, n-butoxy, sec-butoxy, t-butoxy and the like.

The term “alkenyl” used herein refers to a monovalent straight or branched chain radical of from two to twenty carbon atoms containing at least one carbon-carbon double bond including, but not limited to, 1-propenyl, 2-propenyl, 2-methyl-1-propenyl, 1-butenyl, 2-butenyl, and the like. In some embodiments, alkenyls may be substituted or unsubstituted.

The term “alkynyl” used herein refers to a monovalent straight or branched chain radical of from two to twenty carbon atoms containing at least one carbon-carbon triple bond including, but not limited to, 1-propynyl, 1-butynyl, 2-butynyl, and the like. In some embodiments, alkynyls may be substituted or unsubstituted

The term “aryl” used herein refers to a homocyclic aromatic radical having one ring, two appended rings, or multiple fused rings. Examples of aryl groups include, but are not limited to, phenyl, naphthyl, biphenyl, phenanthrenyl, naphthacenyl, and the like. In some embodiments, aryls may be substituted or unsubstituted.

The term “cycloalkyl” used herein refers to a saturated aliphatic ring system radical having three to twenty carbon atoms including, but not limited to, cyclopropyl, cyclopentyl, cyclohexyl, cycloheptyl, and the like. In some embodiments, cycloalkyls may be substituted or unsubstituted.

The term “cycloalkenyl” used herein refers to an aliphatic ring system radical having three to twenty carbon atoms having at least one carbon-carbon double bond in the ring. Examples of cycloalkenyl groups include, but are not limited to, cyclopropenyl, cyclopentenyl, cyclohexenyl, cycloheptenyl, and the like. In some embodiments, cycloalkenyls may be substituted or unsubstituted.

The term “polycycloalkyl” used herein refers to saturated aliphatic ring system radical having at least two rings that are fused with or without bridgehead carbons. Examples of polycycloalkyl groups include, but are not limited to, bicyclo[4.4.0]decanyl, bicyclo[2.2.1]heptanyl, adamantyl, norbornyl, and the like.

The term “polycycloalkenyl” used herein refers to aliphatic ring system radical having at least two rings that are fused with or without bridgehead carbons in which at least one of the rings has a carbon-carbon double bond. Examples of polycycloalkenyl groups include, but are not limited to, norbornylenyl, 1,1′-bicyclopentenyl, and the like.

The term “polycyclic hydrocarbon” used herein refers to a ring system radical in which all of the ring members are carbon atoms. Polycyclic hydrocarbons can be aromatic or can contain less than the maximum number of non-cumulative double bonds. Examples of polycyclic hydrocarbon include, but are not limited to, naphthyl, dihydronaphthyl, indenyl, fluorenyl, and the like.

The term “heterocyclic” or “heterocyclyl” or “heterocycloalkyl” used herein refers to a cyclic ring system radical having at least one non-aromatic ring in which one or more ring atoms are not carbon, namely heteroatom. Monocyclic “heterocyclic” or “heterocyclyl” moieties are non-aromatic. Bicyclic “heterocyclic” or “heterocyclyl” moieties include one non-aromatic ring wherein at least one heteroatom is present in a ring. Tricyclic “heterocyclic” or “heterocyclyl” moieties include at least one non-aromatic ring wherein at least one heteroatom is present in a ring. Examples of heterocyclic groups include, but are not limited to, morpholinyl, tetrahydrofuranyl, dioxolanyl, pyrrolidinyl, oxazolyl, pyranyl, pyrrolyl, isoindoline and the like.

The term “heteroaryl” used herein refers to an aromatic ring system radical in which one or more ring atoms are not carbon, namely heteroatom, having one ring or multiple fused rings. In fused ring systems, the one or more heteroatoms may be present in only one of the rings. Examples of heteroaryl groups include, but are not limited to, benzothiazyl, benzoxazyl, quinazolinyl, quinolinyl, isoquinolinyl, quinoxalinyl, pyridinyl, pyrrolyl, oxazolyl, indolyl, and the like. In some embodiments, heteroaryls may be substituted or unsubstituted.

The term “heteroatom” used herein refers to, for example, oxygen, sulfur and nitrogen.

The term “arylalkyl” used herein refers to one or more aryl groups appended to an alkyl radical. Examples of arylalkyl groups include, but are not limited to, benzyl, phenethyl, phenpropyl, phenbutyl, and the like. In some embodiments, arylalkyls may be substituted or unsubstituted, and can be substituted on either the aryl or alkyl portion or on both.

The term “cycloalkylalkyl” used herein refers to one or more cycloalkyl groups appended to an alkyl radical. Examples of cycloalkylalkyl include, but are not limited to, cyclohexylmethyl, cyclohexylethyl, cyclopentylmethyl, cyclopentylethyl, and the like. In some embodiments, cycloalkylalkyls may be substituted or unsubstituted.

The term “heteroarylalkyl” used herein refers to one or more heteroaryl groups appended to an alkyl radical. Examples of heteroarylalkyl include, but are not limited to, pyridylmethyl, furanylmethyl, thiopheneylethyl, and the like. In some embodiments, heteroarylalkyls may be substituted or unsubstituted, and can be substituted on either the heteroaryl or alkyl portion or on both.

The term “heterocyclylalkyl” used herein refers to one or more heterocyclyl groups appended to an alkyl radical. Examples of heterocyclylalkyl include, but are not limited to, morpholinylmethyl, morpholinylethyl, morpholinylpropyl, tetrahydrofuranylmethyl, pyrrolidinylpropyl, and the like.

he term “aryloxy” used herein refers to an aryl radical covalently bonded to the parent molecule through an —O— linkage.

The term “alkylthio” used herein refers to straight or branched chain alkyl radical covalently bonded to the parent molecule through an —S— linkage. Examples of alkylthio groups include, but are not limited to, methanesulfide, ethanesulfide, propanesulfide, isopropanesulfide, butanesulfide, n-butanesulfide, sec-butanesulfide, tert-butanesulfide and the like.

The term “arylthio” used herein refers to an aryl radical covalently bonded to the parent molecule through an —S— linkage.

The term “alkylamino” used herein refers to nitrogen radical with one or more alkyl groups attached thereto. Thus, monoalkylamino refers to nitrogen radical with one alkyl group attached thereto and dialkylamino refers to nitrogen radical with two alkyl groups attached thereto.

The term “cyanoamino” used herein refers to nitrogen radical with nitrile group attached thereto.

The term “carbamyl” used herein refers to RNHCOO—.

The term “keto” and “carbonyl” used herein refers to C═O.

The term “carboxy” used herein refers to —COOH.

The term “sulfamyl” used herein refers to —SO₂NH₂.

The term “sulfonyl” used herein refers to —SO₂—.

The term “sulfinyl” used herein refers to —SO—.

The term “thiocarbonyl” used herein refers to C═S.

The term “thiocarboxy” used herein refers to CSOH.

The term “sulfonamide” used herein refers to —SO₂NR′₂ where each R′ is individually selected from H (hydrogen), C₁-C₆ alkyl, C₃-C₇ cycloalkyl, arylalkyl and aryl optionally substituted with C₁-C₆ alkyl.

The term “ester” used herein refers to —COOR′ where R′ is selected from C₁-C₆ alkyl, C₃-C₇ cycloalkyl, arylalkyl and aryl optionally substituted with C₁-C₆ alkyl.

The term “C-amide” used herein refers to —C(═O)NR′₂ where each R′ is individually selected from H (hydrogen), C₁-C₆ alkyl, C₃-C₇ cycloalkyl, arylalkyl and aryl optionally substituted with C₁-C₆ alkyl.

The term “N-amide” used herein refers to —NR′C(═O)R′ where each R′ is individually selected from H (hydrogen), C₁-C₆ alkyl, C₃-C₇ cycloalkyl, arylalkyl and aryl optionally substituted with C₁-C₆ alkyl.

The term “N-carbamate” used herein refers to —NR′C(═O)OR′ where each R′ is individually selected from H (hydrogen), C₁-C₆ alkyl, C₃-C₇ cycloalkyl, arylalkyl and aryl optionally substituted with C₁-C₆ alkyl.

The term “O-carbamate” used herein refers to —OC(═O)NR′₂ where each R′ is individually selected from H (hydrogen), C₁-C₆ alkyl, C₃-C₇ cycloalkyl, arylalkyl and aryl optionally substituted with C₁-C₆ alkyl.

The term “urea” used herein refers to —NR′C(═O)NR′₂ where each R′ is individually selected from H (hydrogen), C₁-C₆ alkyl, C₃-C₇ cycloalkyl, arylalkyl and aryl optionally substituted with C₁-C₆ alkyl.

As used herein, a radical indicates a species with one or more, unpaired electron such that the species containing the radical can be covalently bonded to one or more other species. Hence, in this context, a radical is not necessarily a free radical. Rather, a radical indicates a specific portion of a larger molecule. The term “radical” can be used interchangeably with the term “moiety” or “group.”

As used herein, a substituted group is derived from the unsubstituted parent structure in which there has been an exchange of one or more hydrogen atoms for another atom or group. When substituted, the substituent group(s) is (are) one or more group(s) individually and independently selected from C₁-C₆ alkyl, C₁-C₆ alkenyl, C₁-C₆ alkynyl, C₃-C₇ cycloalkyl (optionally substituted with halo, alkyl, alkoxy, carboxyl, haloalkyl, CN, —SO₂-alkyl, —CF₃, and —OCF₃), cycloalkyl geminally attached, C₁-C₆ heteroalkyl, C₃-C₁₀ heterocycloalkyl (e.g., tetrahydrofuryl) (optionally substituted with halo, alkyl, alkoxy, carboxyl, CN, —SO₂-alkyl, —CF₃, and —OCF₃), aryl (optionally substituted with halo, alkyl, aryl optionally substituted with C₁-C₆ alkyl, arylalkyl, alkoxy, carboxyl, CN, —SO₂-alkyl, —CF₃, and —OCF₃), arylalkyl (optionally substituted with halo, alkyl, alkoxy, aryl, carboxyl, CN, —SO₂-alkyl, —CF₃, and —OCF₃), heteroaryl (optionally substituted with halo, alkyl, alkoxy, aryl, aralkyl, carboxyl, CN, —SO₂-alkyl, —CF₃, and —OCF₃), halo (e.g., chloro, bromo, iodo and fluoro), cyano, hydroxy, —CF₃, C₁-C₆ alkoxy, aryloxy, sulfhydryl (mercapto), halo(C₁-C₆)alkyl, C₁-C₆ alkylthio, arylthio, mono- and di-(C₁-C₆)alkyl amino, quaternary ammonium salts, amino(C₁-C₆)alkoxy, hydroxy(C₁-C₆)alkylamino, amino(C₁-C₆)alkylthio, cyanoamino, nitro, carbamyl, keto (oxy), carbonyl, carboxy, glycolyl, glycyl, hydrazino, guanyl, sulfamyl, sulfonyl, sulfinyl, thiocarbonyl, thiocarboxy, sulfonamide, ester, C-amide, N-amide, N-carbamate, O-carbamate, and urea. The protecting groups that can form the protective derivatives of the above substituents are known to those of skill in the art and can be found in references such as Greene and Wuts Protective Groups in Organic Synthesis; John Wiley and Sons: New York, 1999. Wherever a substituent is described as “optionally substituted” that substituent can be substituted with the above substituents.

Asymmetric carbon atoms may be present in the compounds described. All such isomers, including diastereomers and enantiomers, as well as the mixtures thereof are intended to be included in the scope of the recited compound. In certain cases, compounds can exist in tautomeric forms. All tautomeric forms are intended to be included in the scope Likewise, when compounds contain an alkenyl or alkenylene group, there exists the possibility of cis- and trans-isomeric forms of the compounds. Both cis- and trans-isomers, as well as the mixtures of cis- and trans-isomers, are contemplated. Thus, reference herein to a compound includes all of the aforementioned isomeric forms unless the context clearly dictates otherwise.

Various forms are included in the embodiments, including polymorphs, solvates, hydrates, conformers, salts, and prodrug derivatives. A polymorph is a composition having the same chemical formula, but a different structure. A solvate is a composition formed by solvation (the combination of solvent molecules with molecules or ions of the solute). A hydrate is a compound formed by an incorporation of water. A conformer is a structure that is a conformational isomer. Conformational isomerism is the phenomenon of molecules with the same structural formula but different conformations (conformers) of atoms about a rotating bond. Salts of compounds can be prepared by methods known to those skilled in the art. For example, salts of compounds can be prepared by reacting the appropriate base or acid with a stoichiometric equivalent of the compound. A prodrug is a compound that undergoes biotransformation (chemical conversion) before exhibiting its pharmacological effects. For example, a prodrug can thus be viewed as a drug containing specialized protective groups used in a transient manner to alter or to eliminate undesirable properties in the parent molecule. Thus, reference herein to a compound includes all of the aforementioned forms unless the context clearly dictates otherwise.

The term “pharmaceutically acceptable salt,” as used herein, and particularly when referring to a pharmaceutically acceptable salt of a compound, including a compound of Formulas VI, VII, VIII, IX, X, XI, XII, XIII, XIV, or XV, as produced and synthesized by the methods disclosed herein, refers to any pharmaceutically acceptable salts of a compound, and preferably refers to an acid addition salt of a compound. With respect to compounds synthesized by the method of this embodiment that contain a basic nitrogen, the preferred examples of pharmaceutically acceptable salts are acid addition salts of pharmaceutically acceptable inorganic or organic acids, including, but not limited to, hydrohalic, sulfuric, phosphoric, aliphatic or aromatic carboxylic, or sulfonic acid. Examples of pharmaceutically acceptable inorganic or organic acids as a component of an addition salt, include but are not limited to, hydrochloric acid, hydrobromic acid, phosphoric acid, sulfuric acid acetic acid, succinic acid, lactic acid, malic acid, tartaric acid, citric acid, ascorbi acid c, nicotinic acid, methanesulfonic acid, p-toluensulfonic acid or naphthalenesulfonic acid acid. With respect to compounds synthesized by the methods of this embodiment that contain an acidic functional group, the preferred examples of pharmaceutically acceptable salts include, but are not limited to, alkali metal salts (sodium or potassium), alkaline earth metal salts (calcium or magnesium), or ammonium salts derived from ammonia or from pharmaceutically acceptable organic amines, for example C₁-C₇ alkylamine, cyclohexylamine, triethanolamine, ethylenediamine or tris-(hydroxymethyl)-aminomethane.

Isotopes may be present in the compounds described. Each chemical element as represented in a compound structure may include any isotope of said element. For example, in a compound structure a hydrogen atom may be explicitely disclosed or understood to be present in the compound. At any position of the compound that a hydrogen atom may be present, the hydrogen atom can be any isotope of hydrogen, including but not limited to hydrogen-1 (protium) and hydrogen-2 (deuterium). Thus, reference herein to a compound encompasses all potential isotopic forms unless the context clearly dictates otherwise.

Wherever a substituent as depicted as a di-radical (i.e., has two points of attachment to the rest of the molecule), it is to be understood that the substituent can be attached in any directional configuration unless otherwise indicated. Thus, for example, a substituent depicted as -AE- or

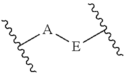

includes the substituent being oriented such that the A is attached at the leftmost attachment point of the molecule as well as the case in which A is attached at the rightmost attachment point of the molecule.

It is to be understood that certain radical naming conventions can include either a mono-radical or a di-radical, depending on the context. For example, where a substituent requires two points of attachment to the rest of the molecule, it is understood that the substituent is a di-radical. A substituent identified as alkyl, that requires two points of attachment, includes di-radicals such as —CH₂—, —CH₂CH₂—, —CH₂CH(CH₃)CH₂—, and the like; a substituent depicted as alkoxy that requires two points of attachment, includes di-radicals such as —OCH₂—, —OCH₂CH₂—, —OCH₂CH(CH₃)CH₂—, and the like: and a substituent depicted as arylC(═O)— that requires two points of attachment, includes di-radicals such as

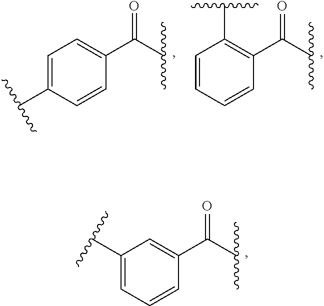

and the like.

Where a range of values is provided, it is understood that the upper and lower limit, and each intervening value between the upper and lower limit of the range is encompassed within the embodiments.

Unless defined otherwise, all technical and scientific terms used herein have the same meaning as commonly understood by one of ordinary skill in the art to which the embodiments belong. Although any methods and materials similar or equivalent to those described herein can also be used in the practice or testing of the embodiments, the preferred methods and materials are now described. All publications mentioned herein are incorporated herein by reference to disclose and describe the methods and/or materials in connection with which the publications are cited.

It must be noted that as used herein and in the appended claims, the singular forms “a,” “and,” and “the” include plural referents unless the context clearly dictates otherwise. Thus, for example, reference to “a method” includes a plurality of such methods and reference to “a dose” includes reference to one or more doses and equivalents thereof known to those skilled in the art, and so forth.

### Compounds

The present embodiments provide compounds of Formulas VI, VII, VIII, IX, X, XI, XII, XIII, XIV, or XV, as defined above, as well as pharmaceutical compositions and formulations comprising any compound of Formulas VI, VII, VIII, IX, X, XI, XII, XIII, XIV, or XV. A subject compound is useful for treating HCV infection and other disorders, as discussed below.

In many embodiments, a subject compound inhibits HCV viral replication. For example, a subject compound inhibits HCV viral replication by at least about 10%, at least about 15%, at least about 20%, at least about 25%, at least about 30%, at least about 40%, at least about 50%, at least about 60%, at least about 70%, at least about 80%, or at least about 90%, or more, compared to HCV viral replication in the absence of the compound. Whether a subject compound inhibits HCV viral replication can be determined using methods known in the art, including an in vitro viral replication assay.

### Compositions

The present embodiments further provide compositions, including pharmaceutical compositions, comprising compounds of the general Formulas VI, VII, VIII, IX, X, XI, XII, XIII, XIV, or XV.

A subject pharmaceutical composition comprises a subject compound; and a pharmaceutically acceptable excipient. A wide variety of pharmaceutically acceptable excipients is known in the art and need not be discussed in detail herein. Pharmaceutically acceptable excipients have been amply described in a variety of publications, including, for example, A. Gennaro (2000) “Remington: The Science and Practice of Pharmacy,” 20th edition, Lippincott, Williams, & Wilkins; Pharmaceutical Dosage Forms and Drug Delivery Systems (1999) H. C. Ansel et al., eds., 7^{th} ed., Lippincott, Williams, & Wilkins; and Handbook of Pharmaceutical Excipients (2000) A. H. Kibbe et al., eds., 3^{rd} ed. Amer. Pharmaceutical Assoc.

The pharmaceutically acceptable excipients, such as vehicles, adjuvants, carriers or diluents, are known in the art. Moreover, pharmaceutically acceptable auxiliary substances, such as pH adjusting and buffering agents, tonicity adjusting agents, stabilizers, wetting agents and the like, are known in the art.

In some embodiments, a compound as described herein is formulated in an aqueous buffer. Suitable aqueous buffers include, but are not limited to, acetate, succinate, citrate, and phosphate buffers varying in strengths from about 5 mM to about 100 mM. In some embodiments, the aqueous buffer includes reagents that provide for an isotonic solution. Such reagents include, but are not limited to, sodium chloride; and sugars e.g., mannitol, dextrose, sucrose, and the like. In some embodiments, the aqueous buffer further includes a non-ionic surfactant such as polysorbate 20 or 80. Optionally the formulations may further include a preservative. Suitable preservatives include, but are not limited to, a benzyl alcohol, phenol, chlorobutanol, benzalkonium chloride, and the like. In many cases, the formulation is stored at about 4° C. Formulations may also be lyophilized, in which case they generally include cryoprotectants such as sucrose, trehalose, lactose, maltose, mannitol, and the like. Lyophilized formulations can be stored over extended periods of time, even at ambient temperatures.

As such, administration of the compounds as described herein can be achieved in various ways, including oral, buccal, rectal, parenteral, intraperitoneal, intradermal, subcutaneous, intramuscular, transdermal, intratracheal, etc., administration. In some embodiments, administration is by bolus injection, e.g., subcutaneous bolus injection, intramuscular bolus injection, and the like.

The pharmaceutical compositions of the embodiments can be administered orally, parenterally or via an implanted reservoir. Oral administration or administration by injection is preferred.

Subcutaneous administration of a pharmaceutical composition of the embodiments is accomplished using standard methods and devices, e.g., needle and syringe, a subcutaneous injection port delivery system, and the like. See, e.g., U.S. Pat. Nos. 3,547,119; 4,755,173; 4,531,937; 4,311,137; and 6,017,328. A combination of a subcutaneous injection port and a device for administration of a pharmaceutical composition of the embodiments to a patient through the port is referred to herein as “a subcutaneous injection port delivery system.” In many embodiments, subcutaneous administration is achieved by bolus delivery by needle and syringe.

In pharmaceutical dosage forms, the compounds as described herein may be administered in the form of their pharmaceutically acceptable salts, or they may also be used alone or in appropriate association, as well as in combination, with other pharmaceutically active compounds. The following methods and excipients are merely exemplary and are in no way limiting.

For oral preparations, the compounds as described herein can be used alone or in combination with appropriate additives to make tablets, powders, granules or capsules, for example, with conventional additives, such as lactose, mannitol, corn starch or potato starch; with binders, such as crystalline cellulose, cellulose derivatives, acacia, corn starch or gelatins; with disintegrators, such as corn starch, potato starch or sodium carboxymethylcellulose; with lubricants, such as talc or magnesium stearate; and if desired, with diluents, buffering agents, moistening agents, preservatives and flavoring agents.

The compounds as described herein can be formulated into preparations for injection by dissolving, suspending or emulsifying them in an aqueous or nonaqueous solvent, such as vegetable or other similar oils, synthetic aliphatic acid glycerides, esters of higher aliphatic acids or propylene glycol; and if desired, with conventional additives such as solubilizers, isotonic agents, suspending agents, emulsifying agents, stabilizers and preservatives.

Furthermore, the compounds as described herein can be made into suppositories by mixing with a variety of bases such as emulsifying bases or water-soluble bases. The compounds of the embodiments can be administered rectally via a suppository. The suppository can include vehicles such as cocoa butter, carbowaxes and polyethylene glycols, which melt at body temperature, yet are solidified at room temperature.

Unit dosage forms for oral or rectal administration such as syrups, elixirs, and suspensions may be provided wherein each dosage unit, for example, teaspoonful, tablespoonful, tablet or suppository, contains a predetermined amount of the composition containing one or more compounds as described herein. Similarly, unit dosage forms for injection or intravenous administration may comprise the compounds as described herein in a composition as a solution in sterile water, normal saline or another pharmaceutically acceptable carrier.

The term “unit dosage form,” as used herein, refers to physically discrete units suitable as unitary dosages for human and animal subjects, each unit containing a predetermined quantity of compounds of the embodiments calculated in an amount sufficient to produce the desired effect in association with a pharmaceutically acceptable diluent, carrier or vehicle. The specifications for the novel unit dosage forms of the embodiments depend on the particular compound employed and the effect to be achieved, and the pharmacodynamics associated with each compound in the host.

The pharmaceutically acceptable excipients, such as vehicles, adjuvants, carriers or diluents, are readily known in the art. Moreover, pharmaceutically acceptable auxiliary substances, such as pH adjusting and buffering agents, tonicity adjusting agents, stabilizers, wetting agents and the like, are readily known in the art.

### Treating a Hepatitis Virus Infection

The methods and compositions described herein are generally useful in treatment of an of HCV infection.

Preferred embodiments provide a method of treating a hepatitis C virus infection in an individual, the method comprising administering to the individual an effective amount of a composition comprising a subject compound.

Preferred embodiments provide a method of treating liver fibrosis in an individual, the method comprising administering to the individual an effective amount of a composition comprising a subject compound.

Preferred embodiments provide a method of increasing liver function in an individual having a hepatitis C virus infection, the method comprising administering to the individual an effective amount of a composition comprising a subject compound.

Whether a subject method is effective in treating an HCV infection can be determined by a reduction in viral load, a reduction in time to seroconversion (virus undetectable in patient serum), an increase in the rate of sustained viral response to therapy, a reduction of morbidity or mortality in clinical outcomes, or other indicator of disease response.

In general, an effective amount of a compound of Formulas VI, VII, VIII, IX, X, XI, XII, XIII, XIV, or XV, and optionally one or more additional antiviral agents, is an amount that is effective to reduce viral load or achieve a sustained viral response to therapy.

Whether a subject method is effective in treating an HCV infection can be determined by measuring viral load, or by measuring a parameter associated with HCV infection, including, but not limited to, liver fibrosis, elevations in serum transaminase levels, and necroinflammatory activity in the liver. Indicators of liver fibrosis are discussed in detail below.

In some embodiments, the methods involve administering an effective amount of a compound of Formulas VI, VII, VIII, IX, X, XI, XII, XIII, XIV, or XV, optionally in combination with an effective amount of one or more additional antiviral agents. In some embodiments, an effective amount of a compound of Formulas VI, VII, VIII, IX, X, XI, XII, XIII, XIV, or XV, and optionally one or more additional antiviral agents, is an amount that is effective to reduce viral titers to undetectable levels, e.g., to about 1000 to about 5000, to about 500 to about 1000, or to about 100 to about 500 genome copies/mL serum. In some embodiments, an effective amount of a compound of Formulas VI, VII, VIII, IX, X, XI, XII, XIII, XIV, or XV, and optionally one or more additional antiviral agents, is an amount that is effective to reduce viral load to lower than 100 genome copies/mL serum.

In some embodiments, an effective amount of a compound of Formulas VI, VII, VIII, IX, X, XI, XII, XIII, XIV, or XV, and optionally one or more additional antiviral agents, is an amount that is effective to achieve a 1.5-log, a 2-log, a 2.5-log, a 3-log, a 3.5-log, a 4-log, a 4.5-log, or a 5-log reduction in viral titer in the serum of the individual.

In many embodiments, an effective amount of a compound of Formulas VI, VII, VIII, IX, X, XI, XII, XIII, XIV, or XV, and optionally one or more additional antiviral agents, is an amount that is effective to achieve a sustained viral response, e.g., non-detectable or substantially non-detectable HCV RNA (e.g., less than about 500, less than about 400, less than about 200, or less than about 100 genome copies per milliliter serum) is found in the patient's serum for a period of at least about one month, at least about two months, at least about three months, at least about four months, at least about five months, or at least about six months following cessation of therapy.

As noted above, whether a subject method is effective in treating an HCV infection can be determined by measuring a parameter associated with HCV infection, such as liver fibrosis. Methods of determining the extent of liver fibrosis are discussed in detail below. In some embodiments, the level of a serum marker of liver fibrosis indicates the degree of liver fibrosis.

As one non-limiting example, levels of serum alanine aminotransferase (ALT) are measured, using standard assays. In general, an ALT level of less than about 45 international units is considered normal. In some embodiments, an effective amount of a compound of Formulas VI, VII, VIII, IX, X, XI, XII, XIII, XIV, or XV, and optionally one or more additional antiviral agents, is an amount effective to reduce ALT levels to less than about 45 IU/mL serum.

A therapeutically effective amount of a compound of Formulas VI, VII, VIII, IX, X, XI, XII, XIII, XIV, or XV, and optionally one or more additional antiviral agents, is an amount that is effective to reduce a serum level of a marker of liver fibrosis by at least about 10%, at least about 20%, at least about 25%, at least about 30%, at least about 35%, at least about 40%, at least about 45%, at least about 50%, at least about 55%, at least about 60%, at least about 65%, at least about 70%, at least about 75%, or at least about 80%, or more, compared to the level of the marker in an untreated individual, or to a placebo-treated individual. Methods of measuring serum markers include immunological-based methods, e.g., enzyme-linked immunosorbent assays (ELISA), radioimmunoassays, and the like, using antibody specific for a given serum marker.

In many embodiments, an effective amount of a compound of Formulas VI, VII, VIII, IX, X, XI, XII, XIII, XIV, or XV and an additional antiviral agent is a synergistic amount. As used herein, a “synergistic combination” or a “synergistic amount” of a compound of Formulas VI, VII, VIII, IX, X, XI, XII, XIII, XIV, or XV and an additional antiviral agent is a combined dosage that is more effective in the therapeutic or prophylactic treatment of an HCV infection than the incremental improvement in treatment outcome that could be predicted or expected from a merely additive combination of (i) the therapeutic or prophylactic benefit of the compound of Formulas VI, VII, VIII, IX, X, XI, XII, XIII, XIV, or XV when administered at that same dosage as a monotherapy and (ii) the therapeutic or prophylactic benefit of the additional antiviral agent when administered at the same dosage as a monotherapy.

In some embodiments, a selected amount of a compound of Formulas VI, VII, VIII, IX, X, XI, XII, XIII, XIV, or XV and a selected amount of an additional antiviral agent are effective when used in combination therapy for a disease, but the selected amount of the compound of Formulas VI, VII, VIII, IX, X, XI, XII, XIII, XIV, or XV and/or the selected amount of the additional antiviral agent is less effective when used in monotherapy for the disease. Thus, the embodiments encompass (1) regimens in which a selected amount of the additional antiviral agent enhances the therapeutic benefit of a selected amount of the compound of Formulas VI, VII, VIII, IX, X, XI, XII, XIII, XIV, or XV when used in combination therapy for a disease, where the selected amount of the additional antiviral agent provides negligible therapeutic benefit when used in monotherapy for the disease (2) regimens in which a selected amount of the compound of Formulas VI, VII, VIII, IX, X, XI, XII, XIII, XIV, or XV enhances the therapeutic benefit of a selected amount of the additional antiviral agent when used in combination therapy for a disease, where the selected amount of the compound of Formulas VI, VII, VIII, IX, X, XI, XII, XIII, XIV, or XV provides negligible therapeutic benefit when used in monotherapy for the disease and (3) regimens in which a selected amount of the compound of Formulas VI, VII, VIII, IX, X, XI, XII, XIII, XIV, or XV and a selected amount of the additional antiviral agent provide a therapeutic benefit when used in combination therapy for a disease, where each of the selected amounts of the compound of Formulas VI, VII, VIII, IX, X, XI, XII, XIII, XIV, or XV and the additional antiviral agent, respectively, provides negligible therapeutic benefit when used in monotherapy for the disease. As used herein, a “synergistically effective amount” of a compound of Formulas VI, VII, VIII, IX, X, XI, XII, XIII, XIV, or XV and an additional antiviral agent, and its grammatical equivalents, shall be understood to include any regimen encompassed by any of (1)-(3) above.

### Fibrosis

The embodiments provides methods for treating liver fibrosis (including forms of liver fibrosis resulting from, or associated with, HCV infection), generally involving administering a therapeutic amount of a compound of Formulas VI, VII, VIII, IX, X, XI, XII, XIII, XIV, or XV, and optionally one or more additional antiviral agents. Effective amounts of compounds of Formulas VI, VII, VIII, IX, X, XI, XII, XIII, XIV, or XV, with and without one or more additional antiviral agents, as well as dosing regimens, are as discussed below.

Whether treatment with a compound of Formulas VI, VII, VIII, IX, X, XI, XII, XIII, XIV, or XV, and optionally one or more additional antiviral agents, is effective in reducing liver fibrosis can be determined by any of a number of well-established techniques for measuring liver fibrosis and liver function. Liver fibrosis reduction is determined by analyzing a liver biopsy sample. An analysis of a liver biopsy comprises assessments of two major components: necroinflammation assessed by “grade” as a measure of the severity and ongoing disease activity, and the lesions of fibrosis and parenchymal or vascular remodeling as assessed by “stage” as being reflective of long-term disease progression. See, e.g., Brunt (2000) Hepatol. 31:241-246; and METAVIR (1994) Hepatology 20:15-20. Based on analysis of the liver biopsy, a score is assigned. A number of standardized scoring systems exist which provide a quantitative assessment of the degree and severity of fibrosis. These include the METAVIR, Knodell, Scheuer, Ludwig, and Ishak scoring systems.

The METAVIR scoring system is based on an analysis of various features of a liver biopsy, including fibrosis (portal fibrosis, centrilobular fibrosis, and cirrhosis); necrosis (piecemeal and lobular necrosis, acidophilic retraction, and ballooning degeneration); inflammation (portal tract inflammation, portal lymphoid aggregates, and distribution of portal inflammation); bile duct changes; and the Knodell index (scores of periportal necrosis, lobular necrosis, portal inflammation, fibrosis, and overall disease activity). The definitions of each stage in the METAVIR system are as follows: score: 0, no fibrosis; score: 1, stellate enlargement of portal tract but without septa formation; score: 2, enlargement of portal tract with rare septa formation; score: 3, numerous septa without cirrhosis; and score: 4, cirrhosis.

Knodell's scoring system, also called the Hepatitis Activity Index, classifies specimens based on scores in four categories of histologic features: I. Periportal and/or bridging necrosis; II. Intralobular degeneration and focal necrosis; III. Portal inflammation; and IV. Fibrosis. In the Knodell staging system, scores are as follows: score: 0, no fibrosis; score: 1, mild fibrosis (fibrous portal expansion); score: 2, moderate fibrosis; score: 3, severe fibrosis (bridging fibrosis); and score: 4, cirrhosis. The higher the score, the more severe the liver tissue damage. Knodell (1981) Hepatol. 1:431.

The Scheuer scoring system scores are as follows: score: 0, no fibrosis; score: 1, enlarged, fibrotic portal tracts; score: 2, periportal or portal-portal septa, but intact architecture; score: 3, fibrosis with architectural distortion, but no obvious cirrhosis; score: 4, probable or definite cirrhosis. Scheuer (1991) J. Hepatol. 13:372.

The Ishak scoring system is described in Ishak (1995) J. Hepatol. 22:696-699. Stage 0, No fibrosis; Stage 1, Fibrous expansion of some portal areas, with or without short fibrous septa; stage 2, Fibrous expansion of most portal areas, with or without short fibrous septa; stage 3, Fibrous expansion of most portal areas with occasional portal to portal (P-P) bridging; stage 4, Fibrous expansion of portal areas with marked bridging (P-P) as well as portal-central (P-C); stage 5, Marked bridging (P-P and/or P-C) with occasional nodules (incomplete cirrhosis); stage 6, Cirrhosis, probable or definite.

The benefit of anti-fibrotic therapy can also be measured and assessed by using the Child-Pugh scoring system which comprises a multicomponent point system based upon abnormalities in serum bilirubin level, serum albumin level, prothrombin time, the presence and severity of ascites, and the presence and severity of encephalopathy. Based upon the presence and severity of abnormality of these parameters, patients may be placed in one of three categories of increasing severity of clinical disease: A, B, or C.

In some embodiments, a therapeutically effective amount of a compound of Formulas VI, VII, VIII, IX, X, XI, XII, XIII, XIV, or XV, and optionally one or more additional antiviral agents, is an amount that effects a change of one unit or more in the fibrosis stage based on pre- and post-therapy liver biopsies. In particular embodiments, a therapeutically effective amount of a compound of Formulas VI, VII, VIII, IX, X, XI, XII, XIII, XIV, or XV, and optionally one or more additional antiviral agents, reduces liver fibrosis by at least one unit in the METAVIR, the Knodell, the Scheuer, the Ludwig, or the Ishak scoring system.

Secondary, or indirect, indices of liver function can also be used to evaluate the efficacy of treatment with a compound of Formulas VI, VII, VIII, IX, X, XI, XII, XIII, XIV, or XV. Morphometric computerized semi-automated assessment of the quantitative degree of liver fibrosis based upon specific staining of collagen and/or serum markers of liver fibrosis can also be measured as an indication of the efficacy of a subject treatment method. Secondary indices of liver function include, but are not limited to, serum transaminase levels, prothrombin time, bilirubin, platelet count, portal pressure, albumin level, and assessment of the Child-Pugh score.

An effective amount of a compound of Formulas VI, VII, VIII, IX, X, XI, XII, XIII, XIV, or XV, and optionally one or more additional antiviral agents, is an amount that is effective to increase an index of liver function by at least about 10%, at least about 20%, at least about 25%, at least about 30%, at least about 35%, at least about 40%, at least about 45%, at least about 50%, at least about 55%, at least about 60%, at least about 65%, at least about 70%, at least about 75%, or at least about 80%, or more, compared to the index of liver function in an untreated individual, or to a placebo-treated individual. Those skilled in the art can readily measure such indices of liver function, using standard assay methods, many of which are commercially available, and are used routinely in clinical settings.

Serum markers of liver fibrosis can also be measured as an indication of the efficacy of a subject treatment method. Serum markers of liver fibrosis include, but are not limited to, hyaluronate, N-terminal procollagen III peptide, 7S domain of type IV collagen, C-terminal procollagen I peptide, and laminin. Additional biochemical markers of liver fibrosis include α-2-macroglobulin, haptoglobin, gamma globulin, apolipoprotein A, and gamma glutamyl transpeptidase.

A therapeutically effective amount of a compound of Formulas VI, VII, VIII, IX, X, XI, XII, XIII, XIV, or XV, and optionally one or more additional antiviral agents, is an amount that is effective to reduce a serum level of a marker of liver fibrosis by at least about 10%, at least about 20%, at least about 25%, at least about 30%, at least about 35%, at least about 40%, at least about 45%, at least about 50%, at least about 55%, at least about 60%, at least about 65%, at least about 70%, at least about 75%, or at least about 80%, or more, compared to the level of the marker in an untreated individual, or to a placebo-treated individual. Those skilled in the art can readily measure such serum markers of liver fibrosis, using standard assay methods, many of which are commercially available, and are used routinely in clinical settings. Methods of measuring serum markers include immunological-based methods, e.g., enzyme-linked immunosorbent assays (ELISA), radioimmunoassays, and the like, using antibody specific for a given serum marker.

As used herein, a “complication associated with cirrhosis of the liver” refers to a disorder that is a sequellae of decompensated liver disease, i.e., or occurs subsequently to and as a result of development of liver fibrosis, and includes, but it not limited to, development of ascites, variceal bleeding, portal hypertension, jaundice, progressive liver insufficiency, encephalopathy, hepatocellular carcinoma, liver failure requiring liver transplantation, and liver-related mortality.

A therapeutically effective amount of a compound of Formulas VI, VII, VIII, IX, X, XI, XII, XIII, XIV, or XV, and optionally one or more additional antiviral agents, is an amount that is effective in reducing the incidence (e.g., the likelihood that an individual will develop) of a disorder associated with cirrhosis of the liver by at least about 10%, at least about 20%, at least about 25%, at least about 30%, at least about 35%, at least about 40%, at least about 45%, at least about 50%, at least about 55%, at least about 60%, at least about 65%, at least about 70%, at least about 75%, or at least about 80%, or more, compared to an untreated individual, or to a placebo-treated individual.

Whether treatment with a compound of Formulas VI, VII, VIII, IX, X, XI, XII, XIII, XIV, or XV, and optionally one or more additional antiviral agents, is effective in reducing the incidence of a disorder associated with cirrhosis of the liver can readily be determined by those skilled in the art.

Reduction in liver fibrosis can increase liver function. Thus, the embodiments provide methods for increasing liver function, generally involving administering a therapeutically effective amount of a compound of Formulas VI, VII, VIII, IX, X, XI, XII, XIII, XIV, or XV, and optionally one or more additional antiviral agents. Liver functions include, but are not limited to, synthesis of proteins such as serum proteins (e.g., albumin, clotting factors, alkaline phosphatase, aminotransferases (e.g., alanine transaminase, aspartate transaminase), 5′-nucleosidase, γ-glutaminyltranspeptidase, etc.), synthesis of bilirubin, synthesis of cholesterol, and synthesis of bile acids; a liver metabolic function, including, but not limited to, carbohydrate metabolism, amino acid and ammonia metabolism, hormone metabolism, and lipid metabolism; detoxification of exogenous drugs; a hemodynamic function, including splanchnic and portal hemodynamics; and the like.

Whether a liver function is increased is readily ascertainable by those skilled in the art, using well-established tests of liver function. Thus, synthesis of markers of liver function such as albumin, alkaline phosphatase, alanine transaminase, aspartate transaminase, bilirubin, and the like, can be assessed by measuring the level of these markers in the serum, using standard immunological and enzymatic assays. Splanchnic circulation and portal hemodynamics can be measured by portal wedge pressure and/or resistance using standard methods. Metabolic functions can be measured by measuring the level of ammonia in the serum.

Whether serum proteins normally secreted by the liver are in the normal range can be determined by measuring the levels of such proteins, using standard immunological and enzymatic assays. Those skilled in the art know the normal ranges for such serum proteins. The following are non-limiting examples. The normal level of alanine transaminase is about 45 IU per milliliter of serum. The normal range of aspartate transaminase is from about 5 to about 40 units per liter of serum. Bilirubin is measured using standard assays. Normal bilirubin levels are usually less than about 1.2 mg/dL. Serum albumin levels are measured using standard assays. Normal levels of serum albumin are in the range of from about 35 to about 55 g/L. Prolongation of prothrombin time is measured using standard assays. Normal prothrombin time is less than about 4 seconds longer than control.

A therapeutically effective amount of a compound of Formulas VI, VII, VIII, IX, X, XI, XII, XIII, XIV, or XV, and optionally one or more additional antiviral agents, is one that is effective to increase liver function by at least about 10%, at least about 20%, at least about 30%, at least about 40%, at least about 50%, at least about 60%, at least about 70%, at least about 80%, or more. For example, a therapeutically effective amount of a compound of Formulas VI, VII, VIII, IX, X, XI, XII, XIII, XIV, or XV, and optionally one or more additional antiviral agents, is an amount effective to reduce an elevated level of a serum marker of liver function by at least about 10%, at least about 20%, at least about 30%, at least about 40%, at least about 50%, at least about 60%, at least about 70%, at least about 80%, or more, or to reduce the level of the serum marker of liver function to within a normal range. A therapeutically effective amount of a compound of Formulas VI, VII, VIII, IX, X, XI, XII, XIII, XIV, or XV, and optionally one or more additional antiviral agents, is also an amount effective to increase a reduced level of a serum marker of liver function by at least about 10%, at least about 20%, at least about 30%, at least about 40%, at least about 50%, at least about 60%, at least about 70%, at least about 80%, or more, or to increase the level of the serum marker of liver function to within a normal range.

### Dosages, Formulations, and Routes of Administration

In the subject methods, the active agent(s) (e.g., compound of Formulas VI, VII, VIII, IX, X, XI, XII, XIII, XIV, or XV, and optionally one or more additional antiviral agents) may be administered to the host using any convenient means capable of resulting in the desired therapeutic effect. Thus, the agent can be incorporated into a variety of formulations for therapeutic administration. More particularly, the agents of the embodiments can be formulated into pharmaceutical compositions by combination with appropriate, pharmaceutically acceptable carriers or diluents, and may be formulated into preparations in solid, semi-solid, liquid or gaseous forms, such as tablets, capsules, powders, granules, ointments, solutions, suppositories, injections, inhalants and aerosols.

### Other Antiviral or Antifibrotic Agents

As discussed above, a subject method will in some embodiments be carried out by administering a compound of Formulas VI, VII, VIII, IX, X, XI, XII, XIII, XIV, or XV, and optionally one or more additional antiviral agent(s).

In some embodiments, the method further includes administration of one or more interferon receptor agonist(s)

In other embodiments, the method further includes administration of pirfenidone or a pirfenidone analog.

Additional antiviral agents that are suitable for use in combination therapy include, but are not limited to, nucleotide and nucleoside analogs. Non-limiting examples include azidothymidine (AZT) (zidovudine), and analogs and derivatives thereof; 2′,3′-dideoxyinosine (DDI) (didanosine), and analogs and derivatives thereof; 2′,3′-dideoxycytidine (DDC) (dideoxycytidine), and analogs and derivatives thereof; 2′,3′-didehydro-2′,3′-dideoxythymidine (D4T) (stavudine), and analogs and derivatives thereof; combivir; abacavir; adefovir dipoxil; cidofovir; ribavirin; ribavirin analogs; and the like.

In some embodiments, the method further includes administration of ribavirin. Ribavirin, 1-β-D-ribofuranosyl-1H-1,2,4-triazole-3-carboxamide, available from ICN Pharmaceuticals, Inc., Costa Mesa, Calif., is described in the Merck Index, compound No. 8199, Eleventh Edition. Its manufacture and formulation is described in U.S. Pat. No. 4,211,771. Some embodiments also involve use of derivatives of ribavirin (see, e.g., U.S. Pat. No. 6,277,830). The ribavirin may be administered orally in capsule or tablet form, or in the same or different administration form and in the same or different route as the subject compound. Of course, other types of administration of both medicaments, as they become available are contemplated, such as by nasal spray, transdermally, intravenously, by suppository, by sustained release dosage form, etc. Any form of administration will work so long as the proper dosages are delivered without destroying the active ingredient.

In some embodiments, the method further includes administration of ritonavir. Ritonavir, 10-hydroxy-2-methyl-5-(1-methylethyl)-1-[2-(1-methylethyl)-4-thiazolyl]-3,6-dioxo-8,11-bis(phenylmethyl)-2,4,7,12-tetraazatridecan-13-oic acid, 5-thiazolylmethyl ester [5S-(5R*,8R*,10R*,11R*)], available from Abbott Laboratories, is an inhibitor of the protease of the human immunodeficiency virus and also of the cytochrome P450 3A and P450 2D6 liver enzymes frequently involved in hepatic metabolism of therapeutic molecules in man.

In some embodiments, the method further includes administration of a protease inhibitor. In some embodiments, the method further includes administration of an NS5A inhibitor. In some embodiments, the method further includes administration of a helicase inhibitor. In some embodiments, the method further includes administration of a polymerase inhibitor.

In some embodiments, an additional antiviral agent is administered during the entire course of the subject compound treatment. In other embodiments, an additional antiviral agent is administered for a period of time that is overlapping with that of the subject compound treatment, e.g., the additional antiviral agent treatment can begin before the subject compound treatment begins and end before the subject compound treatment ends; the additional antiviral agent treatment can begin after the subject compound treatment begins and end after the subject compound treatment ends; the additional antiviral agent treatment can begin after the subject compound treatment begins and end before the subject compound treatment ends; or the additional antiviral agent treatment can begin before the subject compound treatment begins and end after the subject compound treatment ends.

### Methods of Treatment

### Monotherapies

The compounds as described herein may be used in acute or chronic therapy for HCV disease. In many embodiments, the compounds as described herein can be administered for a period of about 1 day to about 7 days, or about 1 week to about 2 weeks, or about 2 weeks to about 3 weeks, or about 3 weeks to about 4 weeks, or about 1 month to about 2 months, or about 3 months to about 4 months, or about 4 months to about 6 months, or about 6 months to about 8 months, or about 8 months to about 12 months, or at least one year, and may be administered over longer periods of time. The compounds as described herein can be administered 5 times per day, 4 times per day, tid, bid, qd, qod, biw, tiw, qw, qow, three times per month, or once monthly. In other embodiments, the NS5A inhibitor compound is administered as a continuous infusion.

In many embodiments, an compounds as described herein of the embodiments can be administered orally.

In connection with the above-described methods for the treatment of HCV disease in a patient, a compound as described herein may be administered to the patient at a dosage from about 0.01 mg to about 100 mg/kg patient bodyweight per day, in 1 to 5 divided doses per day. In some embodiments, the compounds as described herein can be administered at a dosage of about 0.5 mg to about 75 mg/kg patient bodyweight per day, in 1 to 5 divided doses per day.

The amount of active ingredient that may be combined with carrier materials to produce a dosage form can vary depending on the host to be treated and the particular mode of administration. A typical pharmaceutical preparation can contain from about 5% to about 95% active ingredient (w/w). In other embodiments, the pharmaceutical preparation can contain from about 20% to about 80% active ingredient.

Those of skill will readily appreciate that dose levels can vary as a function of the specific compound, the severity of the symptoms and the susceptibility of the subject to side effects. Preferred dosages for a given compound as described herein can be readily determinable by those of skill in the art by a variety of means. A preferred means can be to measure the physiological potency of a given interferon receptor agonist.

In many embodiments, multiple doses of NS5A inhibitor compound are administered. For example, an NS5A inhibitor compound is administered once per month, twice per month, three times per month, every other week (qow), once per week (qw), twice per week (biw), three times per week (tiw), four times per week, five times per week, six times per week, every other day (qod), daily (qd), twice a day (qid), or three times a day (tid), over a period of time ranging from about one day to about one week, from about two weeks to about four weeks, from about one month to about two months, from about two months to about four months, from about four months to about six months, from about six months to about eight months, from about eight months to about 1 year, from about 1 year to about 2 years, or from about 2 years to about 4 years, or more.

Combination Therapies with a TNF-α Antagonist and an Interferon

Some embodiments provide a method of treating an HCV infection in an individual having an HCV infection, the method comprising administering an effective amount of one of the compounds as described herein, and effective amount of a TNF-α antagonist, and an effective amount of one or more interferons.

### Subjects Suitable for Treatment

In certain embodiments, the specific regimen of drug therapy used in treatment of the HCV patient is selected according to certain disease parameters exhibited by the patient, such as the initial viral load, genotype of the HCV infection in the patient, liver histology and/or stage of liver fibrosis in the patient.

Any of the above treatment regimens can be administered to individuals who have been diagnosed with an HCV infection. Any of the above treatment regimens can be administered to individuals having advanced or severe stage liver fibrosis as measured by a Knodell score of 3 or 4 or no or early stage liver fibrosis as measured by a Knodell score of 0, 1, or 2. Any of the above treatment regimens can be administered to individuals who have failed previous treatment for HCV infection (“treatment failure patients,” including non-responders and relapsers).

Individuals who have been clinically diagnosed as infected with HCV are of particular interest in many embodiments. Individuals who are infected with HCV are identified as having HCV RNA in their blood, and/or having anti-HCV antibody in their serum. Such individuals include anti-HCV ELISA-positive individuals, and individuals with a positive recombinant immunoblot assay (RIBA). Such individuals may also, but need not, have elevated serum ALT levels.

Individuals who are clinically diagnosed as infected with HCV include naïve individuals (e.g., individuals not previously treated for HCV, particularly those who have not previously received IFN-α-based and/or ribavirin-based therapy) and individuals who have failed prior treatment for HCV (“treatment failure” patients). Treatment failure patients include non-responders (i.e., individuals in whom the HCV titer was not significantly or sufficiently reduced by a previous treatment for HCV, e.g., a previous IFN-α monotherapy, a previous IFN-α and ribavirin combination therapy, or a previous pegylated IFN-α and ribavirin combination therapy); and relapsers (i.e., individuals who were previously treated for HCV, e.g., who received a previous IFN-α monotherapy, a previous IFN-α and ribavirin combination therapy, or a previous pegylated IFN-α and ribavirin combination therapy, whose HCV titer decreased, and subsequently increased).

In particular embodiments of interest, individuals have an HCV titer of at least about 10⁵, at least about 5×10⁵, or at least about 10⁶, or at least about 2×10⁶, genome copies of HCV per milliliter of serum. The patient may be infected with any HCV genotype (genotype 1, including 1a and 1b, 2, 3, 4, 6, etc. and subtypes (e.g., 2a, 2b, 3a, etc.)), particularly a difficult to treat genotype such as HCV genotype 1 and particular HCV subtypes and quasispecies.

Also of interest are HCV-positive individuals (as described above) who exhibit severe fibrosis or early cirrhosis (non-decompensated, Child's-Pugh class A or less), or more advanced cirrhosis (decompensated, Child's-Pugh class B or C) due to chronic HCV infection and who are viremic despite prior anti-viral treatment with IFN-α-based therapies or who cannot tolerate IFN-α-based therapies, or who have a contraindication to such therapies. In particular embodiments of interest, HCV-positive individuals with stage 3 or 4 liver fibrosis according to the METAVIR scoring system are suitable for treatment with the methods described herein. In other embodiments, individuals suitable for treatment with the methods of the embodiments are patients with decompensated cirrhosis with clinical manifestations, including patients with far-advanced liver cirrhosis, including those awaiting liver transplantation. In still other embodiments, individuals suitable for treatment with the methods described herein include patients with milder degrees of fibrosis including those with early fibrosis (stages 1 and 2 in the METAVIR, Ludwig, and Scheuer scoring systems; or stages 1, 2, or 3 in the Ishak scoring system.).

### Synthesis

The compounds and processes of the present disclosure will be better understood in connection with the following synthetic schemes which illustrate the methods by which the compounds of the present disclosure may be prepared. Starting materials can be obtained from commercial sources or prepared by well-established literature methods known to those of ordinary skill in the art. The variables are as defined above unless otherwise noted below.

### Section I

### Preparation of Compounds: Section I

### Example I

### Preparation of General Compound 101

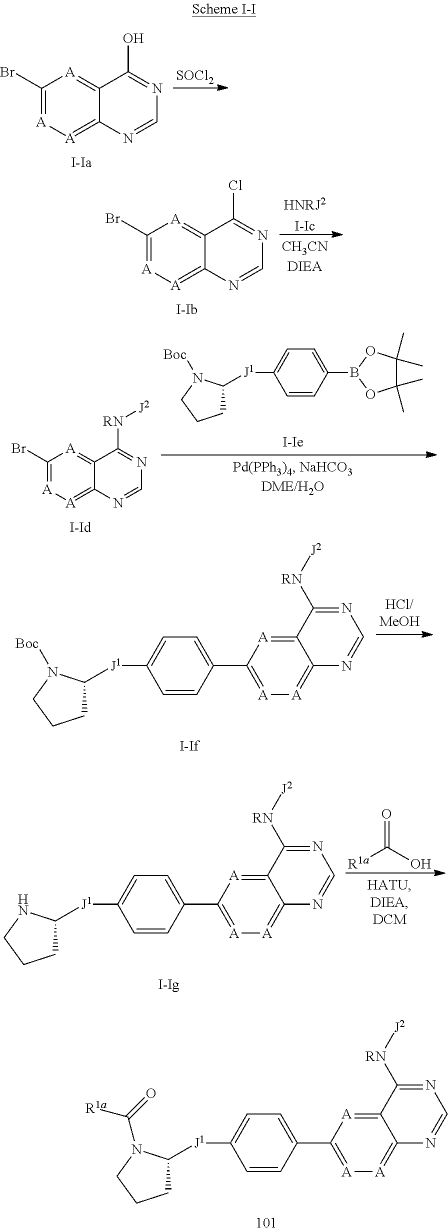

The variables shown in the Schemes of Example I are defined according to the definitions for Formula XI.

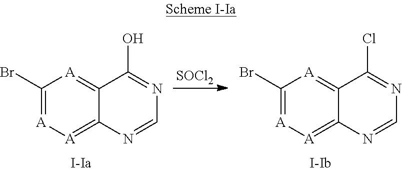

### General Procedure I-A

To a solution of general compound I-Ia (4.4 mmol) in SOCl₂ (10 mL) is added DMF (0.3 mL) and the reaction mixture is stirred at reflux overnight. After cooling to room temperature, the mixture is concentrated under reduced pressure to afford general compound I-Ib, which is used in the next step without further purification.

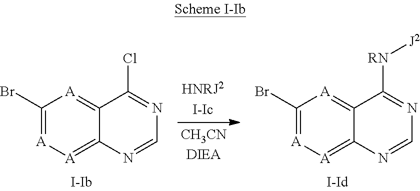

### General Procedure I-B

To a mixture of general compound I-Ib (4.5 mmol), general compound I-Ic (5.0 mmol) in CH₃CN (15 mL) is added DIEA (6.2 mmol). The reaction mixture is stirred at reflux for two days. After cooling down to room temperature, the precipitate solid is collected by filtration. The solid is washed with CH₃CN twice to afford general compound I-Id.

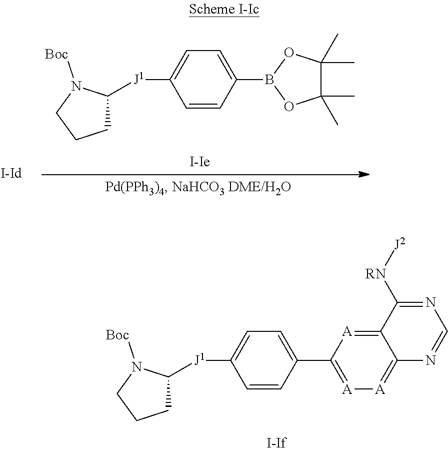

### General Procedure I-C

To a mixture of general compound I-Id (1.0 mmol), general compound I-Ie (1.3 mmol), NaHCO₃ (0.17 g, 2.0 mmol) in DME/H₂O (9 mL/3 mL) is added Pd(PPh₃)₄ (0.04 g, catalyzed amount) under N₂ protection. The resulting mixture is stirred at reflux for 5 hours, and then cooled to room temperature. Subsequently, H₂O (20 mL) is added, and the mixture is extracted with EtOAc (20 mL×3). The combined organic layers are washed with brine (12 mL×2), dried over anhydrous Na₂SO₄, filtered and the filtrate is concentrated. The residue is purified by column chromatography on silica gel to afford general compound I-If (0.5 g, yield 83%).

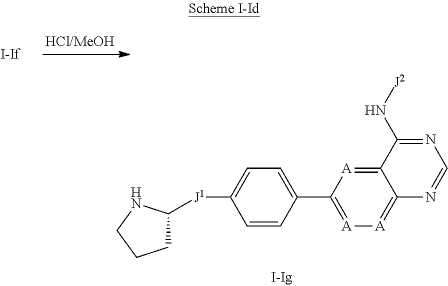

### General Procedure I-D

A mixture of general compound I-If (0.3 mmol) in HCl/MeOH (5 mL) is stirred at room temperature for 1 hour. The solvent is removed under reduced pressure to afford general compound I-Ig.

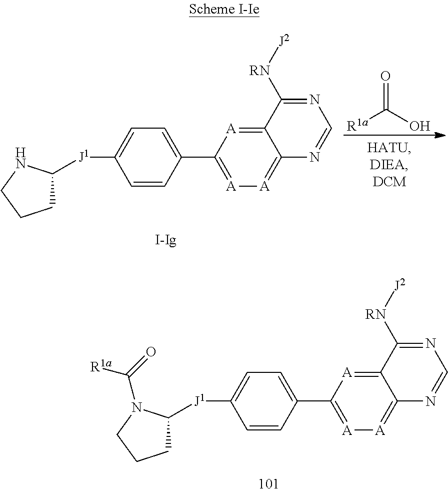

### General Procedure I-E

To a mixture of general compound I-Ig (0.32 mmol), general compound I-Ig (0.4 mmol), HATU (0.2 g, 0.5 mmol) in DCM (5 mL) was added DIEA (0.4 g, 1.6 mmol). The resulting mixture is stirred at room temperature for 1 hour. Subsequently, the mixture is treated with water (10 mL) and extracted with DCM (15 mL×3). The combined organic layer is dried over Na₂SO₄ and concentrated under reduced pressure. The crude product is purified by Prep-HPLC to afford general compound 101.

### Example VIII-XVI

### Preparation of Compound 421

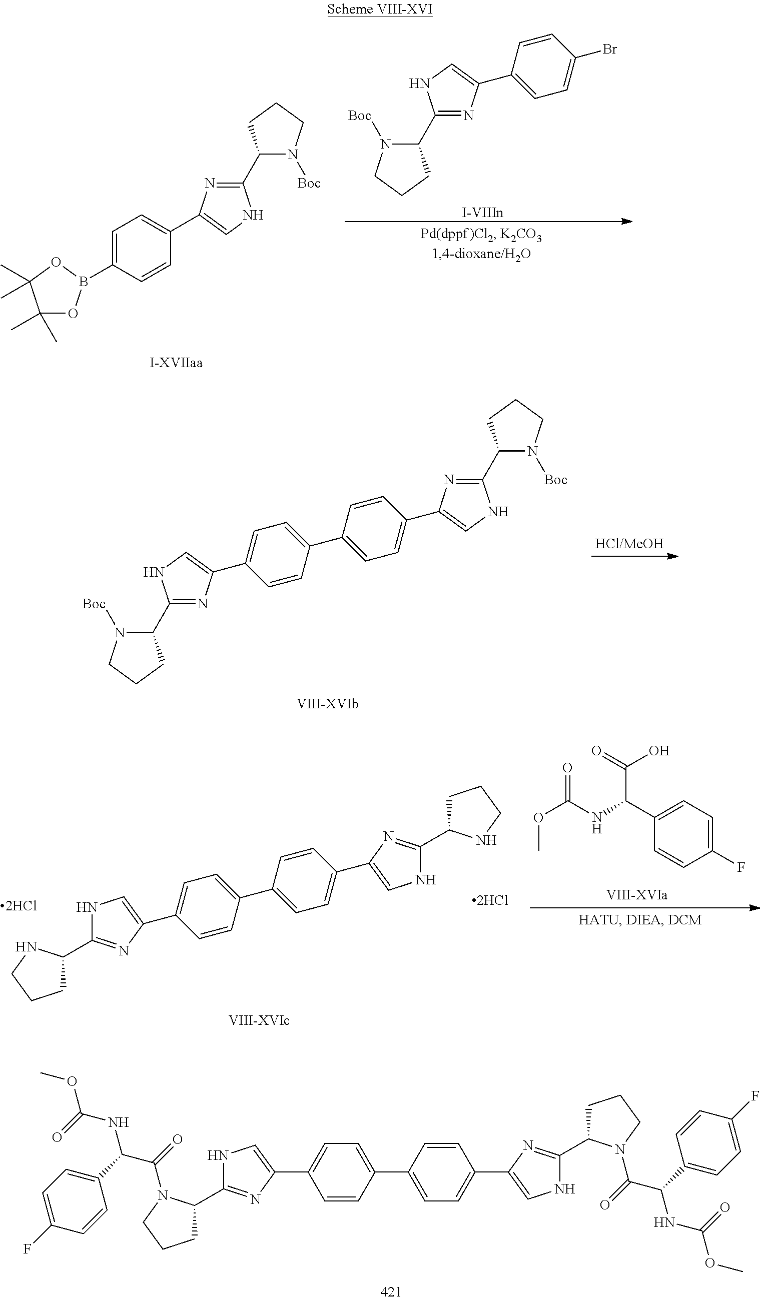

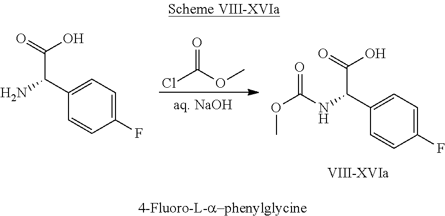

### General Procedure VIII-BT

A solution of 4-Fluoro-L-α-phenylglycine (1.7 g, 10 mmol) in 10 mL of 1N sodium hydroxide solution was cooled to 10° C. Methyl chloroformate (0.94 g, 10 mmol) and 10 mL of a 2N NaOH solution was added simultaneously. After stirring for 16 hrs at r.t., the mixture was acidified with 1N aqueous HCl until the pH reached to 2, and the product was extracted with ethyl acetate (100 mL×3). The extracts were combined, dried over sodium sulfate, filtered and concentrated to yield compound VIII-XVIa (0.9 g, yield 39%) as a white solid.

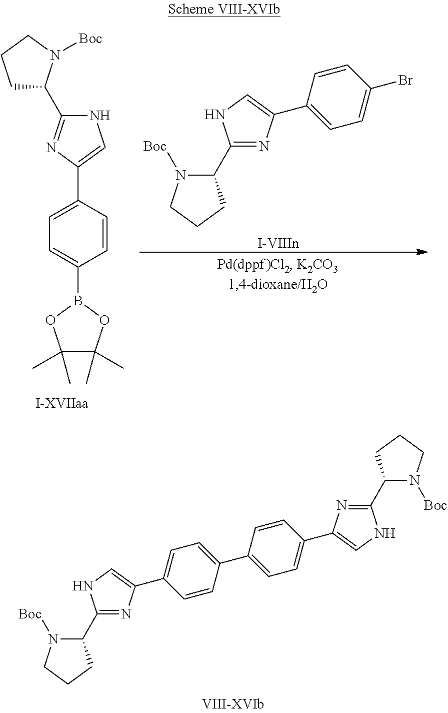

### General Procedure VIII-BU

To a mixture of compound I-XVIIaa (1.0 g, 2.2 mmol), compound I-VIIIn (0.89 g, 2.2 mmol), K₂CO₃ (0.62 g, 4.5 mmol) in dioxane/H₂O (20 mL/1 mL) was added Pd(dppf)Cl₂ (0.1 g, catalyzed amount) under N₂. The reaction mixture was refluxed overnight under N₂. After being cooled to room temperature, the reaction mixture was diluted with H₂O (20 mL), extracted with EtOAc (20 mL×3). The organic layers were combined, washed with brine (30 mL), dried over anhydrous Na₂SO₄, filtered and the filtrate was concentrated in vacuo. The residue was purified by column chromatography on silica gel to obtain compound VIII-XVIb (0.8 g, yield 57%).

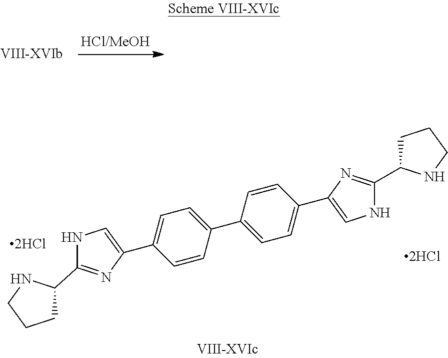

### General Procedure VIII-BV

The mixture of compound VIII-XVIb (0.1 g, 0.16 mmol) in HCl/MeOH (4 M, 3 mL) was stirred at room temperature for 1 hour. After being concentrated under reduced pressure, compound VIII-XVII was used in the next step without further purification.

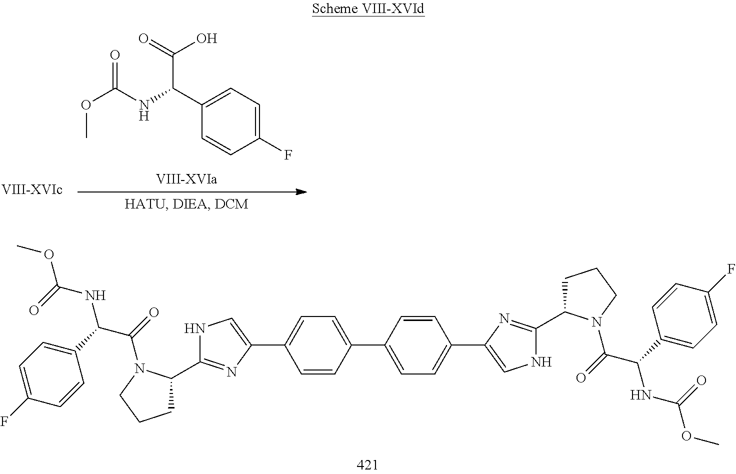

### General Procedure VIII-BW

To a mixture of compound VIII-XVIc (0.1 g, 0.24 mmol), compound VIII-XVIa (0.11 g, 0.48 mmol), HATU (0.2 g, 0.52 mmol) in DCM (5 mL) was added DIEA (0.18 g, 1.4 mmol). The resulting mixture was stirred at room temperature for 2 hrs. And then the mixture was diluted with water (10 mL) and extracted with DCM (15 mL×3). The combined organic layers was dried over Na₂SO₄ and concentrated under reduced pressure. The residue was purified by Prep-HPLC to afford compound 421 (12 mg, yield 6%) as a yellow solid. ¹H NMR (300 MHz, CDCl₃): δ 7.82-7.64 (m, 2H), 7.61-7.49 (m, 6H), 7.43-7.30 (m, 4H), 7.29-7.04 (m, 3H), 7.00-6.92 (m, 3H), 6.16 (d, J=6.0 Hz, 2H), 5.50 (d, J=8.1 Hz, 2H), 5.30 (br, 2H), 3.74 (br, 2H), 3.68 (s, 6H), 3.32 (br, 2H), 2.80 (br, 2H), 2.25-1.88 (m, 8H). MS (ESI) m/z (M+H)⁺ 843.2.

### Example VIII-XVII

### Preparation of Compound 422

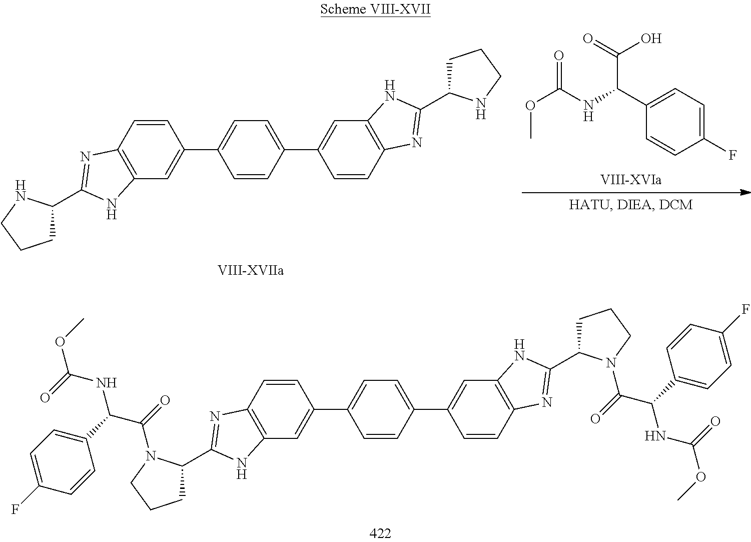

### General Procedure VIII-BX

To a mixture of compound VIII-XVIIa (0.1 g, 0.22 mmol), compound VIII-XVIa (0.11 g, 0.48 mmol), HATU (0.19 g, 0.5 mmol) in DCM (5 mL) was added DIEA (0.17 g, 1.3 mmol). The resulting mixture was stirred at room temperature for 2 hrs. The mixture was then diluted with water (10 mL) and extracted with DCM (15 mL×3). The combined organic layer was dried over Na₂SO₄ and concentrated under reduced pressure. The crude product was purified by Prep-HPLC to yield 422 (10 mg, yield 6%) as a yellow solid. ¹H NMR (400 MHz, CDCl₃): δ 7.69-7.40 (m, 11H), 7.33-7.26 (m, 3H), 7.10-7.05 (m, 1H), 6.97-6.93 (m, 3H), 6.20 (d, J=8.0 Hz, 2H), 5.54-5.45 (m, 4H), 3.73-3.64 (m, 8H), 3.35 (br, 1H), 2.86 (br, 2H), 2.31-1.96 (m, 8H). MS (ESI) m/z (M+H)⁺ 867.3.

### Example VIII-XVIII

### Preparation of Compound 423

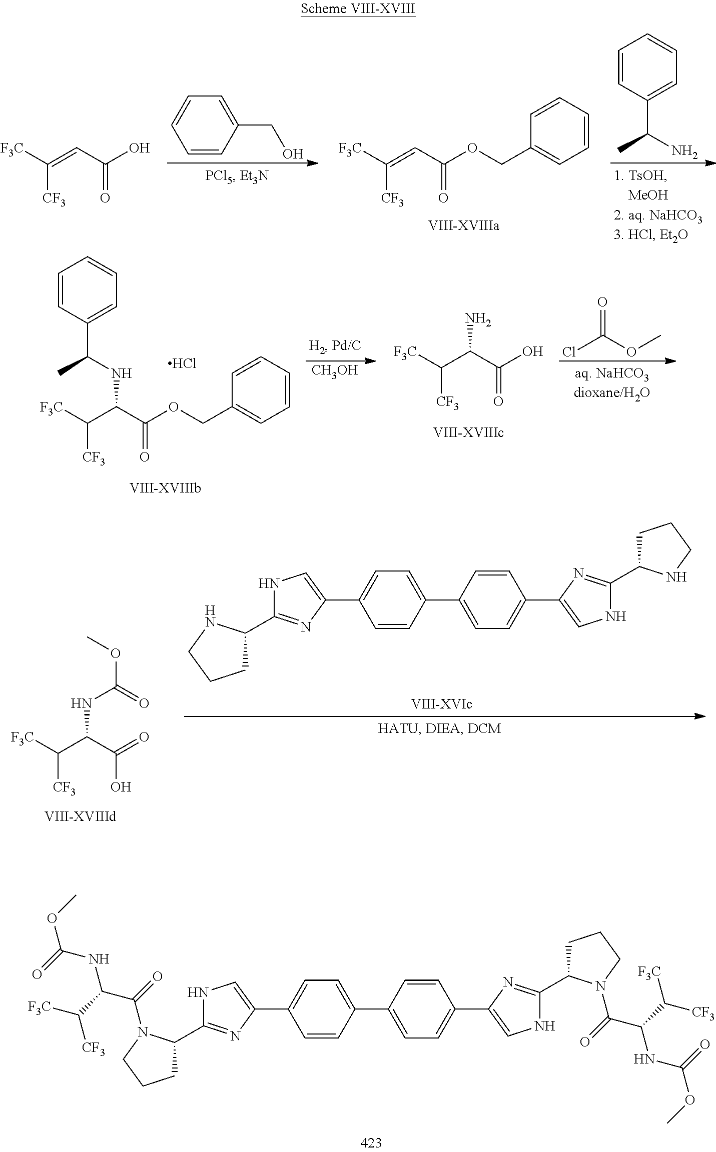

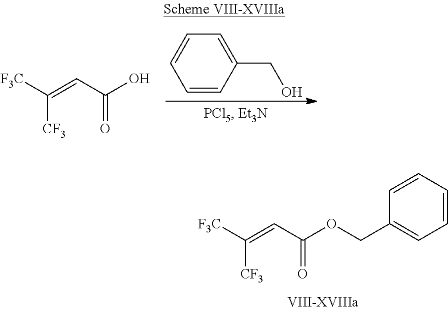

### General Procedure VIII-BY

To a solution of 4,4,4-trifluoro-3-(trifluoromethyl)but-2-enoic acid (5.0 g, 24 mmol) in DCM (50 mL) was added PCl₅ (5 g, 24 mmol) in portions under ice cooling and then the mixture was stirred at room temperature for 3 hours. After concentration under reduced pressure, the residue was dissolved in 10 mL of DCM and added to a solution of benzyl alcohol (2.9 g, 27 mmol), Et₃N (9.7 g, 96 mmol) in DCM (50 mL) at 0° C. The reaction mixture was stirred at room temperature overnight. Water (20 mL) was added and extracted with EtOAc, the organic layers was separated, dried over anhydrous Na₂SO₄ and concentrated, the residue was purified by column chromatography to afford compound VIII-XVIIIa (5.2 g, yield 72%).

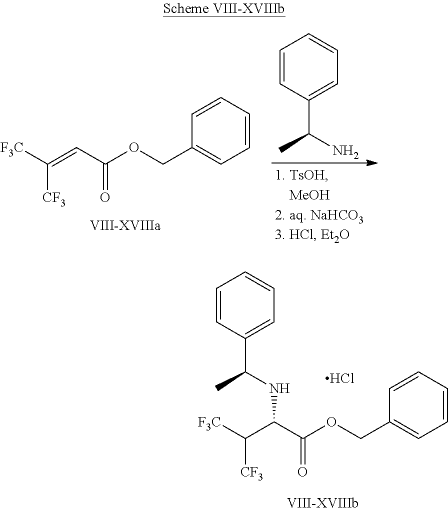

### General Procedure VIII-BZ

To a solution of compound VIII-XVIIIa (2.0 g, 6.7 mmol) in MeOH (10 mL) was added (S)-(−)-α-methylbenzylamine (0.82 g, 6.7 mmol) at −70° C. The mixture was warmed to room temperature and stirred for 1 hour and then treated with TsOH (1.27 g, 7.4 mmol) in MeOH (10 mL). Subsequently, the MeOH was evaporated to remove most of MeOH and then Et₂O was added until the HCl salt began to solidify. The solid was collected by filtration and then added to a mixture of Et₂O (10 mL) and saturated aq. NaHCO₃ (10 mL). After the evolution of CO₂ (gas) had ceased, the mixture was extracted with Et₂O. The organic layers were separated, and the Et₂O solution was dried over Na₂SO₄ and the solid removed by filtration. After filtration, the filtrate was treated with HCl gas for 25 mins, and the solvent removed afford compound VIII-XVIIIb (0.7 g, yield 23%) as a HCl salt.

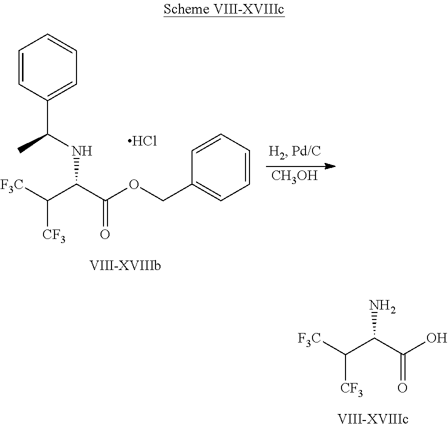

### General Procedure VIII-CA

Pd/C (10%, 0.07 g) was added to a mixture of compound VIII-XVIIIb (0.7 g, 1.5 mmol) in EtOH (10 mL). The mixture was stirred under 50 psi of H₂ at room temperature for 6 hours. Subsequently, the solid was removed by filtration. After filtration, the filtrate was concentrated to afford compound VIII-XVIIIc (0.3 g, yield 86%).

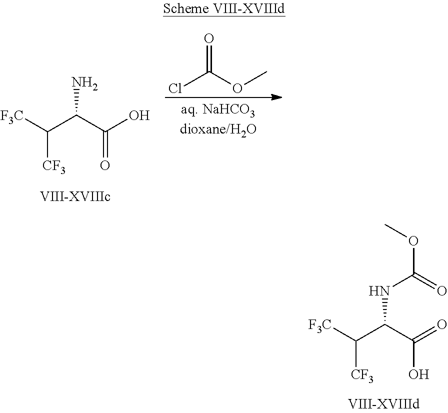

### General Procedure VIII-CB

To a mixture of compound VIII-XVIIIc (0.1 g, 0.4 mmol), NaHCO₃ (1.0 g, 11.9 mmol) in dioxane (2 mL) and H₂O (2 mL) was added methyl chloroformate (0.04 g, 0.4 mmol) at 0° C. Subsequently, the reaction mixture was stirred at room temperature for 5 hours. The mixture was then extracted with EtOAc. The remaining aqueous layers was acidified with diluted HCl to pH=2 and re-extracted with EtOAc. The combined organic layers were dried over Na₂SO₄ and concentrated to afford compound VIII-XVIIIc (0.09 g, yield 69%).

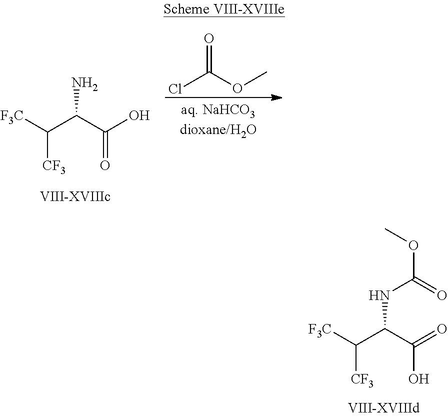

### General Procedure VIII-CC

To a mixture of compound VIII-XVIIIc (0.1 g, 0.4 mmol), NaHCO₃ (1.0 g, 11.9 mmol) in dioxane (2 mL) and H₂O (2 mL) was added methyl chloroformate (0.04 g, 0.4 mmol) at 0° C. Subsequently, the reaction mixture was stirred at room temperature for 5 hours. The mixture was then extracted with EtOAc. The remaining aqueous layers was acidified with diluted HCl to pH=2 and re-extracted with EtOAc. The combined organic layers were dried over Na₂SO₄ and concentrated to afford compound VIII-XVIIId (0.09 g, yield 69%).

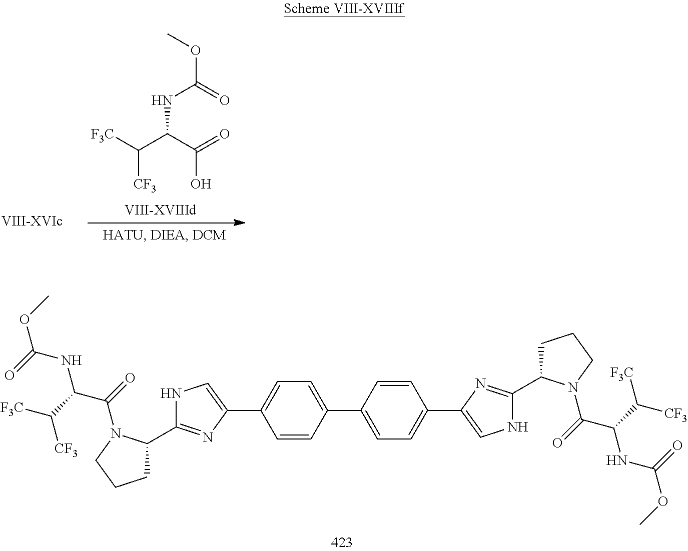

### General Procedure VIII-CC

To a solution of compound VIII-XVIc (0.065 g, 0.2 mmol), compound VIII-XVIIId (0.09 g, 0.3 mmol), DIEA (0.5 g, 3.9 mmol) in CH₂Cl₂ (5 mL) was added HATU (0.2 g, 0.5 mmol). The resulting mixture was stirred at room temperature for 1 hour. Then the mixture was diluted with H₂O and extracted with EtOAc. The organic phase was separated, dried over Na₂SO₄ and concentrated. The residue was purified by prep-HPLC to afford compound 423 (0.02 g, yield 13%). ¹H NMR (400 MHz, CD₃OD) δ 7.68-7.76 (m, 8H), 7.41 (s, 2H), 5.30 (d, J=7.6 Hz, 2H), 5.16-5.27 (m, 2H), 4.29-4.33 (m, 2H), 3.86-3.89 (m, 2H), 3.76 (s, 6H), 2.07-2.34 (m, 10H). MS (ESI) m/z (M+H)⁺ 954.9.

### Example VIII-XIX

### Preparation of Compound 424

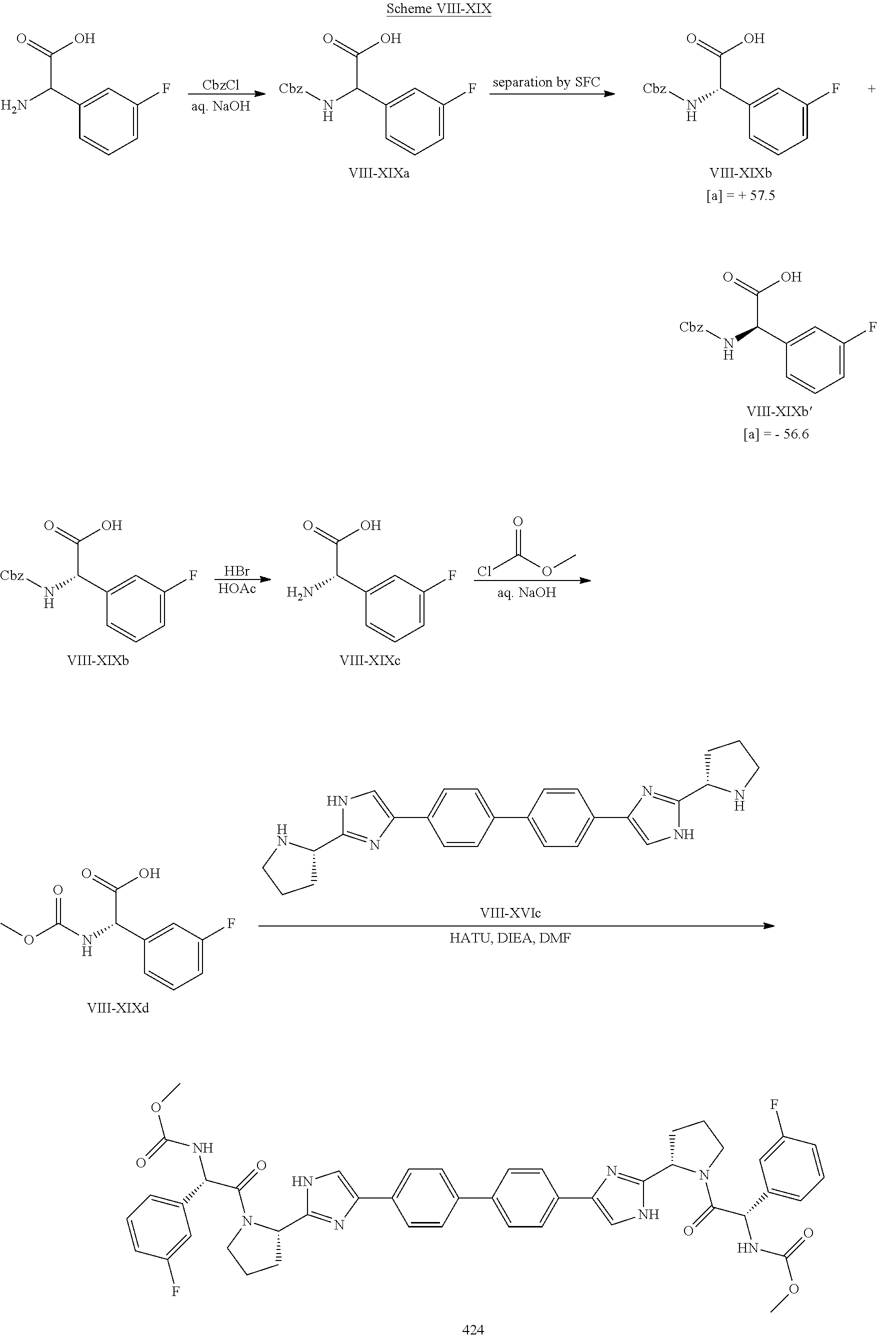

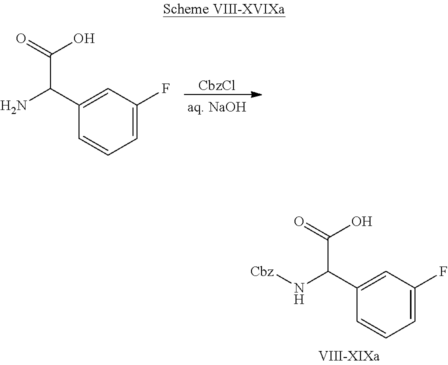

### General Procedure VIII-CD

To a solution of 3-Fluoro-α-phenylglycine (1 g, 5.9 mmol) in 10 mL of NaOH (2M) was added CbzCl (2.5 g, 14.7 mmol) dropwise at 0° C. and the mixture was stirred at room temperature for 3 hrs. The mixture was then acidified to pH 2˜3 with 2N HCl and extracted with EtOAc (20 mL×3), the organic phase was washed with water and brine, dried over anhydrous Na₂SO₄ and concentrated under reduced pressure. After concentration under reduced pressure, the residue was purified by column chromatography to afford compound VIII-XIXa (1.2 g, yield 60%). ¹H NMR (300 MHz, DMSO-d₆): δ 13.01 (s, 1H), 8.18 (d, J=8.4 Hz, 1H), 7.10-7.39 (m, 9H), 5.22 (d, J=6.6 Hz, 1H), 5.04 (s, 2H). Compound VIII-XIXa was separated by supercritical fluid chromatography (SFC) to yield 500 mg of compound 3. Column: CHIRALPAK®AS, 250×4.6 mm I.D, 5 μm.

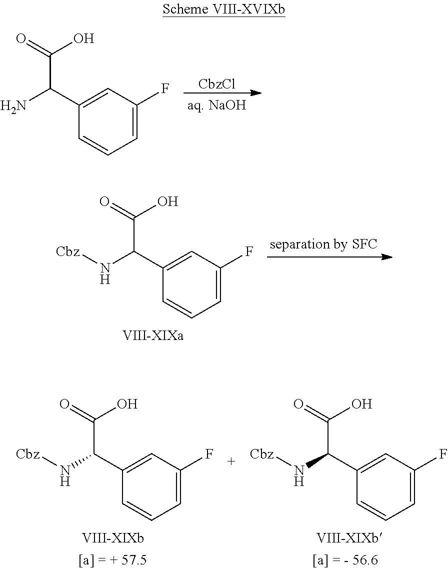

### General Procedure VIII-CE

To a solution of 3-Fluoro-α-phenylglycine (1 g, 5.9 mmol) in 10 mL of NaOH (2M) was added CbzCl (2.5 g, 14.7 mmol) dropwise at 0° C. and the mixture was stirred at room temperature for 3 hrs. The mixture was then acidified to pH 2˜3 with 2N HCl and extracted with EtOAc (20 mL×3), the organic phase was washed with water and brine, dried over anhydrous Na₂SO₄ and concentrated under reduced pressure. After concentration under reduced pressure, the residue was purified by column chromatography to afford compound VIII-XIXa (1.2 g, yield 60%). ¹H NMR (300 MHz, DMSO-d₆): δ 13.01 (s, 1H), 8.18 (d, J=8.4 Hz, 1H), 7.10-7.39 (m, 9H), 5.22 (d, J=6.6 Hz, 1H), 5.04 (s, 2H). Compound VIII-XIXa was separated by supercritical fluid chromatography (SFC) to yield 500 mg of compound XIXb. Column: CHIRALPAK®AS, 250×4.6 mm I.D, 5 μm.

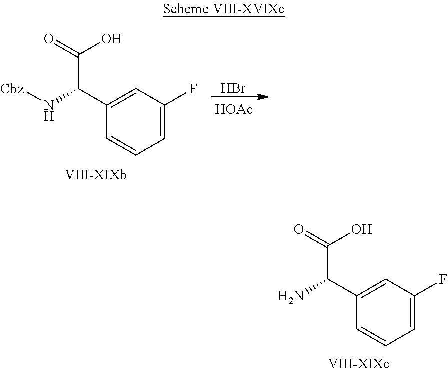

### General Procedure VIII-CF

To a mixture of compound XIXb (500 mg, 1.6 mmol) in HOAc (5 mL) was added HBr (5 mL, 40% in HOAc) dropwise. The resulting mixture was stirred at room temperature for 2 hrs. The mixture was concentrated under reduced pressure to afford 3-fluoro-L-α-phenylglycine (VIII-XIXc, 270 mg, yield 100%), which was used in the next step without further purification.

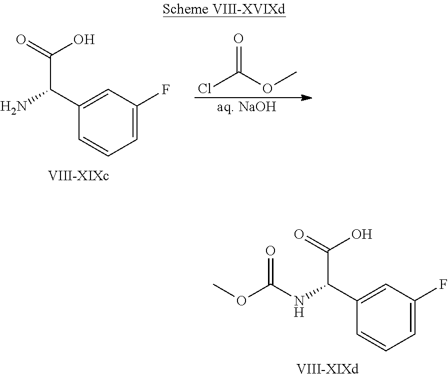

### General Procedure VIII-CG

To a solution of 3-fluoro-L-α-phenylglycine (VIII-XIXc, 270 mg, 1.6 mmol) in 5 mL of H₂O was added NaOH (256 mg, 6.4 mmol) and the mixture was stirred at room temperature for 0.5 hour, then methyl chloroformate (225 mg, 2.4 mmol) was added dropwise and the mixture was stirred at room temperature for additional 2 hrs. Then the mixture was extracted with EtOAc (20 mL). The aqueous phase was separated, acidified to pH 2˜3 with 2N HCl and extracted with EtOAc (20 mL×3), the organic phase was washed with water and brine, dried over anhydrous Na₂SO₄, and concentrated to afford N-methoxycarbonyl-3-fluoro-L-α-phenylglycine (VIII-XIXd, 240 mg, yield 66%). ¹H NMR (300 MHz, CD₃OD): δ 7.01-7.40 (m, 4H), 5.25 (s, 1H), 3.64 (s, 3H).

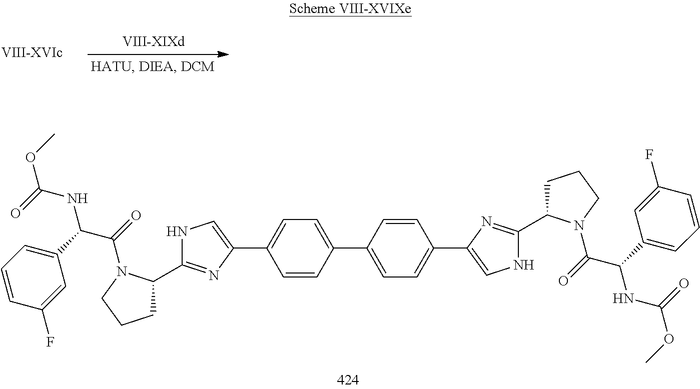

### General Procedure VIII-CH

To a mixture of compound VIII-XVIc (113 mg, 0.27 mmol) in DMF (5 mL) were added DIPEA (104 mg, 0.81 mmol), HATU (205 mg, 0.54 mmol) and compound VIII-XIXd (120 mg, 0.54 mmol). The resulting mixture was stirred at room temperature for 2 hours and then the mixture was diluted with EtOAc (20 mL), the organic layer was washed with water and brine, dried over anhydrous Na₂SO₄ and concentrated under reduced pressure. The residue was purified by Prep-HPLC to afford compound 424 (50 mg, yield 22%). ¹H NMR (300 MHz, CD₃OD): δ 7.63-7.83 (m, 8H), 7.04-7.44 (m, 10H), 5.16-5.25 (m, 2H), 4.93-4.95 (m, 2H), 3.86-3.89 (m, 2H), 3.58 (s, 6H), 3.53 (s, 2H), 2.17-2.33 (m, 5H), 1.62-2.14 (m, 3H). MS (EI) m/z: (M+H)⁺ 843.4.

### Section II

### Preparation of Compounds: Section II

### Example IX-I

### Preparation of Compound 501

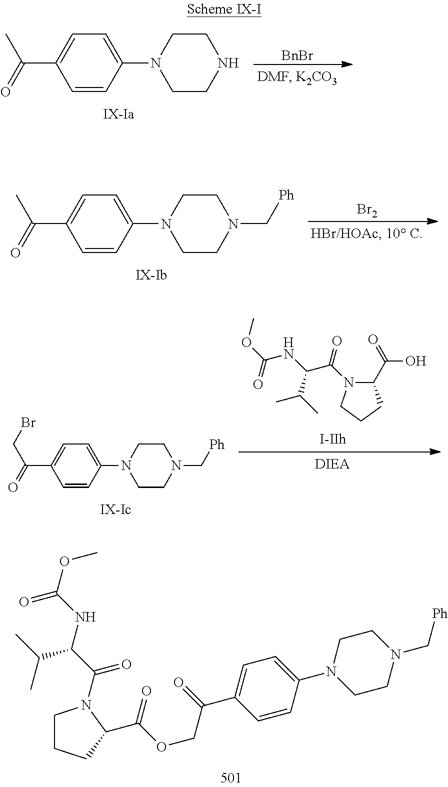

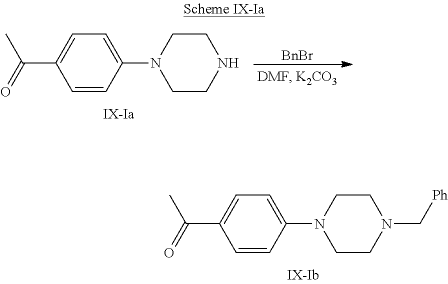

### General Procedure IX-A

A mixture of 1-(4-(piperazin-1-yl)phenyl)ethanone (IX-Ia) (2 g, 9.8 mmol), K₂CO₃ (2.02 g, 14.7 mmol) in DMF (25 mL) was stirred at r.t. for 5 min, followed by addition of a solution of benzyl bromide (1.17 mL, 9.8 mmol) in THF (15 mL). The mixture was stirred at r.t. overnight. The solvent was removed in reduced pressure, and the residue was re-crystallized in EtOAc to give 1-(4-(4-benzylpiperazin-1-yl)phenyl)ethanone (IX-Ib) (2.9 g, yield 99%) as a light yellow solid.

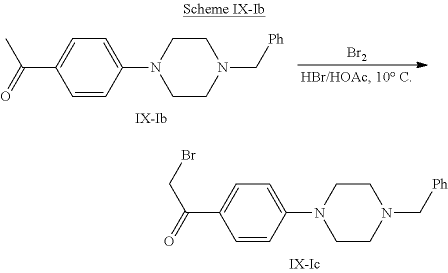

### General Procedure IX-B

To a solution of Br₂ (0.59 g, 3.74 mmol) in HBr/HOAc (20 mL), was added 1-(4-(4-benzylpiperazin-1-yl)phenyl)ethanone (IX-Ib) (1 g, 3.4 mmol) in several portions. The mixture was stirred at r.t. overnight. The mixture was poured into 200 mL of EtOAc. The precipitate was collected by filtration. The solid was dried under vacuum to afford compound IX-Ic (1.2 g, 67%) as a light yellow solid.

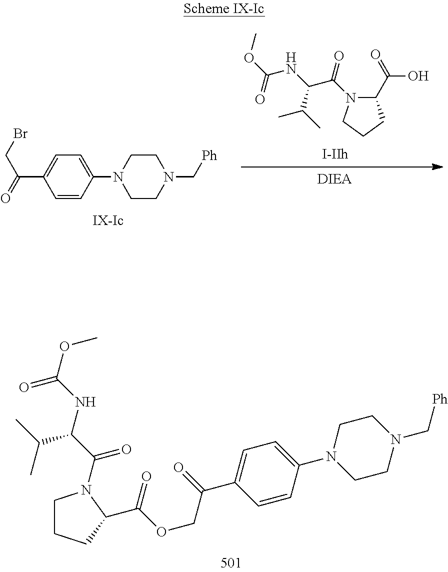

### General Procedure IX-C

A mixture of compound IX-Ic (0.146 g, 0.536 mmol), compound I-IIh (0.1 g, 0.268 mmol), THF (10 mL) and DIEA (0.104 g, 0.804 mmol) was stirred at 25° C. for 72 hours. The solvent was removed, and the residue was purified by prep-TLC to afford 501 (0.1 g, yield 66%) after solvent removal as a white solid. MS (ESI) m/z (M+H)+ 565.1.

### Example IX-II

### Preparation of Compound 502 and 503

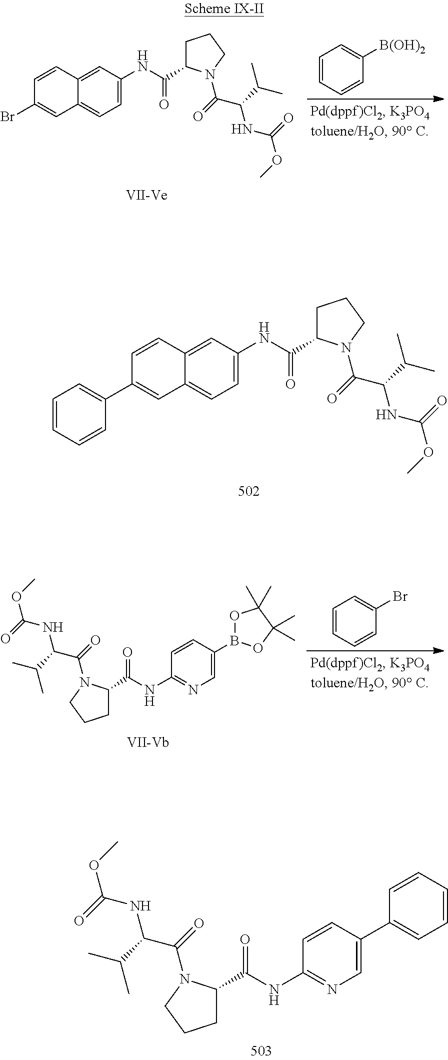

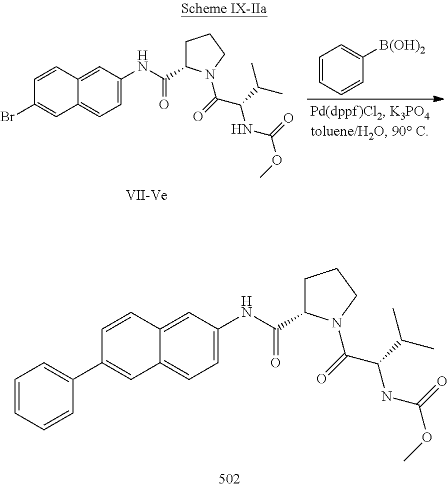

### General Procedure IX-D

General Method VII-V was followed for preparation of compound 502 (10 mg, 19%). MS (ESI) m/z (M+H)⁺ 474.2.

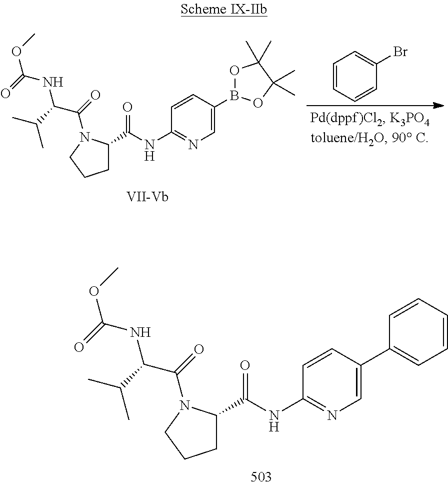

### General Procedure IX-E

General Method VII-V was followed for preparation of compound 503 (9 mg, yield 11%). MS (ESI) m/z (M+Na)⁺ 447.1.

### Example IX-III

### Preparation of Compound 504

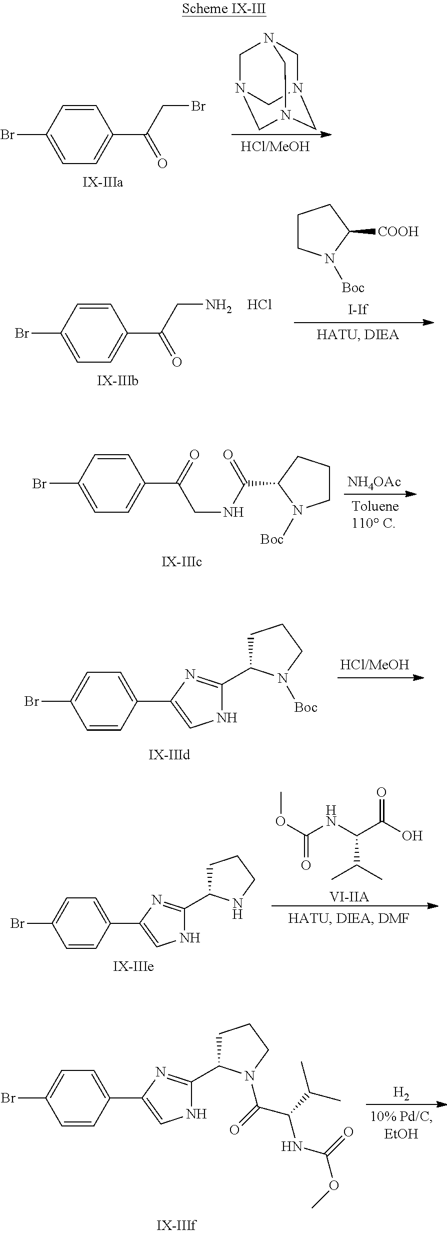

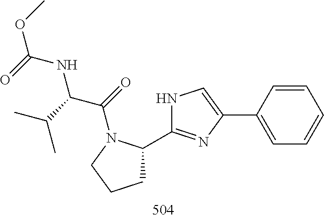

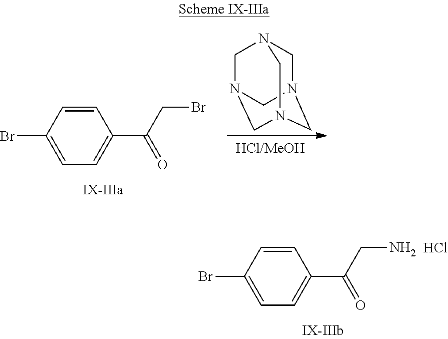

### General Procedure IX-F

A mixture of 2-bromo-1-(4-bromophenyl)ethanone (IX-IIIa) (20 g 7.2 mmol) and hexamethylenetetramine (10.3 g, 72 mmol) in chloroform (400 mL) was stirred at r.t. overnight. The precipitated solid was collected by filtration, washed with chloroform, and dried in vacuo. Then the solid was suspended in a mixture of concentrated HCl (80 mL) and methanol (300 mL). And the suspension was stirred at r.t. overnight. The precipitated solid was collected by filtration, washed with methanol (50 mL) and dried in vacuo to afford 18 g of 2-amino-1-(4-bromophenyl)ethanone hydrochloride (IX-IIIb), which was used without further purification.

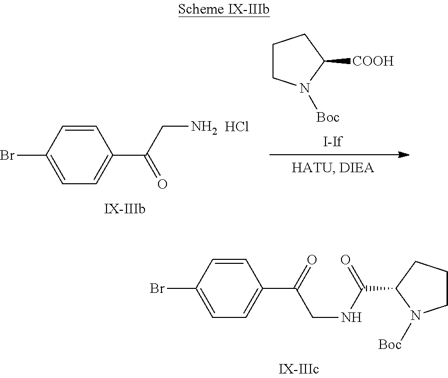

### General Procedure IX-G

HATU (38.2 g, 0.11 mol), diisopropylethylamine (23.8 g, 0.2 mmol) and N-Boc-L-proline (I-If) (14.4 g, 67.2 mmol) were added to a suspension of 2-amino-1-(4-bromophenyl)ethanone hydrochloride (IX-IIIb) (16.0 g, 64.1 mmol) in tetrahydrofuran (120 mL). The resulting mixture was stirred for 4 hrs as the solids dissolved. The reaction mixture was quenched by the addition of 13% aqueous sodium chloride (50 mL) and stirred another 30 mins. The layers were separated, and the organic layer was mixed with toluene (150 mL) and concentrated to a volume of 100 mL. The solution, which contained compound IX-IIIc, was used in the next step.

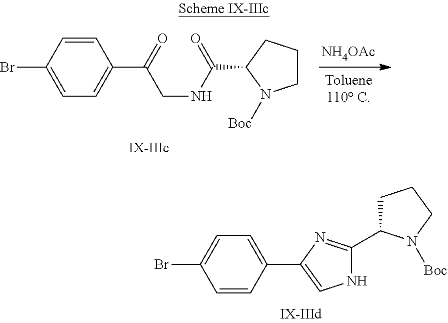

### General Procedure IX-H

The solution of compound IX-IIIc, obtained in the previous experiment, was treated with ammonium acetate (53.7 g, 672 mmol) and heated to 100-110° C. 25 h. The mixture was allowed to cool to room temperature, filtered, concentrated, and the residue obtained was purified by column chromatography (300 g silica gel cartridge, isocratic elution with ethyl acetate/petroleum=1:10-1:2), yielding compound IX-IIId (15.0 g, yield 61% over two steps).

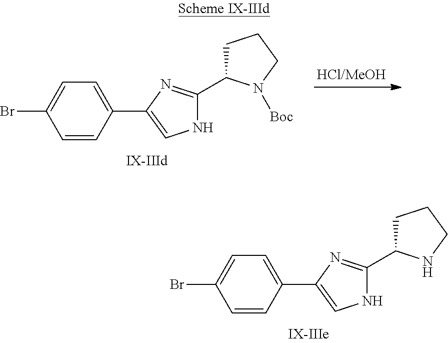

### General Procedure IX-I

Compound IX-IIId (783 mg, 2 mmol) was added into HCl/CH₃OH (40 mL, 4M). Then the mixture was stirred at room temperature for 2-3 hrs. When the reaction completed, the mixture was concentrated in vacuum to afford compound IX-IIIe (600 mg, yield ˜100%).

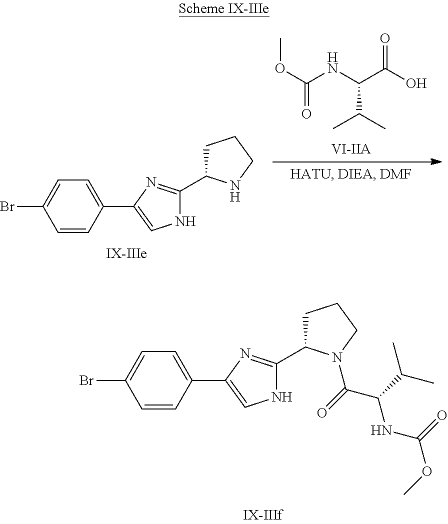

### General Procedure IX-J

To a mixture of compound IX-IIIe (300 mg, 1 mmol), compound VI-IIA (180 mg, 1 mmol) and DIPEA (400 mg, 3 mmol) in DMF (10 mL) was added HATU (470 mg, 1.1 mmol). The resulting mixture was stirred at room temperature. LCMS judged the material 6 was consumed up. The mixture was diluted with EtOAc, and the solution was washed with brine, dried over Na₂SO₄, concentrated. The residue was purified by chromatography on silica gel to afford compound IX-IIIf (300 mg, yield 67%).

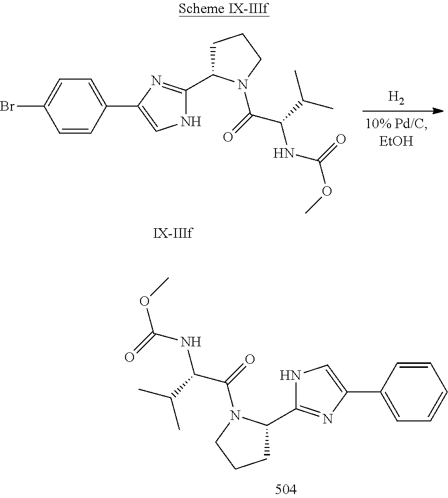

### General Procedure IX-K

To a solution of compound IX-IIIf (50 mg, 0.13 mmol) in EtOH (8 mL) was added Pd—C (10%, 5 mg) under N₂. The suspension was degassed under vacuum and purged with H₂ several times. The mixture was stirred under H₂ (45 psi) at r.t. for 5 hours. TLC showed the reaction was completed. Then Pd/C was filtered off, the filtrate was concentrated under reduced pressure, the residue was purified by Prep. TLC (PE:EA=5:1) to afford 504 (10 mg, yield 21%). MS (ESI) m/z (M+H)⁺ 371.

### Example IX-IV

### Preparation of Compound 505

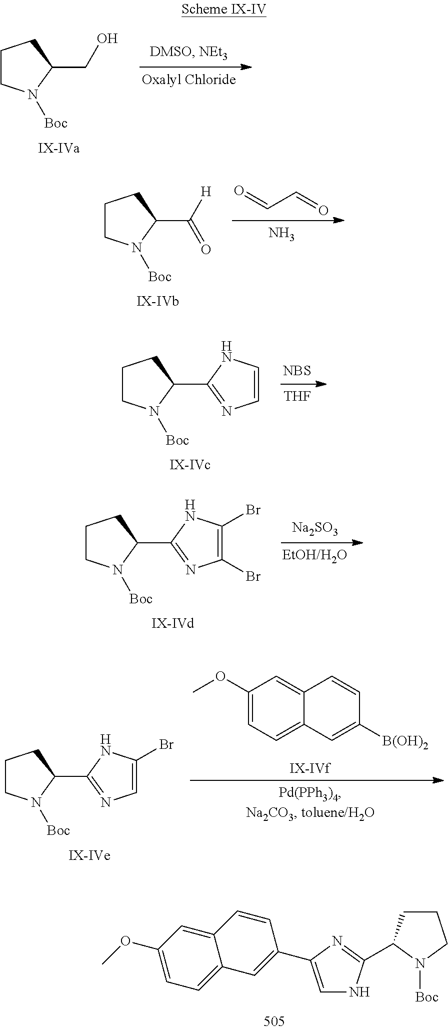

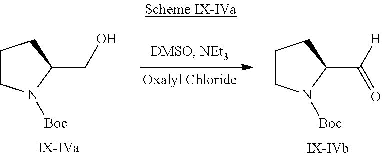

### General Procedure IX-L

A flask containing DMSO (35 mL, 0.5 mol) and CH₂Cl₂ (200 mL) under nitrogen is stirred at −78° C. A solution of oxalyl chloride in CH₂Cl₂ (2M, 35 mL, 0.3 mol) is added dropwise and the reaction is stirred for 1 hour at −78° C. A solution of Boc-prolinol (IX-IVa) (50 g, 0.25 mol) in CH₂Cl₂ (100 mL) was added dropwise and the reaction stirred at −78° C. for 2 hours. Triethylamine (120 mL, 1 mol) was added to the mixture and the reaction allowed to warm to r.t. The reaction was poured into H₂O and the organic phase removed. The aqueous phase was and extracted with CH₂Cl₂ (500 mL) and washed with brine (100 mL). The organic layers were combined, dried over Na₂SO₄ and concentrated under vacuum to give (S)-Boc-prolinal (IX-IVb) as yellow oil (45 g, yield 90%). ¹H NMR (300 MHz, DMSO-d₆) δ: 9.36 (m, 1H), 4.00 (m, 1H), 3.32 (m, 2H), 1.70-1.88 (m, 4H), 1.38 (m, 9H).

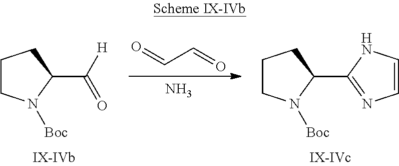

### General Procedure IX-M

Glyoxal (13 g, 0.23 mol) was added dropwise over 30 minutes to a solution of NH₄OH (200 mL) and Boc-prolinal (IX-IVb) (45 g, 2.3 mol) in THF. And the mixture was stirred at r.t. for 36 hours. The volatile component was removed in vacuo and the residue was purified by a flash chromatography (silica gel, ethyl acetate) followed by a recrystallization (ethyl acetate, room temperature) to provide compound IX-IVc as a white fluffy solid (7 g, yield 13%). ¹H NMR (300 MHz, CDCl₃) δ: 6.93 (s, 2H), 4.89-4.93 (m, 1H), 3.33-3.43 (m, 2H), 2.90-2.91 (m, 1H), 1.88-2.15 (m, 4H), 1.87 (s, 9H).

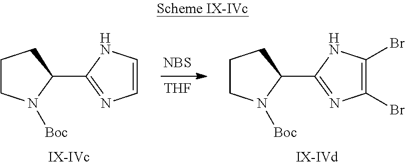

### General Procedure IX-N

To a solution of compound IX-IVc (6 g, 25 mmoL) in THF (100 mL) was added NBS (9 g, 51 mmoL) under 20° C. After additional, the mixture was warmed to room temperature and stirred overnight. The solution was concentrated and the residue was purified by chromatography on silica (PE:EA=10:1) to give the product to afford the compound IX-IVd (7 g, yield 70%). ¹H NMR (300 MHz, CDCl₃) δ4.82-4.88 (m, 1H), 3.36-3.38 (m, 2H), 2.78-2.83 (m, 1H), 1.93-2.01 (m, 3H), 1.43 (s, 9H).

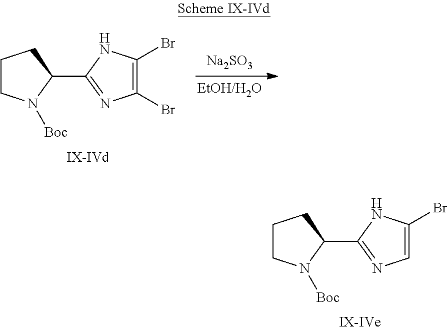

### General Procedure IX-O

To a suspension of compound IX-IVd (7 g, 18 mmoL) in EtOH/H₂O (90 mL, 1/2) was added Na₂SO₃ (22 g, 180 mmoL), the mixture was heated to reflux overnight. After filtered, the filtration was concentrated and purified by chromatography on silica (PE:EA=10:1) to afford compound IX-IVe (4 g, yield 67%). ¹H NMR (400 MHz, CDCl₃) δ: 10.58 (s, 1H), 6.85 (s, 1H), 4.80-4.83 (m, 1H), 3.28-3.32 (m, 2H), 2.81-2.84 (m, 1H), 1.86-2.05 (m, 3H), 1.32 (s, 9H).

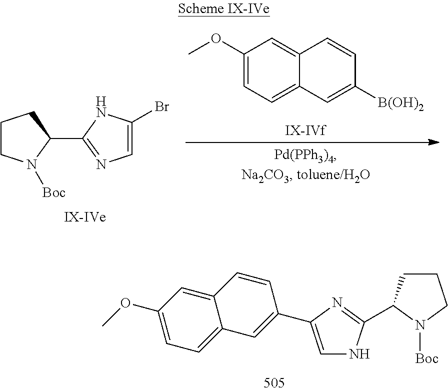

### General Procedure IX-P

To a solution of compound IX-IVe (100 mg 0.32 mmol), compound IX-IVf (95.89 mg 0.47 mmol) and Na₂CO₃ (67.1 mg, 0.63 mmol) in toluene (10 mL) and water (1 mL) was added Pd(PPh₃)₄ (4 mg, 0.03 mmol) under nitrogen in one portion at r.t. The resulting mixture was stirred at reflux overnight. Subsequently, TLC (PE:EA=2:1) indicated consumption of the starting material. Toluene was removed under reduced pressure and the residue was diluted with EtOAc (20 mL). The organic layer was washed with brine, dried and concentrated under reduced pressure. The residue was purified by column chromatograph on silica gel (eluting with PE:EA=4:1) to afford compound 505 (110 mg, yield 88%) as a white solid. ¹H NMR (300 MHz, CDCl₃) δ 12.0 (br, 1H), 8.09 (s, 1H), 7.81-7.67 (m, 3H), 7.48 (s, 1H), 7.19-7.17 (m, 1H), 7.06-7.03 (m, 1H), 4.72-4.61 (br, 1H), 3.79 (s, 3H), 3.32 (br, 2H), 2.12 (br, 1H), 1.98-1.81 (m, 3H), 1.2 (br, 9H). MS (ESI) m/z (M+H)⁺ 394.

### Example IX-V

### Preparation of Compound 506

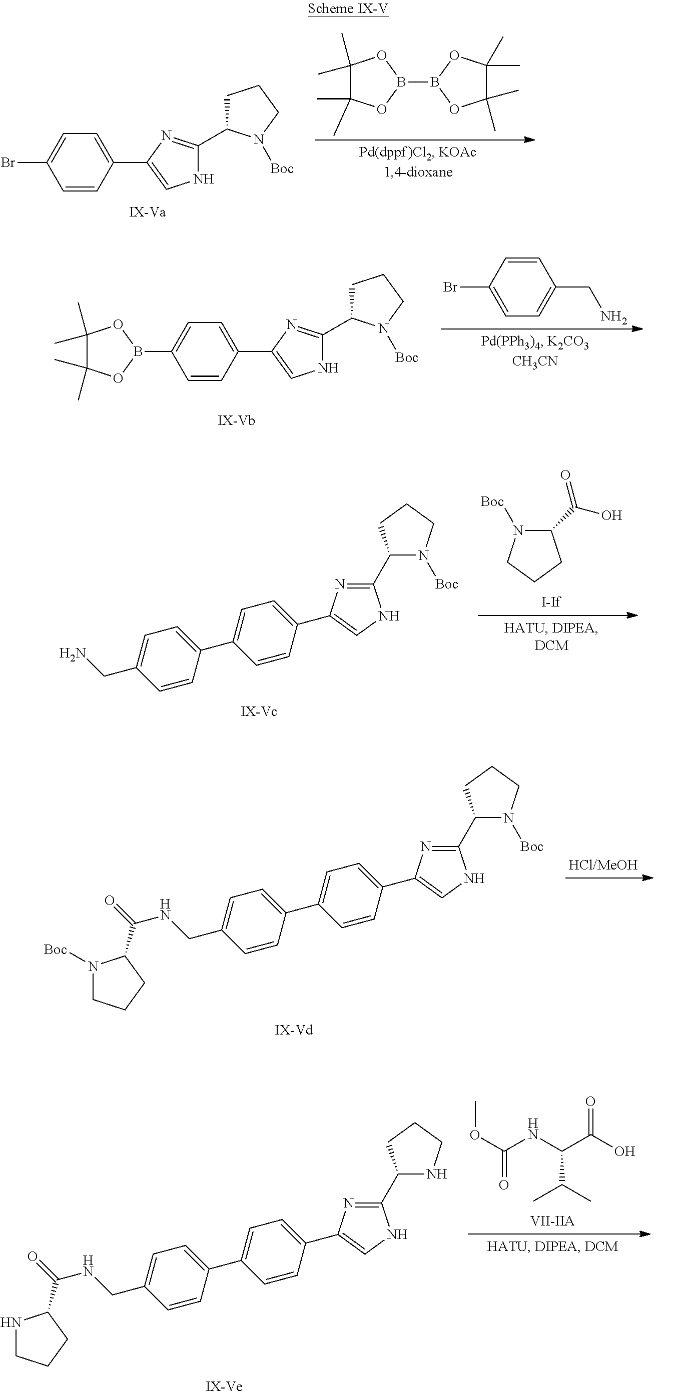

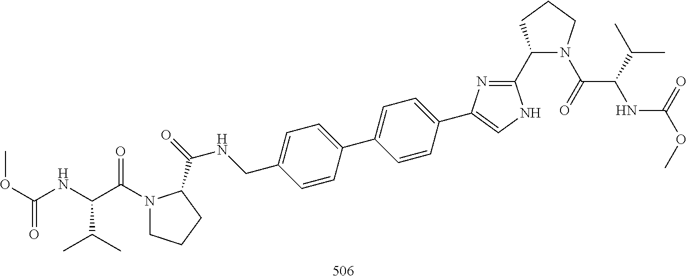

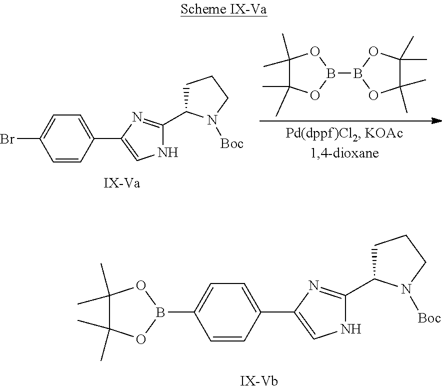

### General Procedure IX-Q

Pd(dppf)Cl₂ (0.2 g) was added into a mixture of compound IX-Va (2 g, 5.1 mmol). bis(pinacolato)diboron and KOAc (1.0 g, 10.2 mmol) in 1,4-dioxane (20 mL) under N₂, and the mixture was refluxed overnight. The mixture was cooled, filtered and concentrated, the crude product was purified by column chromatography on silica gel to provide compound IX-Vb (1.2 g, yield 53%).

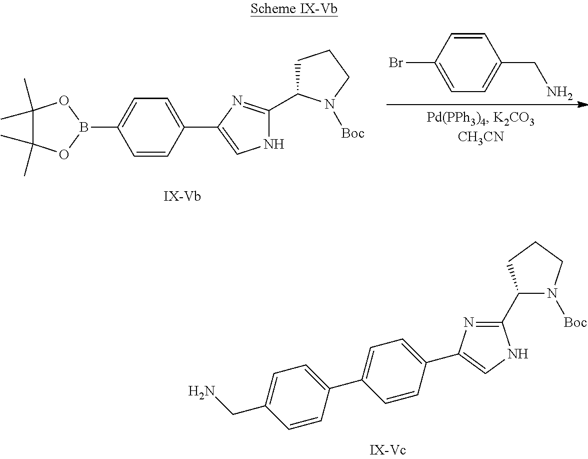

### General Procedure IX-R

Pd(PPh₃)₄ (0.03 g) was added into a mixture of 4-bromobenzylamine (0.14 g, 0.75 mmol), K₂CO₃ (0.19 g, 1.4 mmol) and compound IX-Vb (0.3 g, 0.68 mmol) in CH₃CN (3 mL) under N₂. Subsequently, the mixture was refluxed for 3 hours. The mixture was cooled to r.t., filtered and concentrated to give compound IX-Vc (0.3 g, yield 100%).

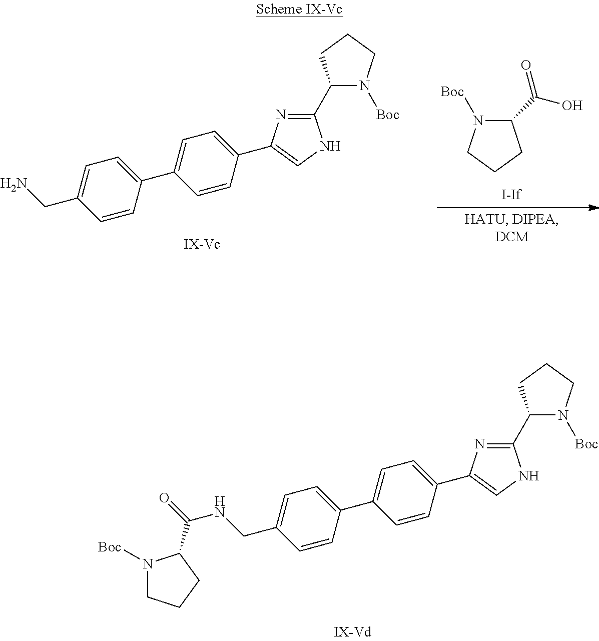

### General Procedure IX-S

N-Boc-proline (I-If; 0.22 g, 1.0 mmol) was dissolved in CH₂Cl₂ (5 mL) and treated with HATU (0.35 g, 0.9 mmol), DIEA (0.2 mL, 1.4 mmol) and stirred for 1 hour, then compound IX-Vc (0.3 g, 0.7 mmol) was added and the mixture was stirred at room temperature overnight. The mixture was filtered and concentrated in vacuo. The crude product was purified by column chromatography on silica gel to afford compound IX-Vd (0.25 g, yield 58%).

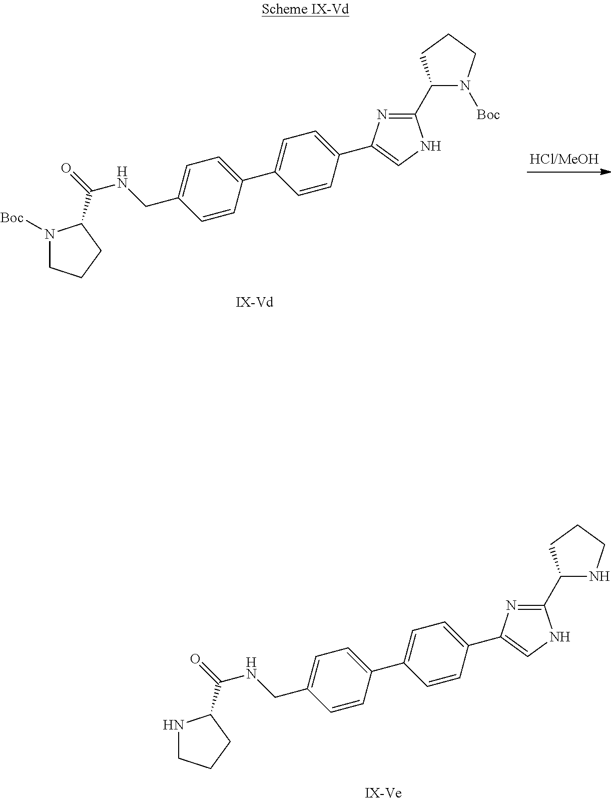

### General Procedure IX-T

Compound IX-Vd (0.25 g, 0.4 mmol) in a solution of HCl/CH₃OH (4 M, 2 mL) and stirred for 1 hour at r.t. And the mixture was concentrated under reduced pressure. The crude product IX-Ve was used directly in the next step without further purification.

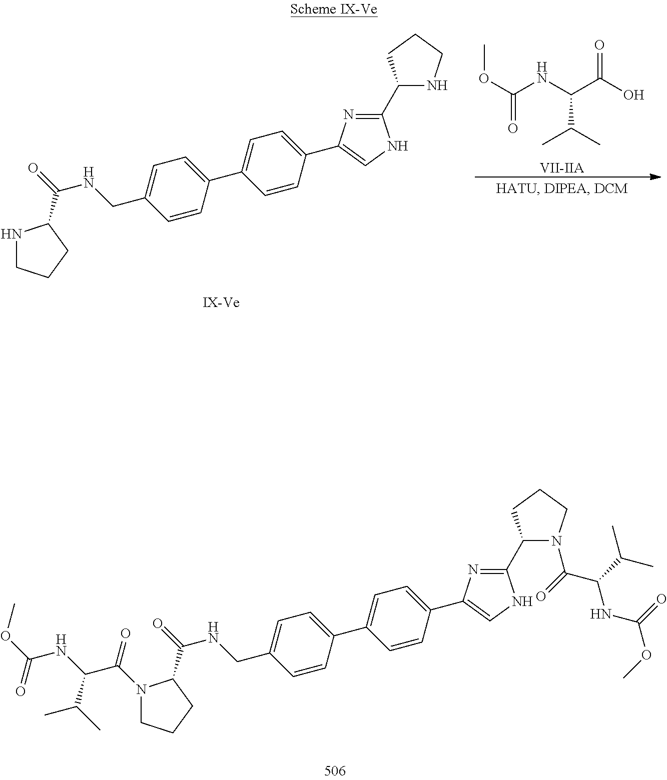

### General Procedure IX-U

Compound VII-IIA (0.18 g, 1.0 mmol) was dissolved in CH₂Cl₂ (25 mL) and treated with HATU (0.21 g, 0.63 mmol), DIEA (0.2 mL, 1.4 mmol) and stirred for 1 hour, then compound IX-Ve (0.2 g, 0.48 mmol) was added and the mixture was stirred at room temperature overnight. The mixture filtered and concentrated. The crude product was purified by silica gel chromatography to give compound 506 (0.085 g, yield 26%). MS (ESI) m/z (M+H)⁺ 730.2.

### Section X

### Preparation of Compounds: Section X

### Example X-I

### Preparation of Compound 601

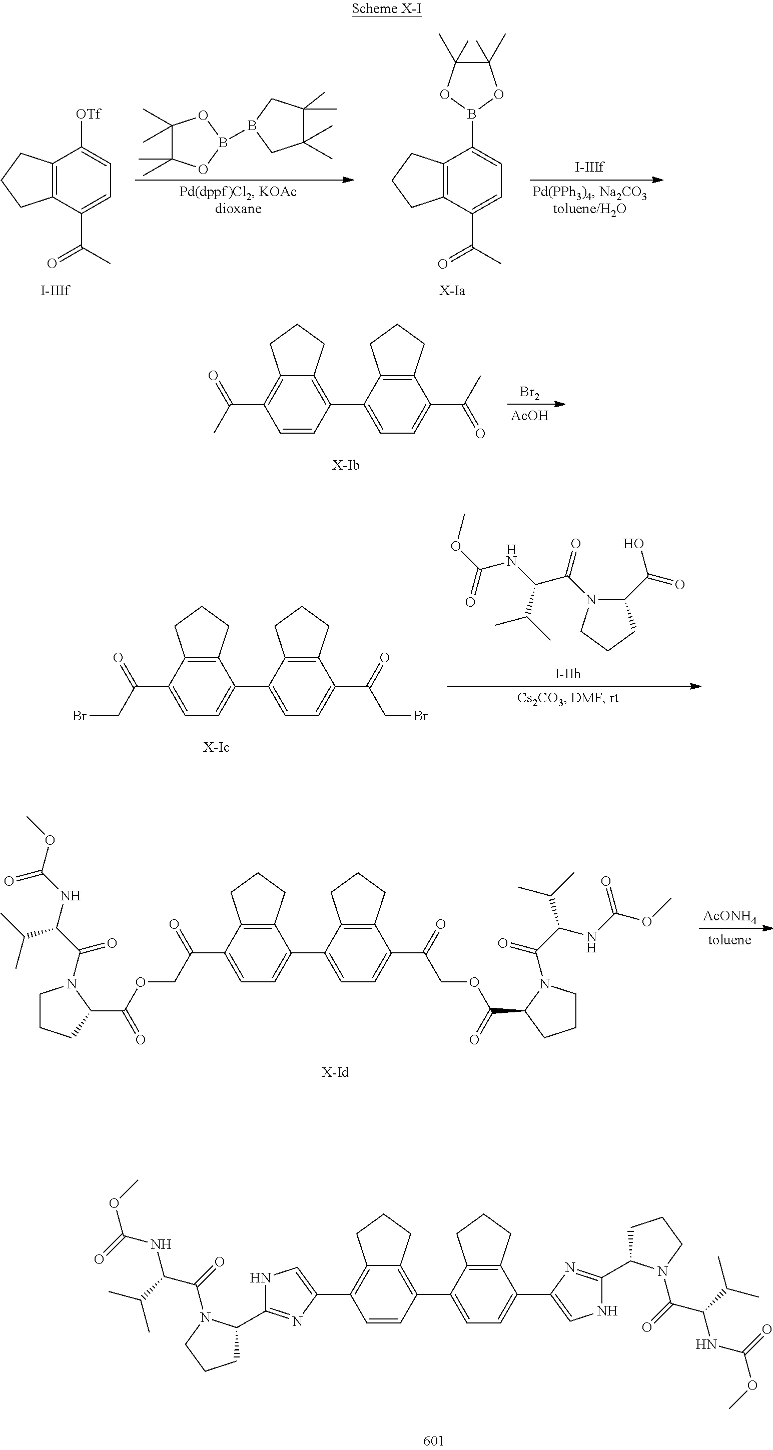

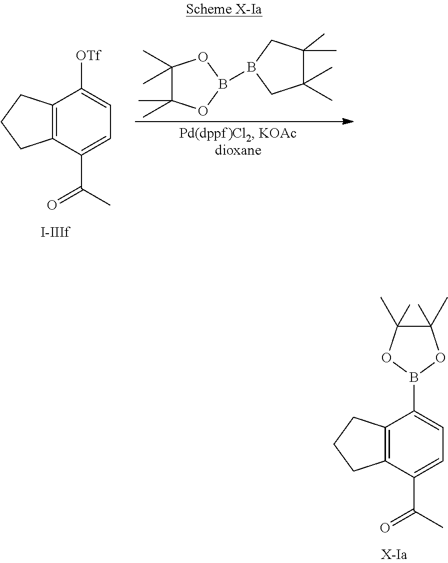

### General Procedure X-A

To a solution of compound I-IIIf (0.6 g, 1.95 mmol) in dioxane (10 mL) was added bis(pinacolato)diboron (0.54 g, 2.14 mmol), KOAc (0.38 g, 3.90 mmol) and Pd(dppf)Cl₂ (0.05 g, catalyzed amount). The mixture was purged with nitrogen for 5 minutes and heated to 90° C. overnight. After being cooled to room temperature, the mixture was diluted with water (20 mL) and extracted with EtOAc (50 mL×3). The combined organic layers were washed with brine (20 mL×2), filtered, the filtrate was concentrated under reduced pressure. The residue was purified by Prep-TLC (eluted with PE:EtOAc=20:1) to give compound X-Ia (0.4 g, yield: 73%) as an-off yellow solid. MS (ESI) m/z (M+H)⁺ 287.0. ¹H NMR (300 MHz, CDCl₃) δ 7.65 (m, 2H), 3.21 (t, 2H), 3.12 (t, 2H), 2.60 (s, 3 H), 2.03 (m, 2H), 1.35 (s, 12H).

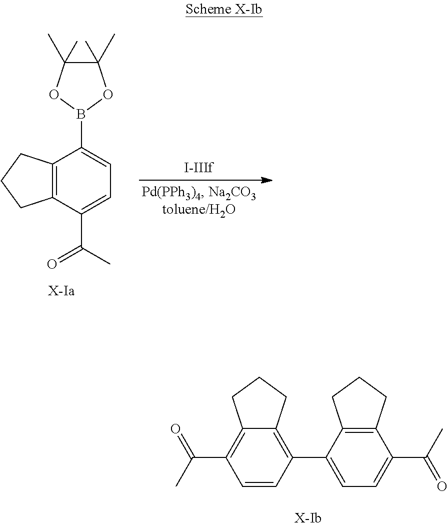

### General Procedure X-B

To a solution of compound X-Ia (0.37 g, 1.30 mmol) in toluene/H₂O (10 mL/1 mL), Na₂CO₃ (0.28 g, 2.6 mmol) and compound I-IIIf (0.40 g, 1.30 mmol) were added, the resulting mixture was purged with nitrogen, then Pd(PPh₃)₄ (10 mg, catalyzed amount) was added. The reaction mixture was stirred at 80° C. overnight under nitrogen protection. TLC monitored the reaction. After completion of the reaction, the mixture was poured into water (50 mL), extracted with EtOAc (50 mL×3), the combined organic layers were dried over Na₂SO₄, concentrated in vacuo. The residue was purified by Prep-TLC (eluted with PE:EtOAc=20:1) to afford compound X-Ib as a yellow oil (0.2 g, yield: 48%). ¹H NMR (300 MHz, CDCl₃) δ 7.73 (d, J=10.4 Hz, 2H), 7.14 (d, J=10.4 Hz, 2H), 3.33 (t, 2H), 2.67 (t, 2H), 2.63 (s, 6H), 2.09-1.99 (m, 6H).

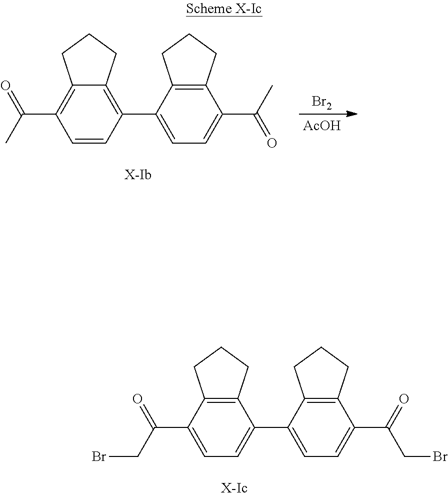

### General Procedure X-C

To a mixture of compound X-Ib (0.20 g, 0.63 mmol) in HOAc (9 mL) was added a solution of Br₂ (0.2 g, 1.26 mmol) in HOAc (1 mL) dropwise and the resulting mixture was stirred at 30° C. overnight. EtOAc (50 mL) was added into the mixture and washed with aq. sat. NaHCO₃ (3×50 mL). The organic layer was concentrated under reduced pressure to give compound X-Ic, which was used directly in the next step. MS (ESI) m/z (M+H)⁺ 287.0.

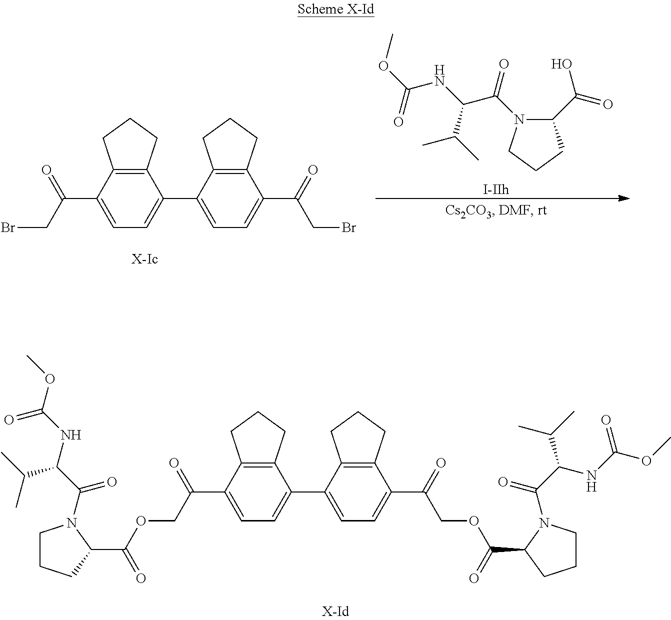

### General Procedure X-D

Cs₂CO₃ (0.27 g, 0.84 mmol) and compound I-IIh (0.23 g, 0.84 mmol) were added to a suspension of compound X-Ic (0.1 g, 0.21 mmol) in DMF (5 mL). The resulting mixture was stirred at rt for 3 hours. The reaction mixture was diluted with water (20 mL) and extracted with EtOAc (50 mL×3). The combined organic layers were washed with water (5 mL×5), brine (5 mL×2), filtered, the filtrate was concentrated under reduced pressure. The crude product was purified by Prep-TLC (eluted with petroleum ether:EtOAc=1:1) to give compound X-Id as a yellow solid (0.08 g, the total yield of two step: 15%). MS (ESI) m/z (M+H)⁺ 476.8.

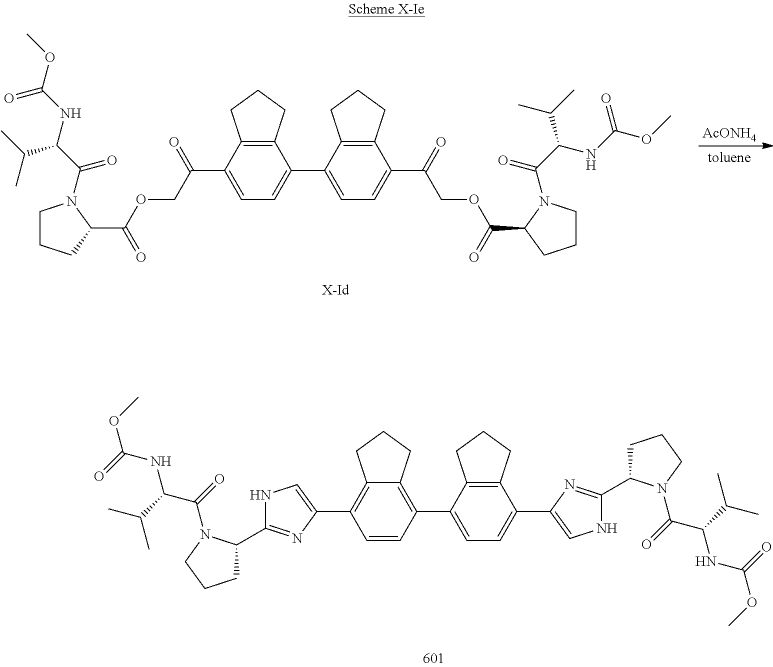

### General Procedure X-E

A solution of compound X-Id (0.08 g, 0.093 mmol) in dry toluene (5 mL) was added ammonium acetate (0.072 g, 0.93 mmol). The mixture was stirred with refluxing overnight. The mixture was diluted with water (20 mL) and extracted with EtOAc (20 mL×3). The combined organic layers were dried over Na₂SO₄, concentrated in vacuo. The residue was purified by Prep-TLC to afford compound 601 as a yellow solid (0.05 g, yield: 66%). ¹H NMR (400 MHz, CDCl₃) δ 7.13 (m, 6H), 5.45 (d, J=8.8 Hz, 2H), 5.28 (d, J=5.6 Hz, 2H), 4.33 (t, 2H), 4.82 (m, 2H), 3.70-3.63 (m, 10H), 3.12-3.09 (m, 6H), 2.79-2.78 (m, 4H), 2.35 (m, 2H), 2.23-1.96 (m, 6H), 0.88 (d, J=3.6 Hz, 12H). MS (ESI) m/z (M+H)⁺ 819.3.

### Example X-II

### Preparation of Compound 602

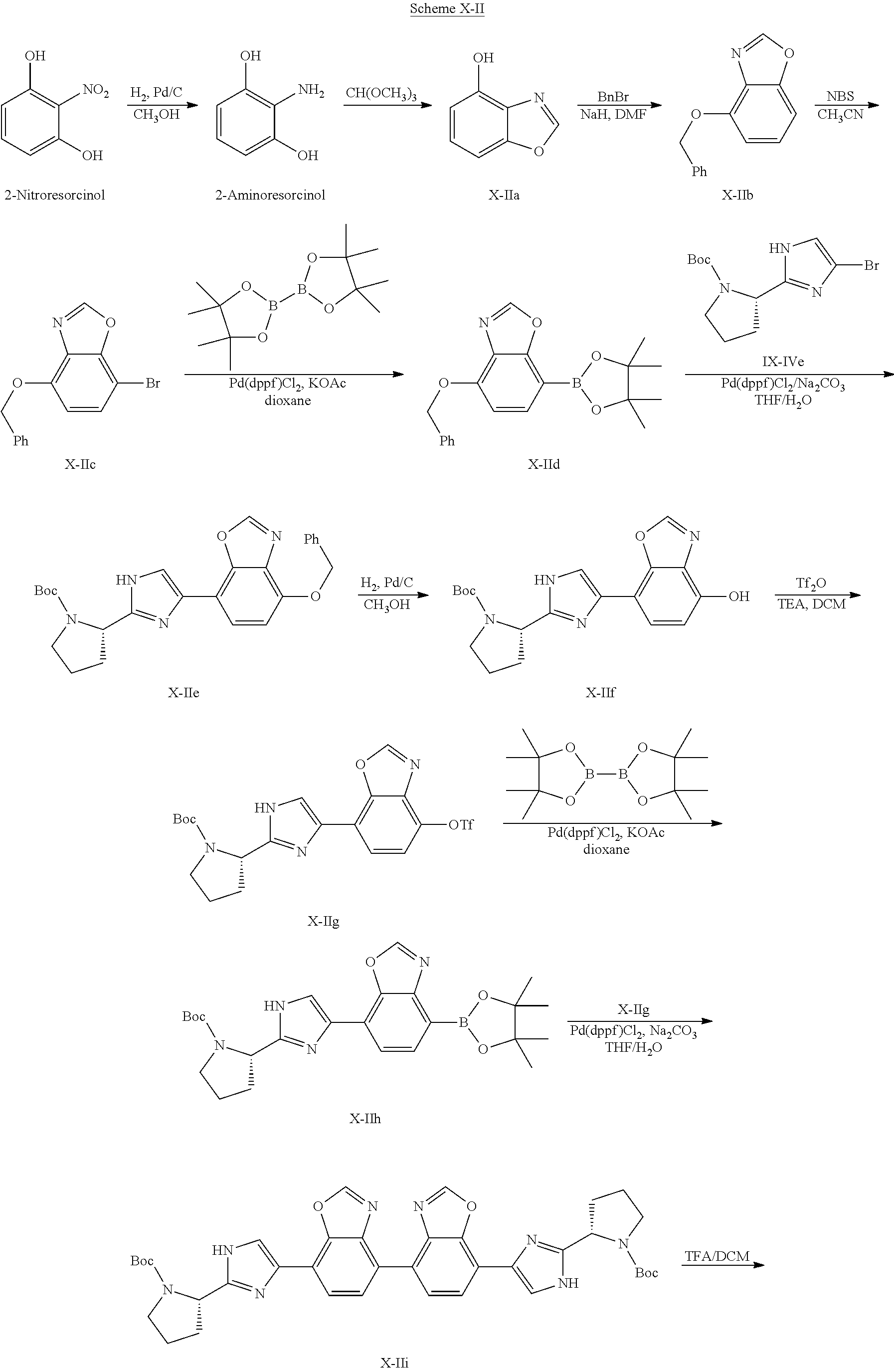

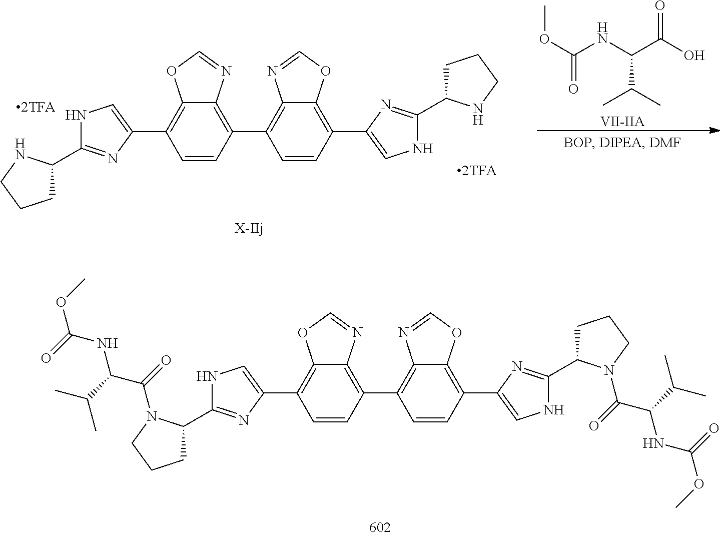

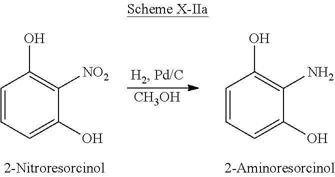

### General Procedure X-F

Pd/C (10 g, 10%) was added to the mixture of 2-nitroresorcinol (89.0 g, 0.57 mmol) in 1 L of methanol. The mixture was placed under hydrogen using a hydrogen balloon and hydrogenated at room temperature for 4 hours. After removal of the hydrogen, the catalyst was filtered off through celite, the filtrate was concentrated to give crude 2-aminoresorcinol (75.0 g, yield 99%).

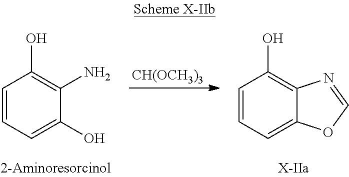

### General Procedure X-G

A mixture of 2-aminoresorcinol (31 g, 0.25 mol), and CH(OCH₃)₃ (56.00 g, 0.38 mol) was stirred at 130° C. for 2 hours under nitrogen protection. TLC monitored the reaction. After completion of the reaction, the mixture was concentrated in vacuo. The residue was purified by column chromatography on silica gel (PE:EtOAc=2:1) to afford compound X-IIa (24 g, yield 72%). MS (ESI) m/e (M+H)⁺:136.

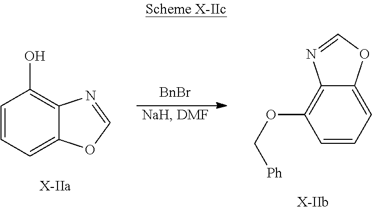

### General Procedure X-H

To a stirred solution of compound X-IIa (20 g, 0.14 mol) in DMF was added NaH (60% dispersion in mineral oil, 6.52 g, 0.16 mmol) at 0° C. under argon. The solution was stirred for 0.5 hour. Then BnBr (29.00 g, 0.16 mol) was added dropwise at 0° C. under argon, and the mixture was warmed slowly to room temperature, and stirred for additional 3 hours. Subsequently, H₂O (60 mL) was added, and the mixture was extracted with EtOAc (60 mL×3). The organic layer was separated, washed with the brine, dried over Na₂SO₄ and concentrated in vacuo. The residue was purified by column chromatography on silica gel (PE:EtOAc=5:1) to afford compound X-IIb (31 g, yield 94%).

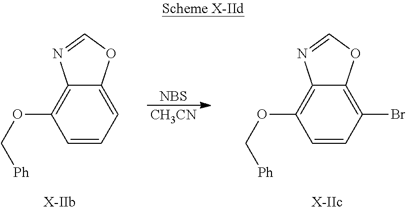

### General Procedure X-I

To a solution of compound X-IIb (21.00 g, 0.13 mmol) in CH₃CN (1000 mL) was added NBS (26.00 g, 0.14 mmol) under nitrogen protection. The reaction mixture was then stirred at the room temperature overnight. After completion of the reaction, the mixture was concentrated in vacuo. The residue was purified by Prep-HPLC to to afford compound X-IIc (27 g, yield 65%). MS (ESI) m/e (M+H)⁺:305.

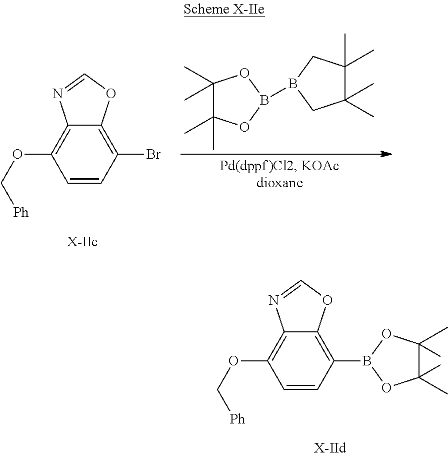

### General Procedure X-J

The mixture of compound X-IIc (6.00 g, 19.67 mmol), Bis(pinacolato)diboron (9.99 g, 39.33 mmol), KOAc (3.86 g, 39.38 mmol) and Pd(dppf)Cl₂ (719 mg, 0.98 mmol) in 100 mL of 1,4-dioxane was stirred at reflux under argon for 4 hours. After concentration, the residue was partitioned between H₂O and DCM, the aqueous phase was extracted with DCM, and the combined organic layer was washed with brine, dried over Na₂SO₄, and concentrated. The residue was purified by column chromatography on silica gel to afford compound X-IId (5.5 g, yield: 89%).

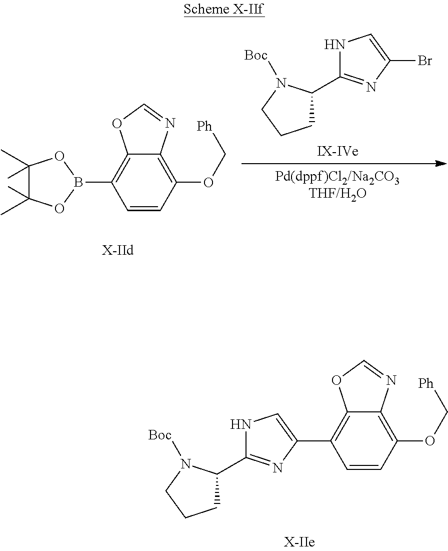

### General Procedure X-K

A mixture of compound X-IId (2.50 g, 7.12 mmol), compound IX-IVe (2.24 g, 7.08 mmol), Na₂CO₃ (1.51 mg, 14.24 mmol) and Pd(dppf)Cl₂ (363 mg, 0.49 mmol) in 50 mL of THF/H₂O (v/v=5:1) was refluxed overnight under argon. After concentration, the residue was partitioned between H₂O and DCM, the aqueous phase was extracted with DCM. The combined organic layer was washed with brine, dried over Na₂SO₄ and concentrated. The residue was purified by column chromatography on silica gel (PE:EtOAc=1:1) to afford compound X-IIe (2.3 g, yield 66%). MS (ESI) m/z (M+H)⁺ 461.

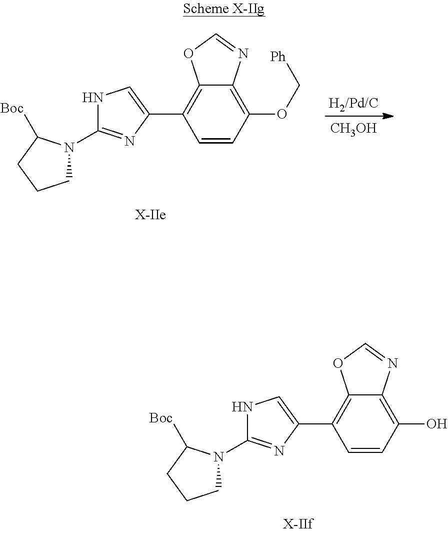

### General Procedure X-L

To a mixture of compound X-IIe (1.20 g, 2.60 mmol) in 20 mL of methanol was added Pd/C (120 mg, 10%). The mixture was placed under hydrogen using a hydrogen balloon and hydrogenated at room temperature overnight. After the completion of the reaction and removal of the hydrogen, the catalyst was filtered off through celite, and the filtrate was concentrated to give the crude product X-IIf (0.80 g, yield 86%). MS (ESI) m/z (M+H)⁺ 371.

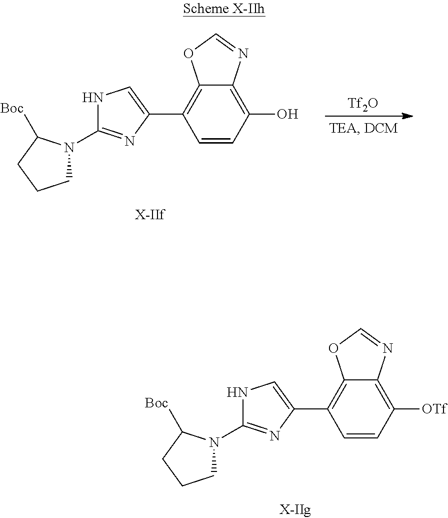

### General Procedure X-M

To a stirred solution of compound X-IIf (0.8 mg, 2.16 mmol) and Et₃N (328 mg) in DCM was added dropwise Tf₂O (334 mg, 1.18 mmol) at −78° C. under argon. After addition, the solution was stirred for 0.5 hour, then warmed slowly to the room temperature, and stirred for additional 3 hours. Then H₂O (5 mL) was added and extracted with EtOAc (10 mL×3). The organic layer was separated, washed with the brine, dried over Na₂SO₄ and concentrated under reduced pressure. The residue was purified by column chromatography on silica gel to afford compound X-IIg (240 mg, yield 52%). MS (ESI) m/z (M+H)⁺ 503.

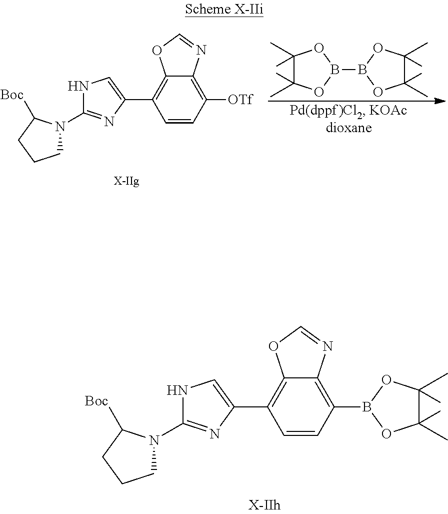

### General Procedure X-N

The mixture of compound X-IIg (287 mg, 0.57 mmol), bis(pinacolato)diboron (290 mg, 1.14 mmol), KOAc (112 mg, 1.14 mmol) and Pd(dppf)Cl₂ (21 mg, 0.03 mmol) in 10 mL of 1,4-dioxane was refluxed under argon for 4 hours. After concentration, the residue was partitioned between H₂O and DCM. The organic layer was separated, washed with brine, dried over Na₂SO₄, concentrated under reduced pressure. The residue was purified by column chromatography on silica gel to yield compound X-IIh (240 mg, yield 78%). MS (ESI) m/z (M+H)⁺ 481.

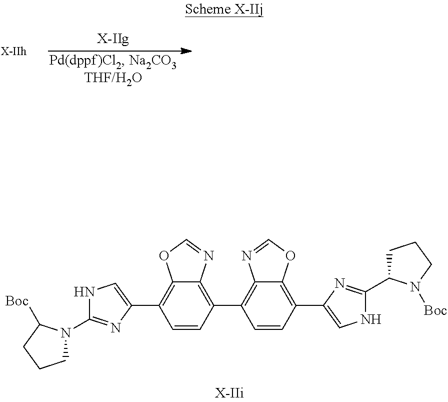

### General Procedure X-O

The mixture of compound X-IIh (120 mg, 0.23 mmol), compound X-IIg (115 mg, 0.23 mmol), Na₂CO₃ (51 mg, 0.06 mmol) and Pd(dppf)Cl₂ (9 mg, 0.01 mmol) in 6 mL of THF:H₂O (v:v=5:1) was refluxed overnight under argon. After concentration, the residue was partitioned between H₂O and DCM, the organic layer was separated, washed with brine, dried over Na₂SO₄, and concentrated under reduced pressure. The residue was purified by Prep-HPLC to provide compound X-IIi (40 mg, yield 21%). MS (ESI) m/z (M+H)⁺ 707.

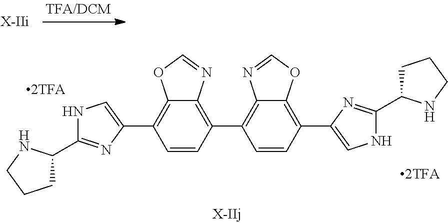

### General Procedure X-P

Compound X-IIi (30 mg, 0.04 mmol) was added to a solution of TFA/DCM (10 mL, 1/1). And the reaction mixture was stirred at room temperature for 2 hrs. When the reaction completed, the mixture was concentrated under vacuum to afford compound X-IIj (30 mg, yield 80%). MS (ESI) m/e (M+H)⁺: 507.

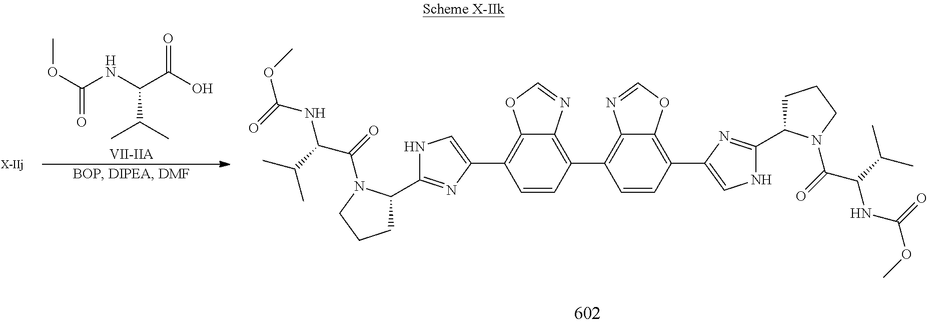

### General Procedure X-Q

To a mixture of compound X-IIj (53 mg, 0.10 mmol), compound VII-IIA (37 mg, 0.21 mmol) and DIPEA (81 mg, 0.63 mmol) in DMF (3 mL) was added BOP (92 mg, 0.21 mmol). The resulting mixture was stirred overnight at room temperature. After concentration under reduced pressure, the residue was purified by Prep-HPLC to afford compound 602 (6.70 mg, yield 10%). ¹H NMR (400 MHz, CD₃OD): δ 8.61 (s, 2H), 8.18-8.04 (m, 4H), 7.82 (s, 2H), 5.28-5.25 (m, 2H), 4.30-4.26 (m, 2H), 4.04-4.01 (m, 2H), 3.95-3.92 (m, 2H), 3.68 (s, 6H), 2.42-2.25 (m, 5H), 2.13-2.06 (m, 5H), 1.03-0.87 (m, 12H). MS (ESI) m/e (M+H)⁺: 821.4.

### Example X-III

### Preparation of Compound 603

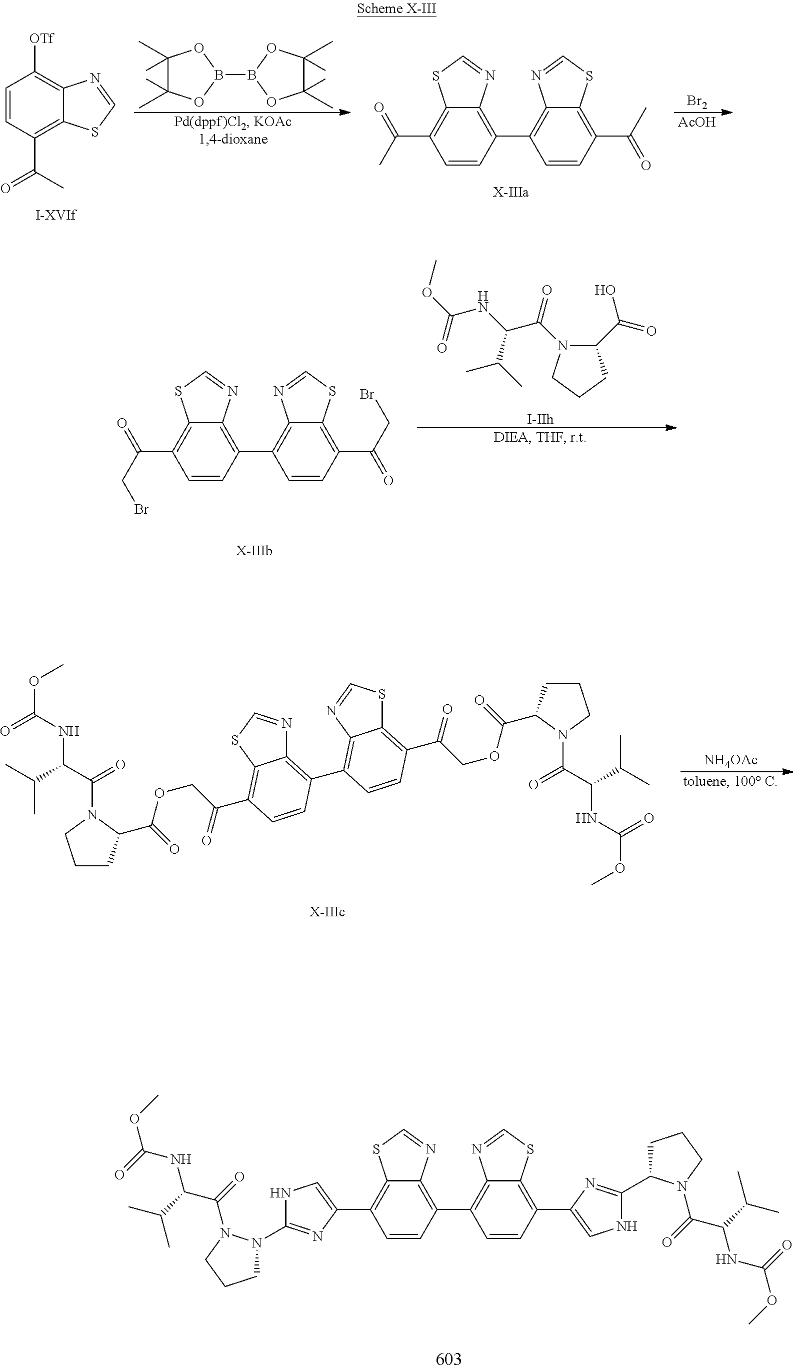

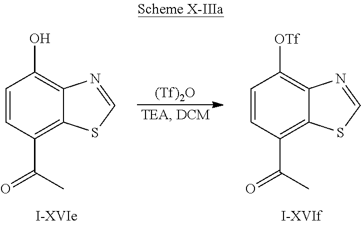

### General Procedure X-R

Tf₂O (0.19 mL, 1.14 mmol) was added to a stirred solution of compound I-XVIe (200 mg, 1.04 mmol) and TEA (0.2 mL, 1.55 mmol) in DCM (10 mL) at 0° C. The reaction mixture was stirred for 30 min, then poured into ice-water, and extracted with DCM (50 mL×3). The combined organic layers were dried over MgSO₄ and concentrated in vacuo to afford compound I-XVIf (0.34 g, crude yield 100%).

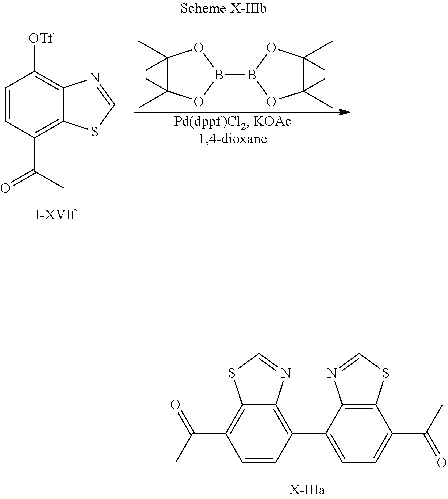

### General Procedure X-S

To a stirred mixture of compound I-XVIf (340 mg, 1.02 mmol), bis(pinacolato)diboron (387 mg, 1.52 mmol) and KOAc (203 mg, 2.04 mmol) in 1,4-dioxane (10 ml) was added Pd(dppf)Cl₂ (85 mg) under N₂ protection. The mixture was stirred at 90° C. for 3 hrs. After the reaction completed, the mixture was cooled down to r.t. and diluted with DCM (100 mL), washed with water and brine, the organic layers was dried over MgSO₄, filtered and concentrated. The residue was recrystallized from MeOH to afford compound X-IIIa (130 mg, crude yield 73%). MS (ESI) m/z (M+H)⁺ 353.1. ¹H NMR (400 MHz, CDCl₃) δ 9.19 (s, 2H), 8.28 (d, J=8 Hz, 2H), 8.06 (d, J=8 Hz, 2H), 2.85 (s, 6H).

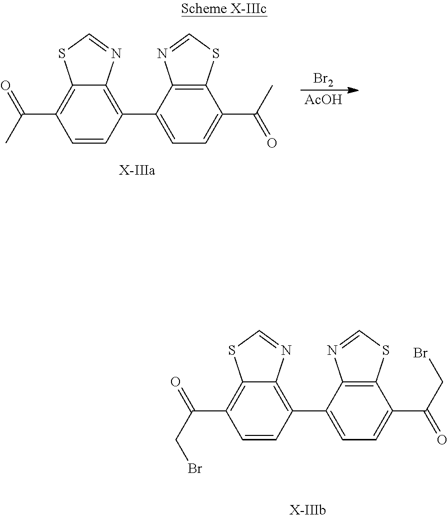

### General Procedure X-T

Bromine (Br₂, 193 mg, 1.2 mmol) was added to a stirred solution of compound X-IIIa (170 mg, 0.48 mmol) and in AcOH (3 mL) at 90° C. The reaction mixture was stirred for 3 hours, then poured into ice-water, neutralized with saturated aq. NaHCO₃ and extracted with DCM (50 mL×3). The combined organic layers were dried over MgSO₄ and concentrated in vacuo to afford the crude compound X-IIIb (0.15 g, crude yield 62%).

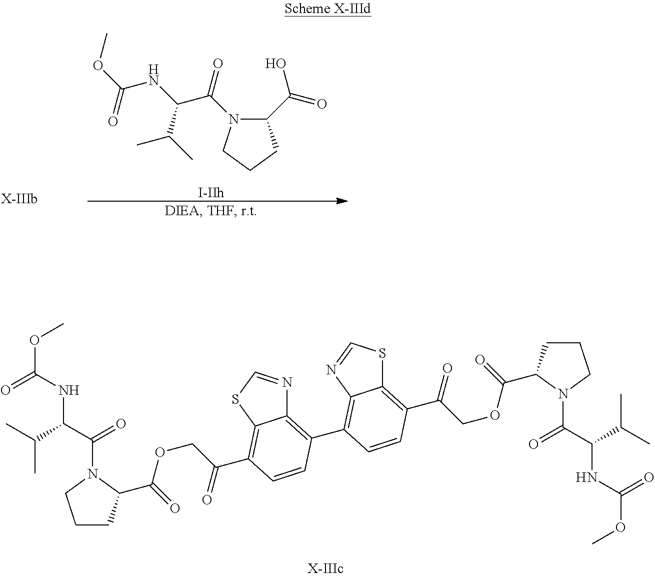

### General Procedure X-U

To a stirred mixture of compound X-IIIb (150 mg, 0.29 mmol), DIEA (0.5 mL) in THF (5 mL) was added compound I-IIh (160 mg, 0.59 mmol). The mixture was stirred at r.t for 4 hrs. Then the mixture was diluted with EtOAc (100 mL), washed with water and brine, the organic layers was dried over MgSO₄, filtered and concentrated. The residue was purified by prep-TLC to afford compound X-IIIc (25 mg, yield 9%). MS (ESI) m/z (M+H)⁺ 893.

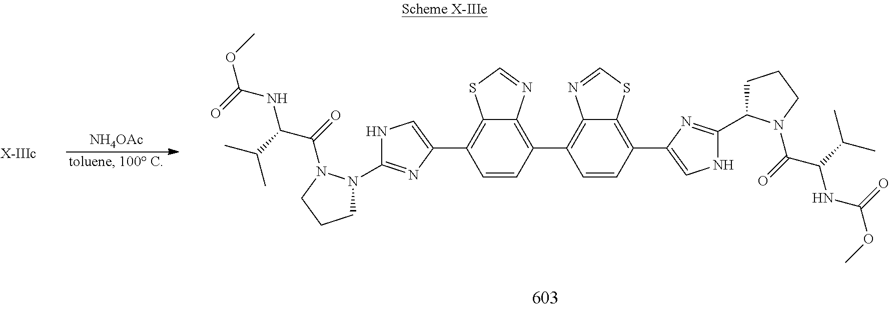

### General Procedure X-V

A mixture of compound X-IIIc (25 mg, 0.028 mmol) and NH₄OAc (21 mg, 0.28 mmol) in toluene (5 mL) was stirred overnight at 100° C. After the reaction completed, the mixture was cooled down to r.t. and diluted with EtOAc (60 mL), washed with water and brine, the organic layers were dried over MgSO₄, filtered and concentrated. The residue was purified by prep-HPLC to afford 603 (5.2 mg, yield 22%). MS (ESI) m/z (M+H/2)⁺ 853.2.

### Example X-IV

### Preparation of Compound 604

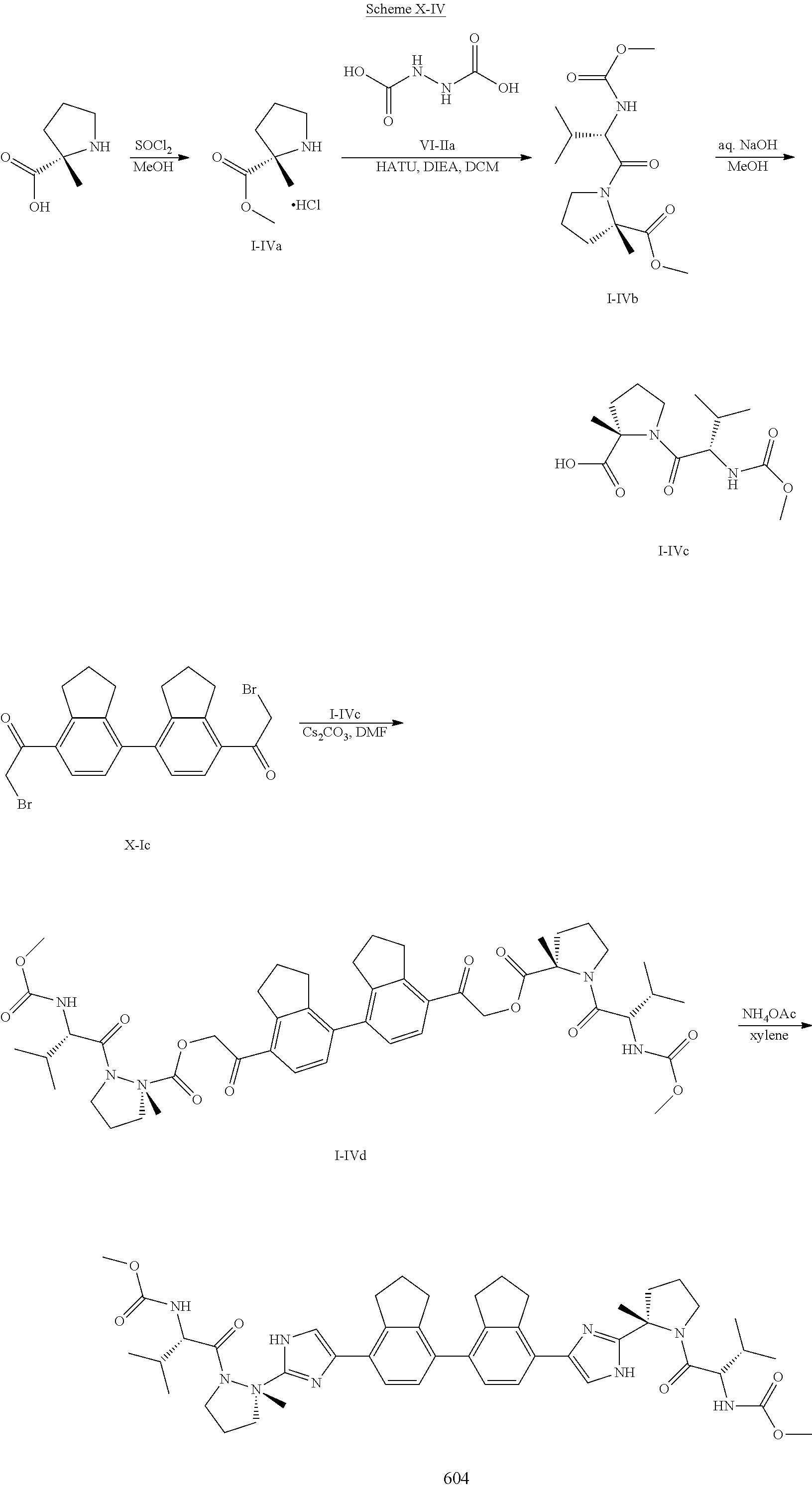

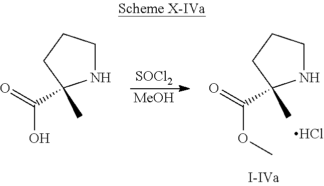

### General Procedure X-W

To a mixture of 2-methyl-L-proline (1.0 g, 7.8 mmol) in 20 mL of dry methanol was added SOCl₂ (2.8 g, 23.3 mmol) dropwise at 0° C. under nitrogen protection. The resulting mixture was stirred at room temperature overnight, and then the solvent was removed under reduced pressure to afford compound I-IVa as an HCl salt (1.4 g, yield 100%). ¹H NMR (300 MHz, CD₃OD): δ 3.86 (s, 3H), 3.42-3.46 (m, 2H), 2.36-2.45 (m, 1H), 2.00-2.19 (m, 3H), 1.68 (s, 3H).

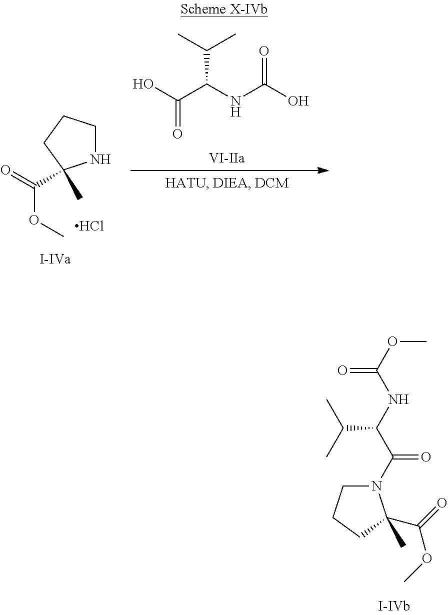

### General Procedure X-X

To a solution of compound I-IVa (1.35 g, 7.7 mmol) in 30 mL of DCM was added compound VI-IIa (1.5 g, 8.5 mmol), HATU (4.4 g, 11.6 mmol) and DIEA (3 g, 23 mmol). The resulting mixture was stirred at room temperature overnight. Subsequently, the mixture was diluted with DCM and washed with brine. The organic layers were dried over anhydrous Na₂SO₄ and concentrated. The residue was purified by column chromatography (PE/EA=3/1) to afford compound I-IVb (1.5 g, yield 65%). MS (ESI) m/z (M+H)⁺ 301.

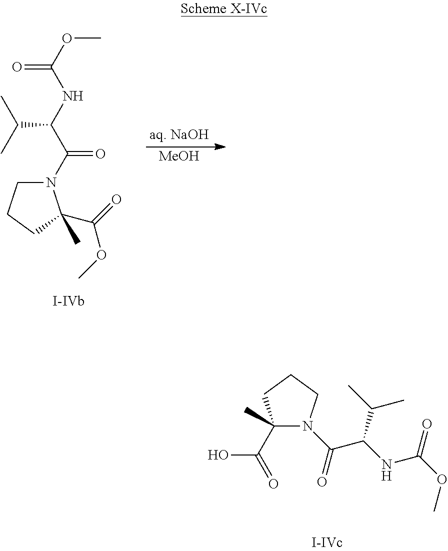

### General Procedure X-Y

A mixture of compound I-IVb (1.5 g, 5 mmol) and NaOH (0.6 g, 15 mmol) in MeOH (30 mL) and H₂O (5 mL) was stirred at 70° C. for 2 hours. The methanol under reduce pressure and the residue was dissolved with 20 mL of H₂O, then the solution was acidified to pH 2˜3 with 2N HCl and extracted with DCM (50 mL×2). The organic layer was washed with brine, dried over anhydrous Na₂SO₄ and concentrated to afford compound I-IVc (0.8 g, yield 57%), which was used in next step without further purification. ¹H NMR (300 MHz, DMSO-d₆): δ 12.20 (s, 1H), 7.24 (d, J=8.4 Hz, 1H), 3.54-3.98 (m, 3H), 3.50 (s, 3H), 1.78-2.05 (m, 5H), 1.34 (s, 3H), 0.84-0.88 (m, 6H).

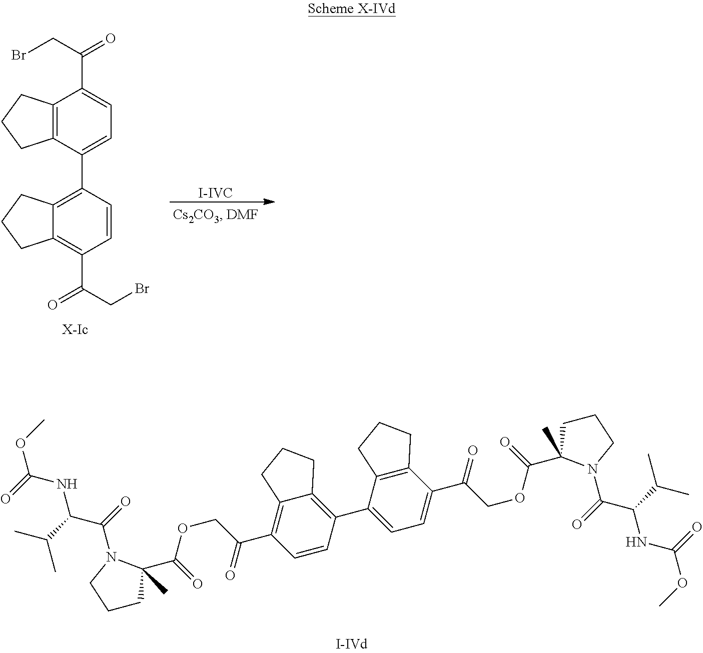

### General Procedure X-Z

A mixture of compound X-Ic (100 mg, 0.21 mmol), compound I-IVc (150 mg, 0.53 mmol) and Cs₂CO₃ (137 mg, 0.42 mmol) in 5 mL of DMF was stirred at room temperature for 2 hours. Then the mixture was diluted with EtOAc (30 mL), and washed with brine (30 mL). The organic layer was separated, dried over anhydrous Na₂SO₄ and concentrated. The residue was purified by Prep-TLC (DCM/MeOH=20/1) to afford compound I-IVd (140 mg, yield 75%). MS (ESI) m/z (M+H)⁺887.

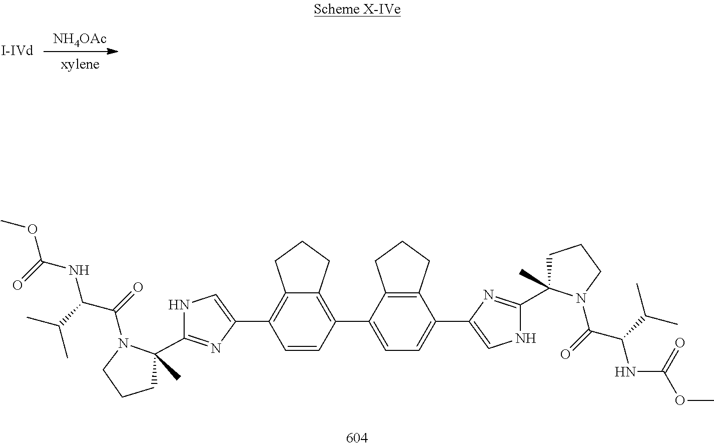

### General Procedure X-AA

A mixture of compound I-IVd (140 mg, 0.16 mmol) and NH₄OAc (243 mg, 3.2 mmol) in 10 mL of xylene was stirred at 120° C. for 5 hours in a sealed tube. After cooling to room temperature, the solvent was removed under reduce pressure and the residue was diluted with EtOAc (40 mL), and washed with brine (30 mL). The organic layer was separated, dried over anhydrous Na₂SO₄ and concentrated. The residue was purified by Prep-HPLC to afford compound 604 (35 mg, yield 26%). ¹H NMR (300 MHz, MeOD): δ 7.49-7.54 (m, 2H), 7.05-7.10 (m, 4H), 4.18 (d, J=7.8 Hz, 2H), 4.00-4.06 (m, 2H), 3.86-3.94 (m, 2H), 3.65 (s, 6H), 3.05-3.10 (m, 4H), 2.73-2.80 (m, 4H), 2.54-2.63 (m, 2H), 2.01-2.15 (m, 12H), 1.87 (s, 6H), 0.87-0.96 (m, 12H). MS (ESI) m/z (M+H)⁺ 847.6.

### Example X-V

### Preparation of Compound 605

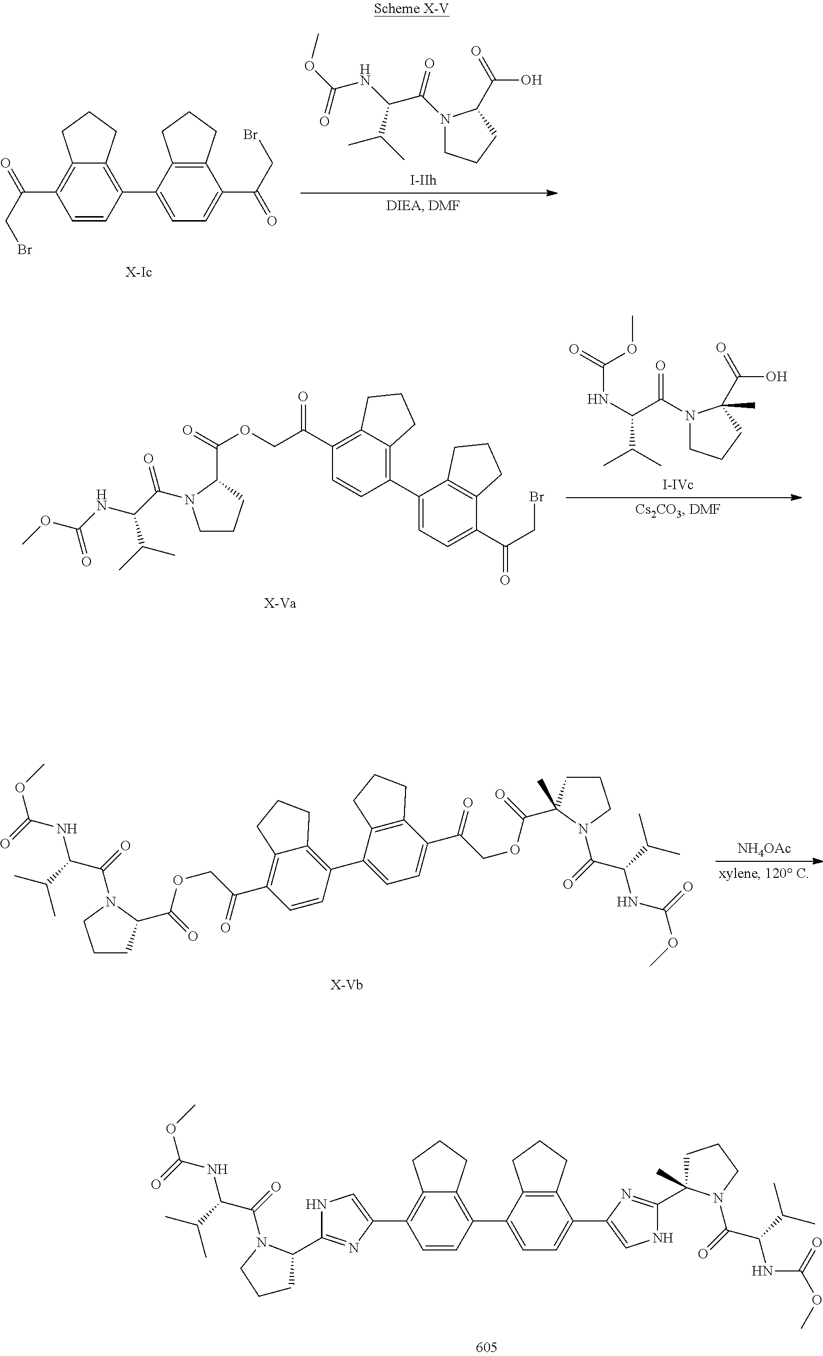

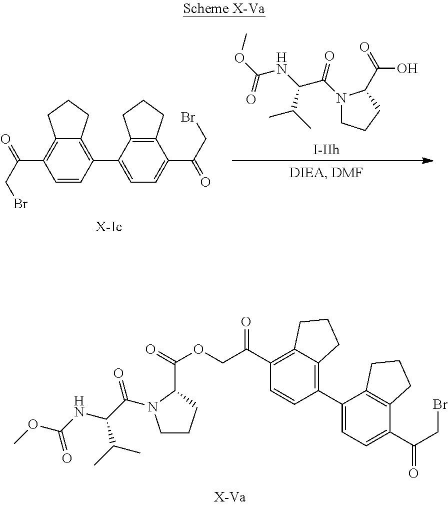

### General Procedure X-AB

A mixture of compound X-Ic (100 mg, 0.21 mmol), compound I-IIh (57 mg, 0.21 mmol), DIEA (82 mg, 0.63 mmol) in DMF (5 mL) was stirred at room temperature for 2 hrs. The mixture was diluted with EtOAc (30 mL), and then washed with water and brine. The organic layers were dried over anhydrous Na₂SO₄ and concentrated under reduced pressure to give a crude residue. The crude residue was purified by prep-TLC (Petroleum ether:EtOAc=1:1) to afford compound X-Va (40 mg, yield 29%). MS (ESI) m/z (M+H)⁺ 668.

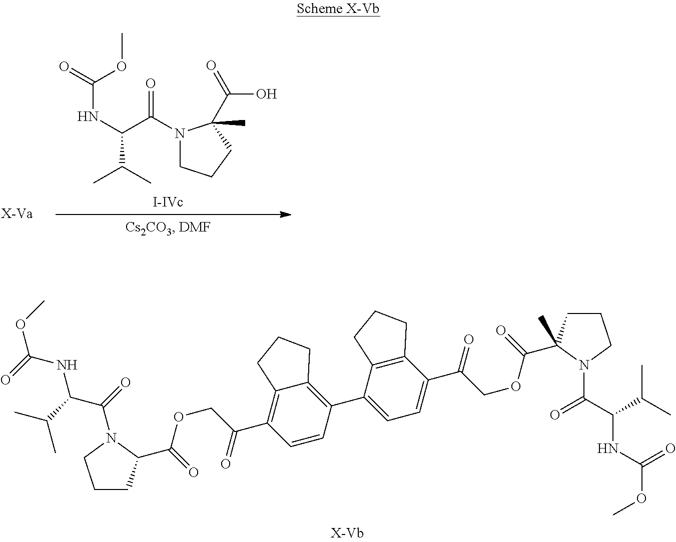

### General Procedure X-AC

A mixture of compound X-Va (60 mg, 0.09 mmol), compound I-IVc (26 mg, 0.09 mmol) and Cs₂CO₃ (58 mg, 0.18 mmol) in DMF (3 mL) was stirred at room temperature for 2 hrs. The mixture was diluted with EtOAc (30 mL), and washed with water and brine. The organic layers were separated, dried over anhydrous Na₂SO₄ and concentrated under reduced pressure to give a crude residue. The residue was purified by prep-TLC (DCM/MeOH=20/1) to afford compound X-Vb (30 mg, yield 38%). MS (ESI) m/z (M+H)⁺ 874.

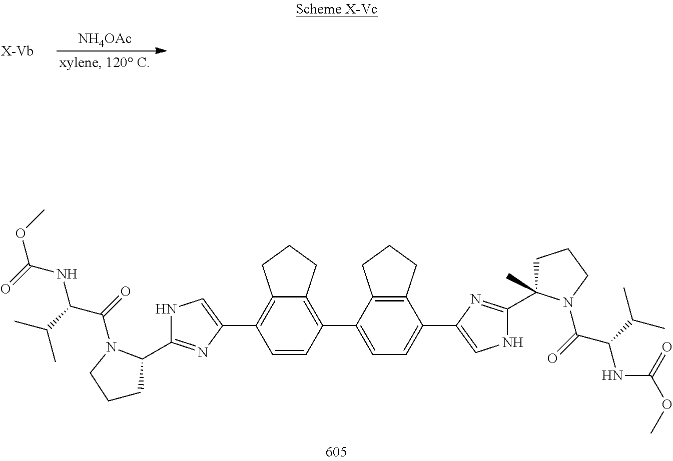

### General Procedure X-AD

A mixture of compound X-Vb (30 mg, 0.03 mmol) and NH₄OAc (77 mg, 1.0 mmol) in xylene (10 mL) was stirred overnight at 120° C. in a sealed tube. After being cooled to r.t., the solvent was removed under reduce pressure and the residue was diluted with EtOAc (30 mL), and washed with water and brine. The organic layers were separated, dried over anhydrous Na₂SO₄ and concentrated under reduced pressure. The residue was purified by Prep-HPLC to afford compound 605 (6.5 mg, yield 23%). ¹H NMR (300 MHz, CD₃OD): δ 7.47-7.51 (m, 2H), 7.05-7.11 (m, 4H), 5.17-5.21 (m, 1H), 4.19-4.23 (m, 2H), 3.87-4.02 (m, 4H), 3.64 (s, 6H), 3.04-3.12 (m, 4H), 2.73-2.76 (m, 4H), 2.58-2.61 (m, 1H), 2.20-2.35 (m, 3H), 2.02-17 (m, 10H), 1.86 (s, 3H), 0.86-0.95 (m, 12H).

### Section XII

### Preparation of Compounds: Section XII

### Example XII-I

### Preparation of substituted hexahydroindolizin-5(1H)-one intermediates

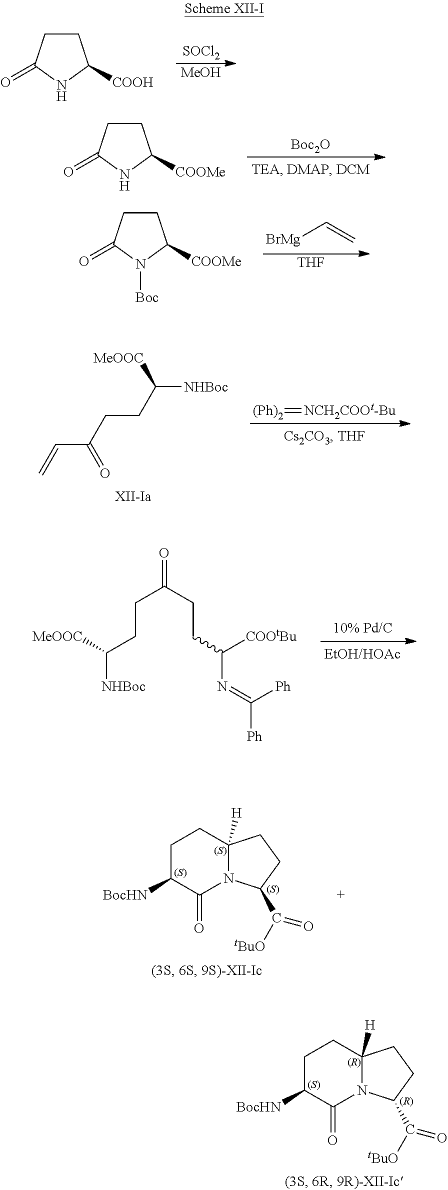

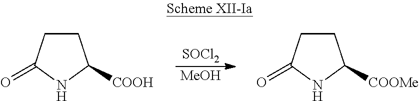

### General Procedure XII-A

To a stirred solution of L-Pyroglutamic acid (15 g, 0.12 mol) in MeOH (120 mL) was added dropwise thionyl chloride (30 mL, 0.16 mmol) at 0° C. under nitrogen. After the addition, the reaction mixture was warmed slowly to the room temperature, and stirred for 2 hours. After concentrated under reduced pressure, H₂O (100 mL) and EtOAc (200 mL) was added. The organic layer was separated, washed with the brine, dried over Na₂SO₄ and concentrated to afford crude L-Pyroglutamic acid methyl ester (17.6 g, crude yield 100%), which was used in the next step without further purification.

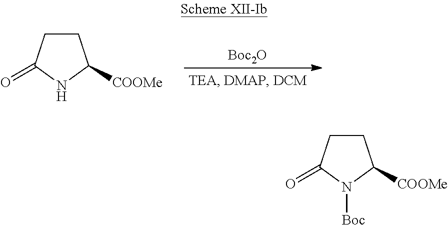

### General Procedure XII-B

To a mixture of L-Pyroglutamic acid methyl ester (17.6 g, 0.12 mol), Et₃N (12.4 g, 0.12 mol), DMAP (15.0 g, 0.12 mol) in DCM (200 mL) was added Boc₂O (53.6 mL, 0.24 mmol) in DCM (100 mL) under nitrogen protection. After the addition, the mixture was stirred for 2 hours at the room temperature. Subsequently, the mixture was concentrated in vacuo. The residue was purified by column chromatography (PE:EA=5:1) to afford Boc-L-pyroglutamic acid methyl ester (10 g, yield 33%).

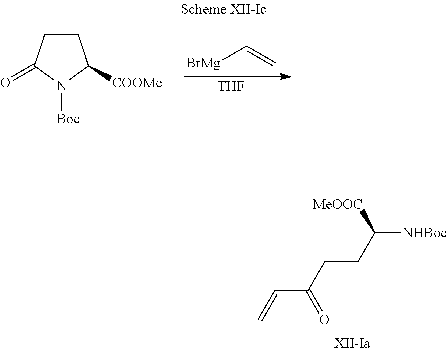

### General Procedure XII-C

To a stirred solution of Boc-L-pyroglutamic acid methyl ester (8.5 g, 34.4 mol) in anhydrous THF (150 mL) was added dropwise vinylmagnesium bromide (42 mL, 42 mmol) at −40° C. under nitrogen protection. After the addition, the mixture was stirred for 3 hours. Then the mixture was treated with AcOH-MeOH (1:1, 50 mL) and diluted with ether. The organic layer was separated and the aqueous layer was extracted with ether (60 mL×3). The combined organic layers were washed with the brine, dried over Na₂SO₄, and concentrated in vacuo. The residue was purified by column chromatography (PE:EA=4:1) on silica gel to afford compound XII-Ia (9.3 g, yield 98%).

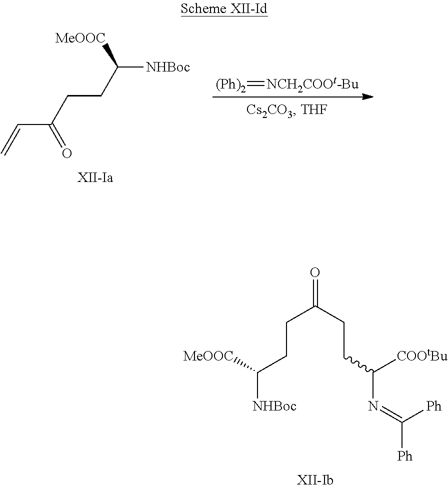

### General Procedure XII-D

To a solution of compound XII-Ia (9.3 g, 34.3 mmol) and N-(diphenylmethylene)glycine tert-butyl ester (11.1 g, 37.6 mmol) in THF (200 mL) was added Cs₂CO₃ (11.2 g, 34.5 mmol). The reaction mixture was stirred at the room temperature overnight under nitrogen protection. After completion of the reaction, the mixture was filtered and the filtrate was concentrated in vacuo. The residue was purified by Prep-HPLC to afford compound XII-Ib (8.8 g, yield 45%). MS (ESI) m/e (M+H)⁺:566.

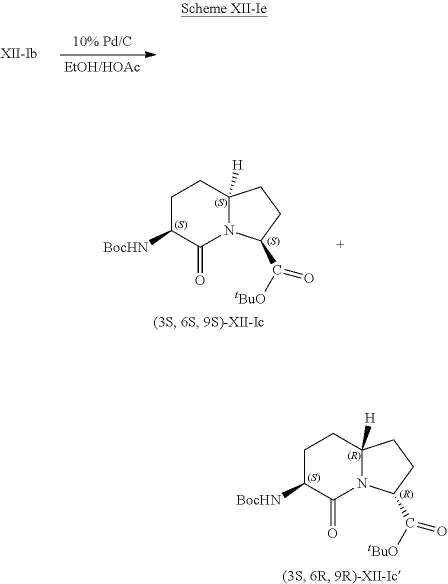

### General Procedure XII-E

Pd/C (1 g, 10%) was added to a solution of compound XII-Ib (8.8 g, 15.5 mmol) in 180 mL of EtOH/HOAc (9/1). The mixture was hydrogenated by hydrogen balloon at room temperature overnight. Subsequently, the catalyst was filtered off through celite, the filtrate was concentrated to give the crude product, which was purified by Prep-HPLC to afford compound (3S, 6S, 9S)-XII-Ic and (3S, 6R, 9R)—XII-Ic′ (total 3.7 g, total yield 57%).

### Example XII-II

### Preparation of Compound 801

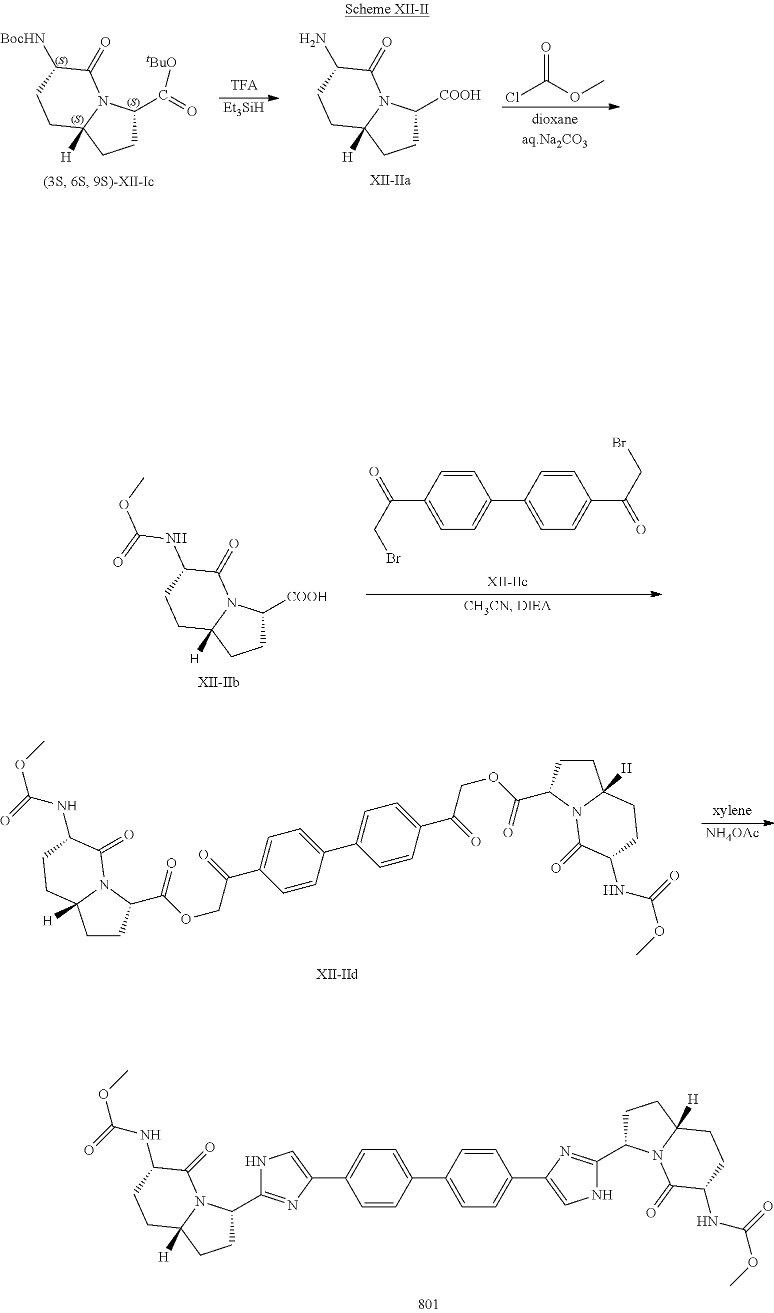

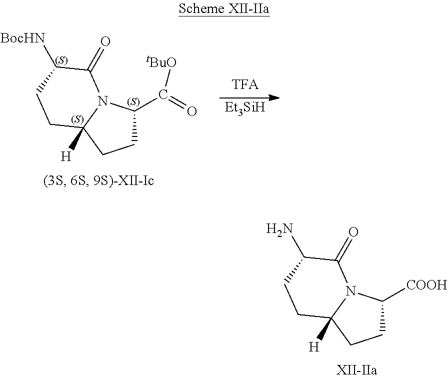

### General Procedure XII-F

A mixture of compound (3S, 6S, 9S)-XII-Ic (2.0 g, 5.64 mmol) in 20 mL of TEA/Et₃SiH (V/V=95/5) was stirred at room temperature for 1 hour under nitrogen protection. The mixture was then concentrated in vacuo to afford crude compound XII-IIa (1.1 g, crude yield 100%), which was used in next step without further purification.

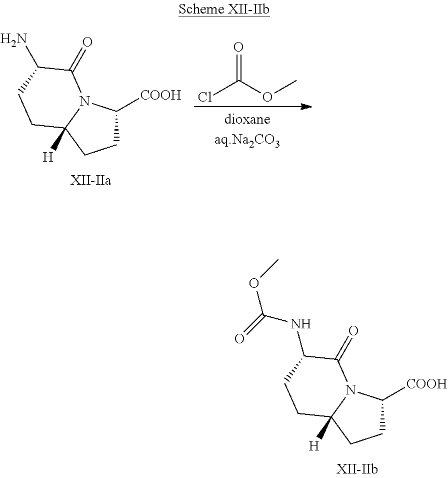

### General Procedure XII-G

Methyl chloroformate (1.0 g, 10.5 mmol) was added to a solution of compound XII-IIa (2 g, 10.1 mmol) in dioxane (20 mL), followed by 10% aqueous Na₂CO₃ (20 mL). The resulting mixture was stirred at room temperature overnight. The mixture was then concentrated in vacuo. The residue was extracted with EtOAc (30 mL×3). The aqueous was acidified to pH˜2 and extracted with EtOAc. The organic layer was separated, washed with the brine, dried over Na₂SO₄ and concentrated under reduced pressure to afford compound XII-IIb (0.7 g, yield 27%).

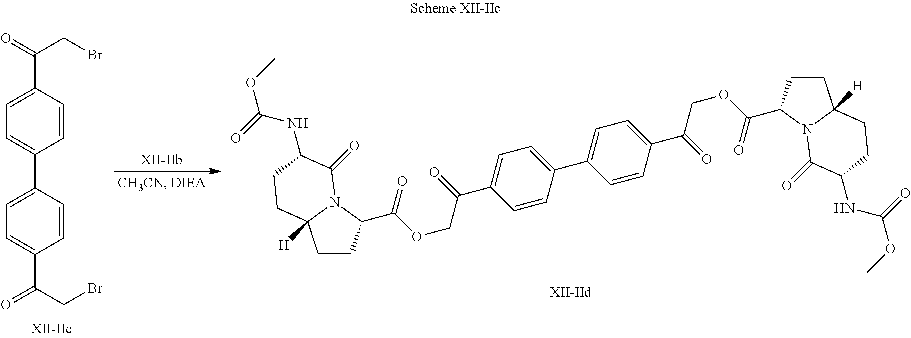

### General Procedure XII-H

To a stirred solution of compound XII-IIc (480 mg, 1.26 mmol) and DIEA (328 mg, 2.54 mmol) in CH₃CN was added compound XII-IIb (650 mg, 2.53 mmol) at 0° C. under nitrogen. After the addition, the mixture was stirred for 0.5 hour, then warmed slowly to room temperature and stirred for another 3 hours. After concentration under reduced pressure, the residue was purified by Prep-HPLC to afford compound XII-IId (130 mg, yield 7%). MS (ESI) m/z (M+H)⁺ 747.

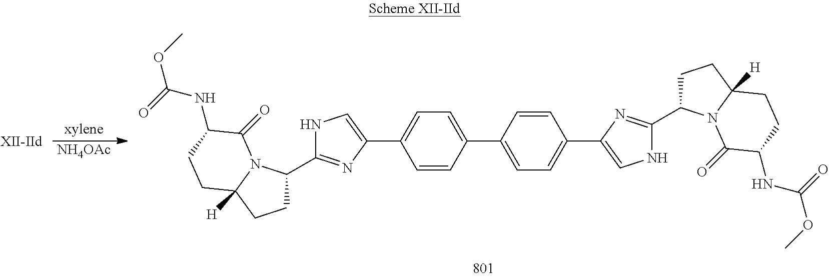

### General Procedure XII-I

A mixture of compound XII-IId (130 mg, 0.17 mmol) and NH₄OAc (322 mg, 4.18 mmol) in xylene (10 mL) was stirred at reflux overnight in a sealed tube. After washing with water, the organic layer was dried over Na₂SO₄, and concentrated. The residue was purified by Prep-HPLC to afford compound 801 (22 mg, yield 17.9%). ¹H NMR (400 MHz, CD₃OD) δ 7.83-7.88 (m, 10H), 5.33 (d, J=8.8 Hz, 2H), 4.11-4.17 (m, 2H), 3.80-3.87 (m, 2H), 3.65 (s, 6H), 2.57-2.60 (m, 2H), 2.15-2.33 (m, 8H) 1.80-2.14 (m, 6H). MS (ESI) m/z (M+H)⁺ 707.3.

### Example XII-III

### Preparation of Compound 802

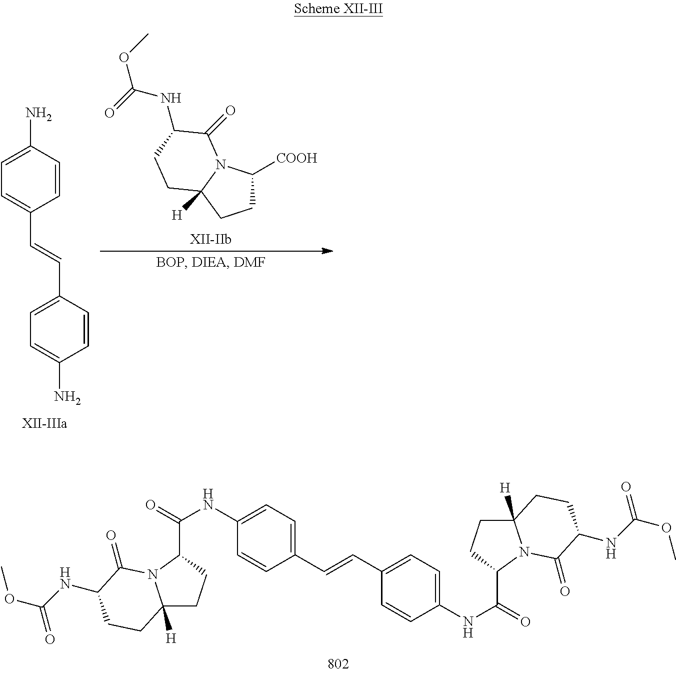

### General Procedure XII-J

To a mixture of compound XII-IIIc (133 mg, 0.46 mmol), compound XII-IIb (240 mg, 0.94 mmol), and DIPEA (420 mg, 3.25 mmol) in DMF (3 mL) was added BOP (415 mg, 0.94 mmol). The resulting mixture was stirred at room temperature for 1 hour. LC-MS indicated the disappearance of compound XII-IIIc. The mixture was washed with water and then extracted with EtOAc. The organic layer was dried over Na₂SO₄ and concentrated under reduced pressure. The residue was purified by Prep-HPLC to afford compound 802 (20 mg, yield 6%). ¹H NMR (400 MHz, CD₃OD) δ 7.58 (d, J=8.4 Hz, 4H), 7.51 (d, J=8.8 Hz, 4H), 7.09 (s, 2H), 4.55 (d, J=8.4 Hz, 2H), 4.25-4.29 (m, 2H), 3.78-3.85 (m, 2H), 3.68 (s, 6H), 2.21-2.28 (m, 4H), 2.10-2.17 (m, 4H), 1.79-1.88 (m, 8H). MS (ESI) m/z (M+H)⁺: 687.3.

### Example XII-IV

### Preparation of Compound 803

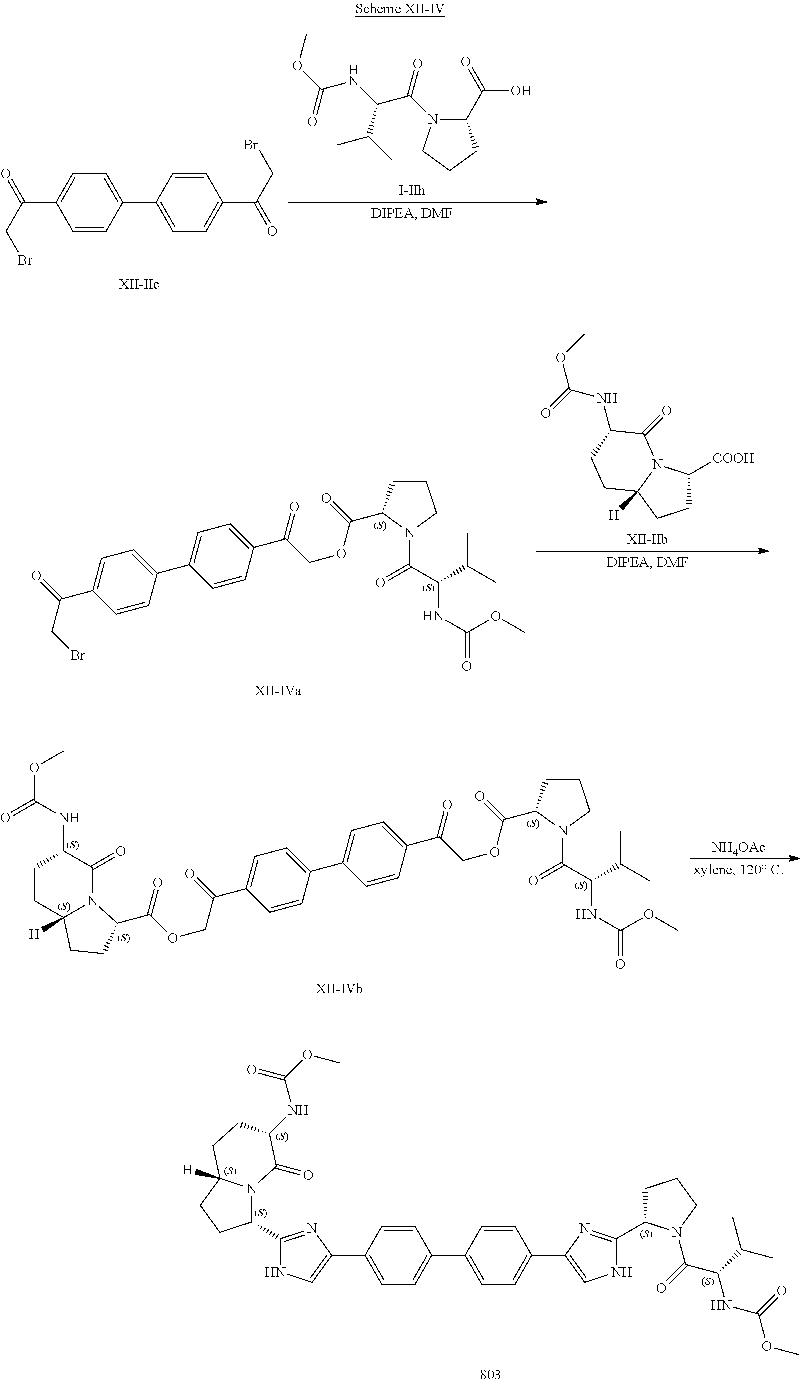

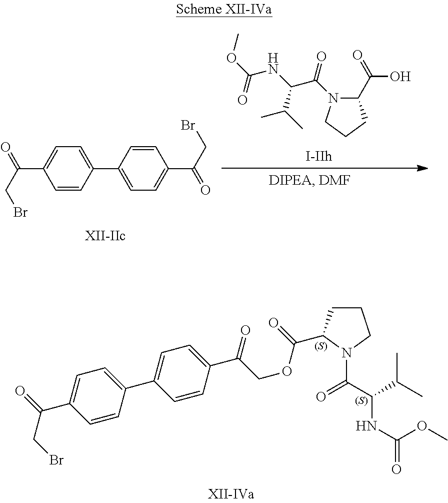

### General Procedure XII-K

Compound XII-IIc (1 g, 2.52 mmol) was dissolved in DMF (15 mL), followed by addition of compound I-IIh (687 mg, 2.52 mmol) and DIPEA (326 mg, 2.52 mmol) at 0° C. The resulting mixture was stirred at 0° C. for 30 min, and then continued stirring for 3 hrs at room temperature. Subsequently, the mixture was diluted with water (30 mL), extracted with EtOAc (30 mL×3). The combined organic layers were washed with brine, dried over sodium sulfate and concentrated in vacuo. The residue was purified by Prep-HPLC to afford compound XII-IVa (567 mg, yield 38%). MS (ESI) m/z (M+H)⁺ 588.

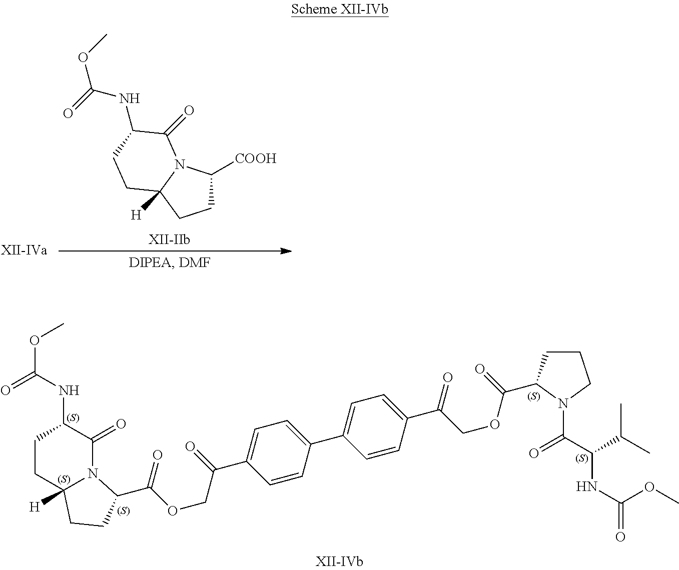

### General Procedure XII-L

Compound XII-IVa (307 mg, 0.52 mmol) was dissolved in DMF (10 mL) and the resulting mixture was treated with compound XII-IIb (134 mg, 2.52 mmol) and DIPEA (67 mg, 0.52 mmol). The mixture was then stirred at room temperature for 3 hrs. Subsequently, the mixture was diluted with water (30 mL), extracted with EtOAc (30 mL×3). The combined organic layers were washed with brine, dried over sodium sulfate and concentrated under reduced pressure. The resulting residue was purified by Prep-HPLC to afford compound XII-IVb (100 mg, yield 25%). MS (ESI) m/z (M+H)⁺ 763.

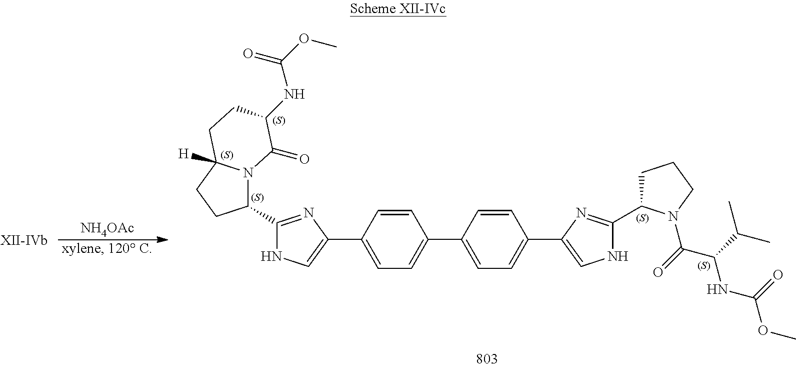

### General Procedure XII-M

A mixture of compound XII-IVb (100 mg, 0.13 mmol) and NH₄OAc (242 mg, 3.14 mmol) in xylene (10 mL) was stirred at 120° C. overnight in a sealed tube. After being cooled to r.t., the solvent was removed under reduced pressure and the residue was diluted with EtOAc (30 mL), washed with water and brine; The organic layer was separated, dried over sodium sulfate and removed under reduced pressure. The resulting residue was purified by Prep-HPLC to afford compound 803 (25 mg, yield 27%). ¹H NMR (300 MHz, CD₃OD) δ 7.84-7.86 (m, 10H), 5.34 (d, J=8.7 Hz, 1H), 5.22-5.27 (m, 1H), 4.23 (d, J=6.9 Hz, 1H), 4.00-4.11 (m, 2H), 3.83-3.88 (m, 2H), 3.65 (s, 3H), 3.64 (s, 3H), 2.27-2.57 (m, 3 H), 2.04-2.25 (m, 10H), 1.85-2.01 (m, 6H). MS (ESI) m/z (M+H)⁺ 723.4.

### Example XII-V

### Preparation of Compound 804

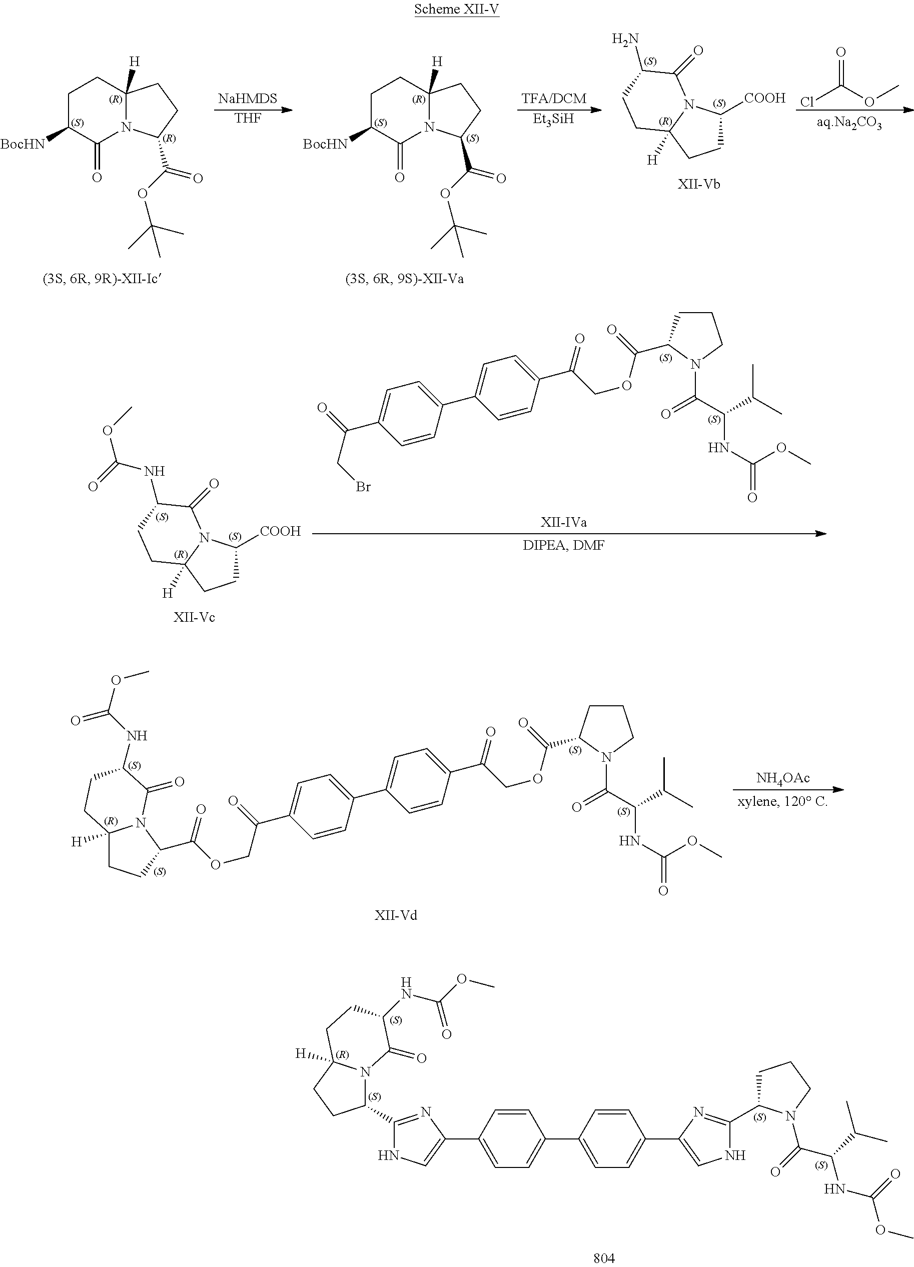

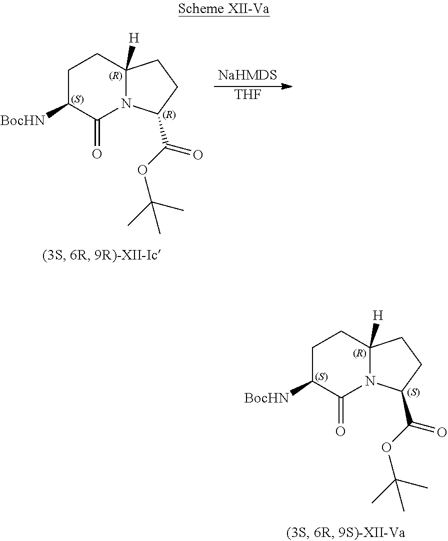

### General Procedure XII-N

To a stirred solution of compound (3S,6R,9R)-XII-Ic′ (1.00 g, 2.8 mmol) in THF was added NaHMDS (2M solution in THF, 2.8 mL, 5.6 mmol) at −78° C. under nitrogen. After the addition, the solution was stirred for 3 hours at −78° C. The reaction was quenched with water and extracted with EtOAc (30 mL×3). The combined organic layers were washed with brine, dried over sodium sulfate and concentrated under reduced pressure. The crude product was purified by Prep-HPLC to afford compound (3S,6R,9S)-XII-Va (490 mg, yield 49%). MS (ESI) m/z (M+Na)⁺ 377.

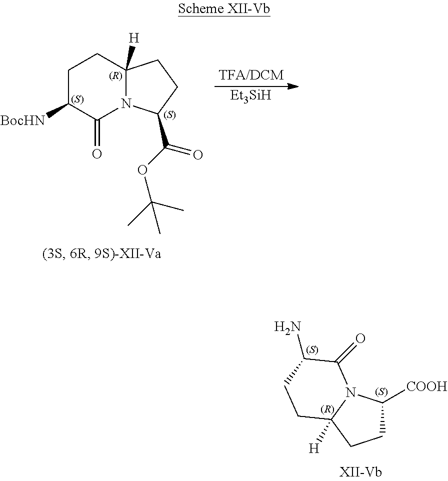

### General Procedure XII-O

To a stirred solution of (3S,6R,9S)-XII-Va (490 mg, 1.38 mmol) in DCM (10 mL) was added TFA (4.65 mL) and Et₃SiH (0.25 mL) at 0° C. under nitrogen. Subsequently, the reaction mixture was stirred at 0° C. for 30 min, and then continued stirring for 3 hrs at room temperature. The solution was concentrated under reduced pressure to afford compound XII-Vb (270 mg, yield 99.0%), which was used in the next step without further purification. MS (ESI) m/z (M+H)⁺ 199.

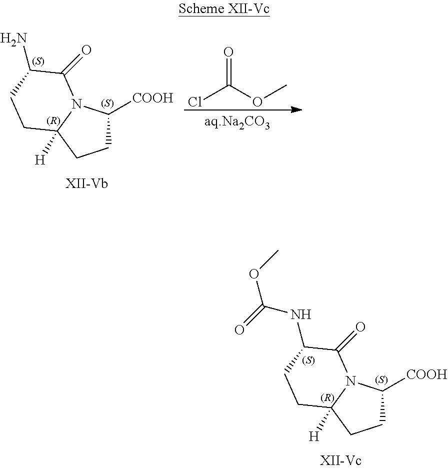

### General Procedure XII-P

Compound XII-Vb (270 mg, 1.36 mmol) was dissolved in dioxane (20 mL) and 10% aqueous Na₂CO₃ (20 mL). To the resulting solution was added methyl chloroformate (145 mg, 1.53 mmol). The reaction mixture was stirred at room temperature overnight. After completion of the reaction, the mixture was concentrated to remove most of the dioxane. The aqueous layer was acidified, and extracted with EtOAc (20 mL×3). The combined organic layers were washed with brine, dried over Na₂SO₄, and concentrated in vacuo to afford compound XII-Vc (180 mg, yield 52%), which was used in next step without further purification.

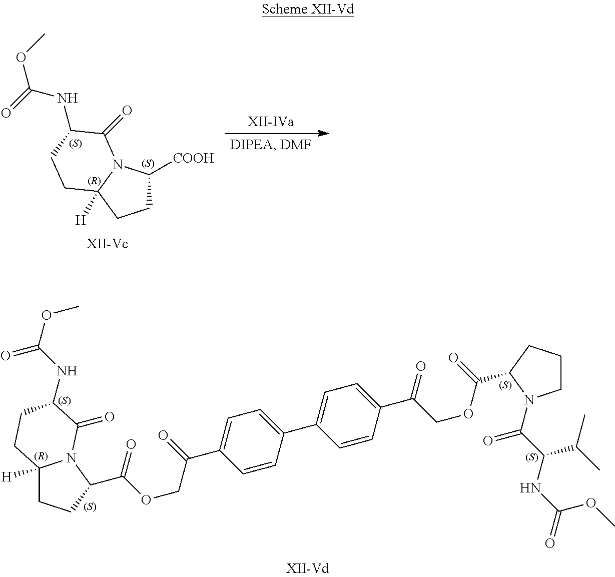

### General Procedure XII-Q

A solution of compound XII-IVa (270 mg, 0.46 mmol) in DMF (10 mL) was treated with compound XII-Vc (117 mg, 0.46 mmol) and DIPEA (59 mg, 0.46 mmol). The resulting mixture was stirred at room temperature for 3 hrs. Subsequently, the mixture was diluted with water (30 mL), and extracted with EtOAc (30 mL×3). The combined organic layers were washed with brine, dried over sodium sulfate and concentrated in vacuo. The resulting residue was purified by Prep-HPLC to afford compound XII-Vd (240 mg, yield 68.5%). MS (ESI) m/z (M+H)⁺ 763.

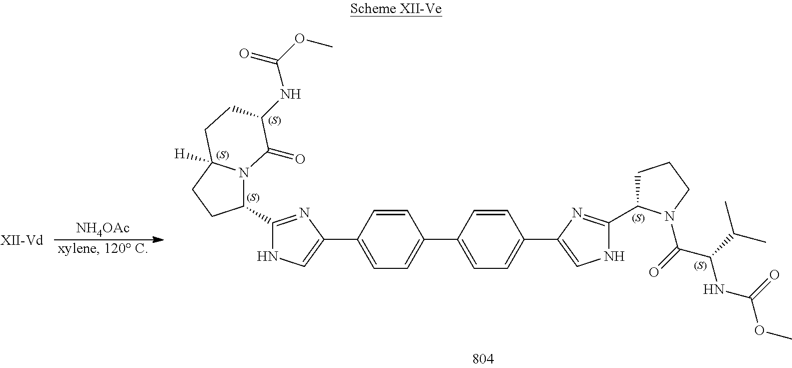

### General Procedure XII-Q

A mixture of compound XII-Vd (240 mg, 0.31 mmol) and NH₄OAc (580 mg, 7.53 mmol) in xylene (10 mL) was stirred at 120° C. overnight in a sealed tube. After cooling to r.t., the solvent was removed under reduce pressure and the remaining residue was diluted with EtOAc (30 mL), and washed with water and brine. The organic layer was dried over sodium sulfate and removed under reduced pressure to give a residue. The residue was purified by Prep-HPLC to afford compound 804 (30 mg, yield 13%). ¹H NMR (400 MHz, CD₃OD) δ 7.84-7.94 (m, 10H), 5.35-5.39 (m, 1H), 5.25-5.29 (m, 1H), 4.25 (d, J=7.2 Hz, 1H), 4.05-4.13 (m, 2H), 3.86-3.93 (m, 2H), 3.68 (s, 3H), 3.65 (d, 3H), 2.57-2.75 (m, 3H), 2.05-2.35 (m, 10H), 1.62-1.81 (m, 6H). MS (ESI) m/z (M+H)⁺ 723.5.

### Example XII-VI

### Preparation of Compound 805

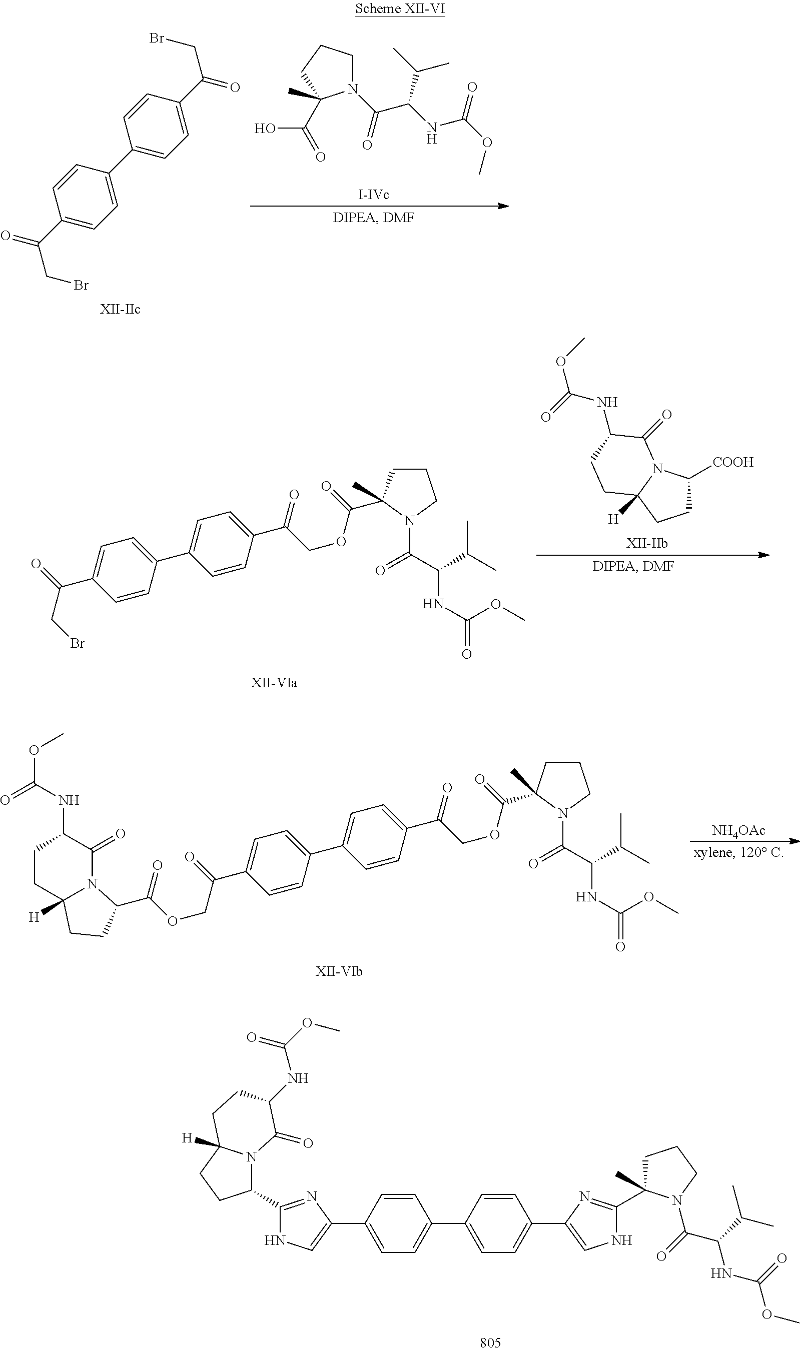

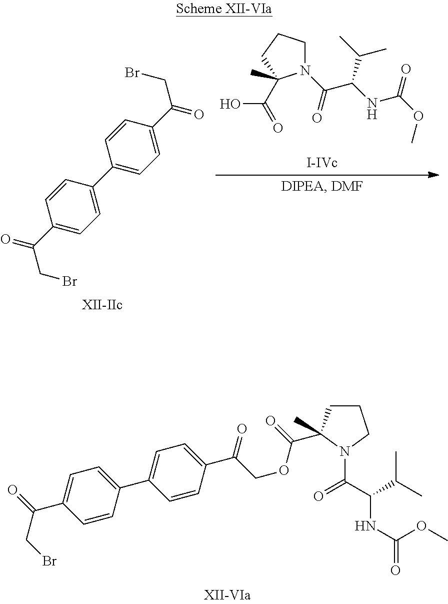

### General Procedure XII-R

A flask was charged with compound XII-IIc (700 mg, 1.76 mmol), compound I-IVc (510 mg, 1.76 mmol), DIPEA (228 mg, 1.76 mmol) and DMF (10 mL). The resulting mixture was stirred at 0° C. for 0.5 hour, and then stirred for 3 hours at room temperature. The mixture was then treated with water (30 mL) and then the resulting aqueous mixture was extracted with EtOAc (30 mL×3). The combined organic layers were washed with brine, dried over Na₂SO₄, and concentrated to give a residue. The residue was purified by Prep-HPLC to afford compound XII-VIa (430 mg, yield 41%). MS (ESI) m/z (M+H)⁺ 601.

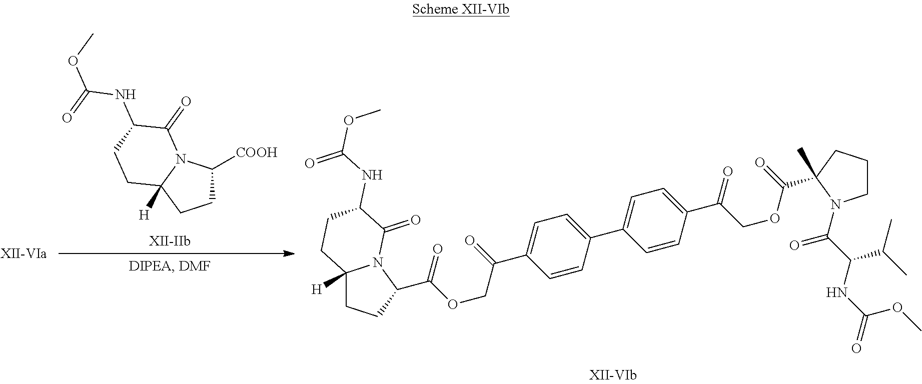

### General Procedure XII-S

A mixture of compound XII-VIa (265 mg, 0.44 mmol), compound XII-IIb (113 mg, 0.44 mmol) and DIPEA (57 mg, 0.44 mmol) in DMF (10 mL) was stirred at room temperature for 3 hrs. Subsequently, water (30 mL) was added to the mixture, and the resulting aqueous mixture was extracted with EtOAc (30 mL×3). The organic layers were combined, dried over Na₂SO₄ and concentrated under reduced pressure to give a residue. The residue was purified by Prep-HPLC to afford compound XII-VIb (140 mg, yield 42%). MS (ESI) m/z (M+H)⁺ 777.

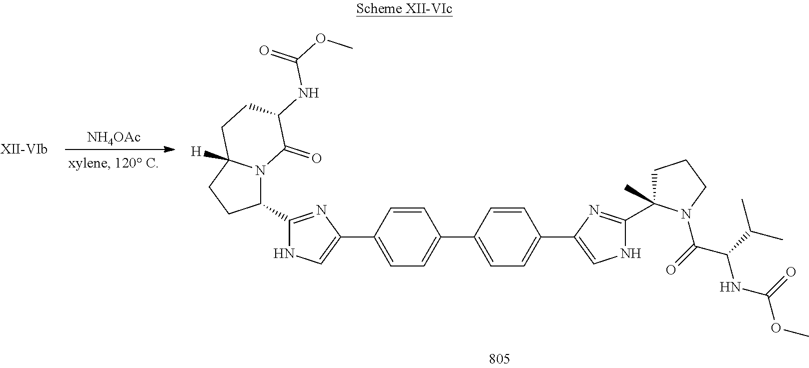

### General Procedure XII-T

A mixture of compound XII-VIb (140 mg, 0.19 mmol) and NH₄OAc (345 mg, 4.48 mmol) in xylene (10 mL) was stirred at 120° C. overnight in a sealed tube. After cooling to r.t., the solvent was removed under reduce pressure and the remaining residue was diluted with EtOAc (30 mL). The organic layer was washed with water and brine, dried over Na₂SO₄, and concentrated under reduced pressure to give a residue. The residue was purified by Prep-HPLC to afford compound 805 (60 mg, yield 44%). ¹H NMR (400 MHz, CD₃OD): δ 7.83-7.86 (m, 10H), 5.32 (d, J=5.7 Hz, H), 4.11-4.16 (m, 3H), 3.91-3.97 (m, 2H), 3.65 (s, 3 H), 3.63 (s, 3H), 2.13-2.54 (m, 9H), 1.81-2.07 (m, 7H), 0.92 (d, J=6.9 Hz, 3H), 0.86 (d, J=6.9 Hz, 3H) MS (ESI) m/z (M+H)⁺ 737.5.

### Example XII-VII

### Preparation of Compound 806

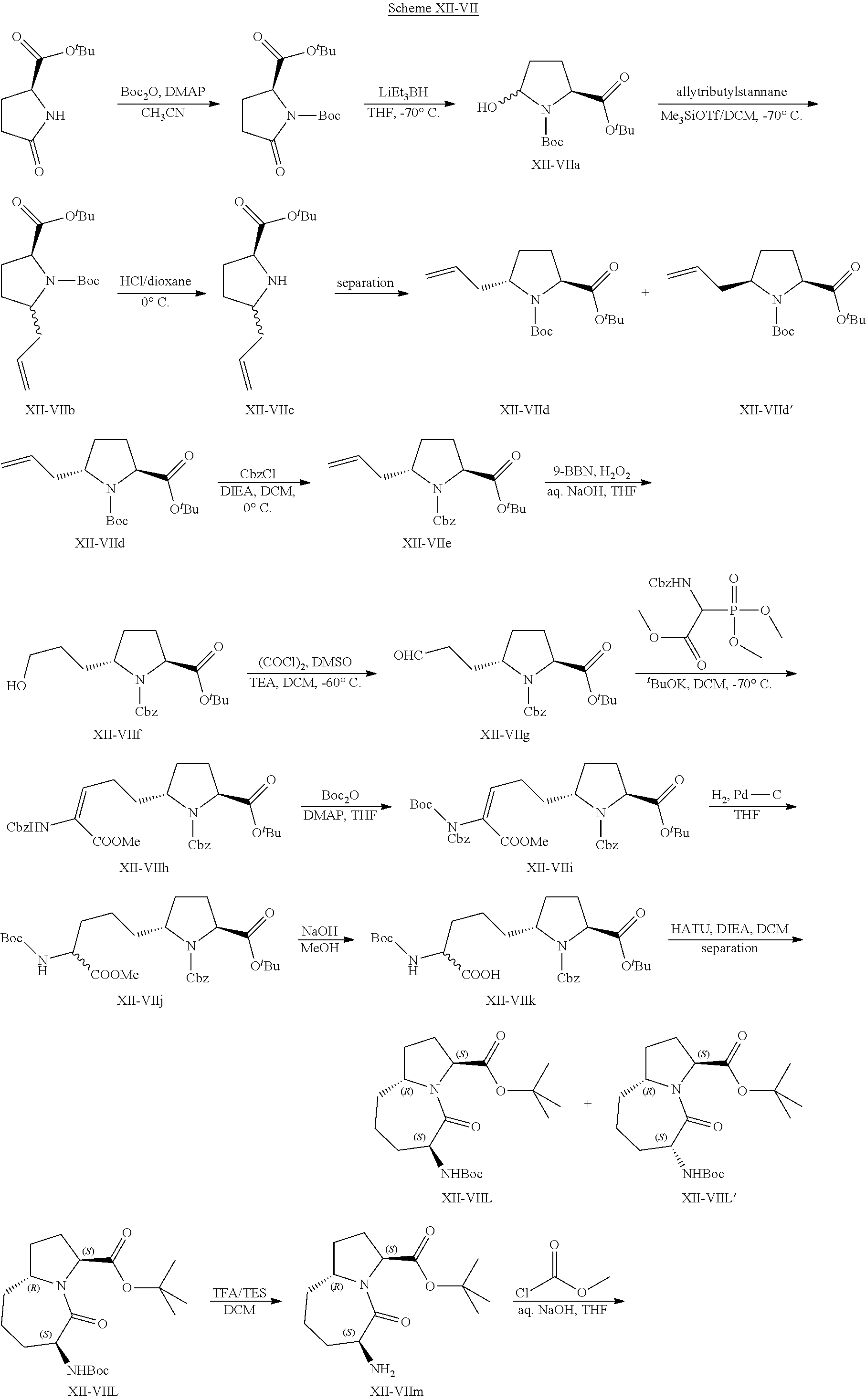

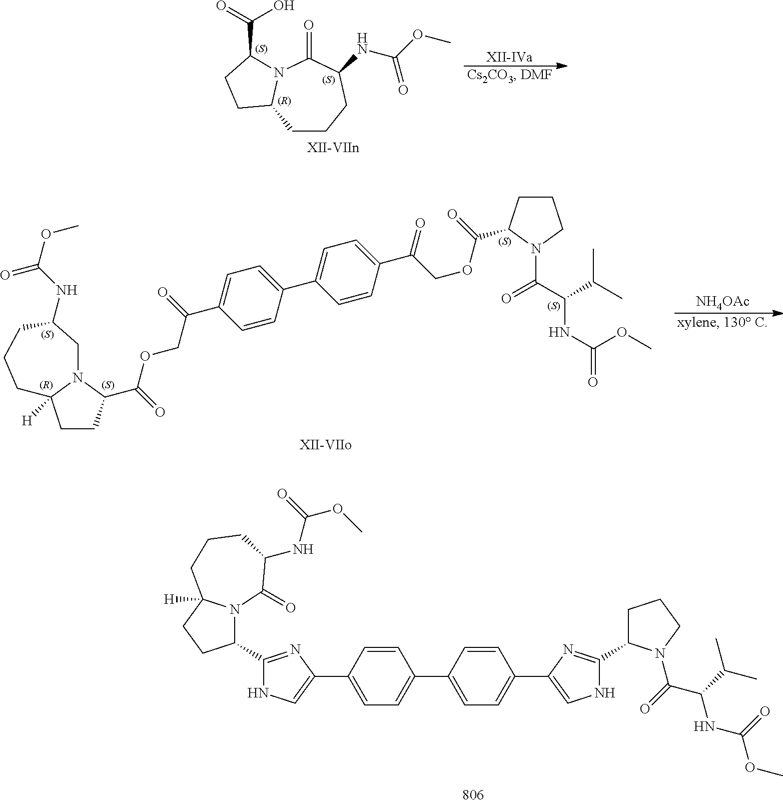

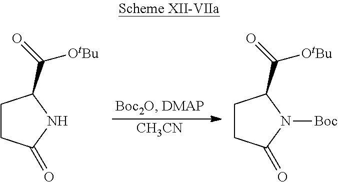

### General Procedure XII-U

Boc₂O (276 g, 1.26 mol) was added to a solution of L-pyroglutamic acid tert-butyl ester (108 g, 0.97 mol) and DMAP (10.8 g, 0.087 mol) in acetonitrile (2 L) dropwise at 0° C. Subsequently, the reaction mixture was warmed to room temperature and stirred for 48 hrs. After concentration, the mixture was diluted with water (500 mL) and extracted with EtOAc (250 mL×3). The combined organic layers were dried over Na₂SO₄ and concentrated under reduced pressure to give a residue. The residue was purified by column chromatography on silica gel (Petroleum ether/EtOAc=20/1) to afford Boc-L-pyroglutamic acid tert-butyl ester (280 g, yield 98%).

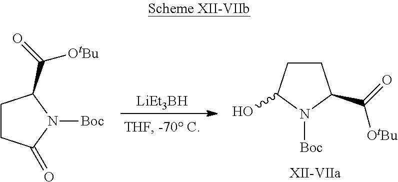

### General Procedure XII-V

Boc-L-pyroglutamic acid tert-butyl ester (100 g, 0.35 mol) was taken up with dry THF (1 L). The resulting mixture was treated with LiEt₃BH (1 M solution in THF, 420 mL, 0.42 mol) dropwise at −70° C. and then the resulting mixture was stirred for 3 hrs. After quenched with saturated aq. NH₄Cl, the aqueous mixture was extracted with EtOAc (500 mL×3). The combined organic layers were washed with brine, dried over Na₂SO₄ and concentrated in vacuo to give crude compound XII-VIIa (100 g, crude yield 100%), which was used in next step without further purification.

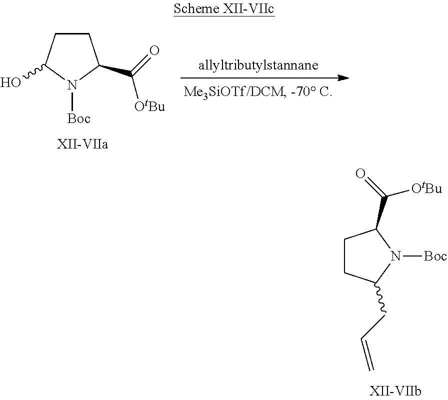

### General Procedure XII-W

To a solution of compound compound XII-VIIa (100 g, 0.35 mol) and allytributyltin (128 mL, 0.42 mol) in dry CH₂Cl₂ (800 mL) was added dropwise of Me₃SiOTf (75 mL, 0.42 mol) with stirring at −70° C. The resulting mixture was stirred for 1 hour at −70° C. and then treated with saturated aq. NH₄Cl solution. The aqueous mixture was extracted with EtOAc (500 mL×3). The combined organic layers were washed with brine, dried over Na₂SO₄ and concentrated under reduced pressure to give a residue. The residue was purified by column chromatography on silica gel (Petroleum ether:EtOAc=3:1) to afford compound XII-VIIb (88 g, yield 81%). MS (ESI) m/e (M+H)⁺:312.2.

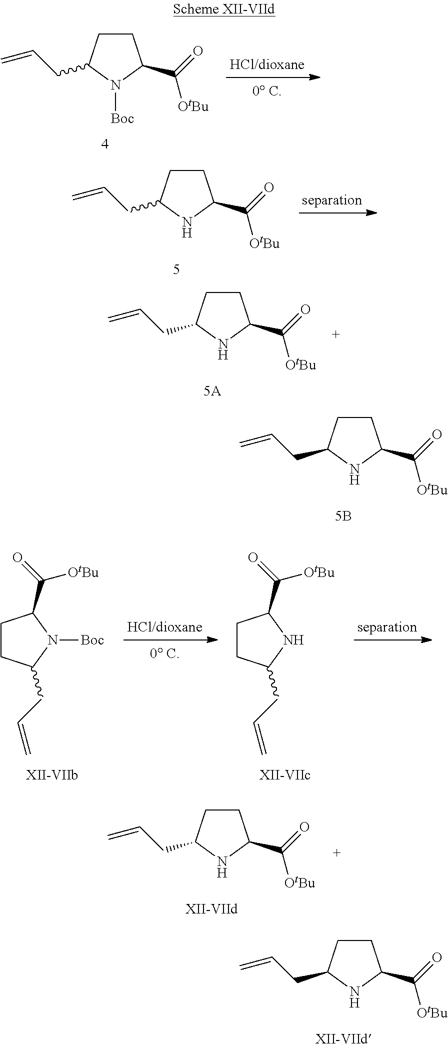

### General Procedure XII-X

A solution of compound XII-VIIb (88 g, 0.28 mol) in HCl/dioxane (500 mL, 4 M) was stirred at 0° C. for 1 hour. After concentration under reduced pressure, the residue was diluted with saturated aq. NaHCO₃, and extracted with EtOAc (250 mL×x3). The combined organic layers were washed with brine, dried over Na₂SO₄ and concentrated under reduced pressure to give crude compound XII-VIII, which was purified by column chromatography on silica gel (Petroleum ether:EtOAc=50:1) to yield compound XII-VIId and compound XII-VIId′ (cis/trans, about 2/1, cis-XII-VIId′, 17 g; trans-XII-VIId, 8 g, total yield 54%).

Compound XII-VIId: ¹H NMR (400 MHz, CDCl₃): δ 5.75-5.84 (m, 1H), 5.01-5.11 (m, 2H), 3.72-3.76 (q, 1H), 3.26-3.30 (m, 1H), 2.37 (brs, 1H), 2.16-2.24 (m, 3H), 1.76-1.90 (m, 2H), 1.40-1.50 (m, 10H). MS (ESI) m/z (M+H)⁺ 212.2.

Compound XII-VIId′: ¹H NMR (300 MHz, CDCl₃): δ 5.76-5.85 (m, 1H), 5.02-5.14 (m, 2H), 3.64-3.69 (m, 1H), 3.12-3.17 (m, 1H), 2.60 (brs, 1H), 2.24-2.32 (m, 2H), 2.12-2.04 (m, 1H), 1.81-1.90 (m, 2H), 1.46 (s, 9H), 1.31-1.49 (m, 1H). MS (ESI) m/z (M+H)⁺ 212.2.

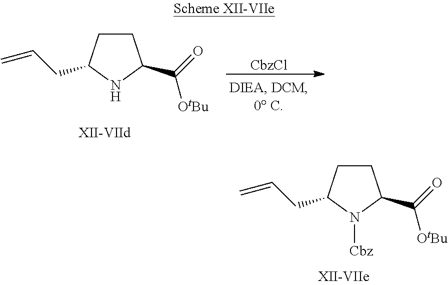

### General Procedure XII-Y

To a solution of compound XII-VIId (7.5 g, 35 mmol) and DIEA (18 g, 140 mmol) in DCM (200 mL) was added Cbz-Cl (12 g, 70 mmol) dropwise at 0° C. The resulting mixture was stirred for 3 hours at 0° C. Subsequently, water (50 mL) was added to the stirring mixture. The layers were partitioned and the organic layer was separated, dried over Na₂SO₄ and concentrated under reduced pressure to give a residue. The residue was purified by column chromatography on silica gel (Petroleum ether:EtOAc=20:1) to afford compound XII-VIIe (10 g, yield 91%).

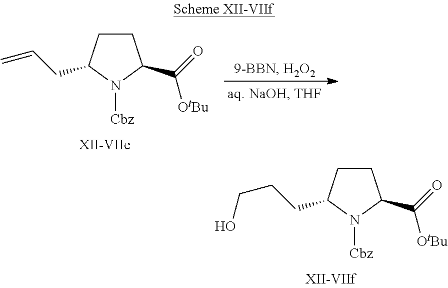

### General Procedure XII-Z

To a stirred solution of compound XII-VIIe (10 g, 29 mmol) in dry THF (200 mL) was added dropwise 9-BBN (139 mL, 69 mmol, 0.5 M in THF). The resulting mixture was stirred for 3 hours at room temperature. Subsequently, the mixture was cooled to 0° C. and water (200 mL) was added dropwise, followed by addition of a solution of NaOH (3 M, 88 mL), and then 30% H₂O₂ (26.5 mL). The resulting mixture was stirred for 1 h at room temperature and then refluxed overnight. After cooling, the aqueous phase was extracted with EtOAc (80 mL×3). The combined organic layers were washed with brine, dried over Na₂SO₄ and concentrated under reduced pressure to give a residue. The residue was purified by column chromatography on silica gel (Petroleum ether:EtOAc=15:1) to afford compound XII-VIIf (9.6 g, yield 91%). MS (ESI) m/z (M+Na)⁺ 364.0.

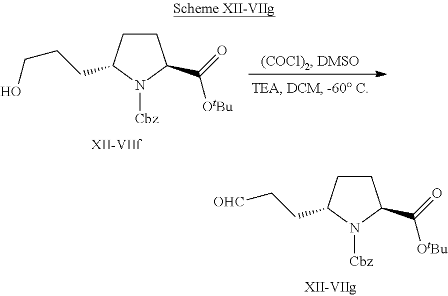

### General Procedure XII-AA

Oxalyl chloride (10 g, 79.2 mmol) was added dropwise to a stirred solution of DMSO (8.3 g, 106 mmol) in CH₂Cl₂ (130 mL) at −60° C. After 30 min the mixture was treated with a solution of compound XII-VIIf (9.6 g, 26.4 mmol) in CH₂Cl₂ (20 mL) dropwise and stirring was continued for 30 min at −60° C. The temperature was maintained during subsequent dropwise addition of TEA (13.2 g, 132 mmol). The resulting mixture was stirred for 1 hour at −60° C. and then was allowed to warm to room temperature for additional 1 hour. The mixture was treated with water (70 mL) and the layers were partitioned. The aqueous phase was extracted with CH₂Cl₂ (80 mL×3). The combined organic layers were washed with brine, dried over Na₂SO₄ and concentrated under reduced pressure to give a crude product. The crude product was purified by column chromatography on silica gel (Petroleum ether:EtOAc=20:1) to afford compound XII-VIIg (7.5 g, yield 79%). MS (ESI) m/z (M+Na)⁺ 384.0.

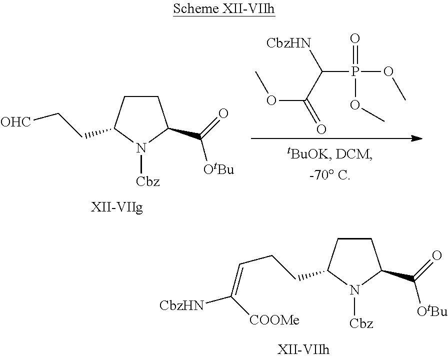

### General Procedure XII-AB

A flask was charged with KOt-Bu (5.6 g, 49.9 mmol) and dry CH₂Cl₂ (60 mL), and the resulting mixture was treated with a solution of Cbz-α-phosphonoglycine trimethyl ester (11 g, 33.2 mmol) in dry CH₂Cl₂ (10 mL) at −70° C. under nitrogen protection. The mixture was stirred for 30 min at this temperature and then treated with a solution of compound XII-VIIg (6 g, 16.6 mmol) in dry CH₂Cl₂ (10 mL). After 5 hrs, the mixture was allowed to warm to room temperature and was treated with water (5 mL). The solvent was evaporated under reduced pressure and the residue was diluted with water (80 mL) and extracted with EtOAc (80 mL×3). The combined organic phases were washed with brine, dried over Na₂SO₄, filtered and concentrated in vacuo. The residue was purified by column chromatography on silica gel (Petroleum ether:EtOAc=20:1) to afford compound XII-VIIh (6.5 g, yield 69%) as a (Z)/(E) diastereoisomeric mixture. MS (ESI) m/z (M+Na)⁺ 589.1.

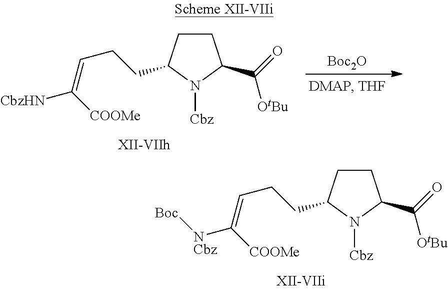

### General Procedure XII-AC

Compound XII-VIIh (6.5 g, 11.5 mmol) was dissolved in dry THF (80 mL) and then treated with Boc₂O (25 g, 11.3 mmol) and a catalytic amount of DMAP (150 mg, 1.2 mmol). The resulting mixture was stirred for 3 hours under nitrogen protection. Subsequently, the mixture was diluted with water (60 mL) and the resulting aqueous mixture was extracted with EtOAc (60 mL×3). The combined organic layers were washed with brine, dried over Na₂SO₄, and concentrated under reduced pressure to give a residue. The residue was purified by column chromatography on silica gel (Petroleum ether:EtOAc=15:1) to afford compound XII-VIIi (7 g, yield 92%). MS (ESI) m/z (M+Na)⁺ 689.1.

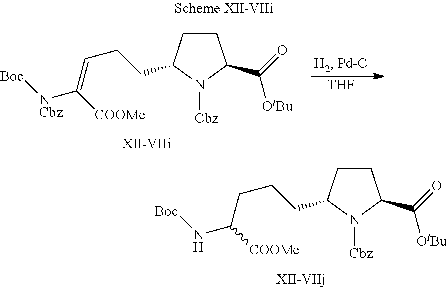

### General Procedure XII-AD

A solution of compound XII-VIIi (7 g, 10 mmol) in THF (100 mL) containing a catalytic amount of 10% Pd/C (1 g) was hydrogenated under H₂ (50 psi). After stirring at room temperature for 20 hours, the catalyst was removed by filtration through celite. The filtrate was concentrated to dryness under reduced pressure and the residue was purified by column chromatography on silica gel (Petroleum ether:EtOAc=5:1) to afford compound XII-VIIj (3.8 g, yield 90%). MS (ESI) m/z (M+H)⁺ 401.1.

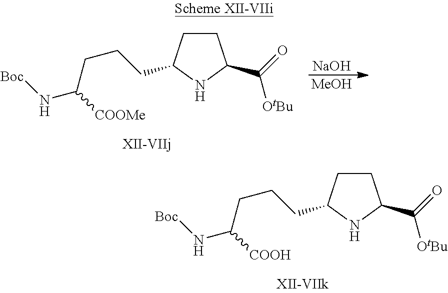

### General Procedure XII-AE

A solution of compound XII-VIIj (3.8 g, 9.5 mmol) in MeOH (80 mL) was treated with aq. NaOH (1 N, 80 mL). After stirring for 1.5 hrs at r.t., the mixture was acidified to pH 3 by treatment with aq. HCl (1 N). The mixture was extracted with CH₂Cl₂ (100 mL×3). The combined organic layers were washed with brine, dried over Na₂SO₄, and concentrated under reduced pressure to give compound XII-VIIk (1.9 g, crude yield 53%), which was used in the next step without further purification. MS (ESI) m/z (M+H)⁺ 387.1.

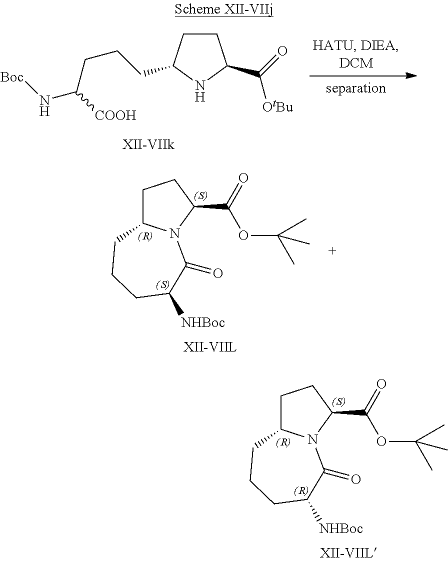

### General Procedure XII-AF

A flask was charged with HATU (2.8 g, 7.4 mmol), DIEA (2.5 g, 19.7 mmol) and DCM (400 mL). The resulting mixture was treated at room temperature with a solution of compound XII-VIIk (0.9 g, 4.9 mmol) in DCM (20 mL) dropwise over a 30 min period of time. The resulting mixture was stirred 1.5 hrs at room temperature. Subsequently, the mixture was washed with water and the organic layer was dried over Na₂SO₄, and concentrated under reduced pressure to give a residue. The residue was purified by column chromatography on silica gel (Petroleum ether:EtOAc=20:1) to afford compound XII-VIIL (0.5 g, yield 28%). ¹H NMR (400 MHz, CDCl₃): δ 5.67 (brs, 1H), 4.43-4.47 (m, 1H), 4.20-4.22 (m, 1H), 3.75 (brs, 1H), 2.25-1.57 (m, 10H), 1.45 (brs 18H). MS (ESI) m/z (M+Na)⁺ 391.0.

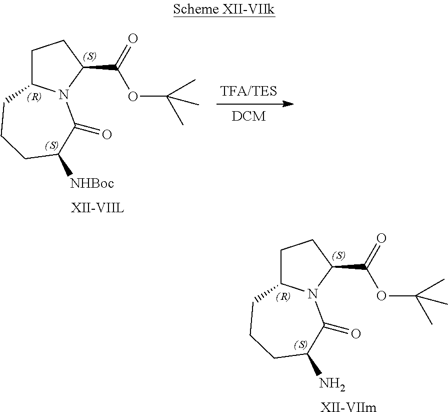

### General Procedure XII-AG

A solution of compound XII-VIIL (470 mg, 1.28 mmol) in DCM (10 mL) was treated with TFA (4.23 mL) and TES (0.23 mL). The resulting mixture was stirred for 1 hr at room temperature. The volatiles were removed under reduced pressure to yield crude compound XII-VIIm (345 mg, crude yield 100%), which was used in the next step without further purification. MS (ESI) m/z (M+H)⁺ 212.9.

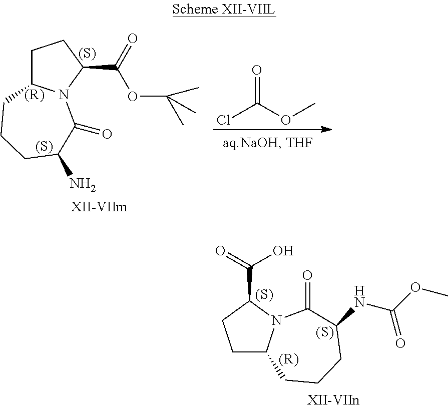

### General Procedure XII-AH

A solution of compound XII-VIIm (270 mg, 1.27 mmol) in THF (10 mL) at room temperature was treated with aq. NaOH (10 mL, 1 M solution) and methyl chloroformate (600 mg, 6.38 mmol). The resulting mixture was stirred for 2 hrs at room temperature. Subsequently, the mixture was acidified to pH 3 with aq. HCl (1 N). The resulting mixture was extracted with CH₂Cl₂ (30 mL×5). The combined organic layers were washed with brine, dried over Na₂SO₄, and concentrated under reduced pressure to give compound XII-VIIn (0.27 g, crude yield 79%), which was used in the next step without further purification. MS (ESI) m/z (M+H)⁺ 270.9.

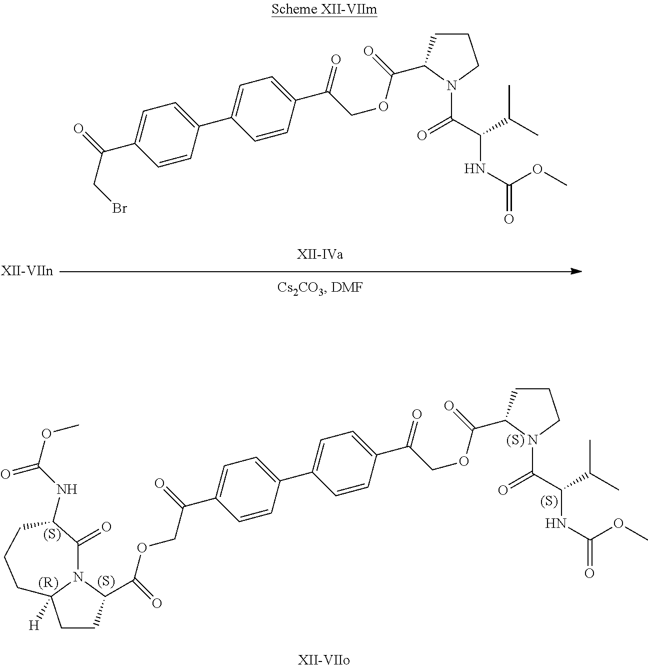

### General Procedure XII-AI

A flask was charged with compound XII-VIIn (80 mg, 0.3 mmol), compound XII-IVa (200 mg, 0.35 mmol), Cs₂CO₃ (200 mg, 0.6 mmol) and DMF (10 mL). The resulting mixture was stirred at room temperature for 3 hours. Subsequently, the mixture was treated with water (10 mL), and the resulting mixture was extracted with EtOAc (30 mL×2). The combined organic layers were washed with brine, dried over Na₂SO₄, and concentrated under reduced pressure to give a residue. The residue was purified by Prep-TLC (Petroleum ether/EtOAc=2/1) to give afford compound XII-VIIo (0.1 g, yield 43%) as yellow solid. MS (ESI) m/z (M+H)⁺ 777.4.

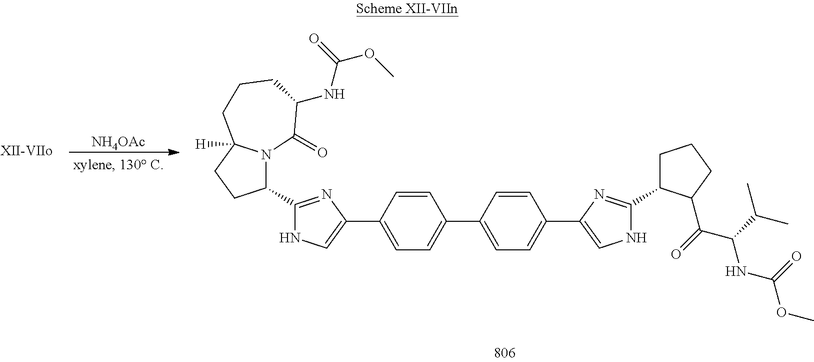

### General Procedure XII-AJ

A mixture of compound XII-VIIo (100 mg, 0.13 mmol) and NH₄OAc (100 mg, 1.3 mmol) in xylene (10 mL) was stirred at 130° C. for 4 hours in a sealed tube. After cooling to r.t., the solvent was removed under reduced pressure to give a residue. The residue was dissolved in DCM (30 mL), and washed with brine. The layers were partitioned and the organic layer was dried over Na₂SO₄ and concentrated under reduced pressure to give a residue. The residue was purified by prep-HPLC to afford compound 806 (10 mg, yield 10%). ¹H NMR (400 MHz, CD₃OD): δ 7.64-7.81 (m, 8H), 7.32-7.38 (m, 2H), 5.34-5.38 (m, 1H), 5.16-5.19 (m, 1H), 4.32-4.38 (m, 1H), 4.19-4.23 (m, 2H), 3.95-4.02 (m, 1H), 3.81-3.87 (m, 1H), 3.75 (s, 3H), 3.65 (s, 3H), 2.30-2.41 (m, 3H), 2.12-2.28 (m, 4H), 1.98-2.06 (m, 3H), 1.59-1.87 (m, 5H), 0.89-0.99 (m, 6H). MS (ESI) m/z (M+H)⁺ 737.2.

### Example XII-VIII

### Preparation of Compound 807

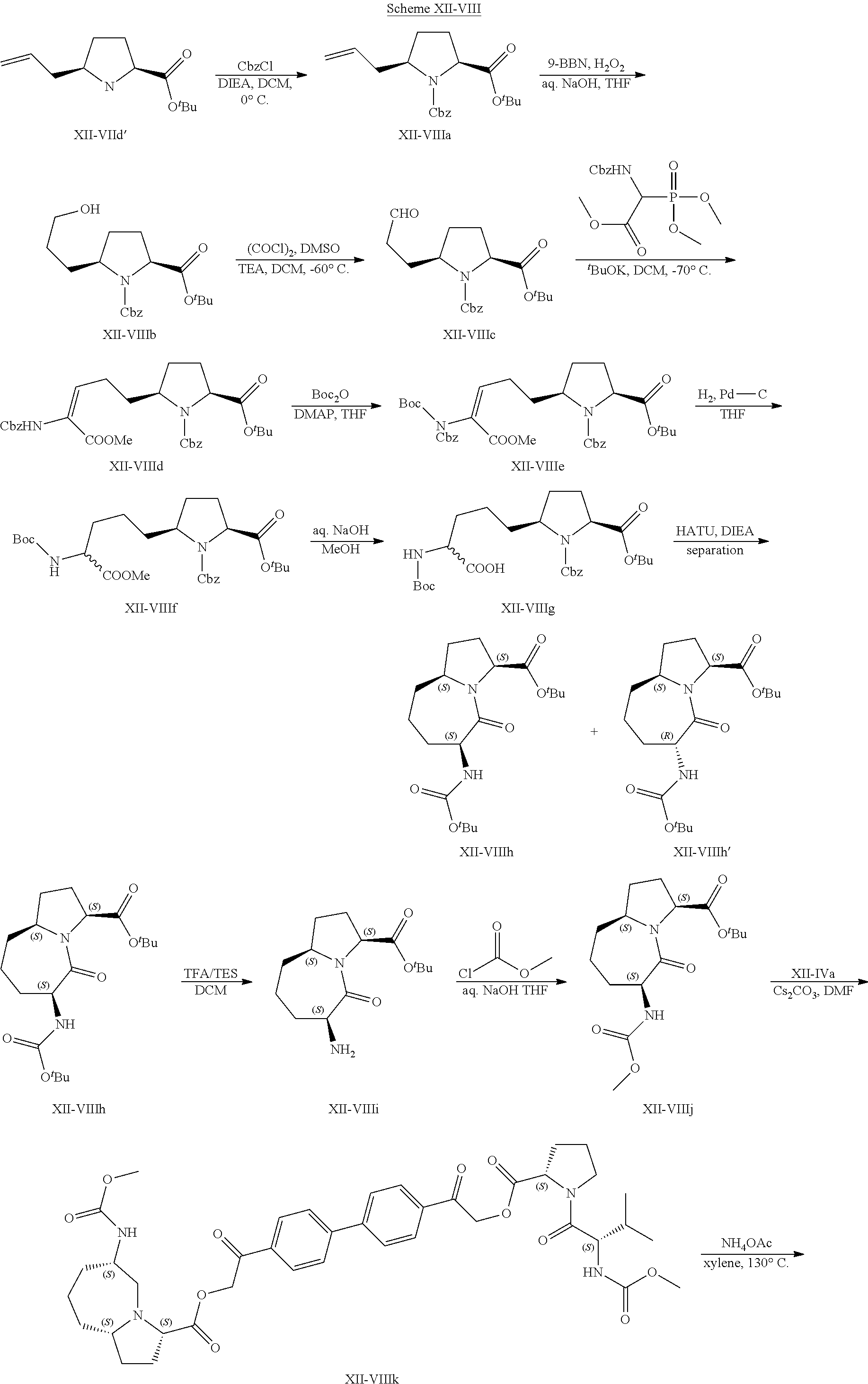

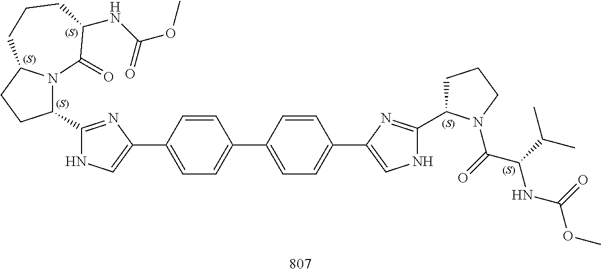

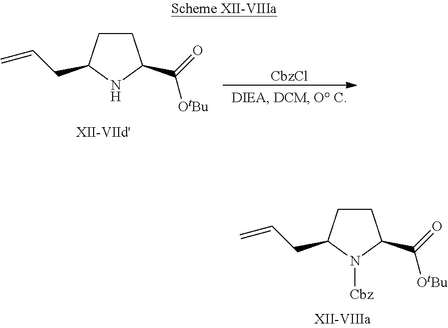

### General Procedure XII-AK

Compound XII-VIId′ (12.6 g, 59.63 mmol) was dissolved in DCM (150 mL), followed by addition of DIEA (30.7 g, 178.9 mmol). The resulting solution was treated with Cbz-Cl (20.3 g, 119.3 mmol) dropwise at 0° C. The resulting mixture was stirred for 3 hours at 0° C. Subsequently, water (50 mL) was added, the organic layer was separated, dried over Na₂SO₄ and concentrated under reduced pressure to give a residue. The residue was purified by column chromatography on silica gel (Petroleum ether/EtOAc=20/1) to afford compound XII-VIIIa (20 g, yield 95%).

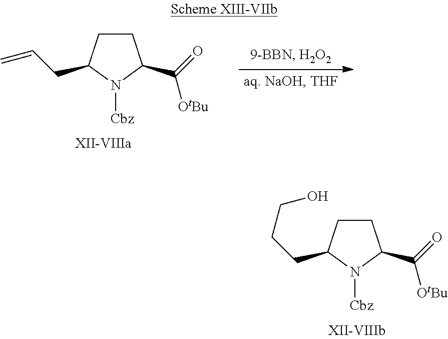

### General Procedure XII-AL

Compound XII-VIIIa (30 g, 86.8 mmol) was taken up with dry THF (600 mL). The resulting solution was treated dropwise with a solution of 9-BBN (350 mL, 173.7 mmol, 0.5 M in THF). The resulting mixture was stirred for 3 hours at room temperature. Subsequently, the mixture was cooled to 0° C., water (300 mL) was added dropwise, followed by addition of a solution of NaOH (3 N, 260 mL) and 30% H₂O₂ (40 mL). The resulting mixture was stirred for additional 1 hour at room temperature and then refluxed overnight. After cooling, the aqueous layer was extracted with EtOAc (200 mL×3). The combined organic layers were washed with brine, dried over Na₂SO₄, and concentrated under reduced pressure to give a residue. The residue was purified by column chromatography on silica gel (Petroleum ether:EtOAc=15:1) to afford compound XII-VIIIb (30 g yield 94%). MS (ESI) m/z (M+H)⁺ 364.0.

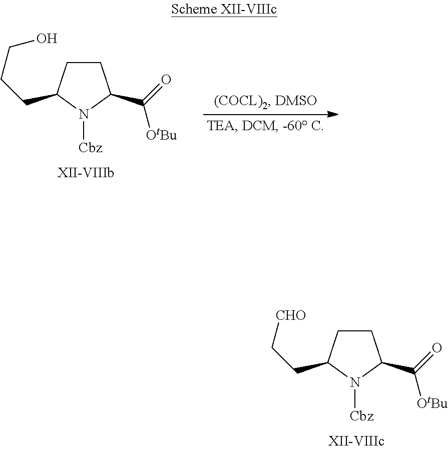

### General Procedure XII-AM

Oxalyl chloride (31.2 g, 0.24 mmol) was added dropwise to a stirred solution of DMSO (25.8 g, 0.33 mmol) in CH₂Cl₂ (500 mL) at −60° C. After 30 min, the resulting mixture was treated dropwise with a solution of compound XII-VIIIb (30 g, 82.5 mmol) in CH₂Cl₂ (50 mL) stirring was continued for 30 min at −60° C. Subsequently, the resulting mixture was treated dropwise with TEA (41.7 g, 0.42 mmol) at −60° C. The resulting mixture was stirred for 1 hour at −60° C. and then was allowed to warm to room temperature for an additional 1 hour. Subsequently, the mixture was treated with water (100 mL) and the layers were partitioned. The aqueous layer was extracted with CH₂Cl₂ (200 mL×3) and then the combined organic layers were washed with brine, dried over Na₂SO₄, and concentrated under reduced pressure. The crude product was purified by column chromatography on silica gel (Petroleum ether:EtOAc=20:1) to afford compound XII-VIIIc (20 g, yield 68%). MS (ESI) m/z (M+Na)⁺ 384.0.

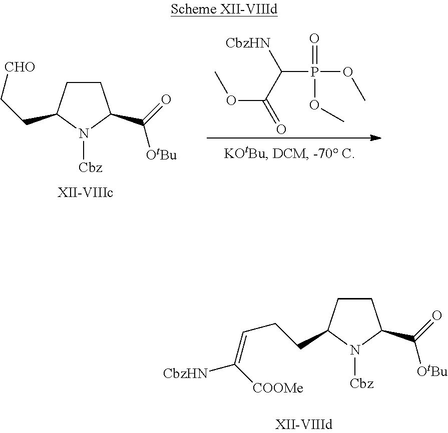

### General Procedure XII-AN

A flask was charged with KOt-Bu (9.3 g, 83 mmol) and dry CH₂Cl₂ (150 mL), and then a solution of Cbz-α-phosphonoglycine trimethyl ester (18.3 g, 55.3 mmol) in dry CH₂Cl₂ (20 mL) was added thereto at −70° C. under nitrogen. The resulting mixture was stirred for 30 min at this temperature and then treated with a solution of compound XII-VIIIc (10 g, 27.66 mmol) in dry CH₂Cl₂ (20 mL). After 5 hrs, the mixture was allowed to warm to room temperature and treated with water (10 mL). Subsequently, the solvent was removed under reduced pressure and the residue was combined with water (200 mL). The aqueous mixture was extracted with EtOAc (200 mL×3). The combined organic phases were washed with brine, and dried over Na₂SO₄. The solid was removed by filtration and the filtrate was removed in vacuo to give a residue. The residue was purified by column chromatography on silica gel (Petroleum ether:EtOAc=20:1) to afford compound XII-VIIId (11.5 g, yield 73%) as a (Z)/(E) isomeric mixture. MS (ESI) m/z (M+Na)⁺ 589.1.

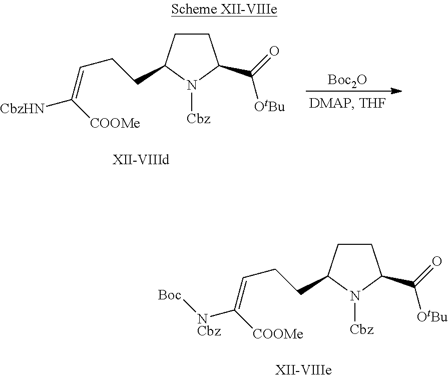

### General Procedure XII-AO

Compound XII-VIIId (11.5 g, 20.3 mmol) was dissolved in dry THF (100 mL). To the resulting solution was added Boc₂O (44.2 g, 203 mmol) and a catalytic amount of DMAP (250 mg, 0.1 mmol). The resulting mixture was stirred for 3 hours under nitrogen protection. The mixture was then diluted with water (100 mL) and extracted with EtOAc (80 mL×3). The combined organic layers were washed with brine, dried over Na₂SO₄, and concentrated under reduced pressure to give a residue. The residue was purified by column chromatography on silica gel (Petroleum ether:EtOAc=15:1) to afford compound XII-VIIIe (10 g, yield 74%). MS (ESI) m/z (M+Na)⁺ 689.1.

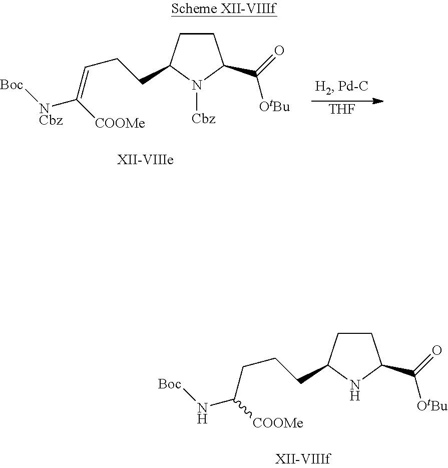

### General Procedure XII-AP

A solution of compound XII-VIIIe (10 g, 15 mmol) in THF (100 mL) containing a catalytic amount of 10% Pd/C (1 g) was hydrogenated under H₂ (50 psi). After stirring at room temperature for 20 hours, the H₂ was replaced with N₂ and then the catalyst was removed by filtration through celite. The filtrate was concentrated to dryness under reduced pressure and the resulting residue was purified by column chromatography on silica gel (Petroleum ether:EtOAc=5:1) to afford compound XII-VIIIf (3.5 g, yield 58%). MS (ESI) m/z (M+H)⁺ 401.1.

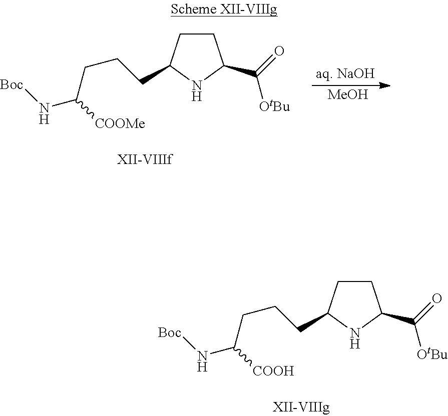

### General Procedure XII-AQ

A solution of compound XII-VIIIf (3.5 g, 8.7 mmol) in MeOH (70 mL) was treated with aq. NaOH (1 N, 70 mL). After stirring for 1.5 hrs at r.t., the mixture was acidified to pH 3 with aq. HCl (1 N) and then the aqueous layer was extracted with CH₂Cl₂ (100 mL×3). The combined organic layers were washed with brine, dried over Na₂SO₄, and concentrated under reduced pressure to afford compound XII-VIIIg (3 g, crude yield 88%), which was used without further purification in the next step. MS (ESI) m/z (M+H)⁺ 387.1.

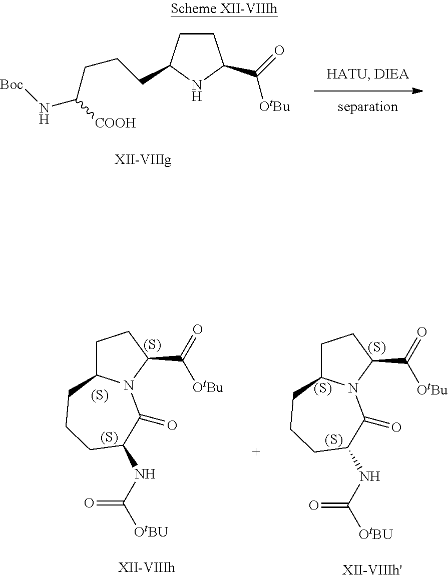

### General Procedure XII-AR

A solution of compound XII-VIIIg (3 g, 7.5 mmol) in DCM (20 mL) was added dropwise over 30 min at room temperature to a flask previously charged with HATU (4.3 g, 11.25 mmol), DIEA (2.9 g, 22.5 mmol) and DCM (700 mL). The resulting mixture was stirred for 1.5 hrs at room temperature. Subsequently, the mixture was washed with water and the layers partitioned. The organic layer was dried over Na₂SO₄, and concentrated under reduced pressure to give a residue. The residue was purified by column chromatography on silica gel (Petroleum ether:EtOAc=20:1) to afford compound XII-VIIIh (1.1 g, yield 78%). ¹H NMR (400 MHz, CDCl₃): δ5.86-5.88 (brs, 1H), 4.41-4.45 (m, 1H), 4.10-4.16 (m, 1H), 3.70-3.78 (brs, 1H), 2.23-1.50 (m, 10H), 1.35-1.39 (brs 18H). MS (ESI) m/z (M+Na)⁺ 391.0.

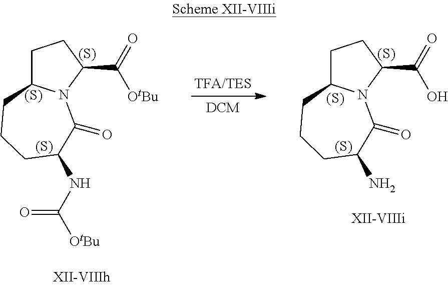

### General Procedure XII-AS

A solution of compound XII-VIIIh (500 mg, 1.3 mmol) in DCM (10 mL) was treated with TFA (4.2 mL) and TES (0.3 mL). The resulting mixture was stirred for 1 hour at room temperature and then concentrated under reduced pressure to afford compound XII-VIIIi (280 mg, crude yield 100%), which was used without further purification in the next step. MS (ESI) m/z (M+H)⁺ 212.9.

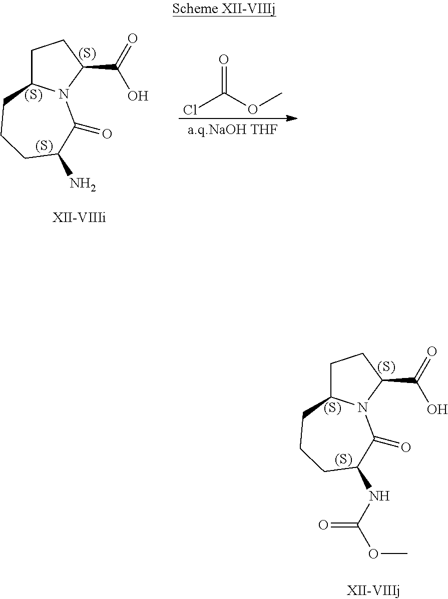

### General Procedure XII-AT

Compound XII-VIIIi (280 mg, 1.3 mmol) was dissolved in THF (5 mL) and the resulting mixture was treated with aq. NaOH (5 mL, 1 M solution) and methyl chloroformate (638 mg, 6.8 mmol) at room temperature. The resulting mixture was stirred for 2 hours at room temperature and then the mixture was acidified to pH 3 with aq. HCl (1 N), and extracted with CH₂Cl₂ (30 mL×5). The combined organic layers were washed with brine, dried over Na₂SO₄, and concentrated under reduced pressure to afford compound XII-VIIIj (0.3 g, crude yield 86%) as white solid, which was used for next step without further purification. MS (ESI) m/z (M+H)⁺ 270.9.

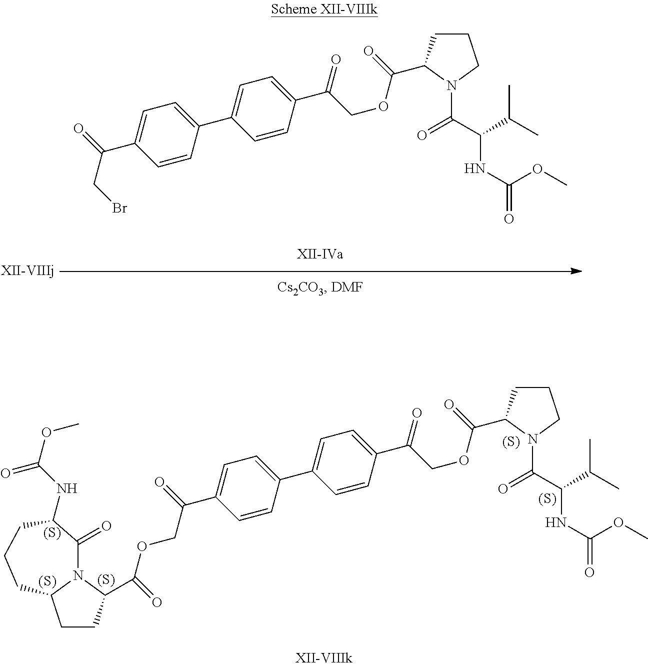

### General Procedure XII-AU

A flask was charged with compound XII-VIIIj (90 mg, 0.33 mmol), compound XII-IVa (234 mg, 0.4 mmol), Cs₂CO₃ (434 mg, 1.33 mmol) and DMF (10 mL). The resulting mixture was stirred at room temperature for 3 hours. Subsequently, water (10 mL) was added, and the mixture was extracted with EtOAc (30 mL×2). The combined organic layers were washed with brine, dried over Na₂SO₄, and concentrated under reduced pressure to give a residue. The residue was purified by Prep-TLC (Petroleum ether/EtOAc=2/1) to afford compound XII-VIIIk (0.1 g, yield 43%) as white solid. MS (ESI) m/z (M+H)⁺ 777.4.

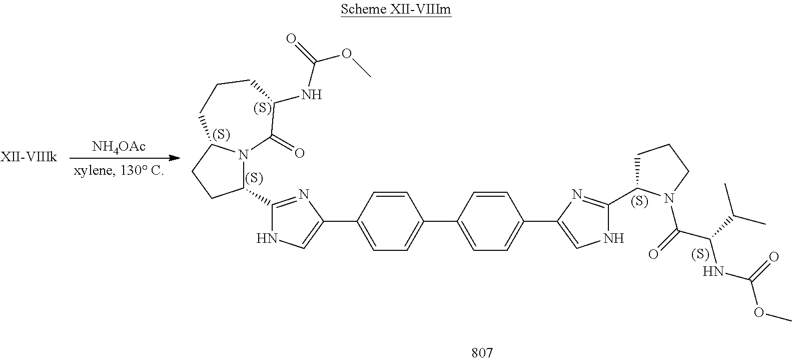

### General Procedure XII-AV

A mixture of compound XII-VIIIk (120 mg, 0.15 mmol) and NH₄OAc (120 mg, 1.5 mmol) in xylene (10 mL) was stirred at 130° C. for 4 hours in a sealed tube. After cooling to r.t., the solvent was removed under reduced pressure to give a residue. The residue was dissolved in DCM (30 mL), and the mixture was washed with brine. The organic layer was separated, dried over Na₂SO₄ and concentrated under reduced pressure to give a residue. The residue was purified by Prep-HPLC to give compound 807 (15 mg, yield 13%). ¹H NMR (400 MHz, CD₃OD): δ 7.55-7.69 (m, 8H), 7.23 (s, 2H), 5.20 (d, J=7.2 Hz, 1H), 5.05-5.08 (m, 1H), 4.20 (d, J=10.8 Hz, 1H), 4.12 (d, J=7.2 Hz, 1H), 3.87-3.91 (m, 2H), 3.75-3.80 (m, 1H), 3.50 (s, 6H), 2.21-2.27 (m, 3H), 2.05-2.11 (m, 3H), 1.92-1.97 (m, 4H), 1.89-1.90 (m, 4H), 1.79-1.83 (m, 1H), 0.79-0.89 (m, 6H). MS (ESI) m/z (M+H)⁺ 737.0.

### Section XVII

HCV Replicon Assay

Huh7 cells containing HCV replicons with an integrated luciferase reporter gene are maintained at 37° C. in 5% CO₂ in Dulbecco's modified Eagle's medium (DMEM; Mediatech, Herndon, Va.) containing 10% heat-inactivated fetal bovine serum (FBS; Mediatech, Herndon, Va.), 2 mM L-glutamine (Cambrex Bioscience, Walkersville, Md.), 1% non essential amino acids (Lonza, Walkersville, Md.), 50 IU/mL penicillin (Mediatech, Herndon, Va.), 50 mg/mL streptomycin (Mediatech, Herndon, Va.) and 0.5 mg/mL G418 (Promega, Madison, Wis.). Cells are sub-divided 1:3 or 4 every 2-3 days.

24 h prior to the assay, Huh7 cells containing sub-genomic HCV replicons are collected, counted, and plated in Nunclon 96-well tissue culture plates (ThermoFisher, Rochester, N.Y.) at 5000 cells/well in 100 mL standard maintenance medium (above) and incubated in the conditions above. To initiate the assay, culture medium is removed, and replaced with 90 mL maintenance media lacking G418. Test compounds are serially diluted three-fold in dimethyl sulfoxide (DMSO) in two duplicate rows for each EC50 determination. These compound solutions are diluted ten-fold in DMEM lacking serum and G418. 10 mL of these compound solutions in media are added to duplicate tissue culture plates. The final volume is 100 μL with a DMSO concentration of 1%. Compound concentrations are adjusted to appropriately define a dose response curve. Typical dilution series range from 100 mM to 1.69 nM final concentrations to 1 nM to 16.9 fM final. Plates are incubated at 37° C. for approximately 48 hr.

Following incubation, media is removed from one of the two duplicate plates and replicon-reporter luciferase activity is measured using a Bright-Glo luciferase assay kit (Promega, Madison, Wis.) according to manufacturer's instructions. Semi-log plots of luciferase activity versus the logarithm of compound concentration are fit to a 4-parameter logistic function using XLfit software (IDBS Inc., Guildford, UK) to determine EC₅₀.

TABLE 20
Examples of activity.

| Compound | EC₅₀ nM |
|---|---|
| 421 | C |
| 422 | C |
| 423 | C |
| 424 | C |
| 501 | A |
| 502 | A |
| 504 | A |
| 505 | A |
| 506 | C |
| 601 | C |
| 602 | C |
| 603 | C |
| 604 | C |
| 605 | C |
| 801 | A |
| 802 | A |
| 803 | C |
| 804 | C |
| 805 | B |
| 806 | C |
| 807 | C |

A indicates an EC₅₀ of 100 nM and greater than 100 nM

B indicates an EC₅₀ between 10 and 100 nM

C indicates an EC₅₀ of 10 nM and less than 10 nM

What is claimed is:

1. A compound having the structure of Formula VII: 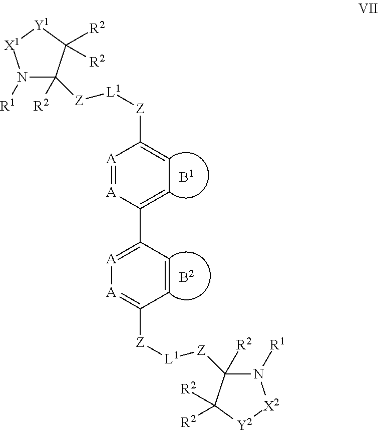
or a pharmaceutically acceptable salt thereof,
wherein:
each R¹ is separately selected from the group consisting of hydrogen, R^{1a}C(═O)— and R^{1a}C(═S)—;
each R^{1a} is separately selected from the group consisting of —C(R^{2a})₂NR^{3a}R^{3b}, alkoxyalkyl, C₁₋₆alkylOC(═O)—, C₁₋₆alkylOC(═O)C₁₋₆alkyl, C₁₋₆alkylC(═O)C₁₋₆alkyl, aryl, aryl(CH₂)ₙ—, aryl(CH₂)ₙO—, aryl(CH═CH)ₘ—, arylalkylO—, arylalkyl, arylOalkyl, cycloalkyl, (cycloalkyl)(CH═CH)ₘ—, (cycloalkyl)alkyl, cycloalkylOalkyl, heterocyclyl, heterocyclyl(CH═CH)ₘ—, heterocyclylalkoxy, heterocyclylalkyl, heterocyclylOalkyl, hydroxyalkyl, R^{c}R^{d}N—, R^{c}R^{d}N(CH₂)ₙ—, (R^{c}R^{d}N)(CH═CH)ₘ—, (R^{c}R^{d}N)alkyl, (R^{c}R^{d}N)C(═O)—, C₁₋₆alkoxy optionally substituted with up to 9 halo, and C₁₋₆alkyl optionally substituted with up to 9 halo, said aryl and heteroaryl each optionally substituted with cyano, halo, nitro, hydroxyl, C₁₋₆alkoxy optionally substituted with up to 9 halo, and C₁₋₆alkyl optionally substituted with up to 9 halo;
each R^{c}R^{d}N is separately selected, wherein R^{c} and R^{d} are each separately selected from the group consisting of hydrogen, alkoxyC(═O)—, C₁₋₆alkyl, C₁₋₆alkylC(═O)—, C₁₋₆alkylsulfonyl, arylalkylOC(═O)—, arylalkyl, arylalkylC(═O)—, arylC(═O)—, arylsulfonyl, heterocyclylalkyl, heterocyclylalkylC(═O)—, heterocyclylC(═O)—, (R^{e}R^{f}N)alkyl, (R^{e}R^{f}N)alkylC(═O)—, and (R^{e}R^{f}N)C(═O)—, wherein the alkyl part of arylalkyl, arylalkylC(═O)—, heterocyclylalkyl, and heterocyclylalkylC(═O)— are each optionally substituted with one R^{e}R^{f}N— group; and wherein the aryl part of arylalkyl, arylalkylC(═O)—, arylC(═O)—, and arylsulfonyl, and the heterocyclyl part of heterocyclylalkyl, heterocyclylalkylC(═O)—, and heterocyclylC(═O)— are each optionally substituted with up to three substituents each independently selected from the group consisting of cyano, halo, nitro, C₁₋₆alkoxy optionally substituted with up to 9 halo, and C₁₋₆alkyl optionally substituted with up to 9 halo;
each R^{e}R^{f}N is separately selected, wherein R^{e} and R^{f} are each separately selected from the group consisting of hydrogen, C₁₋₆alkyl, aryl, arylalkyl, cycloalkyl, (cyclolalkyl)alkyl, heterocyclyl, heterocyclylalkyl, (R^{x}R^{y}N)alkyl, and (R^{x}R^{y}N)C(═O)—;
each R^{x}R^{y}N is separately selected, wherein R^{x} and R^{y} are each separately selected from the group consisting of hydrogen, alkylOC(═O)—, C₁₋₆alkyl, C₁₋₆alkylC(═O)—, aryl, arylalkyl, cycloalkyl, and heterocyclyl;
each C(R^{2a})₂ is separately selected, wherein each R^{2a} is separately selected from the group consisting of hydrogen, C₁₋₆alkyl optionally substituted with up to 9 halo, aryl(CH₂)ₙ—, and heteroaryl(CH₂)ₙ—, said aryl and heteroaryl each optionally substituted with cyano, halo, nitro, hydroxyl, C₁₋₆alkoxy optionally substituted with up to 9 halo, and C₁₋₆alkyl optionally substituted with up to 9 halo, or C(R^{2a})₂ is 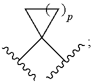
each R^{3a} is separately selected from the group consisting of hydrogen, and optionally substituted C₁₋₆alkyl;
each R^{3b} is separately selected from the group consisting of optionally substituted C₁₋₆alkyl, heteroaryl, —(CH₂)ₙC(═O)NR^{4a}R^{4b}, —(CH₂)ₙC(═O)OR^{5a}, and —(CH₂)ₙC(═O)R^{6a} said heteroaryl optionally substituted with cyano, halo, nitro, hydroxyl, C₁₋₆alkoxy optionally substituted with up to 9 halo, and C₁₋₆alkyl optionally substituted with up to 9 halo;
each R^{4a}R^{4b}N is separately selected, wherein R^{4a} and R^{4b} are each separately selected from the group consisting of hydrogen, optionally substituted C₁₋₆alkyl, and aryl(CH₂)ₙ—;
each R^{5a} is separately selected from the group consisting of optionally substituted C₁₋₆alkyl, and aryl(CH₂)ₙ—;
each R^{6a} is separately selected from the group consisting of optionally substituted C₁₋₆alkyl, and aryl(CH₂)ₙ—;
X¹ is (C(R²)₂)_{q}, 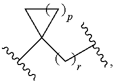
or X¹ is null;
Y¹ is selected from O (oxygen), S (sulfur), S(O), SO₂, NR², and C(R²)₂ with the proviso that when X¹ is null Y¹ is C(R²)₂;
X² is (C(R²)₂)_{q}, 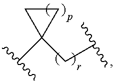
or X² is null;
Y² is selected from O (oxygen), S (sulfur), S(O), SO₂, NR², and C(R²)₂ with the proviso that when X² is null Y² is C(R²)₂;
each R² is separately selected, wherein R² is selected from the group consisting of hydrogen, C₁₋₆alkoxy, C₁₋₆alkyl, aryl, halo, hydroxy, R^{a}R^{b}N—, and C₁₋₆alkyl optionally substituted with up to 9 halo, or optionally two vicinal R² and the carbons to which they are attached are together a fused three- to six-membered carbocyclic ring optionally substituted with up to two C₁₋₆alkyl groups;
each A is separately selected from the group consisting of CR³ and N (nitrogen);
each R³ is separately selected from the group consisting of hydrogen, C₁₋₆alkoxy, C₁₋₆alkylOC₁₋₆alkyl, C₁₋₆alkylOC(═O)—, arylalkylOC(═O)—, —COOH, halo, hydroxy, R^{a}R^{b}N—, (R^{a}R^{b}N)alkyl, (R^{a}R^{b}N)C(═O)—, and C₁₋₆alkyl optionally substituted with up to 9 halo and up to 5 hydroxy;
each L¹ is separately selected from the group consisting of 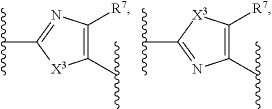
C(═O)(CH₂)ₘOC(═O)—, —C(CF₃)₂NR^{2c}—, and 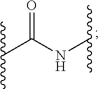
R^{2c} is selected from the group consisting of hydrogen, C₁₋₆alkyl, C₂₋₆alkenyl, C₂₋₆alkynyl, C₃₋₇cycloalkyl, aryl, arylalkyl, heteroaryl, heteroarylalkyl, heterocyclyl, and heterocyclylalkyl, said alkyl optionally substituted with R^{e}R^{f}N—, alkoxy, or C₁₋₆alkylS—;
each X³ is separately selected from the group consisting of NH, NC₁₋₆alkyl, O (oxygen), and S (sulfur);
each R⁷ is separately selected from the group consisting of hydrogen, C₁₋₆alkylOC(═O)—, arylalkylOC(═O)—, —COOH, (R^{a}R^{b}N)C(═O)—, trialkylsilylalkylOalkyl, and C₁₋₆alkyl optionally substituted with up to 9 halo;
each R^{a}R^{b}N is separately selected, wherein R^{a} and R^{b} are each separately selected from the group consisting of hydrogen, C₂₋₆alkenyl, and C₁₋₆alkyl;
each Z is separately selected, wherein Z is selected from the group consisting of O (oxygen) and CH₂, or Z is null;
each m separately is 1 or 2;
each n separately is 0, 1 or 2;
each p separately is 1, 2, 3 or 4;
each q separately is 1, 2, 3, 4 or 5;
each r separately is 0, 1, 2, 3, or 4;
B¹ is a fused optionally substituted saturated or unsaturated three- to seven-membered carbocyclic ring or a fused optionally substituted saturated or unsaturated three- to seven-membered heterocyclic ring, each optionally substituted with one or more R⁴;
B² is a fused optionally substituted saturated or unsaturated three- to seven-membered carbocyclic ring or a fused optionally substituted saturated or unsaturated three- to seven-membered heterocyclic ring, each optionally substituted with one or more R⁴; and
each R⁴ is separately selected from the group consisting of C₁₋₆alkoxy, C₁₋₆alkylOC₁₋₆alkyl, C₁₋₆alkylOC(═O)—, arylalkylOC(═O)—, —COOH, halo, C₁₋₆haloalkyl, hydroxy, R^{a}R^{b}N—, (R^{a}R^{b}N)alkyl, (R^{a}R^{b}N)C(═O)—, and C₁₋₆alkyl optionally substituted with up to 9 halo and up to 5 hydroxy, or optionally two geminal R⁴ are together oxo.

2. The Compound of claim 1,
wherein: 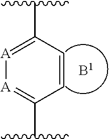
is selected from the group consisting of: 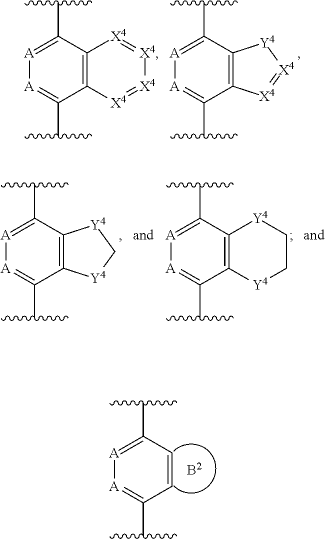
is selected from the group consisting of: 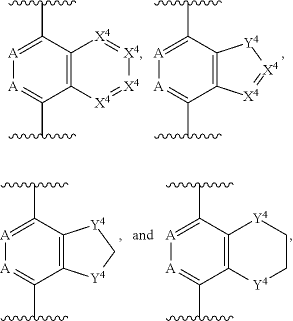
wherein,
each X⁴ is separately selected from the group consisting of CR⁴ and N (nitrogen); and
each Y⁴ is separately selected from the group consisting of C(R⁴)₂, NR⁴, O (oxygen), and S (sulfur).

3. The compound of claim 1, wherein each Z is null.

4. The compound of claim 1 having the structure of Formula VIIa: 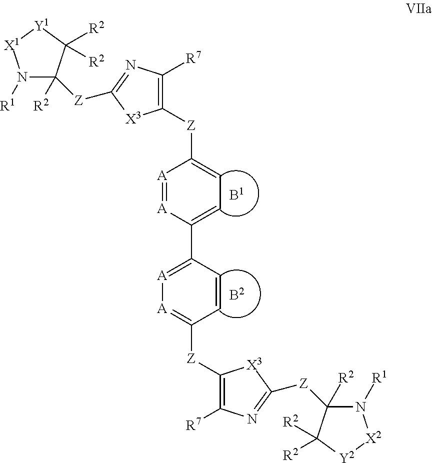
or a pharmaceutically acceptable salt thereof.

5. The compound of claim 1 having the structure of Formula VIIb: 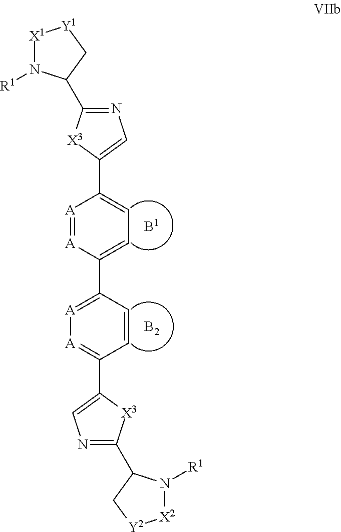
or a pharmaceutically acceptable salt thereof.

6. The compound of claim 5, wherein each R¹ is R^{1a}C(═O)—.

7. The compound of claim 6, wherein each R^{1a} is —CHR^{2a}NHR^{3b}.

8. The compound of claim 7, wherein each R^{2a} is C₁₋₆alkyl;
each R^{3b} is —C(═O)OR⁵; and
each R⁵ is C₁₋₆alkyl.

9. The compound of claim 1, wherein B¹ is a fused saturated or unsaturated three- to seven-membered carbocyclic ring optionally substituted with one or more R⁴.

10. The compound of claim 1, wherein B² is a fused saturated or unsaturated three- to seven-membered carbocyclic ring optionally substituted with one or more R⁴.

11. The compound of claim 1, wherein B¹ is a fused saturated or unsaturated three- to seven-membered heterocyclic ring optionally substituted with one or more R⁴.

12. The compound of claim 1, wherein B² is a fused saturated or unsaturated three- to seven-membered heterocyclic ring optionally substituted with one or more R⁴.

13. The compound of claim 1,
wherein: 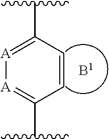
is selected from the group consisting of:

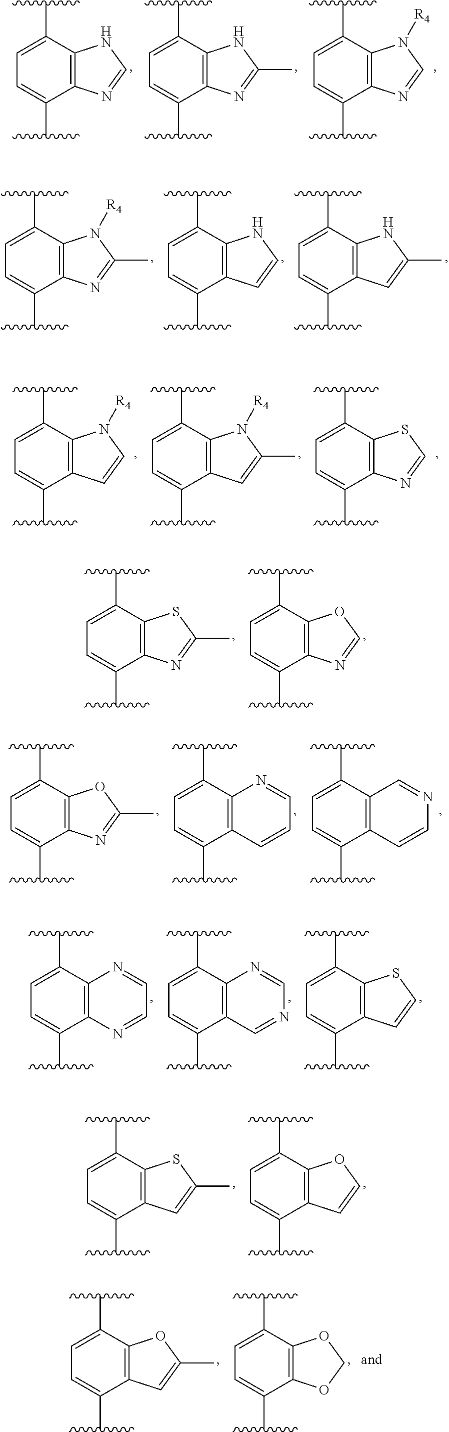

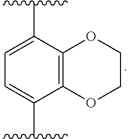

14. The compound of claim 1,
wherein: 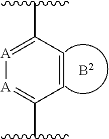
is selected from the group consisting of:

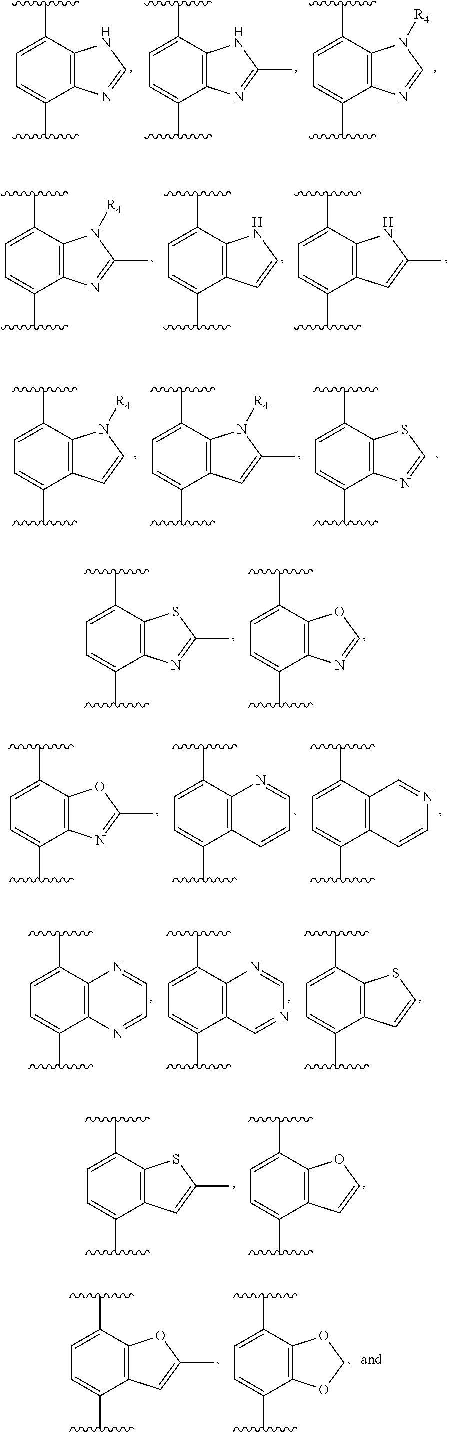

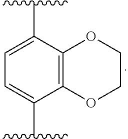

15. The compound of claim 1,
wherein: 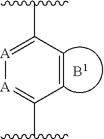
is selected from the group consisting of:

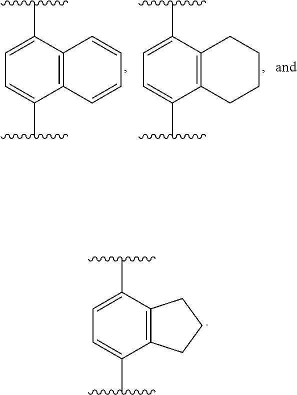

16. The compound of claim 1,
wherein: 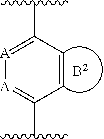
is selected from the group consisting of:

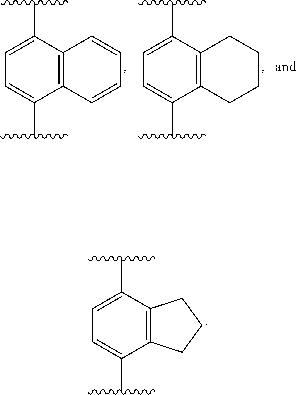

17. The compound of claim 1, having the structure: 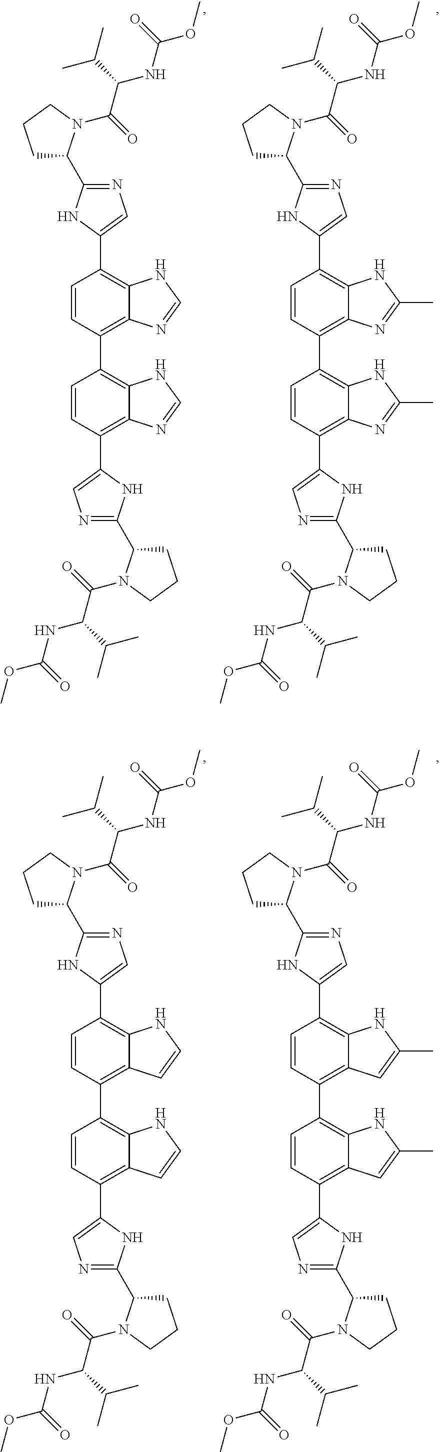 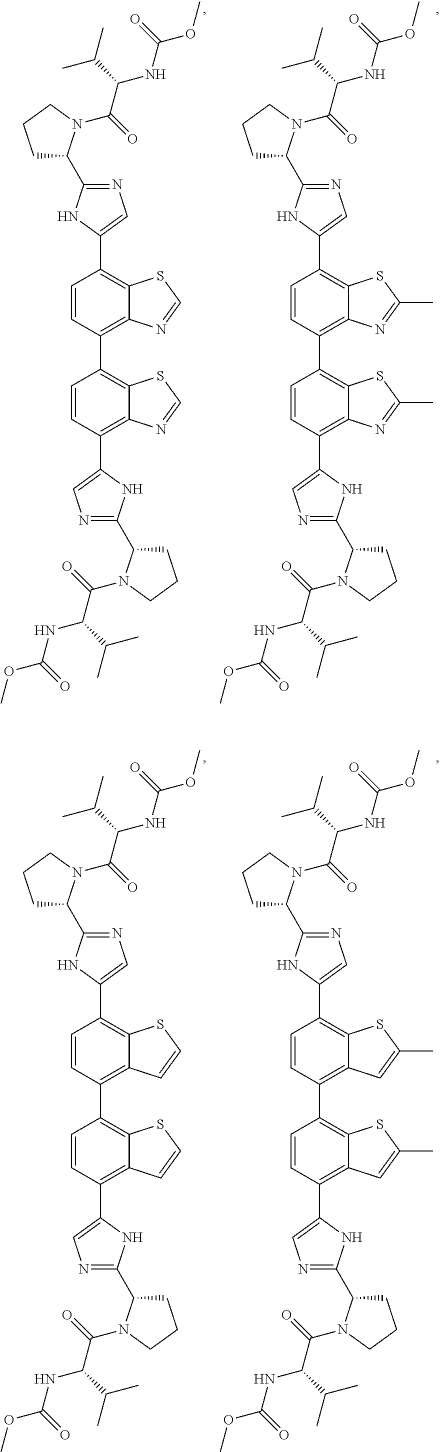 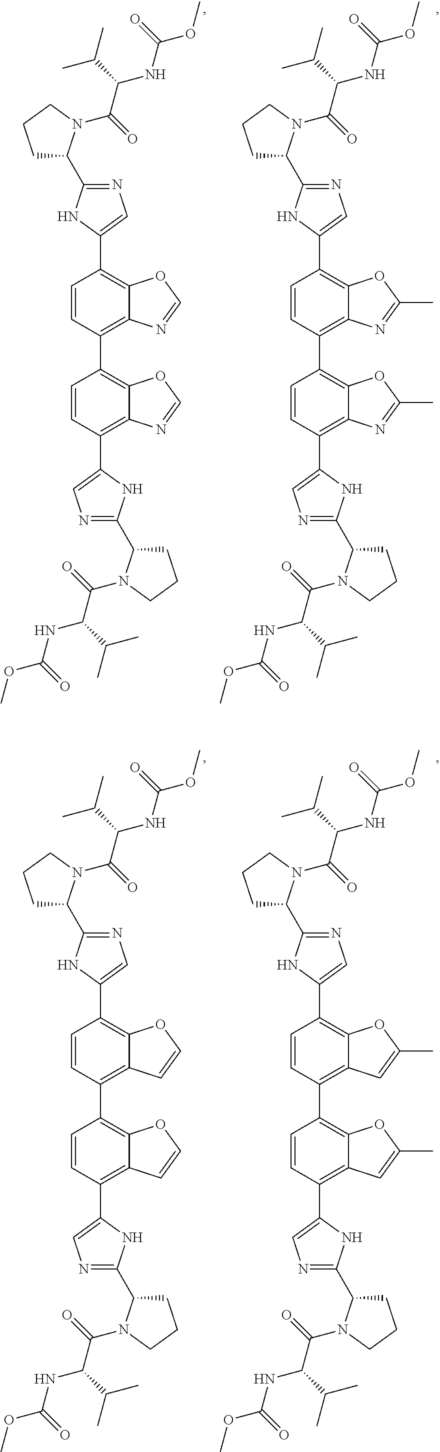 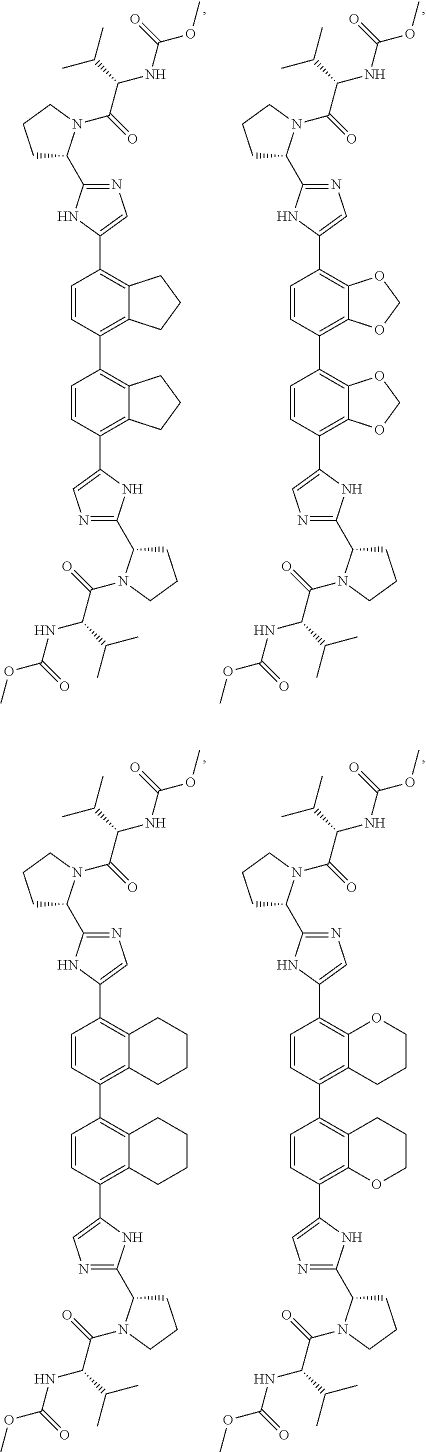 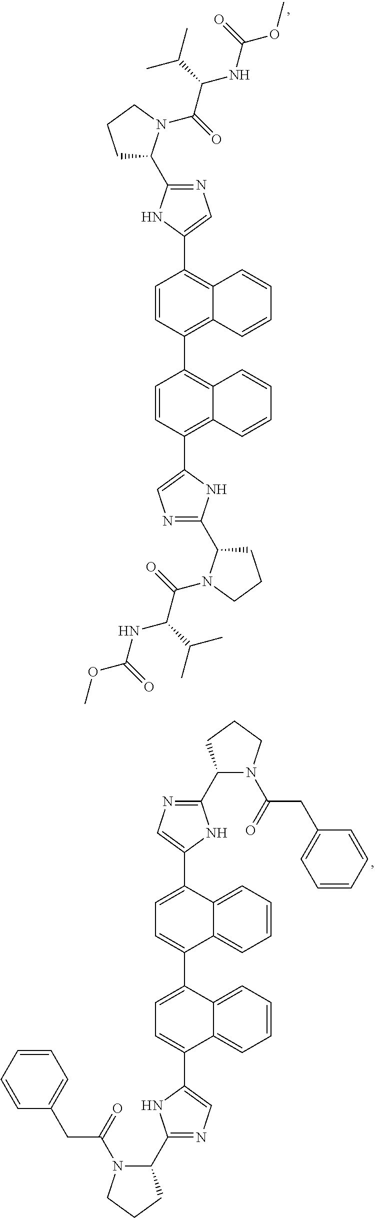 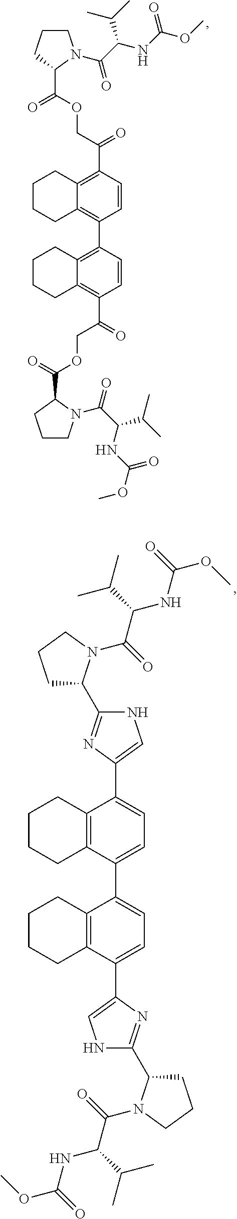 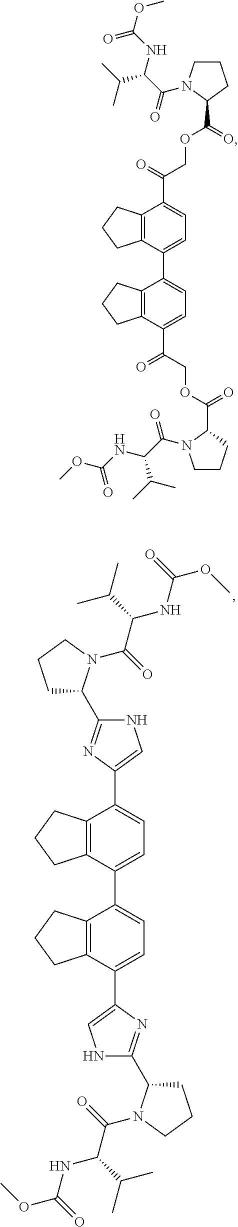 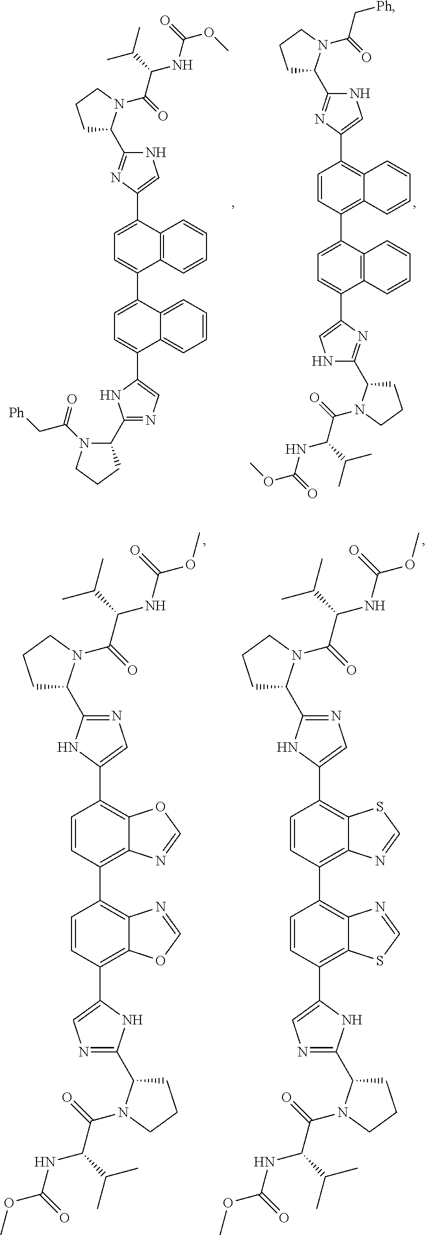 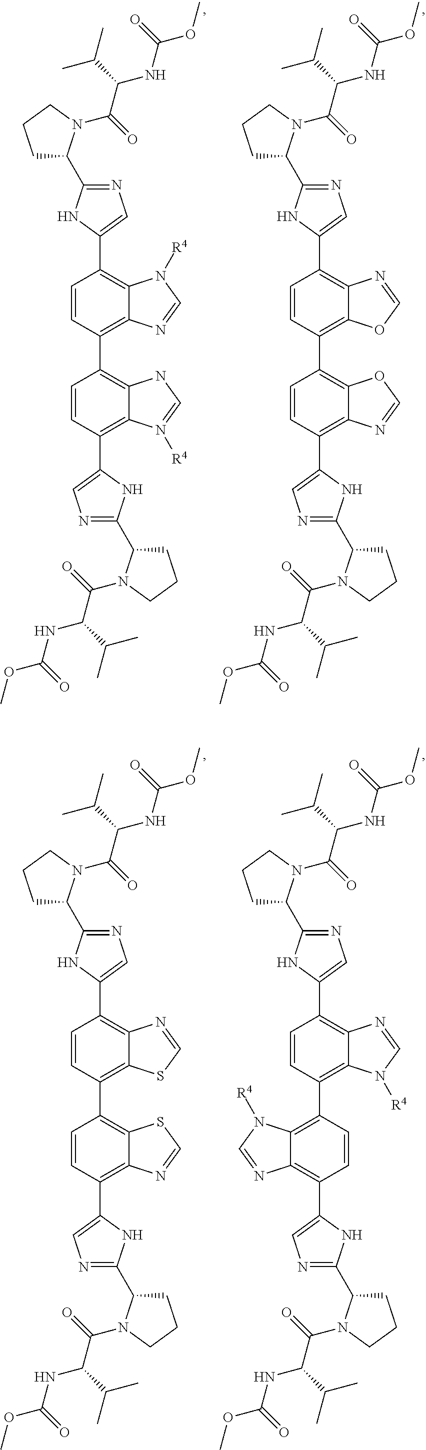 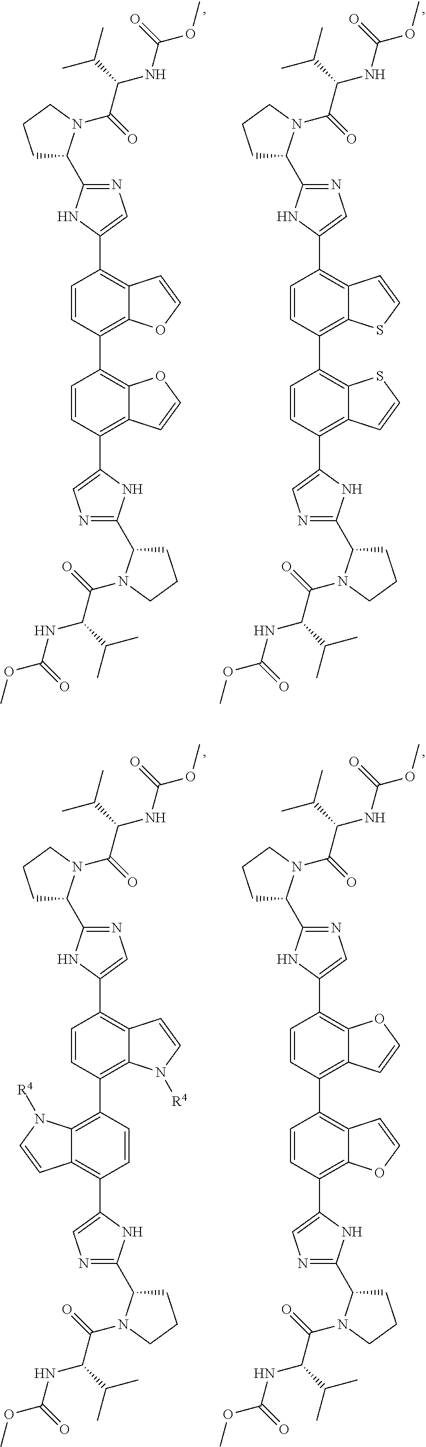 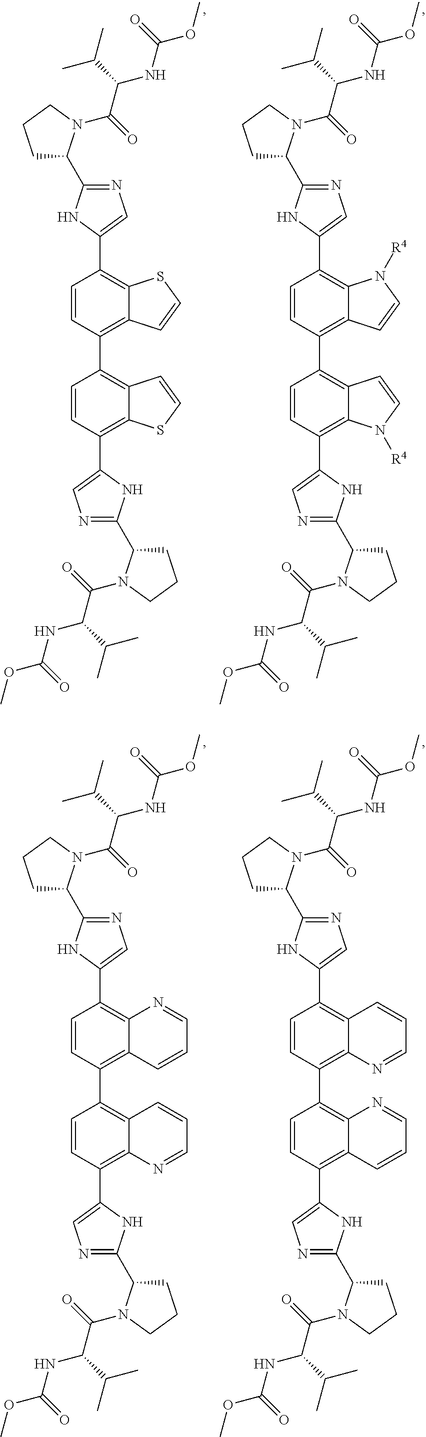 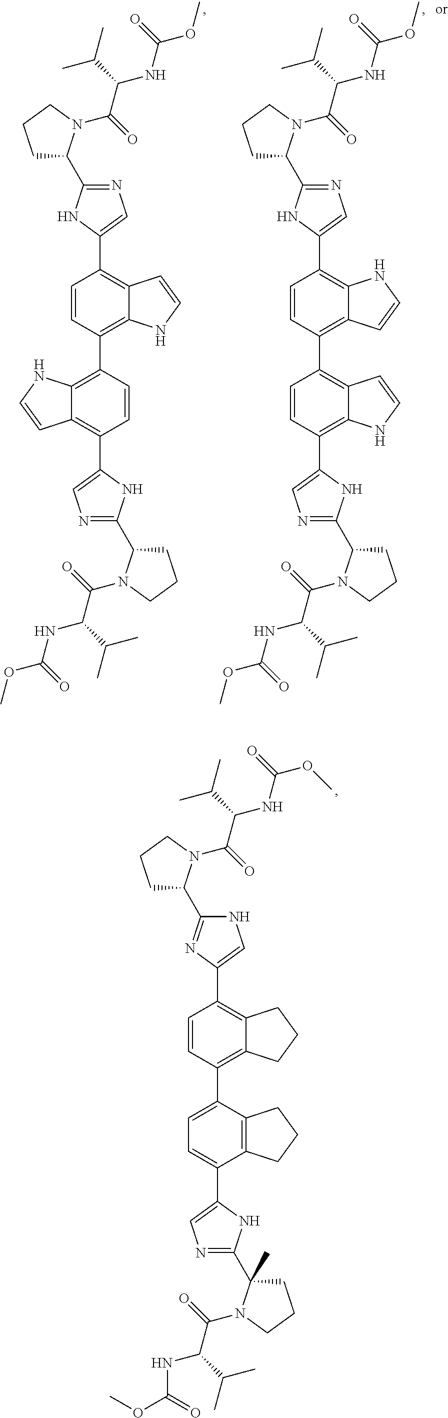
or a pharmaceutically acceptable salt thereof.

18. The compound of claim 1 having the structure of Formula VIIc: 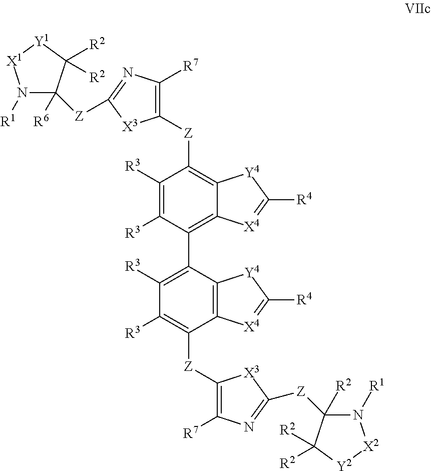
or a pharmaceutically acceptable salt thereof, wherein:
each X⁴ is separately selected from the group consisting of CR⁴ and N (nitrogen); and
each Y⁴ is separately selected from the group consisting of C(R⁴)₂, NR⁴, O (oxygen), and S (sulfur).

19. The compound of claim 1 having the structure of Formula VIId: 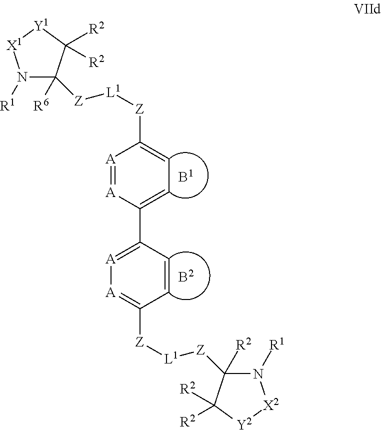
or a pharmaceutically acceptable salt thereof, wherein:
R⁶ is C₁₋₆alkyl optionally substituted with up to 9 halo.

20. The compound of claim 19, wherein R⁶ is methyl.

21. A compound having the structure of Formula VI: 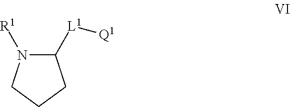
or a pharmaceutically acceptable salt thereof,
wherein:
R¹ is selected from the group consisting of hydrogen, R^{1a}C(═O)— and R^{1a}C(═S)—;
each R^{1a} is separately selected from the group consisting of —C(R^{2a})₂NR^{3a}R^{3b}, alkoxyalkyl, C₁₋₆alkylOC(═O)—, C₁₋₆alkylOC(═O)C₁₋₆alkyl, C₁₋₆alkylC(═O)C₁₋₆alkyl, aryl, aryl(CH═CH)ₘ—, arylalkylO—, arylalkyl, arylOalkyl, cycloalkyl, (cycloalkyl)(CH═CH)ₘ—, (cycloalkyl)alkyl, cycloalkylOalkyl, heterocyclyl, heterocyclyl(CH═CH)ₘ—, heterocyclylalkoxy, heterocyclylalkyl, heterocyclylOalkyl, hydroxyalkyl, R^{c}R^{d}N—, (R^{c}R^{d}N)(CH═CH)ₘ—, (R^{c}R^{d}N)alkyl, (R^{c}R^{d}N)C(═O)—, C₁₋₆alkoxy optionally substituted with up to 9 halo, and C₁₋₆alkyl optionally substituted with up to 9 halo;
each R^{c}R^{d}N is separately selected, wherein R^{c} and R^{d} are each independently selected from hydrogen, alkoxyC(═O)—, C₁₋₆alkyl, C₁₋₆alkylC(═O)—, C₁₋₆alkylsulfonyl, arylalkylOC(═O)—, arylalkyl, arylalkylC(═O)—, arylC(═O)—, arylsulfonyl, heterocyclylalkyl, heterocyclylalkylC(═O)—, heterocyclylC(═O)—, (R^{e}R^{f}N)alkyl, (R^{e}R^{f}N)alkylC(═O)—, and (R^{e}R^{f}N)C(═O)—, wherein the alkyl part of arylalkyl, arylalkylC(═O)—, heterocyclylalkyl, and heterocyclylalkylC(═O)— are each optionally substituted with one R^{e}R^{f}N— group; and wherein the aryl part of arylalkyl, arylalkylC(═O)—, arylC(═O)—, and arylsulfonyl, and the heterocyclyl part of heterocyclylalkyl, heterocyclylalkylC(═O)—, and heterocyclylC(═O)— are each optionally substituted with up to three substituents each independently selected from the group consisting of cyano, halo, nitro, C₁₋₆alkoxy optionally substituted with up to 9 halo, and C₁₋₆alkyl optionally substituted with up to 9 halo;
each R^{e}R^{f}N is separately selected, wherein R^{e} and R^{f} are each separately selected from hydrogen, C₁₋₆alkyl, aryl, arylalkyl, cycloalkyl, (cyclolalkyl)alkyl, heterocyclyl, heterocyclylalkyl, (R^{x}R^{y}N)alkyl, and (R^{x}R^{y}N)C(═O)—;
each R^{x}R^{y}N is separately selected, wherein R^{x} and R^{y} are each separately selected from hydrogen, alkylOC(═O)—, alkyl, alkylC(═O)—, aryl, arylalkyl, cycloalkyl, and heterocyclyl;
each R^{2a} is separately selected from the group consisting of hydrogen, C₁₋₆alkyl, aryl(CH₂)ₙ—, and heteroaryl(CH₂)ₙ—;
each R^{3a} is separately selected from the group consisting of hydrogen, and optionally substituted C₁₋₆alkyl;
each R^{3b} is separately selected from the group consisting of optionally substituted C₁₋₆alkyl, heteroaryl, —(CH₂)ₙC(═O)NR^{4a}R^{4b}, —(CH₂)ₙC(═O)OR^{5a}, and —(CH₂)ₙC(═O)R^{6a} said heteroaryl optionally substituted with cyano, halo, nitro, hydroxyl, C₁₋₆alkoxy optionally substituted with up to 9 halo, and C₁₋₆alkyl optionally substituted with up to 9 halo;
each R^{4a}R^{4b}N is separately selected, wherein R^{4a} and R^{4b} are each separately selected from the group consisting of hydrogen, optionally substituted C₁₋₆alkyl, and aryl(CH₂)ₙ—;
each R^{5a} is separately selected from the group consisting of optionally substituted C₁₋₆alkyl, and aryl(CH₂)ₙ—;
each R^{6a} is separately selected from the group consisting of optionally substituted C₁₋₆alkyl, and aryl(CH₂)ₙ—;
L¹ is selected from the group consisting of 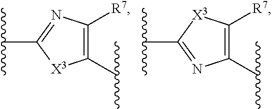
—C(═O)(CH₂)ₘOC(═O)—, —C(CF₃)₂NR^{2c}—, and 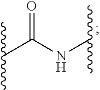
R^{2c} is selected from the group consisting of hydrogen, C₁₋₆alkyl, C₂₋₆alkenyl, C₂₋₆alkynyl, C₃₋₇cycloalkyl, aryl, arylalkyl, heteroaryl, heteroarylalkyl, heterocyclyl, and heterocyclylalkyl, said alkyl optionally substituted with R^{e}R^{f}N—, alkoxy, or C₁₋₆alkylS—;
each X³ is separately selected from the group consisting of NH, NC₁₋₆alkyl, O (oxygen), and S (sulfur);
each m separately is 1 or 2;
each n separately is 0, 1 or 2;
each R⁷ is separately selected from the group consisting of hydrogen, C₁₋₆alkylOC(═O)—, arylalkylOC(═O)—, —COOH, (R^{a}R^{b}N)C(═O)—, trialkylsilylalkylOalkyl, and C₁₋₆alkyl optionally substituted with up to 9 halo;
Q¹ is selected from the group consisting of L² and L³-L⁴.
L² is selected from the group consisting of 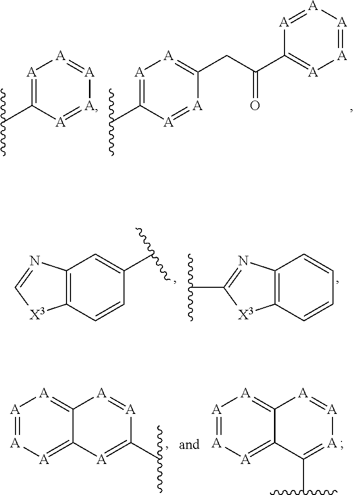
each A is separately selected from the group consisting of CR³ and N (nitrogen);
each R³ is separately selected from the group consisting of hydrogen, C₁₋₆alkoxy, C₁₋₆alkylOC₁₋₆alkyl, C₁₋₆alkylOC(═O)—, arylalkylOC(═O)—, —COOH, halo, hydroxy, R^{a}R^{b}N—, (R^{a}R^{b}N)alkyl, (R^{a}R^{b}N)C(═O)—, and C₁₋₆alkyl optionally substituted with up to 9 halo and up to 5 hydroxy;
each R^{a}R^{b}N is separately selected, wherein R^{a} and R^{b} are each separately selected from the group consisting of hydrogen, C₂₋₆alkenyl, and C₁₋₆alkyl. 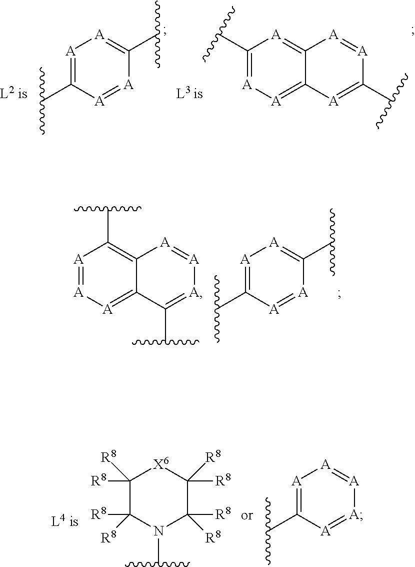
each R⁸ is separately selected from the group consisting of hydrogen, C₁₋₆alkoxy, C₁₋₆alkylOC₁₋₆alkyl, C₁₋₆alkylOC(═O)—, arylalkylOC(═O)—, —COOH, halo, hydroxy, R^{a}R^{b}N—, (R^{a}R^{b}N)alkyl, (R^{a}R^{b}N)C(═O)—, C₁₋₆alkyl optionally substituted with up to 9 halo and C₁₋₆alkyl substituted with up to 5 hydroxy, or optionally two geminal R⁸ are together oxo;
X⁶ is selected from the group consisting of O (oxygen), NR⁹ (nitrogen), and C(R⁸)₂; and
R⁹ is separately selected from the group consisting of hydrogen, aryl(CH₂)ₙ—, C₁₋₆alkylO(CH₂)ₙ, C₁₋₆alkylOC(═O)—, C₁₋₆alkylNHC(═O)—, C₁₋₆alkylC(═O)—, arylC(═O)—, arylOC(═O)—, arylNHC(═O)—, arylalkylOC(═O)—, (R^{a}R^{b}N)(CH₂)ₙ, (R^{a}R^{b}N)C(═O)—, and C₁₋₆alkyl optionally substituted with up to 9 halo, said aryl(CH₂)ₙ—, arylC(═O)—, arylOC(═O)—, and arylNHC(═O)—, each optionally substituted with up to 5 substituents each individually selected from the group consisting of halo, hydroxy, cyano, nitro, C₁₋₆alkyl optionally substituted with up to 9 halo, and C₁₋₆alkoxy optionally substituted with up to 9 halo.

22. The Compound of claim 21, having the structure: 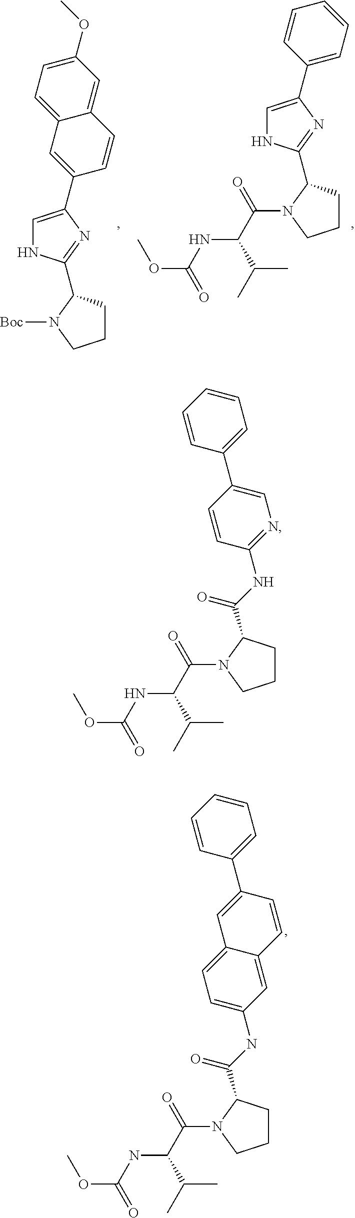 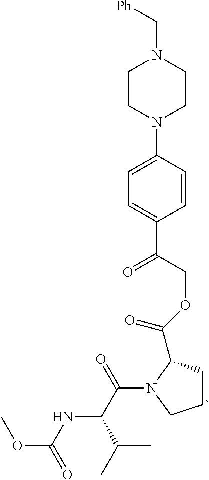
or a pharmaceutically acceptable salt thereof.

23. A compound having the structure of Formula VIII: 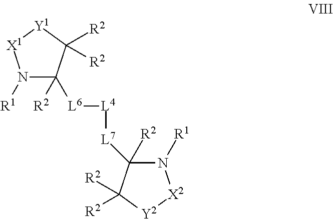
or a pharmaceutically acceptable salt thereof,
wherein:
each R¹ is separately selected from the group consisting of hydrogen, R^{1a}C(═O)— and R^{1a}C(═S)—;
each R^{1a} is separately selected from the group consisting of —C(R^{2a})₂NR^{3a}R^{3b}, alkoxyalkyl, C₁₋₆alkylOC(═O)—, C₁₋₆alkylOC(═O)C₁₋₆alkyl, C₁₋₆alkylC(═O)C₁₋₆alkyl, aryl, aryl(CH₂)ₙ—, aryl(CH₂)ₙO, aryl(CH═CH)ₘ—, arylalkylO—, arylalkyl, arylOalkyl, cycloalkyl, (cycloalkyl)(CH═CH)ₘ—, (cycloalkyl)alkyl, cycloalkylOalkyl, heterocyclyl, heterocyclyl(CH═CH)ₘ—, heterocyclylalkoxy, heterocyclylalkyl, heterocyclylOalkyl, hydroxyalkyl, R^{c}R^{d}N—, R^{c}R^{d}N(CH₂)ₙ—, (R^{c}R^{d}N)(CH═CH)ₘ—, (R^{c}R^{d}N)alkyl, (R^{c}R^{d}N)C(═O)—, C₁₋₆alkoxy optionally substituted with up to 9 halo, and C₁₋₆alkyl optionally substituted with up to 9 halo, said aryl and heteroaryl each optionally substituted with cyano, halo, nitro, hydroxyl, C₁₋₆alkoxy optionally substituted with up to 9 halo, and C₁₋₆alkyl optionally substituted with up to 9 halo;
each R^{c}R^{d}N is separately selected, wherein R^{c} and R^{d} are each separately selected from hydrogen, alkoxyC(═O)—, C₁₋₆alkyl, C₁₋₆alkylC(═O)—, C₁₋₆alkylsulfonyl, arylalkylOC(═O)—, arylalkyl, arylalkylC(═O)—, arylC(═O)—, arylsulfonyl, heterocyclylalkyl, heterocyclylalkylC(═O)—, heterocyclylC(═O)—, (R^{e}R^{f}N)alkyl, (R^{e}R^{f}N)alkylC(═O)—, and (R^{e}R^{f}N)C(═O)—, wherein the alkyl part of arylalkyl, arylalkylC(═O)—, heterocyclylalkyl, and heterocyclylalkylC(═O)— are each optionally substituted with one R^{e}R^{f}N— group; and wherein the aryl part of arylalkyl, arylalkylC(═O)—, arylC(═O)—, and arylsulfonyl, and the heterocyclyl part of heterocyclylalkyl, heterocyclylalkylC(═O)—, and heterocyclylC(═O)— are each optionally substituted with up to three substituents each independently selected from the group consisting of cyano, halo, nitro, C₁₋₆alkoxy optionally substituted with up to 9 halo, and C₁₋₆alkyl optionally substituted with up to 9 halo;
each R^{e}R^{f}N is separately selected, wherein R^{e} and R^{f} are each separately selected from hydrogen, C₁₋₆alkyl, aryl, arylalkyl, cycloalkyl, (cyclolalkyl)alkyl, heterocyclyl, heterocyclylalkyl, (R^{x}R^{y}N)alkyl, and (R^{x}R^{y}N)C(═O)—;
each R^{x}R^{y}N is separately selected, wherein R^{x} and R^{y} are each separately selected from hydrogen, C₁₋₆alkylOC(═O)—, C₁₋₆alkyl, C₁₋₆alkylC(═O)—, aryl, arylalkyl, cycloalkyl, and heterocyclyl;
each C(R^{2a})₂ is separately selected, wherein each R^{2a} is separately selected from the group consisting of hydrogen, C₁₋₆alkyl optionally substituted with up to 9 halo, aryl(CH₂)ₙ—, and heteroaryl(CH₂)ₙ—, said aryl and heteroaryl each optionally substituted with cyano, halo, nitro, hydroxyl, C₁₋₆alkoxy optionally substituted with up to 9 halo, and C₁₋₆alkyl optionally substituted with up to 9 halo, or C(R^{2a})₂ is 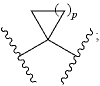
each R^{3a} is separately selected from the group consisting of hydrogen, and optionally substituted C₁₋₆alkyl;
each R^{3b} is separately selected from the group consisting of optionally substituted C₁₋₆alkyl, heteroaryl, —(CH₂)ₙC(═O)NR^{4a}R^{4b}, —(CH₂)ₙC(═O)OR^{5a}, and —(CH₂)ₙC(═O)R^{6a} said heteroaryl optionally substituted with cyano, halo, nitro, hydroxyl, C₁₋₆alkoxy optionally substituted with up to 9 halo, and C₁₋₆alkyl optionally substituted with up to 9 halo;
each R^{4a}R^{4b}N is separately selected, wherein R^{4a} and R^{4b} are each separately selected from the group consisting of hydrogen, optionally substituted C₁₋₆alkyl, and aryl(CH₂)ₙ—;
each R^{5a} is separately selected from the group consisting of optionally substituted C₁₋₆alkyl, and aryl(CH₂)ₙ—;
each R^{6a} is separately selected from the group consisting of optionally substituted C₁₋₆alkyl, and aryl(CH₂)ₙ—; 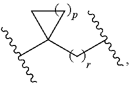
X¹ is (C(R²)₂)_{q}, or X¹ is null;
Y¹ is selected from O (oxygen), S (sulfur), S(O), SO₂, NR², and C(R²)₂ with the proviso that when X¹ is null Y¹ is C(R²)₂;
X² is (C(R²)₂)_{q}, 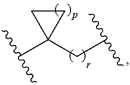
or X² is null;
Y² is selected from O (oxygen), S (sulfur), S(O), SO₂, NR², and C(R²)₂ with the proviso that when X² is null Y² is C(R²)₂;
each R² is separately selected, wherein R² is selected from the group consisting of hydrogen, C₁₋₆alkoxy, C₁₋₆alkyl, aryl, halo, hydroxy, R^{a}R^{b}N—, and C₁₋₆alkyl optionally substituted with up to 9 halo, or optionally two vicinal R² and the carbons to which they are attached are together a fused three- to six-membered carbocyclic ring optionally substituted with up to two C₁₋₆alkyl groups;
each R^{a}R^{b}N is separately selected, wherein R^{a} and R^{b} are each separately selected from the group consisting of hydrogen, C₂₋₆alkenyl, and C₁₋₆alkyl;
L⁴ is selected from the group consisting of 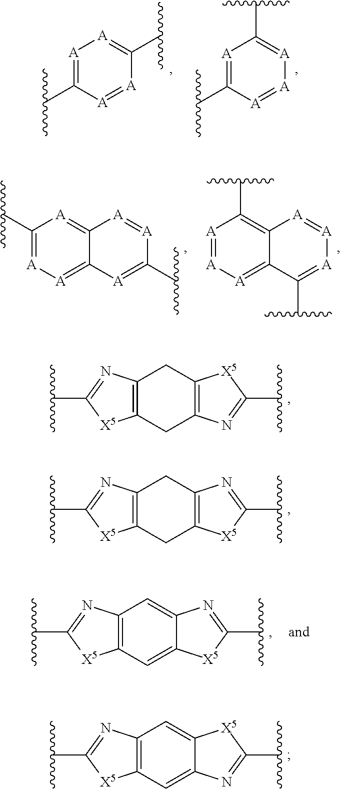
each X⁵ is separately selected from the group consisting of —NH—, O (oxygen), S (sulfur), and —CH₂—;
each A is separately selected from the group consisting of CR³ and N (nitrogen);
each R³ is separately selected from the group consisting of hydrogen, C₁₋₆alkoxy, C₁₋₆alkylOC₁₋₆alkyl, C₁₋₆alkylOC(═O)—, arylalkylOC(═O)—, —COOH, halo, hydroxy, R^{a}R^{b}N—, (R^{a}R^{b}N)alkyl, (R^{a}R^{b}N)C(═O)—, C₁₋₆alkyl optionally substituted with up to 9 halo and C₁₋₆alkyl substituted with up to 5 hydroxy;
L⁶ is selected from the group consisting of 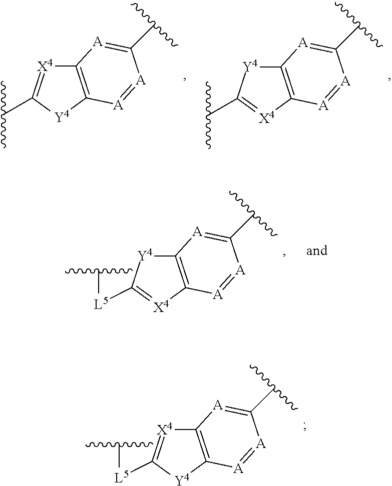
L⁷ is selected from the group consisting of 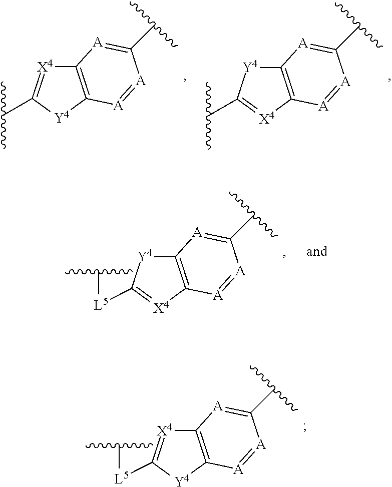
each L⁵ is separately selected from the group consisting of 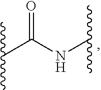 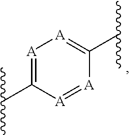
and —(CH═CH)—;
each X⁴ is separately selected from the group consisting of CR⁴ and N (nitrogen);
each Y⁴ is separately selected from the group consisting of C(R⁴)₂, NR⁴, O (oxygen), and S (sulfur);
each R⁴ is separately selected from the group consisting of C₁₋₆alkoxy, C₁₋₆alkylOC₁₋₆alkyl, C₁₋₆alkylOC(═O)—, arylalkylOC(═O)—, —COOH, halo, C₁₋₆haloalkyl, hydroxy, R^{a}R^{b}N—, (R^{a}R^{b}N)alkyl, (R^{a}R^{b}N)C(═O)—, C₁₋₆alkyl optionally substituted with up to 9 halo and C₁₋₆alkyl substituted with up to 5 hydroxy, or optionally two geminal R⁴ are together oxo;
each m separately is 1 or 2;
each n separately is 0, 1 or 2;
each p separately is 1, 2, 3 or 4;
each q separately is 1, 2, 3, 4 or 5; and
each r separately is 0, 1, 2, 3, or 4,
with the proviso that L⁶-L⁴-L⁷ is not 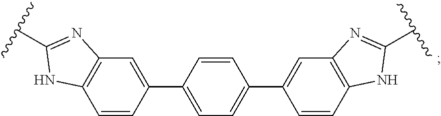
and
that the compound is not selected from the group consisting of:

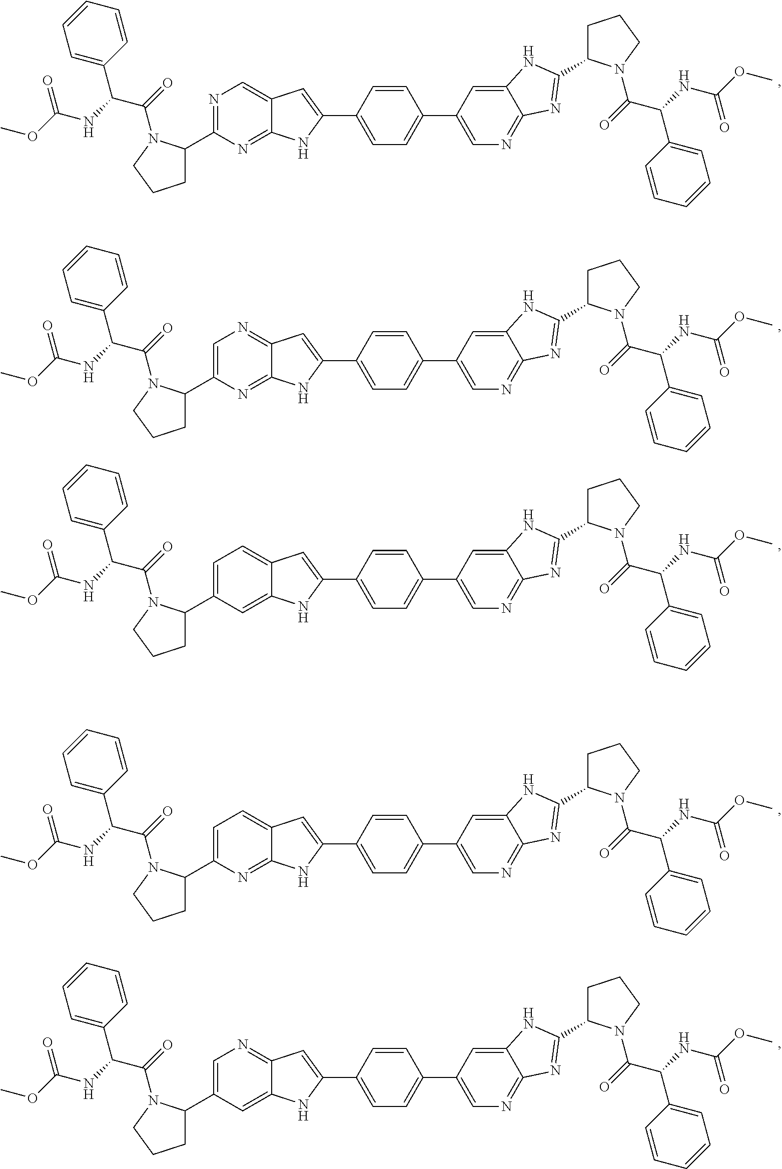

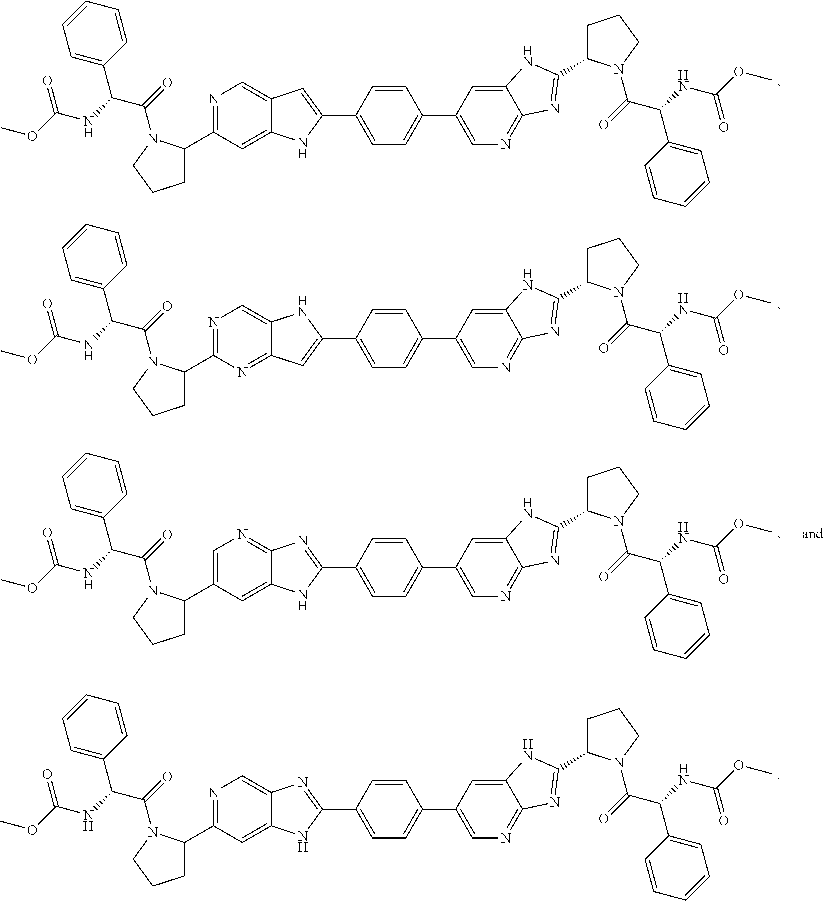

24. The compound of claim 23, wherein each R¹ is R^{1a}C(═O)—.

25. The compound of claim 24, wherein each R^{1a} is —CHR^{2a}NHR^{3b}.

26. The compound of claim 23, wherein each R^{2a} is C₁₋₆alkyl;
each R^{3b} is —C(═O)OR⁵; and
each R⁵ is C₁₋₆alkyl.

27. The compound of claim 23,
wherein:
L⁶-L⁴-L⁷ is selected from the group consisting of

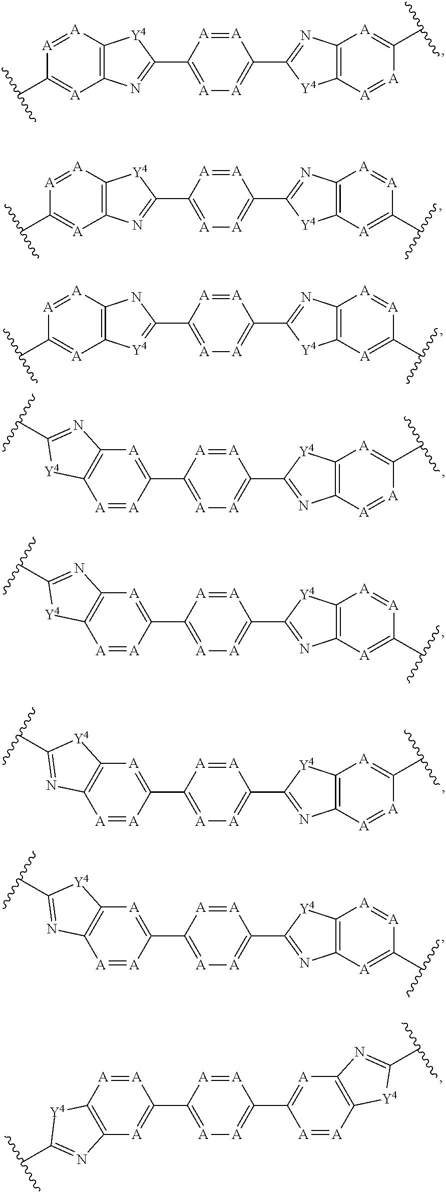

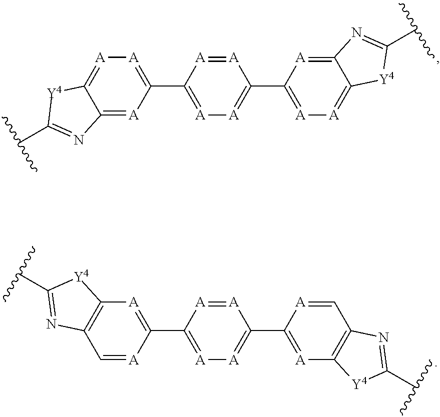

28. The compound of claim 23,
wherein:
L⁶ is selected from the group consisting of 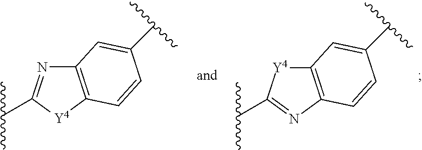
L⁷ is selected from the group consisting of

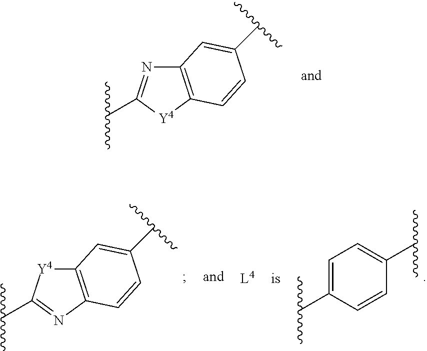

29. The compound of claim 23 having the structure of Formula VIIIa: 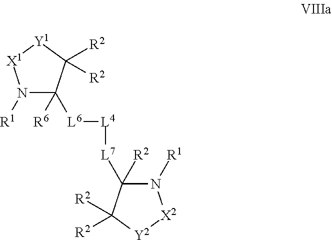
or a pharmaceutically acceptable salt thereof, wherein R⁶ is C₁₋₆alkyl optionally substituted with up to 9 halo.

30. The compound of claim 29, wherein R⁶ is methyl.

31. The compound of claim 23 having the structure: 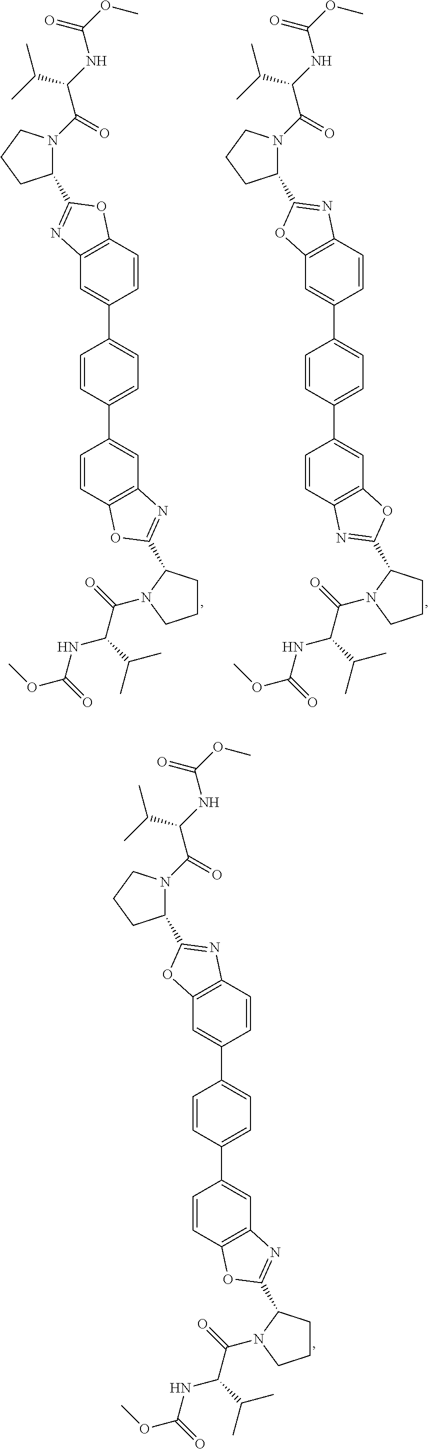 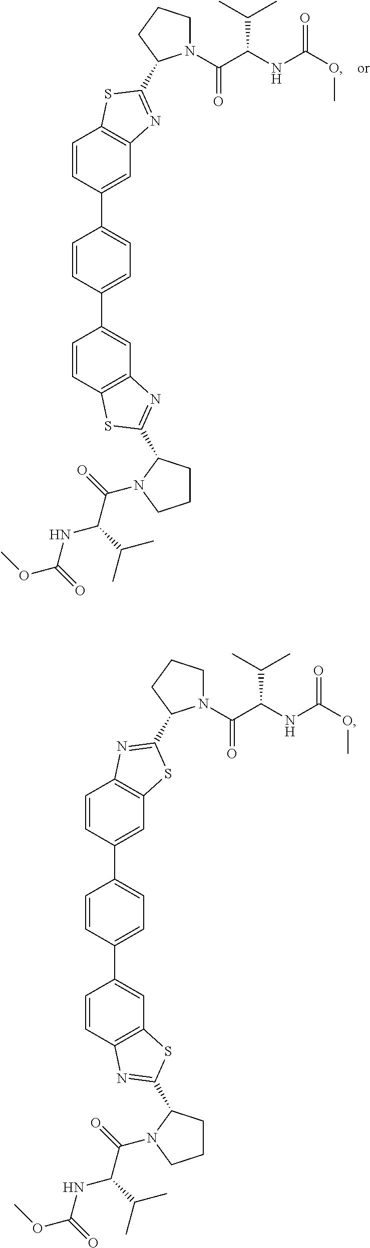
or a pharmaceutically acceptable salt thereof.

32. A compound having the structure of Formula IX: 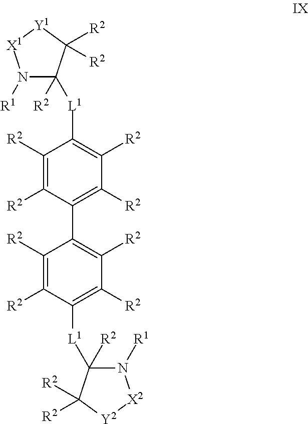
or a pharmaceutically acceptable salt thereof,
wherein:
each R¹ is separately selected from the group consisting of hydrogen, R^{1a}C(═O)— and R^{1a}C(═S)—;
each R^{1a} is separately selected from the group consisting of —C(R^{2a})₂NR^{3a}R^{3b}, alkoxyalkyl, C₁₋₆alkylOC(═O)—, C₁₋₆alkylOC(═O)C₁₋₆alkyl, C₁₋₆alkylC(═O)C₁₋₆alkyl, aryl, aryl(CH₂)ₙ—, aryl(CH₂)ₙO—, aryl(CH═CH)ₘ—, arylalkylO—, arylalkyl, arylOalkyl, cycloalkyl, (cycloalkyl)(CH═CH)ₘ—, (cycloalkyl)alkyl, cycloalkylOalkyl, heterocyclyl, heterocyclyl(CH═CH)ₘ—, heterocyclylalkoxy, heterocyclylalkyl, heterocyclylOalkyl, hydroxyalkyl, R^{c}R^{d}N—, R^{c}R^{d}N(CH₂)ₙ—, (R^{c}R^{d}N)(CH═CH)ₘ—, (R^{c}R^{d}N)alkyl, (R^{c}R^{d}N)C(═O)—, C₁₋₆alkoxy optionally substituted with up to 9 halo, and C₁₋₆alkyl optionally substituted with up to 9 halo, said aryl and heteroaryl each optionally substituted with cyano, halo, nitro, hydroxyl, C₁₋₆alkoxy optionally substituted with up to 9 halo, and C₁₋₆alkyl optionally substituted with up to 9 halo;
each R^{c}R^{d}N is separately selected, wherein R^{c} and R^{d} are each separately selected from the group consisting of hydrogen, alkoxyC(═O)—, C₁₋₆alkyl, C₁₋₆alkylC(═O)—, C₁₋₆alkylsulfonyl, arylalkylOC(═O)—, arylalkyl, arylalkylC(═O)—, arylC(═O)—, arylsulfonyl, heterocyclylalkyl, heterocyclylalkylC(═O)—, heterocyclylC(═O)—, (R^{e}R^{f}N)alkyl, (R^{e}R^{f}N)alkylC(═O)—, and (R^{e}R^{f}N)C(═O)—, wherein the alkyl part of arylalkyl, arylalkylC(═O)—, heterocyclylalkyl, and heterocyclylalkylC(═O)— are each optionally substituted with one R^{e}R^{f}N— group; and wherein the aryl part of arylalkyl, arylalkylC(═O)—, arylC(═O)—, and arylsulfonyl, and the heterocyclyl part of heterocyclylalkyl, heterocyclylalkylC(═O)—, and heterocyclylC(═O)— are each optionally substituted with up to three substituents each independently selected from the group consisting of cyano, halo, nitro, C₁₋₆alkoxy optionally substituted with up to 9 halo, and C₁₋₆alkyl optionally substituted with up to 9 halo;
each R^{e}R^{f}N is separately selected, wherein R^{e} and R^{f} are each separately selected from the group consisting of hydrogen, C₁₋₆alkyl, aryl, arylalkyl, cycloalkyl, (cyclolalkyl)alkyl, heterocyclyl, heterocyclylalkyl, (R^{x}R^{y}N)alkyl, and (R^{x}R^{y}N)C(═O)—;
each R^{X}R^{y}N is separately selected, wherein R^{x} and R^{y} are each separately selected from the group consisting of hydrogen, C₁₋₆alkylOC(═O)—, C₁₋₆alkyl, C₁₋₆alkylC(═O)—, aryl, arylalkyl, cycloalkyl, and heterocyclyl;
each C(R^{2a})₂ is separately selected, wherein each R^{2a} is separately selected from the group consisting of hydrogen, C₁₋₆alkyl optionally substituted with up to 9 halo, aryl(CH₂)ₙ—, and heteroaryl(CH₂)ₙ—, said aryl and heteroaryl each optionally substituted with cyano, halo, nitro, hydroxyl, C₁₋₆alkoxy optionally substituted with up to 9 halo, and C₁₋₆alkyl optionally substituted with up to 9 halo, or C(R^{2a})₂ is 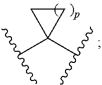
each R^{3a} is separately selected from the group consisting of hydrogen, and optionally substituted C₁₋₆alkyl;
each R^{3b} is separately selected from the group consisting of optionally substituted C₁₋₆alkyl, heteroaryl, —(CH₂)ₙC(═O)NR^{4a}R^{4b}, —(CH₂)ₙC(═O)OR^{5a}, and —(CH₂)ₙC(═O)R^{6a} said heteroaryl optionally substituted with cyano, halo, nitro, hydroxyl, C₁₋₆alkoxy optionally substituted with up to 9 halo, and C₁₋₆alkyl optionally substituted with up to 9 halo;
each R^{4a}R^{4b}N is separately selected, wherein R^{4a} and R^{4b} are each separately selected from the group consisting of hydrogen, optionally substituted C₁₋₆alkyl, and aryl(CH₂)ₙ—;
each R^{5a} is separately selected from the group consisting of C₁₋₆alkyl optionally substituted with up to five R² groups, and aryl(CH₂)ₙ—;
each R^{6a} is separately selected from the group consisting of optionally substituted C₁₋₆alkyl, and aryl(CH₂)ₙ—;
X¹ is (C(R²)₂)_{q}, 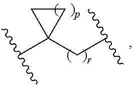
or X¹ is null;
Y¹ is selected from O (oxygen), S (sulfur), S(O), SO₂, NR², and C(R²)₂ with the proviso that when X¹ is null Y¹ is C(R²)₂;
X² is (C(R²)₂)_{q}, 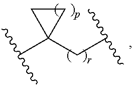
or X² is null;
Y² is selected from O (oxygen), S (sulfur), S(O), SO₂, NR², and C(R²)₂ with the proviso that when X² is null Y² is C(R²)₂;
each R² is separately selected, wherein R² is selected from the group consisting of hydrogen, deuterium, C₁₋₆alkoxy, C₁₋₆alkyl, aryl, halo, hydroxy, R^{a}R^{b}N—, and C₁₋₆alkyl optionally substituted with up to 9 halo, or optionally two R² and the carbons to which they are attached are together a fused three- to eight-membered carbocyclic ring optionally containing one or two heteroatoms each independently selected from O (oxygen), N (nitrogen), and S (sulfur); wherein the three- to eight-membered carbocyclic ring is optionally substituted with one or more substituents selected from the group consisting of deuterium, halo, hydroxyl, oxo, R^{x}R^{y}N, R^{x}R^{y}NC(═O), R^{x}R^{y}NC₁₋₆alkyl, heteroaryl, aryl, C₁₋₆alkyl optionally substituted with up to 9 halo, and C₁₋₆alkoxy optionally substituted with up to 9 halo, wherein at least one R² is deuterium;
each R^{a}R^{b}N is separately selected, wherein R^{a} and R^{b} are each separately selected from the group consisting of hydrogen, C₂₋₆alkenyl, and C₁₋₆alkyl;
each L¹ is separately selected from the group consisting of 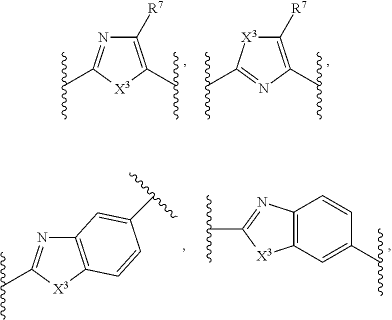
—C(═O)(CH₂)ₘOC(═O)—, —C(CF₃)₂NR^{2c}—, and 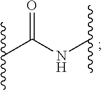
R^{2c} is selected from the group consisting of hydrogen, C₁₋₆alkyl, C₂₋₆alkenyl, C₂₋₆alkynyl, C₃₋₇cycloalkyl, aryl, arylalkyl, heteroaryl, heteroarylalkyl, heterocyclyl, and heterocyclylalkyl, said alkyl optionally substituted with R^{e}R^{f}N—, alkoxy, or C₁₋₆alkylS—;
each X³ is separately selected from the group consisting of NH, NC₁₋₆alkyl, O (oxygen), and S (sulfur);
each m separately is 1 or 2;
each n separately is 0, 1 or 2;
each p separately is 1, 2, 3 or 4;
each q separately is 1, 2, 3, 4 or 5;
each r separately is 0, 1, 2, 3, or 4; and
each R⁷ is separately selected from the group consisting of hydrogen, C₁₋₆alkylOC(═O)—, arylalkylOC(═O)—, —COOH, (R^{a}R^{b}N)C(═O)—, trialkylsilylalkylOalkyl, and C₁₋₆alkyl optionally substituted with up to 9 halo.

33. The compound of claim 32, wherein each R¹ is R^{1a}C(═O)—.

34. The compound of claim 32, wherein each R^{1a} is —CHR^{2a}NHR^{3b}.

35. The compound of claim 32, wherein each R^{2a} is C₁₋₆alkyl;
each R^{3b} is —C(═O)OR⁵; and
each R⁵ is C₁₋₆alkyl.

36. The compound of claim 32 having the structure of Formula IXa: 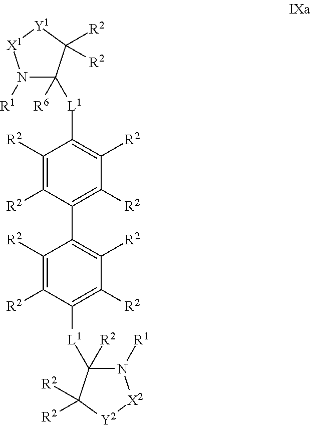
or a pharmaceutically acceptable salt thereof, wherein R⁶ is C₁₋₆alkyl optionally substituted with up to 9 halo.

37. The compound of claim 36, wherein R⁶ is methyl.

38. A compound having the structure of Formula IX: 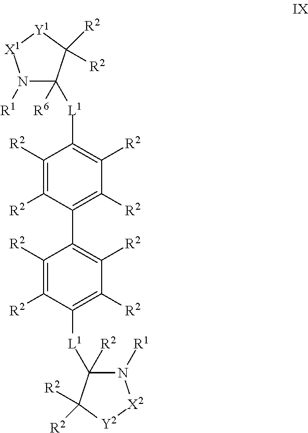
or a pharmaceutically acceptable salt thereof,
wherein:
each R¹ is separately selected from the group consisting of hydrogen, R^{1a}C(═O)— and R^{1a}C(═S)—;
each R^{1a} is separately selected from the group consisting of —C(R^{2a})₂NR^{3a}R^{3b}, alkoxyalkyl, C₁₋₆alkylOC(═O)—, C₁₋₆alkylOC(═O)C₁₋₆alkyl, C₁₋₆alkylC(═O)C₁₋₆alkyl, aryl, aryl(CH₂)ₙ—, aryl(CH₂)ₙO—, aryl(CH═CH)ₘ—, arylalkylO—, arylalkyl, arylOalkyl, cycloalkyl, (cycloalkyl)(CH═CH)ₘ—, (cycloalkyl)alkyl, cycloalkylOalkyl, heterocyclyl, heterocyclyl(CH═CH)ₘ—, heterocyclylalkoxy, heterocyclylalkyl, heterocyclylOalkyl, hydroxyalkyl, R^{c}R^{d}N—, R^{c}R^{d}N(CH₂)ₙ—, (R^{c}R^{d}N)(CH═CH)ₘ—, (R^{c}R^{d}N)alkyl, (R^{c}R^{d}N)C(═O)—, C₁₋₆alkoxy optionally substituted with up to 9 halo, and C₁₋₆alkyl optionally substituted with up to 9 halo, said aryl and heteroaryl each optionally substituted with cyano, halo, nitro, hydroxyl, C₁₋₆alkoxy optionally substituted with up to 9 halo, and C₁₋₆alkyl optionally substituted with up to 9 halo;
each R^{c}R^{d}N is separately selected, wherein R^{c} and R^{d} are each separately selected from hydrogen, alkoxyC(═O)—, C₁₋₆alkyl, C₁₋₆alkylC(═O)—, C₁₋₆alkylsulfonyl, arylalkylOC(═O)—, arylalkyl, arylalkylC(═O)—, arylC(═O)—, arylsulfonyl, heterocyclylalkyl, heterocyclylalkylC(═O)—, heterocyclylC(═O)—, (R^{e}R^{f}N)alkyl, (R^{e}R^{f}N)alkylC(═O)—, and (R^{e}R^{f}N)C(═O)—, wherein the alkyl part of arylalkyl, arylalkylC(═O)—, heterocyclylalkyl, and heterocyclylalkylC(═O)— are each optionally substituted with one R^{e}R^{f}N— group; and wherein the aryl part of arylalkyl, arylalkylC(═O)—, arylC(═O)—, and arylsulfonyl, and the heterocyclyl part of heterocyclylalkyl, heterocyclylalkylC(═O)—, and heterocyclylC(═O)— are each optionally substituted with up to three substituents each independently selected from the group consisting of cyano, halo, nitro, C₁₋₆alkoxy optionally substituted with up to 9 halo, and C₁₋₆alkyl optionally substituted with up to 9 halo;
each R^{e}R^{f}N is separately selected, wherein R^{e} and R^{f} are each separately selected from hydrogen, C₁₋₆alkyl, aryl, arylalkyl, cycloalkyl, (cyclolalkyl)alkyl, heterocyclyl, heterocyclylalkyl, (R^{x}R^{y}N)alkyl, and (R^{x}R^{y}N)C(═O)—;
each R^{x}R^{y}N is separately selected, wherein R^{x} and R^{y} are each separately selected from hydrogen, C₁₋₆alkylOC(═O)—, C₁₋₆alkyl, C₁₋₆alkylC(═O)—, aryl, arylalkyl, cycloalkyl, and heterocyclyl;
each C(R^{2a})₂ is separately selected, wherein each R^{2a} is separately selected from the group consisting of hydrogen, optionally substituted C₁₋₆alkyl, aryl(CH₂)ₙ—, and heteroaryl(CH₂)ₙ—, said aryl and heteroaryl each optionally substituted with cyano, halo, nitro, hydroxyl, C₁₋₆alkoxy optionally substituted with up to 9 halo, and C₁₋₆alkyl optionally substituted with up to 9 halo, or C(R^{2a})₂ is 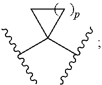
each R^{3a} is separately selected from the group consisting of hydrogen, and optionally substituted C₁₋₆alkyl;
each R^{3b} is separately selected from the group consisting of optionally substituted C₁₋₆alkyl, heteroaryl, —(CH₂)ₙC(═O)NR^{4a}R^{4b}, —(CH₂)ₙC(═O)OR^{5a}, and —(CH₂)ₙC(═O)R^{6a} said heteroaryl optionally substituted with cyano, halo, nitro, hydroxyl, C₁₋₆alkoxy optionally substituted with up to 9 halo, and C₁₋₆alkyl optionally substituted with up to 9 halo;
each R^{4a}R^{4b}N is separately selected, wherein R^{4a} and R^{4b} are each separately selected from the group consisting of hydrogen, optionally substituted C₁₋₆alkyl, and aryl(CH₂)ₙ—;
each R^{5a} is separately selected from the group consisting of optionally substituted C₁₋₆alkyl and aryl(CH₂)ₙ—;
each R^{6a} is separately selected from the group consisting of optionally substituted C₁₋₆alkyl, and aryl(CH₂)ₙ—;
X¹ is (C(R²)₂)_{q}, 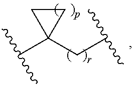
or X¹ is null;
Y¹ is selected from O (oxygen), S (sulfur), S(O), SO₂, NR², and C(R²)₂ with the proviso that when X¹ is null Y¹ is C(R²)₂;
X² is (C(R²)₂)_{q}, 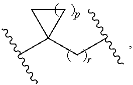
or X² is null;
Y² is selected from O (oxygen), S (sulfur), S(O), SO₂, NR², and C(R²)₂ with the proviso that when X² is null Y² is C(R²)₂;
each R² is separately selected, wherein R² is selected from the group consisting of hydrogen, C₁₋₆alkoxy, aryl, halo, hydroxy, R^{a}R^{b}N—, and C₁₋₆alkyl optionally substituted with up to 9 halo, or optionally two R² and the carbons to which they are attached are together a fused three- to eight-membered carbocyclic ring optionally containing one or two heteroatoms each independently selected from O (oxygen), N (nitrogen), and S (sulfur); wherein the three- to eight-membered carbocyclic ring is optionally substituted with one or more substituents selected from the group consisting of halo, hydroxyl, oxo, R^{x}R^{y}N, R^{x}R^{y}NC(═O), R^{x}R^{y}NC₁₋₆alkyl, heteroaryl, aryl, C₁₋₆alkyl optionally substituted with up to 9 halo, and C₁₋₆alkoxy optionally substituted with up to 9 halo;
each R^{a}R^{b}N is separately selected, wherein R^{a} and R^{b} are each separately selected from the group consisting of hydrogen, C₂₋₆alkenyl, and C₁₋₆alkyl;
each L¹ is separately selected from the group consisting of 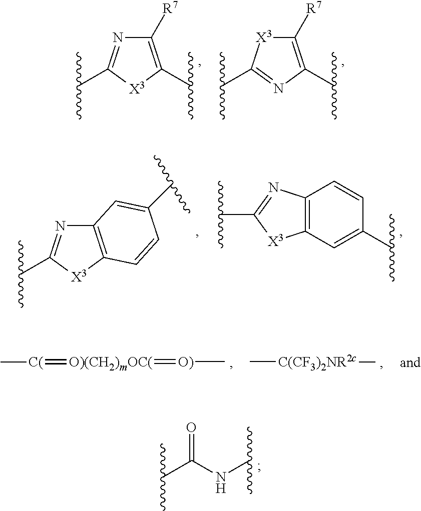
R^{2c} is selected from the group consisting of hydrogen, C₁₋₆alkyl, C₂₋₆alkenyl, C₂₋₆alkynyl, C₃₋₇cycloalkyl, aryl, arylalkyl, heteroaryl, heteroarylalkyl, heterocyclyl, and heterocyclylalkyl, said alkyl optionally substituted with R^{e}R^{f}N—, alkoxy, or C₁₋₆alkylS—;
each X³ is separately selected from the group consisting of NH, NC₁₋₆alkyl, O (oxygen), and S (sulfur);
each m separately is 1 or 2;
each n separately is 0, 1 or 2;
each p separately is 1, 2, 3 or 4;
each q separately is 1, 2, 3, 4 or 5;
each r separately is 0, 1, 2, 3, or 4; and
each R⁷ is separately selected from the group consisting of hydrogen, halo, C₁₋₆alkylOC(═O)—, arylalkylOC(═O)—, —COOH, (R^{a}R^{b}N)C(═O)—, trialkylsilylalkylOalkyl, and C₁₋₆alkyl optionally substituted with up to 9 halo;
wherein at least one of R^{2a}, R^{3a}, R^{4a}, R^{4b}, R^{5a}, and R^{6a} is a substituted C₁₋₆alkyl substituted with at least one substituent that is not alkyl or substituted aryl substituted with at least one substituent that is not alkyl.

39. The compound of claim 38, wherein each R¹ is R^{1a}C(═O)—.

40. The compound of claim 38, wherein each R^{1a} is —CHR^{2a}NHR^{3b}.

41. The compound of claim 38, wherein each R^{2a} is optionally substituted C₁₋₆alkyl;
each R^{3b} is —C(═O)OR^{5a}; and
each R^{5a} is optionally substituted C₁₋₆alkyl.

42. The compound of claim 38, wherein at least one R^{1a} is —C(R^{2a})₂NR^{3a}R^{3b} and within said at least one R^{1a}, at least one R^{2a} is a substituted C₁₋₆alkyl substituted with at least one substituent that is not alkyl or substituted aryl substituted with at least one substituent that is not alkyl.

43. The compound of claim 38, wherein at least one R^{1a} is —C(R^{2a})₂NR^{3a}R^{3b} wherein R^{3b} is —(CH₂)ₙC(═O)OR^{5a} and R^{5a} is a substituted C₁₋₆alkyl substituted with at least one substituent that is not alkyl or substituted aryl substituted with at least one substituent that is not alkyl.

44. The compound of claim 38, wherein at least one of R^{2a}, R^{3a}, R^{4a}, R^{4b}, R^{5a}, and R^{6a} is C₁₋₆alkyl substituted with up to 9 halogen or aryl substituted with up to 9 halogen.

45. The compound of claim 38, wherein each R² is separately selected from the group consisting of hydrogen, C₁₋₆alkoxy, aryl, halo, hydroxy, R^{a}R^{b}N—, and C₁₋₆alkyl optionally substituted with up to 9 halo.

46. The compound of claim 38, wherein at least one of R^{2a}, R^{3a}, R^{4a}, R^{4b}, and R^{6a} is C₁₋₆alkyl substituted with up to 9 halogen or aryl substituted with up to 9 halogen.

47. The compound of claim 38 having the structure of Formula IXa: 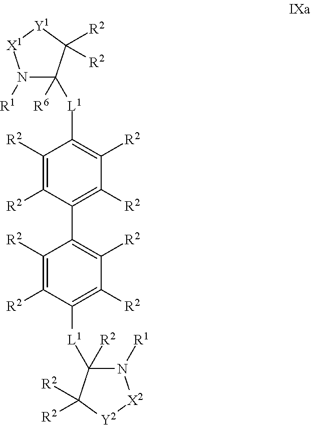
or a pharmaceutically acceptable salt thereof, wherein R⁶ is C₁₋₆alkyl optionally substituted with up to 9 halo.

48. The compound of claim 47, wherein R⁶ is methyl.

49. A compound having the structure of Formula X: 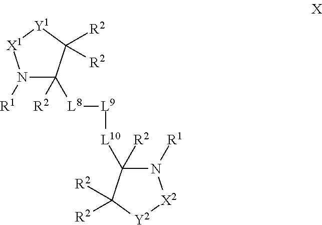
or a pharmaceutically acceptable salt thereof,
wherein:
each R¹ is separately selected from the group consisting of hydrogen, R^{1a}C(═O)— and R^{1a}C(═S)—;
each R^{1a} is separately selected from the group consisting of —C(R^{2a})₂NR^{3a}R^{3b}, alkoxyalkyl, C₁₋₆alkylOC(═O)—, C₁₋₆alkylOC(═O)C₁₋₆alkyl, C₁₋₆alkylC(═O)C₁₋₆alkyl, aryl, aryl(CH₂)ₙ—, aryl(CH₂)ₙO—, aryl(CH═CH)ₘ—, arylalkylO—, arylalkyl, arylOalkyl, cycloalkyl, (cycloalkyl)(CH═CH)ₘ—, (cycloalkyl)alkyl, cycloalkylOalkyl, heterocyclyl, heterocyclyl(CH═CH)ₘ—, heterocyclylalkoxy, heterocyclylalkyl, heterocyclylOalkyl, hydroxyalkyl, R^{c}R^{d}N—, R^{c}R^{d}N(CH₂)ₙ—, (R^{c}R^{d}N)(CH═CH)ₘ—, (R^{c}R^{d}N)alkyl, (R^{c}R^{d}N)C(═O)—, C₁₋₆alkoxy optionally substituted with up to 9 halo, and C₁₋₆alkyl optionally substituted with up to 9 halo, said aryl and heteroaryl each optionally substituted with cyano, halo, nitro, hydroxyl, C₁₋₆alkoxy optionally substituted with up to 9 halo, and C₁₋₆alkyl optionally substituted with up to 9 halo;
each R^{c}R^{d}N is separately selected, wherein R^{c} and R^{d} are each separately selected from hydrogen, alkoxyC(═O)—, C₁₋₆alkyl, C₁₋₆alkylC(═O)—, C₁₋₆alkylsulfonyl, arylalkylOC(═O)—, arylalkyl, arylalkylC(═O)—, arylC(═O)—, arylsulfonyl, heterocyclylalkyl, heterocyclylalkylC(═O)—, heterocyclylC(═O)—, (R^{e}R^{f}N)alkyl, (R^{e}R^{f}N)alkylC(═O)—, and (R^{e}R^{f}N)C(═O)—, wherein the alkyl part of arylalkyl, arylalkylC(═O)—, heterocyclylalkyl, and heterocyclylalkylC(═O)— are each optionally substituted with one R^{e}R^{f}N— group; and wherein the aryl part of arylalkyl, arylalkylC(═O)—, arylC(═O)—, and arylsulfonyl, and the heterocyclyl part of heterocyclylalkyl, heterocyclylalkylC(═O)—, and heterocyclylC(═O)— are each optionally substituted with up to three substituents each independently selected from the group consisting of cyano, halo, nitro, C₁₋₆alkoxy optionally substituted with up to 9 halo, and C₁₋₆alkyl optionally substituted with up to 9 halo;
each R^{e}R^{f}N is separately selected, wherein R^{e} and R^{f} are each separately selected from hydrogen, C₁₋₆alkyl, aryl, arylalkyl, cycloalkyl, (cyclolalkyl)alkyl, heterocyclyl, heterocyclylalkyl, (R^{x}R^{y}N)alkyl, and (R^{x}R^{y}N)C(═O)—;
each R^{x}R^{y}N is separately selected, wherein R^{x} and R^{y} are each separately selected from hydrogen, C₁₋₆alkylOC(═O)—, C₁₋₆alkyl, C₁₋₆alkylC(═O)—, aryl, arylalkyl, cycloalkyl, and heterocyclyl;
each C(R^{2a})₂ is separately selected, wherein each R^{2a} is separately selected from the group consisting of hydrogen, optionally substituted C₁₋₆alkyl, aryl(CH₂)ₙ—, and heteroaryl(CH₂)ₙ—, said aryl and heteroaryl each optionally substituted with cyano, halo, nitro, hydroxyl, C₁₋₆alkoxy optionally substituted with up to 9 halo, and C₁₋₆alkyl optionally substituted with up to 9 halo, or C(R^{2a})₂ is 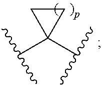
each R^{3a} is separately selected from the group consisting of hydrogen, and optionally substituted C₁₋₆alkyl;
each R^{3b} is separately selected from the group consisting of optionally substituted C₁₋₆alkyl, heteroaryl, —(CH₂)ₙC(═O)NR^{4a}R^{4b}, —(CH₂)ₙC(═O)OR^{5a}, and —(CH₂)ₙC(═O)R^{6a} said heteroaryl optionally substituted with cyano, halo, nitro, hydroxyl, C₁₋₆alkoxy optionally substituted with up to 9 halo, and C₁₋₆alkyl optionally substituted with up to 9 halo;
each R^{4a}R^{4b}N is separately selected, wherein R^{4a} and R^{4b} are each separately selected from the group consisting of hydrogen, optionally substituted C₁₋₆alkyl, and aryl(CH₂)ₙ—;
each R^{5a} is separately selected from the group consisting of optionally substituted C₁₋₆alkyl and aryl(CH₂)ₙ—;
each R^{6a} is separately selected from the group consisting of optionally substituted C₁₋₆alkyl, and aryl(CH₂)ₙ—;
X¹ is (C(R²)₂)_{q}, 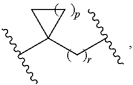
or X¹ is null;
Y¹ is selected from O (oxygen), S (sulfur), S(O), SO₂, NR², and C(R²)₂ with the proviso that when X¹ is null Y¹ is C(R²)₂;
X² is (C(R²)₂)_{q}, 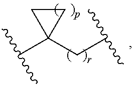
or X² is null;
Y² is selected from O (oxygen), S (sulfur), S(O), SO₂, NR², and C(R²)₂ with the proviso that when X² is null Y² is C(R²)₂;
each R² is separately selected, wherein R² is selected from the group consisting of hydrogen, C₁₋₆alkoxy, aryl, halo, hydroxy, R^{a}R^{b}N—, and C₁₋₆alkyl optionally substituted with up to 9 halo, or optionally two R² and the carbons to which they are attached are together a fused three- to eight-membered carbocyclic ring optionally containing one or two heteroatoms each independently selected from O (oxygen), N (nitrogen), and S (sulfur); wherein the three- to eight-membered carbocyclic ring is optionally substituted with one or more substituents selected from the group consisting of halo, hydroxyl, oxo, R^{x}R^{y}N, R^{x}R^{y}NC(═O), R^{x}R^{y}NC₁₋₆alkyl, heteroaryl, aryl, C₁₋₆alkyl optionally substituted with up to 9 halo, and C₁₋₆alkoxy optionally substituted with up to 9 halo;
each R^{a}R^{b}N is separately selected, wherein R^{a} and R^{b} are each separately selected from the group consisting of hydrogen, C₂₋₆alkenyl, and C₁₋₆alkyl;
L⁸ is selected from the group consisting of 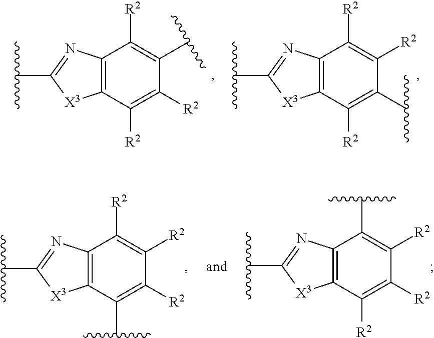
L⁹ is selected from the group consisting of 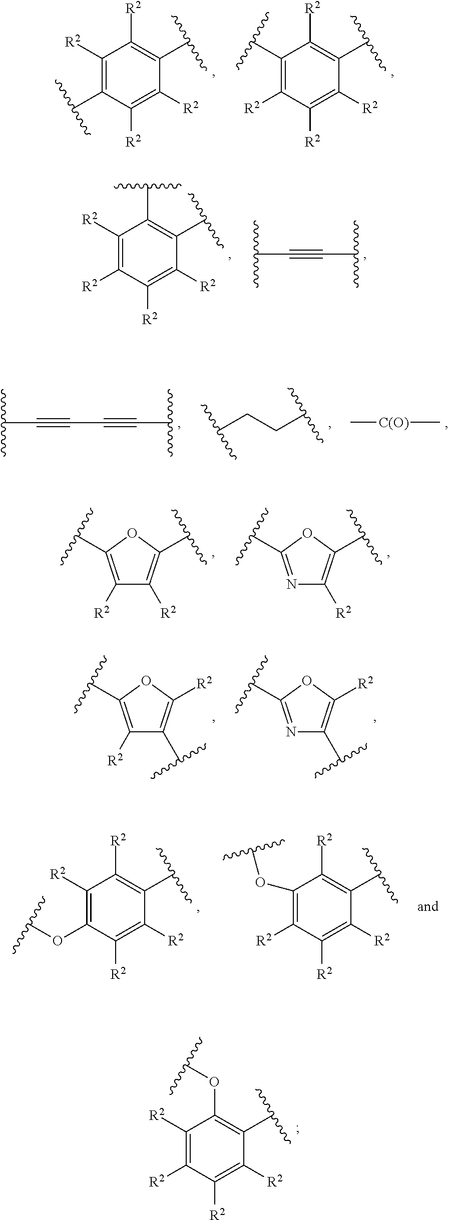
L¹⁰ is selected from the group consisting of 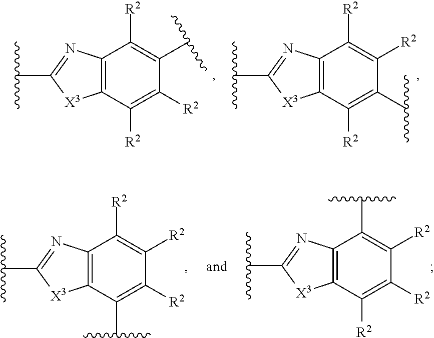
each X³ is separately selected from the group consisting of NH, NC₁₋₆alkyl, O (oxygen), and S (sulfur);
each m separately is 1 or 2;
each n separately is 0, 1 or 2;
each p separately is 1, 2, 3 or 4;
each q separately is 1, 2, 3, 4 or 5; and
each r separately is 0, 1, 2, 3, or 4,
wherein at least one of R^{2a}, R^{3a}, R^{4a}, R^{4b}, R^{5a}, and R^{6a} is a substituted C₁₋₆alkyl.

50. The compound of claim 49, wherein each R¹ is R^{1a}C(═O)—.

51. The compound of claim 49, wherein each R^{1a} is —CHR^{2a}NHR^{3b}.

52. The compound of claim 49, wherein each R^{2a} is optionally substituted C₁₋₆alkyl;
each R^{3b} is —C(═O)OR^{5a}; and
each R^{5a} is optionally substituted C₁₋₆alkyl.

53. The compound of claim 49, wherein at least one R^{1a} is —C(R^{2a})₂NR^{3a}R^{3b} and within said at least one R^{1a}, at least one R^{2a} is a substituted C₁₋₆alkyl.

54. The compound of claim 49, wherein at least one R^{1a} is —C(R^{2a})₂NR^{3a}R^{3b} wherein R^{3b} is —(CH₂)ₙC(═O)OR^{5a} and R^{5a} is a substituted C₁₋₆alkyl.

55. The compound of claim 49, wherein at least one of R^{2a}, R^{3a}, R^{4a}, R^{4b}, R^{5a}, and R^{6a} is C₁₋₆alkyl substituted with up to 9 halogen.

56. The compound of claim 49, wherein each R² is separately selected from the group consisting of hydrogen, C₁₋₆alkoxy, aryl, halo, hydroxy, R^{a}R^{b}N—, and C₁₋₆alkyl optionally substituted with up to 9 halo.

57. The compound of claim 49, wherein at least one of R^{2a}, R^{3a}, R^{4a}, R^{4b}, and R^{6a} is C₁₋₆alkyl substituted with up to 9 halogen.

58. The compound of claim 49, wherein L⁹ is selected from the group consisting of:

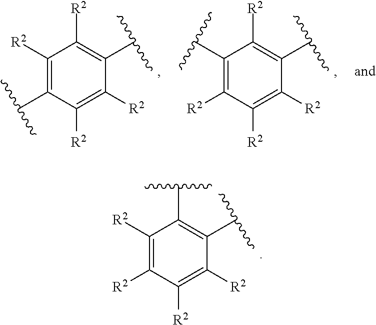

59. The compound of claim 49 having the structure of Formula Xa: 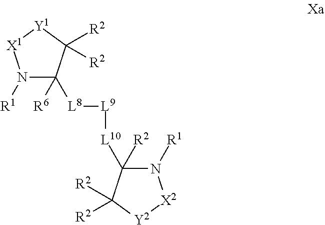
or a pharmaceutically acceptable salt thereof, wherein R⁶ is C₁₋₆alkyl optionally substituted with up to 9 halo.

60. The compound of claim 59, wherein R⁶ is methyl.

61. A compound having the structure of Formula XI: 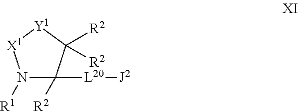
or a pharmaceutically acceptable salt thereof,
wherein:
R¹ is selected from the group consisting of hydrogen and R^{1a}C(═O)— and R^{1a}C(═S)—;
R^{1a} is selected from the group consisting of —C(R^{2a})₂NR^{3a}R^{3b}, alkoxyalkyl, C₁₋₆alkylOC(═O)—, C₁₋₆alkylOC(═O)C₁₋₆alkyl, C₁₋₆alkylC(═O)C₁₋₆alkyl, aryl, aryl(CH₂)ₙ—, aryl(CH₂)ₙO—, aryl(CH═CH)ₘ—, arylalkylO—, arylalkyl, arylOalkyl, cycloalkyl, (cycloalkyl)(CH═CH)ₘ—, (cycloalkyl)alkyl, cycloalkylOalkyl, heterocyclyl, heterocyclyl(CH═CH)ₘ—, heterocyclylalkoxy, heterocyclylalkyl, heterocyclylOalkyl, hydroxyalkyl, R^{c}R^{d}N—, R^{c}R^{d}N(CH₂)ₙ—, (R^{c}R^{d}N)(CH═CH)ₘ—, (R^{c}R^{d}N)alkyl, (R^{c}R^{d}N)C(═O)—, C₁₋₆alkoxy optionally substituted with up to 9 halo, and C₁₋₆alkyl optionally substituted with up to 9 halo, said aryl and heteroaryl each optionally substituted with cyano, halo, nitro, hydroxyl, C₁₋₆alkoxy optionally substituted with up to 9 halo, and C₁₋₆alkyl optionally substituted with up to 9 halo;
R^{c} and R^{d} are each separately selected from the group consisting of hydrogen, alkoxyC(═O)—, C₁₋₆alkyl, C₁₋₆alkylC(═O)—, C₁₋₆alkylsulfonyl, arylalkylOC(═O)—, arylalkyl, arylalkylC(═O)—, arylC(═O)—, arylsulfonyl, heterocyclylalkyl, heterocyclylalkylC(═O)—, heterocyclylC(═O)—, (R^{e}R^{f}N)alkyl, (R^{e}R^{f}N)alkylC(═O)—, and (R^{e}R^{f}N)C(═O)—, wherein the alkyl part of arylalkyl, arylalkylC(═O)—, heterocyclylalkyl, and heterocyclylalkylC(═O)— are each optionally substituted with one R^{e}R^{f}N— group; and wherein the aryl part of arylalkyl, arylalkylC(═O)—, arylC(═O)—, and arylsulfonyl, and the heterocyclyl part of heterocyclylalkyl, heterocyclylalkylC(═O)—, and heterocyclylC(═O)— are each optionally substituted with up to three substituents each independently selected from the group consisting of cyano, halo, nitro, C₁₋₆alkoxy optionally substituted with up to 9 halo, and C₁₋₆alkyl optionally substituted with up to 9 halo;
each R^{e}R^{f}N is separately selected, wherein R^{e} and R^{f} are each separately selected from the group consisting of hydrogen, C₁₋₆alkyl, aryl, arylalkyl, cycloalkyl, (cyclolalkyl)alkyl, heterocyclyl, heterocyclylalkyl, (R^{x}R^{y}N)alkyl, and (R^{x}R^{y}N)C(═O);
each R^{x}R^{y}N is separately selected, wherein R^{x} and R^{y} are each separately selected from hydrogen, C₁₋₆alkylOC(═O)—, C₁₋₆alkyl, C₁₋₆alkylC(═O)—, aryl, arylalkyl, cycloalkyl, and heterocyclyl;
R^{2a} is selected from the group consisting of hydrogen, optionally substituted C₁₋₆alkyl, aryl(CH₂)ₙ—, and heteroaryl(CH₂)ₙ—, said aryl and heteroaryl each optionally substituted with cyano, halo, nitro, hydroxyl, C₁₋₆alkoxy optionally substituted with up to 9 halo, and C₁₋₆alkyl optionally substituted with up to 9 halo, or C(R^{2a})₂ is 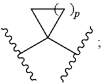
R^{3a} is selected from the group consisting of hydrogen, and optionally substituted C₁₋₆alkyl;
R^{3b} is selected from the group consisting of optionally substituted C₁₋₆alkyl, heteroaryl, —(CH₂)ₙC(═O)NR^{4a}R^{4b}, —(CH₂)ₙC(═O)OR^{5a}, and —(CH₂)ₙC(═O)R^{6a} said heteroaryl optionally substituted with one or more substituents selected from the group consisting of cyano, halo, nitro, hydroxyl, C₁₋₆alkoxy optionally substituted with up to 9 halo, and C₁₋₆alkyl optionally substituted with up to 9 halo;
R^{4a} and R^{4b} are each separately selected from the group consisting of hydrogen, optionally substituted C₁₋₆alkyl, and aryl(CH₂)ₙ—;
each R^{5a} is separately selected from the group consisting of optionally substituted C₁₋₆alkyl and aryl(CH₂)ₙ—;
each R^{6a} is separately selected from the group consisting of optionally substituted C₁₋₆alkyl, and aryl(CH₂)ₙ—; 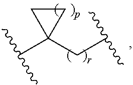
X¹ is (C(R²)₂)_{q}, or X¹ is null;
Y¹ is selected from O (oxygen), S (sulfur), S(O), SO₂, NR², and C(R²)₂ with the proviso that when X¹ is null Y¹ is C(R²)₂;
each R² is separately selected, wherein R² is selected from the group consisting of hydrogen, C₁₋₆alkoxy, aryl, halo, hydroxy, R^{a}R^{b}N—, and C₁₋₆alkyl optionally substituted with up to 9 halo, or optionally two R² and the carbons to which they are attached are together a fused three- to eight-membered carbocyclic ring optionally containing one or two heteroatoms each independently selected from O (oxygen), N (nitrogen), and S (sulfur); wherein the three- to eight-membered carbocyclic ring is optionally substituted with one or more substituents selected from the group consisting of halo, hydroxyl, oxo, R^{x}R^{y}N, R^{x}R^{y}NC(═O), R^{x}R^{y}NC₁₋₆alkyl, heteroaryl, aryl, C₁₋₆alkyl optionally substituted with up to 9 halo, and C₁₋₆alkoxy optionally substituted with up to 9 halo, or optionally two geminal R² and the carbon to which they are attached are together carbonyl, or optionally two geminal R² and the carbon to which they are attached are together a three- to six-membered carbocyclic ring optionally substituted with up to two C₁₋₆alkyl groups;
each R^{a}R^{b}N is separately selected, wherein R^{a} and R^{b} are each separately selected from the group consisting of hydrogen, C₂₋₆alkenyl, and C₁₋₆alkyl;
L²⁰ is selected from the group consisting of Q¹-Q², Q³-Q⁴, 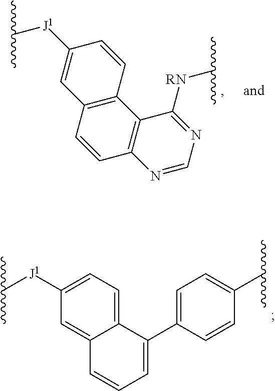
Q¹ is selected from the group consisting of J⁵, J⁴-J⁵, J¹-J⁵-J¹⁰, J¹-J⁵-J³, 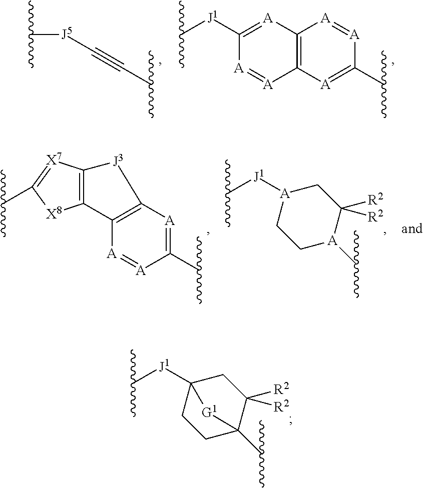
Q² is 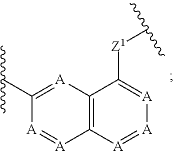
Q³ is selected from the group consisting of, J⁵, J¹-J⁵, J¹-J⁵-J¹⁰, J¹-J⁵-J³, 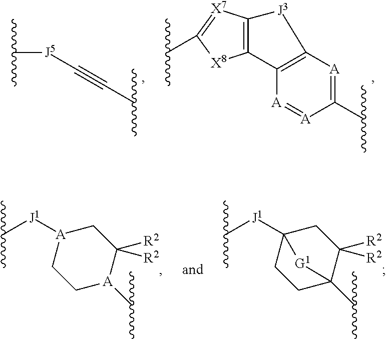
Q⁴ is selected from the group consisting of 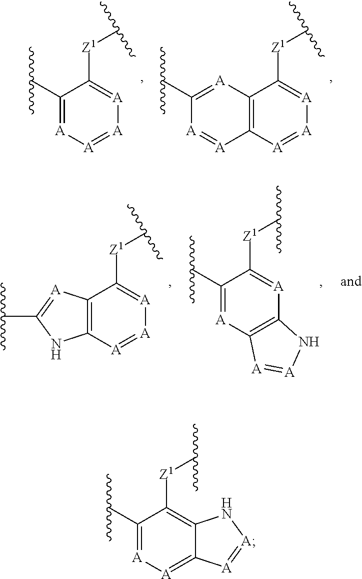
Z¹ is selected from the group consisting of O (oxygen), S (sulfur), NR, and C(R²)₂;
each A is separately selected from the group consisting of CR³ and N (nitrogen);
each R³ is separately selected from the group consisting of hydrogen, C₁₋₆alkoxy, C₁₋₆alkylOC₁₋₆alkyl, C₁₋₆alkylOC(═O)—, arylalkylOC(═O)—, —COOH, halo, hydroxy, R^{a}R^{b}N—, (R^{a}R^{b}N)alkyl, (R^{a}R^{b}N)C(═O)—, and C₁₋₆alkyl optionally substituted with up to 9 halo and up to 5 hydroxy;
G¹ is —CH₂— or —CH₂CH₂—;
J¹ is 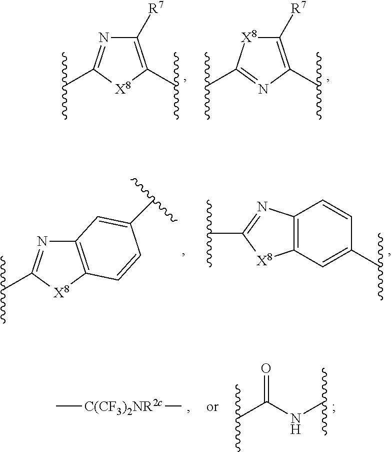
R^{2c} is selected from the group consisting of hydrogen, C₁₋₆alkyl, C₂₋₆alkenyl, C₂₋₆alkynyl, C₃₋₇cycloalkyl, aryl, arylalkyl, heteroaryl, heteroarylalkyl, heterocyclyl, and heterocyclylalkyl, said alkyl optionally substituted with R^{e}R^{f}N—, alkoxy, or C₁₋₆alkylS—;
J² is aryl, heteroaryl, heterocyclyl, or polycyclic hydrocarbon, each optionally substituted one or more substituents independently selected from the group consisting of halo, hydroxyl, R^{x}R^{y}N, R^{x}R^{y}NC(═O), R^{x}R^{y}NC₁₋₆alkyl, heteroaryl, aryl, C₁₋₆alkyl optionally substituted with up to 9 halo, and C₁₋₆alkoxy optionally substituted with up to 9 halo, said substituent aryl and heteroaryl are each optionally substituted with one or more R¹⁴;
each R¹⁴ is separately selected from the group consisting of hydroxy, C₁₋₆alkoxy optionally substituted with up to 9 fluoro, C₁₋₆alkylOC₁₋₆alkyl, C₁₋₆alkylOC(═O)—, arylalkylOC(═O)—, —COOH, halo, and C₁₋₆alkyl optionally substituted with up to 9 halo;
J³ is C₂₋₄ alkyl, NH, O (oxygen), —NHC(O)—, S (sulfur), —(CH₂)ₙX⁸(CH₂)ₘ—, or —X⁷═X⁷;
J⁴ is 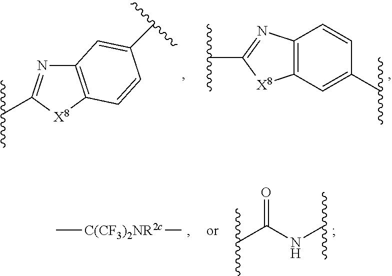
J⁵ is aryl, heteroaryl, heterocyclyl, or polycyclic hydrocarbon, each optionally substituted one or more R³;
each X⁷ is separately selected from the group consisting of N (nitrogen), and CR²;
each X⁸ is separately selected from the group consisting of NH, NC₁₋₆alkyl, O (oxygen), and S (sulfur);
J¹⁰ is —C(R²)₂—, —NR—, oxygen (O), or sulfur (S);
each m separately is 1 or 2;
each n separately is 0, 1 or 2;
each p separately is 1, 2, 3 or 4;
each q separately is 1, 2, 3, 4 or 5;
each r separately is 0, 1, 2, 3, or 4;
each R is separately selected from the group consisting of hydrogen, C₁₋₆alkylOC(═O)—, arylalkylOC(═O)—, (R^{a}R^{b}N)C(═O)—, and C₁₋₆alkyl optionally substituted with up to 9 halo; and
R⁷ is selected from the group consisting of hydrogen, halo, C₁₋₆alkylOC(═O)—, arylalkylOC(═O)—, —COOH, (R^{a}R^{b}N)C(═O)—, trialkylsilylalkylOalkyl, and C₁₋₆alkyl optionally substituted with up to 9 halo.

62. The compound of claim 61, wherein L²⁰ is selected from the group consisting of 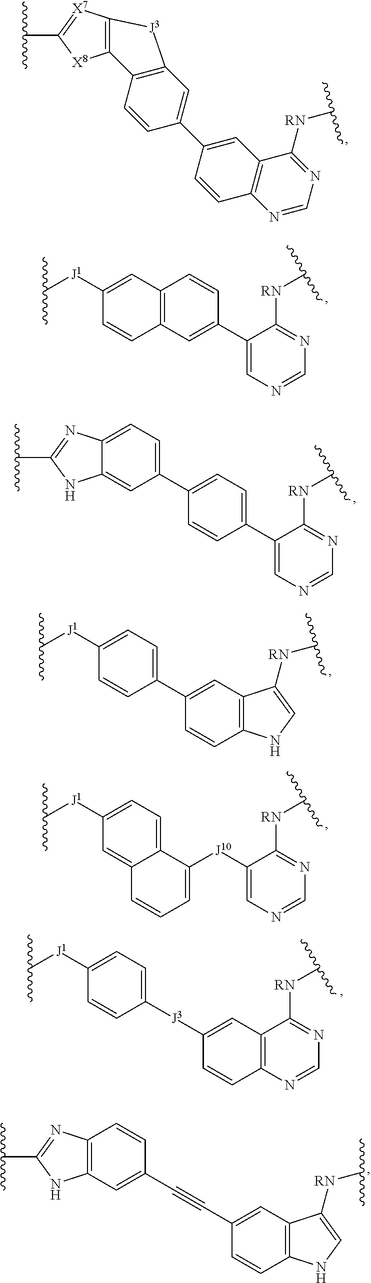 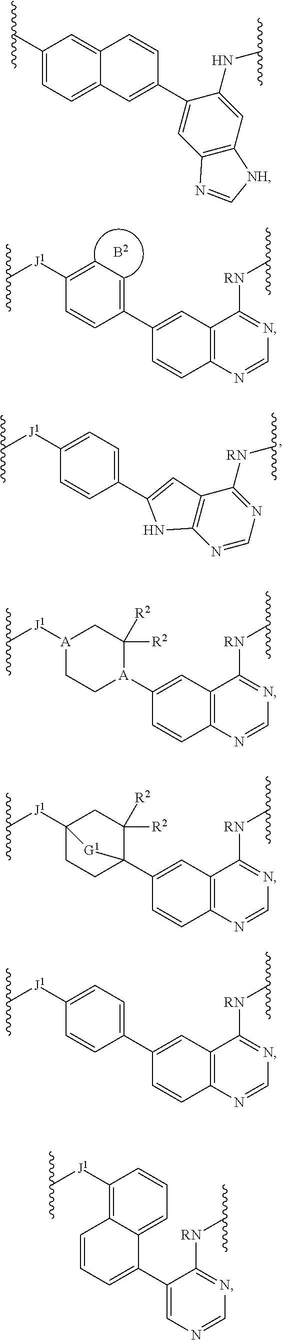 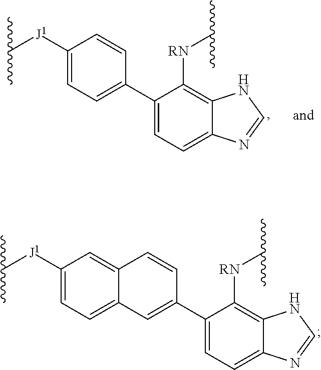
B² is an aliphatic ring optionally including O (oxygen), S (sulfur), or NH, or an aromatic ring optionally including N (nitrogen), said aliphatic or aromatic ring in the definition of B₂ is optionally substituted with one or more R^{g}; and
each R^{g} is separately selected from the group consisting of halo, hydroxy, C₁₋₆alkoxy optionally substituted with up to 9 fluoro, and C₁₋₆alkyl optionally substituted with up to 9 halo.

63. The compound of claim 61, wherein Q¹ is selected from the group consisting of 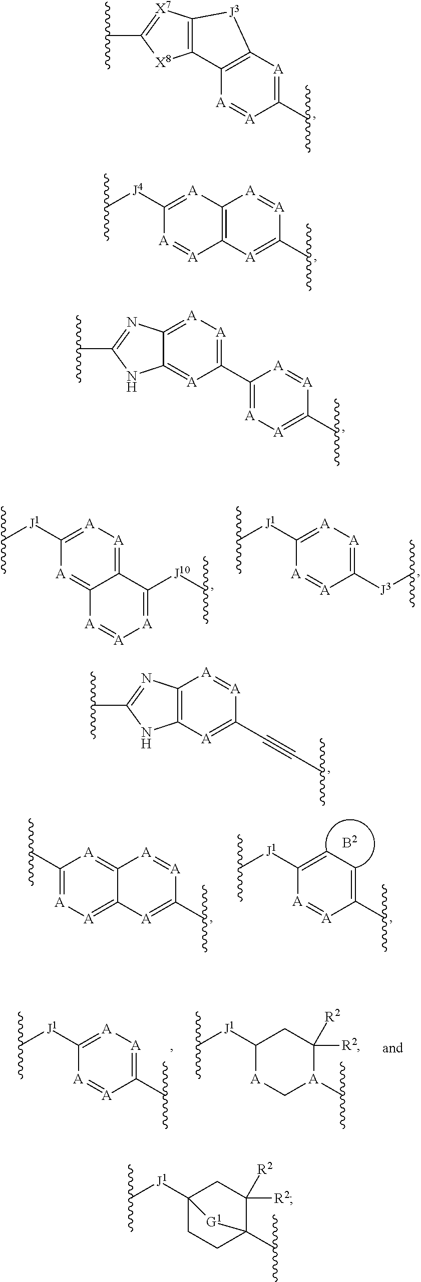
B² is an aliphatic ring optionally including O (oxygen), S (sulfur), or NH, or an aromatic ring optionally including N (nitrogen), said aliphatic or aromatic ring in the definition of B² is optionally substituted with one or more R^{g}; and
each R^{g} is separately selected from the group consisting of halo, hydroxy, C₁₋₆alkoxy optionally substituted with up to 9 fluoro, and C₁₋₆alkyl optionally substituted with up to 9 halo.

64. The compound of claim 61, wherein Q³ is selected from the group consisting of

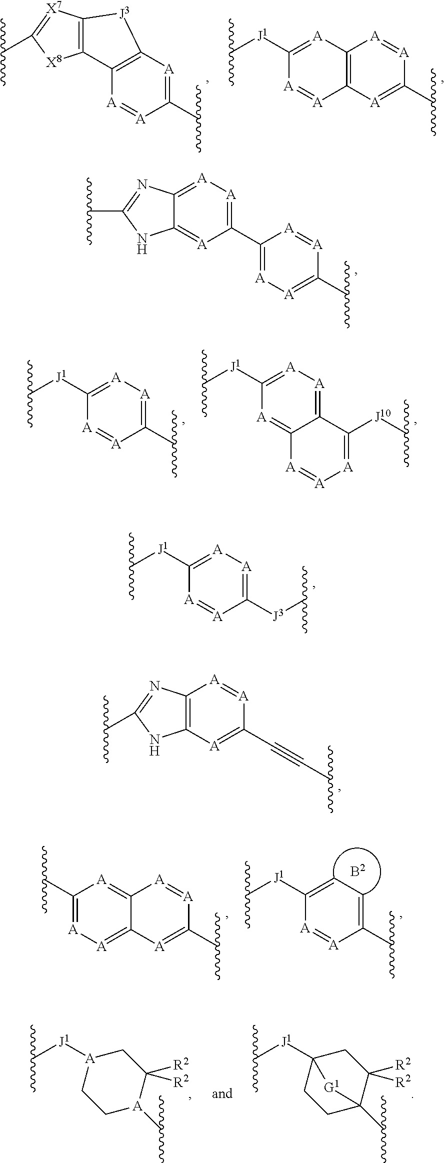

65. The compound of claim 61, having the structure: 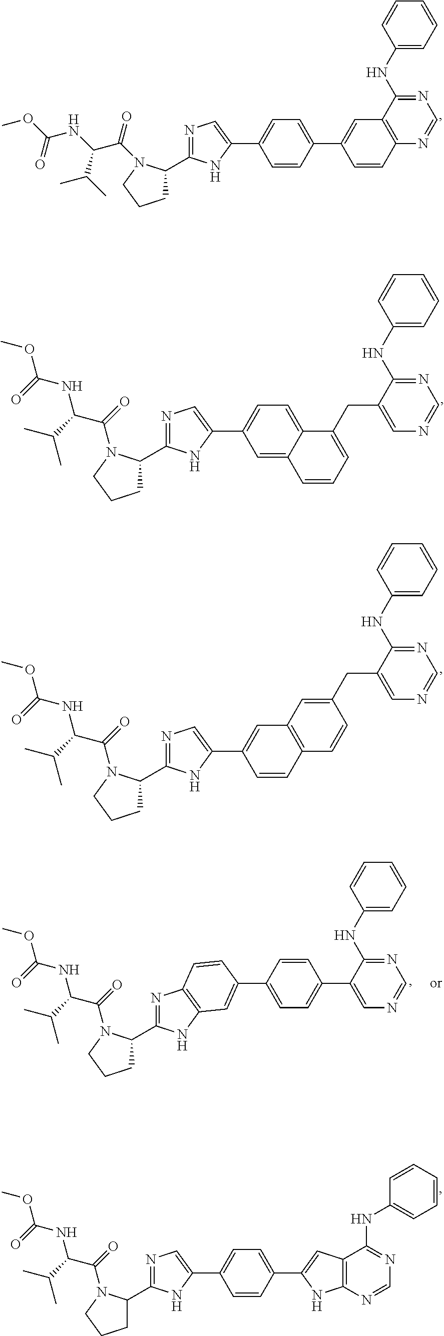 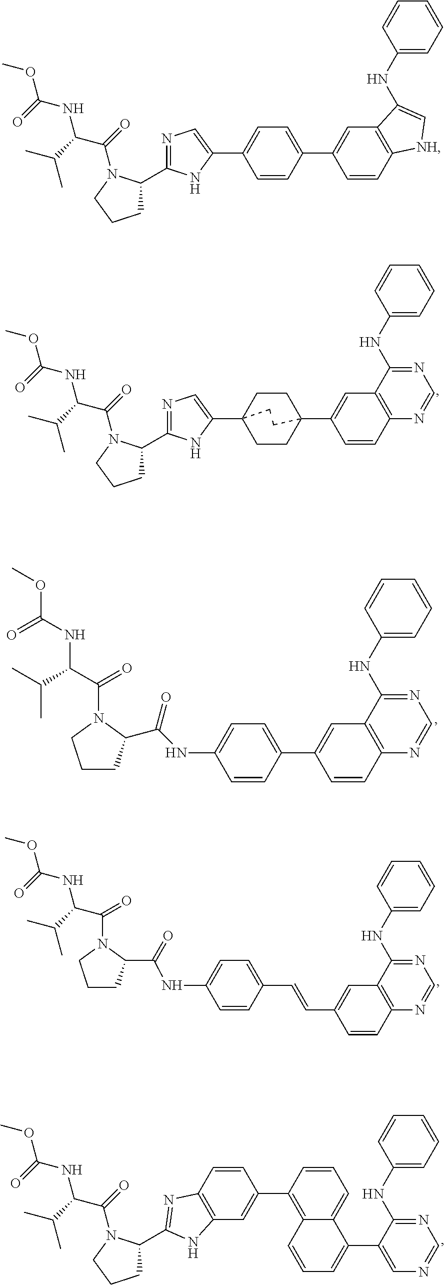 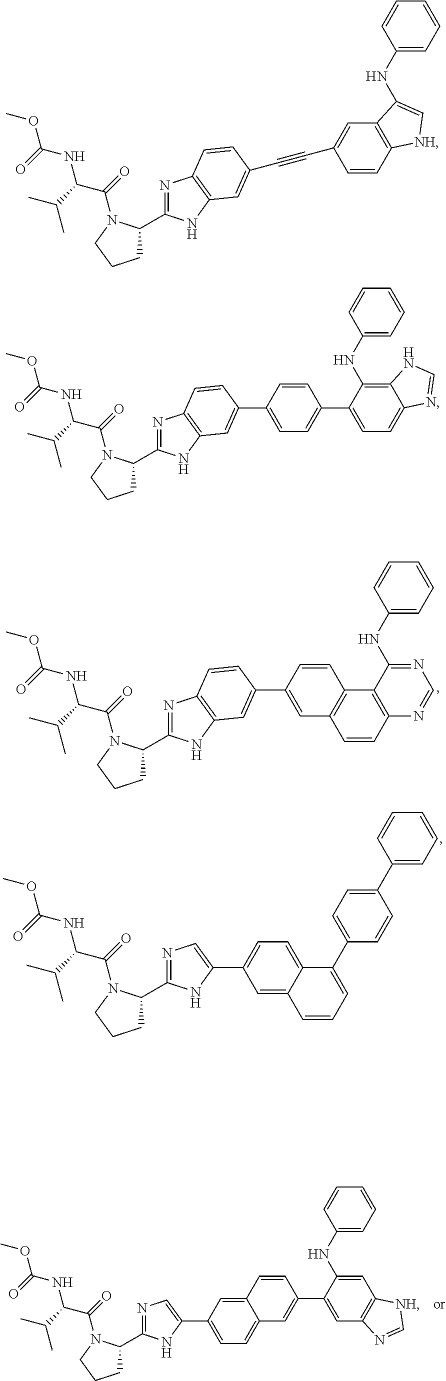 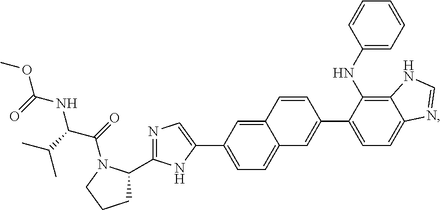
or a pharmaceutically acceptable salt thereof.

66. A compound having the structure of Formula XII: 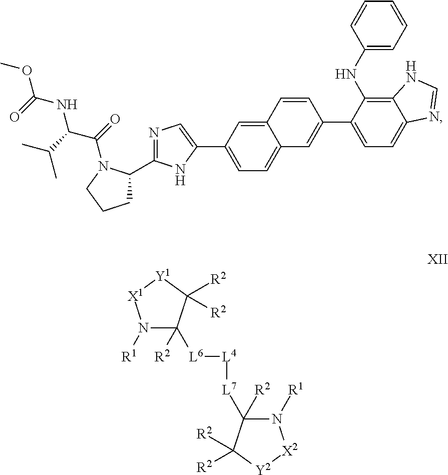
or a pharmaceutically acceptable salt thereof,
wherein:
each R¹ is separately selected from the group consisting of hydrogen, R^{1a}C(═O)— and R^{1a}C(═S)—;
each R^{1a} is separately selected from the group consisting of —C(R^{2a})₂NR^{3a}R^{3b}, alkoxyalkyl, C₁₋₆alkylOC(═O)—, C₁₋₆alkylOC(═O)C₁₋₆alkyl, C₁₋₆alkylC(═O)C₁₋₆alkyl, aryl, aryl(CH₂)ₙ—, aryl(CH₂)ₙO—, aryl(CH═CH)ₘ—, arylalkylO—, arylalkyl, arylOalkyl, cycloalkyl, (cycloalkyl)(CH═CH)ₘ—, (cycloalkyl)alkyl, cycloalkylOalkyl, heterocyclyl, heterocyclyl(CH═CH)ₘ—, heterocyclylalkoxy, heterocyclylalkyl, heterocyclylOalkyl, hydroxyalkyl, R^{c}R^{d}N—, R^{c}R^{d}N(CH₂)ₙ—, (R^{c}R^{d}N)(CH═CH)ₘ—, (R^{c}R^{d}N)alkyl, (R^{c}R^{d}N)C(═O)—, C₁₋₆alkoxy optionally substituted with up to 9 halo, and C₁₋₆alkyl optionally substituted with up to 9 halo, said aryl and heteroaryl each optionally substituted with cyano, halo, nitro, hydroxyl, C₁₋₆alkoxy optionally substituted with up to 9 halo, and C₁₋₆alkyl optionally substituted with up to 9 halo;
each R^{c}R^{d}N is separately selected, wherein R^{c} and R^{d} are each separately selected from hydrogen, alkoxyC(═O)—, C₁₋₆alkyl, C₁₋₆alkylC(═O)—, C₁₋₆alkylsulfonyl, arylalkylOC(═O)—, arylalkyl, arylalkylC(═O)—, arylC(═O)—, arylsulfonyl, heterocyclylalkyl, heterocyclylalkylC(═O)—, heterocyclylC(═O)—, (R^{e}R^{f}N)alkyl, (R^{e}R^{f}N)alkylC(═O)—, and (R^{e}R^{f}N)C(═O)—, wherein the alkyl part of arylalkyl, arylalkylC(═O)—, heterocyclylalkyl, and heterocyclylalkylC(═O)— are each optionally substituted with one R^{e}R^{f}N— group; and wherein the aryl part of arylalkyl, arylalkylC(═O)—, arylC(═O)—, and arylsulfonyl, and the heterocyclyl part of heterocyclylalkyl, heterocyclylalkylC(═O)—, and heterocyclylC(═O)— are each optionally substituted with up to three substituents each independently selected from the group consisting of cyano, halo, nitro, C₁₋₆alkoxy optionally substituted with up to 9 halo, and C₁₋₆alkyl optionally substituted with up to 9 halo;
each R^{e}R^{f}N is separately selected, wherein R^{e} and R^{f} are each separately selected from hydrogen, C₁₋₆alkyl, aryl, arylalkyl, cycloalkyl, (cyclolalkyl)alkyl, heterocyclyl, heterocyclylalkyl, (R^{x}R^{y}N)alkyl, and (R^{x}R^{y}N)C(═O)—;
each R^{x}R^{y}N is separately selected, wherein R^{x} and R^{y} are each separately selected from hydrogen, C₁₋₆alkylOC(═O)—, C₁₋₆alkyl, C₁₋₆alkylC(═O)—, aryl, arylalkyl, cycloalkyl, and heterocyclyl;
each C(R^{2a})₂ is separately selected, wherein each R^{2a} is separately selected from the group consisting of hydrogen, C₁₋₆alkyl optionally substituted with up to 9 halo, aryl(CH₂)ₙ—, and heteroaryl(CH₂)ₙ—, said aryl and heteroaryl each optionally substituted with cyano, halo, nitro, hydroxyl, C₁₋₆alkoxy optionally substituted with up to 9 halo, and C₁₋₆alkyl optionally substituted with up to 9 halo, or C(R^{2a})₂ is 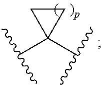
each R^{3a} is separately selected from the group consisting of hydrogen, and optionally substituted C₁₋₆alkyl;
each R^{3b} is separately selected from the group consisting of optionally substituted C₁₋₆alkyl, heteroaryl, —(CH₂)ₙC(═O)NR^{4a}R^{4b}, —(CH₂)ₙC(═O)OR^{5a}, and —(CH₂)ₙC(═O)R^{6a} said heteroaryl optionally substituted with cyano, halo, nitro, hydroxyl, C₁₋₆alkoxy optionally substituted with up to 9 halo, and C₁₋₆alkyl optionally substituted with up to 9 halo;
each R^{4a}R^{4b}N is separately selected, wherein R^{4a} and R^{4b} are each separately selected from the group consisting of hydrogen, optionally substituted C₁₋₆alkyl, and aryl(CH₂)ₙ—;
each R^{5a} is separately selected from the group consisting of optionally substituted C₁₋₆alkyl, and aryl(CH₂)ₙ—;
each R^{6a} is separately selected from the group consisting of optionally substituted C₁₋₆alkyl, and aryl(CH₂)ₙ—;
X¹ is (C(R²)₂)_{q}, 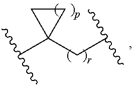
or X¹ is null;
Y¹ is selected from O (oxygen), S (sulfur), S(O), SO₂, NR², and C(R²)₂ with the proviso that when X¹ is null Y¹ is C(R²)₂;
X² is (C(R²)₂)_{q}, 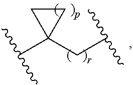
or X² is null;
Y² is selected from O (oxygen), S (sulfur), S(O), SO₂, NR², and C(R²)₂ with the proviso that when X² is null Y² is C(R²)₂;
each R² is separately selected, wherein R² is selected from the group consisting of hydrogen, C₁₋₆alkoxy, C₁₋₆alkyl, aryl, halo, hydroxy, R^{a}R^{b}N—, and C₁₋₆alkyl optionally substituted with up to 9 halo, or optionally two vicinal R² and the carbons to which they are attached are together a fused three- to six-membered carbocyclic ring optionally substituted with up to two C₁₋₆alkyl groups;
each R^{a}R^{b}N is separately selected, wherein R^{a} and R^{b} are each separately selected from the group consisting of hydrogen, C₂₋₆alkenyl, and C₁₋₆alkyl;
each A is separately selected from the group consisting of CR³ and N (nitrogen);
L⁴ is selected from the group consisting of -(J²)ₛ-(L⁵)ₛ-(J²)ₛ-(L⁵)ₛ-J²-, 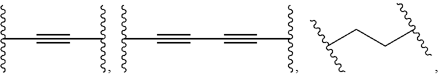
—C(═O)—, O (oxygen), —OC(R²)₂—, 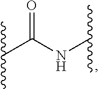
—C(CF₃)₂NR^{2c}—, NH, and —(CH═CH)—;
J² is aryl, heteroaryl, heterocyclyl, cycloalkyl, cycloalkenyl, or polycyclic hydrocarbon, each optionally substituted with one or more R¹⁵;
each R¹⁴ is separately selected from the group consisting of hydroxy, C₁₋₆alkoxy optionally substituted with up to 9 fluoro, C₁₋₆alkylOC₁₋₆alkyl, C₁₋₆alkylOC(═O)—, arylalkylOC(═O)—, —COOH, halo, and C₁₋₆alkyl optionally substituted with up to 9 halo;
each R¹⁵ is separately selected from the group consisting of halo, hydroxy, C₁₋₆alkylOC₁₋₆alkyl, C₁₋₆alkylOC(═O)—, arylalkylOC(═O)—, —COOH, R⁹, R^{x}R^{y}N, R^{x}R^{y}NC(═O), R^{x}R^{y}NC₁₋₆alkyl, heteroaryl, aryl, C₁₋₆alkyl optionally substituted with up to 9 halo, C₁₋₆alkyl substituted with up to 5 hydroxy, C₁₋₆alkoxy optionally substituted with up to 9 halo, C₁₋₆haloalkyl, R^{a}R^{b}N—, (R^{a}R^{b}N)alkyl, (R^{a}R^{b}N)C(═O)—, C₁₋₆alkyl substituted with up to 5 hydroxy, said substituent aryl and heteroaryl are each optionally substituted with one or more R¹⁴, or optionally two vicinal R¹⁵ and the carbons to which they are attached are together a fused three- to six-membered carbocyclic ring optionally substituted with up to two C₁₋₆alkyl groups, or optionally two geminal R¹⁵ and the carbon to which they are attached are together a three- to six-membered carbocyclic ring optionally substituted with up to two C₁₋₆alkyl groups, or optionally two geminal R¹⁵ are together oxo;
each L⁵ is separately selected from the group consisting of 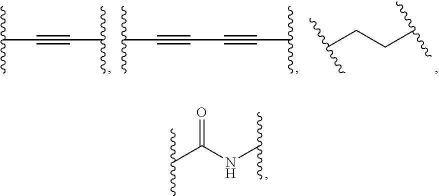
—C(CF₃)₂NR^{2c}—, 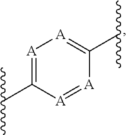
C(R²)₂, —C(R²)₂O—, —C(═O)—, O (oxygen), NH, and —(CH═CH)—;
R^{2c} is selected from the group consisting of hydrogen, C₁₋₆alkyl, C₂₋₆alkenyl, C₂₋₆alkynyl, C₃₋₇cycloalkyl, aryl, arylalkyl, heteroaryl, heteroarylalkyl, heterocyclyl, and heterocyclylalkyl, said alkyl optionally substituted with R^{e}R^{f}N—, alkoxy, or C₁₋₆alkylS—;
L⁶ is selected from the group consisting of 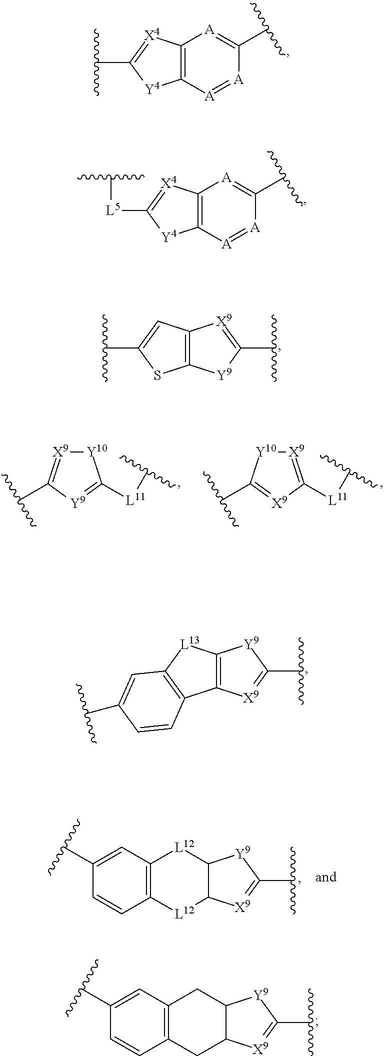
L⁷ is selected from the group consisting of 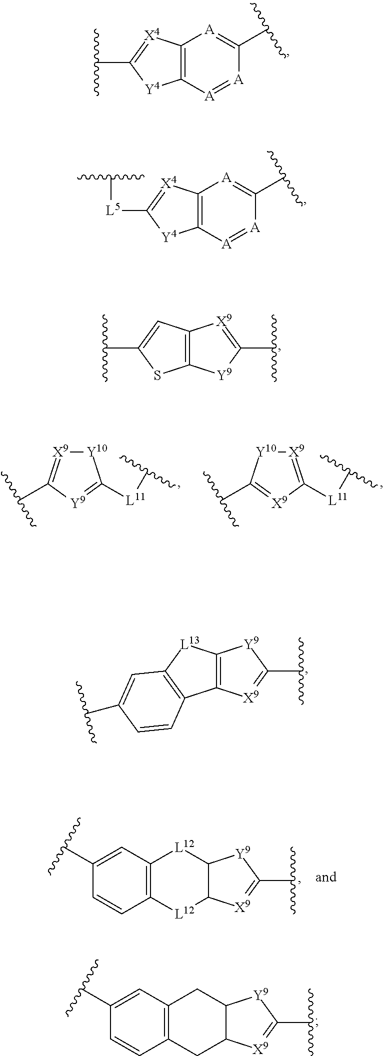
each X⁴ is separately selected from the group consisting of CR⁴ and N (nitrogen), wherein if X₄ is N (nitrogen) then Y₄ is not NH;
each X⁶ is separately selected from the group consisting of N (nitrogen), and CR⁸;
each Y⁴ is separately selected from the group consisting of C(R⁴)₂, NR⁴, O (oxygen), and S (sulfur);
each Y⁹ is separately selected from the group consisting of —NH—, O (oxygen), and S (sulfur);
each X⁹ is separately selected from the group consisting of CH and N (nitrogen), wherein if X⁹ is N (nitrogen) then Y⁹ is not NH;
each Y¹⁰ is separately selected from the group consisting of —CH₂— and —NH—;
each L¹¹ is separately selected from the group consisting of 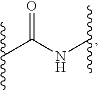
—C(CF₃)₂NR^{2c}—, and NH;
each L¹² is separately selected from the group consisting of —CH₂— and —CH₂CH₂—;
each L¹³ is separately selected from the group consisting of —CH₂—, —N═CH—, —CH═CH—, —CH₂CH₂—, —(CH₂)ₘNR⁴(CH₂)ₙ— and —(CH₂)ₘO(CH₂)ₙ—;
each m separately is 1 or 2;
each n separately is 0, 1 or 2;
each p separately is 1, 2, 3 or 4;
each q separately is 1, 2, 3, 4 or 5;
each r separately is 0, 1, 2, 3, or 4;
each s separately is 0 or 1;
each R³ is separately selected from the group consisting of hydrogen, C₁₋₆alkoxy, C₁₋₆alkylOC₁₋₆alkyl, C₁₋₆alkylOC(═O)—, arylalkylOC(═O)—, —COOH, halo, hydroxy, R^{a}R^{b}N—, (R^{a}R^{b}N)alkyl, (R^{a}R^{b}N)C(═O)—, and C₁₋₆alkyl optionally substituted with up to 9 halo and up to 5 hydroxy;
each R⁴ is separately selected from the group consisting of H (hydrogen), C₁₋₆alkoxy, C₁₋₆alkylOC₁₋₆alkyl, C₁₋₆alkylOC(═O)—, arylalkylOC(═O)—, —COOH, halo, C₁₋₆haloalkyl, hydroxy, R^{a}R^{b}N—, (R^{a}R^{b}N)alkyl, (R^{a}R^{b}N)C(═O)—, and C₁₋₆alkyl optionally substituted with up to 9 halo and up to 5 hydroxy, or optionally two geminal R⁴ are together oxo;
R⁹ is selected from the group consisting of hydrogen and —C(═O)R^{9a};
R^{9a} is selected from the group consisting of —NR^{9b}R^{9c}, —OR^{9d}, C₁₋₆alkyl optionally substituted with up to 9 halo, and optionally substituted aryl;
R^{9b} is selected from the group consisting of hydrogen, C₁₋₆alkyl optionally substituted with up to 9 halo, and optionally substituted aryl;
R^{9c} is selected from the group consisting of C₁₋₆alkyl optionally substituted with up to 9 halo, and optionally substituted aryl; and
R^{9d} is selected from the group consisting of C₁₋₆alkyl optionally substituted with up to 9 halo, and optionally substituted aryl,
with the proviso that L⁶-L⁴-L⁷ is not

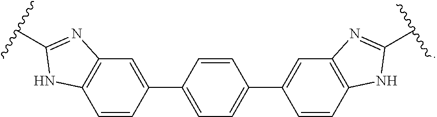

67. The compound of claim 66, wherein
L⁴ is selected from the group consisting of 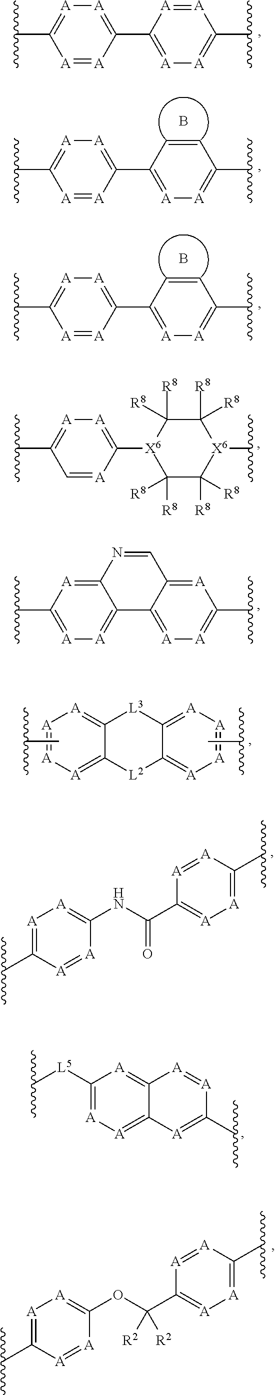 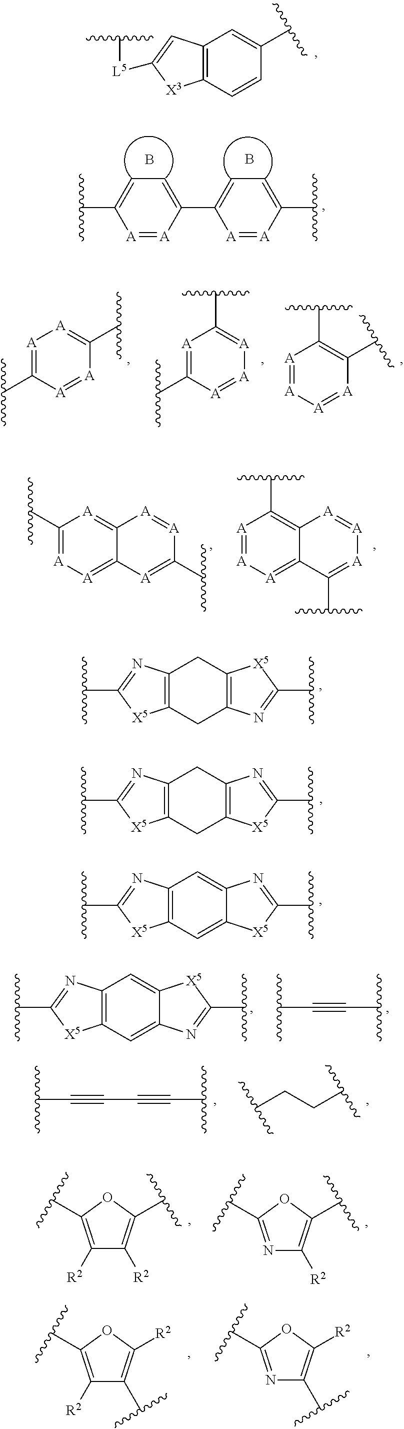 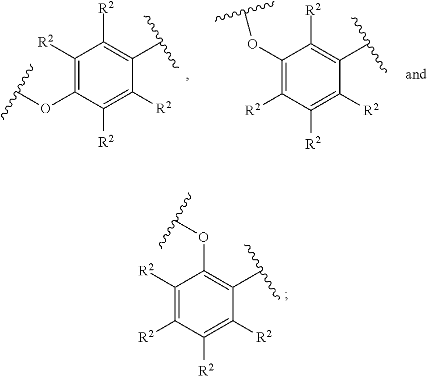
L² is selected from the group consisting of —C(═O)—, —(CH₂CH₂)—, —(CH₂O)—, —(CH₂S)—, —(CH═CH)—, —(CH═N)—, —NH—, O (oxygen), S (sulfur), and —CH₂—;
L³ is selected from the group consisting of 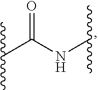
—(NR⁹)—, O (oxygen), S (sulfur), and —CH₂—;
L⁵ is selected from the group consisting of 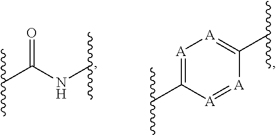
O (oxygen), NH, and —(CH═CH)—;
each X³ is separately selected from the group consisting of NH, O (oxygen), and S (sulfur);
each X⁵ is separately selected from the group consisting of —NH—, O (oxygen), S (sulfur), and —CH₂—;
each X⁶ is separately selected from the group consisting of N (nitrogen), and CR⁸;
each R⁸ is separately selected from the group consisting of hydrogen, C₁₋₆alkylOC₁₋₆alkyl, C₁₋₆alkylOC(═O)—, arylalkylOC(═O)—, halo, (R^{a}R^{b}N)alkyl, (R^{a}R^{b}N)C(═O)—, and C₁₋₆alkyl optionally substituted with up to 9 halo and up to 5 hydroxy;
each B is separately selected, wherein B is a fused optionally substituted saturated or unsaturated three- to seven-membered carbocyclic ring or a fused optionally substituted saturated or unsaturated three- to seven-membered heterocyclic ring, each optionally substituted with one or more R⁴ and;
each R⁴ is separately selected from the group consisting of C₁₋₆alkoxy, C₁₋₆alkylOC₁₋₆alkyl, C₁₋₆alkylOC(═O)—, arylalkylOC(═O)—, —COOH, halo, C₁₋₆haloalkyl, hydroxy, R^{a}R^{b}N—, (R^{a}R^{b}N)alkyl, (R^{a}R^{b}N)C(═O)—, and C₁₋₆alkyl optionally substituted with up to 9 halo and up to 5 hydroxy, or optionally two geminal R⁴ are together oxo.

68. The compound of claim 66 having the structure of Formula XIIa: 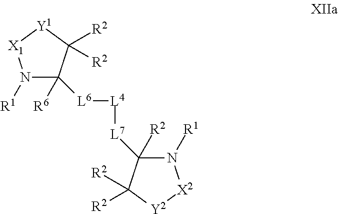
or a pharmaceutically acceptable salt thereof, wherein R⁶ is C₁₋₆alkyl optionally substituted with up to 9 halo.

69. The compound of claim 68, wherein R⁶ is methyl.

70. A compound having the structure of Formula XIII: 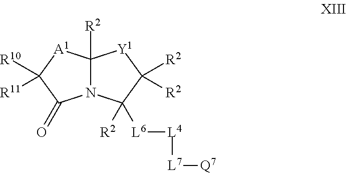
or a pharmaceutically acceptable salt thereof,
wherein:
each R¹ is separately selected from the group consisting of hydrogen, R^{1a}C(═O)— and R^{1a}C(═S)—;
each R^{1a} is separately selected from the group consisting of —C(R^{2a})₂NR^{3a}R^{3b}, alkoxyalkyl, C₁₋₆alkylOC(═O)—, C₁₋₆alkylOC(═O)C₁₋₆alkyl, C₁₋₆alkylC(═O)C₁₋₆alkyl, aryl, aryl(CH₂)ₙ—, aryl(CH₂)ₙO, aryl(CH═CH)ₘ—, arylalkylO—, arylalkyl, arylOalkyl, cycloalkyl, (cycloalkyl)(CH═CH)ₘ—, (cycloalkyl)alkyl, cycloalkylOalkyl, heterocyclyl, heterocyclyl(CH═CH)ₘ—, heterocyclylalkoxy, heterocyclylalkyl, heterocyclylOalkyl, hydroxyalkyl, R^{c}R^{d}N—, R^{c}R^{d}N(CH₂)ₙ—, (R^{c}R^{d}N)(CH═CH)ₘ—, (R^{c}R^{d}N)alkyl, (R^{c}R^{d}N)C(═O)—, C₁₋₆alkoxy optionally substituted with up to 9 halo, and C₁₋₆alkyl optionally substituted with up to 9 halo, said aryl and heteroaryl each optionally substituted with cyano, halo, nitro, hydroxyl, C₁₋₆alkoxy optionally substituted with up to 9 halo, and C₁₋₆alkyl optionally substituted with up to 9 halo;
each C(R^{2a})₂ is separately selected, wherein each R^{2a} is separately selected from the group consisting of hydrogen, C₁₋₆alkyl optionally substituted with up to 9 halo, aryl(CH₂)ₙ—, and heteroaryl(CH₂)ₙ—, said aryl and heteroaryl each optionally substituted with cyano, halo, nitro, hydroxyl, C₁₋₆alkoxy optionally substituted with up to 9 halo, and C₁₋₆alkyl optionally substituted with up to 9 halo, or C(R^{2a})₂ is 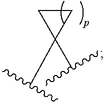
each R^{3a} is separately selected from the group consisting of hydrogen, and optionally substituted C₁₋₆alkyl;
each R^{3b} is separately selected from the group consisting of optionally substituted C₁₋₆alkyl, heteroaryl, —(CH₂)ₙC(═O)NR^{4a}R^{4b}, —(CH₂)ₙC(═O)OR^{5a}, and —(CH₂)ₙC(═O)R^{6a} said heteroaryl optionally substituted with cyano, halo, nitro, hydroxyl, C₁₋₆alkoxy optionally substituted with up to 9 halo, and C₁₋₆alkyl optionally substituted with up to 9 halo;
each R^{4a}R^{4b}N is separately selected, wherein R^{4a} and R^{4b} are each separately selected from the group consisting of hydrogen, optionally substituted C₁₋₆alkyl, and aryl(CH₂)ₙ—;
each R^{5a} is separately selected from the group consisting of optionally substituted C₁₋₆alkyl, and aryl(CH₂)ₙ—;
each R^{6a} is separately selected from the group consisting of optionally substituted C₁₋₆alkyl, and aryl(CH₂)ₙ—;
each R¹⁰ is R^{c}R^{d}N—;
each R¹¹ is separately selected from the group consisting of H (hydrogen), alkoxyalkyl, C₁₋₆alkylOC(═O)C₁₋₆alkyl, C₁₋₆alkylC(═O)C₁₋₆alkyl, aryl(CH₂)ₙ—, arylalkyl, arylOalkyl, cycloalkyl, (cycloalkyl)alkyl, cycloalkylOalkyl, heterocyclylalkyl, heterocyclylOalkyl, hydroxyalkyl, R^{c}R^{d}N(CH₂)ₙ—, (R^{c}R^{d}N)alkyl, and C₁₋₆alkyl optionally substituted with up to 9 halo;
each R^{e}R^{f}N is separately selected, wherein R^{c} and R^{d} are each separately selected from hydrogen, alkoxyC(═O)—, C₁₋₆alkyl, C₁₋₆alkylC(═O)—, C₁₋₆alkylsulfonyl, arylalkylOC(═O)—, arylalkyl, arylalkylC(═O)—, arylC(═O)—, arylsulfonyl, heterocyclylalkyl, heterocyclylalkylC(═O)—, heterocyclylC(═O)—, (R^{e}R^{f}N)alkyl, (R^{e}R^{f}N)alkylC(═O)—, and (R^{e}R^{f}N)C(═O)—, wherein the alkyl part of arylalkyl, arylalkylC(═O)—, heterocyclylalkyl, and heterocyclylalkylC(═O)— are each optionally substituted with one R^{e}R^{f}N— group; and wherein the aryl part of arylalkyl, arylalkylC(═O)—, arylC(═O)—, and arylsulfonyl, and the heterocyclyl part of heterocyclylalkyl, heterocyclylalkylC(═O)—, and heterocyclylC(═O)— are each optionally substituted with up to three substituents each independently selected from the group consisting of cyano, halo, nitro, C₁₋₆alkoxy optionally substituted with up to 9 halo, and C₁₋₆alkyl optionally substituted with up to 9 halo;
each R^{e}R^{f}N is separately selected, wherein R^{e} and R^{f} are each separately selected from hydrogen, C₁₋₆alkyl, aryl, arylalkyl, cycloalkyl, (cyclolalkyl)alkyl, heterocyclyl, heterocyclylalkyl, (R^{x}R^{y}N)alkyl, and (R^{x}R^{y}N)C(═O)—;
each R^{x}R^{y}N is separately selected, wherein R^{x} and R^{y} are each separately selected from hydrogen, C₁₋₆alkylOC(═O)—, C₁₋₆alkyl, C₁₋₆alkylC(═O)—, aryl, arylalkyl, cycloalkyl, and heterocyclyl;
Y¹ is selected from O (oxygen), S (sulfur), S(O), SO₂, NR², and C(R²)₂;
each A¹ is separately selected from the group consisting of C₂₋₆alkenyl, C₁₋₆alkyl, and —(CH₂)ₙ—O—(CH₂)ₘ—, each optionally substituted with one or more R²;
each R² is separately selected, wherein R² is selected from the group consisting of hydrogen, C₁₋₆alkoxy, C₁₋₆alkyl, aryl, halo, hydroxy, R^{a}R^{b}N—, and C₁₋₆alkyl optionally substituted with up to 9 halo, or optionally two vicinal R² and the carbons to which they are attached are together a fused three- to six-membered carbocyclic ring optionally substituted with up to two C₁₋₆alkyl groups, or optionally two geminal R² and the carbon to which they are attached are together a three- to six-membered carbocyclic ring optionally substituted with up to two C₁₋₆alkyl groups;
L⁴ is selected from the group consisting of -(J²)ₛ-(L⁵)ₛ-(J²)ₛ-(L⁵)ₛ-J²-, 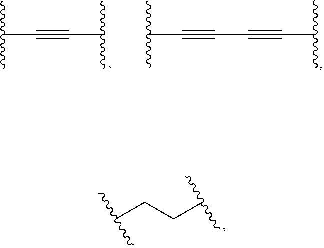
—C(═O)—, O (oxygen), —OC(R²)₂—, 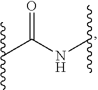
—C(CF₃)₂NR^{2c}—, NH, and —(CH═CH)—;
J² is aryl, heteroaryl, heterocyclyl, cycloalkyl, cycloalkenyl, or polycyclic hydrocarbon, each optionally substituted with one or more R¹⁵;
each R¹⁴ is separately selected from the group consisting of hydroxy, C₁₋₆alkoxy optionally substituted with up to 9 fluoro, C₁₋₆alkylOC₁₋₆alkyl, C₁₋₆alkylOC(═O)—, arylalkylOC(═O)—, —COOH, halo, and C₁₋₆alkyl optionally substituted with up to 9 halo;
each R¹⁵ is separately selected from the group consisting of halo, hydroxy, C₁₋₆alkylOC₁₋₆alkyl, C₁₋₆alkylOC(═O)—, arylalkylOC(═O)—, —COOH, R⁹, R^{x}R^{y}N, R^{x}R^{y}NC(═O), R^{x}R^{y}NC₁₋₆alkyl, heteroaryl, aryl, C₁₋₆alkyl optionally substituted with up to 9 halo, C₁₋₆alkyl substituted with up to 5 hydroxy, C₁₋₆alkoxy optionally substituted with up to 9 halo, C₁₋₆haloalkyl, R^{a}R^{b}N—, (R^{a}R^{b}N)alkyl, (R^{a}R^{b}N)C(═O)—, C₁₋₆alkyl substituted with up to 5 hydroxy, said substituent aryl and heteroaryl are each optionally substituted with one or more R¹⁴, or optionally two vicinal R¹⁵ and the carbons to which they are attached are together a fused three- to six-membered carbocyclic ring optionally substituted with up to two C₁₋₆alkyl groups, or optionally two geminal R¹⁵ and the carbon to which they are attached are together a three- to six-membered carbocyclic ring optionally substituted with up to two C₁₋₆alkyl groups, or optionally two geminal R¹⁵ are together oxo;
each R^{a}R^{b}N is separately selected, wherein R^{a} and R^{b} are each separately selected from the group consisting of hydrogen, C₂₋₆alkenyl, and C₁₋₆alkyl;
each L⁵ is separately selected from the group consisting of 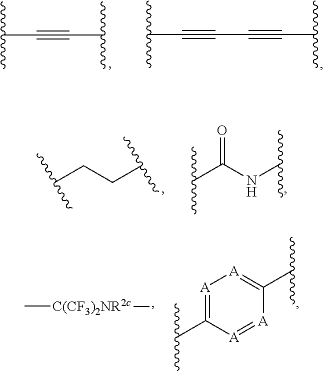
—C(R²)₂, —C(R²)₂O—, —C(═O)—, O (oxygen), NH, and —(CH═CH)—;
L⁶ is selected from the group consisting of 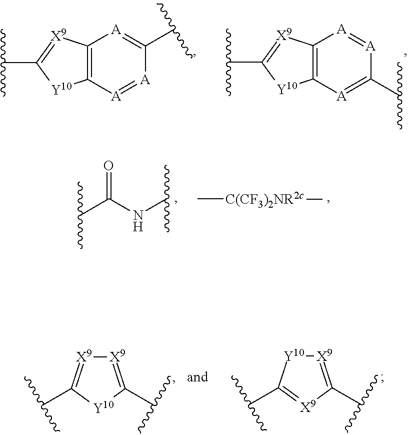
L⁷ is selected from the group consisting of 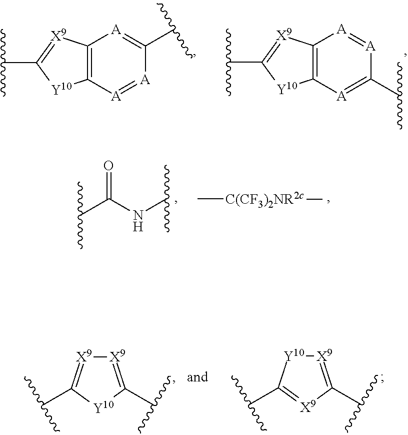
each A is separately selected from the group consisting of CR³ and N (nitrogen);
each R³ is separately selected from the group consisting of hydrogen, C₁₋₆alkoxy, C₁₋₆alkylOC₁₋₆alkyl, C₁₋₆alkylOC(═O)—, arylalkylOC(═O)—, —COOH, halo, hydroxy, R^{a}R^{b}N—, (R^{a}R^{b}N)alkyl, (R^{a}R^{b}N)C(═O)—, and C₁₋₆alkyl optionally substituted with up to 9 halo and up to 5 hydroxy;
R^{2c} is selected from the group consisting of hydrogen, C₁₋₆alkyl, C₂₋₆alkenyl, C₂₋₆alkynyl, C₃₋₇cycloalkyl, aryl, arylalkyl, heteroaryl, heteroarylalkyl, heterocyclyl, and heterocyclylalkyl, said alkyl optionally substituted with R^{e}R^{f}N—, alkoxy, or C₁₋₆alkylS—;
each X⁹ is separately selected from the group consisting of CH and N (nitrogen);
each X¹⁰ is (C(R²)₂)_{q};
each Y¹⁰ is separately selected from the group consisting of —CH₂— and —NH—;
each Y¹¹ is separately selected from the group consisting of −O(C(R²)₂)ₙ—, —S(C(R²)₂)ₙ—, —S(O)(C(R²)₂)ₙ—, —SO₂(C(R²)₂)ₙ—, —NR²(C(R²)₂)ₙ—, and (C(R²)₂)_{q};
each m separately is 1 or 2;
each n separately is 0, 1 or 2;
each p separately is 1, 2, 3 or 4;
each q separately is 1, 2, 3, 4 or 5;
each r separately is 0, 1, 2, 3, or 4;
each s separately is 0 or 1;
Q⁷ is selected from the group consisting of J², 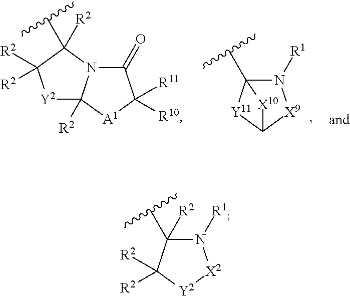
X² is (C(R²)₂)_{q}, 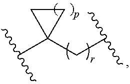
or X₂ is null;
Y² is selected from O (oxygen), S (sulfur), S(O), SO₂, NR², and C(R²)₂;
R⁹ is selected from the group consisting of hydrogen and —C(═O)R^{9a};
R^{9a} is selected from the group consisting of —NR^{9b}R^{9c}, —OR^{9d}, C₁₋₆alkyl optionally substituted with up to 9 halo, and optionally substituted aryl;
R^{9b} is selected from the group consisting of hydrogen, C₁₋₆alkyl optionally substituted with up to 9 halo, and optionally substituted aryl;
R^{9c} is selected from the group consisting of C₁₋₆alkyl optionally substituted with up to 9 halo, and optionally substituted aryl; and
R^{9d} is selected from the group consisting of C₁₋₆alkyl optionally substituted with up to 9 halo, and optionally substituted aryl,
provided that the compound is not selected from the group consisting of:

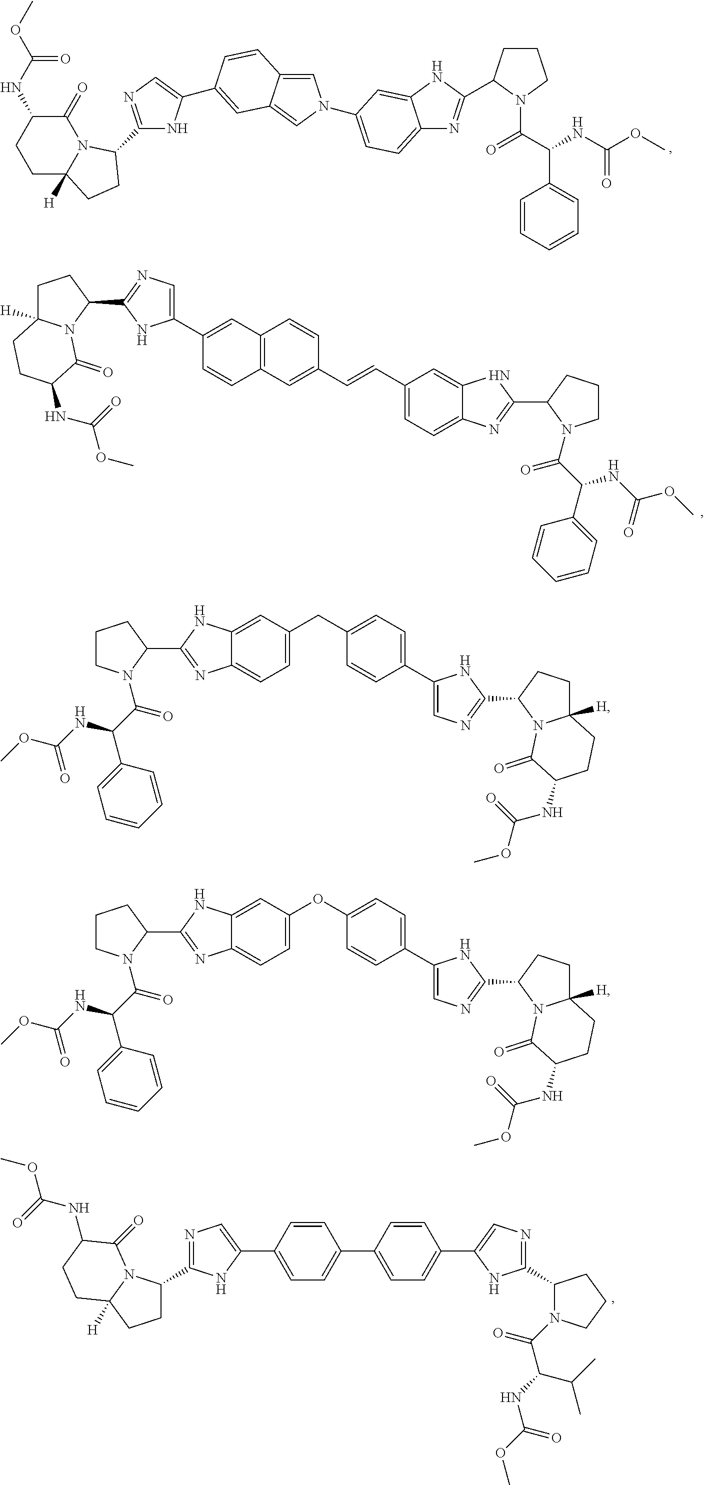

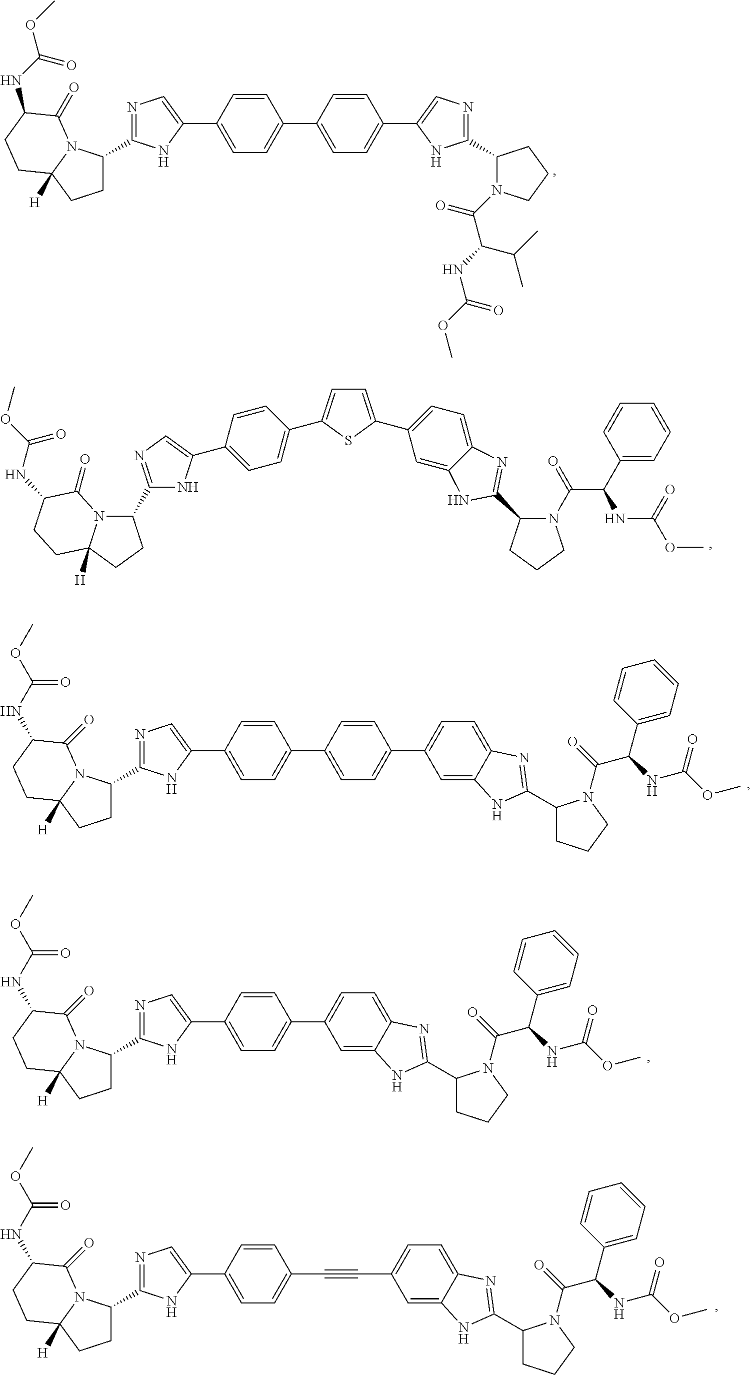

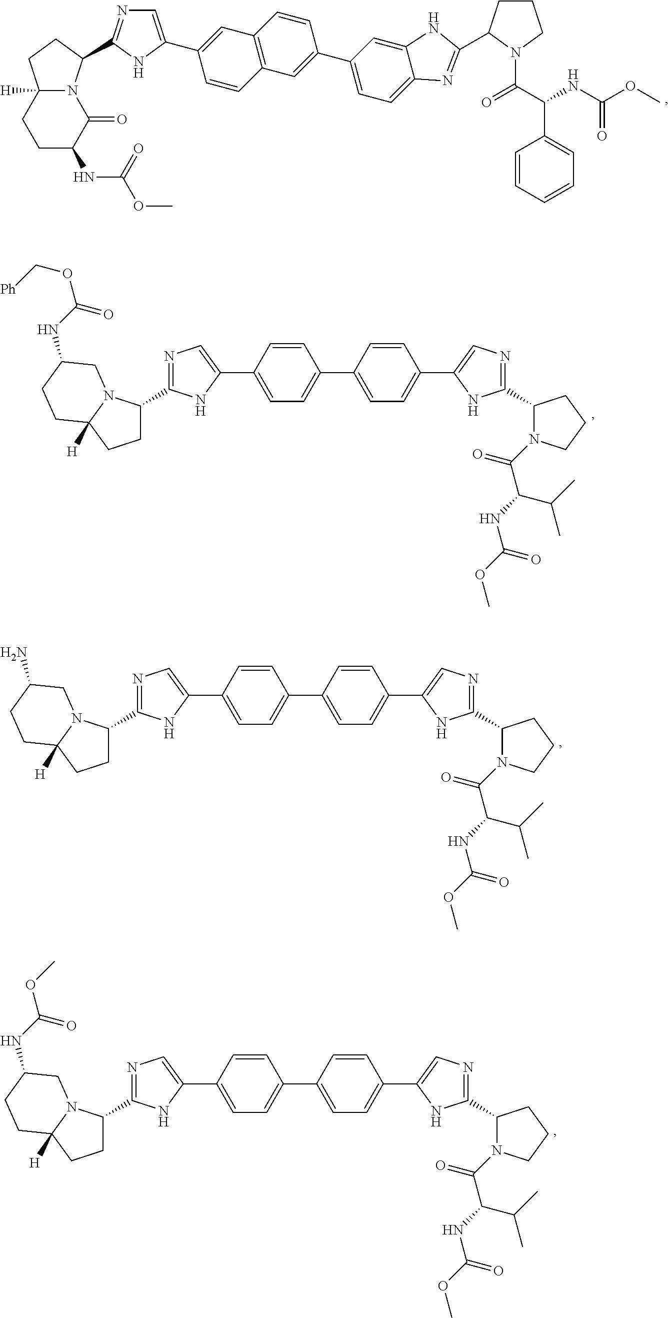

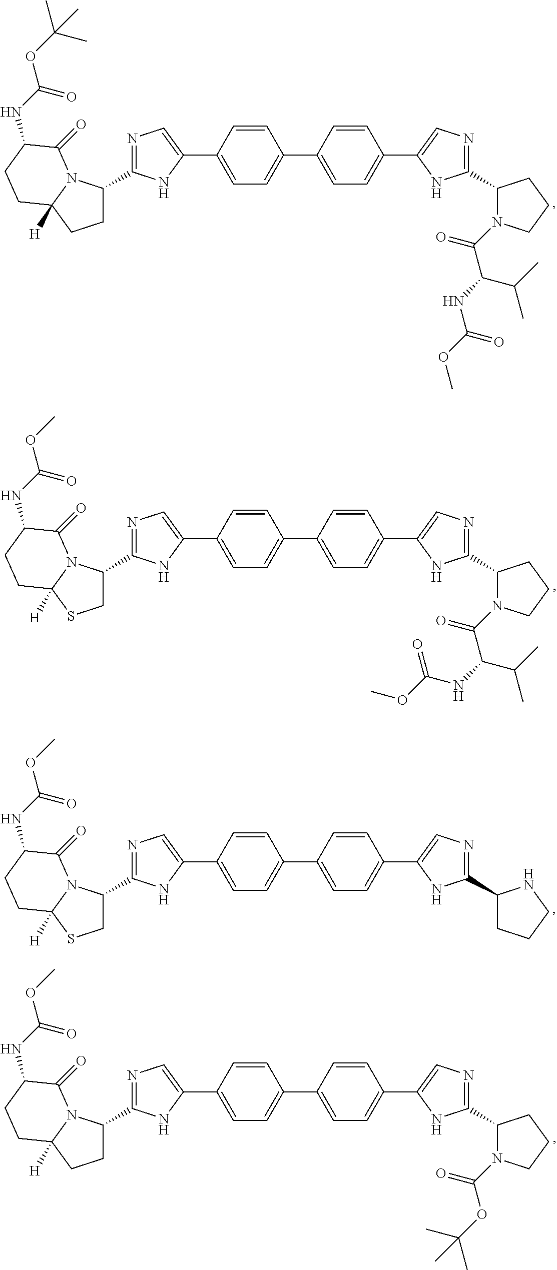

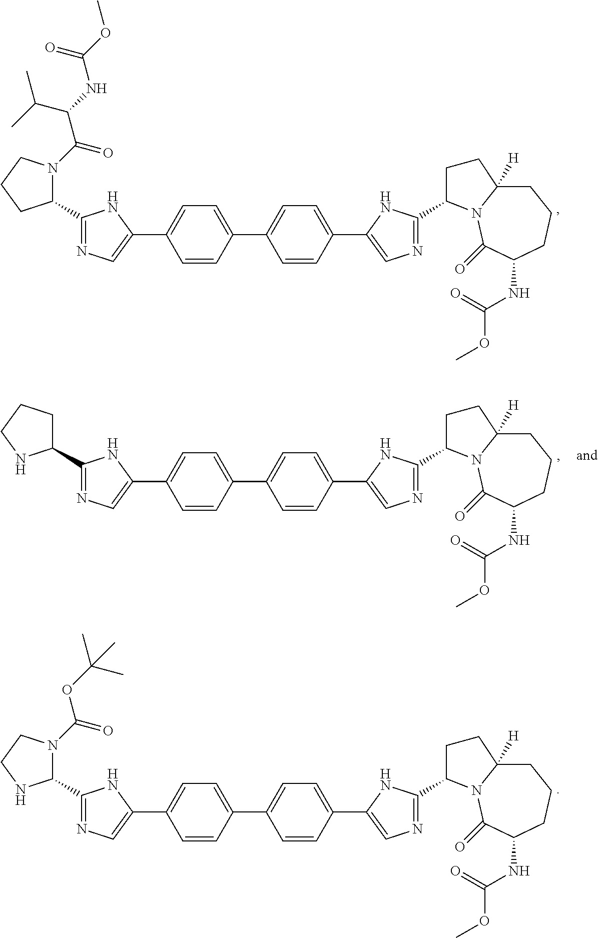

71. The compound of claim 70, wherein
L⁴ is selected from the group consisting of 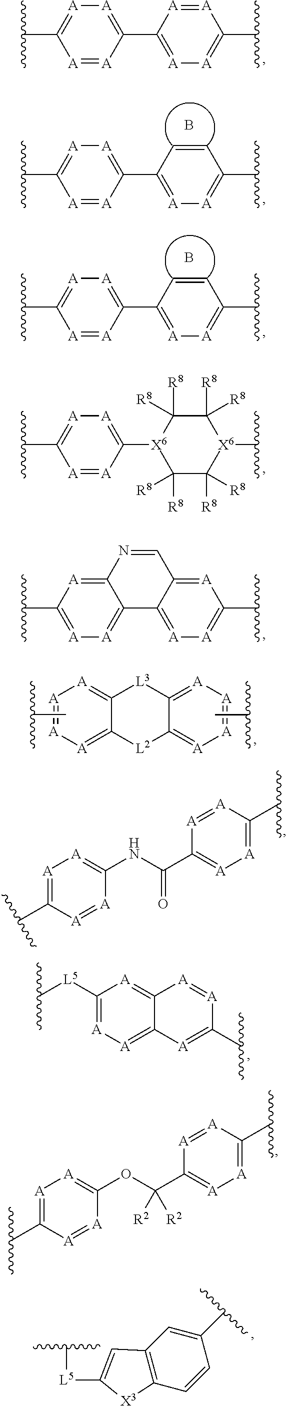 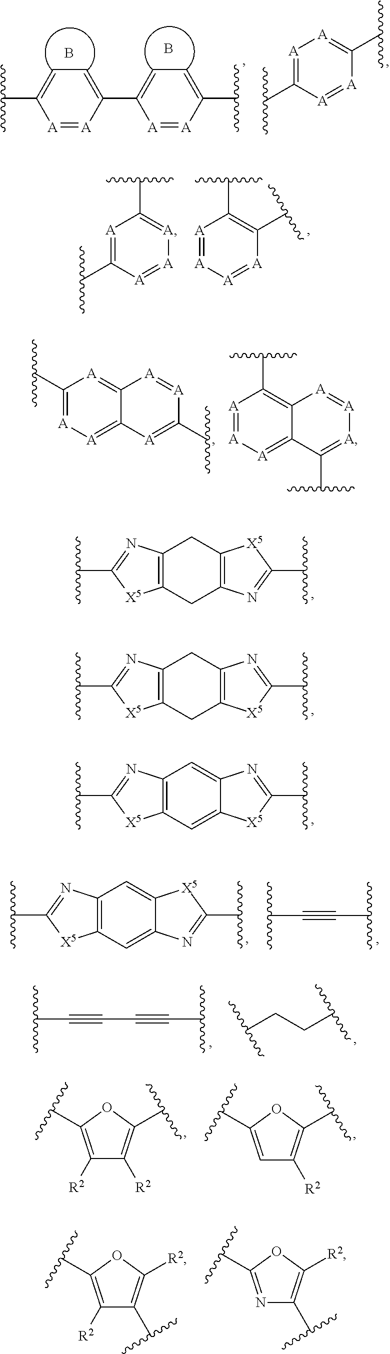 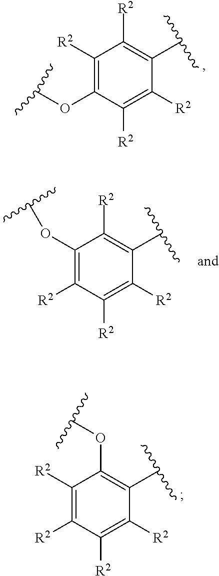
L² is selected from the group consisting of —C(═O)—, —(CH₂CH₂)—, —(CH₂O)—, —(CH₂S)—, —(CH═CH)—, —(CH═N)—, —NH—, O (oxygen), S (sulfur), and —CH₂—;
L³ is selected from the group consisting of 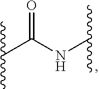
—(NR⁹)—, O (oxygen), S (sulfur), and —CH₂—;
L⁵ is selected from the group consisting of 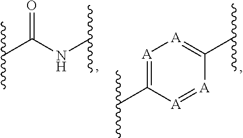
O (oxygen), NH, and —(CH═CH)—;
each X³ is separately selected from the group consisting of NH, O (oxygen), and S (sulfur);
each X⁵ is separately selected from the group consisting of —NH—, O (oxygen), S (sulfur), and —CH₂—;
each X⁶ is separately selected from the group consisting of N (nitrogen), and CR⁸;
each R⁸ is separately selected from the group consisting of hydrogen, C₁₋₆alkylOC₁₋₆alkyl, C₁₋₆alkylOC(═O)—, arylalkylOC(═O)—, halo, (R^{a}R^{b}N)alkyl, (R^{a}R^{b}N)C(═O)—, and C₁₋₆alkyl optionally substituted with up to 9 halo and up to 5 hydroxy;
each B is separately selected, wherein B is a fused optionally substituted saturated or unsaturated three- to seven-membered carbocyclic ring or a fused optionally substituted saturated or unsaturated three- to seven-membered heterocyclic ring, each optionally substituted with one or more R⁴; and
each R⁴ is separately selected from the group consisting of C₁₋₆alkoxy, C₁₋₆alkylOC₁₋₆alkyl, C₁₋₆alkylOC(═O)—, arylalkylOC(═O)—, —COOH, halo, C₁₋₆haloalkyl, hydroxy, R^{a}R^{b}N—, (R^{a}R^{b}N)alkyl, (R^{a}R^{b}N)C(═O)—, and C₁₋₆alkyl optionally substituted with up to 9 halo and up to 5 hydroxy, or optionally two geminal R⁴ are together oxo.

72. The compound of claim 70, wherein Q⁷ is: 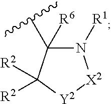
and
R⁶ is C₁₋₆alkyl optionally substituted with up to 9 halo.

73. The compound of claim 72, wherein R⁶ is methyl.

74. The compound of claim 70, wherein
L⁴ is selected from the group consisting of O (oxygen), —OC(R²)₂—, 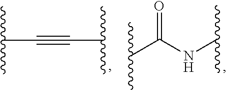
—C(CF₃)₂NR^{2c}—, 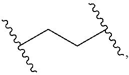
NH, and —(CH═CH)—, Y⁶, Y⁶—Y⁶, Y⁶—Y⁶—Y⁶;
each Y⁶ is separately selected from the group consisting of aryl, heteroaryl, heterocyclyl, polycyclic hydrocarbon, each optionally substituted with one or more substituents selected from the groups consisting of R², R³, R⁴, and R⁸;
each R² is separately selected, wherein R² is selected from the group consisting of C₁₋₆alkoxy, C₁₋₆alkyl, aryl, halo, hydroxy, R^{a}R^{b}N—, and C₁₋₆alkyl optionally substituted with up to 9 halo, or optionally two vicinal R² and the carbons to which they are attached are together a fused three- to six-membered carbocyclic ring optionally substituted with up to two C₁₋₆alkyl groups, or optionally two geminal R² and the carbon to which they are attached are together a three- to six-membered carbocyclic ring optionally substituted with up to two C₁₋₆alkyl groups;
each R³ is separately selected from the group consisting of C₁₋₆alkoxy, C₁₋₆alkylOC₁₋₆alkyl, C₁₋₆alkylOC(═O)—, arylalkylOC(═O)—, —COOH, halo, hydroxy, R^{a}R^{b}N—, (R^{a}R^{b}N)alkyl, (R^{a}R^{b}N)C(═O)—, and C₁₋₆alkyl optionally substituted with up to 9 halo and up to 5 hydroxy;
each R⁴ is separately selected from the group consisting of C₁₋₆alkoxy, C₁₋₆alkylOC₁₋₆alkyl, C₁₋₆alkylOC(═O)—, arylalkylOC(═O)—, —COOH, halo, C₁₋₆haloalkyl, hydroxy, R^{a}R^{b}N—, (R^{a}R^{b}N)alkyl, (R^{a}R^{b}N)C(═O)—, and C₁₋₆alkyl optionally substituted with up to 9 halo and up to 5 hydroxy, or optionally two geminal R⁴ are together oxo; and
each R⁸ is separately selected from the group consisting of C₁₋₆alkylOC₁₋₆alkyl, C₁₋₆alkylOC(═O)—, arylalkylOC(═O)—, halo, (R^{a}R^{b}N)alkyl, (R^{a}R^{b}N)C(═O)—, and C₁₋₆alkyl optionally substituted with up to 9 halo and up to 5 hydroxy.

75. The compound of claim 70, having the structure: 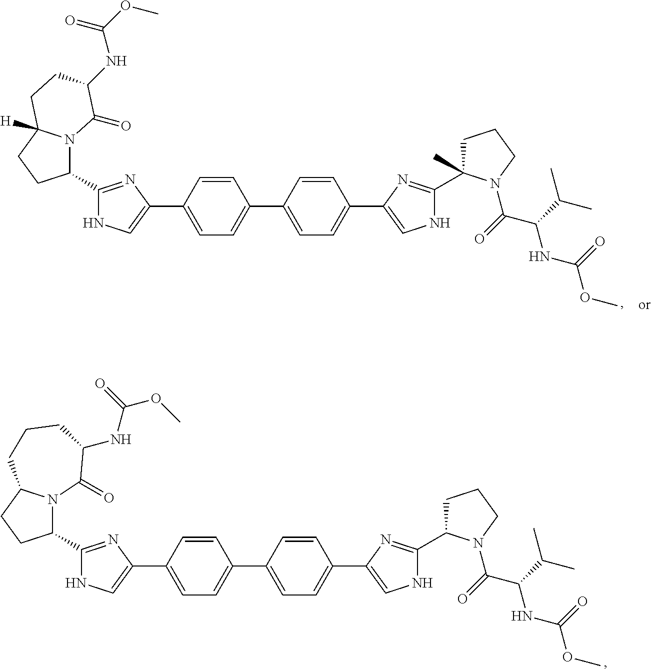
or a pharmaceutically acceptable salt thereof.

76. A compound having the structure of Formula XIV: 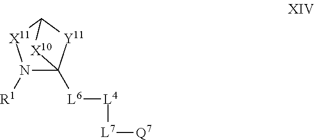
or a pharmaceutically acceptable salt thereof,
wherein:
Q⁷ is selected from the group consisting of J², 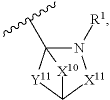
and 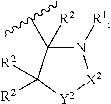
each X¹⁰ is (C(R²)₂)_{q};
each X¹¹ is separately selected from the group consisting of (C(R²)₂)_{q}, and 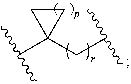
each Y¹¹ is separately selected from the group consisting of —O(C(R²)₂)ₙ—, —S(C(R²)₂)ₙ—, —S(O)(C(R²)₂)ₙ—, —SO₂(C(R²)₂)ₙ—, —NR²(C(R²)₂)ₙ—, and (C(R²)₂)_{q};
each R¹ is separately selected from the group consisting of hydrogen, R^{1a}C(═O)— and R^{1a}C(═S)—;
each R^{1a} is separately selected from the group consisting of —C(R^{2a})₂NR^{3a}R^{3b}, alkoxyalkyl, C₁₋₆alkylOC(═O)—, C₁₋₆alkylOC(═O)C₁₋₆alkyl, C₁₋₆alkylC(═O)C₁₋₆alkyl, aryl, aryl(CH₂)ₙ—, aryl(CH₂)ₙO—, aryl(CH═CH)ₘ—, arylalkylO—, arylalkyl, arylOalkyl, cycloalkyl, (cycloalkyl)(CH═CH)ₘ—, (cycloalkyl)alkyl, cycloalkylOalkyl, heterocyclyl, heterocyclyl(CH═CH)ₘ—, heterocyclylalkoxy, heterocyclylalkyl, heterocyclylOalkyl, hydroxyalkyl, R^{c}R^{d}N—, R^{c}R^{d}N(CH₂)ₙ—, (R^{c}R^{d}N)(CH═CH)ₘ—, (R^{c}R^{d}N)alkyl, (R^{c}R^{d}N)C(═O)—, C₁₋₆alkoxy optionally substituted with up to 9 halo, and C₁₋₆alkyl optionally substituted with up to 9 halo, said aryl and heteroaryl each optionally substituted with cyano, halo, nitro, hydroxyl, C₁₋₆alkoxy optionally substituted with up to 9 halo, and C₁₋₆alkyl optionally substituted with up to 9 halo;
each R^{c}R^{d}N is separately selected, wherein R^{c} and R^{d} are each separately selected from hydrogen, alkoxyC(═O)—, C₁₋₆alkyl, C₁₋₆alkylC(═O)—, C₁₋₆alkylsulfonyl, arylalkylOC(═O)—, arylalkyl, arylalkylC(═O)—, arylC(═O)—, arylsulfonyl, heterocyclylalkyl, heterocyclylalkylC(═O)—, heterocyclylC(═O)—, (R^{e}R^{f}N)alkyl, (R^{e}R^{f}N)alkylC(═O)—, and (R^{e}R^{f}N)C(═O)—, wherein the alkyl part of arylalkyl, arylalkylC(═O)—, heterocyclylalkyl, and heterocyclylalkylC(═O)— are each optionally substituted with one R^{e}R^{f}N— group; and wherein the aryl part of arylalkyl, arylalkylC(═O)—, arylC(═O)—, and arylsulfonyl, and the heterocyclyl part of heterocyclylalkyl, heterocyclylalkylC(═O)—, and heterocyclylC(═O)— are each optionally substituted with up to three substituents each independently selected from the group consisting of cyano, halo, nitro, C₁₋₆alkoxy optionally substituted with up to 9 halo, and C₁₋₆alkyl optionally substituted with up to 9 halo;
each R^{e}R^{f}N is separately selected, wherein R^{e} and R^{f} are each separately selected from hydrogen, C₁₋₆alkyl, aryl, arylalkyl, cycloalkyl, (cyclolalkyl)alkyl, heterocyclyl, heterocyclylalkyl, (R^{x}R^{y}N)alkyl, and (R^{x}R^{y}N)C(═O)—;
each R^{x}R^{y}N is separately selected, wherein R^{x} and R^{y} are each separately selected from hydrogen, C₁₋₆alkylOC(═O)—, C₁₋₆alkyl, C₁₋₆alkylC(═O)—, aryl, arylalkyl, cycloalkyl, and heterocyclyl;
each C(R^{2a})₂ is separately selected, wherein each R^{2a} is separately selected from the group consisting of hydrogen, C₁₋₆alkyl optionally substituted with up to 9 halo, aryl(CH₂)ₙ—, and heteroaryl(CH₂)ₙ—, said aryl and heteroaryl each optionally substituted with cyano, halo, nitro, hydroxyl, C₁₋₆alkoxy optionally substituted with up to 9 halo, and C₁₋₆alkyl optionally substituted with up to 9 halo, or C(R^{2a})₂ is 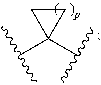
each R^{3a} is separately selected from the group consisting of hydrogen, and optionally substituted C₁₋₆alkyl;
each R^{3b} is separately selected from the group consisting of optionally substituted C₁₋₆alkyl, heteroaryl, —(CH₂)ₙC(═O)NR^{4a}R^{4b}, —(CH₂)ₙC(═O)OR^{5a}, and —(CH₂)ₙC(═O)R^{6a} said heteroaryl optionally substituted with cyano, halo, nitro, hydroxyl, C₁₋₆alkoxy optionally substituted with up to 9 halo, and C₁₋₆alkyl optionally substituted with up to 9 halo;
each R^{4a}R^{4b}N is separately selected, wherein R^{4a} and R^{4b} are each separately selected from the group consisting of hydrogen, optionally substituted C₁₋₆alkyl, and aryl(CH₂)ₙ—;
each R^{5a} is separately selected from the group consisting of optionally substituted C₁₋₆alkyl, and aryl(CH₂)ₙ—;
each R^{6a} is separately selected from the group consisting of optionally substituted C₁₋₆alkyl, and aryl(CH₂)ₙ—;
X¹ is (C(R²)₂)_{q}, 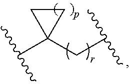
or X¹ is null;
Y¹ is selected from O (oxygen), S (sulfur), S(O), SO₂, NR², and C(R²)₂ with the proviso that when X¹ is null Y¹ is C(R²)₂;
X² is (C(R²)₂)_{q}, 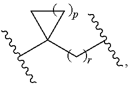
or X² is null;
Y² is selected from O (oxygen), S (sulfur), S(O), SO₂, NR², and C(R²)₂ with the proviso that when X² is null Y² is C(R²)₂;
each R² is separately selected, wherein R² is selected from the group consisting of hydrogen, C₁₋₆alkoxy, C₁₋₆alkyl, aryl, halo, hydroxy, R^{a}R^{b}N—, and C₁₋₆alkyl optionally substituted with up to 9 halo, or optionally two vicinal R² and the carbons to which they are attached are together a fused three- to six-membered carbocyclic ring optionally substituted with up to two C₁₋₆alkyl groups;
each R^{a}R^{b}N is separately selected, wherein R^{a} and R^{b} are each separately selected from the group consisting of hydrogen, C₂₋₆alkenyl, and C₁₋₆alkyl;
L⁴ is selected from the group consisting of -(J²)ₛ-(L⁵)ₛ-(J²)ₛ-(L⁵)ₛ-J²-, 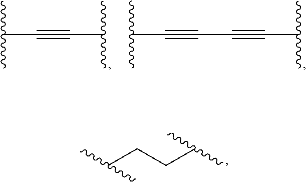
—C(═O)—, O (oxygen), —OC(R²)₂—, 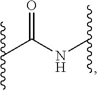
—C(CF₃)₂NR^{2c}—, NH, and —(CH═CH)—;
J² is aryl, heteroaryl, heterocyclyl, cycloalkyl, cycloalkenyl, or polycyclic hydrocarbon, each optionally substituted with one or more R¹⁵;
each R¹⁴ is separately selected from the group consisting of hydroxy, C₁₋₆alkoxy optionally substituted with up to 9 fluoro, C₁₋₆alkylOC₁₋₆alkyl, C₁₋₆alkylOC(═O)—, arylalkylOC(═O)—, —COOH, halo, and C₁₋₆alkyl optionally substituted with up to 9 halo;
each R¹⁵ is separately selected from the group consisting of halo, hydroxy, C₁₋₆alkylOC₁₋₆alkyl, C₁₋₆alkylOC(═O)—, arylalkylOC(═O)—, —COOH, R⁹, R^{x}R^{y}N, R^{x}R^{y}NC(═O), R^{x}R^{y}NC₁₋₆alkyl, heteroaryl, aryl, C₁₋₆alkyl optionally substituted with up to 9 halo, C₁₋₆alkyl substituted with up to 5 hydroxy, C₁₋₆alkoxy optionally substituted with up to 9 halo, C₁₋₆haloalkyl, R^{a}R^{b}N—, (R^{a}R^{b}N)alkyl, (R^{a}R^{b}N)C(═O)—, and C₁₋₆alkyl substituted with up to 5 hydroxy, said substituent aryl and heteroaryl are each optionally substituted with one or more R¹⁴, or optionally two vicinal R¹⁵ and the carbons to which they are attached are together a fused three- to six-membered carbocyclic ring optionally substituted with up to two C₁₋₆alkyl groups, or optionally two geminal R¹⁵ and the carbon to which they are attached are together a three- to six-membered carbocyclic ring optionally substituted with up to two C₁₋₆alkyl groups, or optionally two geminal R¹⁵ are together oxo;
each L⁵ is separately selected from the group consisting of 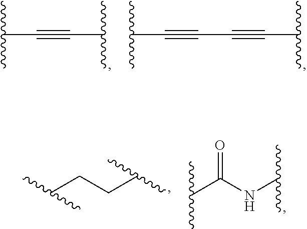
—C(CF₃)₂NR^{2c}—, 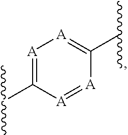
—C(R²)₂—, —C(R²)₂O—, —C(═O)—, O (oxygen), NH, and —(CH═CH)—;
each A is separately selected from the group consisting of CR³ and N (nitrogen);
R^{2c} is selected from the group consisting of hydrogen, C₁₋₆alkyl, C₂₋₆alkenyl, C₂₋₆alkynyl, C₃₋₇cycloalkyl, aryl, arylalkyl, heteroaryl, heteroarylalkyl, heterocyclyl, and heterocyclylalkyl, said alkyl optionally substituted with R^{e}R^{f}N—, alkoxy, or C₁₋₆alkylS—;
L⁶ is selected from the group consisting of 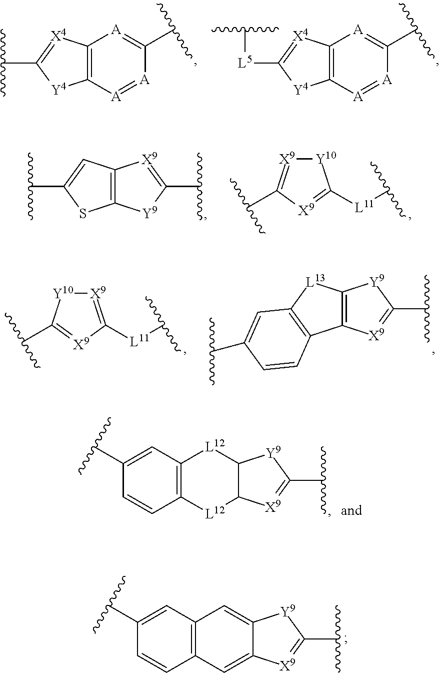
L⁷ is selected from the group consisting of 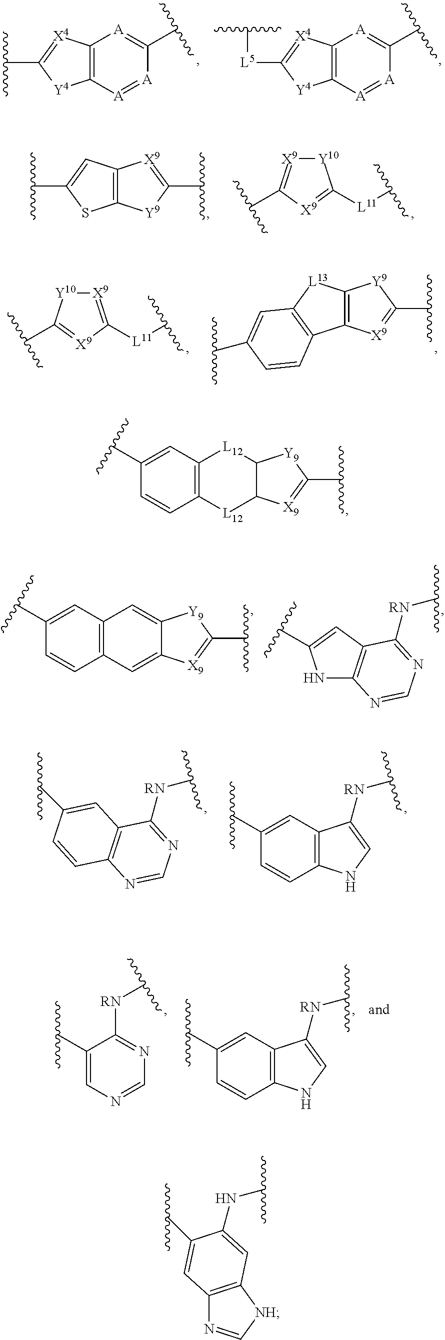
each R is separately selected from the group consisting of hydrogen, C₁₋₆alkylOC(═O)—, arylalkylOC(═O)—, (R^{a}R^{b}N)C(═O)—, and C₁₋₆alkyl optionally substituted with up to 9 halo;
each X⁴ is separately selected from the group consisting of CR⁴ and N (nitrogen), wherein if X⁴ is N (nitrogen) then Y⁴ is not NH;
each Y⁴ is separately selected from the group consisting of C(R⁴)₂, NR⁴, O (oxygen), and S (sulfur);
each Y⁹ is separately selected from the group consisting of —NH—, O (oxygen), and S (sulfur);
each X⁹ is separately selected from the group consisting of CH and N (nitrogen), wherein if X⁹ is N (nitrogen) then Y⁹ is not NH;
each Y¹⁰ is separately selected from the group consisting of —CH₂— and —NH—;
each L¹¹ is separately selected from the group consisting of 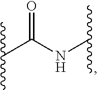
and NH;
each L¹² is separately selected from the group consisting of —CH₂— and —CH₂CH₂—;
each L¹³ is separately selected from the group consisting of —CH₂—, —N═CH—, —CH═CH—, —CH₂CH₂—, —(CH₂)ₘNR⁴(CH₂)ₙ— and —(CH₂)ₘO(CH₂)ₙ—;
each m separately is 1 or 2;
each n separately is 0, 1 or 2;
each p separately is 1, 2, 3 or 4;
each q separately is 1, 2, 3, 4 or 5;
each r separately is 0, 1, 2, 3, or 4;
each s separately is 0 or 1;
each R³ is separately selected from the group consisting of hydrogen, C₁₋₆alkoxy, C₁₋₆alkylOC₁₋₆alkyl, C₁₋₆alkylOC(═O)—, arylalkylOC(═O)—, —COOH, halo, hydroxy, R^{a}R^{b}N—, (R^{a}R^{b}N)alkyl, (R^{a}R^{b}N)C(═O)—, and C₁₋₆alkyl optionally substituted with up to 9 halo and up to 5 hydroxy; and
each R⁴ is separately selected from the group consisting of H (hydrogen), C₁₋₆alkoxy, C₁₋₆alkylOC₁₋₆alkyl, C₁₋₆alkylOC(═O)—, arylalkylOC(═O)—, —COOH, halo, C₁₋₆haloalkyl, hydroxy, R^{a}R^{b}N—, (R^{a}R^{b}N)alkyl, (R^{a}R^{b}N)C(═O)—, and C₁₋₆alkyl optionally substituted with up to 9 halo and up to 5 hydroxy, or optionally two geminal R⁴ are together oxo;
R⁹ is selected from the group consisting of hydrogen and —C(═O)R^{9a};
R^{9a} is selected from the group consisting of —NR^{9b}R^{9c}, —OR^{9d}, C₁₋₆alkyl optionally substituted with up to 9 halo, and optionally substituted aryl;
R^{9b} is selected from the group consisting of hydrogen, C₁₋₆alkyl optionally substituted with up to 9 halo, and optionally substituted aryl;
R^{9c} is selected from the group consisting of C₁₋₆alkyl optionally substituted with up to 9 halo, and optionally substituted aryl; and
R^{9d} is selected from the group consisting of C₁₋₆alkyl optionally substituted with up to 9 halo, and optionally substituted aryl.

77. The compound of claim 76, having the structure: 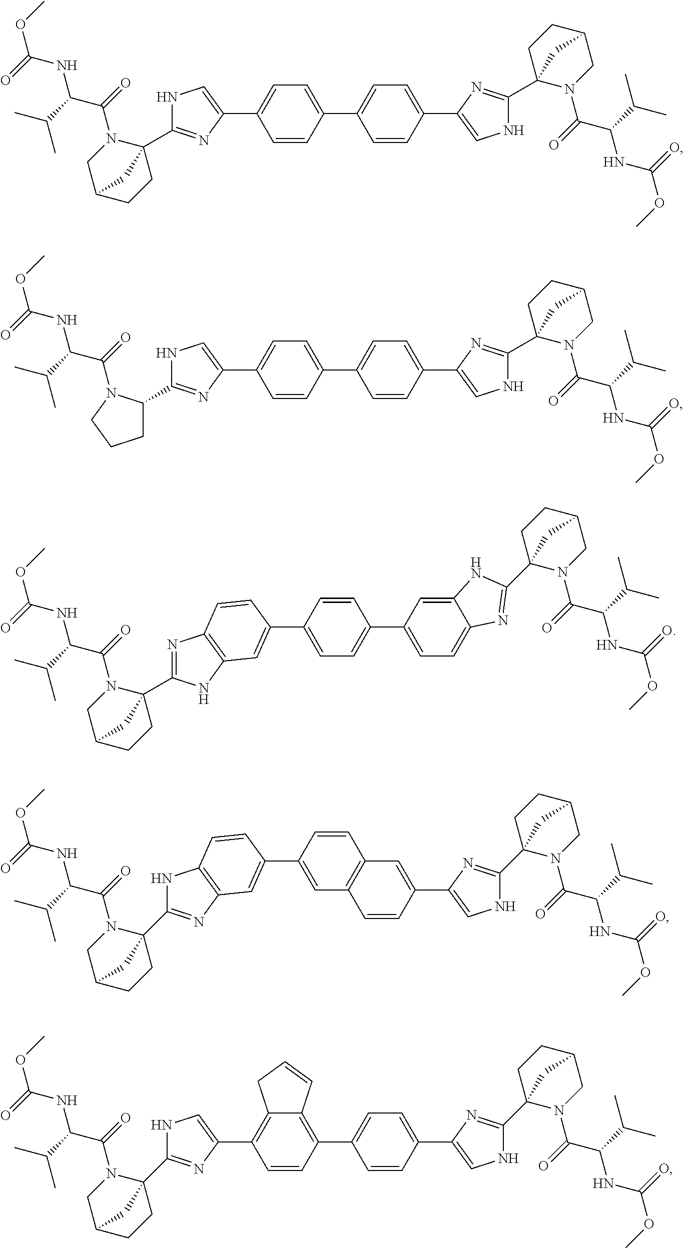 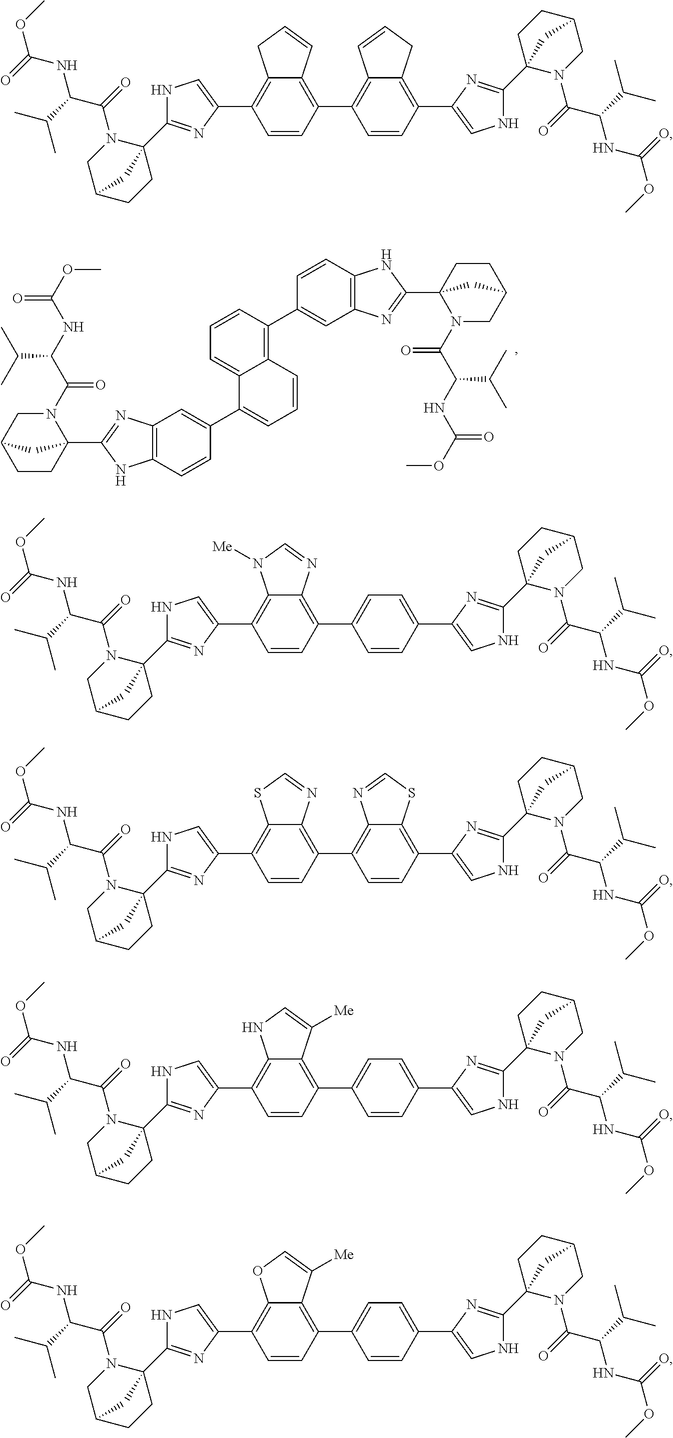 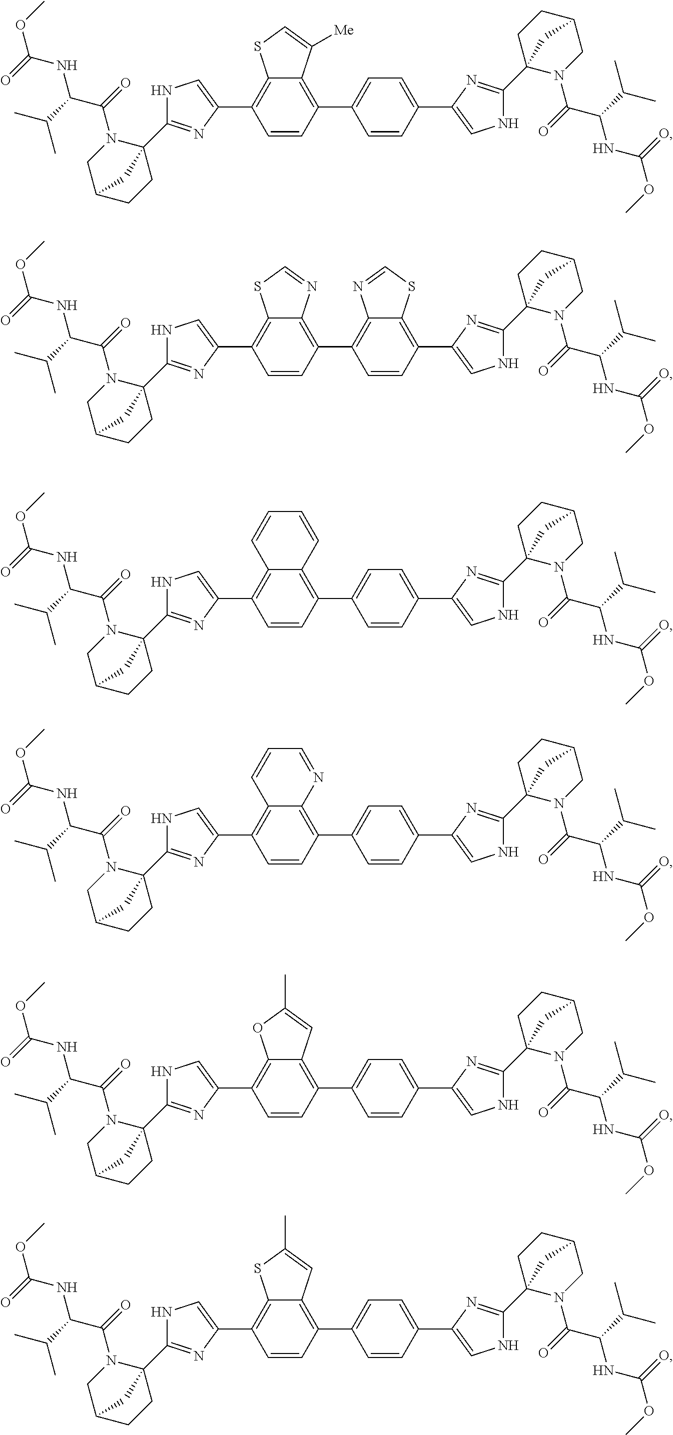 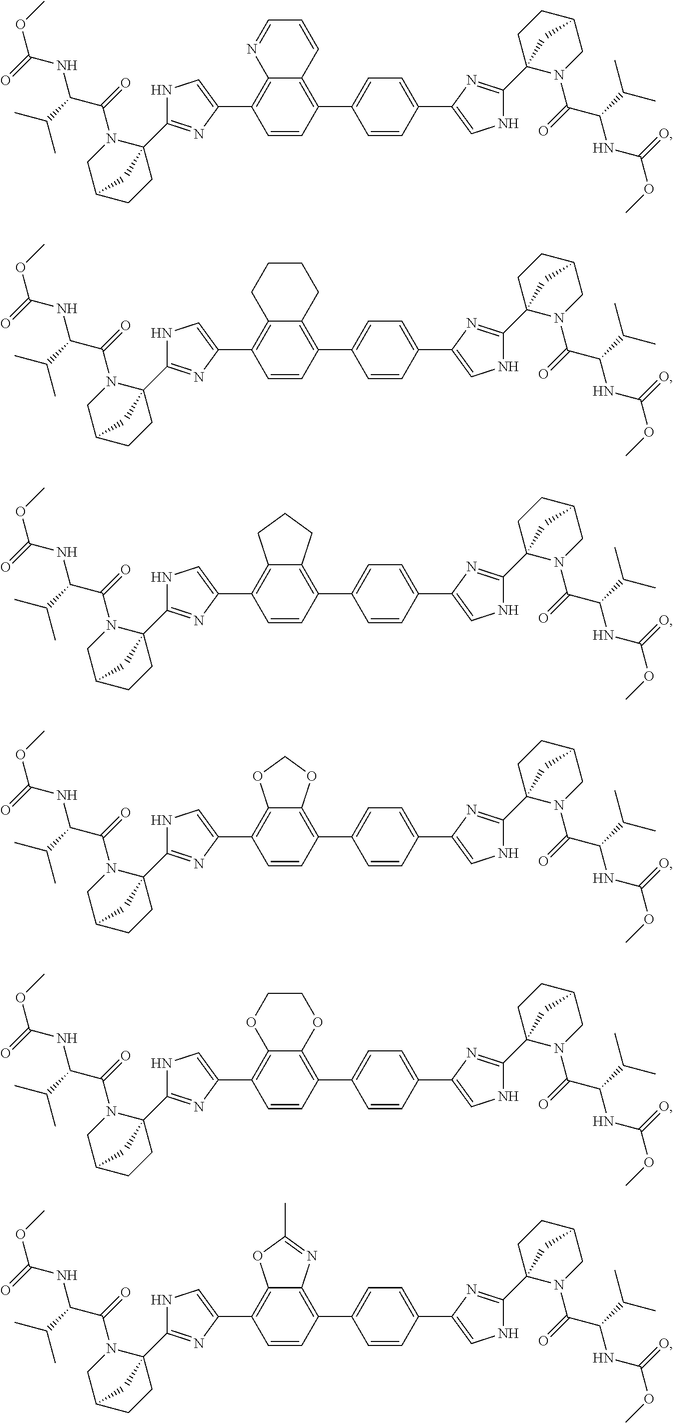 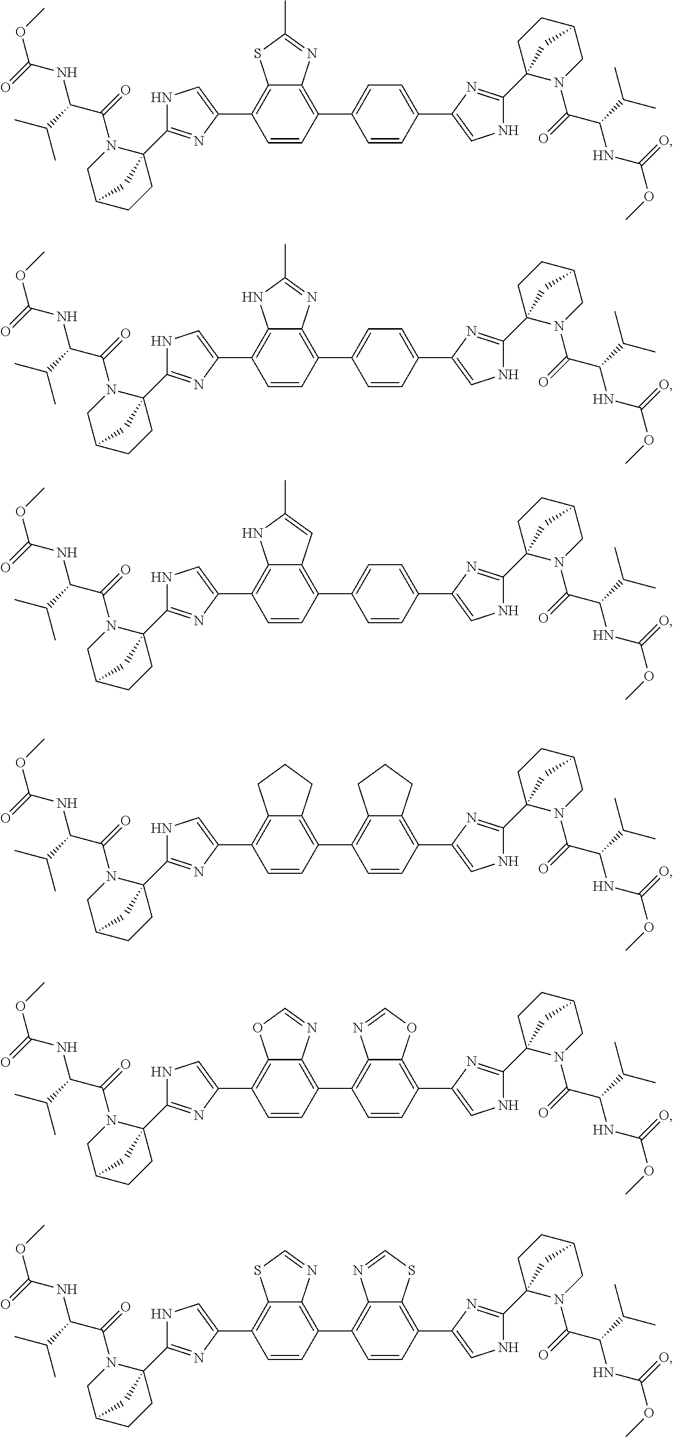 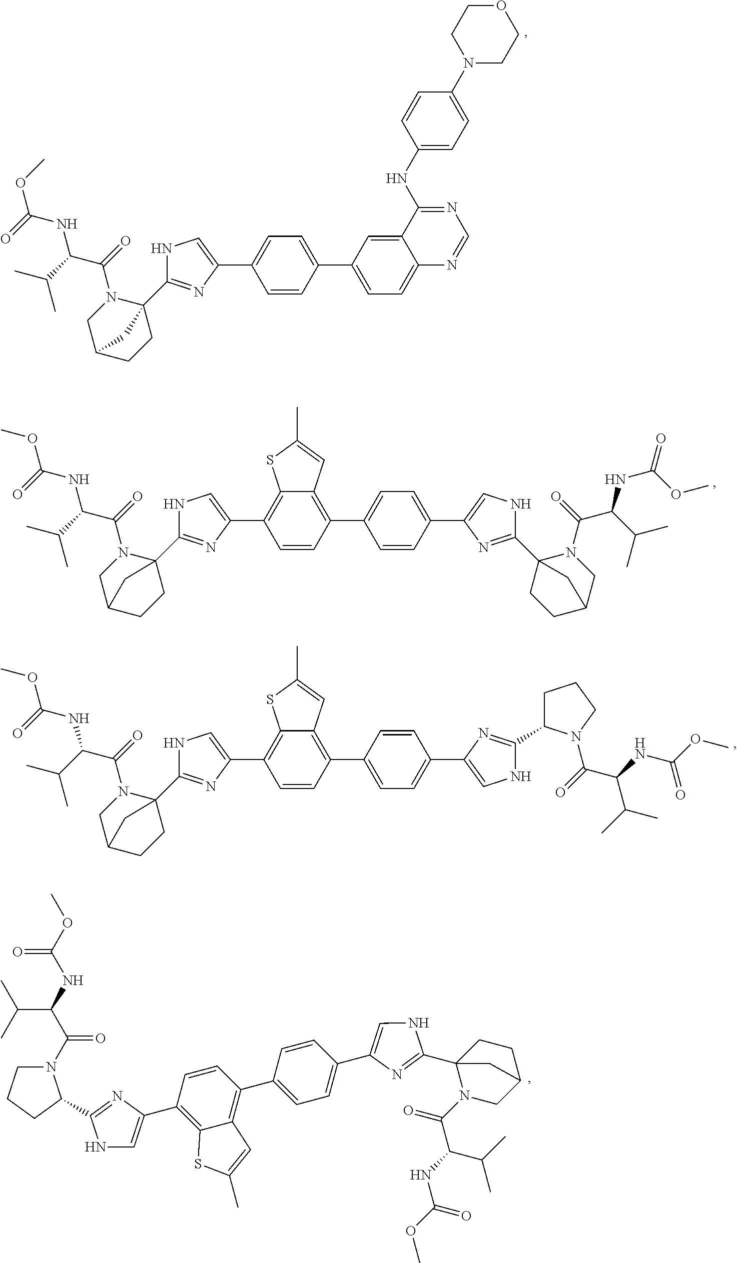 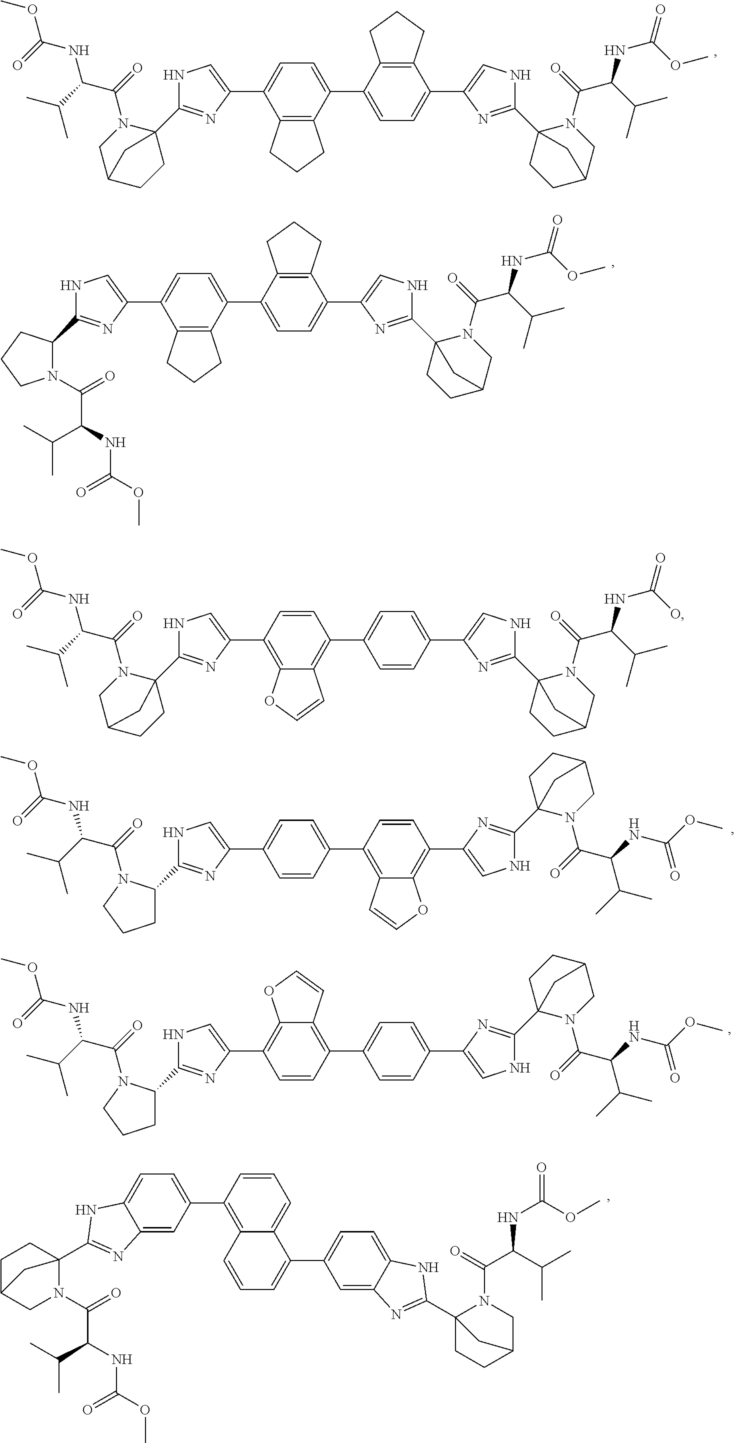 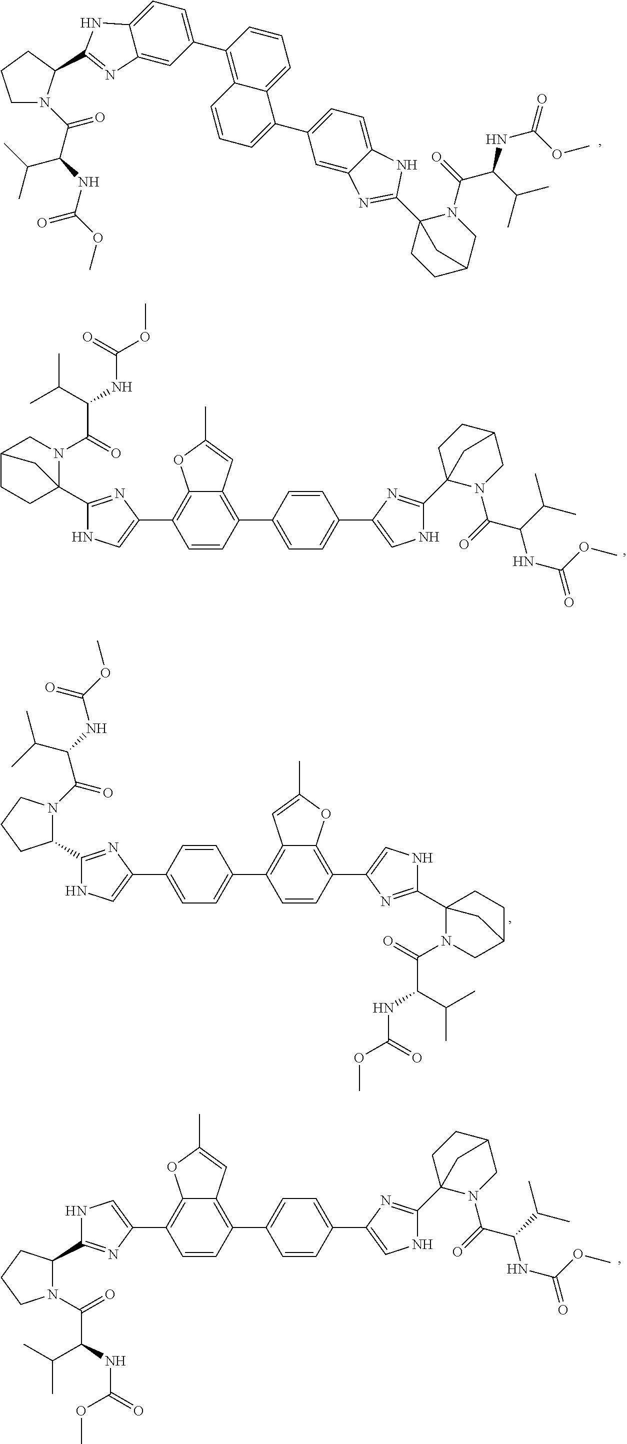 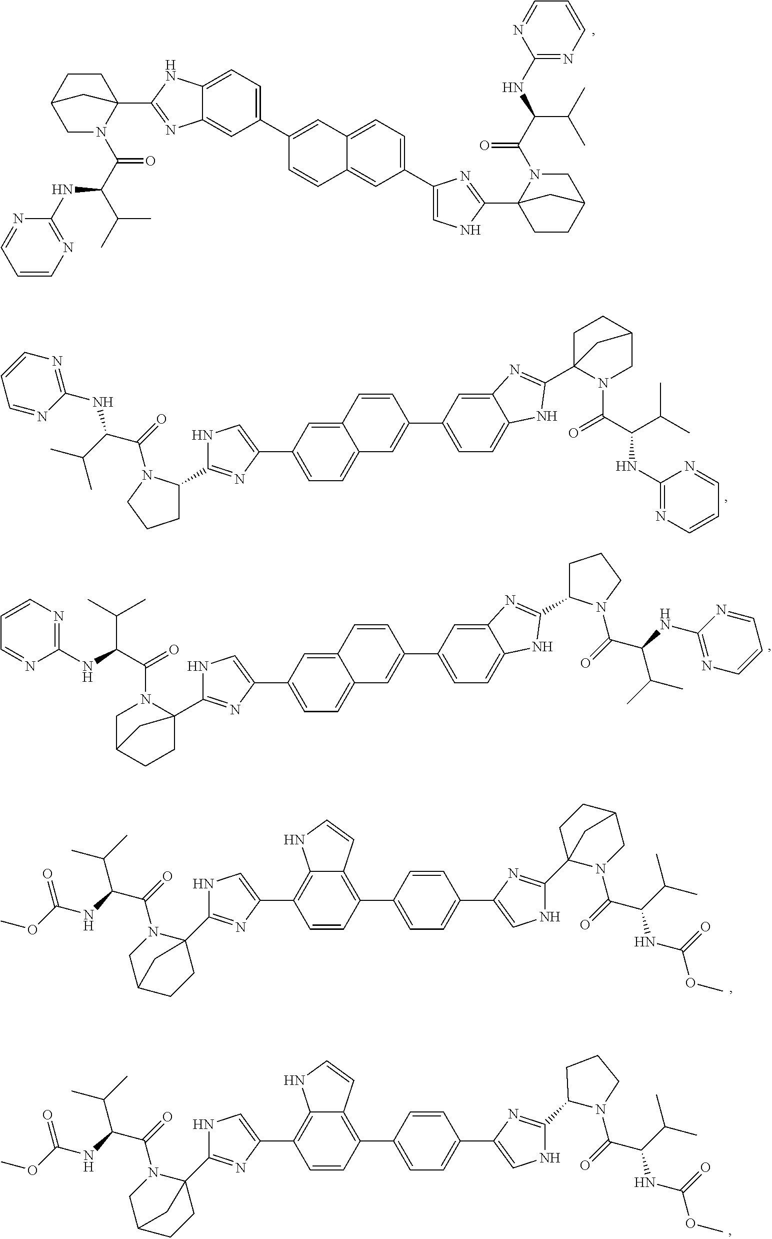 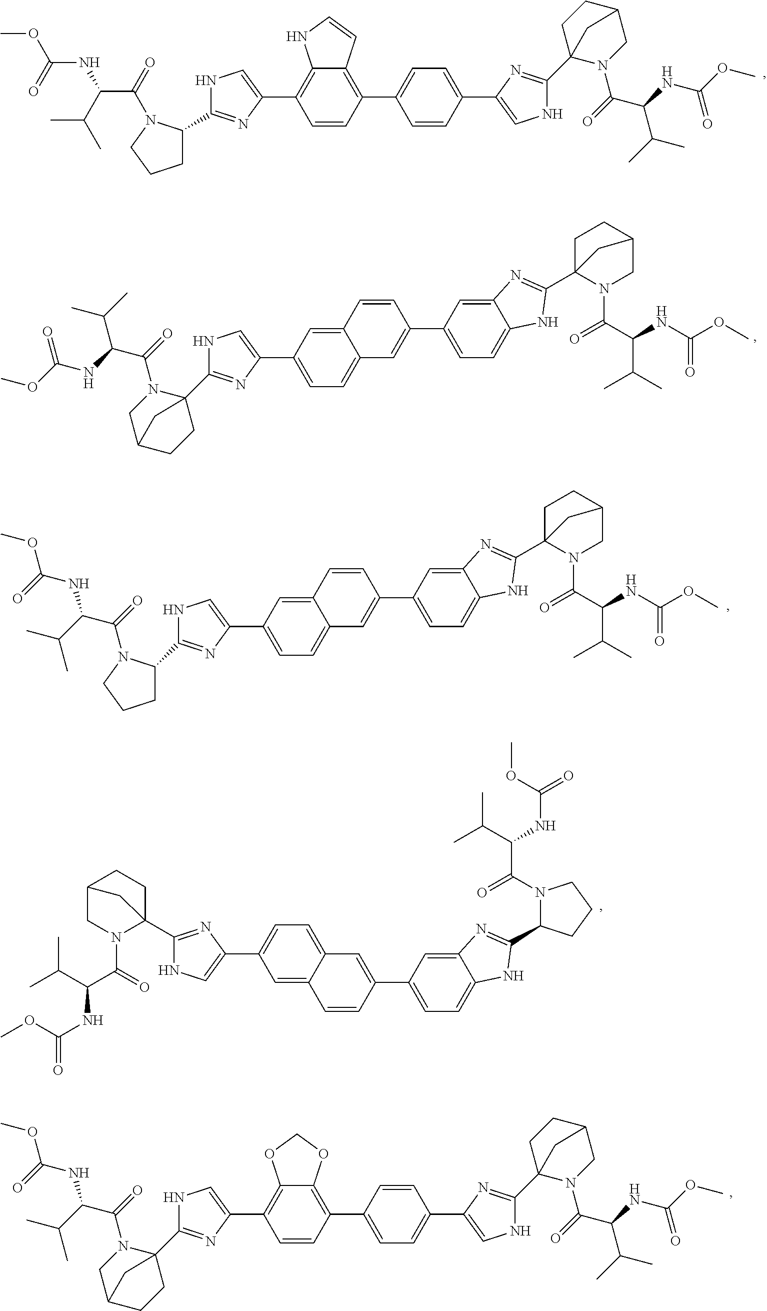 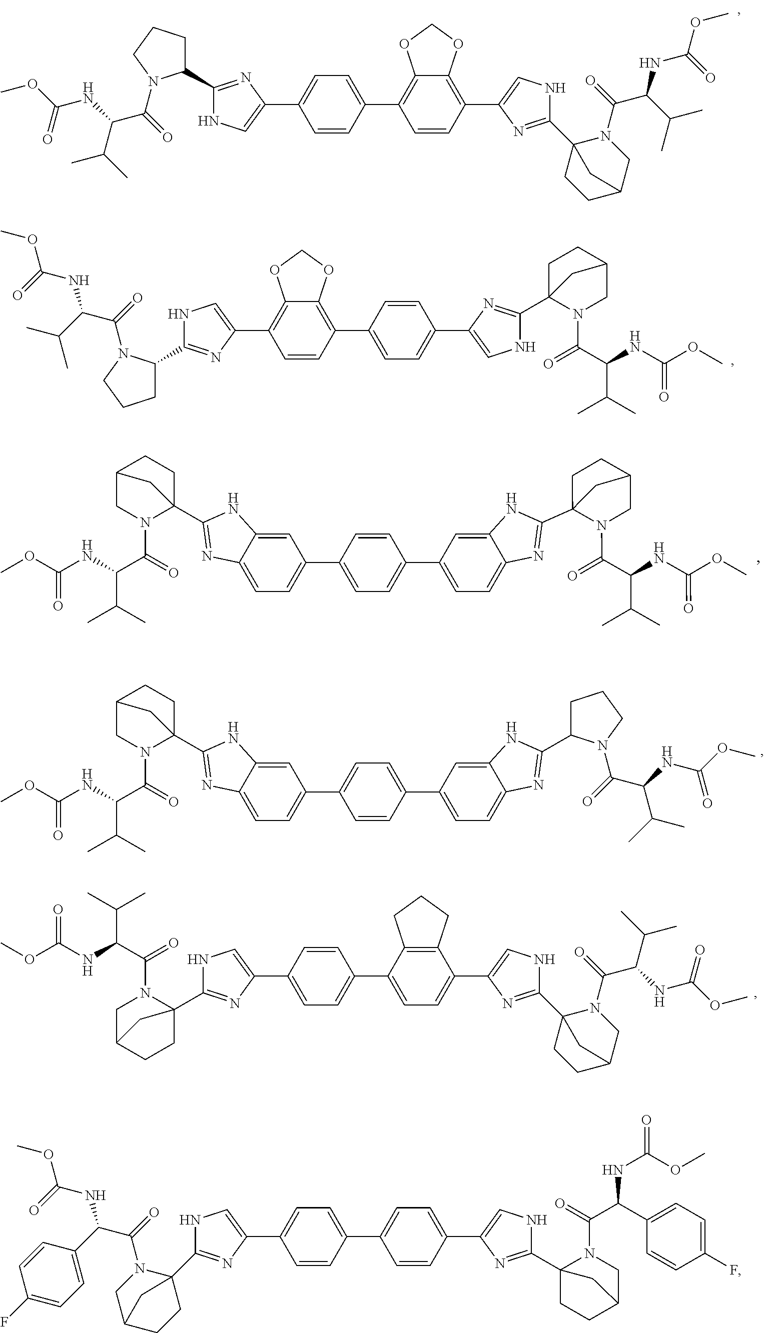 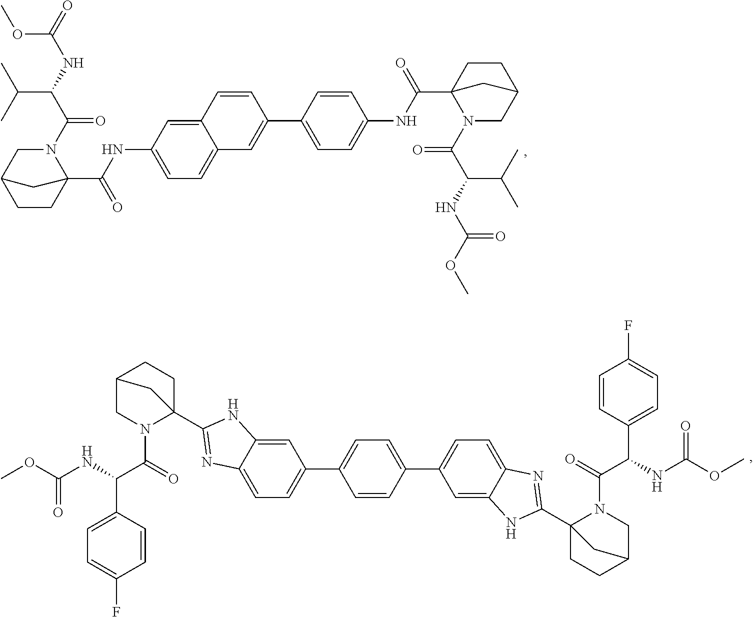
or a pharmaceutically acceptable salt thereof.

78. The compound of claim 76, wherein Q⁷ is: 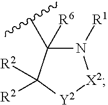
and
R⁶ is C₁₋₆alkyl optionally substituted with up to 9 halo.

79. The compound of claim 78, wherein R⁶ is methyl.

80. The compound of claim 76, wherein
L⁴ is selected from the group consisting of 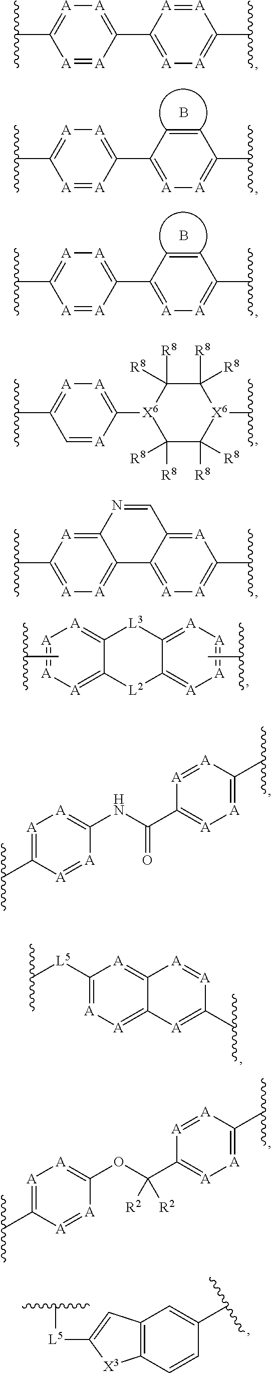 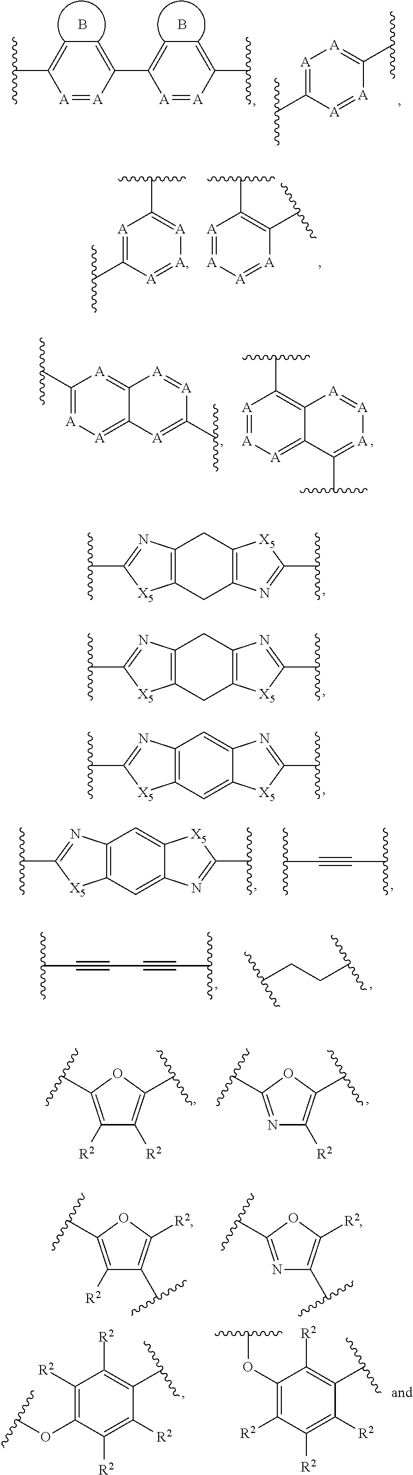 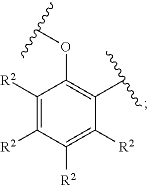
L² is selected from the group consisting of —C(═O)—, —(CH₂CH₂)—, —(CH₂O)—, —(CH₂S)—, —(CH═CH)—, —(CH═N)—, —NH—, O (oxygen), S (sulfur), and —CH₂—;
L³ is selected from the group consisting of 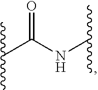
—(NR⁹)—, O (oxygen), S (sulfur), and —CH₂—;
L⁵ is selected from the group consisting of 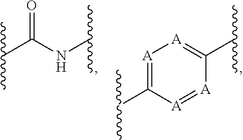
O (oxygen), NH, and —(CH═CH)—;
each X³ is separately selected from the group consisting of NH, O (oxygen), and S (sulfur);
each X⁵ is separately selected from the group consisting of —NH—, O (oxygen), S (sulfur), and —CH₂—;
each X⁶ is separately selected from the group consisting of N (nitrogen), and CR⁸;
each R⁸ is separately selected from the group consisting of hydrogen, C₁₋₆alkylOC₁₋₆alkyl, C₁₋₆alkylOC(═O)—, arylalkylOC(═O)—, halo, (R^{a}R^{b}N)alkyl, (R^{a}R^{b}N)C(═O)—, and C₁₋₆alkyl optionally substituted with up to 9 halo and up to 5 hydroxy;
each B is separately selected, wherein B is a fused optionally substituted saturated or unsaturated three- to seven-membered carbocyclic ring or a fused optionally substituted saturated or unsaturated three- to seven-membered heterocyclic ring, each optionally substituted with one or more R⁴ and;
each R⁴ is separately selected from the group consisting of C₁₋₆alkoxy, C₁₋₆alkylOC₁₋₆alkyl, C₁₋₆alkylOC(═O)—, arylalkylOC(═O)—, —COOH, halo, C₁₋₆haloalkyl, hydroxy, R^{a}R^{b}N—, (R^{a}R^{b}N)alkyl, (R^{a}R^{b}N)C(═O)—, and C₁₋₆alkyl optionally substituted with up to 9 halo and up to 5 hydroxy, or optionally two geminal R⁴ are together oxo.

81. A compound having the structure of Formula XV: 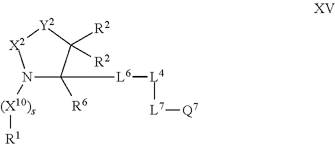
or a pharmaceutically acceptable salt thereof,
wherein:
Q⁷ is selected from the group consisting of 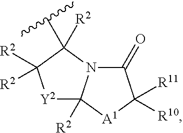
and 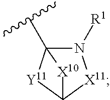
each X² is (C(R²)₂)_{q}, 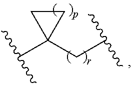
or X² is null;
each Y² is selected from O (oxygen), S (sulfur), S(O), SO₂, NR², and C(R²)₂ with the proviso that when X² is null Y² is C(R²)₂;
each X¹⁰ is (C(R²)₂)_{q};
each X¹¹ is separately selected from the group consisting of (C(R²)₂)_{q}, and 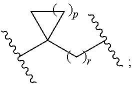
each Y¹¹ is separately selected from the group consisting of —O(C(R²)₂)ₙ—, —S(C(R²)₂)ₙ—, —S(O)(C(R²)₂)ₙ—, —SO₂(C(R²)₂)ₙ—, —NR²(C(R²)₂)ₙ—, and (C(R²)₂)_{q};
each R¹²R¹³N is separately selected, wherein R¹² and R¹³ are each separately selected from hydrogen, —[(Y¹⁴)(C(R²)₂)ᵣ(NR²)ₛ(C(R²)₂)ᵣ]—[Y¹⁴(C(R²)₂)ᵣ(NR²)ₛ(C(R²)₂)ᵣ]ₛ—(Y¹⁴)ₛ—R⁸⁰, —[(Y¹⁴)(C(R²)₂)ᵣ(NR²)ₛ(C(R²)₂)ᵣ]—Y¹⁴(C(R²)₂)ᵣO(C(R²)₂)ᵣ—(Y¹⁴)ₛ—R⁸⁰, alkoxyalkyl, alkoxyC(═O)—, C₂₋₆alkenyl, C₂₋₆alkynyl, C₃₋₇cycloalkyl, C₁₋₆alkylC(═O)—, C₃₋₇cycloalkylC(═O)—, C₁₋₆alkylsulfonyl, arylalkylOC(═O)—, aryl, arylalkyl, arylalkylC(═O)—, arylC(═O)—, arylsulfonyl, heterocyclyl, heteroaryl, heteroarylalkyl, heterocyclylalkyl, heterocyclylalkylC(═O)—, heterocyclylC(═O)—, heteroarylC(═O)—, heteroarylalkylC(═O)—, (R^{e}R^{f}N)alkyl, (R^{e}R^{f}N)alkylC(═O)—, and (R^{e}R^{f}N)C(═O)—,
said alkoxyalkyl, alkoxyC(═O)—, C₁₋₆ alkyl, C₃₋₇cycloalkyl, C₁₋₆ alkylC(═O)—, C₃₋₇cycloalkylC(═O)—, C₁₋₆alkylsulfonyl, arylalkylOC(═O)—, aryl, arylalkyl, arylalkylC(═O)—, arylC(═O)—, arylsulfonyl, heterocyclyl, heteroaryl, heteroarylalkyl, heterocyclylalkyl, heterocyclylalkylC(═O)—, heterocyclylC(═O)—, heteroarylC(═O)—, heteroarylalkylC(═O)—, and alkyl in (R^{e}R^{f}N)alkyl and (R^{e}R^{f}N)alkylC(═O)— are each optionally substituted with one or more R^{1ab};
or R¹²R¹³N is a heterocyclyl linked through a ring nitrogen atom optionally substituted with one or more of oxo, —[(Y¹⁴)(C(R²)₂)ᵣ(NR²)ₛ(C(R²)₂)ᵣ]—[Y¹⁴(C(R²)₂)ᵣ(NR²)ₛ(C(R²)₂)ᵣ]ₛ—(Y¹⁴)ₛ—R⁸⁰, —[(Y¹⁴)(C(R²)₂)ᵣ(NR²)ₛ(C(R²)₂)ᵣ]—_{Y}¹⁴(C(R²)₂)ᵣO(C(R²)₂)ᵣ—(Y¹⁴)ₛ—R⁸⁰, alkoxyalkyl, alkoxyC(═O)—, C₁₋₆alkyl, C₂₋₆alkenyl, C₂₋₆alkynyl, C₃₋₇cycloalkyl, C₁₋₆ alkylC(═O)—, C₃₋₇cycloalkylC(═O)—, C₁₋₆alkylsulfonyl, arylalkylOC(═O)—, aryl, arylalkyl, arylalkylC(═O)—, arylC(═O)—, arylsulfonyl, heterocyclyl, heteroaryl, heteroarylalkyl, heterocyclylalkyl, heterocyclylalkylC(═O)—, heterocyclylC(═O)—, heteroarylC(═O)—, heteroarylalkylC(═O)—, (R^{e}R^{f}N)alkyl, (R^{e}R^{f}N)alkylC(═O)—, and (R^{e}R^{f}N)C(═O)—,
said alkoxyalkyl, alkoxyC(═O)—, C₁₋₆ alkyl, C₃₋₇cycloalkyl, C₁₋₆alkylC(═O)—, C₃₋₇cycloalkylC(═O)—, C₁₋₆alkylsulfonyl, arylalkylOC(═O)—, aryl, arylalkyl, arylalkylC(═O)—, arylC(═O)—, arylsulfonyl, heterocyclyl, heteroaryl, heteroarylalkyl, heterocyclylalkyl, heterocyclylalkylC(═O)—, heterocyclylC(═O)—, heteroarylC(═O)—, heteroarylalkylC(═O)—, and alkyl in (R^{e}R^{f}N)alkyl and (R^{e}R^{f}N)alkylC(═O)— are each optionally substituted with one or more R^{1ab};
each R^{1ab} is separately selected from the group consisting of —[(Y¹⁴)(C(R²)₂)ᵣ(NR²)ₛ(C(R²)₂)ᵣ]-[Y¹⁴(C(R²)₂)ᵣ(NR²)ₛ(C(R²)₂)ᵣ]ₛ—(Y¹⁴)ₛ—R⁸⁰, —[(Y¹⁴)(C(R²)₂)ᵣ(NR²)ₛ(C(R²)₂)ᵣ]—Y¹⁴(C(R²)₂)ᵣO(C(R²)₂)ᵣ—(Y¹⁴)ₛ—R⁸⁰, —C(R^{2a})₂NR^{3a}R^{3b}, alkoxyalkyl, C₁₋₆alkylOC(═O)—, C₁₋₆alkylOC(═O)C₁₋₆alkyl, C₁₋₆alkylC(═O)C₁₋₆alkyl, aryl, aryl(CH₂)ₙ—, aryl(CH₂)ₙO—, aryl(CH═CH)ₘ—, arylalkylO—, arylalkyl, arylOalkyl, cycloalkyl, (cycloalkyl)(CH═CH)ₘ—, (cycloalkyl)alkyl, cycloalkylOalkyl, heterocyclyl, heterocyclyl(CH═CH)ₘ—, heterocyclylalkoxy, heterocyclylalkyl, heterocyclylOalkyl, hydroxyalkyl, R^{c}R^{d}N—, R^{c}R^{d}N(CH₂)ₙ—, (R^{c}R^{d}N)(CH═CH)ₘ—, (R^{c}R^{d}N)alkyl, (R^{c}R^{d}N)C(═O)—, C₁₋₆alkoxy optionally substituted with up to 9 halo, and C₁₋₆alkyl optionally substituted with up to 9 halo, said aryl and heteroaryl each optionally substituted with cyano, halo, nitro, hydroxyl, C₁₋₆alkoxy optionally substituted with up to 9 halo, and C₁₋₆alkyl optionally substituted with up to 9 halo;
each R⁸⁰ is separately selected from the group consisting of hydrogen, alkoxyalkyl, C₁₋₆alkyl, C₃₋₇cycloalkyl, aryl, arylalkyl, heterocyclyl, heterocyclylalkyl, heteroaryl, heteroarylalkyl, heterocyclylalkyl, and (R^{e}R^{f}N)alkyl, said alkoxyalkyl, C₁₋₆alkyl, C₃₋₇cycloalkyl, aryl, arylalkyl, heterocyclyl, heterocyclylalkyl, heteroaryl, heteroarylalkyl, heterocyclylalkyl, and alkyl in (R^{e}R^{f}N)alkyl are each optionally substituted with one or more R^{1ac};
each R^{1ac} is separately selected from the group consisting of —C(R^{2a})₂NR^{3a}R^{3b}, alkoxyalkyl, C₁₋₆alkylOC(═O)—, C₁₋₆alkylOC(═O)C₁₋₆alkyl, C₁₋₆alkylC(═O)C₁₋₆alkyl, aryl, aryl(CH₂)ₙ—, aryl(CH₂)ₙO—, aryl(CH═CH)ₘ—, arylalkylO—, arylalkyl, arylOalkyl, cycloalkyl, (cycloalkyl)(CH═CH)ₘ—, (cycloalkyl)alkyl, cycloalkylOalkyl, heterocyclyl, heterocyclyl(CH═CH)ₘ—, heterocyclylalkoxy, heterocyclylalkyl, heterocyclylOalkyl, hydroxyalkyl, R^{c}R^{d}N—, R^{c}R^{d}N(CH₂)ₙ—, (R^{c}R^{d}N)(CH═CH)ₘ—, (R^{c}R^{d}N)alkyl, (R^{c}R^{d}N)C(═O)—, C₁₋₆alkoxy optionally substituted with up to 9 halo, and C₁₋₆alkyl optionally substituted with up to 9 halo, said aryl and heteroaryl each optionally substituted with cyano, halo, nitro, hydroxyl, C₁₋₆alkoxy optionally substituted with up to 9 halo, and C₁₋₆alkyl optionally substituted with up to 9 halo;
each Y¹⁴ is separately selected from the group consisting of —C(═O)—, —S(═O)—, —C(═S)—, —S(═O)₂—, —C(═O)O—, —C(═O)NR^{2c}—, —S(═O)₂NR^{2c}—, —C(═O)NR^{2c}C(═O)—, and —C(CF₃)₂NR^{2c}—,
R¹ is selected from the group consisting of R^{1aa}, R^{1a}C(═O)— and R^{1a}C(═S)—;
each R^{1a} is separately selected from the group consisting of —C(R^{2a})₂NR^{3a}R^{3b}, alkoxyalkyl, C₁₋₆alkylOC(═O)—, C₁₋₆alkylOC(═O)C₁₋₆alkyl, C₁₋₆alkylC(═O)C₁₋₆alkyl, aryl, aryl(CH₂)ₙ—, aryl(CH₂)ₙO—, aryl(CH═CH)ₘ—, arylalkylO—, arylalkyl, arylOalkyl, cycloalkyl, (cycloalkyl)(CH═CH)ₘ—, (cycloalkyl)alkyl, cycloalkylOalkyl, heterocyclyl, heterocyclyl(CH═CH)ₘ—, heterocyclylalkoxy, heterocyclylalkyl, heterocyclylOalkyl, hydroxyalkyl, R^{c}R^{d}N—, R^{c}R^{d}N(CH₂)ₙ—, (R^{c}R^{d}N)(CH═CH)ₘ—, (R^{c}R^{d}N)alkyl, (R^{c}R^{d}N)C(═O)—, C₁₋₆alkoxy optionally substituted with up to 9 halo, and C₁₋₆alkyl optionally substituted with up to 9 halo, said aryl and heteroaryl each optionally substituted with cyano, halo, nitro, hydroxyl, C₁₋₆alkoxy optionally substituted with up to 9 halo, and C₁₋₆alkyl optionally substituted with up to 9 halo;
R^{1aa} is selected from the group consisting of —C(R^{2a})₂NR^{3a}R^{3b}, —[(Y¹⁴)(C(R²)₂)ᵣ(NR²)ₛ(C(R²)₂)ᵣ]—[Y¹⁴(C(R²)₂)ᵣ(NR²)ₛ(C(R²)₂)ᵣ]ₛ—(Y¹⁴)ₛ—R⁸⁰, —[(Y¹⁴)(C(R²)₂)ᵣ(NR²)ₛ(C(R²)₂)ᵣ]—Y¹⁴(C(R²)₂)ᵣO(C(R²)₂)ᵣ—(Y¹⁴)ₛ—R⁸⁰, alkoxyalkyl, alkoxyC(═O)—, C₁₋₆alkyl, C₂₋₆alkenyl, C₂₋₆alkynyl, C₃₋₇cycloalkyl, C₁₋₆alkylC(═O)—, C₃₋₇cycloalkylC(═O)—, C₁₋₆alkylsulfonyl, arylalkylOC(═O)—, aryl, arylalkyl, arylalkylC(═O)—, arylC(═O)—, arylsulfonyl, heterocyclyl, heteroaryl, heteroarylalkyl, heterocyclylalkyl, heterocyclylalkylC(═O)—, heterocyclylC(═O)—, heteroarylC(═O)—, heteroarylalkylC(═O)—, (R^{e}R^{f}N)alkyl, (R^{e}R^{f}N)alkylC(═O)—, and (R^{e}R^{f}N)C(═O)—,
said alkoxyalkyl, alkoxyC(═O)—, C₁₋₆alkyl, C₃₋₇cycloalkyl, C₁₋₆alkylC(═O)—, C₃₋₇cycloalkylC(═O)—, C₁₋₆alkylsulfonyl, arylalkylOC(═O)—, aryl, arylalkyl, arylalkylC(═O)—, arylC(═O)—, arylsulfonyl, heterocyclyl, heteroaryl, heteroarylalkyl, heterocyclylalkyl, heterocyclylalkylC(═O)—, heterocyclylC(═O)—, heteroarylC(═O)—, heteroarylalkylC(═O)—, and alkyl in (R^{e}R^{f}N)alkyl and (R^{e}R^{f}N)alkylC(═O)— are each optionally substituted with one or more R^{1ab};
each R¹⁰ is R^{c}R^{d}N—;
each R¹¹ is separately selected from the group consisting of H (hydrogen), alkoxyalkyl, C₁₋₆alkylOC(═O)C₁₋₆alkyl, C₁₋₆alkylC(═O)C₁₋₆alkyl, aryl(CH₂)ₙ—, arylalkyl, arylOalkyl, cycloalkyl, (cycloalkyl)alkyl, cycloalkylOalkyl, heterocyclylalkyl, heterocyclylOalkyl, hydroxyalkyl, R^{c}R^{d}N(CH₂)ₙ—, (R^{c}R^{d}N)alkyl, and C₁₋₆alkyl optionally substituted with up to 9 halo;
each R^{c}R^{d}N is separately selected, wherein R^{c} and R^{d} are each separately selected from hydrogen, alkoxyC(═O)—, C₁₋₆alkyl, C₁₋₆alkylC(═O)—, C₁₋₆alkylsulfonyl, arylalkylOC(═O)—, arylalkyl, arylalkylC(═O)—, arylC(═O)—, arylsulfonyl, heterocyclylalkyl, heterocyclylalkylC(═O)—, heterocyclylC(═O)—, (R^{e}R^{f}N)alkyl, (R^{e}R^{f}N)alkylC(═O)—, and (R^{e}R^{f}N)C(═O)—, wherein the alkyl part of arylalkyl, arylalkylC(═O)—, heterocyclylalkyl, and heterocyclylalkylC(═O)— are each optionally substituted with one R^{e}R^{f}N— group; and wherein the aryl part of arylalkyl, arylalkylC(═O)—, arylC(═O)—, and arylsulfonyl, and the heterocyclyl part of heterocyclylalkyl, heterocyclylalkylC(═O)—, and heterocyclylC(═O)— are each optionally substituted with up to three substituents each independently selected from the group consisting of cyano, halo, nitro, C₁₋₆alkoxy optionally substituted with up to 9 halo, and C₁₋₆alkyl optionally substituted with up to 9 halo;
each R^{e}R^{f}N is separately selected, wherein R^{e} and R^{f} are each separately selected from hydrogen, C₁₋₆alkyl, aryl, arylalkyl, cycloalkyl, (cyclolalkyl)alkyl, heterocyclyl, heterocyclylalkyl, (R^{x}R^{y}N)alkyl, and (R^{x}R^{y}N)C(═O)—;
each R^{x}R^{y}N is separately selected, wherein R^{x} and R^{y} are each separately selected from hydrogen, C₁₋₆alkylOC(═O)—, C₁₋₆alkyl, C₁₋₆alkylC(═O)—, aryl, arylalkyl, cycloalkyl, and heterocyclyl;
each C(R^{2a})₂ is separately selected, wherein each R^{2a} is separately selected from the group consisting of hydrogen, C₁₋₆alkyl optionally substituted with up to 9 halo, aryl(CH₂)ₙ—, and heteroaryl(CH₂)ₙ—, said aryl and heteroaryl each optionally substituted with cyano, halo, nitro, hydroxyl, C₁₋₆alkoxy optionally substituted with up to 9 halo, and C₁₋₆alkyl optionally substituted with up to 9 halo, or optionally C(R^{2a})₂ is 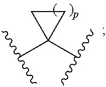
each R^{3a} is separately selected from the group consisting of hydrogen, and optionally substituted C₁₋₆alkyl;
each R^{3b} is separately selected from the group consisting of optionally substituted C₁₋₆alkyl, heteroaryl, —(CH₂)ₙC(═O)NR^{4a}R^{4b}, —(CH₂)ₙC(═O)OR^{5a}, and —(CH₂)ₙC(═O)R^{6a} said heteroaryl optionally substituted with cyano, halo, nitro, hydroxyl, C₁₋₆alkoxy optionally substituted with up to 9 halo, and C₁₋₆alkyl optionally substituted with up to 9 halo;
each R^{4a}R^{4b}N is separately selected, wherein R^{4a} and R^{4b} are each separately selected from the group consisting of hydrogen, optionally substituted C₁₋₆alkyl, and aryl(CH₂)ₙ—;
each R^{5a} is separately selected from the group consisting of optionally substituted C₁₋₆alkyl, and aryl(CH₂)ₙ—;
each R^{6a} is separately selected from the group consisting of optionally substituted C₁₋₆alkyl, and aryl(CH₂)ₙ—;
each A¹ is separately selected from the group consisting of C₂₋₆alkenyl, C₁₋₆alkyl, and —(CH₂)ₙ—O—(CH₂)ₘ—, each optionally substituted with one or more R²;
each R² is separately selected, wherein R² is selected from the group consisting of hydrogen, halo, hydroxy, C₁₋₆alkoxy, C₁₋₆alkyl, C₂₋₆alkenyl, C₂₋₆alkynyl, alkyoxyalkyl, C₃₋₇cycloalkyl, aryl, arylalkyl, heteroaryl, heteroarylalkyl, heterocyclyl, heterocyclylalkyl, (R^{e}R^{f}N)alkyl, R^{a}R^{b}N—, said C₁₋₆alkyl optionally substituted with one or more halo, —OR^{2b}, —C(═O)OR^{2b}, —C(═O)NHR^{2b}, —NHC(═NH)NHR^{2b}, —NHR^{2b}, SR^{2b}, imidazolyl, indolyl, —SCH₃, phenyl, and 4-hydroxyphenyl, said C₁₋₆alkoxy, alkoxyalkyl, aryl, C₂₋₆alkenyl, C₂₋₆alkynyl, C₃₋₇cycloalkyl, arylalkyl, heterocyclyl, heteroaryl, heteroarylalkyl, heterocyclylalkyl, and alkyl in (R^{e}R^{f}N)alkyl each optionally substituted with one or more R⁴, or optionally two vicinal R² and the carbons to which they are attached are together a fused three- to six-membered carbocyclic ring optionally substituted with up to two C₁₋₆alkyl groups;
R^{2b} is selected from the group consisting of hydrogen, C₁₋₆alkyl, C₂₋₆alkenyl, C₂₋₆alkynyl, C₃₋₇cycloalkyl, aryl, arylalkyl, heteroaryl, heteroarylalkyl, heterocyclyl, and heterocyclylalkyl;
each R^{2c} is selected from the group consisting of hydrogen, C₁₋₆alkyl, C₂₋₆alkenyl, C₂₋₆alkynyl, C₃₋₇cycloalkyl, aryl, arylalkyl, heteroaryl, heteroarylalkyl, heterocyclyl, and heterocyclylalkyl, said alkyl optionally substituted with R^{e}R^{f}N—, alkoxy, or C₁₋₆alkylS—;
each R^{a}R^{b}N is separately selected, wherein R^{a} and R^{b} are each separately selected from the group consisting of hydrogen, C₂₋₆alkenyl, and C₁₋₆alkyl;
L⁴ is selected from the group consisting of -(J²)ₛ-(L⁵)ₛ-(J²)ₛ-(L⁵)ₛ-J²-, 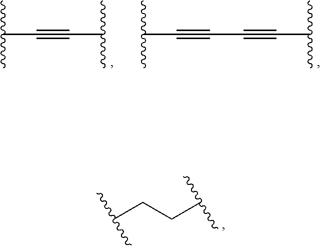
—C(═O)—, O (oxygen), —OC(R²)₂—, 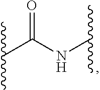
—C(CF₃)₂NR^{2c}—, NH, and —(CH═CH)—;
each J² is separately selected from the group consisting of aryl, heteroaryl, heterocyclyl, cycloalkyl, cycloalkenyl, and polycyclic hydrocarbon, each optionally substituted with one or more R¹⁵;
each R¹⁴ is separately selected from the group consisting of hydroxy, C₁₋₆alkoxy optionally substituted with up to 9 fluoro, C₁₋₆alkylOC₁₋₆alkyl, C₁₋₆alkylOC(═O)—, arylalkylOC(═O)—, —COOH, halo, and C₁₋₆alkyl optionally substituted with up to 9 halo;
each R¹⁵ is separately selected from the group consisting of halo, hydroxy, C₁₋₆alkylOC₁₋₆alkyl, C₁₋₆alkylOC(═O)—, arylalkylOC(═O)—, —COOH, R⁹, R^{x}R^{y}N, R^{x}R^{y}NC(═O), R^{x}R^{y}NC₁₋₆alkyl, heteroaryl, aryl, C₁₋₆alkyl optionally substituted with up to 9 halo, C₁₋₆alkoxy optionally substituted with up to 9 halo, C₁₋₆haloalkyl, R^{a}R^{b}N—, (R^{a}R^{b}N)alkyl, (R^{a}R^{b}N)C(═O)—, and C₁₋₆alkyl substituted with up to 5 hydroxy, said substituent aryl and heteroaryl are each optionally substituted with one or more R¹⁴, or optionally two vicinal R¹⁵ and the carbons to which they are attached are together a fused three- to six-membered carbocyclic ring optionally substituted with up to two C₁₋₆alkyl groups, or optionally two geminal R¹⁵ and the carbon to which they are attached are together a three- to six-membered carbocyclic ring optionally substituted with up to two C₁₋₆alkyl groups, or optionally two geminal R¹⁵ are together oxo;
each L⁵ is separately selected from the group consisting of 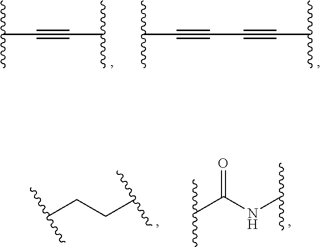
—C(CF₃)₂NR^{2c}—, 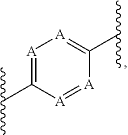
—C(R²)₂—, —C(R²)₂O—, —C(═O)—, O (oxygen), NH, and —(CH═CH)—;
each A is separately selected from the group consisting of CR³ and N (nitrogen);
L⁶ is selected from the group consisting of 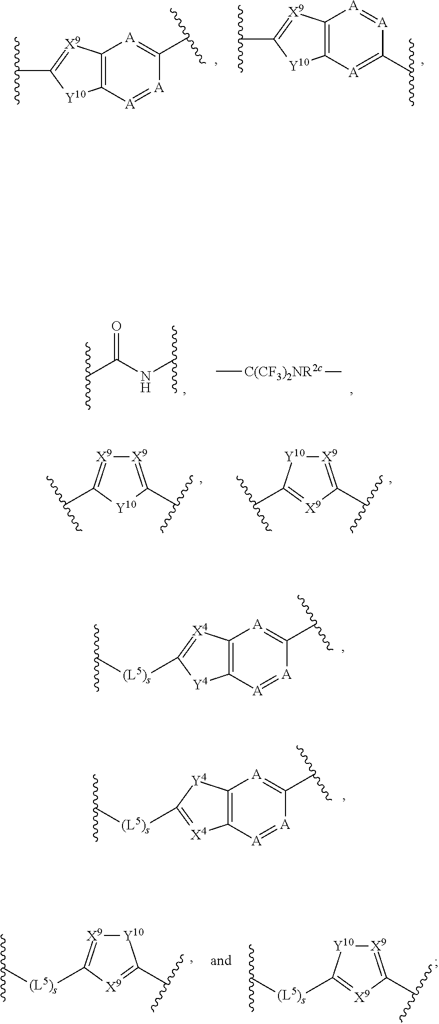
L⁷ is selected from the group consisting of 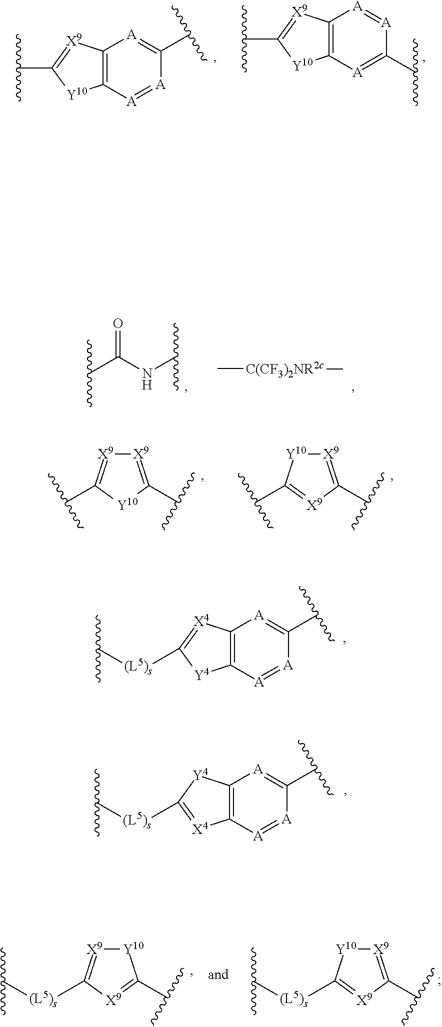
each X⁴ is separately selected from the group consisting of CR⁴ and N (nitrogen);
each Y⁴ is separately selected from the group consisting of C(R⁴)₂, NR⁴, O (oxygen), and S (sulfur);
each X⁹ is separately selected from the group consisting of CH and N (nitrogen);
each Y¹⁰ is separately selected from the group consisting of —CH₂— and —NH—;
each m separately is 1 or 2;
each n separately is 0, 1 or 2;
each p separately is 1, 2, 3 or 4;
each q separately is 1, 2, 3, 4 or 5;
each r separately is 0, 1, 2, 3, or 4;
each s separately is 0 or 1;
each R³ is separately selected from the group consisting of hydrogen, C₁₋₆alkoxy, C₁₋₆alkylOC₁₋₆alkyl, C₁₋₆alkylOC(═O)—, arylalkylOC(═O)—, —COOH, halo, hydroxy, R^{a}R^{b}N—, (R^{a}R^{b}N)alkyl, (R^{a}R^{b}N)C(═O)—, and C₁₋₆alkyl optionally substituted with up to 9 halo and up to 5 hydroxy;
each R⁴ is separately selected from the group consisting of H (hydrogen), C₁₋₆alkoxy, C₁₋₆alkylOC₁₋₆alkyl, C₁₋₆alkylOC(═O)—, arylalkylOC(═O)—, —COOH, halo, C₁₋₆haloalkyl, hydroxy, R^{a}R^{b}N—, (R^{a}R^{b}N)alkyl, (R^{a}R^{b}N)C(═O)—, and C₁₋₆alkyl optionally substituted with up to 9 halo and up to 5 hydroxy, or optionally two geminal R⁴ are together oxo;
R⁶ is selected from the group consisting of hydrogen, halo, hydroxy, C₁₋₆alkoxy, C₁₋₆alkyl, C₂₋₆alkenyl, C₂₋₆alkynyl, alkyoxyalkyl, C₃₋₇cycloalkyl, aryl, arylalkyl, heteroaryl, heteroarylalkyl, heterocyclyl, heterocyclylalkyl, (R^{e}R^{f}N)alkyl, R^{a}R^{b}N—, said C₁₋₆alkyl optionally substituted with one or more halo, —OR^{2b}, —C(═O)OR^{2b}, —C(═O)NHR^{2b}, —NHC(═NH)NHR^{2b}, —NHR^{2b}, SR^{2b}, imidazolyl, indolyl, —SCH₃, phenyl, and 4-hydroxyphenyl, and said C₁₋₆alkoxy, alkoxyalkyl, aryl, C₂₋₆alkenyl, C₂₋₆alkynyl, C₃₋₇cycloalkyl, arylalkyl, heterocyclyl, heteroaryl, heteroarylalkyl, heterocyclylalkyl, and alkyl in (R^{e}R^{f}N)alkyl are each optionally substituted with one or more R⁴;
R⁹ is selected from the group consisting of hydrogen and —C(═O)R^{9a};
R^{9a} is selected from the group consisting of —NR^{9b}R^{9c}, —OR^{9d}, C₁₋₆alkyl optionally substituted with up to 9 halo, and optionally substituted aryl;
R^{9b} is selected from the group consisting of hydrogen, C₁₋₆alkyl optionally substituted with up to 9 halo, and optionally substituted aryl;
R^{9c} is selected from the group consisting of C₁₋₆alkyl optionally substituted with up to 9 halo, and optionally substituted aryl; and
R^{9d} is selected from the group consisting of C₁₋₆alkyl optionally substituted with up to 9 halo, and optionally substituted aryl,
provided that the compound is not selected from the group consisting of:

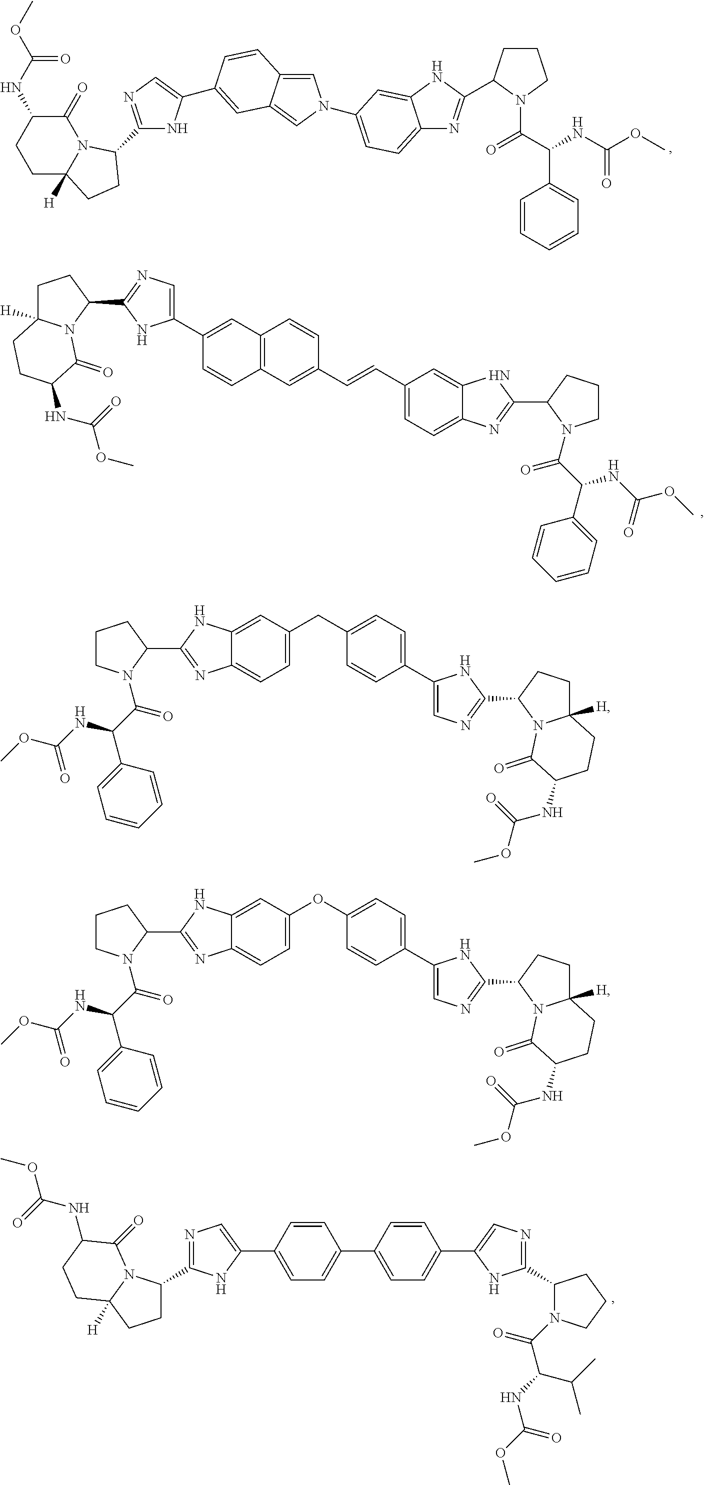

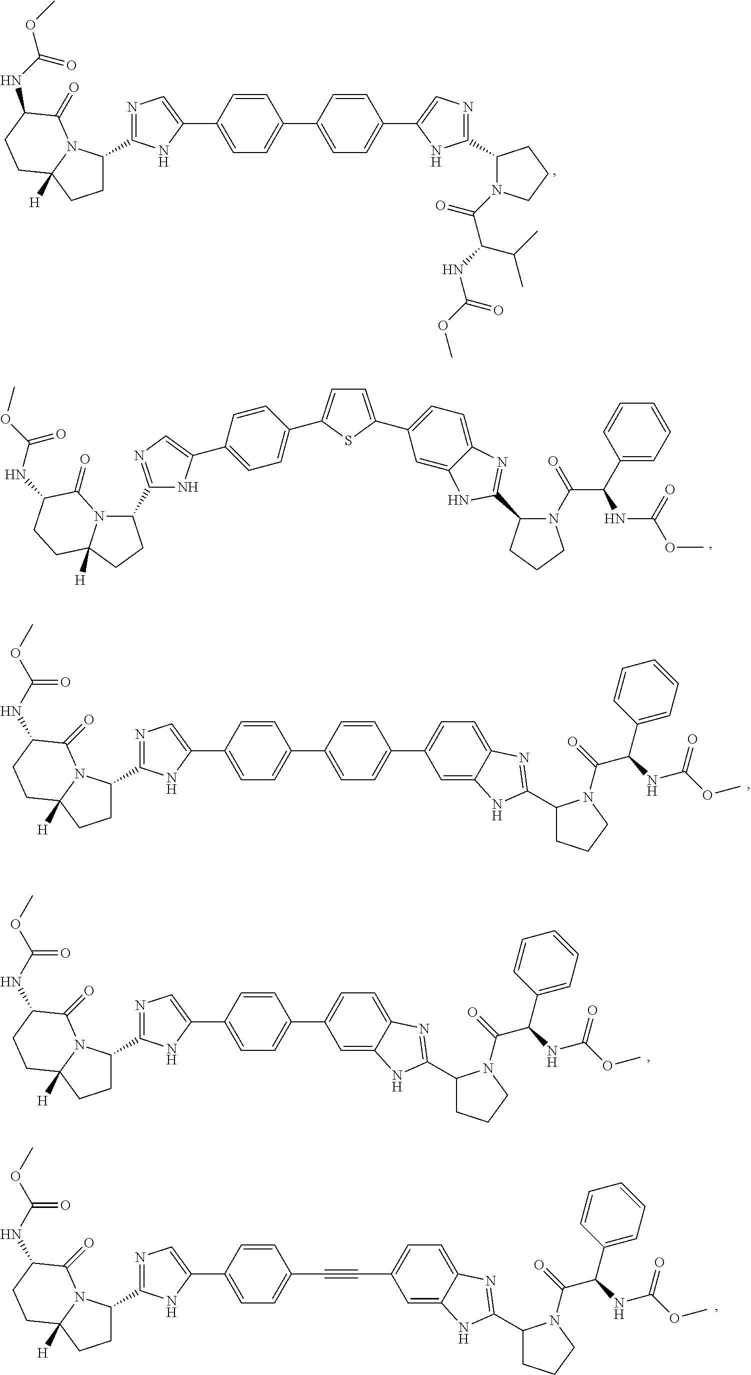

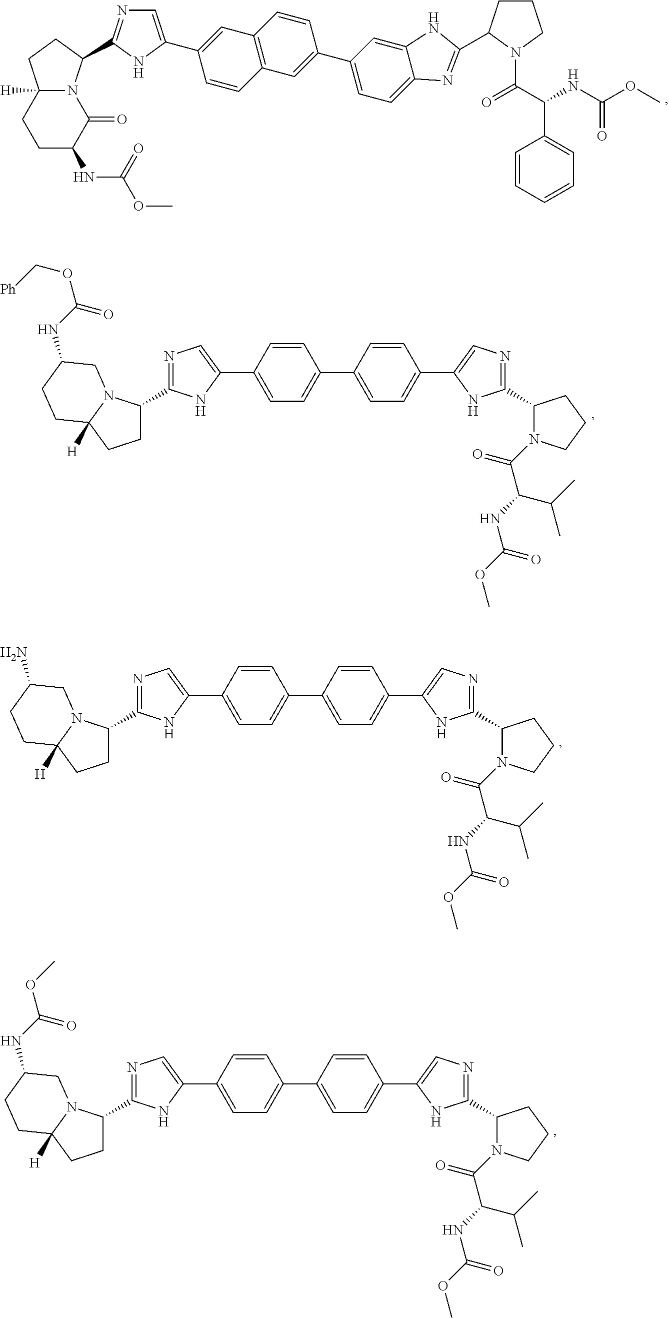

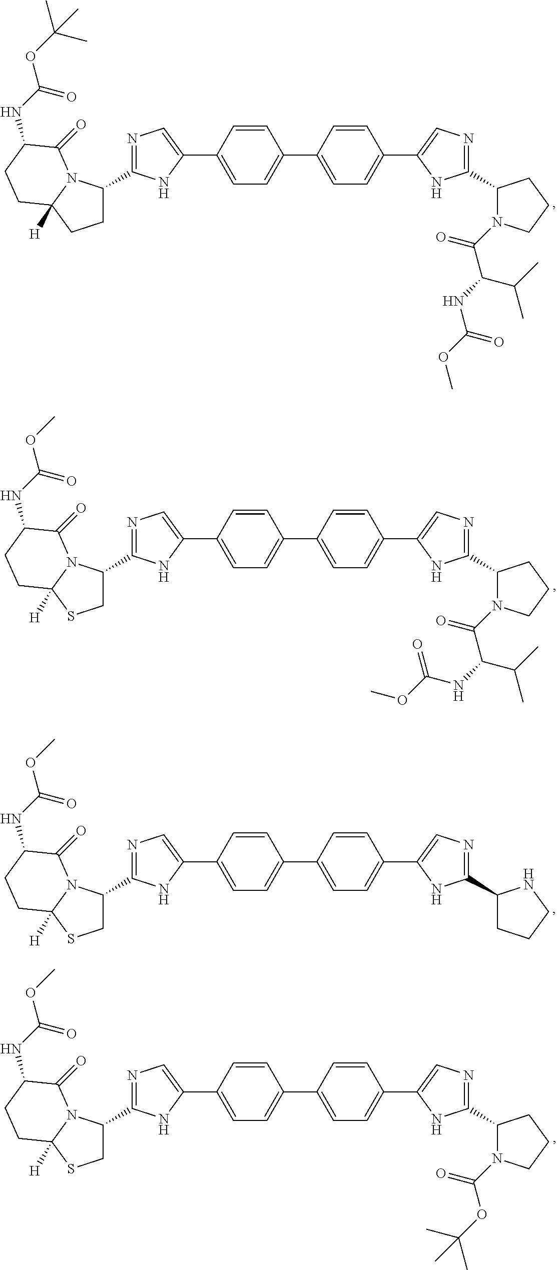

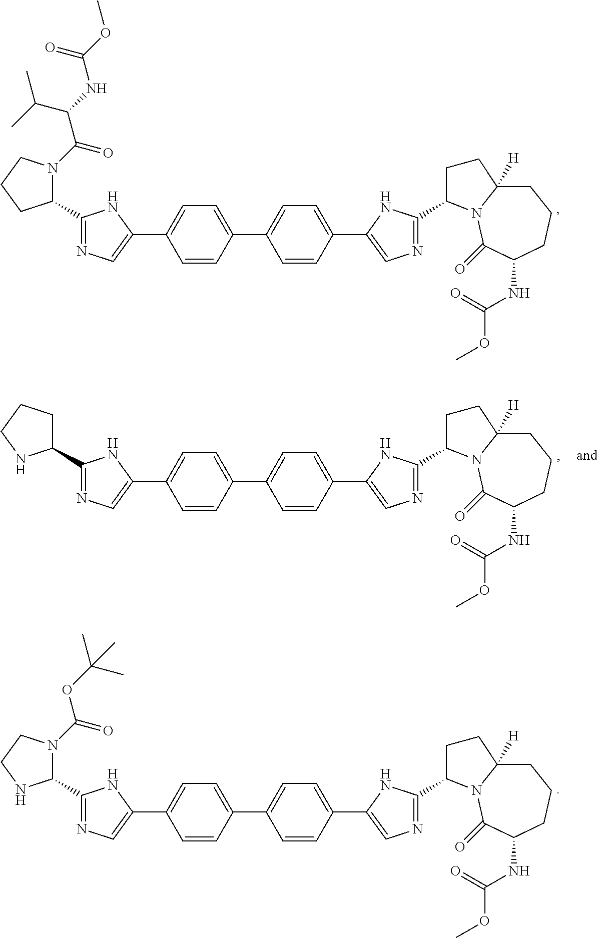

82. The compound of claim 81, wherein
L⁴ is selected from the group consisting of 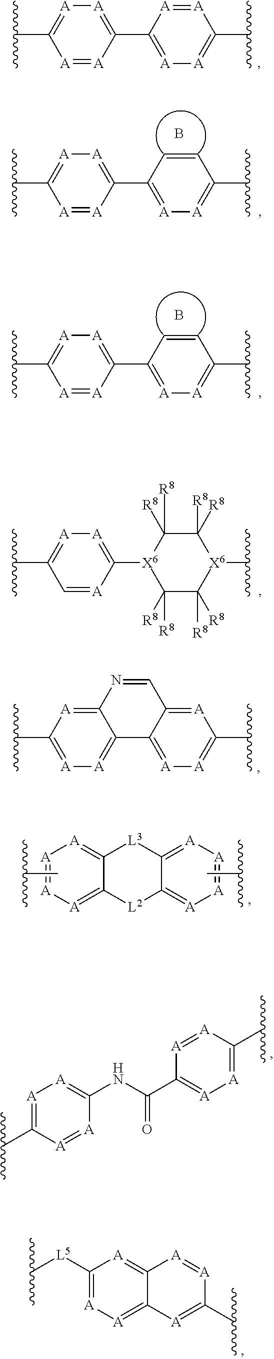 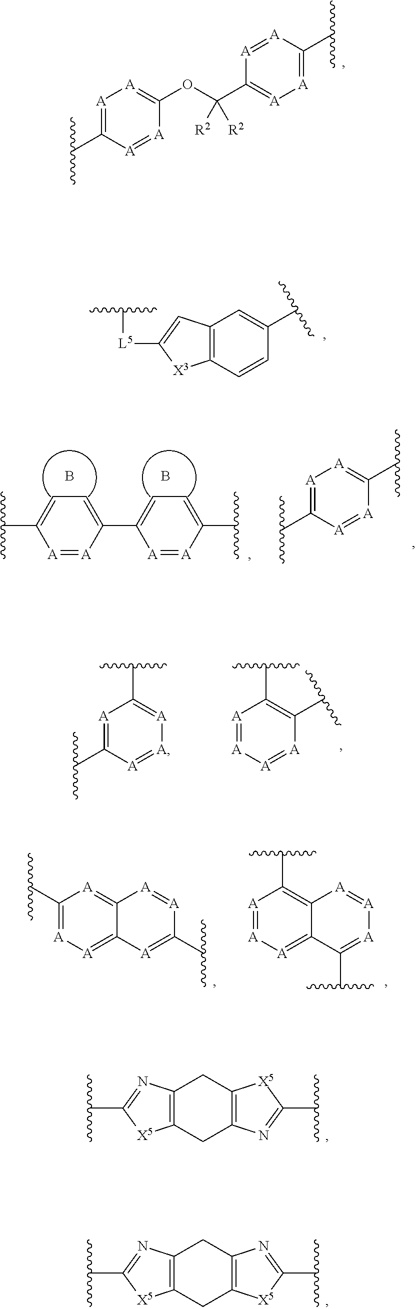 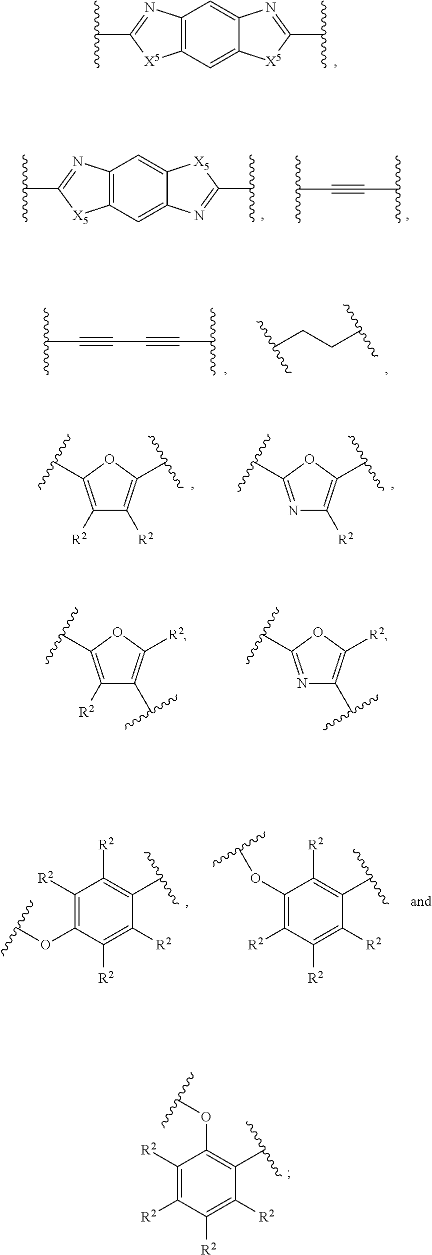
L² is selected from the group consisting of —C(═O)—, —(CH₂CH₂)—, —(CH₂O)—, —(CH₂S)—, —(CH═CH)—, —(CH═N)—, —NH—, O (oxygen), S (sulfur), and —CH₂—;
L³ is selected from the group consisting of 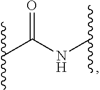
—(NR⁹)—, O (oxygen), S (sulfur), and —CH₂—;
L⁵ is selected from the group consisting of 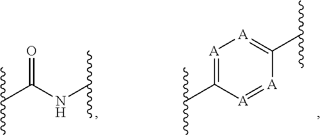
O (oxygen), NH, and —(CH═CH)—;
each X³ is separately selected from the group consisting of NH, O (oxygen), and S (sulfur);
each X⁵ is separately selected from the group consisting of —NH—, O (oxygen), S (sulfur), and —CH₂—;
each X⁶ is separately selected from the group consisting of N (nitrogen), and CR⁸; and
each B is separately selected, wherein B is a fused optionally substituted saturated or unsaturated three- to seven-membered carbocyclic ring or a fused optionally substituted saturated or unsaturated three- to seven-membered heterocyclic ring, each optionally substituted with one or more R⁴.

83. The compound of claim 81, wherein R⁶ is C₁₋₆alkyl optionally substituted with up to 9 halo.

84. The compound of claim 81, wherein L⁶ is selected from the group consisting of 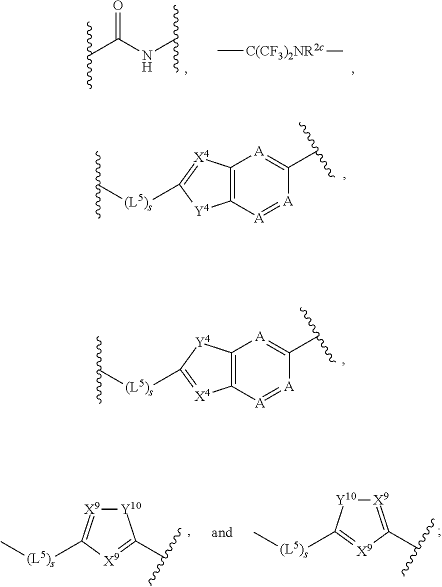
and
L⁷ is selected from the group consisting of

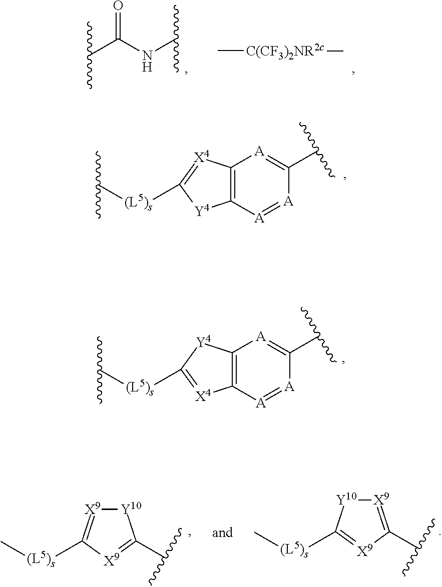

85. The compound of claim 81, having the structure: 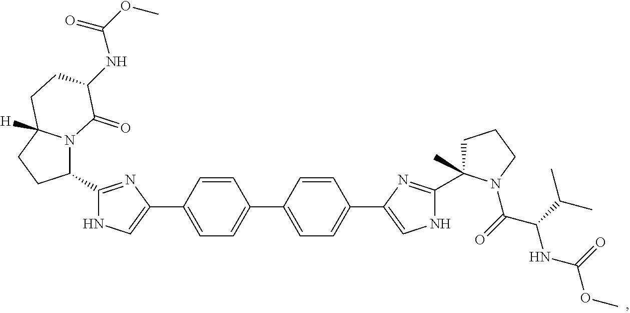
or a pharmaceutically acceptable salt thereof.

86. A pharmaceutical composition comprising a pharmaceutically acceptable excipient and a compound of claim 1.

87. A method of treating HCV infection in an individual, the method comprising administering to the individual an effective amount of a compound of claim 1.

88. The method of claim 87, further comprising identifying a subject suffering from a hepatitis C infection.

89. A method of treating liver fibrosis in an individual, the method comprising administering to the individual an effective amount of a compound of claim 1.

90. The method of claim 89, further comprising identifying a subject suffering from a hepatitis C infection.

91. A method of increasing liver function in an individual having a hepatitis C virus infection, the method comprising administering to the individual an effective amount of a compound of claim 1.

92. The method of claim 91, further comprising identifying a subject suffering from a hepatitis C infection.
